(19) United States
(12) Patent Application Publication
(10) Pub. No.: US 2015/0031668 A1
(43) Pub. Date: Jan. 29, 2015

(54) CHROMAN DERIVATIVES AS TRPM8 INHIBITORS

(71) Applicant: AMGEN INC., Thousand Oaks, CA (US)

(72) Inventors: Kaustav Biswas, Agoura Hills, CA (US); Jian J. Chen, Camarillo, CA (US); Vijay Keshav Gore, Aliso Viejo, CA (US); Scott Harried, Pittsburgh, PA (US); Daniel B. Horne, Simi Valley, CA (US); Matthew R. Kaller, Ventura, CA (US); Vu Van Ma, Oak Park, CA (US); Kelvin Sham, Thousand Oaks, CA (US); James Brown, Moorpark, CA (US); Wenge Zhong, Thousand Oaks, CA (US); Thomas T. Nguyen, Newbury Park, CA (US)

(21) Appl. No.: 14/331,426

(22) Filed: Jul. 15, 2014

Related U.S. Application Data
(60) Provisional application No. 61/847,867, filed on Jul. 18, 2013.

(51) Int. Cl. C07D 491/052; C07D 498/04; C07D 471/04

(52) U.S. Cl. CPC C07D 491/052; C07D 471/04; C07D 498/04
USPC 514/210.18; 546/113; 514/215; 540/580; 514/300; 546/122; 546/115; 514/302; 514/278; 546/15; 514/249; 544/355; 544/236; 514/248; 514/230.5; 544/91

(57) ABSTRACT

Chroman compounds and derivatives of Formula I are useful inhibitors of TRPM8. Such compounds are useful in treating a number of TRPM8 mediated disorders and conditions and may be used to prepare medicaments and pharmaceutical compositions useful for treating such disorders and conditions. Examples of such disorders include, but are not limited to, migraines and neuropathic pain. Compounds of Formula I have the following structure:

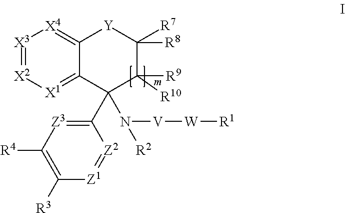

where the definitions of the variables are provided herein.

### CROSS REFERENCES TO RELATED APPLICATIONS

This application claims the benefit of U.S. Provisional Application No. 61/847,867, filed on Jul. 18, 2013, which is hereby incorporated by reference in its entirety and for all purposes as if fully set forth herein.

### FIELD OF THE INVENTION

The present invention relates to chroman compounds and related derivatives that have TRPM8 antagonist properties and are useful in preparing medicaments and compositions and in treating diseases and conditions such as those mediated by TRPM8. The compounds and compositions may be used to treat various diseases or conditions modulated by TRPM8 such as, but not limited to, migraines and neuropathic pain.

### BACKGROUND OF THE INVENTION

Cold sensation is derived from activation of the somatosensory system by a cold stimulus. Calcium imaging and patch clamp experiments in dissociated trigeminal and dorsal root ganglia neurons have revealed cold stimuli induced calcium influx, suggesting the direct opening of a calcium-permeable ion channels by cold (Thut et al., 2003; Reid, 2005). A recently cloned non-selective cation channel, TRPM8 (transient receptor potential melastatin 8) or trp-p8 (identified as a prostate-specific gene, up-regulated in prostate cancer and other malignancies, (Tsavaler et al., 2001)) is activated by cold stimulus of 10 to 24° C. temperature (McKemy et al., 2002; Peier et al., 2002). In addition, TRPM8 is also activated by compounds that elicit cool sensation such as menthol, icilin (AG-3-5) (McKemy et al., 2002), and the endogenous lipid PIP₂ (Rohacs et al., 2005). Correlating with the cold sensitivity of both A delta and C-fibers, TRPM8 is highly expressed in sensory neurons of the trigeminal and dorsal root ganglia (McKemy et al., 2002; Peier et al., 2002; Thut et al., 2003). TRPM8 is also expressed in nerve fibers innervating urinary bladder in guinea pigs (Tsukimi et al., 2005) and humans (Mukerji et al., 2006) and believed to contribute to the bladder hypersensitivity.

Activation mechanism of TRPM8 by menthol and icilin appears to differ. Icilin requires calcium for robust activation of TRPM8, whereas menthol and cold do not (Chuang et al., 2004). Typically, activation by all these agonists follows a period of calcium-dependent desensitization. The domain swap analysis of chicken and rat TRPM8 and further mutational studies revealed that determinants of icilin sensitivity map to a region of TRPM8 that corresponds to the capsaicin binding site in TRPV1 transmembrane domain 3 to 4 region (Chuang et al., 2004).

Cold allodynia and mechanical hyperalgesia are associated with neuropathic pain in humans and in rodent models of neuropathic and chemotherapy-induced pain. TRPM8 is shown to mediate the analgesia by agonists such as menthol and icilin (by desensitization of the receptor) during experimental neuropathic pain in rodents (Proudfoot et al., 2006). Further, attenuation of cold sensation and cold allodynia after chronic constriction injury model of neuropathic pain in TRPM8 knockout mice (Colburn et al., 2007; Dhaka et al., 2007) suggests that antagonists of TRPM8 may be considered as pain therapeutics for chemotherapy-induced pain, neuropathic pain and bladder disorders.

Mint oil that contains menthol, an agonist of TRPM8 has been reported to alleviate pain in post-herpetic neuralgia (Davies et al., 2002), a neuropathic pain condition. Furthermore, oral or intracerebroventricular injection of menthol decreased nociceptive responses to hot-plate test and acetic acid-induced writhing in mice (Galeotti et al., 2002). These responses are believed to be mediated by the activation and desensitization of the TRPM8. These observations and the knockout mice studies indicate that TRPM8 modulation by antagonists might be beneficial for patients experiencing neuropathic pain.

A need exists for TRPM8 antagonist compounds that can be used to treat diseases and conditions mediated by TRPM8 such as, but not limited to, migraines and neuropathic pain and those other conditions described herein.

### SUMMARY OF THE INVENTION

The present invention comprises a new class of compounds useful in the treatment of diseases, such as TRPM8-mediated diseases and other maladies, such as inflammatory or neuropathic pain and diseases involving sensory nerve function such as asthma, rheumatoid arthritis, osteoarthritis, inflammatory bowel disorders, urinary incontinence, migraine and psoriasis. In particular, the compounds of the invention are useful for the treatment of acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, anxiety, depression, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders. Accordingly, the invention also comprises pharmaceutical compositions comprising the compounds, methods for the treatment of TRPM8-receptor-mediated diseases, such as inflammatory or neuropathic pain, asthma, rheumatoid arthritis, osteoarthritis, inflammatory bowel disorders, urinary incontinence, migraine and psoriasis diseases, using the compounds and compositions of the invention, and intermediates and processes useful for the preparation of the compounds of the invention.

In one aspect, the invention provides compounds of Formula I or a pharmaceutically-acceptable salt thereof, a tautomer thereof, a pharmaceutically-acceptable salt of the tautomer, a stereoisomer thereof, or a mixture thereof. Compounds of Formula I have the following structure

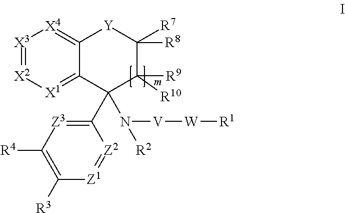

where

V is selected from —C(═O)— or —S(═O)₂—;

W is absent or is selected from —NH—, —NR^{1a}—, or O;

X¹ is selected from —CR⁵— or —N—;

X² is selected from —CR⁵— or —N—;

X³ is selected from —CR⁵— or —N—;

X⁴ is selected from —CR⁵— or —N—;

Y is selected from —O—, —CH₂—, —NH—, —NR^{1b}—, —CF₂—, —C(═O)—, —C(H)(F)—, or —C(H)(OH)—;

Z¹ is selected from —CR⁶— or —N—;

Z² is selected from —CR⁶— or —N—;

Z³ is selected from —CR⁶— or —N—;

wherein 0, 1, or 2 of X¹, X², X³, and X⁴ are N;

wherein 0, 1, or 2 of Z¹, Z², and Z³ are N;

m is 0, 1, or 2;

R¹ is C₁₋₆alk or a direct-bonded, C₁₋₂alk-linked, C₁₋₂alkO-linked, saturated, partially-saturated or unsaturated 3-, 4-, 5-, 6- or 7-membered monocyclic or 7-, 8-, 9-, 10- or 11-membered bicyclic ring containing 0, 1, 2, 3 or 4 heteroatoms selected from N, O and S, but containing no more than one O or S atom, the C₁₋₆alk and ring being substituted by 0, 1, 2 or 3 substituents independently selected from halo, oxo, C₁₋₆alk, C₁₋₆alkOH, C₁₋₆alk-C(═O)R^{a}, C₁₋₆alk-C(═O)OR^{a}, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, ═S, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the ring is additionally substituted by 0 or 1 directly bonded, SO₂ linked, C(═O) linked or CH₂ linked saturated, partially-saturated or unsaturated 3-, 4-, 5-, 6- or 7-membered monocyclic ring containing 0, 1, 2, 3 or 4 heteroatoms selected from N, O and S, but containing no more than one O or S atom, and substituted by 0, 1, 2 or 3 groups selected from halo, oxo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —SR^{a}, —S(═O)R^{a}, —S(═O)₂R^{a}, —S(═O)₂NR^{a}R^{a}, —NR^{a}R^{a}, and —N(R^{a})C(═O)R^{a};

R^{1a} is C₁₋₆alk;

R^{1b} is C₁₋₆alk or —C(═O)R^{b};

R² is H or C₁₋₆alk;

R³ is H, C₁₋₈alk, C₁₋₈alkOH, C₁₋₄haloalk, halo, cyano, R^{b}, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} or —NR^{a}C₂₋₆alkOR^{a};

R⁴ is H, C₁₋₆alk, —C₁₋₃haloalk, —OC₁₋₆alk, —OC₁₋₃haloalk, —N(C₁₋₆alk)C₁₋₆alk, —NHC₁₋₆alk, —NC(═O)C₁₋₆alk, —N(C₁₋₆alk)C₁₋₆alk, F, Cl, Br, CN, OH or NH₂; or R³ and R⁴ together form a four-atom unsaturated bridge containing 0 or 1 N atoms, wherein the bridge is substituted by 0, 1 or 2 R⁵ substituents;

R⁵ is, at each instance, independently selected from H, C₁₋₈alk, C₁₋₈alkOH, C₁₋₄haloalk, halo, cyano, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} or —NR^{a}C₂₋₆alkOR^{a};

R⁶ is, at each instance, independently selected from H, halo, OR^{a}, C₁₋₆alk, or CF₃;

R⁷ and R⁸ are independently selected from H or C₁₋₆alk, or R⁷ and R⁸, together with the carbon atom to which they are attached, join to form a 3 to 7 membered cycloalkyl ring or a 3-7 membered heterocyclyl ring that includes 1 or 2 heteroatoms selected from O, N, or S;

R⁹ and R¹⁰ are, at each instance, independently selected from H or C₁₋₆alk;

R^{a} is independently, at each instance, H or R^{b}; and

R^{b} is independently, at each instance, phenyl, benzyl or C₁₋₆alk, the phenyl, benzyl and C₁₋₆alk being substituted by 0, 1, 2 or 3 substituents selected from halo, oxo, C₁₋₄alk, C₁₋₃haloalk, —OC₁₋₄alk, —OH, —NH₂, —OC₁₋₄alk, —OC₁₋₄haloalk, —NHC₁₋₄alk, and —N(C₁₋₄alk)C₁₋₄alk.

In some such embodiments, the compound is not one of the following compounds and is not a salt of one of the following compounds:

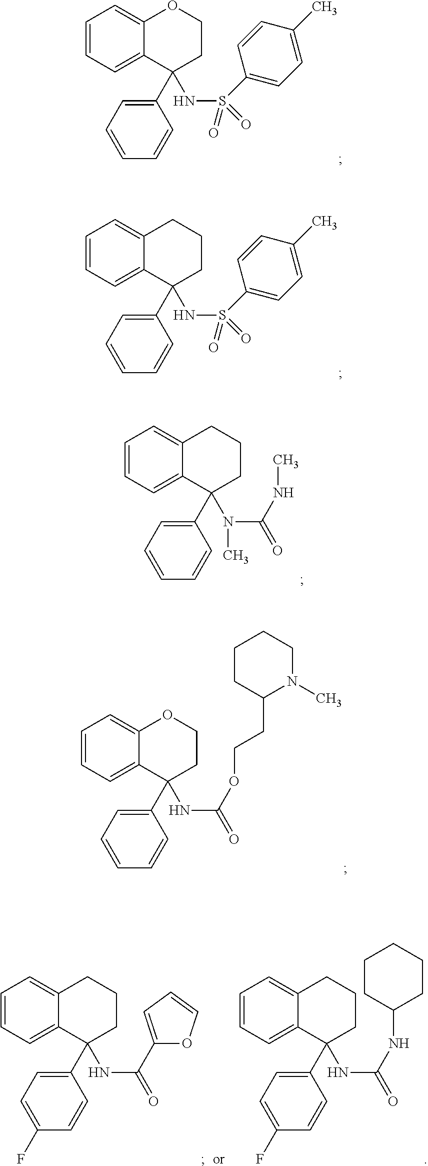

In some embodiments, Y is selected from —O—, —CH₂—, —NH—, or —NR^{1b}—. In still further such embodiments, Y is selected from —O— or —CH₂—.

In another aspect, the invention provides compounds of Formula I or a pharmaceutically-acceptable salt thereof, a tautomer thereof, a pharmaceutically-acceptable salt of the tautomer, a stereoisomer thereof, or a mixture thereof. Compounds of Formula I have the following structure

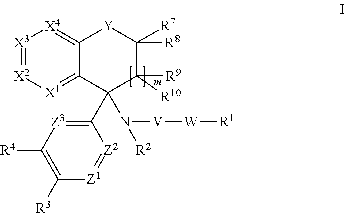

where

V is selected from —C(═O)— or —S(═O)₂—;

W is absent or is selected from —NH—, —NR^{1a}—, or O;

X¹ is selected from —CR⁵— or —N—;

X² is selected from —CR⁵— or —N—;

X³ is selected from —CR⁵— or —N—;

X⁴ is selected from —CR⁵— or —N—;

Y is selected from —O—, —CH₂—, —NH—, —NR^{1b}—, —CF₂—, —C(═O)—, —C(H)(F)—, or —C(H)(OH)—;

Z¹ is selected from —CR⁶— or —N—;

Z² is selected from —CR⁶— or —N—;

Z³ is selected from —CR⁶— or —N—;

wherein 0, 1, or 2 of X¹, X², X³, and X⁴ are N;

wherein 0, 1, or 2 of Z¹, Z², and Z³ are N;

m is 0, 1, or 2;

R¹ is C₁₋₆alk or a direct-bonded, C₁₋₂alk-linked, C₁₋₂alkO-linked, saturated, partially-saturated or unsaturated 3-, 4-, 5-, 6- or 7-membered monocyclic or 7-, 8-, 9-, 10- or 11-membered bicyclic ring containing 0, 1, 2, 3 or 4 heteroatoms selected from N, O and S, but containing no more than one O or S atom, the C₁₋₆alk and ring being substituted by 0, 1, 2 or 3 substituents independently selected from halo, oxo, C₁₋₆alk, C₁₋₆alkOH, C₁₋₆alkOH substituted by 1, 2, or 3 halo substituents, C₁₋₆alk-C(═O)R^{a}, C₁₋₆alk-C(═O)OR^{a}, C₁₋₄haloalk, cyano, nitro, —C(═O)R^{a}, —C(═O)OR^{a}, —C(═O)NR^{a}R^{a}, —C(═O)NR^{a}S(═O)₂R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, ═S, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the ring is additionally substituted by 0 or 1 directly bonded, SO₂ linked, C(═O) linked or CH₂ linked saturated, partially-saturated or unsaturated 3-, 4-, 5-, 6- or 7-membered monocyclic ring containing 0, 1, 2, 3 or 4 heteroatoms selected from N, O and S, but containing no more than one O or S atom, and substituted by 0, 1, 2 or 3 groups selected from halo, oxo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —SR^{a}, —S(═O)R^{a}, —S(═O)₂R^{a}, —S(═O)₂NR^{a}R^{a}, —NR^{a}R^{a}, and —N(R^{a})C(═O)R^{a};

R^{1a} is C₁₋₆alk;

R^{1b} is C₁₋₆alk or —C(═O)R^{b};

R² is H or C₁₋₆alk;

R³ is H, C₁₋₈alk, C₁₋₈alkOH, C₁₋₄haloalk, halo, cyano, R^{b}, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} or —NR^{a}C₂₋₆alkOR^{a};

R⁴ is H, C₁₋₆alk, —C₁₋₃haloalk, —OC₁₋₆alk, —OC₁₋₃haloalk, —N(C₁₋₆alk)C₁₋₆alk, —NHC₁₋₆alk, —NC(═O)C₁₋₆alk, —N(C₁₋₆alk)C₁₋₆alk, F, Cl, Br, CN, OH or NH₂; or R³ and R⁴ together form a four-atom unsaturated bridge containing 0 or 1 N atoms, wherein the bridge is substituted by 0, 1 or 2 R⁵ substituents;

R⁵ is, at each instance, independently selected from H, C₁₋₈alk, C₁₋₈alkOH, C₁₋₄haloalk, halo, cyano, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} or —NR^{a}C₂₋₆alkOR^{a};

R⁶ is, at each instance, independently selected from H, halo, ORE, C₁₋₆alk, or CF₃;

R⁷ and R⁸ are independently selected from H or C₁₋₆alk, or R⁷ and R⁸, together with the carbon atom to which they are attached, join to form a 3 to 7 membered cycloalkyl ring or a 3-7 membered heterocyclyl ring that includes 1 or 2 heteroatoms selected from O, N, or S;

R⁹ and R¹⁶ are, at each instance, independently selected from H or C₁₋₆alk;

R^{a} is independently, at each instance, H or R^{b}; and

R^{b} is independently, at each instance, phenyl, benzyl or C₁₋₆alk, and when R³ is R^{b}, R^{b} may additionally be an unsaturated 5 or 6-membered monocyclic ring containing 1, 2, or 3 heteroatoms selected from N, O, and S, the phenyl, benzyl C₁₋₆alk, and unsaturated 5 or 6-membered monocyclic ring being substituted by 0, 1, 2 or 3 substituents selected from halo, cyano, oxo, C₁₋₄alk, C₁₋₄alkOH, C₁₋₃haloalk, —OC₁₋₄alk, —OH, —NH₂, —OC₁₋₄alk, —OC₁₋₄haloalk, —S(═O)₂C₁₋₄alk, —NHC(═O)—C₁₋₄alk, —C(═O)NH₂, —C(═O)NHC₁₋₄alk, —C(═O)N(C₁₋₄alk)₂, —NHC₁₋₄alk, and —N(C₁₋₄alk)C₁₋₄alk, or a saturated, partially-saturated, or unsaturated 5 or 6-membered monocyclic ring containing 1 or 2 heteroatoms selected from N, O, and S.

In some such embodiments, the compound is not one of the following compounds and is not a salt of one of the following compounds:

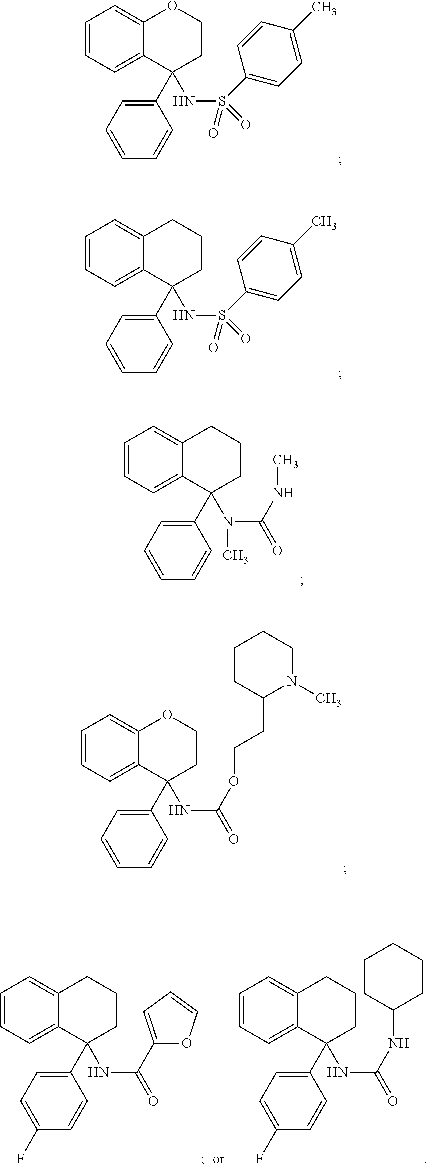

In some embodiments, Y is selected from —O—, —CH₂—, —NH—, or —NR^{1b}—. In still further such embodiments, Y is selected from —O— or —CH₂—.

In some embodiments of the compound of Formula I, the compound of Formula I has the Formula II:

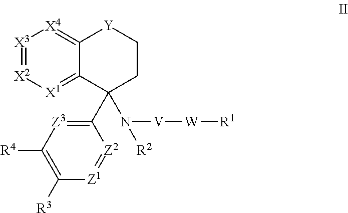

where:

Y is selected from —O— or —CH₂—.

In other embodiments, the compound of Formula II, has the Formula IIA:

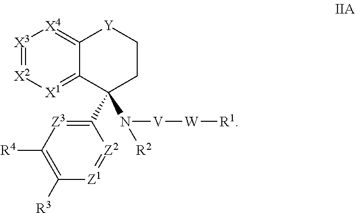

In other embodiments, the compound of Formula I has the Formula III

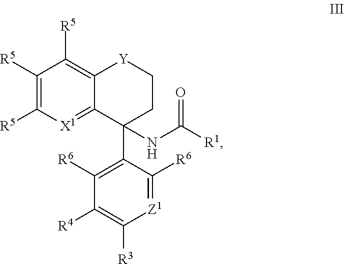

where Y is —O— or —CH₂—.

In yet other embodiments, the compound of Formula III has the Formula IIIA, IIIB, IIIC, or IIID

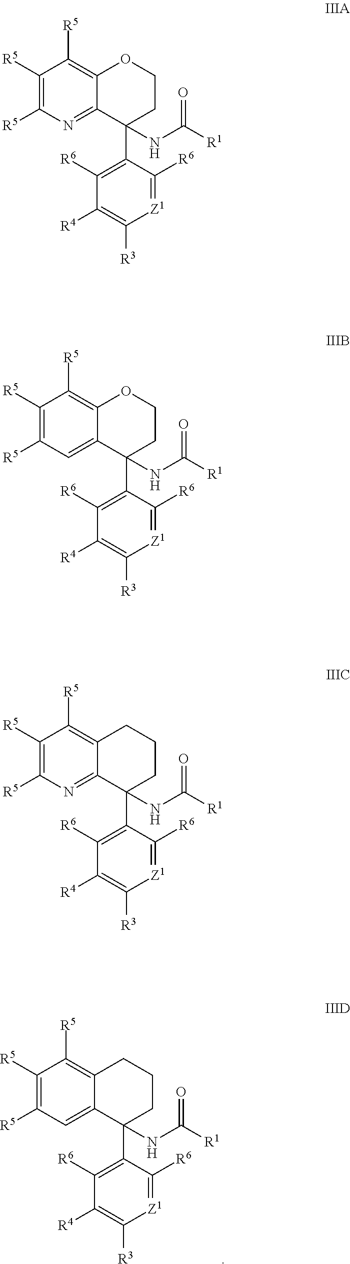

In still other embodiments, the compound of Formula I has the Formula IV

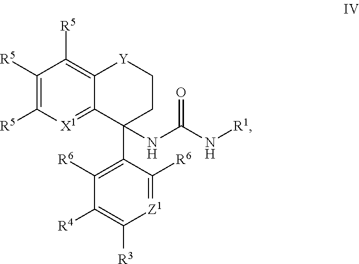

where Y is —O— or —CH₂—.

In some embodiments, the compound of Formula IV has the Formula IVA, IVB, IVC, or IVD

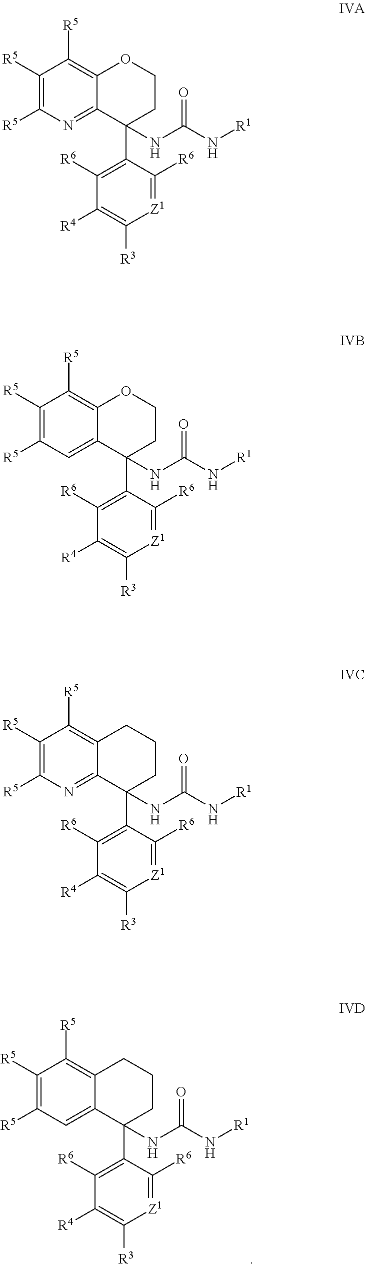

In another aspect, the invention provides pharmaceutical compositions that include the compound of any of the embodiments or the pharmaceutically-acceptable salt thereof, the tautomer thereof, the pharmaceutically-acceptable salt of the tautomer, or the mixture thereof, and a pharmaceutically-acceptable diluent or carrier.

In yet another aspect, the invention provides methods of treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders in a subject. Such methods typically include administering the compound according to any of the embodiments or the pharmaceutically-acceptable salt thereof, the tautomer thereof, the pharmaceutically-acceptable salt of the tautomer, or the mixture thereof to the subject. In some such embodiments, the subject is suffering from neuropathic pain whereas in other embodiments the subject is suffering from migraines or migraine pain.

The compounds of the invention may also be used to prepare pharmaceutical compositions and medicaments. Therefore, in some embodiments, the invention provides the use of the compound according to any of the embodiments or the pharmaceutically-acceptable salt thereof, the tautomer thereof, the pharmaceutically-acceptable salt of the tautomer, or the mixture thereof in the preparation of a medicament.

In another aspect, the invention provides the use of the compound according to any of the embodiments or the pharmaceutically-acceptable salt thereof, the tautomer thereof, the pharmaceutically-acceptable salt of the tautomer, or the mixture thereof for treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders in a subject. In some such embodiments, the compound is used to treat neuropathic pain. In other embodiments, the compound is used to treat migraines or migraine pain

The foregoing merely summarizes certain aspects of the invention and is not intended, nor should it be construed, as limiting the invention in any way. All patents, patent applications and other publications recited herein are hereby incorporated by reference in their entirety.

### DETAILED DESCRIPTION OF THE INVENTION

The compounds of this invention may have in general several asymmetric centers and are typically depicted in the form of racemic mixtures. This invention is intended to encompass racemic mixtures, partially racemic mixtures and separate enantiomers and diastereomers.

Unless otherwise specified, the following definitions apply to terms found in the specification and claims:

“C_{α-β}alk” means an alkyl group comprising a minimum of α and a maximum of β carbon atoms in a branched, cyclical or linear relationship or any combination of the three, wherein α and β represent integers. The alkyl groups described in this section may also contain one or two double or triple bonds. A designation of C₀alk indicates a direct bond. Examples of C₁₋₆alkyl include, but are not limited to the following:

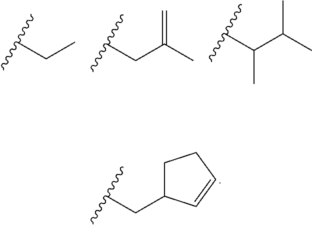

Where the term “C_{α-β}alkyl” and “C_{α-β}cycloalkyl” are used, they relate to acyclic saturated alkyls and cyclic saturated alkyls, respectively.

“Benzo group”, alone or in combination, means the divalent radical C₄H₄═, one representation of which is —CH═CH—CH═CH—, that when vicinally attached to another ring forms a benzene-like ring—for example tetrahydronaphthalene, indole and the like.

The terms “oxo” and “thioxo” represent the groups ═O (as in carbonyl) and ═S (as in thiocarbonyl), respectively.

The term “cyano” refers to a nitrile group which may be written as —C≡N.

“Halo” or “halogen” means a halogen atoms selected from F, Cl, Br and I.

“C_{v-w}haloalk” means an alk group, as described above, wherein any number, but at least one, of the hydrogen atoms attached to the alk chain are replaced by F, Cl, Br or I.

The group N(R^{a})R^{a} and the like include substituents where the two R^{a} groups together form a ring, optionally including a N, O or S atom, and include groups such as:

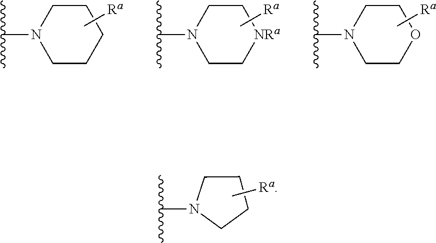

The group N(C_{α-β}alk)C_{α-β}alk, wherein α and β are as defined above, include substituents where the two C_{α-β}alk groups together form a ring, optionally including a N, O or S atom, and include groups such as:

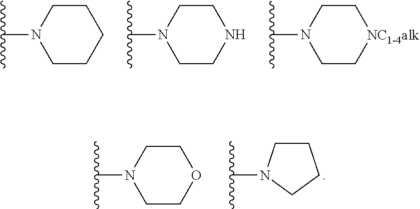

“Heterocycle” means a ring comprising at least one carbon atom and at least one other atom selected from N, O and S. Examples of heterocycles that may be found in the claims include, but are not limited to, the following:

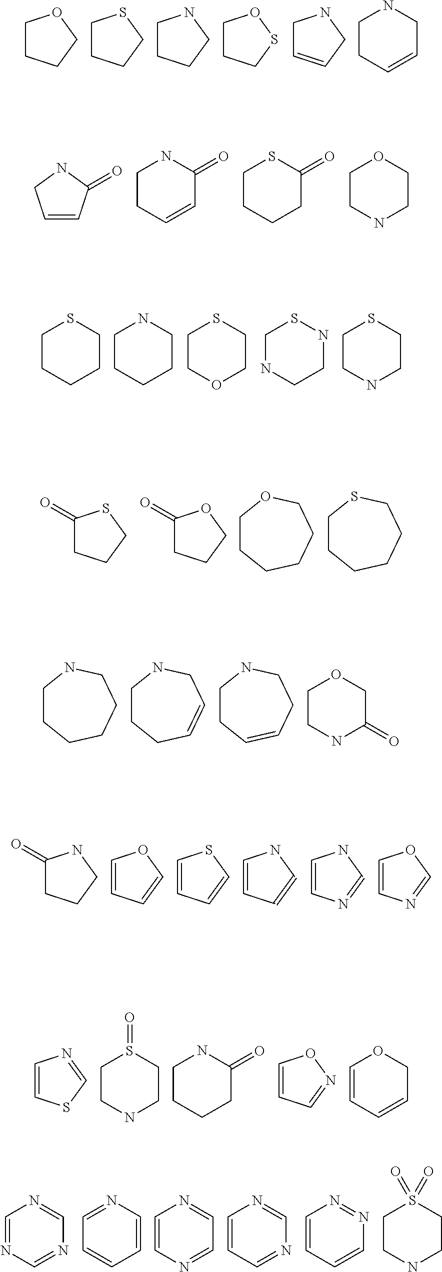

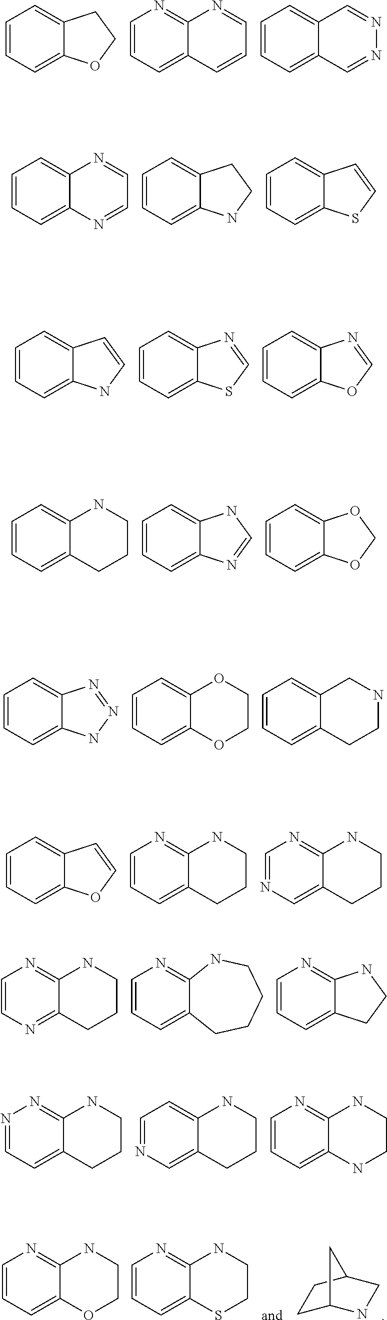

“Pharmaceutically-acceptable salt” means a salt prepared by conventional means, and are well known by those skilled in the art. The “pharmacologically acceptable salts” include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al., J. Pharm. Sci. 66:1 (1977).

“Saturated, partially-saturated or unsaturated” includes substituents saturated with hydrogens, substituents completely unsaturated with hydrogens and substituents partially saturated with hydrogens.

“Leaving group” generally refers to groups readily displaceable by a nucleophile, such as an amine, a thiol or an alcohol nucleophile. Such leaving groups are well known in the art. Examples of such leaving groups include, but are not limited to, N-hydroxysuccinimide, N-hydroxybenzotriazole, halides, triflates, tosylates and the like. Preferred leaving groups are indicated herein where appropriate.

“Protecting group” generally refers to groups well known in the art which are used to prevent selected reactive groups, such as carboxy, amino, hydroxy, mercapto and the like, from undergoing undesired reactions, such as nucleophilic, electrophilic, oxidation, reduction and the like. Preferred protecting groups are indicated herein where appropriate. Examples of amino protecting groups include, but are not limited to, aralkyl, substituted aralkyl, cycloalkenylalkyl and substituted cycloalkenyl alkyl, allyl, substituted allyl, acyl, alkoxycarbonyl, aralkoxycarbonyl, silyl and the like. Examples of aralkyl include, but are not limited to, benzyl, ortho-methylbenzyl, trityl and benzhydryl, which can be optionally substituted with halogen, alkyl, alkoxy, hydroxy, nitro, acylamino, acyl and the like, and salts, such as phosphonium and ammonium salts. Examples of aryl groups include phenyl, naphthyl, indanyl, anthracenyl, 9-(9-phenylfluorenyl), phenanthrenyl, durenyl and the like. Examples of cycloalkenylalkyl or substituted cycloalkylenylalkyl radicals, preferably have 6-10 carbon atoms, include, but are not limited to, cyclohexenyl methyl and the like. Suitable acyl, alkoxycarbonyl and aralkoxycarbonyl groups include benzyloxycarbonyl, t-butoxycarbonyl, iso-butoxycarbonyl, benzoyl, substituted benzoyl, butyryl, acetyl, trifluoroacetyl, trichloro acetyl, phthaloyl and the like. A mixture of protecting groups can be used to protect the same amino group, such as a primary amino group can be protected by both an aralkyl group and an aralkoxycarbonyl group Amino protecting groups can also form a heterocyclic ring with the nitrogen to which they are attached, for example, 1,2-bis(methylene)benzene, phthalimidyl, succinimidyl, maleimidyl and the like and where these heterocyclic groups can further include adjoining aryl and cycloalkyl rings. In addition, the heterocyclic groups can be mono-, di- or tri-substituted, such as nitrophthalimidyl Amino groups may also be protected against undesired reactions, such as oxidation, through the formation of an addition salt, such as hydrochloride, toluenesulfonic acid, trifluoroacetic acid and the like. Many of the amino protecting groups are also suitable for protecting carboxy, hydroxy and mercapto groups. For example, aralkyl groups. Alkyl groups are also suitable groups for protecting hydroxy and mercapto groups, such as tert-butyl.

Silyl protecting groups are silicon atoms optionally substituted by one or more alkyl, aryl and aralkyl groups. Suitable silyl protecting groups include, but are not limited to, trimethylsilyl, triethylsilyl, triisopropylsilyl, tert-butyldimethylsilyl, dimethylphenylsilyl, 1,2-bis(dimethylsilyl)benzene, 1,2-bis(dimethylsilyl)ethane and diphenylmethylsilyl. Silylation of an amino groups provide mono- or di-silylamino groups. Silylation of aminoalcohol compounds can lead to a N,N,O-trisilyl derivative. Removal of the silyl function from a silyl ether function is readily accomplished by treatment with, for example, a metal hydroxide or ammonium fluoride reagent, either as a discrete reaction step or in situ during a reaction with the alcohol group. Suitable silylating agents are, for example, trimethylsilyl chloride, tert-butyl-dimethylsilyl chloride, phenyldimethylsilyl chloride, diphenylmethyl silyl chloride or their combination products with imidazole or DMF. Methods for silylation of amines and removal of silyl protecting groups are well known to those skilled in the art. Methods of preparation of these amine derivatives from corresponding amino acids, amino acid amides or amino acid esters are also well known to those skilled in the art of organic chemistry including amino acid/amino acid ester or aminoalcohol chemistry.

Protecting groups are removed under conditions which will not affect the remaining portion of the molecule. These methods are well known in the art and include acid hydrolysis, hydrogenolysis and the like. A preferred method involves removal of a protecting group, such as removal of a benzyloxycarbonyl group by hydrogenolysis utilizing palladium on carbon in a suitable solvent system such as an alcohol, acetic acid, and the like or mixtures thereof. A t-butoxycarbonyl protecting group can be removed utilizing an inorganic or organic acid, such as HCl or trifluoroacetic acid, in a suitable solvent system, such as dioxane or methylene chloride. The resulting amino salt can readily be neutralized to yield the free amine Carboxy protecting group, such as methyl, ethyl, benzyl, tert-butyl, 4-methoxyphenylmethyl and the like, can be removed under hydrolysis and hydrogenolysis conditions well known to those skilled in the art.

Various compounds of the invention contain one or more chiral centers, and can exist as racemic mixtures of enantiomers, mixtures of diastereomers or enantiomerically or optically pure compounds. This invention encompasses the use of stereomerically pure forms of such compounds, as well as the use of mixtures of those forms. For example, mixtures comprising equal or unequal amounts of the enantiomers of a particular compound of the invention may be used in methods and compositions of the invention. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al. (1997) Tetrahedron 33:2725; Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

It should be noted that compounds of the invention may contain groups that may exist in tautomeric forms, such as cyclic and acyclic amidine and guanidine groups, heteroatom substituted heteroaryl groups (Y′=O, S, NR), and the like, which are illustrated in the following examples:

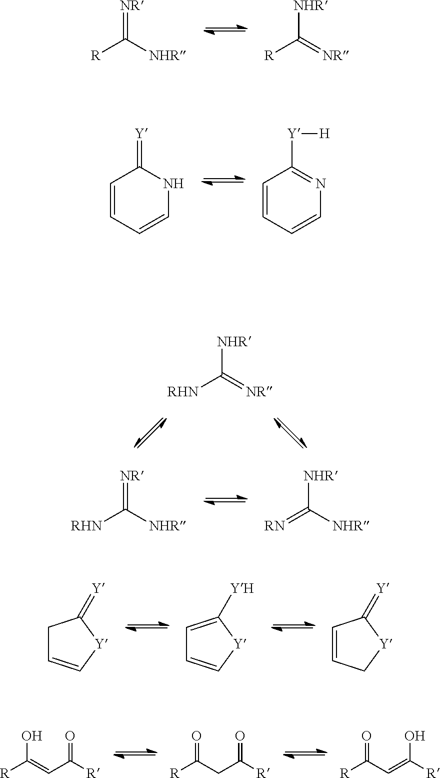

and though one form is named, described, displayed and/or claimed herein, all the tautomeric forms are intended to be inherently included in such name, description, display and/or claim.

Prodrugs of the compounds of this invention are also contemplated by this invention. A prodrug is an active or inactive compound that is modified chemically through in vivo physiological action, such as hydrolysis, metabolism and the like, into a compound of this invention following administration of the prodrug to a patient. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use.

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). Radiolabeled compounds are useful as therapeutic or prophylactic agents, research reagents, e.g., GPR⁴⁰ assay reagents, and diagnostic agents, e.g., in vivo imaging agents. All isotopic variations of the compounds of the invention, whether radioactive or not, are intended to be encompassed within the scope of the invention. For example, if a variable is said to be H, this means that variable may also be deuterium (D) or tritium (T).

The terms “treat”, “treating” and “treatment”, as used herein, are meant to include alleviating or abrogating a condition or disease and/or its attendant symptoms. In some instances treating may also involve prevention of symptoms. The terms “prevent”, “preventing” and “prevention”, as used herein, refer to a method of delaying or precluding the onset of a condition or disease and/or its attendant symptoms, barring a subject from acquiring a condition or disease, or reducing a subject's risk of acquiring a condition or disease.

The term “therapeutically effective amount” refers to that amount of the compound that will elicit the biological or medical response of a tissue, system, or subject that is being sought. The term “therapeutically effective amount” includes that amount of a compound that, when administered, is sufficient to prevent development of, or alleviate to some extent, one or more of the symptoms of the condition or disorder being treated in a subject. The therapeutically effective amount in a subject will vary depending on the compound, the disease and its severity, and the age, weight, etc., of the subject to be treated. Typically, a therapeutically effective amount of a compound or a salt thereof is administered to subjects in various method regimens. In some embodiments, a therapeutically effective amount of a compound is administered to a subject prior to the onset of a migraine or at the first indication that a migraine may be about to occur or occurring.

The term “subject” is defined herein to include animals such as mammals, including, but not limited to, primates (e.g., humans), cows, sheep, goats, horses, dogs, cats, rabbits, rats, mice and the like. In preferred embodiments, the subject is a human.

“Pharmaceutically-acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, salicylic acid, benzoic acid, 3-(4-hydroxybenzoyl)benzoic acid, cinnamic acid, mandelic acid, methanesulfonic acid, ethanesulfonic acid, benzenesulfonic acid, p-toluenesulfonic acid, ethane disulfonic acid and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like. Several salt forms may exist as hydrates such that the use of the term salt is generally defined to include hydrated and non-hydrated forms of the salt.

The specification and claims contain listing of species using the language like “selected from . . . and .” and “is . . . or .” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups as needed.

As used herein, the terms “comprising” and “including” and other forms of these words are used herein in their open, non-limiting sense. For example, if a composition is said to comprise A, B, and C, then A, B, and C are in the composition, but D, E, and/or F may be in the composition as well.

Another aspect of the invention relates to a method of treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders, comprising the step of administering a compound as described above.

Another aspect of the invention relates to a pharmaceutical composition comprising a compound according to claim 1 and a pharmaceutically-acceptable diluent or carrier.

Another aspect of the invention relates to the use of a compound according to any of the above embodiments as a medicament.

Another aspect of the invention relates to the use of a compound according to any of the above embodiments in the manufacture of a medicament for the treatment of acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, anxiety, depression, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders.

### EMBODIMENTS

The embodiments listed below are presented in numbered form for convenience.

1. In a first embodiment, the invention provides a compound of Formula I having the following structure:

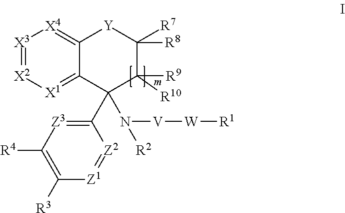

a pharmaceutically-acceptable salt thereof, a tautomer thereof, a pharmaceutically-acceptable salt of the tautomer, a stereoisomer thereof, or a mixture thereof, wherein:

V is selected from —C(═O)— or —S(═O)₂—;

W is absent or is selected from —NH—, —NR^{1a}—, or O;

X¹ is selected from —CR⁵— or —N—;

X² is selected from —CR⁵— or —N—;

X³ is selected from —CR⁵— or —N—;

X⁴ is selected from —CR⁵— or —N—;

Y is selected from —O—, —CH₂—, —NH—, —NR^{1b}—, —CF₂—, —C(═O)—, —C(H)(F)—, or —C(H)(OH)—;

Z¹ is selected from —CR⁶— or —N—;

Z² is selected from —CR⁶— or —N—;

Z³ is selected from —CR⁶— or —N—;

wherein 0, 1, or 2 of X¹, X², X³, and X⁴ are N;

wherein 0, 1, or 2 of Z¹, Z², and Z³ are N;

m is 0, 1, or 2;

R¹ is C₁₋₆alk or a direct-bonded, C₁₋₂alk-linked, C₁₋₂alkO-linked, saturated, partially-saturated or unsaturated 3-, 4-, 5-, 6- or 7-membered monocyclic or 7-, 8-, 9-, 10- or 11-membered bicyclic ring containing 0, 1, 2, 3 or 4 heteroatoms selected from N, O and S, but containing no more than one O or S atom, the C₁₋₆alk and ring being substituted by 0, 1, 2 or 3 substituents independently selected from halo, oxo, C₁₋₆alk, C₁₋₆alkOH, C₁₋₆alk-C(═O)R^{a}, C₁₋₆alk-C(═O)OR^{a}, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, ═S, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the ring is additionally substituted by 0 or 1 directly bonded, SO₂ linked, C(═O) linked or CH₂ linked saturated, partially-saturated or unsaturated 3-, 4-, 5-, 6- or 7-membered monocyclic ring containing 0, 1, 2, 3 or 4 heteroatoms selected from N, O and S, but containing no more than one O or S atom, and substituted by 0, 1, 2 or 3 groups selected from halo, oxo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —SR^{a}, —S(═O)R^{a}, —S(═O)₂R^{a}, —S(═O)₂NR^{a}R^{a}, —NR^{a}R^{a}, and —N(R^{a})C(═O)R^{a};

R^{1a} is C₁₋₆alk;

R^{1b} is C₁₋₆alk or —C(═O)R^{b};

R² is H or C₁₋₆alk;

R³ is H, C₁₋₈alk, C₁₋₈alkOH, C₁₋₄haloalk, halo, cyano, R^{b}, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} or —NR^{a}C₂₋₆alkOR^{a};

R⁴ is H, C₁₋₆alk, —C₁₋₃haloalk, —OC₁₋₆alk, —OC₁₋₃haloalk, —N(C₁₋₆alk)C₁₋₆alk, —NHC₁₋₆alk, —NC(═O)C₁₋₆alk, —N(C₁₋₆alk)C₁₋₆alk, F, Cl, Br, CN, OH or NH₂; or R³ and R⁴ together form a four-atom unsaturated bridge containing 0 or 1 N atoms, wherein the bridge is substituted by 0, 1 or 2 R⁵ substituents;

R⁵ is, at each instance, independently selected from H, C₁₋₈alk, C₁₋₈alkOH, C₁₋₄haloalk, halo, cyano, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SRa, —S(═O)R^{b}, —S(═O)₂R^{b}, —S(═O)₂NR^{a}R^{a}, —NR^{a}R^{a}, —N(R^{a})C(═O)R^{b}, —N(Ra)C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} or —NR^{a}C₂₋₆alkOR^{a};

R⁶ is, at each instance, independently selected from H, halo, OR^{a}, C₁₋₆alk, or CF₃;

R⁷ and R⁸ are independently selected from H or C₁₋₆alk, or R⁷ and R⁸, together with the carbon atom to which they are attached, join to form a 3 to 7 membered cycloalkyl ring or a 3-7 membered heterocyclyl ring that includes 1 or 2 heteroatoms selected from O, N, or S;

R⁹ and R¹⁰ are, at each instance, independently selected from H or C₁₋₆alk;

R^{a} is independently, at each instance, H or R^{b}; and

R^{b} is independently, at each instance, phenyl, benzyl or C₁₋₆alk, the phenyl, benzyl and C₁₋₆alk being substituted by 0, 1, 2 or 3 substituents selected from halo, oxo, C₁₋₄alk, C₁₋₃haloalk, —OC₁₋₄alk, —OH, —NH₂, —OC₁₋₄alk, —OC₁₋₄haloalk, —NHC₁₋₄alk, and —N(C₁₋₄alk)C₁₋₄alk;

wherein the compound is not one of the following compounds and is not a salt thereof:

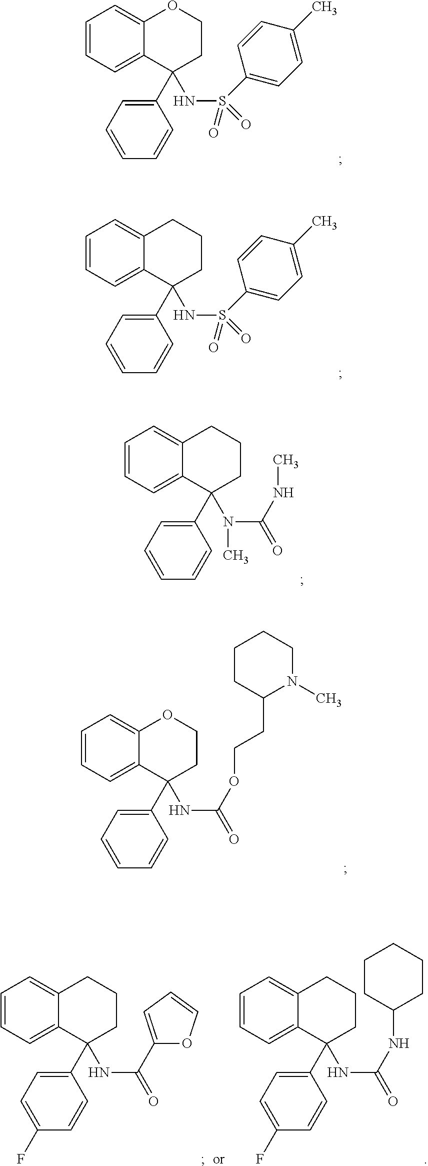

In some versions of embodiment 1, the compound is not one of the following compounds, is not a salt thereof, is not a tautomer thereof, is not a salt of a tautomer, is not a stereoisomer thereof, and is not a salt of a stereoisomer:

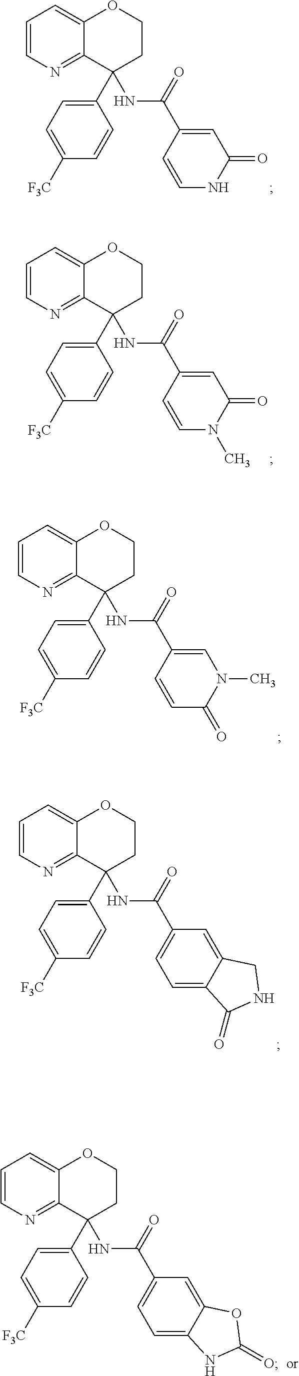 [SMILES: O=C(NC1(c2ccc(C(F)(F)F)cc2)CCOc2cccnc21)c1cc[nH]c(=O)c1 ; Cn1ccc(C(=O)NC2(c3ccc(C(F)(F)F)cc3)CCOc3cccnc32)cc1=O ; Cn1cc(C(=O)NC2(c3ccc(C(F)(F)F)cc3)CCOc3cccnc32)ccc1=O ; O=C(NC1(c2ccc(C(F)(F)F)cc2)CCOc2cccnc21)c1ccc2c(c1)CNC2=O ; O=C(NC1(c2ccc(C(F)(F)F)cc2)CCOc2cccnc21)c1ccc2[nH]c(=O)oc2c1]

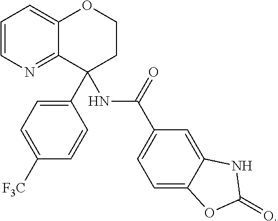 [SMILES: O=C(NC1(c2ccc(C(F)(F)F)cc2)CCOc2cccnc21)c1ccc2oc(=O)[nH]c2c1]

In some embodiments, Y is selected from —O—, —CH₂—, —NH—, or —NR^{1b}—. In still further such embodiments, Y is selected from —O— or —CH₂—.

In some embodiments, X¹ is N; X², X³ and X⁴ are CR⁵; Y is O; m is 1; R⁷, R⁸, R⁹, and R¹⁰ are H; two or three of Z¹, Z², and Z³ are CR⁶; R² is H; V is —C(═O)—; W is absent or is —NH—; R³ is —CF₃ or —OCF₃; R⁴ is —F, —Cl, or —OCF₃; and R¹ is a partially-saturated or unsaturated 3-, 4-, 5-, 6- or 7-membered monocyclic or 7-, 8-, 9-, 10- or 11-membered bicyclic ring containing 0, 1, 2, 3 or 4 heteroatoms selected from N, O and S, but containing no more than one O or S atom, and the ring is substituted by 0, 1, 2 or 3 substituents independently selected from halo, oxo, C₁₋₆alk, C₁₋₆ alkOH, C₁₋₆ alk-C(═O)R^{a}, C₁₋₆ alk-C(═O)OR^{a}, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, ═S, —S(═O)Ra, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(Ra)C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the ring is additionally substituted by 0 or 1 directly bonded, SO₂ linked, C(═O) linked or CH₂ linked saturated, partially-saturated or unsaturated 3-, 4-, 5-, 6- or 7-membered monocyclic ring containing 0, 1, 2, 3 or 4 heteroatoms selected from N, O and S, but containing no more than one O or S atom, and substituted by 0, 1, 2 or 3 groups selected from halo, oxo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —SR^{a}, —S(═O)R^{a}, —S(═O)₂R^{a}, —S(═O)₂NR^{a}R^{a}, —NR^{a}R^{a}, and —N(R^{a})C(═O)R^{a}. In some such embodiments, R⁵ is H; Z¹, Z², and Z³ are each CR⁶; W is absent; and R⁴ is —F. In still further such embodiments, R³ is —CF₃ whereas in other such embodiments, R³ is —OCF₃. In still further such embodiments, R⁶ is H.

2. The compound of embodiment 1 or the pharmaceutically-acceptable salt thereof, the tautomer thereof, the pharmaceutically-acceptable salt of the tautomer, the stereoisomer thereof, or the mixture thereof, wherein R⁷ and R⁸ are both H.

3. The compound of embodiment 1 or the pharmaceutically-acceptable salt thereof, the tautomer thereof, the pharmaceutically-acceptable salt of the tautomer, the stereoisomer thereof, or the mixture thereof, wherein R⁷ and R⁸ are selected from H and —CH₃.

4. The compound of embodiment 3 or the pharmaceutically-acceptable salt thereof, the tautomer thereof, the pharmaceutically-acceptable salt of the tautomer, the stereoisomer thereof, or the mixture thereof, wherein R⁷ and R⁸ are both —CH₃.

5. The compound of embodiment 1 or the pharmaceutically-acceptable salt thereof, the tautomer thereof, the pharmaceutically-acceptable salt of the tautomer, the stereoisomer thereof, or the mixture thereof, wherein R⁷ and R⁸, together with the carbon atom to which they are attached, join to form a 3-7 membered cycloalkyl ring.

6. The compound of embodiment 5 or the pharmaceutically-acceptable salt thereof, the tautomer thereof, the pharmaceutically-acceptable salt of the tautomer, the stereoisomer thereof, or the mixture thereof, wherein R⁷ and R⁸, together with the carbon atom to which they are attached, join to form a cyclopropyl or cyclobutyl ring.

7. The compound of embodiment 1 or the pharmaceutically-acceptable salt thereof, the tautomer thereof, the pharmaceutically-acceptable salt of the tautomer, the stereoisomer thereof, or the mixture thereof, wherein m is 0.

8. The compound of any one of embodiments 1-6 or the pharmaceutically-acceptable salt thereof, the tautomer thereof, the pharmaceutically-acceptable salt of the tautomer, the stereoisomer thereof, or the mixture thereof, wherein m is 1.

9. The compound of any one of embodiment 1-6 or the pharmaceutically-acceptable salt thereof, the tautomer thereof, the pharmaceutically-acceptable salt of the tautomer, the stereoisomer thereof, or the mixture thereof, wherein m is 2.

10. The compound of any one of embodiments 1-9 or the pharmaceutically-acceptable salt thereof, the tautomer thereof, the pharmaceutically-acceptable salt of the tautomer, the stereoisomer thereof, or the mixture thereof, wherein Y is —NH—.

11. The compound of any one of embodiments 1-9 or the pharmaceutically-acceptable salt thereof, the tautomer thereof, the pharmaceutically-acceptable salt of the tautomer, the stereoisomer thereof, or the mixture thereof, wherein Y is —NR^{1b}— and R^{1b} is —CH₃.

12. The compound of any one of embodiments 1-9 or the pharmaceutically-acceptable salt thereof, the tautomer thereof, the pharmaceutically-acceptable salt of the tautomer, the stereoisomer thereof, or the mixture thereof, wherein Y is —O—.

13. The compound of any one of embodiments 1-9 or the pharmaceutically-acceptable salt thereof, the tautomer thereof, the pharmaceutically-acceptable salt of the tautomer, the stereoisomer thereof, or the mixture thereof, wherein Y is —CH₂—.

14. The compound of any one of embodiments 1-13 or the pharmaceutically-acceptable salt thereof, the tautomer thereof, the pharmaceutically-acceptable salt of the tautomer, the stereoisomer thereof, or the mixture thereof, wherein R⁹ and R¹⁰ are H.

15. The compound of embodiment 1 or the pharmaceutically-acceptable salt thereof, the tautomer thereof, the pharmaceutically-acceptable salt of the tautomer, the stereoisomer thereof, or the mixture thereof, wherein the compound of Formula I has the Formula II:

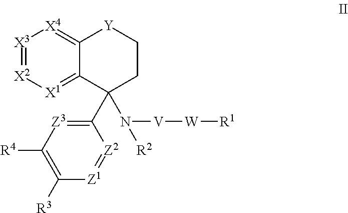

wherein:

Y is selected from —O— or —CH₂—.

16. The compound of embodiment 15 or the pharmaceutically-acceptable salt thereof, the tautomer thereof, the pharmaceutically-acceptable salt of the tautomer, the stereoisomer thereof, or the mixture thereof, wherein the compound of Formula II has the Formula IIA:

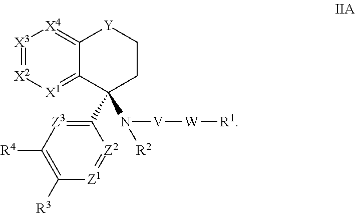

17. The compound of any one of embodiments 1-16 or the pharmaceutically-acceptable salt thereof, the tautomer thereof, the pharmaceutically-acceptable salt of the tautomer, the stereoisomer thereof, or the mixture thereof, wherein 0 or 1 of X¹, X², X³, and X⁴ are N.

18. The compound of embodiment 17 or the pharmaceutically-acceptable salt thereof, the tautomer thereof, the pharmaceutically-acceptable salt of the tautomer, the stereoisomer thereof, or the mixture thereof, wherein 1 of X¹, X², X³, and X⁴ are N.

19. The compound of embodiment 17 or the pharmaceutically-acceptable salt thereof, the tautomer thereof, the pharmaceutically-acceptable salt of the tautomer, the stereoisomer thereof, or the mixture thereof, wherein 0 of X¹, X², X³, and X⁴ are N.

20. The compound of embodiment 17 or the pharmaceutically-acceptable salt thereof, the tautomer thereof, the pharmaceutically-acceptable salt of the tautomer, the stereoisomer thereof, or the mixture thereof, wherein X¹ is N and X², X³, and X⁴ are all —CR⁵—.

21. The compound of embodiment 17 or the pharmaceutically-acceptable salt thereof, the tautomer thereof, the pharmaceutically-acceptable salt of the tautomer, the stereoisomer thereof, or the mixture thereof, wherein X² is N and X¹, X³, and X⁴ are all —CR⁵—.

22. The compound of embodiment 17 or the pharmaceutically-acceptable salt thereof, the tautomer thereof, the pharmaceutically-acceptable salt of the tautomer, the stereoisomer thereof, or the mixture thereof, wherein X³ is N and X¹, X², and X⁴ are all —CR⁵—.

23. The compound of embodiment 17 or the pharmaceutically-acceptable salt thereof, the tautomer thereof, the pharmaceutically-acceptable salt of the tautomer, the stereoisomer thereof, or the mixture thereof, wherein X⁴ is N and X¹, X², and X³ are all —CR⁵—.

24. The compound of any one of embodiments 1-23 or the pharmaceutically-acceptable salt thereof, the tautomer thereof, the pharmaceutically-acceptable salt of the tautomer, the stereoisomer thereof, or the mixture thereof, wherein 0 or 1 of Z¹, Z², and Z³ are N.

25. The compound of embodiment 24 or the pharmaceutically-acceptable salt thereof, the tautomer thereof, the pharmaceutically-acceptable salt of the tautomer, the stereoisomer thereof, or the mixture thereof, wherein 0 of Z¹, Z², and Z³ are N.

26. The compound of embodiment 24 or the pharmaceutically-acceptable salt thereof, the tautomer thereof, the pharmaceutically-acceptable salt of the tautomer, the stereoisomer thereof, or the mixture thereof, wherein 1 of Z¹, Z², and Z³ are N.

27. The compound of embodiment 24 or the pharmaceutically-acceptable salt thereof, the tautomer thereof, the pharmaceutically-acceptable salt of the tautomer, the stereoisomer thereof, or the mixture thereof, wherein Z¹ is N and Z² and Z³ are —CR⁶—.

28. The compound of embodiment 24 or the pharmaceutically-acceptable salt thereof, the tautomer thereof, the pharmaceutically-acceptable salt of the tautomer, the stereoisomer thereof, or the mixture thereof, wherein Z² is N and Z¹ and Z³ are —CR⁶—.

29. The compound of embodiment 24 or the pharmaceutically-acceptable salt thereof, the tautomer thereof, the pharmaceutically-acceptable salt of the tautomer, the stereoisomer thereof, or the mixture thereof, wherein Z³ is N and Z¹ and Z² are —CR⁶—.

30. The compound of any one of embodiments 1-29 or the pharmaceutically-acceptable salt thereof, the tautomer thereof, the pharmaceutically-acceptable salt of the tautomer, the stereoisomer thereof, or the mixture thereof, wherein V is —C(═O)—.

31. The compound of any one of embodiments 1-29 or the pharmaceutically-acceptable salt thereof, the tautomer thereof, the pharmaceutically-acceptable salt of the tautomer, the stereoisomer thereof, or the mixture thereof, wherein V is —S(═O)₂7.

32. The compound of any one of embodiments 1-31 or the pharmaceutically-acceptable salt thereof, the tautomer thereof, the pharmaceutically-acceptable salt of the tautomer, the stereoisomer thereof, or the mixture thereof, wherein W is absent.

33. The compound of any one of embodiments 1-31 or the pharmaceutically-acceptable salt thereof, the tautomer thereof, the pharmaceutically-acceptable salt of the tautomer, the stereoisomer thereof, or the mixture thereof, wherein W is —NH—.

34. The compound of any one of embodiments 1-31 or the pharmaceutically-acceptable salt thereof, the tautomer thereof, the pharmaceutically-acceptable salt of the tautomer, the stereoisomer thereof, or the mixture thereof, wherein W is —O—.

35. The compound of any one of embodiments 1-34 or the pharmaceutically-acceptable salt thereof, the tautomer thereof, the pharmaceutically-acceptable salt of the tautomer, the stereoisomer thereof, or the mixture thereof, wherein R² is H.

36. The compound of embodiment 1 or the pharmaceutically-acceptable salt thereof, the tautomer thereof, the pharmaceutically-acceptable salt of the tautomer, the stereoisomer thereof, or the mixture thereof, wherein the compound of Formula I has the Formula III

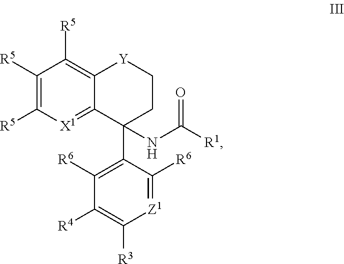

wherein Y is —O— or —CH₂—. In some such embodiments, the compounds has the Formula III′

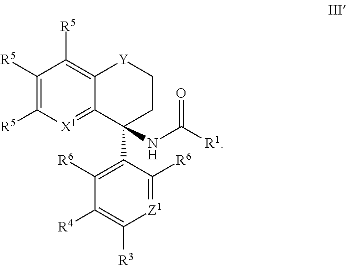

37. The compound of embodiment 36 or the pharmaceutically-acceptable salt thereof, the tautomer thereof, the pharmaceutically-acceptable salt of the tautomer, the stereoisomer thereof, or the mixture thereof, wherein the compound of Formula III has the Formula IIIA, IIIB, IIIC, or IIID

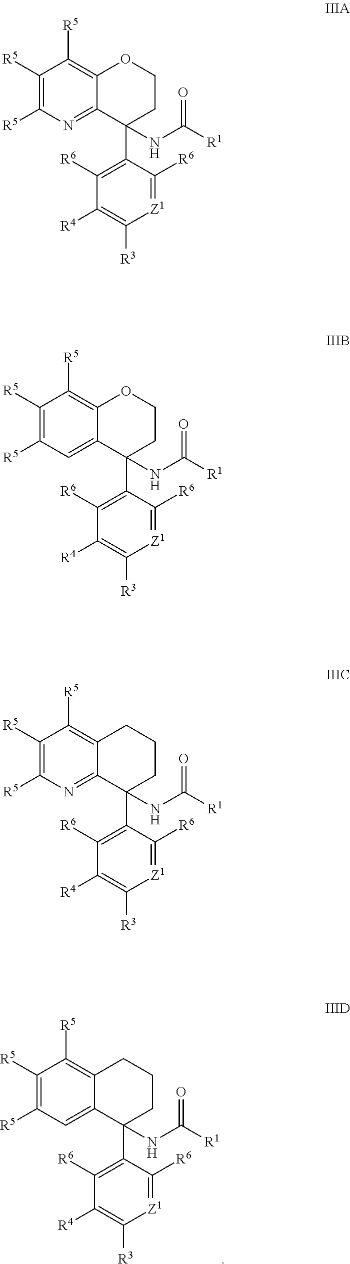

38. The compound of embodiment 37 or the pharmaceutically-acceptable salt thereof, the tautomer thereof, the pharmaceutically-acceptable salt of the tautomer, the stereoisomer thereof, or the mixture thereof, wherein the compound of Formula III has the Formula IIIA. In some such embodiments, the compound has the Formula IIIA′

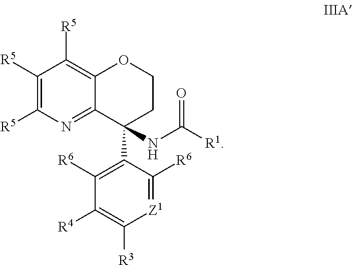

39. The compound of embodiment 37 or the pharmaceutically-acceptable salt thereof, the tautomer thereof, the pharmaceutically-acceptable salt of the tautomer, the stereoisomer thereof, or the mixture thereof, wherein the compound of Formula III has the Formula IIIB. In some such embodiments, the compound has the Formula IIIB′

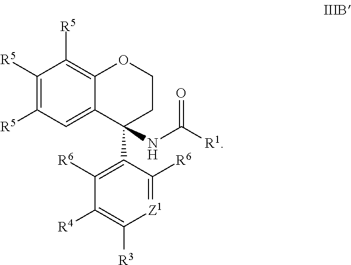

40. The compound of embodiment 37 or the pharmaceutically-acceptable salt thereof, the tautomer thereof, the pharmaceutically-acceptable salt of the tautomer, the stereoisomer thereof, or the mixture thereof, wherein the compound of Formula III has the Formula IIIC. In some such embodiments, the compound has the Formula IIIC′

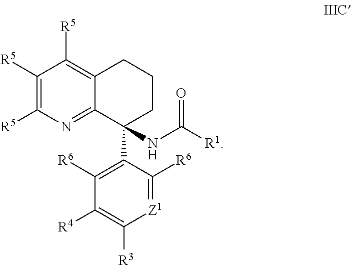

41. The compound of embodiment 37 or the pharmaceutically-acceptable salt thereof, the tautomer thereof, the pharmaceutically-acceptable salt of the tautomer, the stereoisomer thereof, or the mixture thereof, wherein the compound of Formula III has the Formula IIID. In some such embodiments, the compound has the Formula IIID′

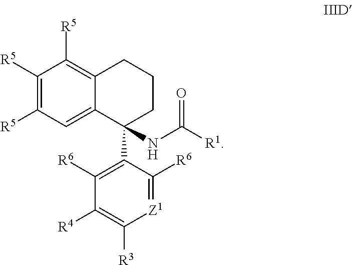

42. The compound of embodiment 1 or the pharmaceutically-acceptable salt thereof, the tautomer thereof, the pharmaceutically-acceptable salt of the tautomer, the stereoisomer thereof, or the mixture thereof, wherein the compound of Formula I has the Formula IV

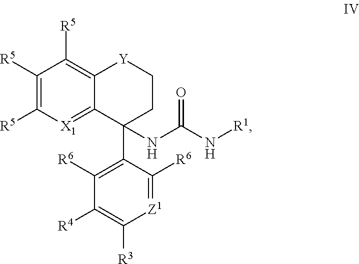

wherein Y is —O— or —CH₂—. In some embodiments, the compound of Formula IV has the Formula IV′

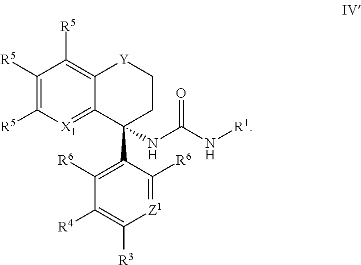

43. The compound of embodiment 42 or the pharmaceutically-acceptable salt thereof, the tautomer thereof, the pharmaceutically-acceptable salt of the tautomer, the stereoisomer thereof, or the mixture thereof, wherein the compound of Formula IV has the Formula IVA, IVB, IVC, or IVD

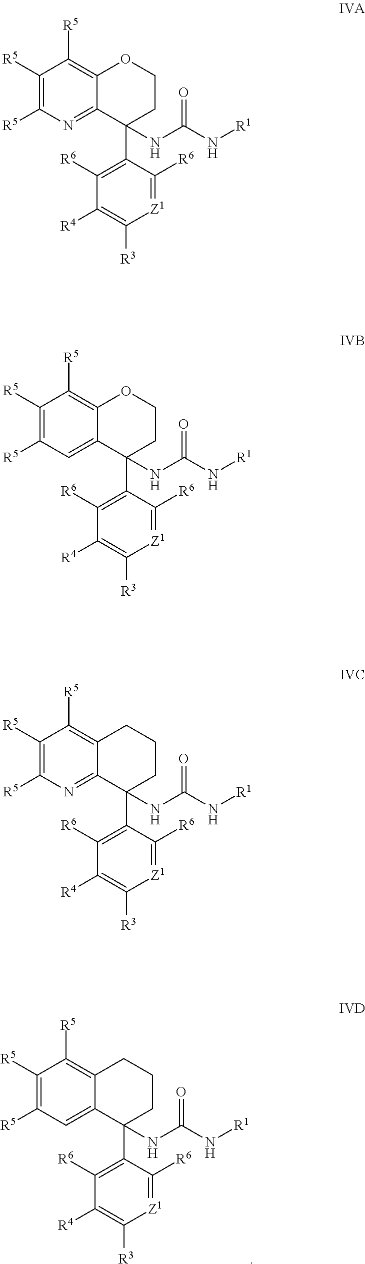

44. The compound of embodiment 43 or the pharmaceutically-acceptable salt thereof, the tautomer thereof, the pharmaceutically-acceptable salt of the tautomer, the stereoisomer thereof, or the mixture thereof, wherein the compound of Formula IV has the Formula IVA. In some such embodiments, the compound has the Formula IVA′

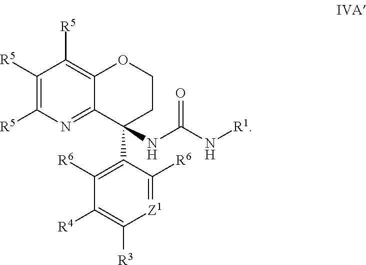

45. The compound of embodiment 43 or the pharmaceutically-acceptable salt thereof, the tautomer thereof, the pharmaceutically-acceptable salt of the tautomer, the stereoisomer thereof, or the mixture thereof, wherein the compound of Formula IV has the Formula IVB. In some such embodiments, the compound has the Formula IVB′

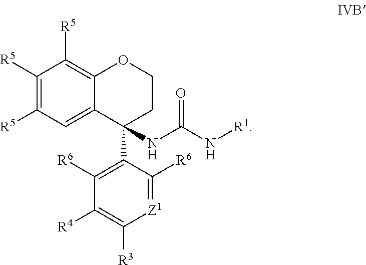

46. The compound of embodiment 43 or the pharmaceutically-acceptable salt thereof, the tautomer thereof, the pharmaceutically-acceptable salt of the tautomer, the stereoisomer thereof, or the mixture thereof, wherein the compound of Formula IV has the Formula IVC. In some such embodiments, the compound has the Formula IVC′

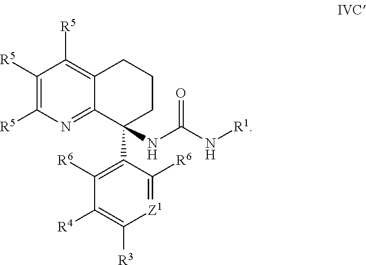

47. The compound of embodiment 43 or the pharmaceutically-acceptable salt thereof, the tautomer thereof, the pharmaceutically-acceptable salt of the tautomer, the stereoisomer thereof, or the mixture thereof, wherein the compound of Formula IV has the Formula IVD. In some such embodiments, the compound has the Formula IVD′

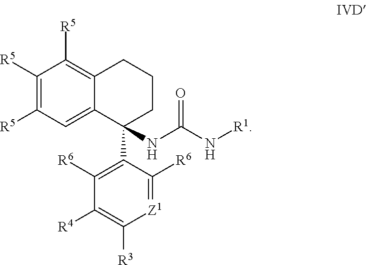

48. The compound of any one of embodiments 1-47 or the pharmaceutically-acceptable salt thereof, the tautomer thereof, the pharmaceutically-acceptable salt of the tautomer, the stereoisomer thereof, or the mixture thereof, wherein R³ is selected from H, C₁₋₈alk, C₁₋₈alkOH, C₁₋₄haloalk, halo, or —OR^{a}.

49. The compound of any one of embodiments 1-47 or the pharmaceutically-acceptable salt thereof, the tautomer thereof, the pharmaceutically-acceptable salt of the tautomer, the stereoisomer thereof, or the mixture thereof, wherein R³ is selected from —H, —CH₃, —F, —Cl, —CF₃, or —OCF₃.

50. The compound of embodiment 49 or the pharmaceutically-acceptable salt thereof, the tautomer thereof, the pharmaceutically-acceptable salt of the tautomer, the stereoisomer thereof, or the mixture thereof, wherein R³ is selected from —CH₃, —F, —Cl, —CF₃, or —OCF₃.

51. The compound of embodiment 49 or the pharmaceutically-acceptable salt thereof, the tautomer thereof, the pharmaceutically-acceptable salt of the tautomer, the stereoisomer thereof, or the mixture thereof, wherein R³ is selected from —CF₃ or —OCF₃.

52. The compound of any one of embodiments 1-47 or the pharmaceutically-acceptable salt thereof, the tautomer thereof, the pharmaceutically-acceptable salt of the tautomer, the stereoisomer thereof, or the mixture thereof, wherein R³ is R^{b} and R^{b} is a phenyl substituted by 0, 1, 2 or 3 substituents selected from halo, C₁₋₄alk, C₁₋₃haloalk, —OC₁₋₄alk, —OH, —NH₂, —OC₁₋₄alk, —OC₁₋₄haloalk, —NH₁₋₄alk, and —N(C₁₋₄alk)C₁₋₄alk.

53. The compound of any one of embodiments 1-52 or the pharmaceutically-acceptable salt thereof, the tautomer thereof, the pharmaceutically-acceptable salt of the tautomer, the stereoisomer thereof, or the mixture thereof, wherein R⁴ is H.

54. The compound of any one of embodiments 1-52 or the pharmaceutically-acceptable salt thereof, the tautomer thereof, the pharmaceutically-acceptable salt of the tautomer, the stereoisomer thereof, or the mixture thereof, wherein R⁴ is selected from F, Cl, C₁₋₆alk, —OC₁₋₆alk, —OC₁₋₃haloalk, or —C₁₋₃haloalk.

55. The compound of embodiment 54 or the pharmaceutically-acceptable salt thereof, the tautomer thereof, the pharmaceutically-acceptable salt of the tautomer, the stereoisomer thereof, or the mixture thereof, wherein R⁴ is selected from —F, —Cl, or —OCF₃.

56. The compound of embodiment 55 or the pharmaceutically-acceptable salt thereof, the tautomer thereof, the pharmaceutically-acceptable salt of the tautomer, the stereoisomer thereof, or the mixture thereof, wherein R⁴ is —F.

57. The compound of any one of embodiments 1-47 or the pharmaceutically-acceptable salt thereof, the tautomer thereof, the pharmaceutically-acceptable salt of the tautomer, the stereoisomer thereof, or the mixture thereof, wherein —R³ is —OCF₃ and R⁴ is —F.

58. The compound of any one of embodiments 1-47 or the pharmaceutically-acceptable salt thereof, the tautomer thereof, the pharmaceutically-acceptable salt of the tautomer, the stereoisomer thereof, or the mixture thereof, wherein R³ and R⁴ together form a four-atom unsaturated bridge containing 0 or 1 N atoms, wherein the bridge is substituted by 0, 1 or 2 R⁵ substituents.

59. The compound of any one of embodiments 1-58 or the pharmaceutically-acceptable salt thereof, the tautomer thereof, the pharmaceutically-acceptable salt of the tautomer, the stereoisomer thereof, or the mixture thereof, wherein each instance of R⁵ is H.

60. The compound of any one of embodiments 1-58 or the pharmaceutically-acceptable salt thereof, the tautomer thereof, the pharmaceutically-acceptable salt of the tautomer, the stereoisomer thereof, or the mixture thereof, wherein in at least one instance, R⁵ is selected from C₁₋₈alk, halo, or —OR^{a}.

61. The compound of any one of embodiments 1-58 or the pharmaceutically-acceptable salt thereof, the tautomer thereof, the pharmaceutically-acceptable salt of the tautomer, the stereoisomer thereof, or the mixture thereof, wherein in at least one instance, R⁵ is selected from —CH₃, —Cl, —F, or —OMe.

62. The compound of any one of embodiments 1-61 or the pharmaceutically-acceptable salt thereof, the tautomer thereof, the pharmaceutically-acceptable salt of the tautomer, the stereoisomer thereof, or the mixture thereof, wherein each instance of R⁶ is H.

63. The compound of any one of embodiments 1-61 or the pharmaceutically-acceptable salt thereof, the tautomer thereof, the pharmaceutically-acceptable salt of the tautomer, the stereoisomer thereof, or the mixture thereof, wherein at least one instance of R⁶ is halo or C₁₋₆alk.

64. The compound of any one of embodiments 1-61 or the pharmaceutically-acceptable salt thereof, the tautomer thereof, the pharmaceutically-acceptable salt of the tautomer, the stereoisomer thereof, or the mixture thereof, wherein at least one instance of R⁶ is —F, —Cl, or —CH₃.

65. The compound of any one of embodiments 1-64 or the pharmaceutically-acceptable salt thereof, the tautomer thereof, the pharmaceutically-acceptable salt of the tautomer, the stereoisomer thereof, or the mixture thereof, wherein R¹ is the saturated, partially-saturated or unsaturated 3-, 4-, 5-, 6- or 7-membered monocyclic or 7-, 8-, 9-, 10- or 11-membered bicyclic ring and the monocyclic or bicyclic ring is substituted by 0, 1, 2, or 3 substituents, wherein the substituents are selected from F, Cl, Br, I, oxo, cyano, —CH₃, —CH₂CH₃, —CH₂CH₂CH₃, —C(H)(CH₃)₂, —CH₂C(H)(CH₃)₂, —CH₂C(H)═CF₁₂, —CH₂CO₂H, —CH₂CF₃, —C(OH)(CH₃)₂, —SO₂N(H)CH₃, —N(H)SO₂CH₃, —OCH₃, —OCF₃, —OH, —OCH₂CO₂H, —CH₂OH, —CH₂CH₂OH, —CH₂C(H)(CH₃)OH, —CO₂H, —CO₂CH₃, —CO₂CH₂CH₃, —CO₂C(CH₃)₃, —CO₂NH₂, —CO₂N(H)CH₃, —SO₂CH₃, —OC(═O)CH₃, —NH₂, —NHC(═O)CH₃, —N(CH₃)₂, —N(H)CH₂CH₃, —CF₃, —CHF₂, —CH₂C(H)(CF₃)OH, —CH₂C(CH₃)₂OH, —CH₂-phenyl, —C(═O)-phenyl, tetrazolyl, oxadiazolonyl, pyridyl, oxetanyl,

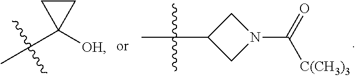 [SMILES: *C1(O)CC1 ; "or" ; *C1CN(C(=O)C(C)(C)C)C1 ; "."]

66. The compound of any one of embodiments 1-64 or the pharmaceutically-acceptable salt thereof, the tautomer thereof, the pharmaceutically-acceptable salt of the tautomer, the stereoisomer thereof, or the mixture thereof, wherein R¹ is a phenyl, pyridyl, pyridinonyl, piperidinonyl, pyridazinonyl, pyrazinonyl, pyridazinyl, pyrimidinyl, pyrazinyl, tetradyrofuranyl, tetrahydropyranyl, thiazolyl, isothiazolyl, furanyl, thiophenyl, pyrazolyl, isoxazolyl, triazolyl, oxazolyl, imidazolyl, pyrrolidinonyl, piperidinyl, cyclohexyl, cyclohexanonyl, quinolinyl, isoquinolinyl, naphthyridinyl, pyrrolopyridinyl, pyrrolopyrimidinyl, benzothiophenyl, pyrazolopyrimidinyl, triazolopyrimidinyl, indazolyl, tetrahydroindazolyl, tetrahydrocyclopentapyrazolyl, dihydropyrazolooxazinyl, indolinonyl, isoindolinonyl, benzooxazolonyl, oxazolopyridinonyl, benzoimidazolonyl, isoindolindionyl, tetrahydroquinolinyl, dihydroquinolinonyl, benzooxazinonyl, dihydrobenzooxazinonyl, dihydroindenonyl, benzothiazolyl, benzimidazolyl, imidazopyridinyl, tetrazolopyridinyl, quinolinonyl, quinoxalinyl, indolyl, or quinoxalindionyl substituted by 0, 1, 2, or 3 substituents.

67. The compound of any one of embodiments 1-64 or the pharmaceutically-acceptable salt thereof, the tautomer thereof, the pharmaceutically-acceptable salt of the tautomer, the stereoisomer thereof, or the mixture thereof, wherein R¹ is a phenyl, pyridyl, pyridinonyl, pyrazinonyl, pyridazinyl, pyrimidinyl, tetrahydropyranyl, thiazolyl, isothiazolyl, imidazolyl, piperidinyl, quinolinyl, isoquinolinyl, indazolyl, indolinonyl, isoindolinonyl, benzooxazolonyl, dihydroquinolinonyl, imidazopyridinyl, quinolinonyl, indolyl substituted by 0, 1, 2, or 3 substituents.

68. The compound of embodiment 67 or the pharmaceutically-acceptable salt thereof, the tautomer thereof, the pharmaceutically-acceptable salt of the tautomer, the stereoisomer thereof, or the mixture thereof, wherein R¹ is a phenyl substituted by 0, or 1 substituent.

69. The compound of embodiment 67 or the pharmaceutically-acceptable salt thereof, the tautomer thereof, the pharmaceutically-acceptable salt of the tautomer, the stereoisomer thereof, or the mixture thereof, wherein R¹ is a pyridinonyl substituted by 0, or 1 substituent.

70. The compound of embodiment 67 or the pharmaceutically-acceptable salt thereof, the tautomer thereof, the pharmaceutically-acceptable salt of the tautomer, the stereoisomer thereof, or the mixture thereof, wherein R¹ is a pyridyl substituted by 0, or 1 substituent.

71. The compound of embodiment 67 or the pharmaceutically-acceptable salt thereof, the tautomer thereof, the pharmaceutically-acceptable salt of the tautomer, the stereoisomer thereof, or the mixture thereof, wherein R¹ is a benzooxazolonyl substituted by 0, or 1 substituent.

72. The compound of embodiment 67 or the pharmaceutically-acceptable salt thereof, the tautomer thereof, the pharmaceutically-acceptable salt of the tautomer, the stereoisomer thereof, or the mixture thereof, wherein R¹ is a quinolinyl substituted by 0, or 1 substituent.

73. The compound of any one of embodiments 1-64 or the pharmaceutically-acceptable salt thereof, the tautomer thereof, the pharmaceutically-acceptable salt of the tautomer, the stereoisomer thereof, or the mixture thereof, wherein R¹ is a group of formula

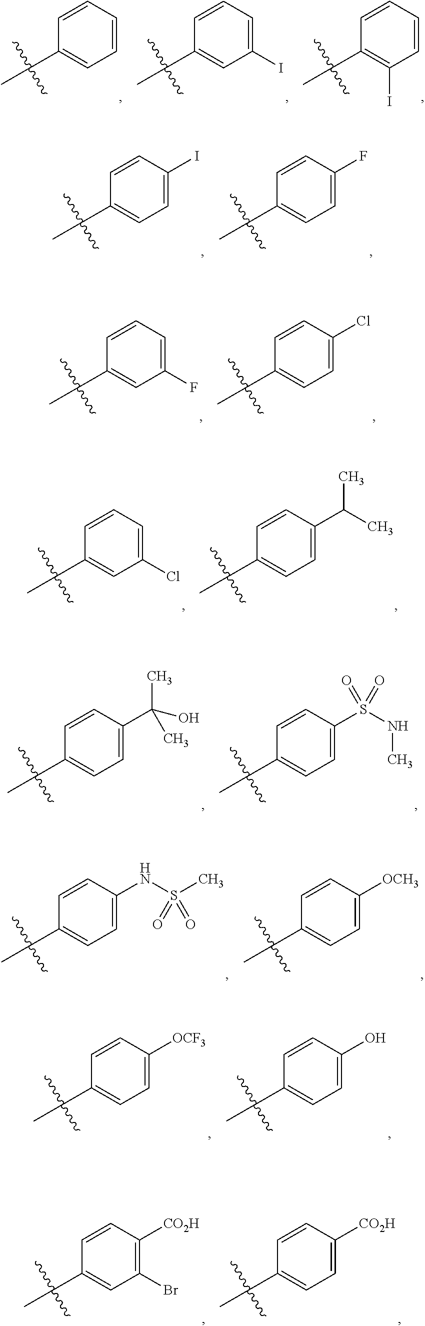

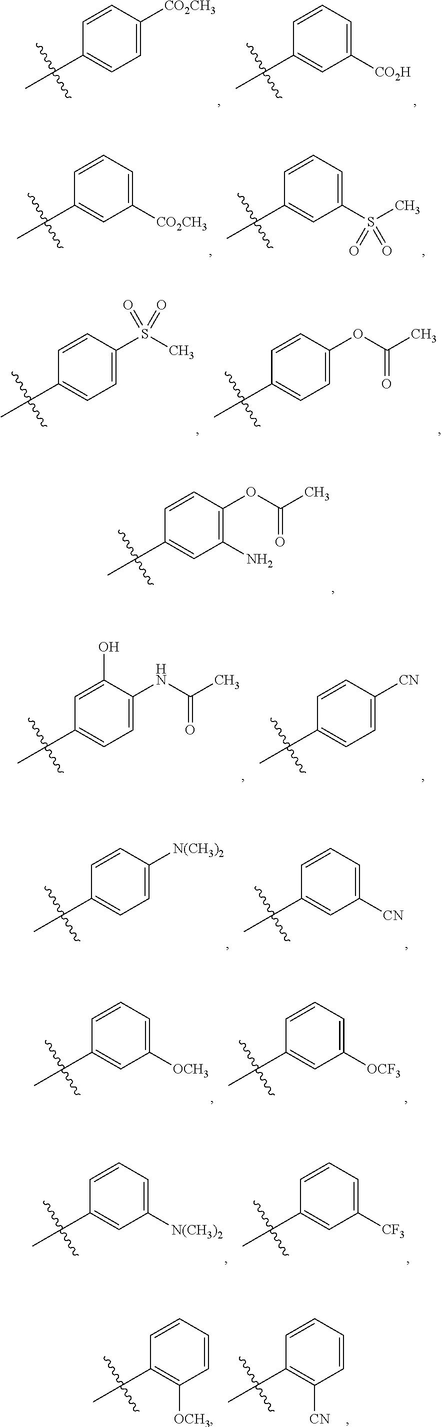

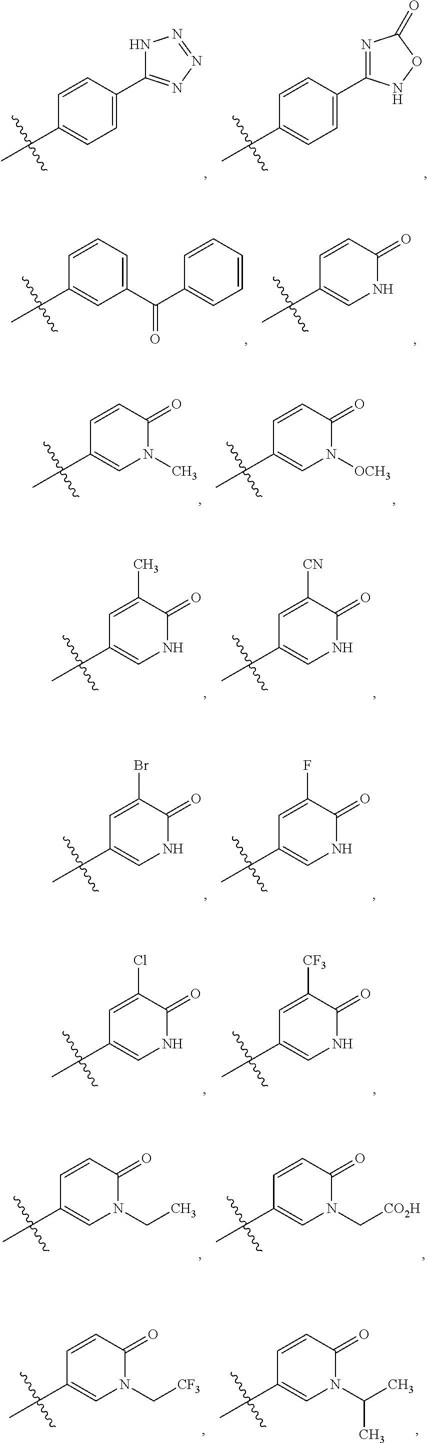

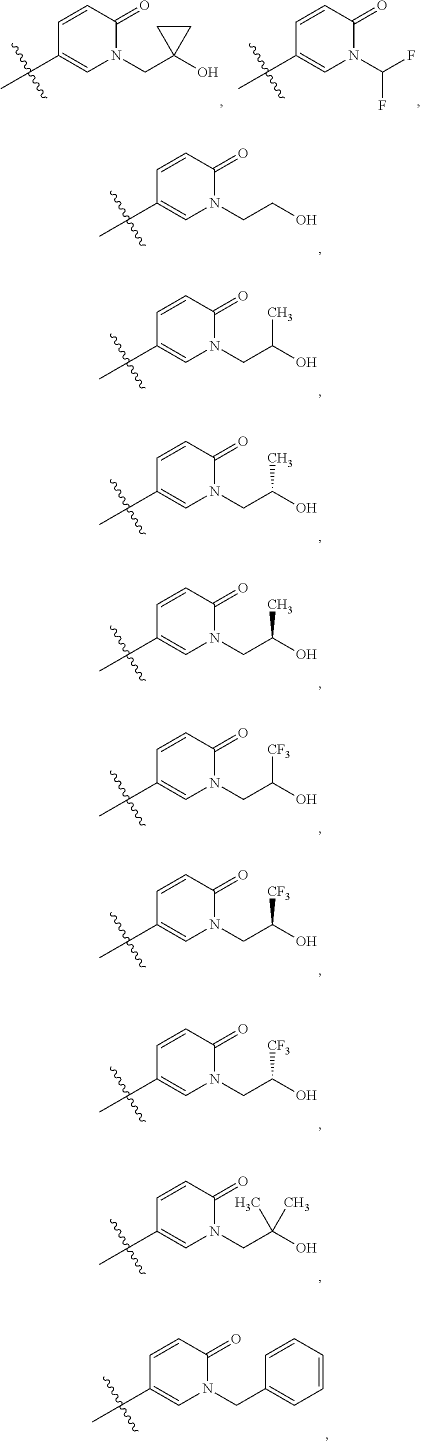

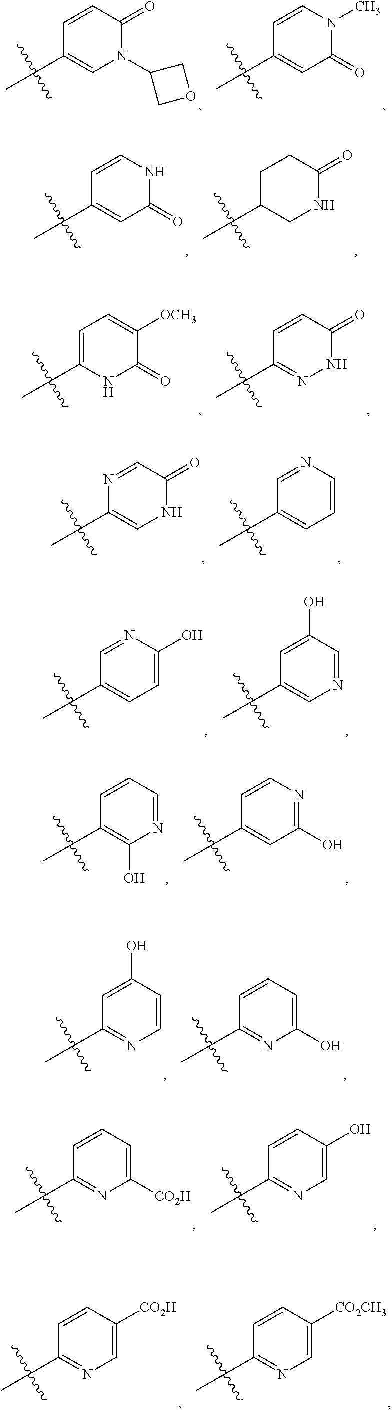

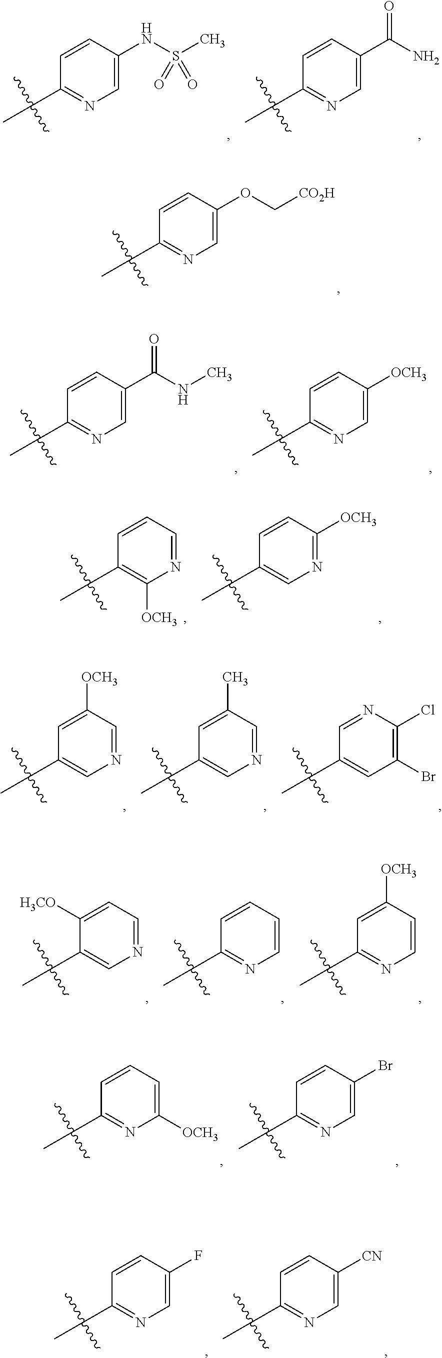

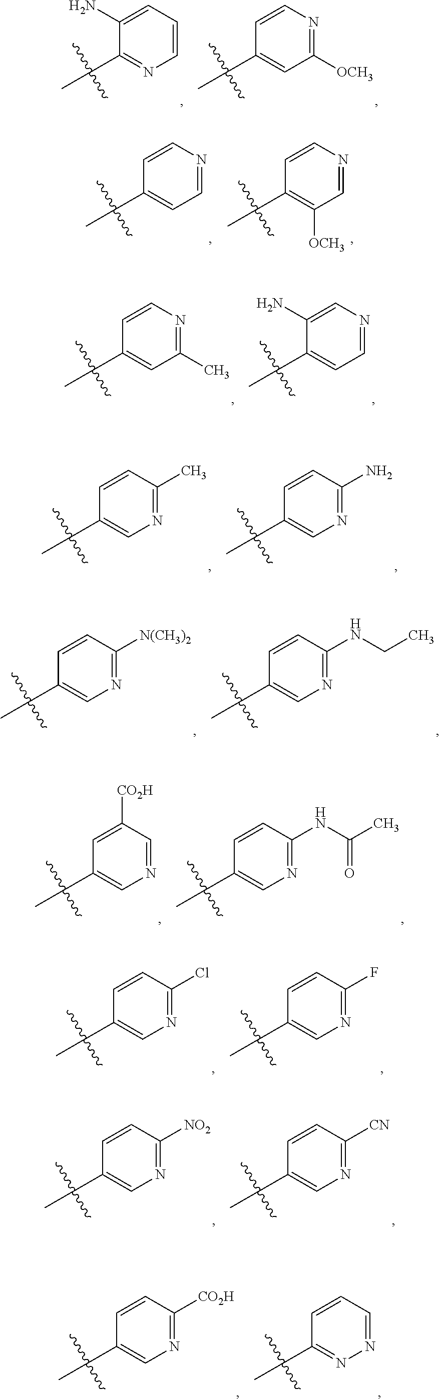

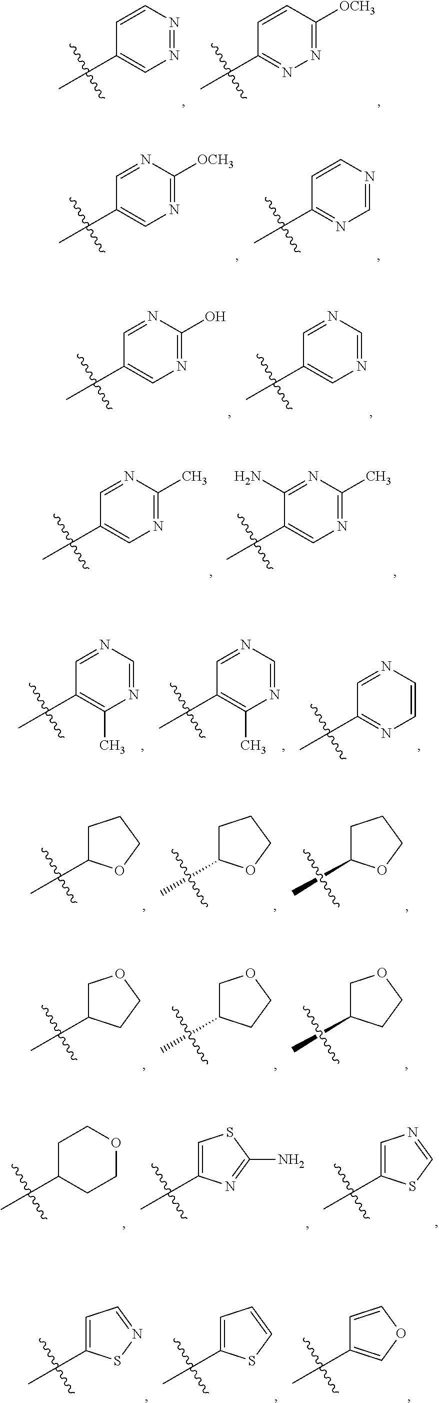

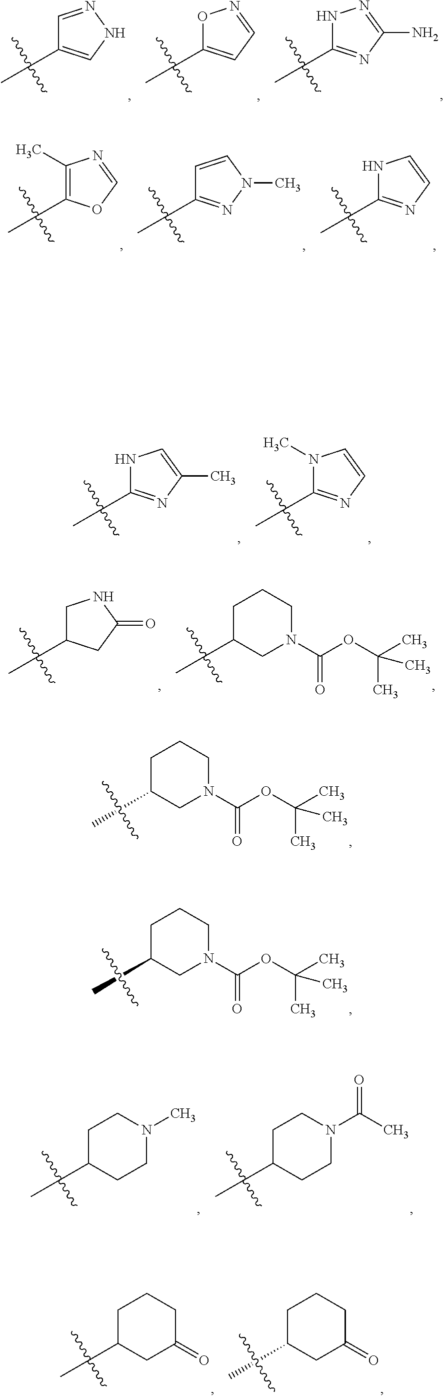

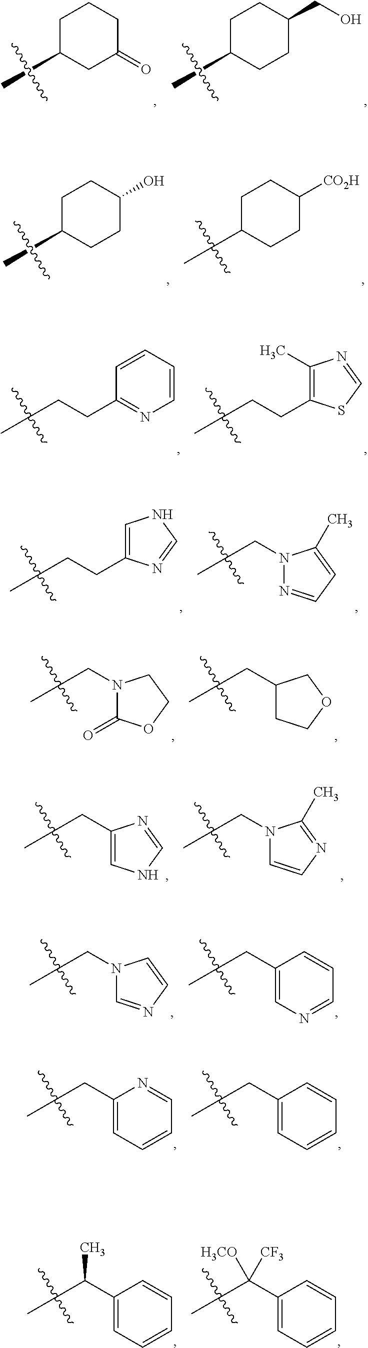

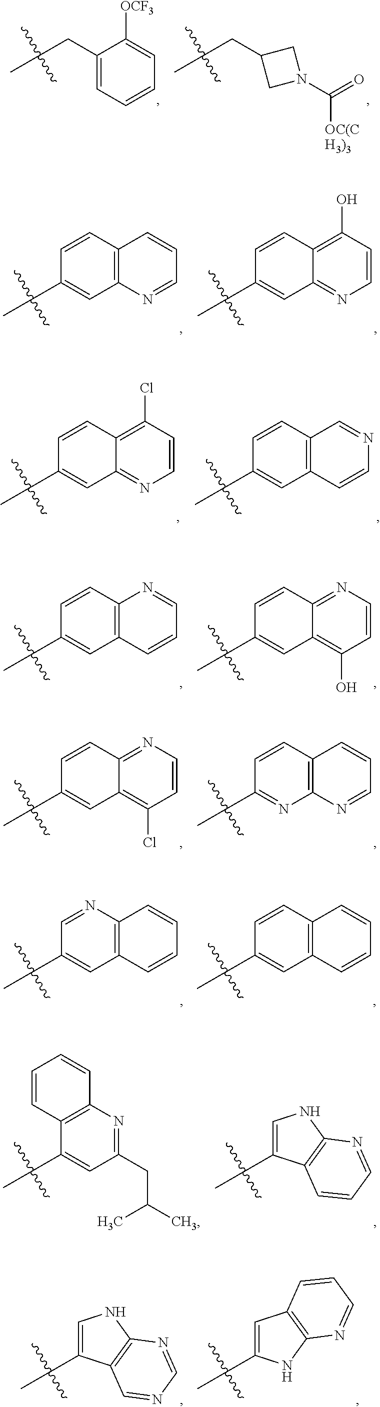

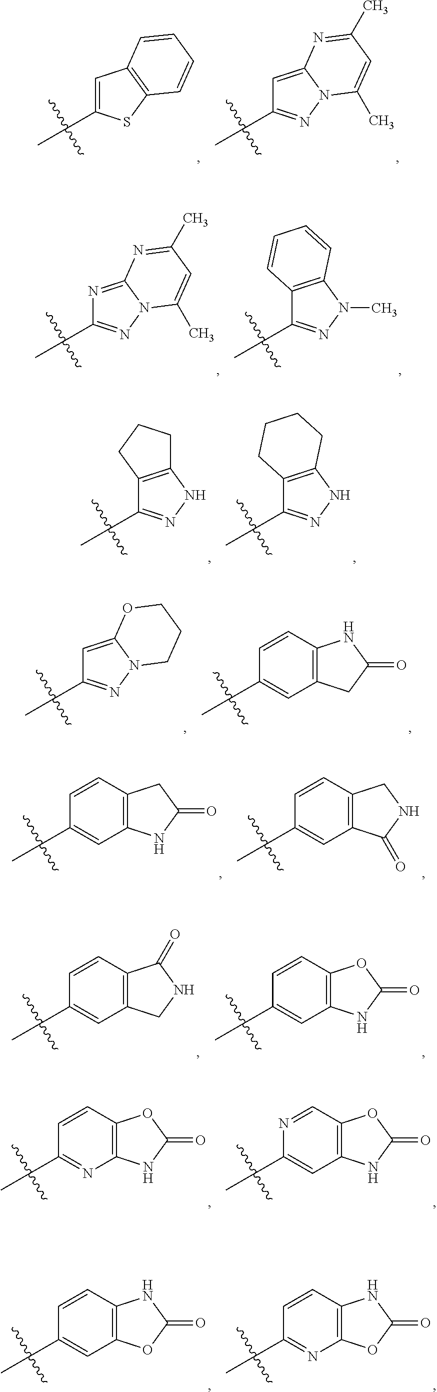

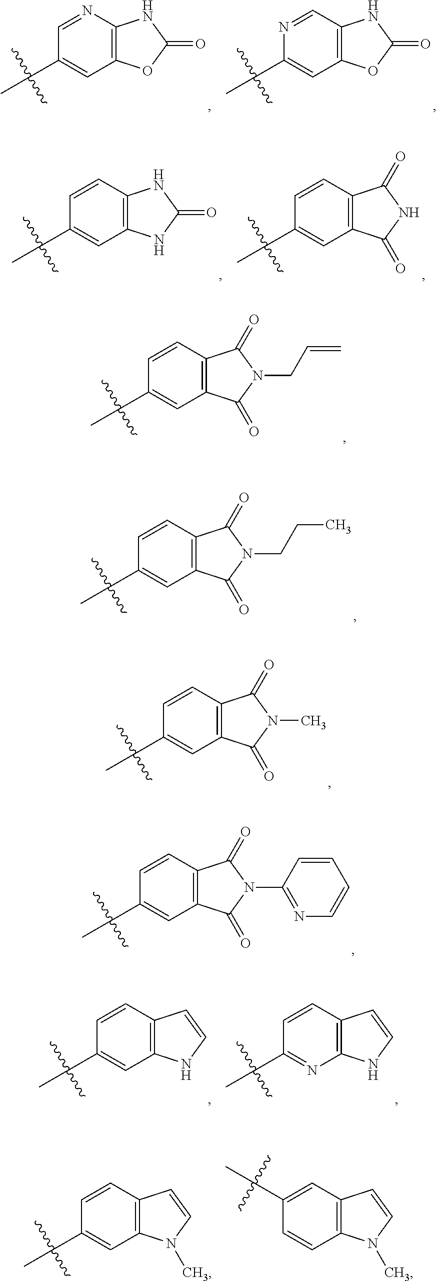

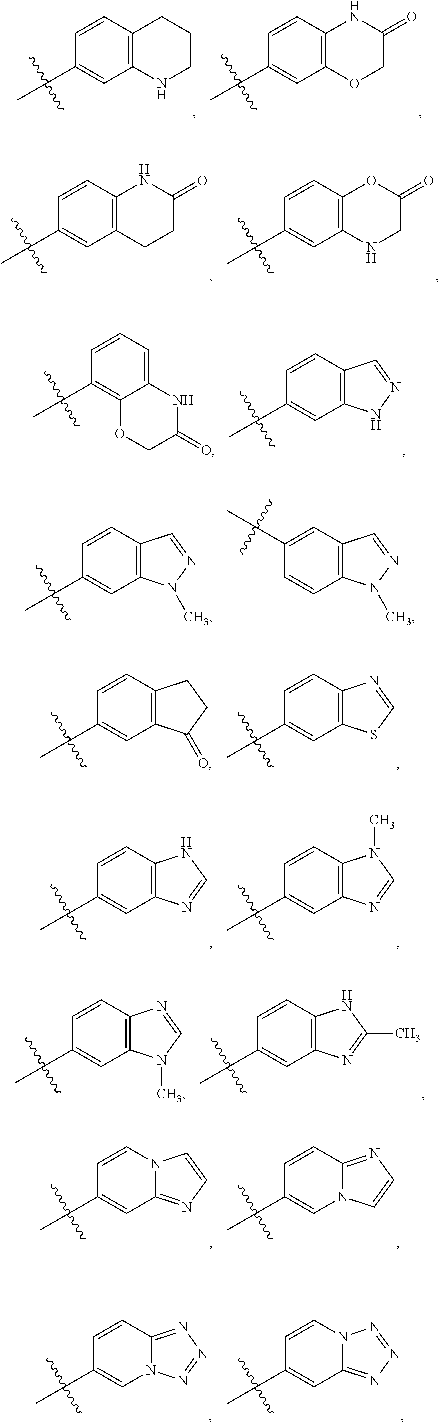

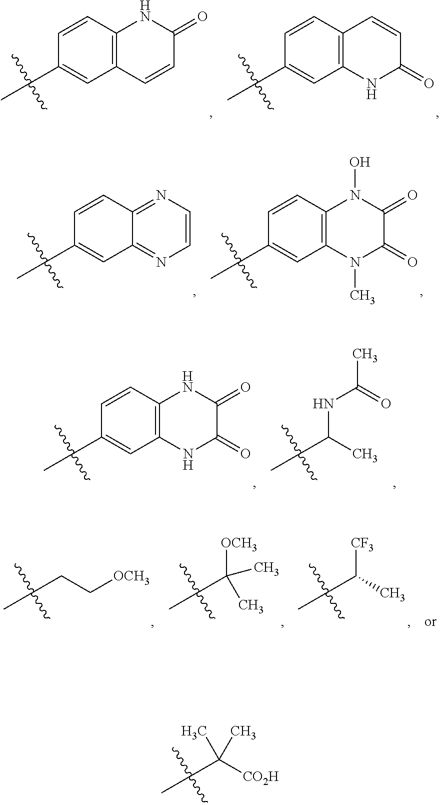

and the symbol [symbol] , when drawn across a bond, indicates the point of attachment to the rest of the molecule.

74. The compound of any one of embodiments 1-64 or the pharmaceutically-acceptable salt thereof, the tautomer thereof, the pharmaceutically-acceptable salt of the tautomer, the stereoisomer thereof, or the mixture thereof, wherein R¹ is a group of formula

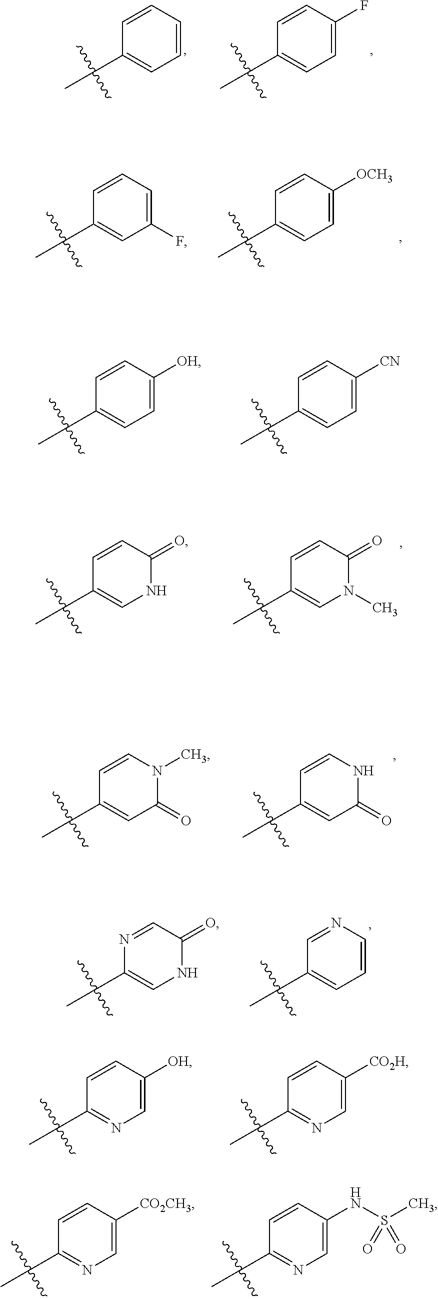

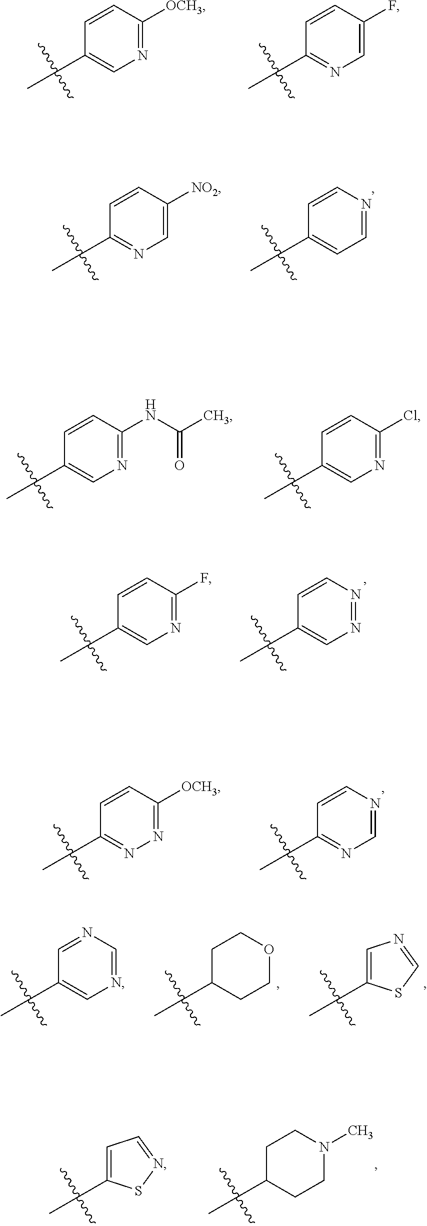

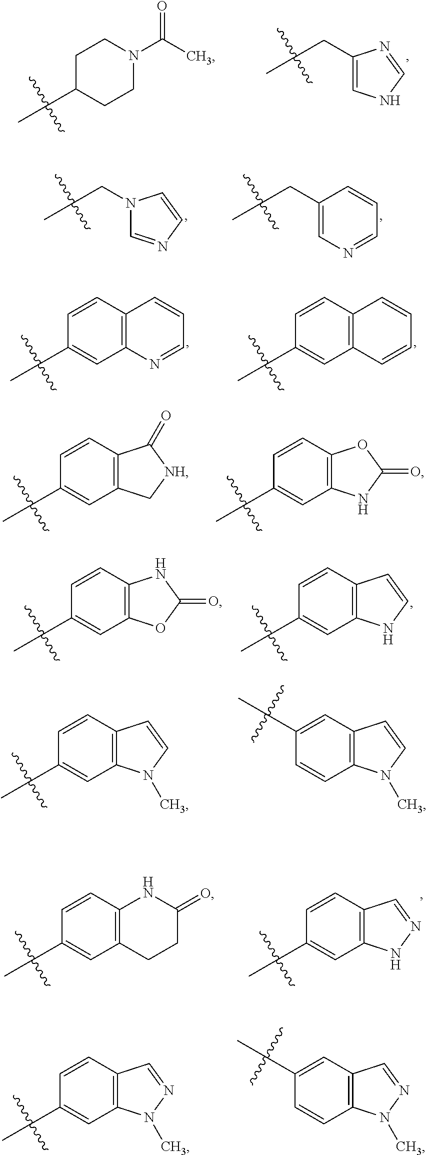

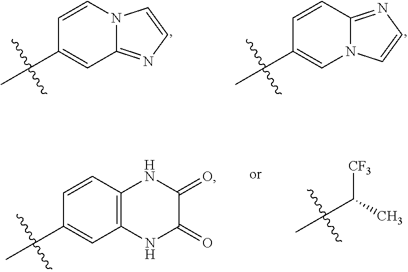

the symbol [symbol] , when drawn across a bond, indicates the point of attachment to the rest of the molecule.

75. The compound of any one of embodiments 1-64 or the pharmaceutically-acceptable salt hereof, the tautomer thereof, the pharmaceutically-acceptable salt of the tautomer, the stereoisomer thereof, or the mixture thereof, wherein R¹ is a group of formula

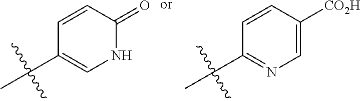 [SMILES: *c1ccc(=O)[nH]c1 ; "or" ; *c1ccc(C(=O)O)cn1]

and the symbol [symbol] , when drawn across a bond, indicates the point of attachment to the rest of the molecule.

76. The compound of any one of embodiments 1-64 or the pharmaceutically-acceptable salt thereof, the tautomer thereof, the pharmaceutically-acceptable salt of the tautomer, the stereoisomer thereof, or the mixture thereof, wherein R¹ is

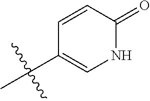 [SMILES: *c1ccc(=O)[nH]c1]

and the symbol [symbol] , when drawn across a bond, indicates the point of attachment to the rest of the molecule.

77. The compound of any one of embodiments 1-64 or the pharmaceutically-acceptable salt thereof, the tautomer thereof, the pharmaceutically-acceptable salt of the tautomer, the stereoisomer thereof, or the mixture thereof, wherein R¹ is

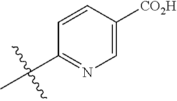 [SMILES: *c1ccc(C(=O)O)cn1]

and the symbol [symbol] , when drawn across a bond, indicates the point of attachment to the rest of the molecule.

78. The compound of embodiment 1, wherein the compound is
(S)—N-(4-(4-(trifluoromethyl)phenyl)-3,4-dihydro-2H-pyrano[3,2-b]pyridin-4-yl)benzamide;
(S)-4-fluoro-N-(4-(4-(trifluoromethyl)phenyl)-3,4-dihydro-2H-pyrano[3,2-b]pyridin-4-yl)benzamide;
(S)—N-(4-(3,4-dichlorophenyl)-3,4-dihydro-2H-pyrano[3,2-b]pyridin-4-yl)benzamide;
(S)-4-hydroxy-N-(4-(4-(trifluoromethyl)phenyl)-3,4-dihydro-2H-pyrano[3,2-b]pyridin-4-yl)benzamide;
(S)-4-methoxy-N-(4-(4-(trifluoromethyl)phenyl)-3,4-dihydro-2H-pyrano[3,2-b]pyridin-4-yl)benzamide;
4-fluoro-N-(4-(3-fluoro-4-(trifluoromethoxy)phenyl)-3,4-dihydro-2H-pyrano[3,2-b]pyridin-4-yl)benzamide;
N-(4-(3-fluoro-4-(trifluoromethoxy)phenyl)-3,4-dihydro-2H-pyrano[3,2-b]pyridin-4-yl)benzamide;
4-fluoro-N-(4-(4-(trifluoromethoxy)phenyl)-3,4-dihydro-2H-pyrano[3,2-b]pyridin-4-yl)benzamide;
N-(4-(4-(trifluoromethoxy)phenyl)-3,4-dihydro-2H-pyrano[3,2-b]pyridin-4-yl)benzamide;
N-(4-(3,4-dichlorophenyl)-3,4-dihydro-2H-pyrano[3,2-b]pyridin-4-yl)-4-fluorobenzamide;
4-fluoro-N-(4-phenyl-3,4-dihydro-2H-pyrano[3,2-b]pyridin-4-yl)benzamide;
N-(4-phenyl-3,4-dihydro-2H-pyrano[3,2-b]pyridin-4-yl)benzamide;
N-(4-(6-(trifluoromethyl)pyridin-3-yl)-3,4-dihydro-2H-pyrano[3,2-b]pyridin-4-yl)benzamide;
(S)—N-(4-(4-chlorophenyl)-3,4-dihydro-2H-pyrano[3,2-b]pyridin-4-yl)benzamide;
(S)—N-(4-(4-chloro-3-fluorophenyl)-3,4-dihydro-2H-pyrano[3,2-b]pyridin-4-yl)benzamide;
(S)—N-(4-(4-fluorophenyl)-3,4-dihydro-2H-pyrano[3,2-b]pyridin-4-yl)benzamide;
(S)—N-(4-(3-(trifluoromethoxy)phenyl)-3,4-dihydro-2H-pyrano[3,2-b]pyridin-4-yl)benzamide;
(S)—N-(4-(p-tolyl)-3,4-dihydro-2H-pyrano[3,2-b]pyridin-4-yl)benzamide;
(S)—N-(4-(3-fluoro-4-(trifluoromethyl)phenyl)-3,4-dihydro-2H-pyrano[3,2-b]pyridin-4-yl)benzamide;
(S)-2-oxo-N-(4-(4-(trifluoromethyl)phenyl)-3,4-dihydro-2H-pyrano[3,2-b]pyridin-4-yl)-2,3-dihydrobenzo[d]oxazole-6-carboxamide;
(S)—N-(4-(3-fluoro-4-(trifluoromethoxy)phenyl)-3,4-dihydro-2H-pyrano[3,2-b]pyridin-4-yl)-2-oxo-2,3-dihydrobenzo[d]oxazole-6-carboxamide;
2-oxo-N-(4-(4-(trifluoromethoxy)phenyl)-3,4-dihydro-2H-pyrano[3,2-b]pyridin-4-yl)-2,3-dihydrobenzo[d]oxazole-6-carboxamide;
2-oxo-N-(4-(4-(trifluoromethoxy)phenyl)-3,4-dihydro-2H-pyrano[3,2-b]pyridin-4-yl)-2,3-dihydrobenzo[d]oxazole-5-carboxamide;
N-(4-(3,4-dichlorophenyl)-3,4-dihydro-2H-pyrano[3,2-b]pyridin-4-yl)-2-oxo-2,3-dihydrobenzo[d]oxazole-6-carboxamide;
N-(4-(3,4-dichlorophenyl)-3,4-dihydro-2H-pyrano[3,2-b]pyridin-4-yl)-2-oxo-2,3-dihydrobenzo[d]oxazole-5-carboxamide;
2-oxo-N-(4-phenyl-3,4-dihydro-2H-pyrano[3,2-b]pyridin-4-yl)-2,3-dihydrobenzo[d]oxazole-6-carboxamide;
2-oxo-N-(4-(6-(trifluoromethyl)pyridin-3-yl)-3,4-dihydro-2H-pyrano[3,2-b]pyridin-4-yl)-2,3-dihydrobenzo[d]oxazole-5-carboxamide;
(S)—N-(4-(4-(trifluoromethyl)phenyl)chroman-4-yl)benzamide;
N-(8-(4-(trifluoromethyl)phenyl)-5,6,7,8-tetrahydroquinolin-8-yl)benzamide;
methyl 6-((4-(3-fluoro-4-(trifluoromethoxy)phenyl)-3,4-dihydro-2H-pyrano[3,2-b]pyridin-4-yl)carbamoyl)nicotinate;
6-oxo-N-(8-(4-(trifluoromethyl)phenyl)-5,6,7,8-tetrahydroquinolin-8-yl)-1,6-dihydropyridine-3-carboxamide;
(S)-6-methoxy-N-(4-(4-(trifluoromethyl)phenyl)-3,4-dihydro-2H-pyrano[3,2-b]pyridin-4-yl)nicotinamide;
(S)-2-oxo-N-(4-(4-(trifluoromethyl)phenyl)-3,4-dihydro-2H-pyrano[3,2-b]pyridin-4-yl)-1,2,3,4-tetrahydroquinoline-6-carboxamide;
(S)—N-(4-(4-(trifluoromethyl)phenyl)-3,4-dihydro-2H-pyrano[3,2-b]pyridin-4-yl)-1H-indazole-6-carboxamide;
(S)—N-(4-(4-(trifluoromethyl)phenyl)-3,4-dihydro-2H-pyrano[3,2-b]pyridin-4-yl)pyrimidine-4-carboxamide;
(S)—N-(4-(4-(trifluoromethyl)phenyl)-3,4-dihydro-2H-pyrano[3,2-b]pyridin-4-yl)-1H-indole-6-carboxamide;
(S)—N-(4-(4-(trifluoromethyl)phenyl)-3,4-dihydro-2H-pyrano[3,2-b]pyridin-4-yl)pyridazine-4-carboxamide;
(S)-1-methyl-N-(4-(4-(trifluoromethyl)phenyl)-3,4-dihydro-2H-pyrano[3,2-b]pyridin-4-yl)-1H-indazole-6-carboxamide;
(S)—N-(4-(4-(trifluoromethyl)phenyl)-3,4-dihydro-2H-pyrano[3,2-b]pyridin-4-yl)thiazole-5-carboxamide;
(S)-5-hydroxy-N-(4-(4-(trifluoromethyl)phenyl)-3,4-dihydro-2H-pyrano[3,2-b]pyridin-4-yl)picolinamide;
(S)—N-(4-(4-(trifluoromethyl)phenyl)-3,4-dihydro-2H-pyrano[3,2-b]pyridin-4-yl)isothiazole-5-carboxamide;
(S)-1-methyl-N-(4-(4-(trifluoromethyl)phenyl)-3,4-dihydro-2H-pyrano[3,2-b]pyridin-4-yl)-1H-indole-5-carboxamide;
(S)-5-acetamido-N-(4-(4-(trifluoromethyl)phenyl)-3,4-dihydro-2H-pyrano[3,2-b]pyridin-4-yl)picolinamide;
(S)-1-methyl-N-(4-(4-(trifluoromethyl)phenyl)-3,4-dihydro-2H-pyrano[3,2-b]pyridin-4-yl)-1H-indazole-5-carboxamide;
(S)-5-oxo-N-(4-(4-(trifluoromethyl)phenyl)-3,4-dihydro-2H-pyrano[3,2-b]pyridin-4-yl)-4,5-dihydropyrazine-2-carboxamide;
(S)—N-(4-(4-(trifluoromethyl)phenyl)-3,4-dihydro-2H-pyrano[3,2-b]pyridin-4-yl)isonicotinamide;
(S)-6-methoxy-N-(4-(4-(trifluoromethyl)phenyl)-3,4-dihydro-2H-pyrano[3,2-b]pyridin-4-yl)pyridazine-3-carboxamide;
(S)—N-(4-(4-(trifluoromethyl)phenyl)-3,4-dihydro-2H-pyrano[3,2-b]pyridin-4-yl)nicotinamide;
(S)—N-(4-(4-(trifluoromethyl)phenyl)-3,4-dihydro-2H-pyrano[3,2-b]pyridin-4-yl)imidazo[1,2-a]pyridine-7-carboxamide;
(S)—N-(4-(3-fluoro-4-(trifluoromethoxy)phenyl)-3,4-dihydro-2H-pyrano[3,2-b]pyridin-4-yl)-5-oxo-4,5-dihydropyrazine-2-carboxamide;
(S)—N-(4-(3-fluoro-4-(trifluoromethoxy)phenyl)-3,4-dihydro-2H-pyrano[3,2-b]pyridin-4-yl)-5-hydroxypicolinamide;
(S)—N-(4-(3-fluoro-4-(trifluoromethoxy)phenyl)-3,4-dihydro-2H-pyrano[3,2-b]pyridin-4-yl)imidazo[1,2-a]pyridine-6-carboxamide;
(S)—N-(4-(3-fluoro-4-(trifluoromethoxy)phenyl)-3,4-dihydro-2H-pyrano[3,2-b]pyridin-4-yl)-1-methyl-1H-indazole-5-carboxamide;
(S)—N-(4-(3-fluoro-4-(trifluoromethoxy)phenyl)-3,4-dihydro-2H-pyrano[3,2-b]pyridin-4-yl)-2-oxo-1,2,3,4-tetrahydroquinoline-6-carboxamide;
(S)-5-fluoro-N-(4-(3-fluoro-4-(trifluoromethoxy)phenyl)-3,4-dihydro-2H-pyrano[3,2-b]pyridin-4-yl)picolinamide;
(S)—N-(4-(4-(trifluoromethyl)phenyl)-3,4-dihydro-2H-pyrano[3,2-b]pyridin-4-yl)quinoline-7-carboxamide;
(S)—N-(4-(3-fluoro-4-(trifluoromethoxy)phenyl)-3,4-dihydro-2H-pyrano[3,2-b]pyridin-4-yl)-2-oxo-2,3-dihydrobenzo[d]oxazole-5-carboxamide;
(S)-6-oxo-N-(4-(4-(trifluoromethoxy)phenyl)-3,4-dihydro-2H-pyrano[3,2-b]pyridin-4-yl)-1,6-dihydropyridine-3-carboxamide;
(S)—N-(4-(3-fluoro-4-(trifluoromethoxy)phenyl)-3,4-dihydro-2H-pyrano[3,2-b]pyridin-4-yl)-6-oxo-1,6-dihydropyridine-3-carboxamide;
(S)-6-oxo-N-(4-(4-(trifluoromethyl)phenyl)-3,4-dihydro-2H-pyrano[3,2-b]pyridin-4-yl)-1,6-dihydropyridine-3-carboxamide;
(S)-2-oxo-N-(4-(4-(trifluoromethyl)phenyl)-3,4-dihydro-2H-pyrano[3,2-b]pyridin-4-yl)-2,3-dihydrobenzo[d]oxazole-5-carboxamide;
(S)—N-(4-(3-fluoro-4-(trifluoromethoxy)phenyl)-3,4-dihydro-2H-pyrano[3,2-b]pyridin-4-yl)-5-nitropicolinamide;
(S)—N5-(4-(3-fluoro-4-(trifluoromethoxy)phenyl)-3,4-dihydro-2H-pyrano[3,2-b]pyridin-4-yl)-N2-methylpyridine-2,5-dicarboxamide;
(S)—N-(4-(3-fluoro-4-(trifluoromethoxy)phenyl)-3,4-dihydro-2H-pyrano[3,2-b]pyridin-4-yl)-2,3-dioxo-1,2,3,4-tetrahydroquinoxaline-6-carboxamide;
(S)—N-(4-(3-fluoro-4-(trifluoromethoxy)phenyl)-3,4-dihydro-2H-pyrano[3,2-b]pyridin-4-yl)-5-(methylsulfonamido)picolinamide;
(S)-1-(4-(3-fluoro-4-(trifluoromethoxy)phenyl)-3,4-dihydro-2H-pyrano[3,2-b]pyridin-4-yl)-3-(6-fluoropyridin-3-yl)urea;
1-((S)-4-(4-(trifluoromethyl)phenyl)-3,4-dihydro-2H-pyrano[3,2-b]pyridin-4-yl)-3-((S)-1,1,1-trifluoropropan-2-yl)urea;
(S)-1-(4-cyanophenyl)-3-(4-(3-fluoro-4-(trifluoromethoxy)phenyl)-3,4-dihydro-2H-pyrano[3,2-b]pyridin-4-yl)urea;
(S)-1-(6-chloropyridin-3-yl)-3-(4-(3-fluoro-4-(trifluoromethoxy)phenyl)-3,4-dihydro-2H-pyrano[3,2-b]pyridin-4-yl)urea;
1-(pyridin-3-yl)-3-(4-(4-(trifluoromethyl)phenyl)chroman-4-yl)urea;
1-(pyridin-3-yl)-3-(8-(4-(trifluoromethyl)phenyl)-5,6,7,8-tetrahydroquinolin-8-yl)urea;
(S)-1-(3-fluorophenyl)-3-(4-(4-(trifluoromethyl)phenyl)-3,4-dihydro-2H-pyrano[3,2-b]pyridin-4-yl)urea;
(S)-1-(pyridin-3-yl)-3-(4-(4-(trifluoromethyl)phenyl)-3,4-dihydro-2H-pyrano[3,2-b]pyridin-4-yl)urea;
(S)-1-(4-fluorophenyl)-3-(4-(4-(trifluoromethyl)phenyl)-3,4-dihydro-2H-pyrano[3,2-b]pyridin-4-yl)urea;
(S)-6-((4-(3-fluoro-4-(trifluoromethoxy)phenyl)-3,4-dihydro-2H-pyrano[3,2-b]pyridin-4-yl)carbamoyl)nicotinic acid;
(S)—N-(4-(3-fluoro-4-(trifluoromethoxy)phenyl)-3,4-dihydro-2H-pyrano[3,2-b]pyridin-4-yl)benzenesulfonamide;
(S)—N-(4-(3-fluoro-4-(trifluoromethoxy)phenyl)-3,4-dihydro-2H-pyrano[3,2-b]pyridin-4-yl)pyridine-3-sulfonamide;
(S)-tert-butyl (4-(3-fluoro-4-(trifluoromethoxy)phenyl)-3,4-dihydro-2H-pyrano[3,2-b]pyridin-4-yl)carbamate;
1-methyl-6-oxo-N-(4-(4-(trifluoromethyl)phenyl)-3,4-dihydro-2H-pyrano[3,2-b]pyridin-4-yl)-1,6-dihydropyridine-3-carboxamide;
1-oxo-N-(4-(4-(trifluoromethyl)phenyl)-3,4-dihydro-2H-pyrano[3,2-b]pyridin-4-yl)isoindoline-5-carboxamide; or
(S)—N-(4-(naphthalen-2-yl)-3,4-dihydro-2H-pyrano[3,2-b]pyridin-4-yl)-6-oxo-1,6-dihydropyridine-3-carboxamide; or
   the pharmaceutically-acceptable salt thereof, the tautomer thereof, the pharmaceutically-acceptable salt of the tautomer, the stereoisomer thereof, or the mixture thereof.

79. The compound of embodiment 1, wherein the compound is
(S)—N-(4-(4-(trifluoromethyl)phenyl)-3,4-dihydro-2H-pyrano[3,2-b]pyridin-4-yl)benzamide;
(S)-4-fluoro-N-(4-(4-(trifluoromethyl)phenyl)-3,4-dihydro-2H-pyrano[3,2-b]pyridin-4-yl)benzamide;
(S)—N-(4-(3,4-dichlorophenyl)-3,4-dihydro-2H-pyrano[3,2-b]pyridin-4-yl)benzamide;
(S)-4-hydroxy-N-(4-(4-(trifluoromethyl)phenyl)-3,4-dihydro-2H-pyrano[3,2-b]pyridin-4-yl)benzamide;
(S)-4-methoxy-N-(4-(4-(trifluoromethyl)phenyl)-3,4-dihydro-2H-pyrano[3,2-b]pyridin-4-yl)benzamide;
4-fluoro-N-(4-(3-fluoro-4-(trifluoromethoxy)phenyl)-3,4-dihydro-2H-pyrano[3,2-b]pyridin-4-yl)benzamide;
N-(4-(3-fluoro-4-(trifluoromethoxy)phenyl)-3,4-dihydro-2H-pyrano[3,2-b]pyridin-4-yl)benzamide;
4-fluoro-N-(4-(4-(trifluoromethoxy)phenyl)-3,4-dihydro-2H-pyrano[3,2-b]pyridin-4-yl)benzamide;
N-(4-(4-(trifluoromethoxy)phenyl)-3,4-dihydro-2H-pyrano[3,2-b]pyridin-4-yl)benzamide;
N-(4-(3,4-dichlorophenyl)-3,4-dihydro-2H-pyrano[3,2-b]pyridin-4-yl)-4-fluorobenzamide;
4-fluoro-N-(4-phenyl-3,4-dihydro-2H-pyrano[3,2-b]pyridin-4-yl)benzamide;
N-(4-phenyl-3,4-dihydro-2H-pyrano[3,2-b]pyridin-4-yl)benzamide;
N-(4-(6-(trifluoromethyl)pyridin-3-yl)-3,4-dihydro-2H-pyrano[3,2-b]pyridin-4-yl)benzamide;
(S)—N-(4-(4-chlorophenyl)-3,4-dihydro-2H-pyrano[3,2-b]pyridin-4-yl)benzamide;
(S)—N-(4-(4-chloro-3-fluorophenyl)-3,4-dihydro-2H-pyrano[3,2-b]pyridin-4-yl)benzamide;
(S)—N-(4-(4-fluorophenyl)-3,4-dihydro-2H-pyrano[3,2-b]pyridin-4-yl)benzamide;
(S)—N-(4-(3-(trifluoromethoxy)phenyl)-3,4-dihydro-2H-pyrano[3,2-b]pyridin-4-yl)benzamide;
(S)—N-(4-(p-tolyl)-3,4-dihydro-2H-pyrano[3,2-b]pyridin-4-yl)benzamide;
(S)—N-(4-(3-fluoro-4-(trifluoromethyl)phenyl)-3,4-dihydro-2H-pyrano[3,2-b]pyridin-4-yl)benzamide;
(S)—N-(4-(3-fluoro-4-(trifluoromethoxy)phenyl)-3,4-dihydro-2H-pyrano[3,2-b]pyridin-4-yl)-2-oxo-2,3-dihydrobenzo[d]oxazole-6-carboxamide;
2-oxo-N-(4-(4-(trifluoromethoxy)phenyl)-3,4-dihydro-2H-pyrano[3,2-b]pyridin-4-yl)-2,3-dihydrobenzo[d]oxazole-6-carboxamide;
2-oxo-N-(4-(4-(trifluoromethoxy)phenyl)-3,4-dihydro-2H-pyrano[3,2-b]pyridin-4-yl)-2,3-dihydrobenzo[d]oxazole-5-carboxamide;
N-(4-(3,4-dichlorophenyl)-3,4-dihydro-2H-pyrano[3,2-b]pyridin-4-yl)-2-oxo-2,3-dihydrobenzo[d]oxazole-6-carboxamide;
N-(4-(3,4-dichlorophenyl)-3,4-dihydro-2H-pyrano[3,2-b]pyridin-4-yl)-2-oxo-2,3-dihydrobenzo[d]oxazole-5-carboxamide;
2-oxo-N-(4-phenyl-3,4-dihydro-2H-pyrano[3,2-b]pyridin-4-yl)-2,3-dihydrobenzo[d]oxazole-6-carboxamide;
2-oxo-N-(4-(6-(trifluoromethyl)pyridin-3-yl)-3,4-dihydro-2H-pyrano[3,2-b]pyridin-4-yl)-2,3-dihydrobenzo[d]oxazole-5-carboxamide;
(S)—N-(4-(4-(trifluoromethyl)phenyl)chroman-4-yl)benzamide;
N-(8-(4-(trifluoromethyl)phenyl)-5,6,7,8-tetrahydroquinolin-8-yl)benzamide;
methyl 6-((4-(3-fluoro-4-(trifluoromethoxy)phenyl)-3,4-dihydro-2H-pyrano[3,2-b]pyridin-4-yl)carbamoyl)nicotinate;
6-oxo-N-(8-(4-(trifluoromethyl)phenyl)-5,6,7,8-tetrahydroquinolin-8-yl)-1,6-dihydropyridine-3-carboxamide;
(S)-6-methoxy-N-(4-(4-(trifluoromethyl)phenyl)-3,4-dihydro-2H-pyrano[3,2-b]pyridin-4-yl)nicotinamide;
(S)-2-oxo-N-(4-(4-(trifluoromethyl)phenyl)-3,4-dihydro-2H-pyrano[3,2-b]pyridin-4-yl)-1,2,3,4-tetrahydroquinoline-6-carboxamide;
(S)—N-(4-(4-(trifluoromethyl)phenyl)-3,4-dihydro-2H-pyrano[3,2-b]pyridin-4-yl)-1H-indazole-6-carboxamide;
(S)—N-(4-(4-(trifluoromethyl)phenyl)-3,4-dihydro-2H-pyrano[3,2-b]pyridin-4-yl)pyrimidine-4-carboxamide;
(S)—N-(4-(4-(trifluoromethyl)phenyl)-3,4-dihydro-2H-pyrano[3,2-b]pyridin-4-yl)-1H-indole-6-carboxamide;
(S)—N-(4-(4-(trifluoromethyl)phenyl)-3,4-dihydro-2H-pyrano[3,2-b]pyridin-4-yl)pyridazine-4-carboxamide;
(S)-1-methyl-N-(4-(4-(trifluoromethyl)phenyl)-3,4-dihydro-2H-pyrano[3,2-b]pyridin-4-yl)-1H-indazole-6-carboxamide;
(S)—N-(4-(4-(trifluoromethyl)phenyl)-3,4-dihydro-2H-pyrano[3,2-b]pyridin-4-yl)thiazole-5-carboxamide;
(S)-5-hydroxy-N-(4-(4-(trifluoromethyl)phenyl)-3,4-dihydro-2H-pyrano[3,2-b]pyridin-4-yl)picolinamide;
(S)—N-(4-(4-(trifluoromethyl)phenyl)-3,4-dihydro-2H-pyrano[3,2-b]pyridin-4-yl)isothiazole-5-carboxamide;
(S)-1-methyl-N-(4-(4-(trifluoromethyl)phenyl)-3,4-dihydro-2H-pyrano[3,2-b]pyridin-4-yl)-1H-indole-5-carboxamide;
(S)-5-acetamido-N-(4-(4-(trifluoromethyl)phenyl)-3,4-dihydro-2H-pyrano[3,2-b]pyridin-4-yl)picolinamide;
(S)-1-methyl-N-(4-(4-(trifluoromethyl)phenyl)-3,4-dihydro-2H-pyrano[3,2-b]pyridin-4-yl)-1H-indazole-5-carboxamide;
(S)-5-oxo-N-(4-(4-(trifluoromethyl)phenyl)-3,4-dihydro-2H-pyrano[3,2-b]pyridin-4-yl)-4,5-dihydropyrazine-2-carboxamide;
(S)—N-(4-(4-(trifluoromethyl)phenyl)-3,4-dihydro-2H-pyrano[3,2-b]pyridin-4-yl)isonicotinamide;
(S)-6-methoxy-N-(4-(4-(trifluoromethyl)phenyl)-3,4-dihydro-2H-pyrano[3,2-b]pyridin-4-yl)pyridazine-3-carboxamide;
(S)—N-(4-(4-(trifluoromethyl)phenyl)-3,4-dihydro-2H-pyrano[3,2-b]pyridin-4-yl)nicotinamide;
(S)—N-(4-(4-(trifluoromethyl)phenyl)-3,4-dihydro-2H-pyrano[3,2-b]pyridin-4-yl)imidazo[1,2-a]pyridine-7-carboxamide;
(S)—N-(4-(3-fluoro-4-(trifluoromethoxy)phenyl)-3,4-dihydro-2H-pyrano[3,2-b]pyridin-4-yl)-5-oxo-4,5-dihydropyrazine-2-carboxamide;
(S)—N-(4-(3-fluoro-4-(trifluoromethoxy)phenyl)-3,4-dihydro-2H-pyrano[3,2-b]pyridin-4-yl)-5-hydroxypicolinamide;
(S)—N-(4-(3-fluoro-4-(trifluoromethoxy)phenyl)-3,4-dihydro-2H-pyrano[3,2-b]pyridin-4-yl)imidazo[1,2-a]pyridine-6-carboxamide;
(S)—N-(4-(3-fluoro-4-(trifluoromethoxy)phenyl)-3,4-dihydro-2H-pyrano[3,2-b]pyridin-4-yl)-1-methyl-1H-indazole-5-carboxamide;
(S)—N-(4-(3-fluoro-4-(trifluoromethoxy)phenyl)-3,4-dihydro-2H-pyrano[3,2-b]pyridin-4-yl)-2-oxo-1,2,3,4-tetrahydroquinoline-6-carboxamide;
(S)-5-fluoro-N-(4-(3-fluoro-4-(trifluoromethoxy)phenyl)-3,4-dihydro-2H-pyrano[3,2-b]pyridin-4-yl)picolinamide;
(S)—N-(4-(4-(trifluoromethyl)phenyl)-3,4-dihydro-2H-pyrano[3,2-b]pyridin-4-yl)quinoline-7-carboxamide;
(S)—N-(4-(3-fluoro-4-(trifluoromethoxy)phenyl)-3,4-dihydro-2H-pyrano[3,2-b]pyridin-4-yl)-2-oxo-2,3-dihydrobenzo[d]oxazole-5-carboxamide;
(S)-6-oxo-N-(4-(4-(trifluoromethoxy)phenyl)-3,4-dihydro-2H-pyrano[3,2-b]pyridin-4-yl)-1,6-dihydropyridine-3-carboxamide;
(S)—N-(4-(3-fluoro-4-(trifluoromethoxy)phenyl)-3,4-dihydro-2H-pyrano[3,2-b]pyridin-4-yl)-6-oxo-1,6-dihydropyridine-3-carboxamide;
(S)-6-oxo-N-(4-(4-(trifluoromethyl)phenyl)-3,4-dihydro-2H-pyrano[3,2-b]pyridin-4-yl)-1,6-dihydropyridine-3-carboxamide;
(S)—N-(4-(3-fluoro-4-(trifluoromethoxy)phenyl)-3,4-dihydro-2H-pyrano[3,2-b]pyridin-4-yl)-5-nitropicolinamide;
(S)—N5-(4-(3-fluoro-4-(trifluoromethoxy)phenyl)-3,4-dihydro-2H-pyrano[3,2-b]pyridin-4-yl)-N2-methylpyridine-2,5-dicarboxamide;
(S)—N-(4-(3-fluoro-4-(trifluoromethoxy)phenyl)-3,4-dihydro-2H-pyrano[3,2-b]pyridin-4-yl)-2,3-dioxo-1,2,3,4-tetrahydroquinoxaline-6-carboxamide;
(S)—N-(4-(3-fluoro-4-(trifluoromethoxy)phenyl)-3,4-dihydro-2H-pyrano[3,2-b]pyridin-4-yl)-5-(methylsulfonamido)picolinamide;
(S)-1-(4-(3-fluoro-4-(trifluoromethoxy)phenyl)-3,4-dihydro-2H-pyrano[3,2-b]pyridin-4-yl)-3-(6-fluoropyridin-3-yl)urea;
1-((S)-4-(4-(trifluoromethyl)phenyl)-3,4-dihydro-2H-pyrano[3,2-b]pyridin-4-yl)-3-((S)-1,1,1-trifluoropropan-2-yl)urea;
(S)-1-(4-cyanophenyl)-3-(4-(3-fluoro-4-(trifluoromethoxy)phenyl)-3,4-dihydro-2H-pyrano[3,2-b]pyridin-4-yl)urea;
(S)-1-(6-chloropyridin-3-yl)-3-(4-(3-fluoro-4-(trifluoromethoxy)phenyl)-3,4-dihydro-2H-pyrano[3,2-b]pyridin-4-yl)urea;
1-(pyridin-3-yl)-3-(4-(4-(trifluoromethyl)phenyl)chroman-4-yl)urea;
1-(pyridin-3-yl)-3-(8-(4-(trifluoromethyl)phenyl)-5,6,7,8-tetrahydroquinolin-8-yl)urea;
(S)-1-(3-fluorophenyl)-3-(4-(4-(trifluoromethyl)phenyl)-3,4-dihydro-2H-pyrano[3,2-b]pyridin-4-yl)urea;
(S)-1-(pyridin-3-yl)-3-(4-(4-(trifluoromethyl)phenyl)-3,4-dihydro-2H-pyrano[3,2-b]pyridin-4-yl)urea;
(S)-1-(4-fluorophenyl)-3-(4-(4-(trifluoromethyl)phenyl)-3,4-dihydro-2H-pyrano[3,2-b]pyridin-4-yl)urea;
(S)-6-((4-(3-fluoro-4-(trifluoromethoxy)phenyl)-3,4-dihydro-2H-pyrano[3,2-b]pyridin-4-yl)carbamoyl)nicotinic acid;
(S)—N-(4-(3-fluoro-4-(trifluoromethoxy)phenyl)-3,4-dihydro-2H-pyrano[3,2-b]pyridin-4-yl)benzenesulfonamide;
(S)—N-(4-(3-fluoro-4-(trifluoromethoxy)phenyl)-3,4-dihydro-2H-pyrano[3,2-b]pyridin-4-yl)pyridine-3-sulfonamide; or
(S)-tert-butyl (4-(3-fluoro-4-(trifluoromethoxy)phenyl)-3,4-dihydro-2H-pyrano[3,2-b]pyridin-4-yl)carbamate; or
   the pharmaceutically-acceptable salt thereof, the tautomer thereof, the pharmaceutically-acceptable salt of the tautomer, the stereoisomer thereof, or the mixture thereof.

80. The compound of embodiment 1, wherein the compound is
(S)—N-(4-(4-(trifluoromethyl)phenyl)-3,4-dihydro-2H-pyrano[3,2-b]pyridin-4-yl)benzamide;
(S)—N-(4-(3,4-dichlorophenyl)-3,4-dihydro-2H-pyrano[3,2-b]pyridin-4-yl)benzamide;
(S)-4-hydroxy-N-(4-(4-(trifluoromethyl)phenyl)-3,4-dihydro-2H-pyrano[3,2-b]pyridin-4-yl)benzamide;
(S)-4-methoxy-N-(4-(4-(trifluoromethyl)phenyl)-3,4-dihydro-2H-pyrano[3,2-b]pyridin-4-yl)benzamide;
4-fluoro-N-(4-(3-fluoro-4-(trifluoromethoxy)phenyl)-3,4-dihydro-2H-pyrano[3,2-b]pyridin-4-yl)benzamide;
N-(4-(3-fluoro-4-(trifluoromethoxy)phenyl)-3,4-dihydro-2H-pyrano[3,2-b]pyridin-4-yl)benzamide;
N-(4-(4-(trifluoromethoxy)phenyl)-3,4-dihydro-2H-pyrano[3,2-b]pyridin-4-yl)benzamide;
(S)—N-(4-(3-fluoro-4-(trifluoromethoxy)phenyl)-3,4-dihydro-2H-pyrano[3,2-b]pyridin-4-yl)-2-oxo-2,3-dihydrobenzo[d]oxazole-6-carboxamide;
2-oxo-N-(4-(4-(trifluoromethoxy)phenyl)-3,4-dihydro-2H-pyrano[3,2-b]pyridin-4-yl)-2,3-dihydrobenzo[d]oxazole-6-carboxamide;
2-oxo-N-(4-(4-(trifluoromethoxy)phenyl)-3,4-dihydro-2H-pyrano[3,2-b]pyridin-4-yl)-2,3-dihydrobenzo[d]oxazole-5-carboxamide;
N-(4-(3,4-dichlorophenyl)-3,4-dihydro-2H-pyrano[3,2-b]pyridin-4-yl)-2-oxo-2,3-dihydrobenzo[d]oxazole-6-carboxamide;
N-(4-(3,4-dichlorophenyl)-3,4-dihydro-2H-pyrano[3,2-b]pyridin-4-yl)-2-oxo-2,3-dihydrobenzo[d]oxazole-5-carboxamide;
2-oxo-N-(4-phenyl-3,4-dihydro-2H-pyrano[3,2-b]pyridin-4-yl)-2,3-dihydrobenzo[d]oxazole-6-carboxamide;
methyl 6-((4-(3-fluoro-4-(trifluoromethoxy)phenyl)-3,4-dihydro-2H-pyrano[3,2-b]pyridin-4-yl)carbamoyl)nicotinate;
(S)—N-(4-(4-(trifluoromethyl)phenyl)-3,4-dihydro-2H-pyrano[3,2-b]pyridin-4-yl)-1H-indazole-6-carboxamide;
(S)—N-(4-(4-(trifluoromethyl)phenyl)-3,4-dihydro-2H-pyrano[3,2-b]pyridin-4-yl)-1H-indole-6-carboxamide;
(S)-1-methyl-N-(4-(4-(trifluoromethyl)phenyl)-3,4-dihydro-2H-pyrano[3,2-b]pyridin-4-yl)-1H-indazole-6-carboxamide;
(S)—N-(4-(4-(trifluoromethyl)phenyl)-3,4-dihydro-2H-pyrano[3,2-b]pyridin-4-yl)thiazole-5-carboxamide;
(S)-5-hydroxy-N-(4-(4-(trifluoromethyl)phenyl)-3,4-dihydro-2H-pyrano[3,2-b]pyridin-4-yl)picolinamide;
(S)-1-methyl-N-(4-(4-(trifluoromethyl)phenyl)-3,4-dihydro-2H-pyrano[3,2-b]pyridin-4-yl)-1H-indole-5-carboxamide;
(S)-5-oxo-N-(4-(4-(trifluoromethyl)phenyl)-3,4-dihydro-2H-pyrano[3,2-b]pyridin-4-yl)-4,5-dihydropyrazine-2-carboxamide;
(S)—N-(4-(3-fluoro-4-(trifluoromethoxy)phenyl)-3,4-dihydro-2H-pyrano[3,2-b]pyridin-4-yl)imidazo[1,2-a]pyridine-6-carboxamide;
(S)—N-(4-(3-fluoro-4-(trifluoromethoxy)phenyl)-3,4-dihydro-2H-pyrano[3,2-b]pyridin-4-yl)-1-methyl-1H-indazole-5-carboxamide;
(S)—N-(4-(3-fluoro-4-(trifluoromethoxy)phenyl)-3,4-dihydro-2H-pyrano[3,2-b]pyridin-4-yl)-2-oxo-1,2,3,4-tetrahydroquinoline-6-carboxamide;
(S)-5-fluoro-N-(4-(3-fluoro-4-(trifluoromethoxy)phenyl)-3,4-dihydro-2H-pyrano[3,2-b]pyridin-4-yl)picolinamide;
(S)—N-(4-(4-(trifluoromethyl)phenyl)-3,4-dihydro-2H-pyrano[3,2-b]pyridin-4-yl)quinoline-7-carboxamide;
(S)—N-(4-(3-fluoro-4-(trifluoromethoxy)phenyl)-3,4-dihydro-2H-pyrano[3,2-b]pyridin-4-yl)-2-oxo-2,3-dihydrobenzo[d]oxazole-5-carboxamide;
(S)-6-oxo-N-(4-(4-(trifluoromethoxy)phenyl)-3,4-dihydro-2H-pyrano[3,2-b]pyridin-4-yl)-1,6-dihydropyridine-3-carboxamide;
(S)—N-(4-(3-fluoro-4-(trifluoromethoxy)phenyl)-3,4-dihydro-2H-pyrano[3,2-b]pyridin-4-yl)-6-oxo-1,6-dihydropyridine-3-carboxamide;
(S)—N5-(4-(3-fluoro-4-(trifluoromethoxy)phenyl)-3,4-dihydro-2H-pyrano[3,2-b]pyridin-4-yl)-N2-methylpyridine-2,5-dicarboxamide;
(S)—N-(4-(3-fluoro-4-(trifluoromethoxy)phenyl)-3,4-dihydro-2H-pyrano[3,2-b]pyridin-4-yl)-5-(methylsulfonamido)picolinamide;
(S)-1-(4-(3-fluoro-4-(trifluoromethoxy)phenyl)-3,4-dihydro-2H-pyrano[3,2-b]pyridin-4-yl)-3-(6-fluoropyridin-3-yl)urea;
(S)-1-(4-cyanophenyl)-3-(4-(3-fluoro-4-(trifluoromethoxy)phenyl)-3,4-dihydro-2H-pyrano[3,2-b]pyridin-4-yl)urea;
(S)-1-(6-chloropyridin-3-yl)-3-(4-(3-fluoro-4-(trifluoromethoxy)phenyl)-3,4-dihydro-2H-pyrano[3,2-b]pyridin-4-yl)urea;
(S)-1-(pyridin-3-yl)-3-(4-(4-(trifluoromethyl)phenyl)-3,4-dihydro-2H-pyrano[3,2-b]pyridin-4-yl)urea;
(S)-1-(4-fluorophenyl)-3-(4-(4-(trifluoromethyl)phenyl)-3,4-dihydro-2H-pyrano[3,2-b]pyridin-4-yl)urea; or
(S)-6-((4-(3-fluoro-4-(trifluoromethoxy)phenyl)-3,4-dihydro-2H-pyrano[3,2-b]pyridin-4-yl)carbamoyl)nicotinic acid; or
   the pharmaceutically-acceptable salt thereof, the tautomer thereof, the pharmaceutically-acceptable salt of the tautomer, the stereoisomer thereof, or the mixture thereof.

81. The compound of embodiment 1, wherein the compound is (S)—N-(4-(4-(trifluoromethyl)phenyl)-3,4-dihydro-2H-pyrano[3,2-b]pyridin-4-yl)benzamide or the pharmaceutically-acceptable salt thereof, the tautomer thereof, the pharmaceutically-acceptable salt of the tautomer, the stereoisomer thereof, or the mixture thereof.

82. The compound of embodiment 1, wherein the compound is (S)-4-hydroxy-N-(4-(4-(trifluoromethyl)phenyl)-3,4-dihydro-2H-pyrano[3,2-b]pyridin-4-yl)benzamide or the pharmaceutically-acceptable salt thereof, the tautomer thereof, the pharmaceutically-acceptable salt of the tautomer, the stereoisomer thereof, or the mixture thereof.

83. The compound of embodiment 1, wherein the compound is (S)-6-((4-(3-fluoro-4-(trifluoromethoxy)phenyl)-3,4-dihydro-2H-pyrano[3,2-b]pyridin-4-yl)carbamoyl)nicotinic acid or the pharmaceutically-acceptable salt thereof, the tautomer thereof, the pharmaceutically-acceptable salt of the tautomer, the stereoisomer thereof, or the mixture thereof.

84. The compound of embodiment 1, wherein the compound is (S)—N-(4-(3-fluoro-4-(trifluoromethoxy)phenyl)-3,4-dihydro-2H-pyrano[3,2-b]pyridin-4-yl)-2-oxo-2,3-dihydrobenzo[d]oxazole-6-carboxamide or the pharmaceutically-acceptable salt thereof, the tautomer thereof, the pharmaceutically-acceptable salt of the tautomer, the stereoisomer thereof, or the mixture thereof.

85. The compound of embodiment 1, wherein the compound is 2-oxo-N-(4-(4-(trifluoromethoxy)phenyl)-3,4-dihydro-2H-pyrano[3,2-b]pyridin-4-yl)-2,3-dihydrobenzo[d]oxazole-6-carboxamide or the pharmaceutically-acceptable salt thereof, the tautomer thereof, the pharmaceutically-acceptable salt of the tautomer, the stereoisomer thereof, or the mixture thereof.

86. The compound of embodiment 1, wherein the compound is 2-oxo-N-(4-(4-(trifluoromethoxy)phenyl)-3,4-dihydro-2H-pyrano[3,2-b]pyridin-4-yl)-2,3-dihydrobenzo[d]oxazole-5-carboxamide or the pharmaceutically-acceptable salt thereof, the tautomer thereof, the pharmaceutically-acceptable salt of the tautomer, the stereoisomer thereof, or the mixture thereof.

87. The compound of embodiment 1, wherein the compound is (S)—N-(4-(3-fluoro-4-(trifluoromethoxy)phenyl)-3,4-dihydro-2H-pyrano[3,2-b]pyridin-4-yl)-6-oxo-1,6-dihydropyridine-3-carboxamide or the pharmaceutically-acceptable salt thereof, the tautomer thereof, the pharmaceutically-acceptable salt of the tautomer, the stereoisomer thereof, or the mixture thereof.

88. The compound of embodiment 1, wherein the compound is N-(4-(3,4-dichlorophenyl)-3,4-dihydro-2H-pyrano[3,2-b]pyridin-4-yl)-2-oxo-2,3-dihydrobenzo[d]oxazole-5-carboxamide or the pharmaceutically-acceptable salt thereof, the tautomer thereof, the pharmaceutically-acceptable salt of the tautomer, the stereoisomer thereof, or the mixture thereof.

89. The compound of embodiment 1, wherein the compound is 2-oxo-N-(4-phenyl-3,4-dihydro-2H-pyrano[3,2-b]pyridin-4-yl)-2,3-dihydrobenzo[d]oxazole-6-carboxamide or the pharmaceutically-acceptable salt thereof, the tautomer thereof, the pharmaceutically-acceptable salt of the tautomer, the stereoisomer thereof, or the mixture thereof.

90. The compound of embodiment 1, wherein the compound is (S)-1-(pyridin-3-yl)-3-(4-(4-(trifluoromethyl)phenyl)-3,4-dihydro-2H-pyrano[3,2-b]pyridin-4-yl)urea or the pharmaceutically-acceptable salt thereof, the tautomer thereof, the pharmaceutically-acceptable salt of the tautomer, the stereoisomer thereof, or the mixture thereof.

91. The compound of embodiment 1, wherein the compound is (S)—N-(4-(3-fluoro-4-(trifluoromethoxy)phenyl)-3,4-dihydro-2H-pyrano[3,2-b]pyridin-4-yl)-5-(methylsulfonamido)picolinamide or the pharmaceutically-acceptable salt thereof, the tautomer thereof, the pharmaceutically-acceptable salt of the tautomer, the stereoisomer thereof, or the mixture thereof.

92. The compound of embodiment 1, wherein the compound is (S)—N5-(4-(3-fluoro-4-(trifluoromethoxy)phenyl)-3,4-dihydro-2H-pyrano[3,2-b]pyridin-4-yl)-N2-methylpyridine-2,5-dicarboxamide or the pharmaceutically-acceptable salt thereof, the tautomer thereof, the pharmaceutically-acceptable salt of the tautomer, the stereoisomer thereof, or the mixture thereof.

93. The compound of embodiment 1, wherein the compound is (S)—N-(4-(3-fluoro-4-(trifluoromethoxy)phenyl)-3,4-dihydro-2H-pyrano[3,2-b]pyridin-4-yl)-2-oxo-2,3-dihydrobenzo[d]oxazole-5-carboxamide or the pharmaceutically-acceptable salt thereof, the tautomer thereof, the pharmaceutically-acceptable salt of the tautomer, the stereoisomer thereof, or the mixture thereof.

94. The compound of embodiment 1, wherein the compound is (S)-5-hydroxy-N-(4-(4-(trifluoromethyl)phenyl)-3,4-dihydro-2H-pyrano[3,2-b]pyridin-4-yl)picolinamide or the pharmaceutically-acceptable salt thereof, the tautomer thereof, the pharmaceutically-acceptable salt of the tautomer, the stereoisomer thereof, or the mixture thereof.

95. The compound of embodiment 1, wherein the compound is (S)-2-oxo-N-(4-(4-(trifluoromethyl)phenyl)-3,4-dihydro-2H-pyrano[3,2-b]pyridin-4-yl)-1,2,3,4-tetrahydroquinoline-6-carboxamide or the pharmaceutically-acceptable salt thereof, the tautomer thereof, the pharmaceutically-acceptable salt of the tautomer, the stereoisomer thereof, or the mixture thereof.

96. The compound of embodiment 1, wherein the compound is methyl 6-((4-(3-fluoro-4-(trifluoromethoxy)phenyl)-3,4-dihydro-2H-pyrano[3,2-b]pyridin-4-yl)carbamoyl)nicotinate or the pharmaceutically-acceptable salt thereof, the tautomer thereof, the pharmaceutically-acceptable salt of the tautomer, the stereoisomer thereof, or the mixture thereof.

97. The compound of embodiment 1, wherein the compound is
(S)—N-(4-(3,4-dichlorophenyl)-3,4-dihydro-2H-pyrano[3,2-b]pyridin-4-yl)-6-oxo-1,6-dihydropyridine-3-carboxamide;
(S)—N-(4-(2-fluoro-4-(trifluoromethoxy)phenyl)-3,4-dihydro-2H-pyrano[3,2-b]pyridin-4-yl)-6-oxo-1,6-dihydropyridine-3-carboxamide;
(S)—N-(4-(2-chloro-4-(trifluoromethoxy)phenyl)-3,4-dihydro-2H-pyrano[3,2-b]pyridin-4-yl)-6-oxo-1,6-dihydropyridine-3-carboxamide;
(S)—N-(4-(2-methyl-4-(trifluoromethoxy)phenyl)-3,4-dihydro-2H-pyrano[3,2-b]pyridin-4-yl)-6-oxo-1,6-dihydropyridine-3-carboxamide;
(S)—N-(4-(3-chloro-4-(trifluoromethyl)phenyl)-3,4-dihydro-2H-pyrano[3,2-b]pyridin-4-yl)-6-oxo-1,6-dihydropyridine-3-carboxamide;
(S)—N-(4-(3-chloro-4-(trifluoromethoxy)phenyl)-3,4-dihydro-2H-pyrano[3,2-b]pyridin-4-yl)-6-oxo-1,6-dihydropyridine-3-carboxamide;
(S)—N-(4-(3-fluoro-4-(trifluoromethyl)phenyl)-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-6-methyl-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-7-methyl-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-8-methyl-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-6-chloro-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-7-chloro-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-8-chloro-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-6-fluoro-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-7-fluoro-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-8-fluoro-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-6-methoxy-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-7-methoxy-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-8-methoxy-3,4-dihydro-2H-pyrano[3,2-b]pyridin-4-yl)-6-oxo-1,6-dihydropyridine-3-carboxamide;
(S)—N-(4-([1,1′-biphenyl]-4-yl)-3,4-dihydro-2H-pyrano[3,2-b]pyridin-4-yl)-6-oxo-1,6-dihydropyridine-3-carboxamide;
(S)—N-(4-(3-fluoro-4-(trifluoromethoxy)phenyl)-2,2-dimethyl-3,4-dihydro-2H-pyrano[3,2-b]pyridin-4-yl)-6-oxo-1,6-dihydropyridine-3-carboxamide;
(S)—N-(4′-(3-fluoro-4-(trifluoromethoxy)phenyl)-3′,4′-dihydrospiro[cyclobutane-1,2′-pyrano[3,2-b]pyridin]-4′-yl)-6-oxo-1,6-dihydropyridine-3-carboxamide;
(S)—N-(4-(3-fluoro-4-(trifluoromethoxy)phenyl)chroman-4-yl)-6-oxo-1,6-dihydropyridine-3-carboxamide;
(S)—N-(8-(3-fluoro-4-(trifluoromethoxy)phenyl)-5,6,7,8-tetrahydroquinolin-8-yl)-6-oxo-1,6-dihydropyridine-3-carboxamide;
(S)—N-(4-(3-fluoro-4-(trifluoromethoxy)phenyl)-1-methyl-1,2,3,4-tetrahydro-1,5-naphthyridin-4-yl)-6-oxo-1,6-dihydropyridine-3-carboxamide;
(S)—N-(1-acetyl-4-(3-fluoro-4-(trifluoromethoxy)phenyl)-1,2,3,4-tetrahydro-1,5-naphthyridin-4-yl)-6-oxo-1,6-dihydropyridine-3-carboxamide;
(S)—N-(9-(3-fluoro-4-(trifluoromethoxy)phenyl)-6,7,8,9-tetrahydrooxepino[3,2-b]pyridin-9-yl)-6-oxo-1,6-dihydropyridine-3-carboxamide;
(S)—N-(3-(3-fluoro-4-(trifluoromethoxy)phenyl)-2,3-dihydrofuro[3,2-b]pyridin-3-yl)-6-oxo-1,6-dihydropyridine-3-carboxamide;
(S)—N-(4-(5-fluoro-6-(trifluoromethoxy)pyridin-3-yl)-3,4-dihydro-2H-pyrano[3,2-b]pyridin-4-yl)-6-oxo-1,6-dihydropyridine-3-carboxamide;
(S)-6-((4-(3,4-dichlorophenyl)-3,4-dihydro-2H-pyrano[3,2-b]pyridin-4-yl)carbamoyl)nicotinic acid;
(S)-6-((4-(4-(trifluoromethoxy)phenyl)-3,4-dihydro-2H-pyrano[3,2-b]pyridin-4-yl)carbamoyl)nicotinic acid;
(S)-6-((4-(4-(trifluoromethyl)phenyl)-3,4-dihydro-2H-pyrano[3,2-b]pyridin-4-yl)carbamoyl)nicotinic acid;
(S)-6-((4-(3-fluoro-4-(trifluoromethyl)phenyl)-3,4-dihydro-2H-pyrano[3,2-b]pyridin-4-yl)carbamoyl)nicotinic acid;
(S)-6-((4-(3-chloro-4-(trifluoromethyl)phenyl)-3,4-dihydro-2H-pyrano[3,2-b]pyridin-4-yl)carbamoyl)nicotinic acid;
(S)-6-((4-(2-fluoro-4-(trifluoromethoxy)phenyl)-3,4-dihydro-2H-pyrano[3,2-b]pyridin-4-yl)carbamoyl)nicotinic acid;
(S)-6-((4-(3-chloro-4-(trifluoromethoxy)phenyl)-3,4-dihydro-2H-pyrano[3,2-b]pyridin-4-yl)carbamoyl)nicotinic acid;
(S)-6-((4-(3-fluoro-4-(trifluoromethoxy)phenyl)chroman-4-yl)carbamoyl)nicotinic acid;
(S)-6-((8-(3-fluoro-4-(trifluoromethoxy)phenyl)-5,6,7,8-tetrahydroquinolin-8-yl)carbamoyl)nicotinic acid;
(S)—N-(4-(3-fluoro-4-(trifluoromethoxy)phenyl)-1,2,3,4-tetrahydro-1,5-naphthyridin-4-yl)-6-oxo-1,6-dihydropyridine-3-carboxamide;
(S)—N-(9-(3-fluoro-4-(trifluoromethoxy)phenyl)-6,7,8,9-tetrahydro-5H-pyrido[3,2-b]azepin-9-yl)-6-oxo-1,6-dihydropyridine-3-carboxamide; or
(R)—N-(3-(3-fluoro-4-(trifluoromethoxy)phenyl)-2,3-dihydro-1H-pyrrolo[3,2-b]pyridin-3-yl)-6-oxo-1,6-dihydropyridine-3-carboxamide; or
   the pharmaceutically-acceptable salt thereof, the tautomer thereof, the pharmaceutically-acceptable salt of the tautomer, the stereoisomer thereof, or the mixture thereof.

98. The compound or tautomer of any one of embodiments 1-97 in a neutral form.

99. The compound of any one of embodiments 1-97 in a neutral form.

100. The pharmaceutically-acceptably salt of the compound or the pharmaceutically acceptable salt of the tautomer of any one of embodiments 1-97.

101. The pharmaceutically-acceptably salt of the compound of any one of embodiments 1-97.

102. A pharmaceutical composition comprising the compound according to any one of embodiments 1-97 or the pharmaceutically-acceptable salt thereof, the tautomer thereof, the pharmaceutically-acceptable salt of the tautomer, the stereoisomer thereof, or the mixture thereof, and a pharmaceutically-acceptable diluent or carrier.

103. A method of treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders in a subject, the method comprising administering the compound according to any one of embodiments 1-96 or the pharmaceutically-acceptable salt thereof, the tautomer thereof, the pharmaceutically-acceptable salt of the tautomer, the stereoisomer thereof, or the mixture thereof to the subject.

104. The method of embodiment 103, wherein the subject is suffering from neuropathic pain.

105. The method of embodiment 103, wherein the subject is suffering from migraine pain.

106. The use of the compound according to any one of embodiments 1-97 or the pharmaceutically-acceptable salt thereof, the tautomer thereof, the pharmaceutically-acceptable salt of the tautomer, the stereoisomer thereof, or the mixture thereof in the preparation of a medicament.

107. The use of the compound according to any one of embodiments 1-97 or the pharmaceutically-acceptable salt thereof, the tautomer thereof, the pharmaceutically-acceptable salt of the tautomer, the stereoisomer thereof, or the mixture thereof for treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders in a subject.

108. The use of embodiment 107, wherein the use is for treating neuropathic pain.

109. The use of embodiment 107, wherein the use is for treating migraine.

110. The compound according to any one of embodiments 1-97 or the pharmaceutically-acceptable salt thereof, the tautomer thereof, the pharmaceutically-acceptable salt of the tautomer, the stereoisomer thereof, or the mixture thereof for treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders in a subject.

111. The compound of embodiment 110 or the pharmaceutically-acceptable salt thereof, the tautomer thereof, the pharmaceutically-acceptable salt of the tautomer, the stereoisomer thereof, or the mixture thereof for treating neuropathic pain in a subject.

112. The compound of embodiment 110 or the pharmaceutically-acceptable salt thereof, the tautomer thereof, the pharmaceutically-acceptable salt of the tautomer, the stereoisomer thereof, or the mixture thereof for treating migraine in a subject.

113. In a 113th embodiment, the invention provides a compound of Formula I having the following structure:

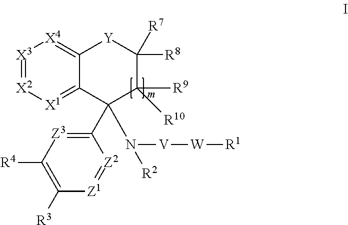

a pharmaceutically-acceptable salt thereof, a tautomer thereof, a pharmaceutically-acceptable salt of the tautomer, a stereoisomer thereof, or a mixture thereof, wherein:

V is selected from —C(═O)— or —S(═O)₂—;

W is absent or is selected from —NH—, —NR^{1a}—, or O;

X¹ is selected from —CR⁵— or —N—;

X² is selected from —CR⁵— or —N—;

X³ is selected from —CR⁵— or —N—;

X⁴ is selected from —CR⁵— or —N—;

Y is selected from —O—, —CH₂—, —NH—, —NR^{1b}—, —CF₂—, —C(═O)—, —C(H)(F)—, or —C(H)(OH)—;

Z¹ is selected from —CR⁶— or —N—;

Z² is selected from —CR⁶— or —N—;

Z³ is selected from —CR⁶— or —N—;

wherein 0, 1, or 2 of X¹, X², X³, and X⁴ are N;

wherein 0, 1, or 2 of Z¹, Z², and Z³ are N;

m is 0, 1, or 2;

R¹ is C₁₋₆alk or a direct-bonded, C₁₋₂alk-linked, C₁₋₂alkO-linked, saturated, partially-saturated or unsaturated 3-, 4-, 5-, 6- or 7-membered monocyclic or 7-, 8-, 9-, 10- or 11-membered bicyclic ring containing 0, 1, 2, 3 or 4 heteroatoms selected from N, O and S, but containing no more than one O or S atom, the C₁₋₆alk and ring being substituted by 0, 1, 2 or 3 substituents independently selected from halo, oxo, C₁₋₆alk, C₁₋₆alkOH, C₁₋₆alkOH substituted by 1, 2, or 3 halo substituents, C₁₋₆alk-C(═O)R^{a}, C₁₋₆alk-C(═O)OR^{a}, C₁₋₄haloalk, cyano, nitro, —C(═O)R^{a}, —C(═O)OR^{a}, —C(═O)NR^{a}R^{a}, —C(═O)NR^{a}S(═O)₂R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, ═S, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the ring is additionally substituted by 0 or 1 directly bonded, SO₂ linked, C(═O) linked or CH₂ linked saturated, partially-saturated or unsaturated 3-, 4-, 5-, 6- or 7-membered monocyclic ring containing 0, 1, 2, 3 or 4 heteroatoms selected from N, O and S, but containing no more than one O or S atom, and substituted by 0, 1, 2 or 3 groups selected from halo, oxo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —SR^{a}, —S(═O)R^{a}, —S(═O)₂R^{a}, —S(═O)₂NR^{a}R^{a}, —NR^{a}R^{a}, and —N(R^{a})C(═O)R^{a};

R^{1a} is C₁₋₆alk;

R^{1b} is C₁₋₆alk or —C(═O)R^{b};

R² is H or C₁₋₆alk;

R³ is H, C₁₋₈alk, C₁₋₈alkOH, C₁₋₄haloalk, halo, cyano, R^{b}, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} or —NR^{a}C₂₋₆alkOR^{a};

R⁴ is H, C₁₋₆alk, —C₁₋₃haloalk, —OC₁₋₆alk, —OC₁₋₃haloalk, —N(C₁₋₆alk)C₁₋₆alk, —NHC₁₋₆alk, —NC(═O)C₁₋₆alk, —N(C₁₋₆alk)C₁₋₆alk, F, Cl, Br, CN, OH or NH₂; or R³ and R⁴ together form a four-atom unsaturated bridge containing 0 or 1 N atoms, wherein the bridge is substituted by 0, 1 or 2 R⁵ substituents;

R⁵ is, at each instance, independently selected from H, C₁₋₈alk, C₁₋₈alkOH, C₁₋₄haloalk, halo, cyano, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(Ra)C(═O)OR^{b}, —N(R^{a})C(═O)NR^{a}R^{a}, —N(Ra)C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} or —NR^{a}C₂₋₆alkOR^{a};

R⁶ is, at each instance, independently selected from H, halo, OR^{a}, C₁₋₆alk, or CF₃;

R⁷ and R⁸ are independently selected from H or C₁₋₆alk, or R⁷ and R⁸, together with the carbon atom to which they are attached, join to form a 3 to 7 membered cycloalkyl ring or a 3-7 membered heterocyclyl ring that includes 1 or 2 heteroatoms selected from O, N, or S;

R⁹ and R¹⁰ are, at each instance, independently selected from H or C₁₋₆alk;

R^{a} is independently, at each instance, H or R^{b}; and

R^{b} is independently, at each instance, phenyl, benzyl or C₁₋₆alk, and when R³ is R^{b}, R^{b} may additionally be an unsaturated 5 or 6-membered monocyclic ring containing 1, 2, or 3 heteroatoms selected from N, O, and S, the phenyl, benzyl C₁₋₆alk, and unsaturated 5 or 6-membered monocyclic ring being substituted by 0, 1, 2 or 3 substituents selected from halo, cyano, oxo, C₁₋₄alk, C₁₋₄alkOH, C₁₋₃haloalk, —OC₁₋₄alk, —OH, —NH₂, —OC₁₋₄alk, —OC₁₋₄haloalk, —S(═O)₂C₁₋₄alk, —NHC(═O)—C₁₋₄alk, —C(═O)NH₂, —C(═O)NHC₁₋₄alk, —C(═O)N(C₁₋₄alk)₂, —NHC₁₋₄alk, and —N(C₁₋₄alk)C₁₋₄alk, or a saturated, partially-saturated, or unsaturated 5 or 6-membered monocyclic ring containing 1 or 2 heteroatoms selected from N, O, and S;

wherein the compound is not one of the following compounds and is not a salt thereof:

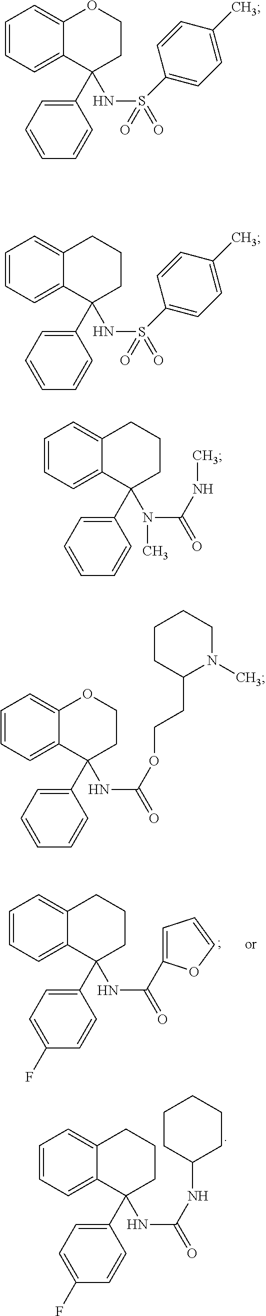

In some versions of embodiment 113, the compound is not one of the following compounds, is not a salt thereof, is not a tautomer thereof, is not a salt of a tautomer, is not a stereoisomer thereof, and is not a salt of a stereoisomer:

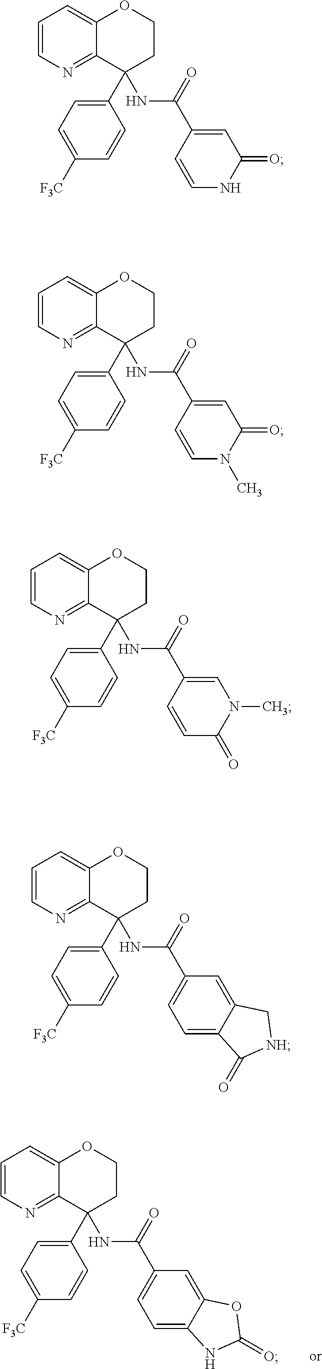 [SMILES: O=C(NC1(c2ccc(C(F)(F)F)cc2)CCOc2cccnc21)c1cc[nH]c(=O)c1 ; Cn1ccc(C(=O)NC2(c3ccc(C(F)(F)F)cc3)CCOc3cccnc32)cc1=O ; Cn1cc(C(=O)NC2(c3ccc(C(F)(F)F)cc3)CCOc3cccnc32)ccc1=O ; O=C(NC1(c2ccc(C(F)(F)F)cc2)CCOc2cccnc21)c1ccc2c(c1)CNC2=O ; O=C(NC1(c2ccc(C(F)(F)F)cc2)CCOc2cccnc21)c1ccc2[nH]c(=O)oc2c1]

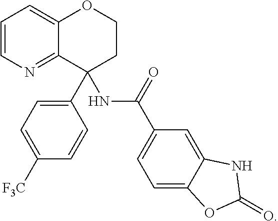 [SMILES: O=C(NC1(c2ccc(C(F)(F)F)cc2)CCOc2cccnc21)c1ccc2oc(=O)[nH]c2c1]

In some embodiments, Y is selected from —O—, —CH₂—, —NH—, or —NR^{1b}—. In still further such embodiments, Y is selected from —O— or —CH₂—. In some embodiments, X¹ is N; X², X³ and X⁴ are CR⁵; Y is O; m is 1; R⁷, R⁸, R⁹, and R¹⁰ are H; two or three of Z¹, Z², and Z³ are CR⁶; R² is H; V is —C(═O)—; W is absent or is —NH—; R³ is —CF₃ or —OCF₃; R⁴ is —F, —Cl, or —OCF₃; and R¹ is a partially-saturated or unsaturated 3-, 4-, 5-, 6- or 7-membered monocyclic or 7-, 8-, 9-, 10- or 11-membered bicyclic ring containing 0, 1, 2, 3 or 4 heteroatoms selected from N, O and S, but containing no more than one O or S atom, and the ring is substituted by 0, 1, 2 or 3 substituents independently selected from halo, oxo, C₁₋₆alk, C₁₋₆alkOH, C₁₋₆alkOH substituted by 1, 2, or 3 halo substituents, C₁₋₆alk-C(═O)R^{a}, C₁₋₆alk-C(═O)OR^{a}, C₁₋₄haloalk, cyano, nitro, —C(═O)R^{a}, —C(═O)OR^{a}, —C(═O)NR^{a}R^{a}, —C(═O)NR^{a}S(═O)₂R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the ring is additionally substituted by 0 or 1 directly bonded, SO₂ linked, C(═O) linked or CH₂ linked saturated, partially-saturated or unsaturated 3-, 4-, 5-, 6- or 7-membered monocyclic ring containing 0, 1, 2, 3 or 4 heteroatoms selected from N, O and S, but containing no more than one O or S atom, and substituted by 0, 1, 2 or 3 groups selected from halo, oxo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —SR^{a}, —S(═O)R^{a}, —S(═O)₂R^{a}, —S(═O)₂NR^{a}R^{a}, —NR^{a}R^{a}, and —N(R^{a})C(═O)R^{a}. In some such embodiments, R⁵ is H; Z¹, Z², and Z³ are each CR⁶; W is absent; and R⁴ is —F. In still further such embodiments, R³ is —CF₃ whereas in other such embodiments, R³ is —OCF₃. In still further such embodiments, R⁶ is H.

114. The compound of embodiment 113 or the pharmaceutically-acceptable salt thereof, the tautomer thereof, the pharmaceutically-acceptable salt of the tautomer, the stereoisomer thereof, or the mixture thereof, wherein R² and R⁸ are both H.

115. The compound of embodiment 113 or the pharmaceutically-acceptable salt thereof, the tautomer thereof, the pharmaceutically-acceptable salt of the tautomer, the stereoisomer thereof, or the mixture thereof, wherein R² and R⁸ are selected from H and —CH₃.

116. The compound of embodiment 115 or the pharmaceutically-acceptable salt thereof, the tautomer thereof, the pharmaceutically-acceptable salt of the tautomer, the stereoisomer thereof, or the mixture thereof, wherein R² and R⁸ are both —CH₃.

117. The compound of embodiment 113 or the pharmaceutically-acceptable salt thereof, the tautomer thereof, the pharmaceutically-acceptable salt of the tautomer, the stereoisomer thereof, or the mixture thereof, wherein R² and R⁸, together with the carbon atom to which they are attached, join to form a 3-7 membered cycloalkyl ring.

118. The compound of embodiment 117 or the pharmaceutically-acceptable salt thereof, the tautomer thereof, the pharmaceutically-acceptable salt of the tautomer, the stereoisomer thereof, or the mixture thereof, wherein R⁷ and R⁸, together with the carbon atom to which they are attached, join to form a cyclopropyl or cyclobutyl ring.

119. The compound of embodiment 113 or the pharmaceutically-acceptable salt thereof, the tautomer thereof, the pharmaceutically-acceptable salt of the tautomer, the stereoisomer thereof, or the mixture thereof, wherein m is 0.

120. The compound of any one of embodiments 113-118 or the pharmaceutically-acceptable salt thereof, the tautomer thereof, the pharmaceutically-acceptable salt of the tautomer, the stereoisomer thereof, or the mixture thereof, wherein m is 1.

121. The compound of any one of embodiment 113-118 or the pharmaceutically-acceptable salt thereof, the tautomer thereof, the pharmaceutically-acceptable salt of the tautomer, the stereoisomer thereof, or the mixture thereof, wherein m is 2.

122. The compound of any one of embodiments 113-121 or the pharmaceutically-acceptable salt thereof, the tautomer thereof, the pharmaceutically-acceptable salt of the tautomer, the stereoisomer thereof, or the mixture thereof, wherein Y is —NH—.

123. The compound of any one of embodiments 113-121 or the pharmaceutically-acceptable salt thereof, the tautomer thereof, the pharmaceutically-acceptable salt of the tautomer, the stereoisomer thereof, or the mixture thereof, wherein Y is —NR^{1b}— and R^{1b} is —CH₃.

124. The compound of any one of embodiments 113-121 or the pharmaceutically-acceptable salt thereof, the tautomer thereof, the pharmaceutically-acceptable salt of the tautomer, the stereoisomer thereof, or the mixture thereof, wherein Y is —O—.

125. The compound of any one of embodiments 113-121 or the pharmaceutically-acceptable salt thereof, the tautomer thereof, the pharmaceutically-acceptable salt of the tautomer, the stereoisomer thereof, or the mixture thereof, wherein Y is —CH₂—.

126. The compound of any one of embodiments 113-125 or the pharmaceutically-acceptable salt thereof, the tautomer thereof, the pharmaceutically-acceptable salt of the tautomer, the stereoisomer thereof, or the mixture thereof, wherein R⁹ and R¹⁰ are H.

127. The compound of embodiment 113 or the pharmaceutically-acceptable salt thereof, the tautomer thereof, the pharmaceutically-acceptable salt of the tautomer, the stereoisomer thereof, or the mixture thereof, wherein the compound of Formula I has the Formula II:

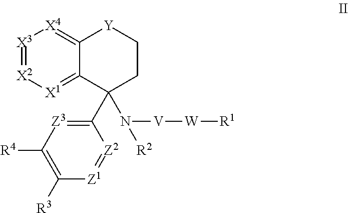

wherein:

Y is selected from —O— or —CH₂—.

128. The compound of embodiment 127 or the pharmaceutically-acceptable salt thereof, the tautomer thereof, the pharmaceutically-acceptable salt of the tautomer, the stereoisomer thereof, or the mixture thereof, wherein the compound of Formula II has the Formula IIA:

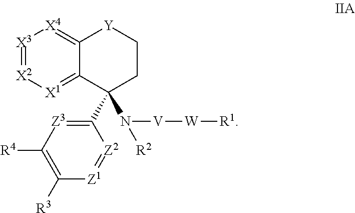

129. The compound of any one of embodiments 113-128 or the pharmaceutically-acceptable salt thereof, the tautomer thereof, the pharmaceutically-acceptable salt of the tautomer, the stereoisomer thereof, or the mixture thereof, wherein 0 or 1 of X¹, X², X³, and X⁴ are N.

130. The compound of embodiment 129 or the pharmaceutically-acceptable salt thereof, the tautomer thereof, the pharmaceutically-acceptable salt of the tautomer, the stereoisomer thereof, or the mixture thereof, wherein 1 of X¹, X², X³, and X⁴ are N.

131. The compound of embodiment 129 or the pharmaceutically-acceptable salt thereof, the tautomer thereof, the pharmaceutically-acceptable salt of the tautomer, the stereoisomer thereof, or the mixture thereof, wherein 0 of X¹, X², X³, and X⁴ are N.

132. The compound of embodiment 129 or the pharmaceutically-acceptable salt thereof, the tautomer thereof, the pharmaceutically-acceptable salt of the tautomer, the stereoisomer thereof, or the mixture thereof, wherein X¹ is N and X², X³, and X⁴ are all —CR⁵—.

133. The compound of embodiment 129 or the pharmaceutically-acceptable salt thereof, the tautomer thereof, the pharmaceutically-acceptable salt of the tautomer, the stereoisomer thereof, or the mixture thereof, wherein X² is N and X¹, X³, and X⁴ are all —CR⁵—.

134. The compound of embodiment 129 or the pharmaceutically-acceptable salt thereof, the tautomer thereof, the pharmaceutically-acceptable salt of the tautomer, the stereoisomer thereof, or the mixture thereof, wherein X³ is N and X¹, X², and X⁴ are all —CR⁵—.

135. The compound of embodiment 129 or the pharmaceutically-acceptable salt thereof, the tautomer thereof, the pharmaceutically-acceptable salt of the tautomer, the stereoisomer thereof, or the mixture thereof, wherein X⁴ is N and X¹, X², and X³ are all —CR⁵—.

136. The compound of any one of embodiments 113-128 or the pharmaceutically-acceptable salt thereof, the tautomer thereof, the pharmaceutically-acceptable salt of the tautomer, the stereoisomer thereof, or the mixture thereof, wherein 2 of X¹, X², X³, and X⁴ are N.

137. The compound of embodiment 136 or the pharmaceutically-acceptable salt thereof, the tautomer thereof, the pharmaceutically-acceptable salt of the tautomer, the stereoisomer thereof, or the mixture thereof, wherein X¹ and X⁴ are N and X² and X³ are —CR⁵—.

138. The compound of any one of embodiments 113-137 or the pharmaceutically-acceptable salt thereof, the tautomer thereof, the pharmaceutically-acceptable salt of the tautomer, the stereoisomer thereof, or the mixture thereof, wherein 0 or 1 of Z¹, Z², and Z³ are N.

139. The compound of embodiment 138 or the pharmaceutically-acceptable salt thereof, the tautomer thereof, the pharmaceutically-acceptable salt of the tautomer, the stereoisomer thereof, or the mixture thereof, wherein 0 of Z¹, Z², and Z³ are N.

140. The compound of embodiment 138 or the pharmaceutically-acceptable salt thereof, the tautomer thereof, the pharmaceutically-acceptable salt of the tautomer, the stereoisomer thereof, or the mixture thereof, wherein 1 of Z¹, Z², and Z³ are N.

141. The compound of embodiment 138 or the pharmaceutically-acceptable salt thereof, the tautomer thereof, the pharmaceutically-acceptable salt of the tautomer, the stereoisomer thereof, or the mixture thereof, wherein Z¹ is N and Z² and Z³ are —CR⁶—.

142. The compound of embodiment 138 or the pharmaceutically-acceptable salt thereof, the tautomer thereof, the pharmaceutically-acceptable salt of the tautomer, the stereoisomer thereof, or the mixture thereof, wherein Z² is N and Z¹ and Z³ are —CR⁶—.

143. The compound of embodiment 138 or the pharmaceutically-acceptable salt thereof, the tautomer thereof, the pharmaceutically-acceptable salt of the tautomer, the stereoisomer thereof, or the mixture thereof, wherein Z³ is N and Z¹ and Z² are —CR⁶—.

144. The compound of any one of embodiments 113-143 or the pharmaceutically-acceptable salt thereof, the tautomer thereof, the pharmaceutically-acceptable salt of the tautomer, the stereoisomer thereof, or the mixture thereof, wherein V is —C(═O)—.

145. The compound of any one of embodiments 113-143 or the pharmaceutically-acceptable salt thereof, the tautomer thereof, the pharmaceutically-acceptable salt of the tautomer, the stereoisomer thereof, or the mixture thereof, wherein V is —S(═O)₂—.

146. The compound of any one of embodiments 113-145 or the pharmaceutically-acceptable salt thereof, the tautomer thereof, the pharmaceutically-acceptable salt of the tautomer, the stereoisomer thereof, or the mixture thereof, wherein W is absent.

147. The compound of any one of embodiments 113-145 or the pharmaceutically-acceptable salt thereof, the tautomer thereof, the pharmaceutically-acceptable salt of the tautomer, the stereoisomer thereof, or the mixture thereof, wherein W is —NH—.

148. The compound of any one of embodiments 113-145 or the pharmaceutically-acceptable salt thereof, the tautomer thereof, the pharmaceutically-acceptable salt of the tautomer, the stereoisomer thereof, or the mixture thereof, wherein W is —O—.

149. The compound of any one of embodiments 113-148 or the pharmaceutically-acceptable salt thereof, the tautomer thereof, the pharmaceutically-acceptable salt of the tautomer, the stereoisomer thereof, or the mixture thereof, wherein R² is H.

150. The compound of embodiment 113 or the pharmaceutically-acceptable salt thereof, the tautomer thereof, the pharmaceutically-acceptable salt of the tautomer, the stereoisomer thereof, or the mixture thereof, wherein the compound of Formula I has the Formula III

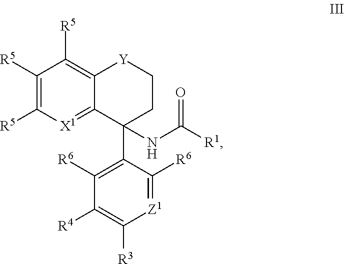

wherein Y is —O— or —CH₂—. In some such embodiments, the compound has the Formula III′

151. The compound of embodiment 150 or the pharmaceutically-acceptable salt thereof, the tautomer thereof, the pharmaceutically-acceptable salt of the tautomer, the stereoisomer thereof, or the mixture thereof, wherein the compound of Formula III has the Formula IIIA, IIIB, IIIC, or IIID

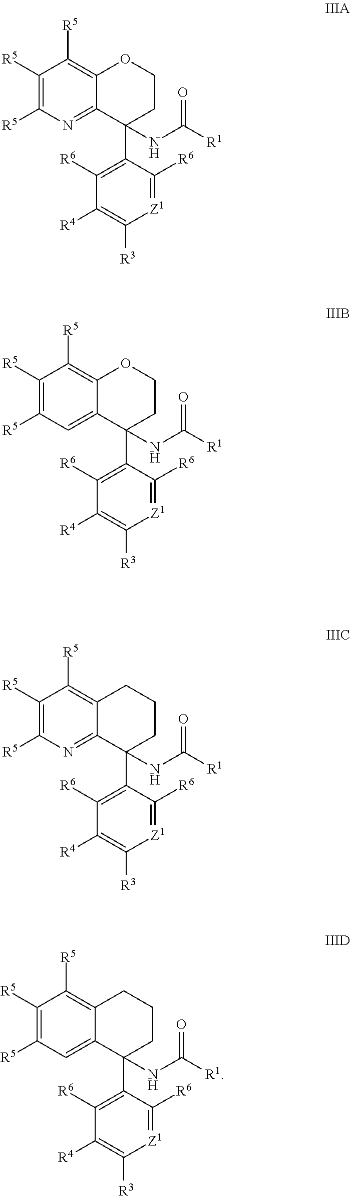

152. The compound of embodiment 151 or the pharmaceutically-acceptable salt thereof, the tautomer thereof, the pharmaceutically-acceptable salt of the tautomer, the stereoisomer thereof, or the mixture thereof, wherein the compound of Formula III has the Formula IIIA. In some such embodiments, the compound has the Formula IIIA′.

153. The compound of embodiment 151 or the pharmaceutically-acceptable salt thereof, the tautomer thereof, the pharmaceutically-acceptable salt of the tautomer, the stereoisomer thereof, or the mixture thereof, wherein the compound of Formula III has the Formula IIIB. In some such embodiments, the compound has the Formula IIIB′

154. The compound of embodiment 151 or the pharmaceutically-acceptable salt thereof, the tautomer thereof, the pharmaceutically-acceptable salt of the tautomer, the stereoisomer thereof, or the mixture thereof, wherein the compound of Formula III has the Formula IIIC. In some such embodiments, the compound has the Formula IIIC′.

155. The compound of embodiment 151 or the pharmaceutically-acceptable salt thereof, the tautomer thereof, the pharmaceutically-acceptable salt of the tautomer, the stereoisomer thereof, or the mixture thereof, wherein the compound of Formula III has the Formula IIID. In some such embodiments, the compound has the Formula IIID′

156. The compound of embodiment 113 or the pharmaceutically-acceptable salt thereof, the tautomer thereof, the pharmaceutically-acceptable salt of the tautomer, the stereoisomer thereof, or the mixture thereof, wherein the compound of Formula I has the Formula IV

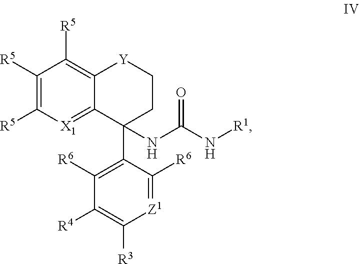

wherein Y is —O— or —CH₂—. In some such embodiments, the compound has the Formula IV′.

157. The compound of embodiment 156 or the pharmaceutically-acceptable salt thereof, the tautomer thereof, the pharmaceutically-acceptable salt of the tautomer, the stereoisomer thereof, or the mixture thereof, wherein the compound of Formula IV has the Formula IVA, IVB, IVC, or IVD

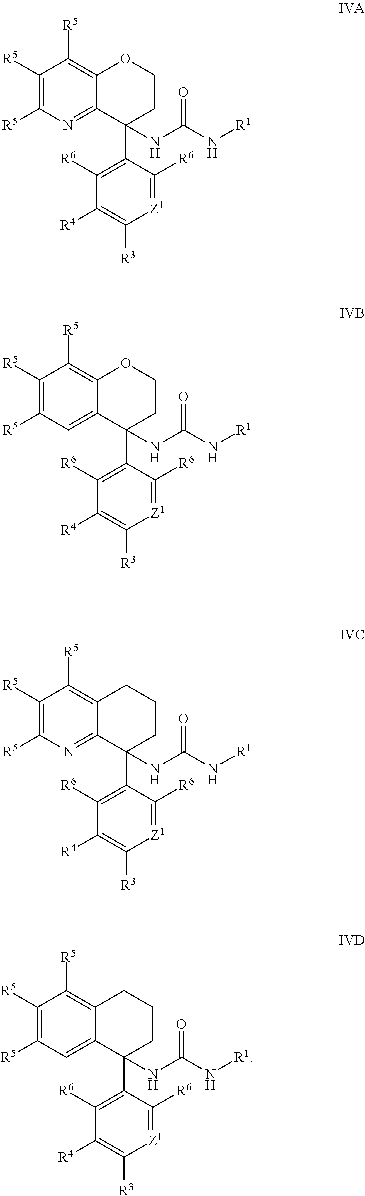

158. The compound of embodiment 157 or the pharmaceutically-acceptable salt thereof, the tautomer thereof, the pharmaceutically-acceptable salt of the tautomer, the stereoisomer thereof, or the mixture thereof, wherein the compound of Formula IV has the Formula IVA. In some such embodiments, the compound has the Formula IVA′.

159. The compound of embodiment 157 or the pharmaceutically-acceptable salt thereof, the tautomer thereof, the pharmaceutically-acceptable salt of the tautomer, the stereoisomer thereof, or the mixture thereof, wherein the compound of Formula IV has the Formula IVB. In some such embodiments, the compound has the Formula IVB′.

160. The compound of embodiment 157 or the pharmaceutically-acceptable salt thereof, the tautomer thereof, the pharmaceutically-acceptable salt of the tautomer, the stereoisomer thereof, or the mixture thereof, wherein the compound of Formula IV has the Formula IVC. In some such embodiments, the compound has the Formula IVC′.

161. The compound of embodiment 157 or the pharmaceutically-acceptable salt thereof, the tautomer thereof, the pharmaceutically-acceptable salt of the tautomer, the stereoisomer thereof, or the mixture thereof, wherein the compound of Formula IV has the Formula IVD. In some such embodiments, the compound has the Formula IVD′.

162. The compound of any one of embodiments 113-161 or the pharmaceutically-acceptable salt thereof, the tautomer thereof, the pharmaceutically-acceptable salt of the tautomer, the stereoisomer thereof, or the mixture thereof, wherein R³ is selected from H, C₁₋₈alk, C₁₋₈alkOH, C₁₋₄haloalk, halo, or —OR^{a}.

163. The compound of any one of embodiments 113-161 or the pharmaceutically-acceptable salt thereof, the tautomer thereof, the pharmaceutically-acceptable salt of the tautomer, the stereoisomer thereof, or the mixture thereof, wherein R³ is selected from —H, —CH₃, —F, —Cl, —CF₃, or —OCF₃.

164. The compound of embodiment 163 or the pharmaceutically-acceptable salt thereof, the tautomer thereof, the pharmaceutically-acceptable salt of the tautomer, the stereoisomer thereof, or the mixture thereof, wherein R³ is selected from —CH₃, —F, —Cl, —CF₃, or —OCF₃.

165. The compound of embodiment 163 or the pharmaceutically-acceptable salt thereof, the tautomer thereof, the pharmaceutically-acceptable salt of the tautomer, the stereoisomer thereof, or the mixture thereof, wherein R³ is selected from —CF₃ or —OCF₃.

166. The compound of any one of embodiments 113-161 or the pharmaceutically-acceptable salt thereof, the tautomer thereof, the pharmaceutically-acceptable salt of the tautomer, the stereoisomer thereof, or the mixture thereof, wherein R³ is R^{b} and R^{b} is a phenyl substituted by 0, 1, 2 or 3 substituents selected from halo, cyano, oxo, C₁₋₄alk, C₁₋₄alkOH, C₁₋₃haloalk, —OC₁₋₄alk, —OH, —NH₂, —OC₁₋₄alk, —OC₁₋₄haloalk, —S(═O)₂C₁₋₄alk, —NHC(═O)—C₁₋₄alk, —C(═O)NH₂, —C(═O)NHC₁₋₄alk, —C(═O)N(C₁₋₄alk)₂, —NHC₁₋₄alk, and —N(C₁₋₄alk)C₁₋₄alk, or a saturated, partially-saturated, or unsaturated 5 or 6-membered monocyclic ring containing 1 or 2 heteroatoms selected from N, O, and S.

167. The compound of any one of embodiments 113-161 or the pharmaceutically-acceptable salt thereof, the tautomer thereof, the pharmaceutically-acceptable salt of the tautomer, the stereoisomer thereof, or the mixture thereof, wherein R³ is R^{b} and R^{b} is an unsaturated 5 or 6-membered monocyclic ring containing 1, 2, or 3 heteroatoms selected from N, O, and S, wherein the 5 or 6-membered monocyclic ring is substituted by 0. 1, 2, or 3 substituents selected from halo, cyano, oxo, C₁₋₄alk, C₁₋₄alkOH, C₁₋₃haloalk, —OCl₁₋₄alk, —OH, —NH₂, —OC₁₋₄alk, —OC₁₋₄haloalk, —S(═O)₂C₁₋₄alk, —NHC(═O)—C₁₋₄alk, —C(═O)NH₂, —C(═O)NHC₁₋₄alk, —C(═O)N(C₁₋₄alk)₂, —NHC₁₋₄alk, and —N(C₁₋₄alk)C₁₋₄alk, or a saturated, partially-saturated, or unsaturated 5 or 6-membered monocyclic ring containing 1 or 2 heteroatoms selected from N, O, and S.

168. The compound of any one of embodiments 113-161 or the pharmaceutically-acceptable salt thereof, the tautomer thereof, the pharmaceutically-acceptable salt of the tautomer, the stereoisomer thereof, or the mixture thereof, wherein R³ is R^{b} and R^{b} is a pyridyl, pyrazinyl, pyrimidinyl, isoxazolyl, or furanyl substituted by 0. 1, 2, or 3 substituents selected from halo, cyano, oxo, C₁₋₄alk, C₁₋₄alkOH, C₁₋₃haloalk, —OC₁₋₄alk, —OH, —NH₂, —OC₁₋₄alk, —OC₁₋₄haloalk, —S(═O)₂C₁₋₄alk, —NHC(═O)—C₁₋₄alk, —C(═O)NH₂, —C(═O)NHC₁₋₄alk, —C(═O)N(C₁₋₄alk₂, —NHC₁₋₄alk, and —N(C₁₋₄alk)C₁₋₄alk, or a saturated, partially-saturated, or unsaturated 5 or 6-membered monocyclic ring containing 1 or 2 heteroatoms selected from N, O, and S.

169. The compound of any one of embodiments 113-168 or the pharmaceutically-acceptable salt thereof, the tautomer thereof, the pharmaceutically-acceptable salt of the tautomer, the stereoisomer thereof, or the mixture thereof, wherein R⁴ is H.

170. The compound of any one of embodiments 113-168 or the pharmaceutically-acceptable salt thereof, the tautomer thereof, the pharmaceutically-acceptable salt of the tautomer, the stereoisomer thereof, or the mixture thereof, wherein R⁴ is selected from F, Cl, C₁₋₆alk, —OC_{1-b}alk, —OC₁₋₃haloalk, or —C₁₋₃haloalk.

171. The compound of embodiment 170 or the pharmaceutically-acceptable salt thereof, the tautomer thereof, the pharmaceutically-acceptable salt of the tautomer, the stereoisomer thereof, or the mixture thereof, wherein R⁴ is selected from —F, —Cl, or —OCF₃.

172. The compound of embodiment 171 or the pharmaceutically-acceptable salt thereof, the tautomer thereof, the pharmaceutically-acceptable salt of the tautomer, the stereoisomer thereof, or the mixture thereof, wherein R⁴ is —F.

173. The compound of any one of embodiments 113-161 or the pharmaceutically-acceptable salt thereof, the tautomer thereof, the pharmaceutically-acceptable salt of the tautomer, the stereoisomer thereof, or the mixture thereof, wherein —R³ is —OCF₃ and R⁴ is —F.

174. The compound of any one of embodiments 113-161 or the pharmaceutically-acceptable salt thereof, the tautomer thereof, the pharmaceutically-acceptable salt of the tautomer, the stereoisomer thereof, or the mixture thereof, wherein R³ and R⁴ together form a four-atom unsaturated bridge containing 0 or 1 N atoms, wherein the bridge is substituted by 0, 1 or 2 R⁵ substituents.

175. The compound of any one of embodiments 113-174 or the pharmaceutically-acceptable salt thereof, the tautomer thereof, the pharmaceutically-acceptable salt of the tautomer, the stereoisomer thereof, or the mixture thereof, wherein each instance of R⁵ is H.

176. The compound of any one of embodiments 113-174 or the pharmaceutically-acceptable salt thereof, the tautomer thereof, the pharmaceutically-acceptable salt of the tautomer, the stereoisomer thereof, or the mixture thereof, wherein in at least one instance, R⁵ is selected from C₁₋₈alk, halo, or —OR^{a}.

177. The compound of any one of embodiments 113-174 or the pharmaceutically-acceptable salt thereof, the tautomer thereof, the pharmaceutically-acceptable salt of the tautomer, the stereoisomer thereof, or the mixture thereof, wherein in at least one instance, R⁵ is selected from —CH₃, —Cl, —F, or —OMe.

178. The compound of any one of embodiments 113-177 or the pharmaceutically-acceptable salt thereof, the tautomer thereof, the pharmaceutically-acceptable salt of the tautomer, the stereoisomer thereof, or the mixture thereof, wherein each instance of R⁶ is H.

179. The compound of any one of embodiments 113-177 or the pharmaceutically-acceptable salt thereof, the tautomer thereof, the pharmaceutically-acceptable salt of the tautomer, the stereoisomer thereof, or the mixture thereof, wherein at least one instance of R⁶ is halo or C₁₋₆alk.

180. The compound of any one of embodiments 113-177 or the pharmaceutically-acceptable salt thereof, the tautomer thereof, the pharmaceutically-acceptable salt of the tautomer, the stereoisomer thereof, or the mixture thereof, wherein at least one instance of R⁶ is —F, —Cl, or —CH₃.

181. The compound of any one of embodiments 113-180 or the pharmaceutically-acceptable salt thereof, the tautomer thereof, the pharmaceutically-acceptable salt of the tautomer, the stereoisomer thereof, or the mixture thereof, wherein R¹ is the saturated, partially-saturated or unsaturated 3-, 4-, 5-, 6- or 7-membered monocyclic or 7-, 8-, 9-, 10- or 11-membered bicyclic ring and the monocyclic or bicyclic ring is substituted by 0, 1, 2, or 3 substituents, wherein the substituents are selected from F, Cl, Br, I, oxo, cyano, —CH₃, —CH₂CH₃, —CH₂CH₂CH₃, —C(H)(CH₃)₂, —CH₂C(H)(CH₃)₂, —CH₂C(H)═CF₁₂, —CH₂CO₂H, —CH₂CF₃, —C(OH)(CH₃)₂, —SO₂N(H)CH₃, —N(H)SO₂CH₃, —OCH₃, —OCF₃, —OH, —OCH₂CO₂H, —CH₂OH, —CH₂CH₂OH, —CH₂C(H)(CH₃)OH, —CO₂H, —CO₂CH₃, —CO₂CH₂CH₃, —CO₂C(CH₃)₃, —CO₂NH₂, —CO₂N(H)CH₃, —SO₂CH₃, —OC(═O)CH₃, —NH₂, —NHC(═O)CH₃, —N(CH₃)₂, —N(H)CH₂CH₃, —CF₃, —CHF₂, —CH₂C(H)(CF₃)OH, —CH₂C(CH₃)₂OH, —CH₂-phenyl, —C(═O)-phenyl, tetrazolyl, oxadiazolonyl, pyridyl, oxetanyl,

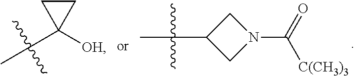 [SMILES: *C1(O)CC1 ; "or" ; *C1CN(C(=O)C(C)(C)C)C1 ; "."]

182. The compound of any one of embodiments 113-180 or the pharmaceutically-acceptable salt thereof, the tautomer thereof, the pharmaceutically-acceptable salt of the tautomer, the stereoisomer thereof, or the mixture thereof, wherein R¹ is a phenyl, pyridyl, pyridinonyl, piperidinonyl, pyridazinonyl, pyrazinonyl, pyridazinyl, pyrimidinyl, pyrazinyl, tetradyrofuranyl, tetrahydropyranyl, thiazolyl, isothiazolyl, furanyl, thiophenyl, pyrazolyl, isoxazolyl, triazolyl, oxazolyl, imidazolyl, pyrrolidinonyl, piperidinyl, cyclohexyl, cyclohexanonyl, quinolinyl, isoquinolinyl, naphthyridinyl, pyrrolopyridinyl, pyrrolopyrimidinyl, benzothiophenyl, pyrazolopyrimidinyl, triazolopyrimidinyl, indazolyl, tetrahydroindazolyl, tetrahydrocyclopentapyrazolyl, dihydropyrazolooxazinyl, indolinonyl, isoindolinonyl, benzooxazolonyl, oxazolopyridinonyl, benzoimidazolonyl, isoindolindionyl, tetrahydroquinolinyl, dihydroquinolinonyl, benzooxazinonyl, dihydrobenzooxazinonyl, dihydroindenonyl, benzothiazolyl, benzimidazolyl, imidazopyridinyl, tetrazolopyridinyl, quinolinonyl, quinoxalinyl, indolyl, or quinoxalindionyl substituted by 0, 1, 2, or 3 substituents.

183. The compound of any one of embodiments 113-180 or the pharmaceutically-acceptable salt thereof, the tautomer thereof, the pharmaceutically-acceptable salt of the tautomer, the stereoisomer thereof, or the mixture thereof, wherein R¹ is a phenyl, pyridyl, pyridinonyl, pyrazinonyl, pyridazinyl, pyrimidinyl, tetrahydropyranyl, thiazolyl, isothiazolyl, imidazolyl, piperidinyl, quinolinyl, isoquinolinyl, indazolyl, indolinonyl, isoindolinonyl, benzooxazolonyl, dihydroquinolinonyl, imidazopyridinyl, quinolinonyl, indolyl substituted by 0, 1, 2, or 3 substituents.

184. The compound of embodiment 183 or the pharmaceutically-acceptable salt thereof, the tautomer thereof, the pharmaceutically-acceptable salt of the tautomer, the stereoisomer thereof, or the mixture thereof, wherein R¹ is a phenyl substituted by 0, or 1 substituent.

185. The compound of embodiment 183 or the pharmaceutically-acceptable salt thereof, the tautomer thereof, the pharmaceutically-acceptable salt of the tautomer, the stereoisomer thereof, or the mixture thereof, wherein R¹ is a pyridinonyl substituted by 0, or 1 substituent.

186. The compound of embodiment 183 or the pharmaceutically-acceptable salt thereof, the tautomer thereof, the pharmaceutically-acceptable salt of the tautomer, the stereoisomer thereof, or the mixture thereof, wherein R¹ is a pyridyl substituted by 0, or 1 substituent.

187. The compound of embodiment 183 or the pharmaceutically-acceptable salt thereof, the tautomer thereof, the pharmaceutically-acceptable salt of the tautomer, the stereoisomer thereof, or the mixture thereof, wherein R¹ is a benzooxazolonyl substituted by 0, or 1 substituent.

188. The compound of embodiment 183 or the pharmaceutically-acceptable salt thereof, the tautomer thereof, the pharmaceutically-acceptable salt of the tautomer, the stereoisomer thereof, or the mixture thereof, wherein R¹ is a quinolinyl substituted by 0, or 1 substituent.

189. The compound of any one of embodiments 113-180 or the pharmaceutically-acceptable salt thereof, the tautomer thereof, the pharmaceutically-acceptable salt of the tautomer, the stereoisomer thereof, or the mixture thereof, wherein R¹ is a group of formula

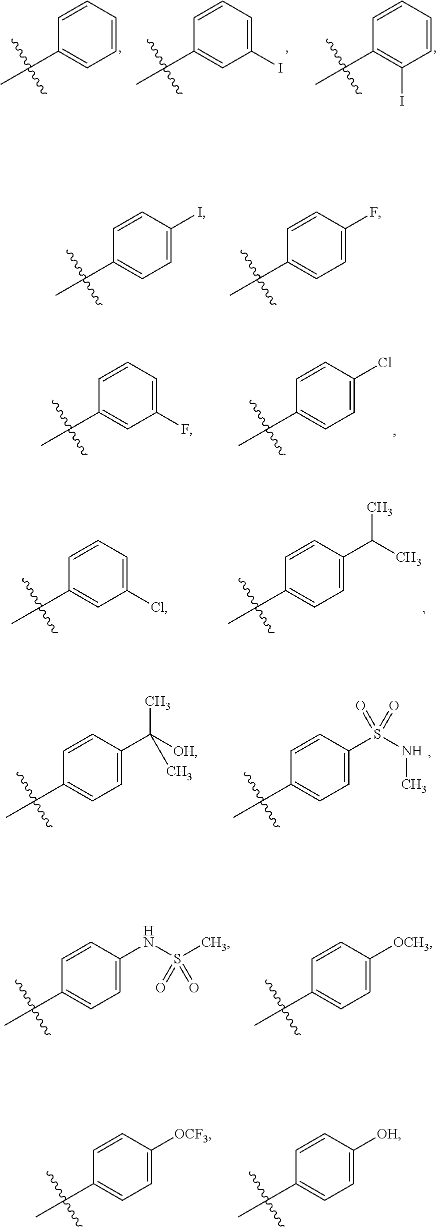

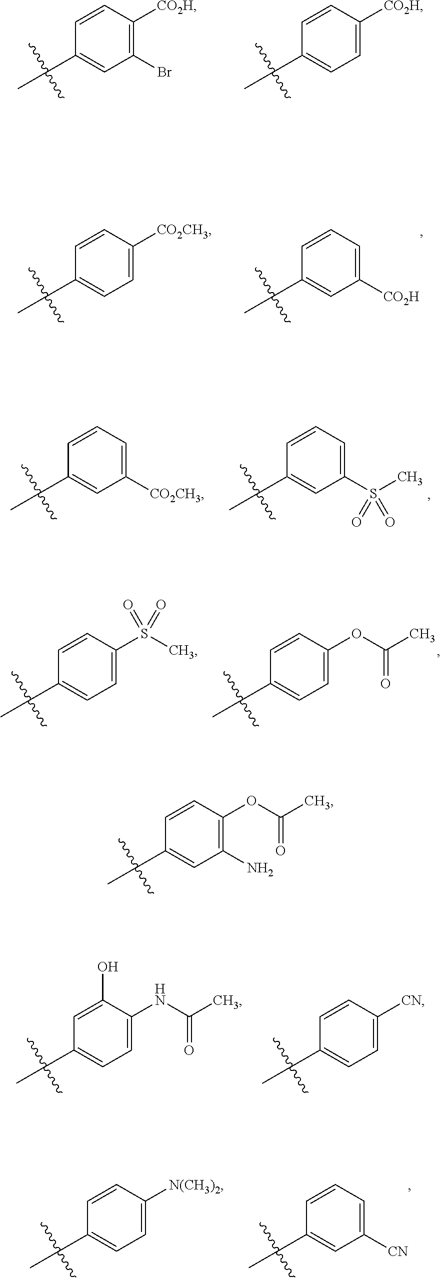

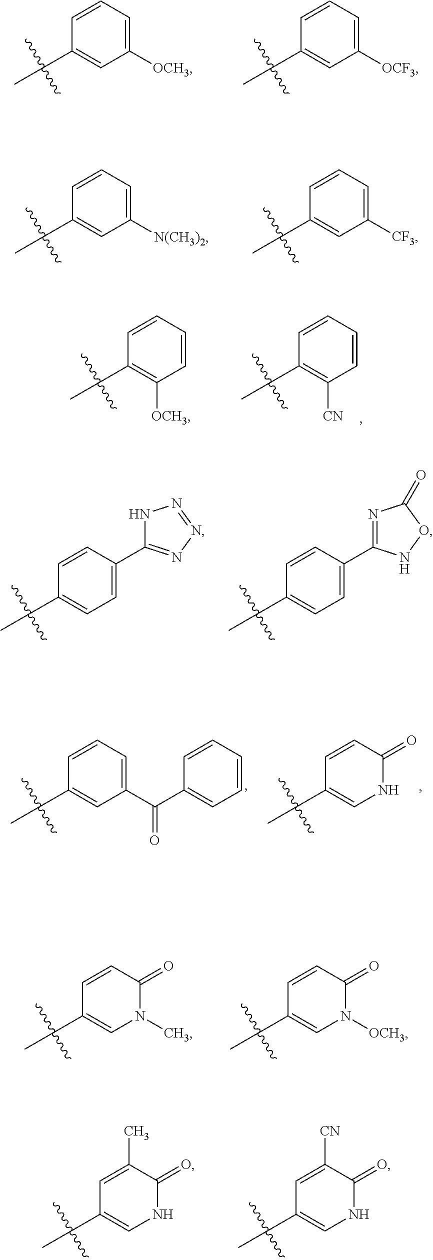

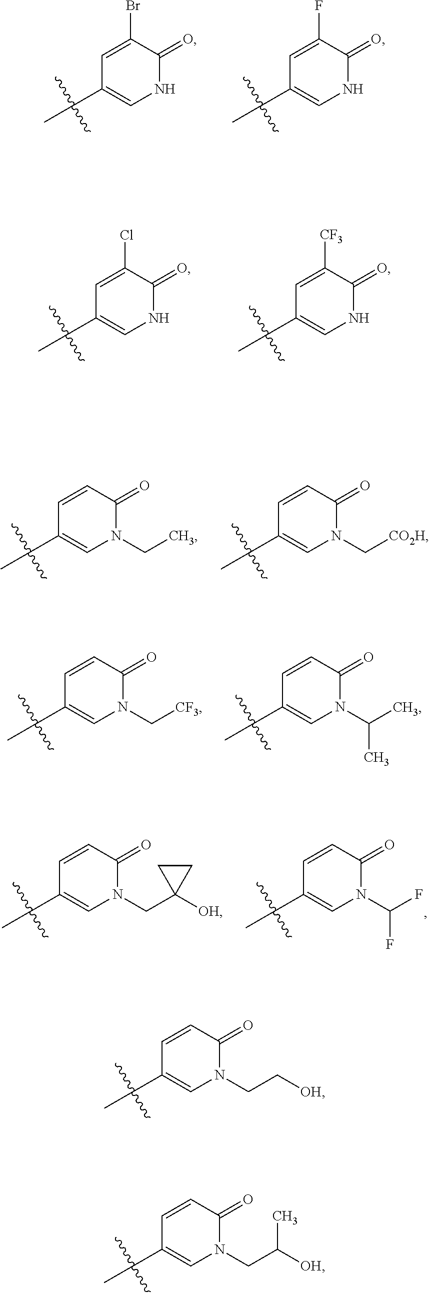

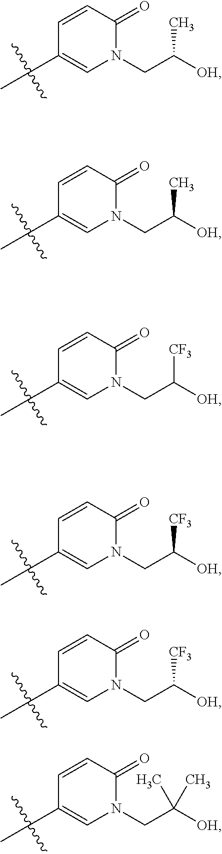 [SMILES: *c1ccc(=O)n(C[C@H](C)O)c1 ; *c1ccc(=O)n(C[C@@H](C)O)c1 ; *c1ccc(=O)n(CC(O)C(F)(F)F)c1 ; *c1ccc(=O)n(C[C@H](O)C(F)(F)F)c1 ; *c1ccc(=O)n(C[C@@H](O)C(F)(F)F)c1 ; *c1ccc(=O)n(CC(C)(C)O)c1]

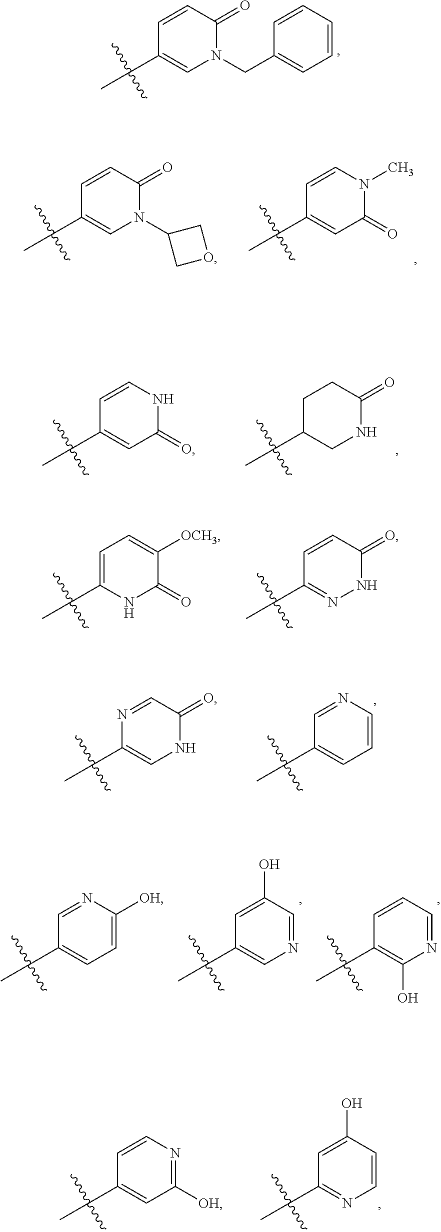

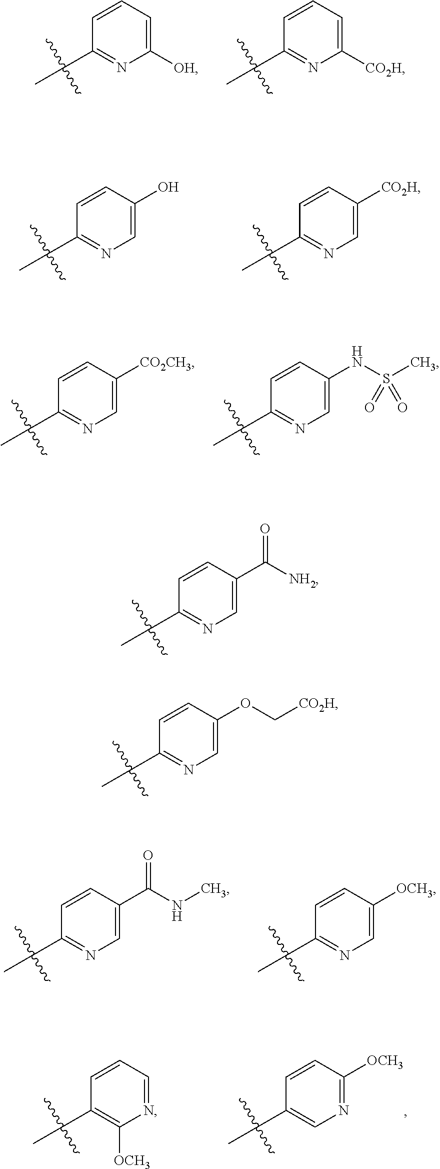

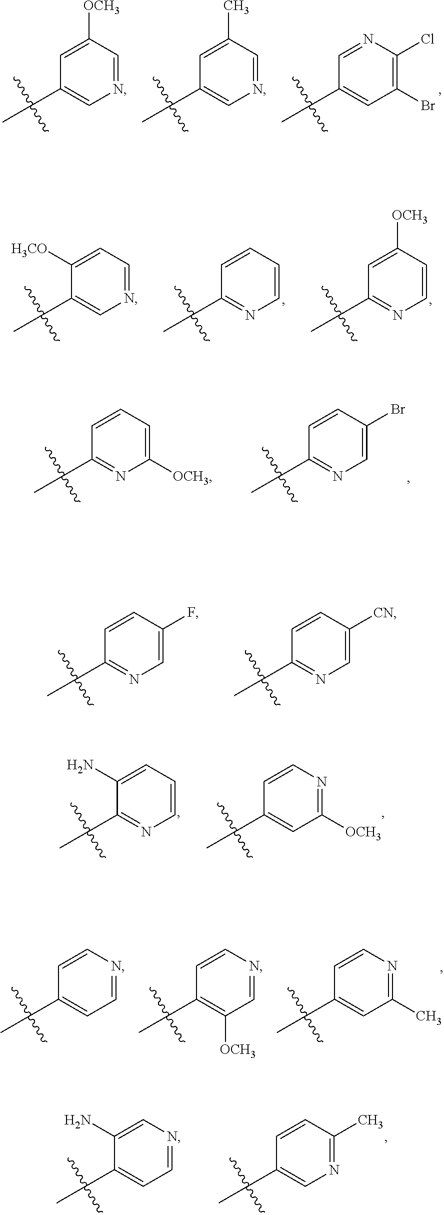

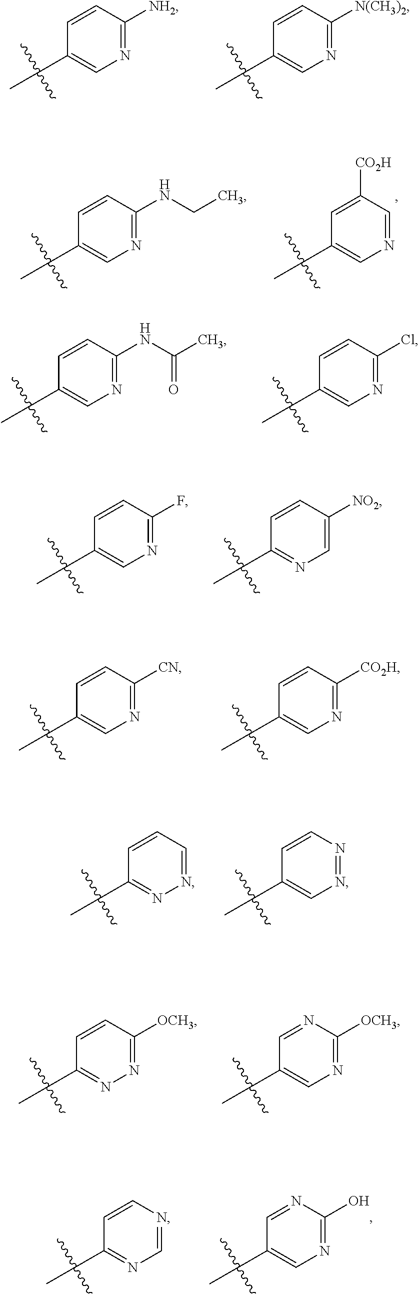

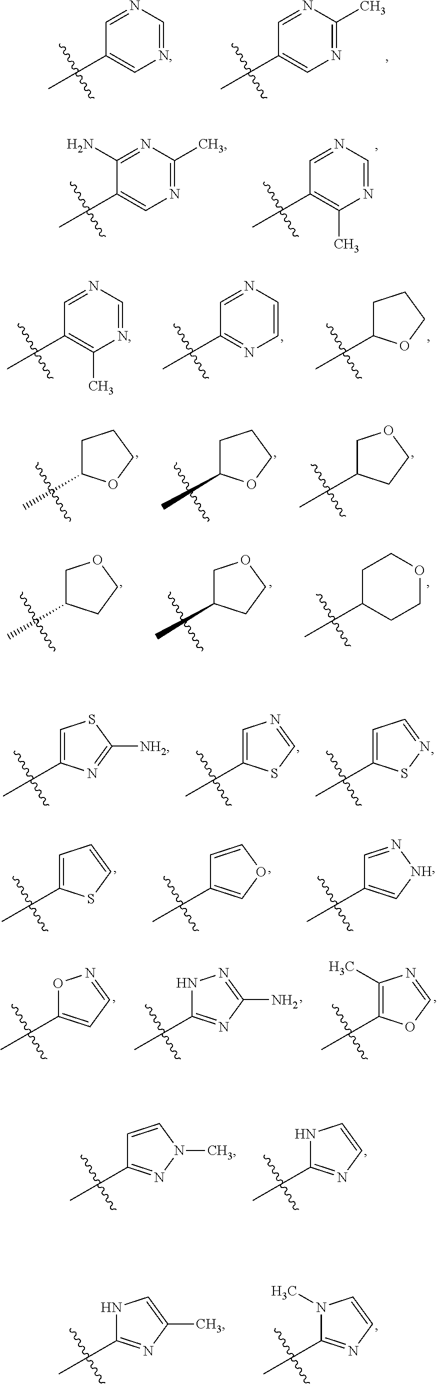

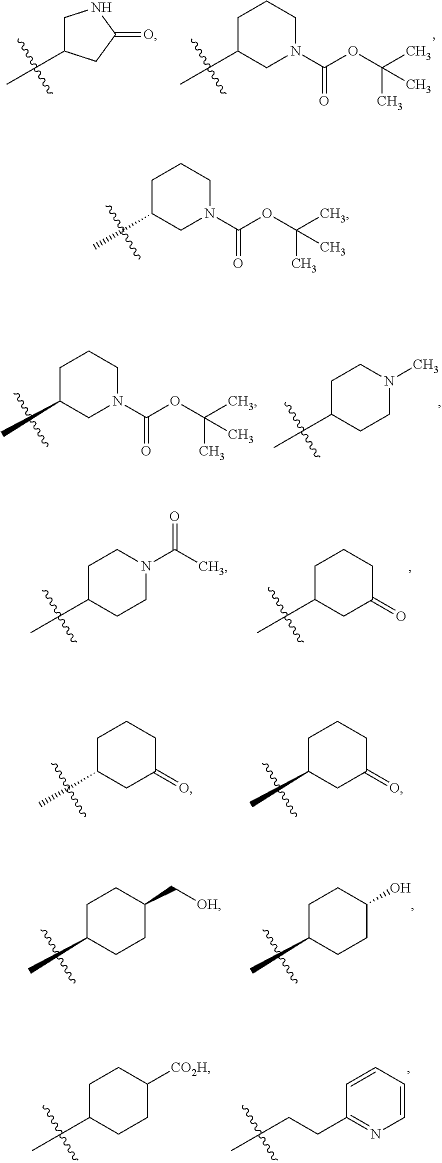

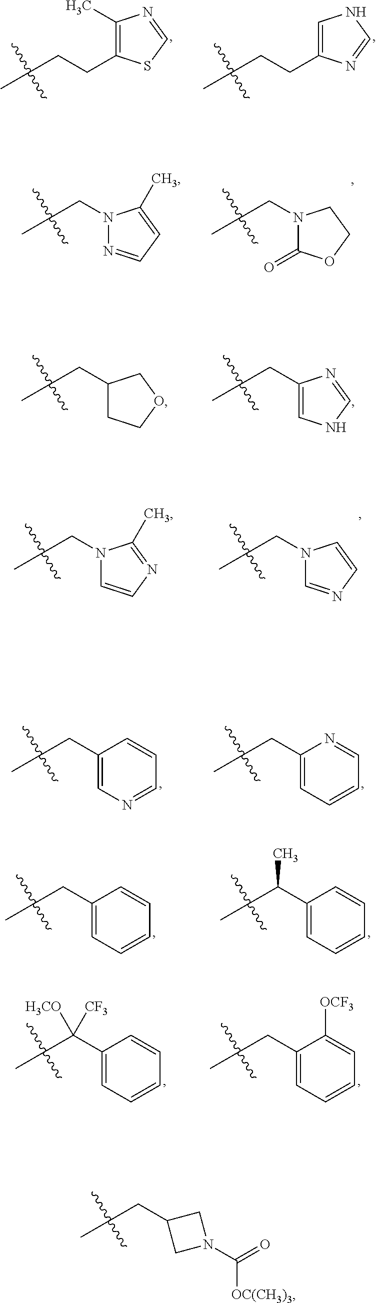

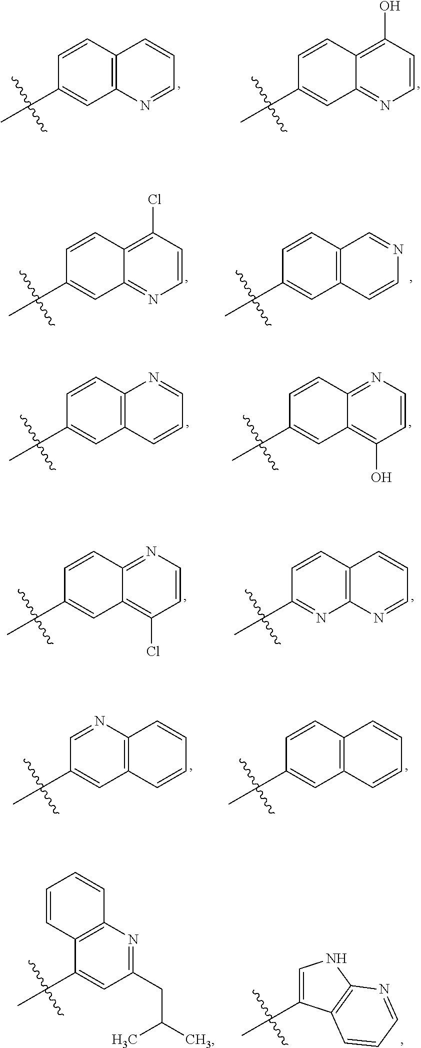

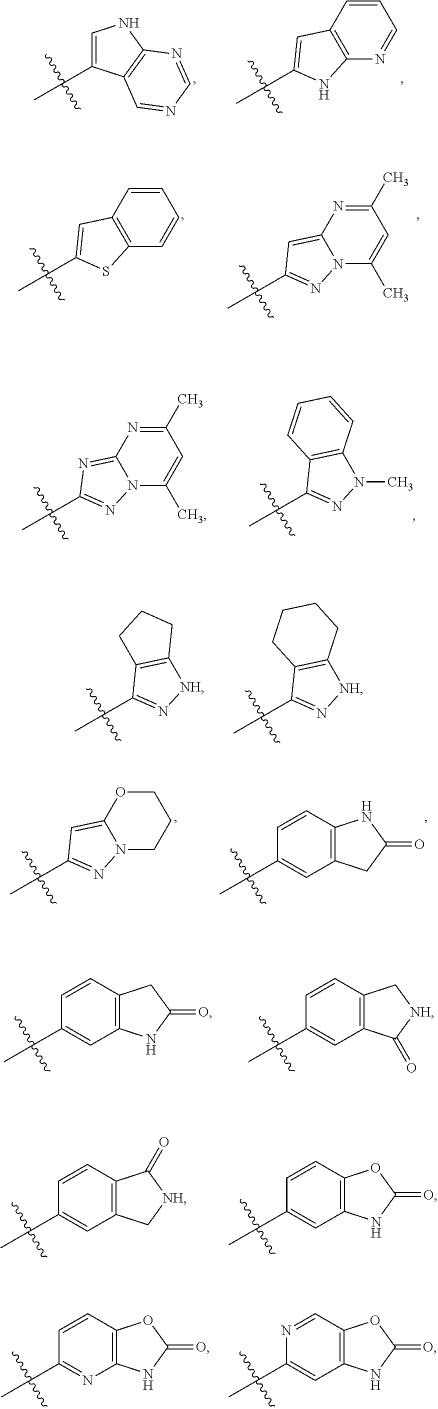

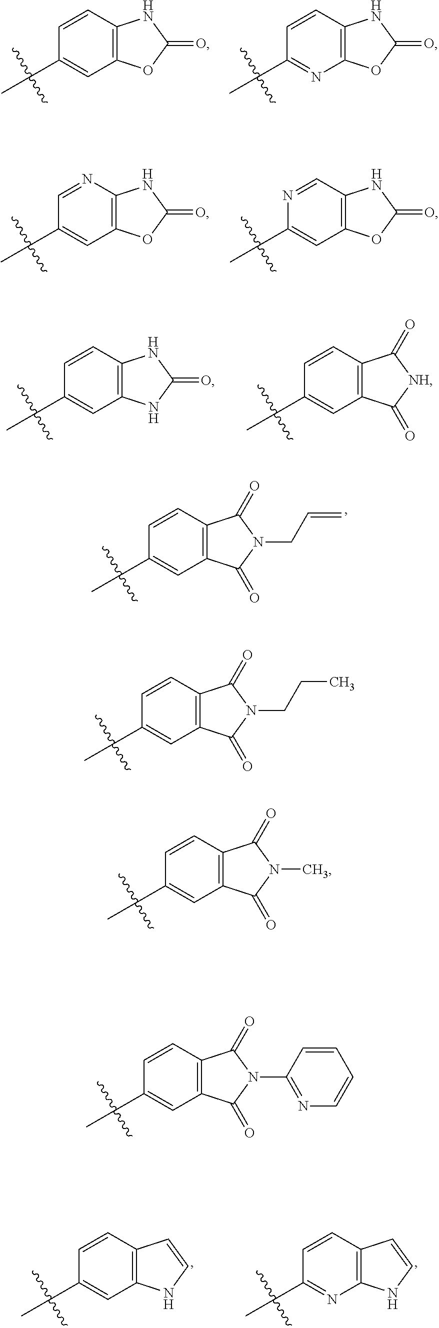

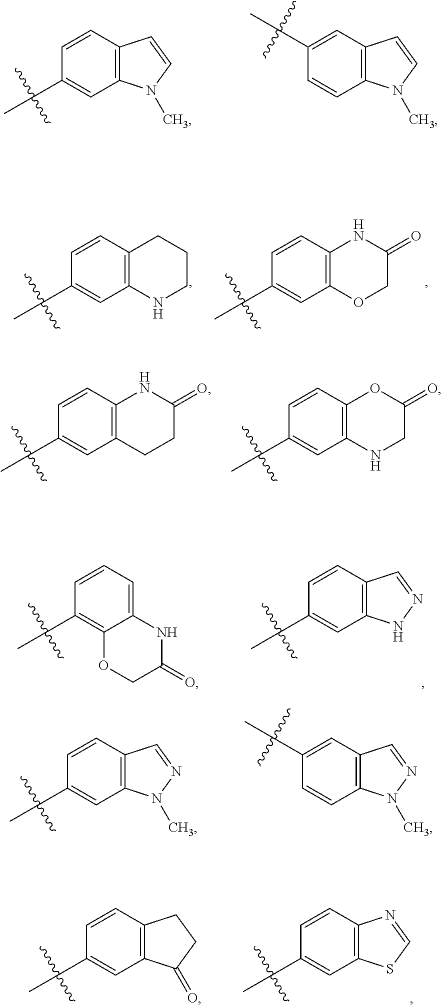

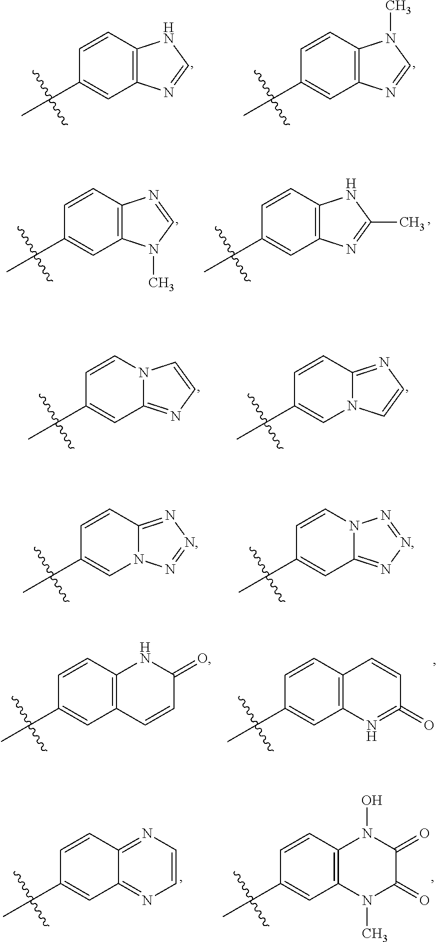

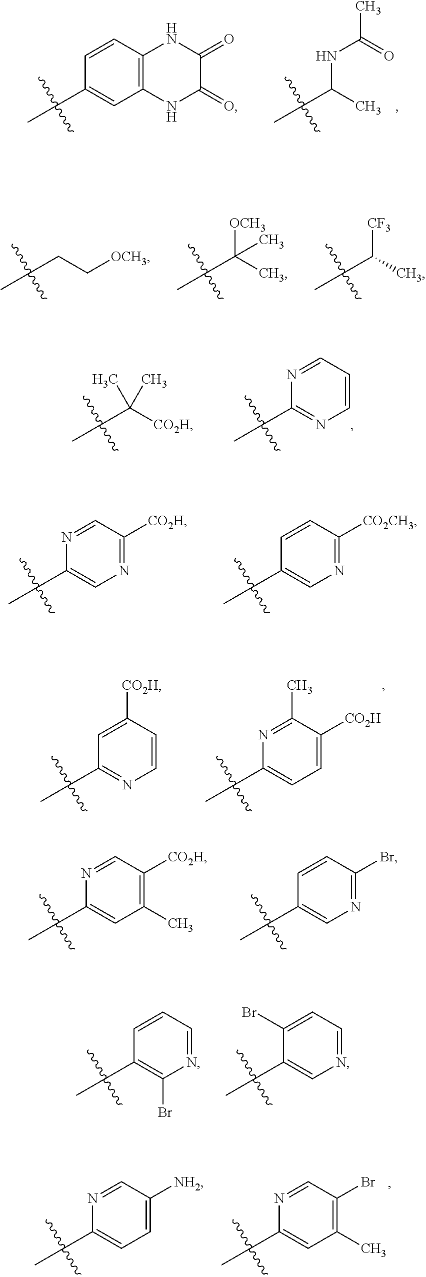

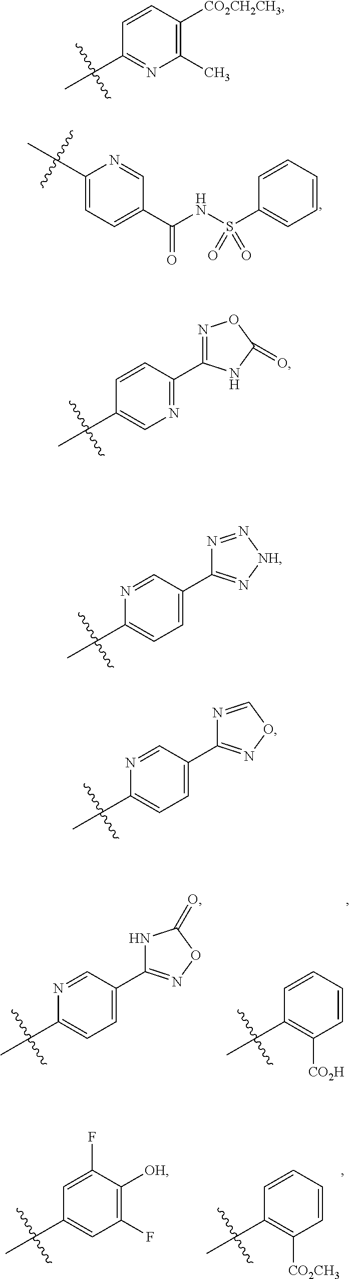

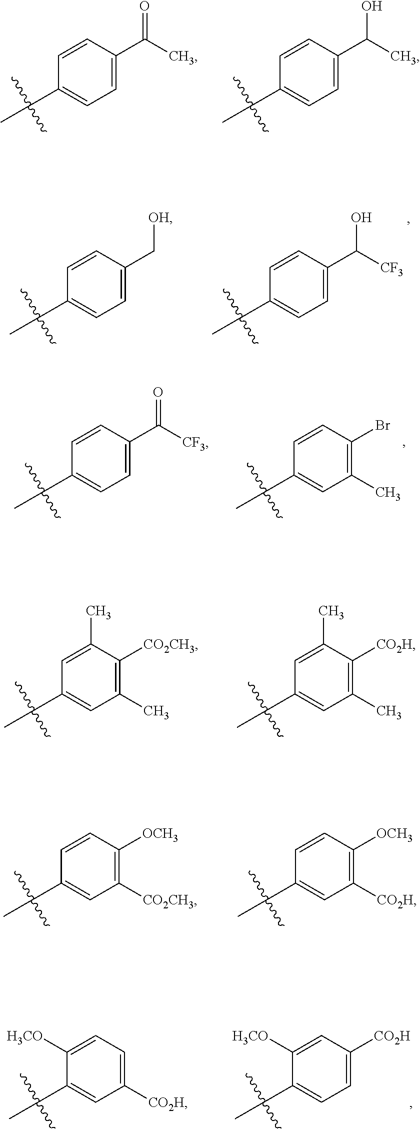

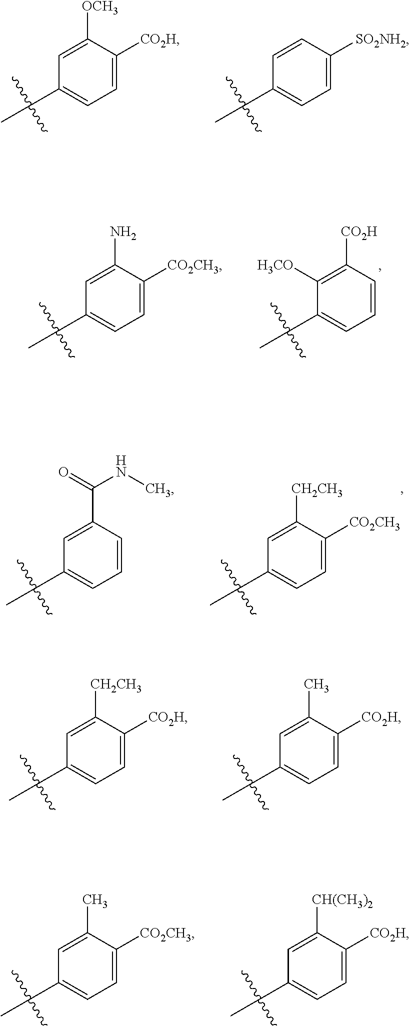

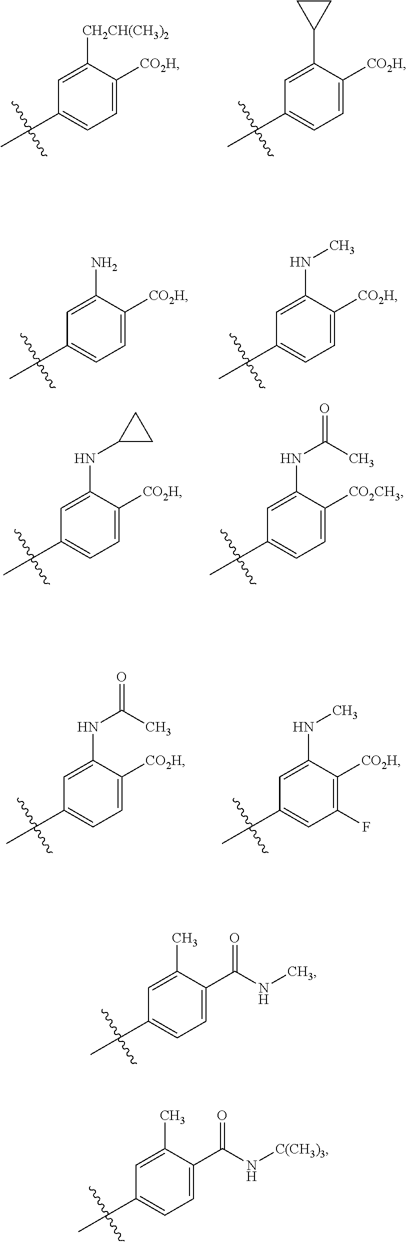

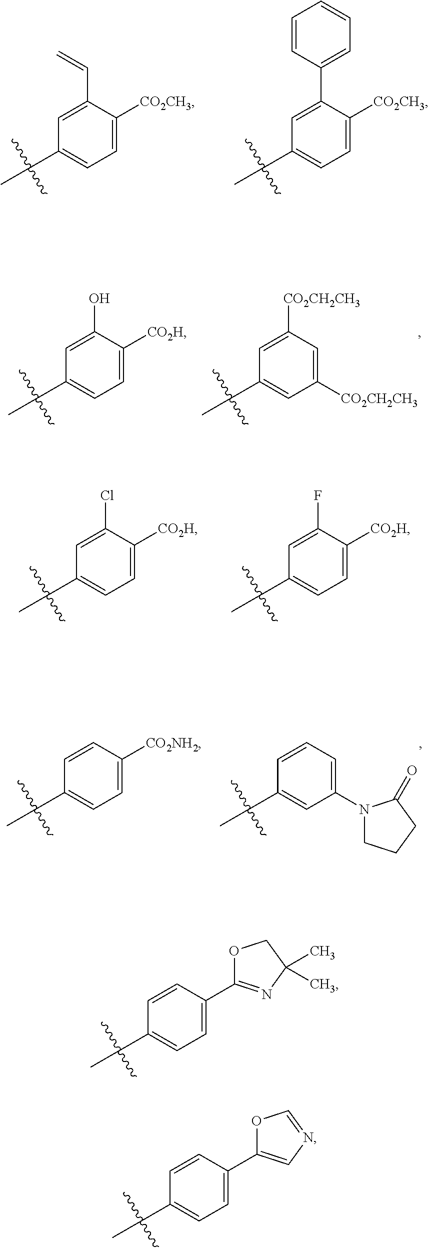

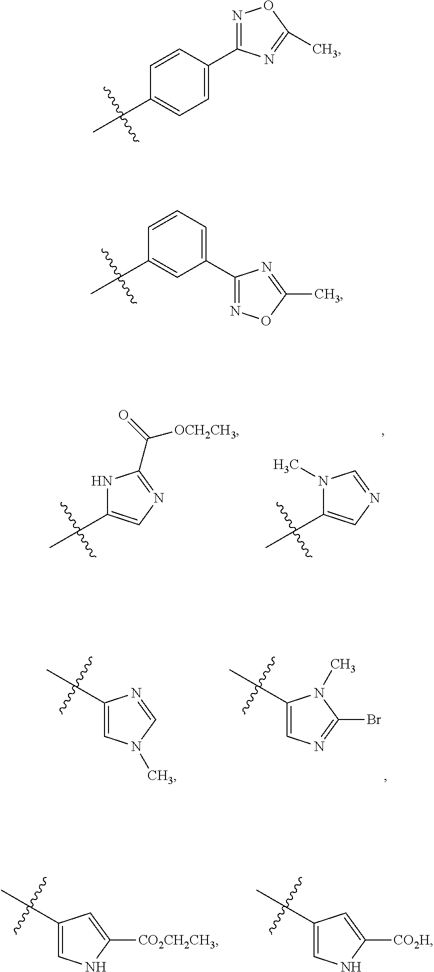

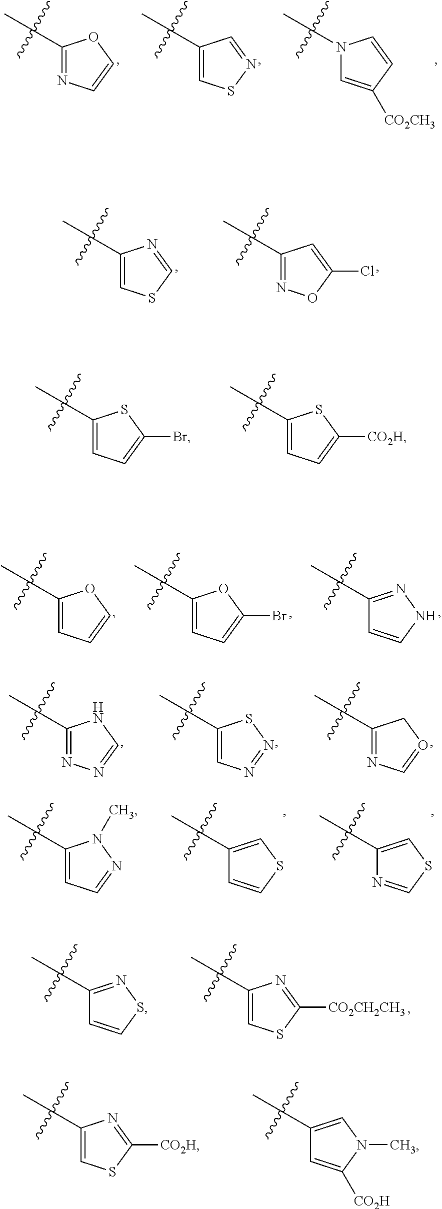

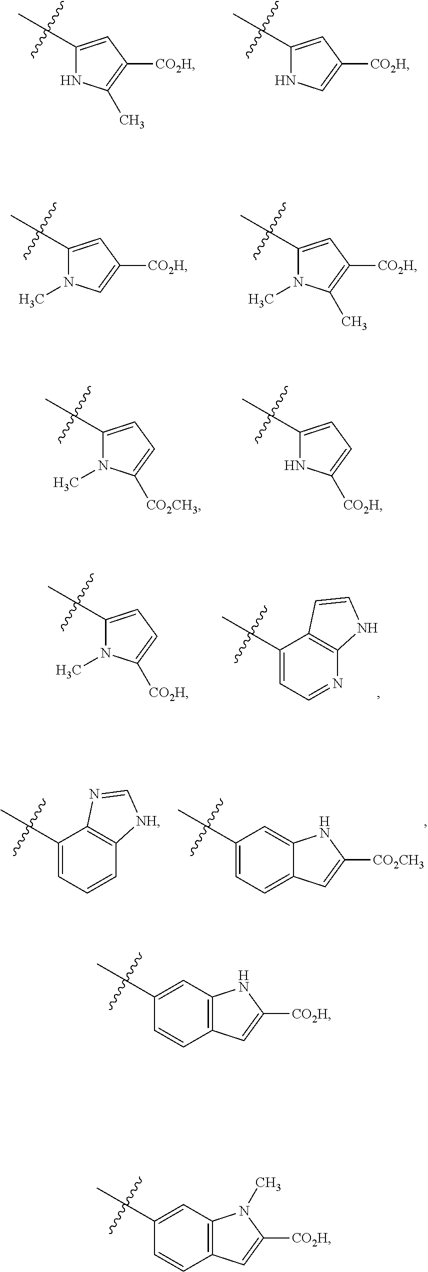

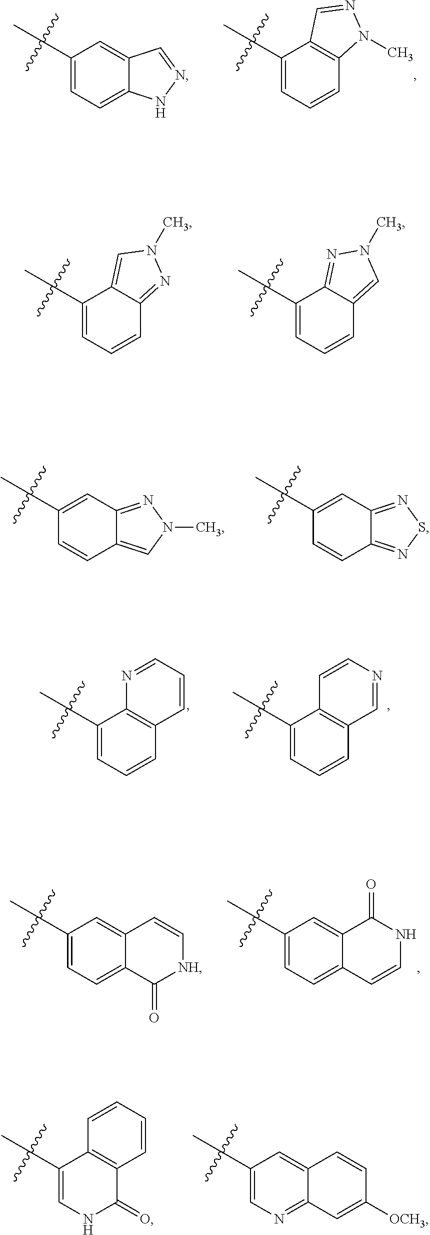

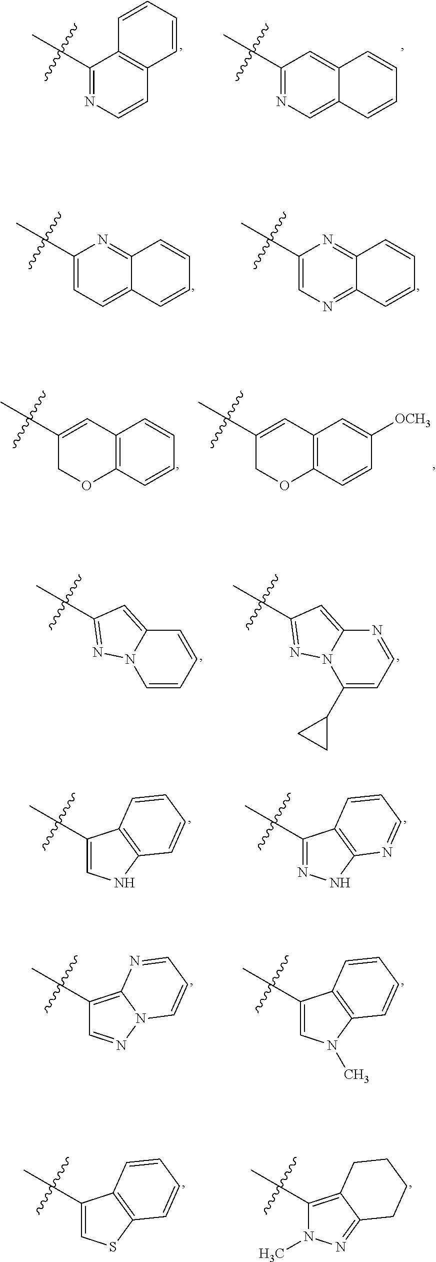

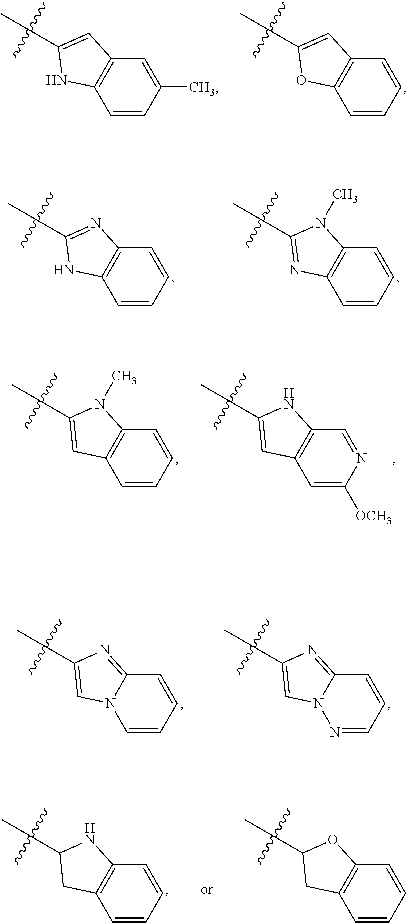

the symbol [symbol] , when drawn across a bond, indicates the point of attachment to the rest of the molecule.

190. The compound of any one of embodiments 113-180 or the pharmaceutically-acceptable salt thereof, the tautomer thereof, the pharmaceutically-acceptable salt of the tautomer, the stereoisomer thereof, or the mixture thereof, wherein R¹ is a group of formula

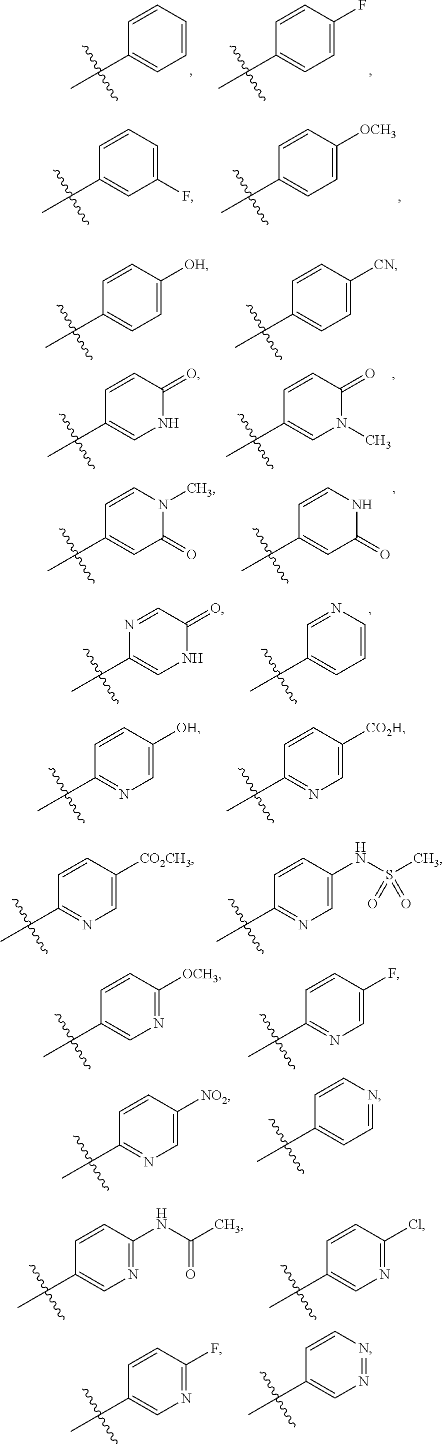

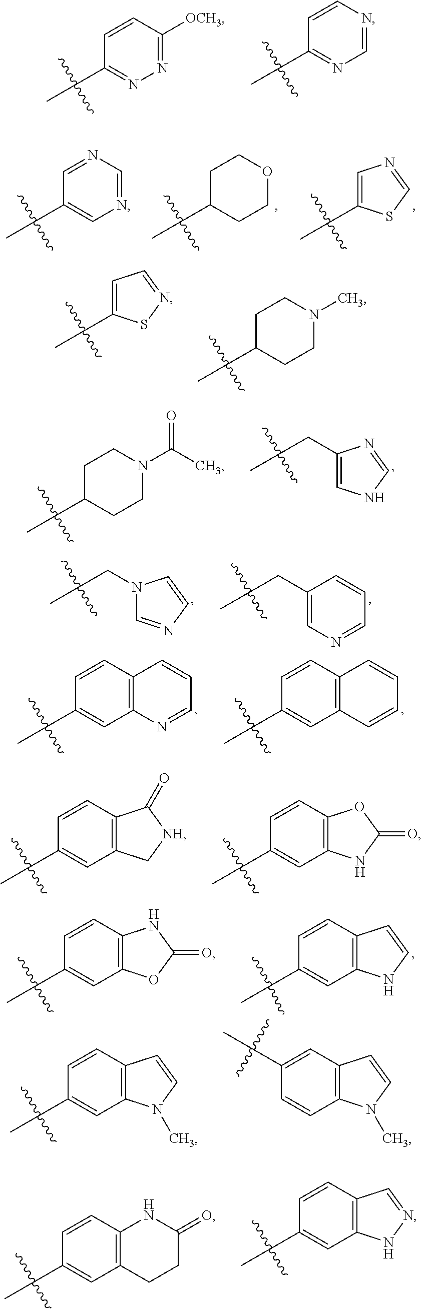

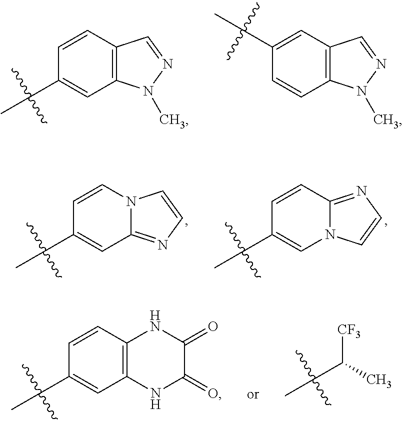

and the symbol [symbol] , when drawn across a bond, indicates the point of attachment to the rest of the molecule.

191. The compound of any one of embodiments 113-180 or the pharmaceutically-acceptable salt hereof, the tautomer thereof, the pharmaceutically-acceptable salt of the tautomer, the stereoisomer thereof, or the mixture thereof, wherein R¹ is a group of formula

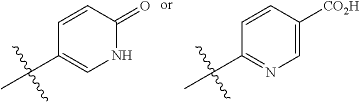 [SMILES: *c1ccc(=O)[nH]c1 ; "or" ; *c1ccc(C(=O)O)cn1]

and the symbol [symbol] , when drawn across a bond, indicates the point of attachment to the rest of the molecule.

192. The compound of any one of embodiments 113-180 or the pharmaceutically-acceptable salt thereof, the tautomer thereof, the pharmaceutically-acceptable salt of the tautomer, the stereoisomer thereof, or the mixture thereof, wherein R¹ is

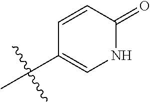 [SMILES: *c1ccc(=O)[nH]c1]

and the symbol [symbol] , when drawn across a bond, indicates the point of attachment to the rest of the molecule.

193. The compound of any one of embodiments 113-180 or the pharmaceutically-acceptable salt thereof, the tautomer thereof, the pharmaceutically-acceptable salt of the tautomer, the stereoisomer thereof, or the mixture thereof, wherein R¹ is

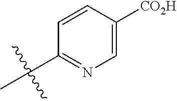 [SMILES: *c1ccc(C(=O)O)cn1]

and the symbol [symbol] , when drawn across a bond, indicates the point of attachment to the rest of the molecule.

194. The compound of any one of embodiments 113-180 or the pharmaceutically-acceptable salt thereof, the tautomer thereof, the pharmaceutically-acceptable salt of the tautomer, the stereoisomer thereof, or the mixture thereof, wherein R¹ is

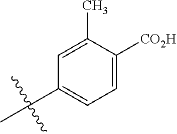 [SMILES: *c1ccc(C(=O)O)c(C)c1]

and the symbol [symbol] , when drawn across a bond, indicates the point of attachment to the rest of the molecule.

195. The compound of any one of embodiments 113-180 or the pharmaceutically-acceptable salt thereof, the tautomer thereof, the pharmaceutically-acceptable salt of the tautomer, the stereoisomer thereof, or the mixture thereof, wherein R¹ is

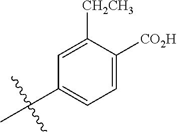 [SMILES: *c1ccc(C(=O)O)c(CC)c1]

and the symbol [symbol] , when drawn across a bond, indicates the point of attachment to the rest of the molecule.

196. The compound of any one of embodiments 113-180 or the pharmaceutically-acceptable salt thereof, the tautomer thereof, the pharmaceutically-acceptable salt of the tautomer, the stereoisomer thereof, or the mixture thereof, wherein R¹ is

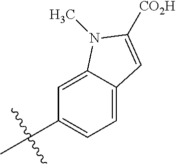 [SMILES: *c1ccc2cc(C(=O)O)n(C)c2c1]

and the symbol [symbol] , when drawn across a bond, indicates the point of attachment to the rest of the molecule.

197. The compound of embodiment 113, wherein the compound is
(S)-1-(4-(3-fluoro-4-(trifluoromethoxy)phenyl)-3,4-dihydro-2H-pyrano[3,2-b]pyridin-4-yl)-3-(pyrazin-2-yl)urea;
(S)-1-(4-(3-fluoro-4-(trifluoromethoxy)phenyl)-3,4-dihydro-2H-pyrano[3,2-b]pyridin-4-yl)-3-(pyrimidin-2-yl)urea;
(S)-methyl 6-(3-(4-(3-fluoro-4-(trifluoromethoxy)phenyl)-3,4-dihydro-2H-pyrano[3,2-b]pyridin-4-yl)ureido)nicotinate;
(S)-1-(4-(3-fluoro-4-(trifluoromethoxy)phenyl)-3,4-dihydro-2H-pyrano[3,2-b]pyridin-4-yl)-3-(pyrimidin-5-yl)urea;
(S)-methyl 2-(3-(4-(3-fluoro-4-(trifluoromethoxy)phenyl)-3,4-dihydro-2H-pyrano[3,2-b]pyridin-4-yl)ureido)benzoate;
(S)-methyl 5-(3-(4-(3-fluoro-4-(trifluoromethoxy)phenyl)-3,4-dihydro-2H-pyrano[3,2-b]pyridin-4-yl)ureido)picolinate;
(S)-1-(6-bromopyridin-3-yl)-3-(4-(3-fluoro-4-(trifluoromethoxy)phenyl)-3,4-dihydro-2H-pyrano[3,2-b]pyridin-4-yl)urea;
(S)-1-(4-(3-fluoro-4-(trifluoromethoxy)phenyl)-3,4-dihydro-2H-pyrano[3,2-b]pyridin-4-yl)-3-(pyrimidin-4-yl)urea;
(S)-methyl 4-(3-(4-(3-fluoro-4-(trifluoromethoxy)phenyl)-3,4-dihydro-2H-pyrano[3,2-b]pyridin-4-yl)ureido)benzoate;
(S)-methyl 3-(3-(4-(3-fluoro-4-(trifluoromethoxy)phenyl)-3,4-dihydro-2H-pyrano[3,2-b]pyridin-4-yl)ureido)benzoate;
(S)-1-(4-(3-fluoro-4-(trifluoromethoxy)phenyl)-3,4-dihydro-2H-pyrano[3,2-b]pyridin-4-yl)-3-(pyridin-3-yl)urea;
(S)-1-(2-bromopyridin-3-yl)-3-(4-(3-fluoro-4-(trifluoromethoxy)phenyl)-3,4-dihydro-2H-pyrano[3,2-b]pyridin-4-yl)urea;
(S)-1-(4-bromopyridin-3-yl)-3-(4-(3-fluoro-4-(trifluoromethoxy)phenyl)-3,4-dihydro-2H-pyrano[3,2-b]pyridin-4-yl)urea;
(S)-1-(4-(3-fluoro-4-(trifluoromethoxy)phenyl)-3,4-dihydro-2H-pyrano[3,2-b]pyridin-4-yl)-3-(pyridin-2-yl)urea;
(S)-1-(4-(3-fluoro-4-(trifluoromethoxy)phenyl)-3,4-dihydro-2H-pyrano[3,2-b]pyridin-4-yl)-3-(pyridin-4-yl)urea;
(S)-1-(3,5-difluoro-4-hydroxyphenyl)-3-(4-(3-fluoro-4-(trifluoromethoxy)phenyl)-3,4-dihydro-2H-pyrano[3,2-b]pyridin-4-yl)urea;
(S)—N-(4-(3-fluoro-4-(trifluoromethoxy)phenyl)-3,4-dihydro-2H-pyrano[3,2-b]pyridin-4-yl)piperidine-1-carboxamide;
(S)-methyl 1-((4-(3-fluoro-4-(trifluoromethoxy)phenyl)-3,4-dihydro-2H-pyrano[3,2-b]pyridin-4-yl)carbamoyl)piperidine-4-carboxylate;
(S)-4,4-difluoro-N-(4-(3-fluoro-4-(trifluoromethoxy)phenyl)-3,4-dihydro-2H-pyrano[3,2-b]pyridin-4-yl)piperidine-1-carboxamide;
(S)—N-(4-(3-fluoro-4-(trifluoromethoxy)phenyl)-3,4-dihydro-2H-pyrano[3,2-b]pyridin-4-yl)morpholine-4-carboxamide;
(S)—N-(4-(3-fluoro-4-(trifluoromethoxy)phenyl)-3,4-dihydro-2H-pyrano[3,2-b]pyridin-4-yl)-4-hydroxypiperidine-1-carboxamide;
(S)—N-(4-(3-fluoro-4-(trifluoromethoxy)phenyl)-3,4-dihydro-2H-pyrano[3,2-b]pyridin-4-yl)-4-oxopiperidine-1-carboxamide;
(S)—N-(4-(3-fluoro-4-(trifluoromethoxy)phenyl)-3,4-dihydro-2H-pyrano[3,2-b]pyridin-4-yl)pyrrolidine-1-carboxamide;
(S)—N-(4-(3-fluoro-4-(trifluoromethoxy)phenyl)-3,4-dihydro-2H-pyrano[3,2-b]pyridin-4-yl)azetidine-1-carboxamide;
(S)-2-(3-(4-(3-fluoro-4-(trifluoromethoxy)phenyl)-3,4-dihydro-2H-pyrano[3,2-b]pyridin-4-yl)ureido)benzoic acid;
(S)-3-(3-(4-(3-fluoro-4-(trifluoromethoxy)phenyl)-3,4-dihydro-2H-pyrano[3,2-b]pyridin-4-yl)ureido)benzoic acid;
(S)-4-(3-(4-(3-fluoro-4-(trifluoromethoxy)phenyl)-3,4-dihydro-2H-pyrano[3,2-b]pyridin-4-yl)ureido)benzoic acid;
(S)-1-(4-(3-fluoro-4-(trifluoromethoxy)phenyl)-3,4-dihydro-2H-pyrano[3,2-b]pyridin-4-yl)-3-(6-(5-oxo-4,5-dihydro-1,2,4-oxadiazol-3-yl)pyridin-3-yl)urea;
(S)-1-(4-(3-fluoro-4-(trifluoromethoxy)phenyl)-3,4-dihydro-2H-pyrano[3,2-b]pyridin-4-yl)carbamoyl)piperidine-4-carboxylic acid;
(S)—N-(4-(4-(cyanomethyl)phenyl)-3,4-dihydro-2H-pyrano[3,2-b]pyridin-4-yl)-4-fluorobenzamide;
(S)-4-fluoro-N-(4-(4-(pyridin-2-yl)phenyl)-3,4-dihydro-2H-pyrano[3,2-b]pyridin-4-yl)benzamide;
(S)—N-(4-([1,1′-biphenyl]-4-yl)-3,4-dihydro-2H-pyrano[3,2-b]pyridin-4-yl)-4-fluorobenzamide;
(S)-4-fluoro-N-(4-(4′-fluoro-[1,1′-biphenyl]-4-yl)-3,4-dihydro-2H-pyrano[3,2-b]pyridin-4-yl)benzamide;
(S)-4-fluoro-N-(4-(4-(pyridin-3-yl)phenyl)-3,4-dihydro-2H-pyrano[3,2-b]pyridin-4-yl)benzamide;
(S)-4-fluoro-N-(4-(4-(pyridin-4-yl)phenyl)-3,4-dihydro-2H-pyrano[3,2-b]pyridin-4-yl)benzamide;
(S)—N-(4-(4-(5-ethoxypyrazin-2-yl)phenyl)-3,4-dihydro-2H-pyrano[3,2-b]pyridin-4-yl)-4-fluorobenzamide;
(S)-4-fluoro-N-(4-(3′-(hydroxymethyl)-[1,1′-biphenyl]-4-yl)-3,4-dihydro-2H-pyrano[3,2-b]pyridin-4-yl)benzamide;
(S)-4-fluoro-N-(4-(4′-(methylsulfonyl)-[1,1′-biphenyl]-4-yl)-3,4-dihydro-2H-pyrano[3,2-b]pyridin-4-yl)benzamide;
(S)-4-fluoro-N-(4-(2′-hydroxy-[1,1′-biphenyl]-4-yl)-3,4-dihydro-2H-pyrano[3,2-b]pyridin-4-yl)benzamide;
(S)—N-(4-(4′-acetamido-[1,1′-biphenyl]-4-yl)-3,4-dihydro-2H-pyrano[3,2-b]pyridin-4-yl)-4-fluorobenzamide;
(S)-4-fluoro-N-(4-(4′-fluoro-3′-(hydroxymethyl)-[1,1′-biphenyl]-4-yl)-3,4-dihydro-2H-pyrano[3,2-b]pyridin-4-yl)benzamide;
(S)-4-chloro-4′-(4-(4-fluorobenzamido)-3,4-dihydro-2H-pyrano[3,2-b]pyridin-4-yl)-[1,1′-biphenyl]-3-carboxamide;
(S)-4-fluoro-N-(4-(4-(pyrimidin-5-yl)phenyl)-3,4-dihydro-2H-pyrano[3,2-b]pyridin-4-yl)benzamide;
(S)—N-(4-(4-(3,5-dimethylisoxazol-4-yl)phenyl)-3,4-dihydro-2H-pyrano[3,2-b]pyridin-4-yl)-4-fluorobenzamide;
(S)—N-(4-(3′-amino-[1,1′-biphenyl]-4-yl)-3,4-dihydro-2H-pyrano[3,2-b]pyridin-4-yl)-4-fluorobenzamide;
(S)-4-fluoro-N-(4-(4-(2-methoxypyrimidin-5-yl)phenyl)-3,4-dihydro-2H-pyrano[3,2-b]pyridin-4-yl)benzamide;
(S)-4-fluoro-N-(4-(2′-(hydroxymethyl)-[1,1′-biphenyl]-4-yl)-3,4-dihydro-2H-pyrano[3,2-b]pyridin-4-yl)benzamide;
(S)-4′-(4-(4-fluorobenzamido)-3,4-dihydro-2H-pyrano[3,2-b]pyridin-4-yl)-N,N-dimethyl-[1,1′-biphenyl]-3-carboxamide;
(S)-4-fluoro-N-(4-(4-(4-methoxypyridin-3-yl)phenyl)-3,4-dihydro-2H-pyrano[3,2-b]pyridin-4-yl)benzamide;
(S)-4-fluoro-N-(4-(4-(2-methoxypyridin-3-yl)phenyl)-3,4-dihydro-2H-pyrano[3,2-b]pyridin-4-yl)benzamide;
(S)-4-fluoro-N-(4-(4-(4-methylpyridin-3-yl)phenyl)-3,4-dihydro-2H-pyrano[3,2-b]pyridin-4-yl)benzamide;
(S)-4-fluoro-N-(4-(4′-morpholino-[1,1′-biphenyl]-4-yl)-3,4-dihydro-2H-pyrano[3,2-b]pyridin-4-yl)benzamide;
(S)-4-fluoro-N-(4-(3′-fluoro-2′-hydroxy-[1,1′-biphenyl]-4-yl)-3,4-dihydro-2H-pyrano[3,2-b]pyridin-4-yl)benzamide;
(S)-4-fluoro-N-(4-(2′-hydroxy-3′-methoxy-[1,1′-biphenyl]-4-yl)-3,4-dihydro-2H-pyrano[3,2-b]pyridin-4-yl)benzamide;
(S)-4-fluoro-N-(4-(2′-(hydroxymethyl)-4′-methoxy-[1,1′-biphenyl]-4-yl)-3,4-dihydro-2H-pyrano[3,2-b]pyridin-4-yl)benzamide;
(S)-4′-(4-(4-fluorobenzamido)-3,4-dihydro-2H-pyrano[3,2-b]pyridin-4-yl)-N,N-dimethyl-[1,1′-biphenyl]-4-carboxamide;
(S)-4′-(4-(4-fluorobenzamido)-3,4-dihydro-2H-pyrano[3,2-b]pyridin-4-yl)-N-methyl-[1,1′-biphenyl]-4-carboxamide;
(S)—N-(4-(4-(2-chloropyrimidin-5-yl)phenyl)-3,4-dihydro-2H-pyrano[3,2-b]pyridin-4-yl)-4-fluorobenzamide;
(S)-4-fluoro-N-(4-(5′-fluoro-2′-(hydroxymethyl)-[1,1′-biphenyl]-4-yl)-3,4-dihydro-2H-pyrano[3,2-b]pyridin-4-yl)benzamide;
(S)—N-(4-(4-(5-cyanopyridin-3-yl)phenyl)-3,4-dihydro-2H-pyrano[3,2-b]pyridin-4-yl)-4-fluorobenzamide;
(S)-4-fluoro-N-(4-(3′-morpholino-[1,1′-biphenyl]-4-yl)-3,4-dihydro-2H-pyrano[3,2-b]pyridin-4-yl)benzamide;
(S)—N-(4-(2′-amino-[1,1′-biphenyl]-4-yl)-3,4-dihydro-2H-pyrano[3,2-b]pyridin-4-yl)-4-fluorobenzamide;
(S)-4-fluoro-N-(4-(2′-(methylsulfonyl)-1,1′-biphenyl-4-yl)-3,4-dihydro-2H-pyrano[3,2-b]pyridin-4-yl)benzamide;
(S)-4-fluoro-N-(4-(4-(furan-3-yl)phenyl)-3,4-dihydro-2H-pyrano[3,2-b]pyridin-4-yl)benzamide;
(S)-4-fluoro-N-(4-(4-(2-methoxypyridin-4-yl)phenyl)-3,4-dihydro-2H-pyrano[3,2-b]pyridin-4-yl)benzamide;
(S)—N-(4-(4′-amino-[1,1′-biphenyl]-4-yl)-3,4-dihydro-2H-pyrano[3,2-b]pyridin-4-yl)-4-fluorobenzamide;
(S)—N-(4-(3′-amino-4′-methyl-[1,1′-biphenyl]-4-yl)-3,4-dihydro-2H-pyrano[3,2-b]pyridin-4-yl)-4-fluorobenzamide;
(S)—N-(4-(4-(2,4-dimethoxypyrimidin-5-yl)phenyl)-3,4-dihydro-2H-pyrano[3,2-b]pyridin-4-yl)-4-fluorobenzamide;
(S)-4-fluoro-N-(4-(3′-(5-methyl-1,3,4-oxadiazol-2-yl)-[1,1′-biphenyl]-4-yl)-3,4-dihydro-2H-pyrano[3,2-b]pyridin-4-yl)benzamide;
(S)-4-fluoro-N-(4-(4′-(hydroxymethyl)-[1,1′-biphenyl]-4-yl)-3,4-dihydro-2H-pyrano[3,2-b]pyridin-4-yl)benzamide;

(R)—N—((S)-4-(3-fluoro-4-(trifluoromethoxy)phenyl)-3,4-dihydro-2H-pyrano[3,2-b]pyridin-4-yl)-2-methylpropane-2-sulfinamide;
(S)—N-(4-(4′-cyano-[1,1′-biphenyl]-4-yl)-3,4-dihydro-2H-pyrano[3,2-b]pyridin-4-yl)-4-fluorobenzamide;
(S)—N-(4-(4′-cyano-[1,1′-biphenyl]-4-yl)-3,4-dihydro-2H-pyrano[3,2-b]pyridin-4-yl)benzamide;
(S)—N-(4-(4′-cyano-[1,1′-biphenyl]-4-yl)-3,4-dihydro-2H-pyrano[3,2-b]pyridin-4-yl)-2-oxo-2,3-dihydrobenzo[d]oxazole-5-carboxamide 2,2,2-trifluoroacetate;
(S)-4-acetyl-N-(4-(3-fluoro-4-(trifluoromethoxy)phenyl)-3,4-dihydro-2H-pyrano[3,2-b]pyridin-4-yl)benzamide;

N—((S)-4-(3-fluoro-4-(trifluoromethoxy)phenyl)-3,4-dihydro-2H-pyrano[3,2-b]pyridin-4-yl)-4-((R)-1-hydroxyethyl)benzamide and N—((S)-4-(3-fluoro-4-(trifluoromethoxy)phenyl)-3,4-dihydro-2H-pyrano[3,2-b]pyridin-4-yl)-4-((S)-1-hydroxyethyl)benzamide;
(S)—N-(4-(3-fluoro-4-(trifluoromethoxy)phenyl)-3,4-dihydro-2H-pyrano[3,2-b]pyridin-4-yl)-4-(hydroxymethyl)benzamide;
N—((S)-4-(3-fluoro-4-(trifluoromethoxy)phenyl)-3,4-dihydro-2H-pyrano[3,2-b]pyridin-4-yl)-4-((R)-2,2,2-trifluoro-1-hydroxyethyl)benzamide and N—((S)-4-(3-fluoro-4-(trifluoromethoxy)phenyl)-3,4-dihydro-2H-pyrano[3,2-b]pyridin-4-yl)-4-((S)-2,2,2-trifluoro-1-hydroxyethyl)benzamide;
(S)—N-(4-(3-fluoro-4-(trifluoromethoxy)phenyl)-3,4-dihydro-2H-pyrano[3,2-b]pyridin-4-yl)-4-(2,2,2-trifluoroacetyl)benzamide;
(R)-6-((3-(3-Fluoro-4-(trifluoromethoxy)phenyl)-2,3-dihydrofuro[3,2-b]pyridin-3-yl)carbamoyl)nicotinic acid;
(S)-ethyl 5-((4-(3-fluoro-4-(trifluoromethoxy)phenyl)-3,4-dihydro-2H-pyrano[3,2-b]pyridin-4-yl)carbamoyl)-1H-imidazole-2-carboxylate;
(S)—N-(4-(3-fluoro-4-(trifluoromethoxy)phenyl)-3,4-dihydro-2H-pyrano[3,2-b]pyridin-4-yl)-1-methyl-1H-imidazole-5-carboxamide;
(S)—N-(8-(3-fluoro-4-(trifluoromethoxy)phenyl)-7,8-dihydro-6H-pyrano[2,3-b]pyrazin-8-yl)-6-oxo-1,6-dihydropyridine-3-carboxamide;
(S)—N-(7-(3-fluoro-4-(trifluoromethoxy)phenyl)-5-oxo-6,7-dihydro-5H-cyclopenta[b]pyridin-7-yl)-6-oxo-1,6-dihydropyridine-3-carboxamide;
(S)-5-bromo-N-(4-(3-fluoro-4-(trifluoromethoxy)phenyl)-3,4-dihydro-2H-pyrano[3,2-b]pyridin-4-yl)-4-methylpicolinamide;
(S)-4-bromo-N-(4-(3-fluoro-4-(trifluoromethoxy)phenyl)-3,4-dihydro-2H-pyrano[3,2-b]pyridin-4-yl)-3-methylbenzamide;
(S)-4-(4,4-dimethyl-4,5-dihydrooxazol-2-yl)-N-(4-(3-fluoro-4-(trifluoromethoxy)phenyl)-3,4-dihydro-2H-pyrano[3,2-b]pyridin-4-yl)benzamide;
(S)-ethyl 6-((4-(3-fluoro-4-(trifluoromethoxy)phenyl)-3,4-dihydro-2H-pyrano[3,2-b]pyridin-4-yl)carbamoyl)-2-methylnicotinate;
(S)-5-cyano-N-(4-(3-fluoro-4-(trifluoromethoxy)phenyl)-3,4-dihydro-2H-pyrano[3,2-b]pyridin-4-yl)picolinamide;
(S)-methyl 6-((4-(3-fluoro-4-(trifluoromethyl)phenyl)-3,4-dihydro-2H-pyrano[3,2-b]pyridin-4-yl)carbamoyl)nicotinate;
(S)-methyl 6-((4-(2-fluoro-4-(trifluoromethoxy)phenyl)-3,4-dihydro-2H-pyrano[3,2-b]pyridin-4-yl)carbamoyl)nicotinate;
(S)-methyl 6-((4-(4-(trifluoromethyl)phenyl)-3,4-dihydro-2H-pyrano[3,2-b]pyridin-4-yl)carbamoyl)nicotinate;
(S)-methyl 6-((4-(4-(trifluoromethoxy)phenyl)-3,4-dihydro-2H-pyrano[3,2-b]pyridin-4-yl)carbamoyl)nicotinate;
(S)-5-((4-(3-fluoro-4-(trifluoromethoxy)phenyl)-3,4-dihydro-2H-pyrano[3,2-b]pyridin-4-yl)carbamoyl)pyrazine-2-carboxylic acid;
(S)-3-((4-(3-fluoro-4-(trifluoromethoxy)phenyl)-3,4-dihydro-2H-pyrano[3,2-b]pyridin-4-yl)carbamoyl)benzoic acid;
(S)—N-(4-(3-fluoro-4-(trifluoromethoxy)phenyl)-3,4-dihydro-2H-pyrano[3,2-b]pyridin-4-yl)-1-methyl-1H-imidazole-4-carboxamide;
(S)-2-bromo-N-(4-(3-fluoro-4-(trifluoromethoxy)phenyl)-3,4-dihydro-2H-pyrano[3,2-b]pyridin-4-yl)-1-methyl-1H-imidazole-5-carboxamide;
(S)-methyl 4-((4-(3-fluoro-4-(trifluoromethoxy)phenyl)-3,4-dihydro-2H-pyrano[3,2-b]pyridin-4-yl)carbamoyl)-2,6-dimethylbenzoate;
(S)-methyl 5-((4-(3-fluoro-4-(trifluoromethoxy)phenyl)-3,4-dihydro-2H-pyrano[3,2-b]pyridin-4-yl)carbamoyl)-2-methoxybenzoate;
(S)-methyl 6-((4-(3,4-dichlorophenyl)-3,4-dihydro-2H-pyrano[3,2-b]pyridin-4-yl)carbamoyl)nicotinate;
(S)—N-(4-(3-fluoro-4-(trifluoromethoxy)phenyl)-3,4-dihydro-2H-pyrano[3,2-b]pyridin-4-yl)-4-sulfamoylbenzamide;
(S)—N-(4-(4-fluoro-3-(trifluoromethyl)phenyl)-3,4-dihydro-2H-pyrano[3,2-b]pyridin-4-yl)-6-oxo-1,6-dihydropyridine-3-carboxamide;
(S)-3-((4-(3-fluoro-4-(trifluoromethoxy)phenyl)-3,4-dihydro-2H-pyrano[3,2-b]pyridin-4-yl)carbamoyl)-2-methoxybenzoic acid;
(S)-ethyl 4-((4-(3-fluoro-4-(trifluoromethoxy)phenyl)-3,4-dihydro-2H-pyrano[3,2-b]pyridin-4-yl)carbamoyl)-1H-pyrrole-2-carboxylate;
(S)-methyl 2-amino-4-((4-(3-fluoro-4-(trifluoromethoxy)phenyl)-3,4-dihydro-2H-pyrano[3,2-b]pyridin-4-yl)carbamoyl)benzoate;
(S)—N-(4-(3-fluoro-4-(trifluoromethoxy)phenyl)-3,4-dihydro-2H-pyrano[3,2-b]pyridin-4-yl)oxazole-2-carboxamide;
(S)—N-(4-(3-fluoro-4-(trifluoromethoxy)phenyl)-3,4-dihydro-2H-pyrano[3,2-b]pyridin-4-yl)isothiazole-4-carboxamide;
(S)-methyl 2-ethyl-4-((4-(3-fluoro-4-(trifluoromethoxy)phenyl)-3,4-dihydro-2H-pyrano[3,2-b]pyridin-4-yl)carbamoyl)benzoate;
(S)-diethyl 5-((4-(3-fluoro-4-(trifluoromethoxy)phenyl)-3,4-dihydro-2H-pyrano[3,2-b]pyridin-4-yl)carbamoyl)isophthalate;
(S)-methyl 1-(2-((4-(3-fluoro-4-(trifluoromethoxy)phenyl)-3,4-dihydro-2H-pyrano[3,2-b]pyridin-4-yl)amino)-2-oxoethyl)-1H-pyrrole-3-carboxylate;
(S)—N-1-(4-(3-fluoro-4-(trifluoromethoxy)phenyl)-3,4-dihydro-2H-pyrano[3,2-b]pyridin-4-yl)terephthalamide;
(S)-methyl 6-((4-(3-fluoro-4-(trifluoromethoxy)phenyl)-3,4-dihydro-2H-pyrano[3,2-b]pyridin-4-yl)carbamoyl)-1-methyl-1H-indole-2-carboxylate;
(S)-2-chloro-4-((4-(3-fluoro-4-(trifluoromethoxy)phenyl)-3,4-dihydro-2H-pyrano[3,2-b]pyridin-4-yl)carbamoyl)benzoic acid;
(S)—N-(4-(3-fluoro-4-(trifluoromethoxy)phenyl)-3,4-dihydro-2H-pyrano[3,2-b]pyridin-4-yl)thiazole-4-carboxamide;
(S)—N-(4-(3-methyl-4-(trifluoromethoxy)phenyl)-3,4-dihydro-2H-pyrano[3,2-b]pyridin-4-yl)-6-oxo-1,6-dihydropyridine-3-carboxamide;
(S)-5-chloro-N-(4-(3-fluoro-4-(trifluoromethoxy)phenyl)-3,4-dihydro-2H-pyrano[3,2-b]pyridin-4-yl)isoxazole-3-carboxamide;
(S)-methyl 2-((4-(3-fluoro-4-(trifluoromethoxy)phenyl)-3,4-dihydro-2H-pyrano[3,2-b]pyridin-4-yl)carbamoyl)isonicotinate;
N—((S)-4-(3-fluoro-4-(trifluoromethoxy)phenyl)-3,4-dihydro-2H-pyrano[3,2-b]pyridin-4-yl)-1-((R)-2-hydroxypropyl)-6-oxo-1,6-dihydropyridine-3-carboxamide;
(S)-5-bromo-N-(4-(3-fluoro-4-(trifluoromethoxy)phenyl)-3,4-dihydro-2H-pyrano[3,2-b]pyridin-4-yl)thiophene-2-carboxamide;
(S)-4-((4-(3-fluoro-4-(trifluoromethoxy)phenyl)-3,4-dihydro-2H-pyrano[3,2-b]pyridin-4-yl)carbamoyl)benzoic acid;
(S)-3,5-difluoro-4-hydroxy-N-(4-(4-(trifluoromethyl)phenyl)-3,4-dihydro-2H-pyrano[3,2-b]pyridin-4-yl)benzamide;
(S)-5-bromo-N-(4-(3-fluoro-4-(trifluoromethoxy)phenyl)-3,4-dihydro-2H-pyrano[3,2-b]pyridin-4-yl)picolinamide;
(S)-3,5-difluoro-N-(4-(3-fluoro-4-(trifluoromethoxy)phenyl)-3,4-dihydro-2H-pyrano[3,2-b]pyridin-4-yl)-4-hydroxybenzamide;
(S)-5-bromo-N-(4-(3-fluoro-4-(trifluoromethoxy)phenyl)-3,4-dihydro-2H-pyrano[3,2-b]pyridin-4-yl)furan-2-carboxamide;
(S)-methyl 6-((4-(3-fluoro-4-(trifluoromethoxy)phenyl)-3,4-dihydro-2H-pyrano[3,2-b]pyridin-4-yl)carbamoyl)-1H-indole-2-carboxylate;
(S)—N-(4-(4-(trifluoromethoxy)phenyl)-3,4-dihydro-2H-pyrano[3,2-b]pyridin-4-yl)-1H-imidazole-2-carboxamide;
(S)—N-(4-(4-(trifluoromethoxy)phenyl)-3,4-dihydro-2H-pyrano[3,2-b]pyridin-4-yl)-1H-pyrazole-3-carboxamide;
(S)—N-(4-(4-(trifluoromethoxy)phenyl)-3,4-dihydro-2H-pyrano[3,2-b]pyridin-4-yl)-4H-1,2,4-triazole-3-carboxamide;
(S)—N-(4-(4-(trifluoromethoxy)phenyl)-3,4-dihydro-2H-pyrano[3,2-b]pyridin-4-yl)isothiazole-5-carboxamide;
(S)—N-(4-(4-(trifluoromethoxy)phenyl)-3,4-dihydro-2H-pyrano[3,2-b]pyridin-4-yl)-1,2,3-thiadiazole-5-carboxamide;
(S)—N-(4-(4-(trifluoromethoxy)phenyl)-3,4-dihydro-2H-pyrano[3,2-b]pyridin-4-yl)-1H-indazole-5-carboxamide;
(S)—N-(4-(4-(trifluoromethoxy)phenyl)-3,4-dihydro-2H-pyrano[3,2-b]pyridin-4-yl)-1H-pyrrolo[2,3-b]pyridine-2-carboxamide;
(S)—N-(4-(4-(trifluoromethoxy)phenyl)-3,4-dihydro-2H-pyrano[3,2-b]pyridin-4-yl)-1H-pyrrolo[2,3-b]pyridine-3-carboxamide;
(S)—N-(4-(4-(trifluoromethoxy)phenyl)-3,4-dihydro-2H-pyrano[3,2-b]pyridin-4-yl)-1H-pyrrolo[2,3-b]pyridine-4-carboxamide;
(S)—N-(4-(4-(trifluoromethoxy)phenyl)-3,4-dihydro-2H-pyrano[3,2-b]pyridin-4-yl)pyrazolo[1,5-a]pyridine-2-carboxamide;
(S)—N-(4-(4-(trifluoromethoxy)phenyl)-3,4-dihydro-2H-pyrano[3,2-b]pyridin-4-yl)-6,7-dihydro-5H-pyrazolo[5,1-b][1,3]oxazine-2-carboxamide;
(S)—N-(4-(4-(trifluoromethoxy)phenyl)-3,4-dihydro-2H-pyrano[3,2-b]pyridin-4-yl)quinoline-8-carboxamide;
(S)-5-methyl-N-(4-(4-(trifluoromethoxy)phenyl)-3,4-dihydro-2H-pyrano[3,2-b]pyridin-4-yl)-1H-indole-2-carboxamide;
(S)-1-oxo-N-(4-(4-(trifluoromethoxy)phenyl)-3,4-dihydro-2H-pyrano[3,2-b]pyridin-4-yl)-1,2-dihydroisoquinoline-6-carboxamide;
(S)-7-cyclopropyl-N-(4-(4-(trifluoromethoxy)phenyl)-3,4-dihydro-2H-pyrano[3,2-b]pyridin-4-yl)pyrazolo[1,5-a]pyrimidine-2-carboxamide;
(S)-7-methoxy-N-(4-(4-(trifluoromethoxy)phenyl)-3,4-dihydro-2H-pyrano[3,2-b]pyridin-4-yl)quinoline-3-carboxamide;
(S)—N-(4-(4-(trifluoromethoxy)phenyl)-3,4-dihydro-2H-pyrano[3,2-b]pyridin-4-yl)oxazole-4-carboxamide;
(S)-1-methyl-N-(4-(4-(trifluoromethoxy)phenyl)-3,4-dihydro-2H-pyrano[3,2-b]pyridin-4-yl)-1H-pyrazole-3-carboxamide;
(S)-1-methyl-N-(4-(4-(trifluoromethoxy)phenyl)-3,4-dihydro-2H-pyrano[3,2-b]pyridin-4-yl)-1H-pyrazole-5-carboxamide;
(S)—N-(4-(4-(trifluoromethoxy)phenyl)-3,4-dihydro-2H-pyrano[3,2-b]pyridin-4-yl)thiophene-2-carboxamide;
(S)—N-(4-(4-(trifluoromethoxy)phenyl)-3,4-dihydro-2H-pyrano[3,2-b]pyridin-4-yl)thiophene-3-carboxamide;
(S)—N-(4-(4-(trifluoromethoxy)phenyl)-3,4-dihydro-2H-pyrano[3,2-b]pyridin-4-yl)thiazole-4-carboxamide;
(S)—N-(4-(4-(trifluoromethoxy)phenyl)-3,4-dihydro-2H-pyrano[3,2-b]pyridin-4-yl)isothiazole-3-carboxamide;
(S)—N-(4-(4-(trifluoromethoxy)phenyl)-3,4-dihydro-2H-pyrano[3,2-b]pyridin-4-yl)thiazole-5-carboxamide;
(S)—N-(4-(4-(trifluoromethoxy)phenyl)-3,4-dihydro-2H-pyrano[3,2-b]pyridin-4-yl)-1H-indole-3-carboxamide;
(S)—N-(4-(4-(trifluoromethoxy)phenyl)-3,4-dihydro-2H-pyrano[3,2-b]pyridin-4-yl)-1H-indole-6-carboxamide;
(S)—N-(4-(4-(trifluoromethoxy)phenyl)-3,4-dihydro-2H-pyrano[3,2-b]pyridin-4-yl)benzofuran-2-carboxamide;
(S)—N-(4-(4-(trifluoromethoxy)phenyl)-3,4-dihydro-2H-pyrano[3,2-b]pyridin-4-yl)-1H-benzo[d]imidazole-5-carboxamide;
(S)—N-(4-(4-(trifluoromethoxy)phenyl)-3,4-dihydro-2H-pyrano[3,2-b]pyridin-4-yl)-1H-benzo[d]imidazole-4-carboxamide;
(S)—N-(4-(4-(trifluoromethoxy)phenyl)-3,4-dihydro-2H-pyrano[3,2-b]pyridin-4-yl)imidazo[1,2-a]pyridine-2-carboxamide;
(S)—N-(4-(4-(trifluoromethoxy)phenyl)-3,4-dihydro-2H-pyrano[3,2-b]pyridin-4-yl)imidazo[1,2-a]pyridine-6-carboxamide;
(S)—N-(4-(4-(trifluoromethoxy)phenyl)-3,4-dihydro-2H-pyrano[3,2-b]pyridin-4-yl)-1H-benzo[d]imidazole-2-carboxamide;
(S)—N-(4-(4-(trifluoromethoxy)phenyl)-3,4-dihydro-2H-pyrano[3,2-b]pyridin-4-yl)-1H-pyrazolo[3,4-b]pyridine-3-carboxamide;
(S)—N-(4-(4-(trifluoromethoxy)phenyl)-3,4-dihydro-2H-pyrano[3,2-b]pyridin-4-yl)imidazo[1,2-b]pyridazine-2-carboxamide;
(S)—N-(4-(4-(trifluoromethoxy)phenyl)-3,4-dihydro-2H-pyrano[3,2-b]pyridin-4-yl)pyrazolo[1,5-a]pyrimidine-3-carboxamide;
N—((S)-4-(4-(trifluoromethoxy)phenyl)-3,4-dihydro-2H-pyrano[3,2-b]pyridin-4-yl)indoline-2-carboxamide;
N—((S)-4-(4-(trifluoromethoxy)phenyl)-3,4-dihydro-2H-pyrano[3,2-b]pyridin-4-yl)-2,3-dihydrobenzofuran-2-carboxamide;
(S)—N-(4-(4-(trifluoromethoxy)phenyl)-3,4-dihydro-2H-pyrano[3,2-b]pyridin-4-yl)isoquinoline-1-carboxamide;
(S)—N-(4-(4-(trifluoromethoxy)phenyl)-3,4-dihydro-2H-pyrano[3,2-b]pyridin-4-yl)isoquinoline-3-carboxamide;
(S)—N-(4-(4-(trifluoromethoxy)phenyl)-3,4-dihydro-2H-pyrano[3,2-b]pyridin-4-yl)isoquinoline-5-carboxamide;
(S)—N-(4-(4-(trifluoromethoxy)phenyl)-3,4-dihydro-2H-pyrano[3,2-b]pyridin-4-yl)isoquinoline-6-carboxamide;
(S)—N-(4-(4-(trifluoromethoxy)phenyl)-3,4-dihydro-2H-pyrano[3,2-b]pyridin-4-yl)quinoline-2-carboxamide;
(S)—N-(4-(4-(trifluoromethoxy)phenyl)-3,4-dihydro-2H-pyrano[3,2-b]pyridin-4-yl)quinoline-3-carboxamide;
(S)—N-(4-(4-(trifluoromethoxy)phenyl)-3,4-dihydro-2H-pyrano[3,2-b]pyridin-4-yl)quinoline-6-carboxamide;
(S)—N-(4-(4-(trifluoromethoxy)phenyl)-3,4-dihydro-2H-pyrano[3,2-b]pyridin-4-yl)quinoline-7-carboxamide;
(S)—N-(4-(4-(trifluoromethoxy)phenyl)-3,4-dihydro-2H-pyrano[3,2-b]pyridin-4-yl)quinoxaline-2-carboxamide;
(S)-1-methyl-N-(4-(4-(trifluoromethoxy)phenyl)-3,4-dihydro-2H-pyrano[3,2-b]pyridin-4-yl)-1H-indole-2-carboxamide;
(S)-1-methyl-N-(4-(4-(trifluoromethoxy)phenyl)-3,4-dihydro-2H-pyrano[3,2-b]pyridin-4-yl)-1H-indole-3-carboxamide;
(S)-1-methyl-N-(4-(4-(trifluoromethoxy)phenyl)-3,4-dihydro-2H-pyrano[3,2-b]pyridin-4-yl)-1H-indole-5-carboxamide;
(S)—N-(4-(4-(trifluoromethoxy)phenyl)-3,4-dihydro-2H-pyrano[3,2-b]pyridin-4-yl)-2H-chromene-3-carboxamide;
(S)-1-methyl-N-(4-(4-(trifluoromethoxy)phenyl)-3,4-dihydro-2H-pyrano[3,2-b]pyridin-4-yl)-1H-benzo[d]imidazole-2-carboxamide;
(S)-1-methyl-N-(4-(4-(trifluoromethoxy)phenyl)-3,4-dihydro-2H-pyrano[3,2-b]pyridin-4-yl)-1H-indazole-4-carboxamide;
(S)-1-methyl-N-(4-(4-(trifluoromethoxy)phenyl)-3,4-dihydro-2H-pyrano[3,2-b]pyridin-4-yl)-1H-indazole-5-carboxamide;
(S)-1-methyl-N-(4-(4-(trifluoromethoxy)phenyl)-3,4-dihydro-2H-pyrano[3,2-b]pyridin-4-yl)-1H-indazole-6-carboxamide;
(S)-2-methyl-N-(4-(4-(trifluoromethoxy)phenyl)-3,4-dihydro-2H-pyrano[3,2-b]pyridin-4-yl)-2H-indazole-4-carboxamide;
(S)-2-methyl-N-(4-(4-(trifluoromethoxy)phenyl)-3,4-dihydro-2H-pyrano[3,2-b]pyridin-4-yl)-2H-indazole-6-carboxamide;
(S)-2-methyl-N-(4-(4-(trifluoromethoxy)phenyl)-3,4-dihydro-2H-pyrano[3,2-b]pyridin-4-yl)-2H-indazole-7-carboxamide;
(S)—N-(4-(4-(trifluoromethoxy)phenyl)-3,4-dihydro-2H-pyrano[3,2-b]pyridin-4-yl)benzo[b]thiophene-2-carboxamide;
(S)—N-(4-(4-(trifluoromethoxy)phenyl)-3,4-dihydro-2H-pyrano[3,2-b]pyridin-4-yl)benzo[b]thiophene-3-carboxamide;
(S)—N-(4-(4-(trifluoromethoxy)phenyl)-3,4-dihydro-2H-pyrano[3,2-b]pyridin-4-yl)benzo[c][1,2,5]thiadiazole-5-carboxamide;
(S)-2-methyl-N-(4-(4-(trifluoromethoxy)phenyl)-3,4-dihydro-2H-pyrano[3,2-b]pyridin-4-yl)-4,5,6,7-tetrahydro-2H-indazole-3-carboxamide;
(S)-1-oxo-N-(4-(4-(trifluoromethoxy)phenyl)-3,4-dihydro-2H-pyrano[3,2-b]pyridin-4-yl)-1,2-dihydroisoquinoline-7-carboxamide;
(S)-1-oxo-N-(4-(4-(trifluoromethoxy)phenyl)-3,4-dihydro-2H-pyrano[3,2-b]pyridin-4-yl)-1,2-dihydroisoquinoline-4-carboxamide;
(S)-4-(oxazol-5-yl)-N-(4-(4-(trifluoromethoxy)phenyl)-3,4-dihydro-2H-pyrano[3,2-b]pyridin-4-yl)benzamide;
(S)-5-methoxy-N-(4-(4-(trifluoromethoxy)phenyl)-3,4-dihydro-2H-pyrano[3,2-b]pyridin-4-yl)-1H-pyrrolo[2,3-c]pyridine-2-carboxamide;
(S)-3-(5-methyl-1,2,4-oxadiazol-3-yl)-N-(4-(4-(trifluoromethoxy)phenyl)-3,4-dihydro-2H-pyrano[3,2-b]pyridin-4-yl)benzamide;
(S)-4-(5-methyl-1,2,4-oxadiazol-3-yl)-N-(4-(4-(trifluoromethoxy)phenyl)-3,4-dihydro-2H-pyrano[3,2-b]pyridin-4-yl)benzamide;
(S)-3-(2-oxopyrrolidin-1-yl)-N-(4-(4-(trifluoromethoxy)phenyl)-3,4-dihydro-2H-pyrano[3,2-b]pyridin-4-yl)benzamide;
(S)-6-methoxy-N-(4-(4-(trifluoromethoxy)phenyl)-3,4-dihydro-2H-pyrano[3,2-b]pyridin-4-yl)-2H-chromene-3-carboxamide;
(S)-ethyl 4-((4-(3-fluoro-4-(trifluoromethoxy)phenyl)-3,4-dihydro-2H-pyrano[3,2-b]pyridin-4-yl)carbamoyl)thiazole-2-carboxylate;
(S)-methyl 3-((4-(3-fluoro-4-(trifluoromethoxy)phenyl)-3,4-dihydro-2H-pyrano[3,2-b]pyridin-4-yl)carbamoyl)benzoate;
(S)—N-(4-(3-fluoro-4-(trifluoromethoxy)phenyl)-3,4-dihydro-2H-pyrano[3,2-b]pyridin-4-yl)-5-(2H-tetrazol-5-yl)picolinamide;
(S)—N-(4-(3-fluoro-4-(trifluoromethoxy)phenyl)-3,4-dihydro-2H-pyrano[3,2-b]pyridin-4-yl)-5-(1,2,4-oxadiazol-3-yl)picolinamide;
(S)—N-(4-(3-fluoro-4-(trifluoromethoxy)phenyl)-3,4-dihydro-2H-pyrano[3,2-b]pyridin-4-yl)-5-(5-oxo-4,5-dihydro-1,2,4-oxadiazol-3-yl)picolinamide;
(S)-5-amino-N-(4-(3-fluoro-4-(trifluoromethoxy)phenyl)-3,4-dihydro-2H-pyrano[3,2-b]pyridin-4-yl)picolinamide;
(S)-methyl 3-(N-(4-(3-fluoro-4-(trifluoromethoxy)phenyl)-3,4-dihydro-2H-pyrano[3,2-b]pyridin-4-yl)sulfamoyl)benzoate;
(S)-methyl 4-(N-(4-(3-fluoro-4-(trifluoromethoxy)phenyl)-3,4-dihydro-2H-pyrano[3,2-b]pyridin-4-yl)sulfamoyl)benzoate;
(S)-4-((4-(3-fluoro-4-(trifluoromethyl)phenyl)-3,4-dihydro-2H-pyrano[3,2-b]pyridin-4-yl)carbamoyl)-2-methylbenzoic acid;
(S)-2-methyl-4-((4-(4-(trifluoromethyl)phenyl)-3,4-dihydro-2H-pyrano[3,2-b]pyridin-4-yl)carbamoyl)benzoic acid;
(S)-4-((4-(3-fluoro-4-(trifluoromethoxy)phenyl)-3,4-dihydro-2H-pyrano[3,2-b]pyridin-4-yl)carbamoyl)-2-vinylbenzoic acid;
(S)-5-((4-(3-fluoro-4-(trifluoromethoxy)phenyl)-3,4-dihydro-2H-pyrano[3,2-b]pyridin-4-yl)carbamoyl)-[1,1′-biphenyl]-2-carboxylic acid;
(S)-2-ethyl-4-((4-(3-fluoro-4-(trifluoromethoxy)phenyl)-3,4-dihydro-2H-pyrano[3,2-b]pyridin-4-yl)carbamoyl)benzoic acid;
(S)-4-((4-(3-fluoro-4-(trifluoromethoxy)phenyl)-3,4-dihydro-2H-pyrano[3,2-b]pyridin-4-yl)carbamoyl)-2-isopropylbenzoic acid;
(S)-4-((4-(3-fluoro-4-(trifluoromethoxy)phenyl)-3,4-dihydro-2H-pyrano[3,2-b]pyridin-4-yl)carbamoyl)-2-isobutylbenzoic acid;
(S)-4-((4-(3-fluoro-4-(trifluoromethoxy)phenyl)-3,4-dihydro-2H-pyrano[3,2-b]pyridin-4-yl)carbamoyl)-2-(methylamino)benzoic acid;
(S)-methyl 2-acetamido-4-((4-(3-fluoro-4-(trifluoromethoxy)phenyl)-3,4-dihydro-2H-pyrano[3,2-b]pyridin-4-yl)carbamoyl)benzoate;
(S)—N4-(4-(3-fluoro-4-(trifluoromethoxy)phenyl)-3,4-dihydro-2H-pyrano[3,2-b]pyridin-4-yl)-N1,2-dimethylterephthalamide;
(S)—N1-(tert-butyl)-N4-(4-(3-fluoro-4-(trifluoromethoxy)phenyl)-3,4-dihydro-2H-pyrano[3,2-b]pyridin-4-yl)-2-methylterephthalamide;
(S)-4-((4-(3-fluoro-4-(trifluoromethoxy)phenyl)-3,4-dihydro-2H-pyrano[3,2-b]pyridin-4-yl)carbamoyl)-2-hydroxybenzoic acid;
(S)-4-((4-(3-fluoro-4-(trifluoromethoxy)phenyl)-3,4-dihydro-2H-pyrano[3,2-b]pyridin-4-yl)carbamoyl)-1-methyl-1H-pyrrole-2-carboxylic acid;
(S)-2-cyclopropyl-4-((4-(3-fluoro-4-(trifluoromethoxy)phenyl)-3,4-dihydro-2H-pyrano[3,2-b]pyridin-4-yl)carbamoyl)benzoic acid;
(S)-5-((4-(3-fluoro-4-(trifluoromethoxy)phenyl)-3,4-dihydro-2H-pyrano[3,2-b]pyridin-4-yl)carbamoyl)-2-methyl-1H-pyrrole-3-carboxylic acid;
(S)-2-(cyclopropylamino)-4-((4-(3-fluoro-4-(trifluoromethoxy)phenyl)-3,4-dihydro-2H-pyrano[3,2-b]pyridin-4-yl)carbamoyl)benzoic acid;
(S)-5-((4-(3-fluoro-4-(trifluoromethoxy)phenyl)-3,4-dihydro-2H-pyrano[3,2-b]pyridin-4-yl)carbamoyl)-1,2-dimethyl-1H-pyrrole-3-carboxylic acid;
(S)-2-fluoro-4-((4-(3-fluoro-4-(trifluoromethoxy)phenyl)-3,4-dihydro-2H-pyrano[3,2-b]pyridin-4-yl)carbamoyl)-6-(methylamino)benzoic acid;
(S)-methyl 5-((4-(3-fluoro-4-(trifluoromethoxy)phenyl)-3,4-dihydro-2H-pyrano[3,2-b]pyridin-4-yl)carbamoyl)-1-methyl-1H-pyrrole-2-carboxylate;
(S)-5-((4-(3-fluoro-4-(trifluoromethoxy)phenyl)-3,4-dihydro-2H-pyrano[3,2-b]pyridin-4-yl)carbamoyl)-1H-pyrrole-2-carboxylic acid;
(S)—N2-(4-(3-fluoro-4-(trifluoromethoxy)phenyl)-3,4-dihydro-2H-pyrano[3,2-b]pyridin-4-yl)-N5-(phenylsulfonyl)pyridine-2,5-dicarboxamide;
(S)—N-(4-(3-fluoro-4-(trifluoromethoxy)phenyl)-3,4-dihydro-2H-pyrano[3,2-b]pyridin-4-yl)furan-2-carboxamide;
(S)—N1-(4-(3-fluoro-4-(trifluoromethoxy)phenyl)-3,4-dihydro-2H-pyrano[3,2-b]pyridin-4-yl)-N3-methylisophthalamide;
(S)-6-((4-(3-fluoro-4-(trifluoromethoxy)phenyl)-3,4-dihydro-2H-pyrano[3,2-b]pyridin-4-yl)carbamoyl)-2-methylnicotinic acid;
(S)-4-((4-(3-fluoro-4-(trifluoromethoxy)phenyl)-3,4-dihydro-2H-pyrano[3,2-b]pyridin-4-yl)carbamoyl)-2,6-dimethylbenzoic acid;
(S)-4-((4-(3-fluoro-4-(trifluoromethoxy)phenyl)-3,4-dihydro-2H-pyrano[3,2-b]pyridin-4-yl)carbamoyl)-3-methoxybenzoic acid;
(S)-5-((4-(3-fluoro-4-(trifluoromethoxy)phenyl)-3,4-dihydro-2H-pyrano[3,2-b]pyridin-4-yl)carbamoyl)-2-methoxybenzoic acid;
(S)-3-((4-(3-fluoro-4-(trifluoromethoxy)phenyl)-3,4-dihydro-2H-pyrano[3,2-b]pyridin-4-yl)carbamoyl)-4-methoxybenzoic acid;
(S)-4-(N-(4-(3-fluoro-4-(trifluoromethoxy)phenyl)-3,4-dihydro-2H-pyrano[3,2-b]pyridin-4-yl)sulfamoyl)benzoic acid;
(S)-2-methyl-4-((4-(4-(trifluoromethoxy)phenyl)-3,4-dihydro-2H-pyrano[3,2-b]pyridin-4-yl)carbamoyl)benzoic acid;
(S)-4-((4-(3-fluoro-4-(trifluoromethoxy)phenyl)-3,4-dihydro-2H-pyrano[3,2-b]pyridin-4-yl)carbamoyl)-1H-pyrrole-2-carboxylic acid;
(S)-2-amino-4-((4-(3-fluoro-4-(trifluoromethoxy)phenyl)-3,4-dihydro-2H-pyrano[3,2-b]pyridin-4-yl)carbamoyl)benzoic acid;
(S)-6-((4-(3-fluoro-4-(trifluoromethoxy)phenyl)-3,4-dihydro-2H-pyrano[3,2-b]pyridin-4-yl)carbamoyl)-1-methyl-1H-indole-2-carboxylic acid;
(S)-2-acetamido-4-((4-(3-fluoro-4-(trifluoromethoxy)phenyl)-3,4-dihydro-2H-pyrano[3,2-b]pyridin-4-yl)carbamoyl)benzoic acid;
(S)-5-((4-(3-fluoro-4-(trifluoromethoxy)phenyl)-3,4-dihydro-2H-pyrano[3,2-b]pyridin-4-yl)carbamoyl)-1-methyl-1H-pyrrole-2-carboxylic acid;
(S)-6-((4-(3-fluoro-4-(trifluoromethoxy)phenyl)-3,4-dihydro-2H-pyrano[3,2-b]pyridin-4-yl)carbamoyl)-1H-indole-2-carboxylic acid;
(S)-5-((4-(3-fluoro-4-(trifluoromethoxy)phenyl)-3,4-dihydro-2H-pyrano[3,2-b]pyridin-4-yl)carbamoyl)picolinic acid;
(S)-4-((4-(3-fluoro-4-(trifluoromethoxy)phenyl)-3,4-dihydro-2H-pyrano[3,2-b]pyridin-4-yl)carbamoyl)thiazole-2-carboxylic acid;
(S)-2-((4-(3-fluoro-4-(trifluoromethoxy)phenyl)-3,4-dihydro-2H-pyrano[3,2-b]pyridin-4-yl)carbamoyl)isonicotinic acid;
(S)-2-fluoro-4-((4-(3-fluoro-4-(trifluoromethoxy)phenyl)-3,4-dihydro-2H-pyrano[3,2-b]pyridin-4-yl)carbamoyl)benzoic acid;
(S)-4-((4-(3-fluoro-4-(trifluoromethoxy)phenyl)-3,4-dihydro-2H-pyrano[3,2-b]pyridin-4-yl)carbamoyl)-2-methylbenzoic acid;
(S)-6-((4-(3-fluoro-4-(trifluoromethoxy)phenyl)-3,4-dihydro-2H-pyrano[3,2-b]pyridin-4-yl)carbamoyl)-4-methylnicotinic acid;
(S)-5-((4-(3-fluoro-4-(trifluoromethoxy)phenyl)-3,4-dihydro-2H-pyrano[3,2-b]pyridin-4-yl)carbamoyl)-1H-pyrrole-3-carboxylic acid;
(S)-5-((4-(3-fluoro-4-(trifluoromethoxy)phenyl)-3,4-dihydro-2H-pyrano[3,2-b]pyridin-4-yl)carbamoyl)-1-methyl-1H-pyrrole-3-carboxylic acid;
(S)-5-((4-(3-fluoro-4-(trifluoromethoxy)phenyl)-3,4-dihydro-2H-pyrano[3,2-b]pyridin-4-yl)carbamoyl)thiophene-2-carboxylic acid; or
(S)-4-((4-(3-fluoro-4-(trifluoromethoxy)phenyl)-3,4-dihydro-2H-pyrano[3,2-b]pyridin-4-yl)carbamoyl)-2-methoxybenzoic acid; or
   the pharmaceutically-acceptable salt thereof, the tautomer thereof, the pharmaceutically-acceptable salt of the tautomer, the stereoisomer thereof, or the mixture thereof.

198. The compound of embodiment 113, wherein the compound is
(S)-1-(4-(3-fluoro-4-(trifluoromethoxy)phenyl)-3,4-dihydro-2H-pyrano[3,2-b]pyridin-4-yl)-3-(pyrimidin-2-yl)urea;
(S)-1-(4-(3-fluoro-4-(trifluoromethoxy)phenyl)-3,4-dihydro-2H-pyrano[3,2-b]pyridin-4-yl)-3-(pyrimidin-5-yl)urea;
(S)—N-(4-(4′-cyano-[1,1′-biphenyl]-4-yl)-3,4-dihydro-2H-pyrano[3,2-b]pyridin-4-yl)-2-oxo-2,3-dihydrobenzo[d]oxazole-5-carboxamide;
(S)—N-(4-(3-fluoro-4-(trifluoromethoxy)phenyl)-3,4-dihydro-2H-pyrano[3,2-b]pyridin-4-yl)-4-(hydroxymethyl)benzamide;
(S)-4-((4-(3-fluoro-4-(trifluoromethoxy)phenyl)-3,4-dihydro-2H-pyrano[3,2-b]pyridin-4-yl)carbamoyl)benzoic acid;
(S)—N-(4-(3-fluoro-4-(trifluoromethoxy)phenyl)-3,4-dihydro-2H-pyrano[3,2-b]pyridin-4-yl)-5-(5-oxo-4,5-dihydro-1,2,4-oxadiazol-3-yl)picolinamide;
(S)-4-((4-(3-fluoro-4-(trifluoromethoxy)phenyl)-3,4-dihydro-2H-pyrano[3,2-b]pyridin-4-yl)carbamoyl)-2-vinylbenzoic acid;
(S)-2-ethyl-4-((4-(3-fluoro-4-(trifluoromethoxy)phenyl)-3,4-dihydro-2H-pyrano[3,2-b]pyridin-4-yl)carbamoyl)benzoic acid;
(S)-6-((4-(3-fluoro-4-(trifluoromethoxy)phenyl)-3,4-dihydro-2H-pyrano[3,2-b]pyridin-4-yl)carbamoyl)-2-methylnicotinic acid;
(S)-4-((4-(3-fluoro-4-(trifluoromethoxy)phenyl)-3,4-dihydro-2H-pyrano[3,2-b]pyridin-4-yl)carbamoyl)-3-methoxybenzoic acid;
(S)-2-methyl-4-((4-(4-(trifluoromethoxy)phenyl)-3,4-dihydro-2H-pyrano[3,2-b]pyridin-4-yl)carbamoyl)benzoic acid;
(S)-2-amino-4-((4-(3-fluoro-4-(trifluoromethoxy)phenyl)-3,4-dihydro-2H-pyrano[3,2-b]pyridin-4-yl)carbamoyl)benzoic acid;
(S)-6-((4-(3-fluoro-4-(trifluoromethoxy)phenyl)-3,4-dihydro-2H-pyrano[3,2-b]pyridin-4-yl)carbamoyl)-1-methyl-1H-indole-2-carboxylic acid; or
(S)-4-((4-(3-fluoro-4-(trifluoromethoxy)phenyl)-3,4-dihydro-2H-pyrano[3,2-b]pyridin-4-yl)carbamoyl)-2-methylbenzoic acid; or
   the pharmaceutically-acceptable salt thereof, the tautomer thereof, the pharmaceutically-acceptable salt of the tautomer, the stereoisomer thereof, or the mixture thereof.

199. The compound of embodiment 113, wherein the compound is (S)—N-(4-(4′-cyano-[1,1′-biphenyl]-4-yl)-3,4-dihydro-2H-pyrano[3,2-b]pyridin-4-yl)-2-oxo-2,3-dihydrobenzo[d]oxazole-5-carboxamide, or the pharmaceutically-acceptable salt thereof, the tautomer thereof, the pharmaceutically-acceptable salt of the tautomer, the stereoisomer thereof, or the mixture thereof.

200. The compound of embodiment 113, wherein the compound is (S)-1-(4-(3-fluoro-4-(trifluoromethoxy)phenyl)-3,4-dihydro-2H-pyrano[3,2-b]pyridin-4-yl)-3-(pyrimidin-2-yl)urea, or the pharmaceutically-acceptable salt thereof, the tautomer thereof, the pharmaceutically-acceptable salt of the tautomer, the stereoisomer thereof, or the mixture thereof.

201. The compound of embodiment 113, wherein the compound is (S)—N-(4-(3-fluoro-4-(trifluoromethoxy)phenyl)-3,4-dihydro-2H-pyrano[3,2-b]pyridin-4-yl)-5-(5-oxo-4,5-dihydro-1,2,4-oxadiazol-3-yl)picolinamide, or the pharmaceutically-acceptable salt thereof, the tautomer thereof, the pharmaceutically-acceptable salt of the tautomer, the stereoisomer thereof, or the mixture thereof.

202. The compound of embodiment 113, wherein the compound is (S)-2-ethyl-4-((4-(3-fluoro-4-(trifluoromethoxy)phenyl)-3,4-dihydro-2H-pyrano[3,2-b]pyridin-4-yl)carbamoyl)benzoic acid, or the pharmaceutically-acceptable salt thereof, the tautomer thereof, the pharmaceutically-acceptable salt of the tautomer, the stereoisomer thereof, or the mixture thereof.

203. The compound of embodiment 113, wherein the compound is (S)-4-((4-(3-fluoro-4-(trifluoromethoxy)phenyl)-3,4-dihydro-2H-pyrano[3,2-b]pyridin-4-yl)carbamoyl)-3-methoxybenzoic acid, or the pharmaceutically-acceptable salt thereof, the tautomer thereof, the pharmaceutically-acceptable salt of the tautomer, the stereoisomer thereof, or the mixture thereof.

204. The compound of embodiment 113, wherein the compound is (S)-6-((4-(3-fluoro-4-(trifluoromethoxy)phenyl)-3,4-dihydro-2H-pyrano[3,2-b]pyridin-4-yl)carbamoyl)-1-methyl-1H-indole-2-carboxylic acid, or the pharmaceutically-acceptable salt thereof, the tautomer thereof, the pharmaceutically-acceptable salt of the tautomer, the stereoisomer thereof, or the mixture thereof.

205. The compound of embodiment 113, wherein the compound is (S)-2-amino-4-((4-(3-fluoro-4-(trifluoromethoxy)phenyl)-3,4-dihydro-2H-pyrano[3,2-b]pyridin-4-yl)carbamoyl)benzoic acid, or the pharmaceutically-acceptable salt thereof, the tautomer thereof, the pharmaceutically-acceptable salt of the tautomer, the stereoisomer thereof, or the mixture thereof.

206. The compound of embodiment 113, wherein the compound is (S)-4-((4-(3-fluoro-4-(trifluoromethoxy)phenyl)-3,4-dihydro-2H-pyrano[3,2-b]pyridin-4-yl)carbamoyl)-2-methylbenzoic acid, or the pharmaceutically-acceptable salt thereof, the tautomer thereof, the pharmaceutically-acceptable salt of the tautomer, the stereoisomer thereof, or the mixture thereof.

207. The compound or tautomer of any one of embodiments 113-206 in a neutral form.

208. The compound of any one of embodiments 113-206 in a neutral form.

209. The pharmaceutically-acceptably salt of the compound or the pharmaceutically acceptable salt of the tautomer of any one of embodiments 113-206.

210. The pharmaceutically-acceptably salt of the compound of any one of embodiments 113-206.

211. A pharmaceutical composition comprising the compound according to any one of embodiments 113-206 or the pharmaceutically-acceptable salt thereof, the tautomer thereof, the pharmaceutically-acceptable salt of the tautomer, the stereoisomer thereof, or the mixture thereof, and a pharmaceutically-acceptable diluent or carrier.

212. A method of treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders in a subject, the method comprising administering the compound according to any one of embodiments 113-206 or the pharmaceutically-acceptable salt thereof, the tautomer thereof, the pharmaceutically-acceptable salt of the tautomer, the stereoisomer thereof, or the mixture thereof to the subject.

213. The method of embodiment 212, wherein the subject is suffering from neuropathic pain.

214. The method of embodiment 212, wherein the subject is suffering from migraine pain.

215. The use of the compound according to any one of embodiments 113-206 or the pharmaceutically-acceptable salt thereof, the tautomer thereof, the pharmaceutically-acceptable salt of the tautomer, the stereoisomer thereof, or the mixture thereof in the preparation of a medicament.

216. The use of the compound according to any one of embodiments 113-206 or the pharmaceutically-acceptable salt thereof, the tautomer thereof, the pharmaceutically-acceptable salt of the tautomer, the stereoisomer thereof, or the mixture thereof for treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders in a subject.

217. The use of embodiment 216, wherein the use is for treating neuropathic pain.

218. The use of embodiment 216, wherein the use is for treating migraine.

219. The compound according to any one of embodiments 113-206 or the pharmaceutically-acceptable salt thereof, the tautomer thereof, the pharmaceutically-acceptable salt of the tautomer, the stereoisomer thereof, or the mixture thereof for treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders in a subject.

220. The compound of embodiment 219 or the pharmaceutically-acceptable salt thereof, the tautomer thereof, the pharmaceutically-acceptable salt of the tautomer, the stereoisomer thereof, or the mixture thereof for treating neuropathic pain in a subject.

221. The compound of embodiment 219 or the pharmaceutically-acceptable salt thereof, the tautomer thereof, the pharmaceutically-acceptable salt of the tautomer, the stereoisomer thereof, or the mixture thereof for treating migraine in a subject.

### EXAMPLES

Unless otherwise noted, all materials were obtained from commercial suppliers and used without further purification. All parts are by weight and temperatures are in degrees centigrade unless otherwise indicated. All microwave assisted reactions were conducted with a Smith Synthesizer from Biotage. Mass spectral data was determined by electrospray ionization technique. All examples were purified to >90% purity as determined by high-performance liquid chromatography. Unless otherwise stated, reactions were run at room temperature.

The following abbreviations are used:
DABCO—1,4-diazabicyclo[2.2.2]octane
DCM—dichloromethane
DIPEA—diisopropyl ethylamine
DMSO—dimethyl sulfoxide

### DMF—N,N-dimethylformamide

EDCI—1-ethyl-3-(3-dimethylaminopropyl) carbodiimide
Et₂O—diethyl ether
EtOAc—ethyl acetate
EtOH—ethyl alcohol
HATU—2-(7-Aza-1H-benzotriazole-1-yl)-1,1,3,3-tetramethyluronium hexafluorophosphate

MeCN—acetonitrile

MeOH—methyl alcohol
2-MeTHF—2-methyl tetrahydrofuran
n-BuLi—n-butyllithium
SFC—supercritical fluid chromatography
RBF—round bottom flask
T3P—2,4,6-Tripropyl-1,3,5,2,4,6-trioxatriphosphorinane-2,4,6-trioxide
TBAF—tetra-n-butylammonium fluoride
TEA—triethylamine
TFA—trifluoroacetic acid
THF—tetrahydrofuran
h—hour
min—min
rt—room temperature (22-25° C.)
mL milliliters
μL microliters
g grams
micrograms
mg milligrams
μmoL micromolar

### General Method of Preparation

The compounds described herein are prepared using techniques known to one skilled in the art through the reaction sequences depicted in Schemes 1-4 as well as by other methods. Furthermore, in the following schemes, where specific acids, bases, reagents, coupling agents, solvents, etc. are mentioned, it is understood that other suitable acids, bases, reagents, coupling agents, solvents, etc. may be used and are included within the scope of the present invention.

Amines used for the synthesis of compounds of the present invention were prepared as described in Scheme 1. Ketones of the Formula (1) were treated with aryl or heteroaryl metal halides of Formula (2) at low temperature to give alcohols of the Formula (3). The alcohol of Formula (4) was treated with substituted cyanides (4) and sulfuric acid to give amides of Formula (5). Amides of Formula (5) could be hydrolized with acid or base to give amines of Formula (6c).

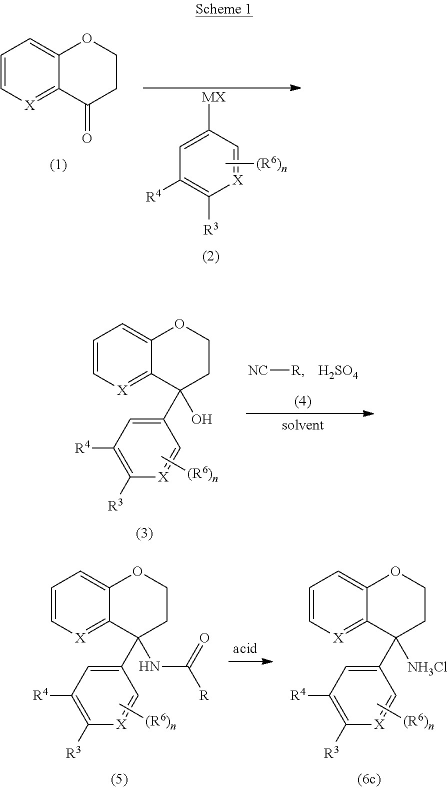

An alternative approach to amines of Formula (6c) is shown in Scheme 2. Ketones of the Formula (7) were treated with 2-methylpropane-2-sulfinamide and titanium ethoxide in 2-MeTHF to give sulfinimines of the Formula (8). The compounds of Formula (8) were treated with aryl or heteroaryl metal halides of Formula (9) at low temperature to give sulfinamides of the Formula (10). Hydrolysis of sulfinamides (10) with hydrochloric acid in MeOH gives diaryl amines of Formula (6c).

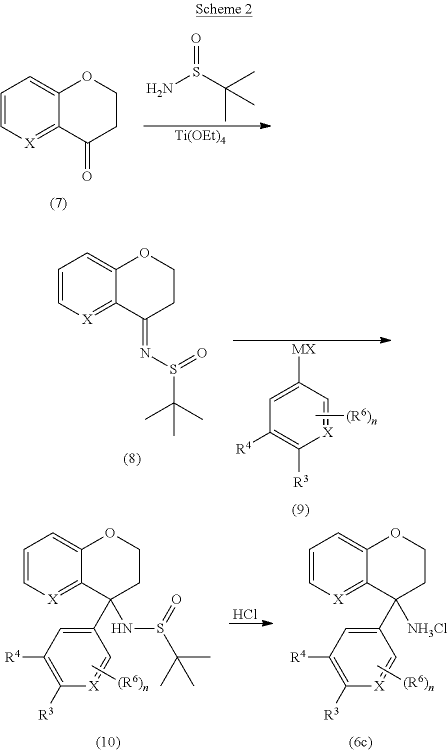

The method described in Scheme 2 can be adapted to a asymmetric syntheses using the appropriate (R)- or (S)-2-methylpropane-2-sulfinamides to give sulfinimines of the Formula (10a) or (10b). Subsequent aryl metal addition and hydrolysis provides chiral amines of Formula (6a) or (6b).

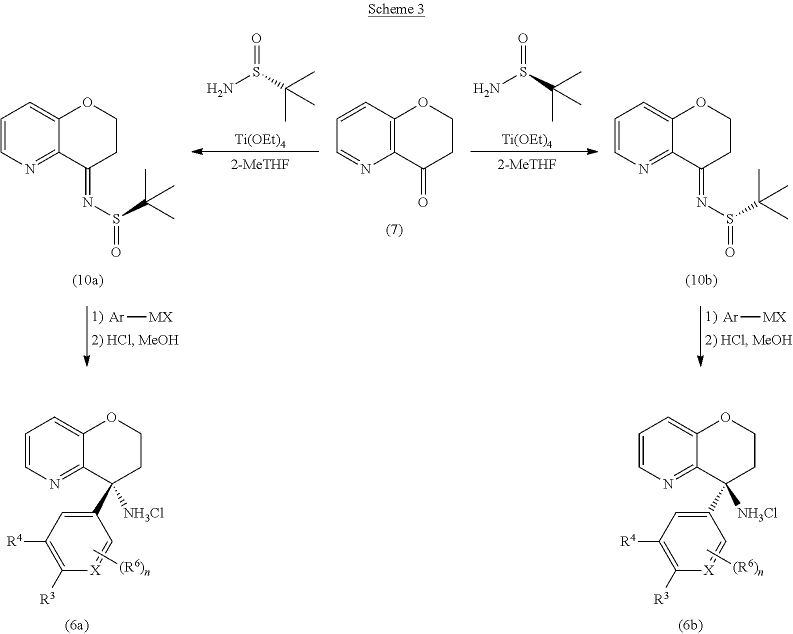

The coupling reaction of chromanamines of Formula (6a-c) with various carboxylic acids of Formula (11) provides amides of Formula (Ia), and the coupling can be performed as shown in Scheme 4. The coupling reaction can be mediated by a suitable coupling agent such as HATU in the presence of a base in a suitable solvent to afford compounds of the present invention. Alternatively, ureas, carbamates, or sulfonamides, can also be prepared utilizing the appropriate partner and coupling conditions.

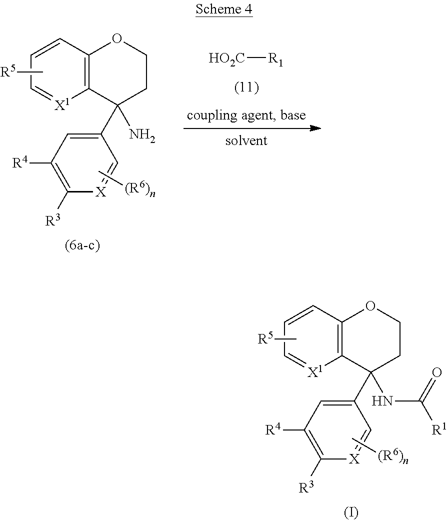

### Experimentals for Intermediates

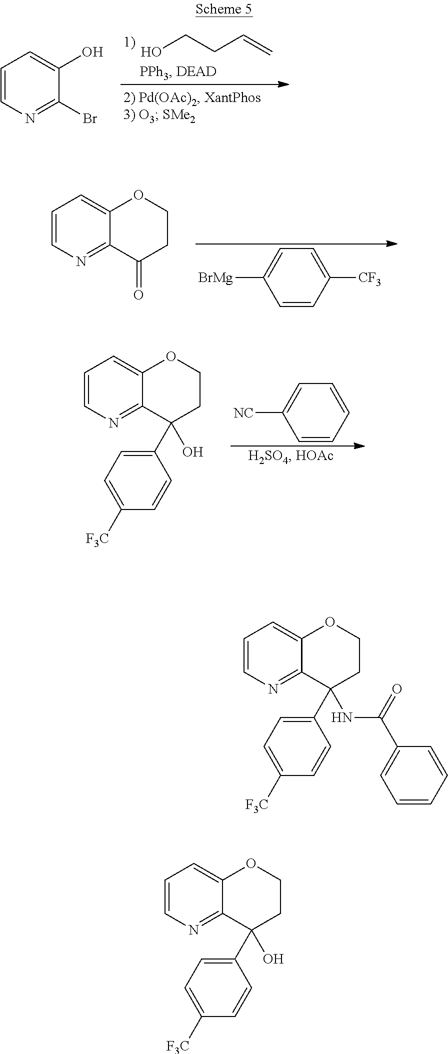

### Intermediate 1: 4-(4-(trifluoromethyl)phenyl)-3,4-dihydro-2H-pyrano[3,2-b]pyridin-4-ol

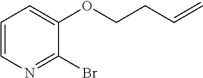

Step 1. 2-bromo-3-(but-3-en-1-yloxy)pyridine

Diethyl azodicarboxylate (95 mL, 0.6 mol) was added dropwise to a stirred mixture of 2-bromo-3-hydroxypyridine (97 g, 0.55 mol), 3-buten-1-ol (47.7 mL, 0.55 mol), and PPh₃ (175.3 g, 0.66 mol) in THF (970 mL) at 0° C. under a N₂ atmosphere. The reaction mixture was warmed to 50° C. in an oil bath and stirred for 17.5 h. Reaction progress was monitored by TLC (15% EtOAc in hexane, UV active). The reaction mixture was cooled to ambient temperature and diluted with saturated NaHCO₃ solution (500 mL). The aqueous solution was extracted with EtOAc (1 L). The organic layer was dried over Na₂SO₄ (200 g), and concentrated. The residual product was purified by column chromatography using 60-120 mesh, eluting with 5% EtOAc in hexane to afford the title compound as a pale yellow oil. ¹H NMR (400 MHz, CDCl₃): δ 7.97-7.98 (m, 1H), 7.13-7.22 (m, 1H), 7.11 (d, J=8 Hz, 1H), 5.89-5.99 (m, 1H), 5.13-5.23 (m, 2H), 4.06-4.11 (m, 2H), 2.59-2.64 (m, 2H).

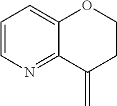

Step 2. 4-methylene-3,4-dihydro-2H-pyrano[3,2-b]pyridine

To a solution of 2-bromo-3-(but-3-en-1-yloxy)pyridine (130 g, 0.567 mol) in DMF (1.3 L), PPh₃ (59.5 g, 0.22 mol), Pd(OAc)₂ (19.9 g, 85 mol), KOAc (278.25 g, 2.835 mol), and tetraethyl ammonium chloride hydrate (187.9 g, 1.134 mol) were added under argon atmosphere. The flask was purged with argon for 15 min, and then the resulting reaction mixture was stirred at 110° C. for 16 h. Reaction progress was monitored by TLC (10% EtOAc in hexane, UV active). The reaction mixture was diluted with EtOAc and saturated NaHCO₃ solution. The organic layer was separated and dried over Na₂SO₄ (200 g), and concentrated. The product thus obtained was purified by column chromatography using 60-120 mesh, eluting with 5% EtOAc in hexane to afford the title compound as a pale yellow oil. MS (ESI pos. ion) m/z: 148 (MH+).

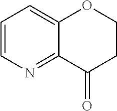 [SMILES: O=C1CCOc2cccnc21]

Step 3. 2H-pyrano[3,2-b]pyridin-4(3H)-one

To a solution of 4-methylene-3,4-dihydro-2H-pyrano[3,2-b]pyridine (175 g, 1.19 mol) in a mixture of solvents (MeOH:CHCl₃) was added a catalytic amount of NaHCO₃ (1 g). The reaction mixture was cooled to −78° C. and purged with O₃. Reaction progress was monitored by TLC (50% EtOAc in hexane, UV active). After 16 h, the reaction mixture was quenched with dimethyl sulfide (50 mL) at −78° C. The resulting mixture was stirred for 12 h at ambient temperature. The reaction mixture was then diluted with EtOAc and water. The organic layer was washed with water (3×500 mL), dried over Na₂SO₄ (200 g), and concentrated under reduced pressure. The residue thus obtained was recrystallized with diethyl ether to give the title compound as a colorless solid. MS (ESI pos. ion) m/z: 150.2 (MH+).

### Grignard Procedure A:

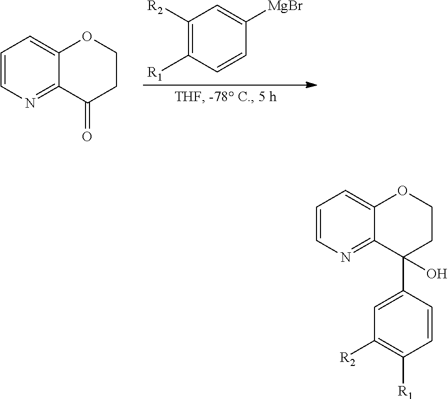

To a stirred suspension of magnesium (402 mmol) in THF (10 times), was added the corresponding aryl bromide (201 mmol). The reaction was stirred for 5 h (cautious: slightly exothermic, cooled with water bath if needed). The Grignard solution was cannulated into a stirred solution of the 2H-pyrano[3,2-b]pyridin-4(3H)-one (67.1 mmol) in THF (10 times) at −78° C. in a drop wise fashion. The stirring was continued for 1 h. The reaction mixture was quenched with saturated NH₄Cl (250 mL), and the product was extracted with EtOAc (2×500 mL). The combined organic layers were dried over Na₂SO₄, concentrated, and purified by column chromatography using silica (100-200 mesh silica) with 25-30% EtOAc in petroleum ether as eluent to give the desired alcohol.

### Grignard Procedure B:

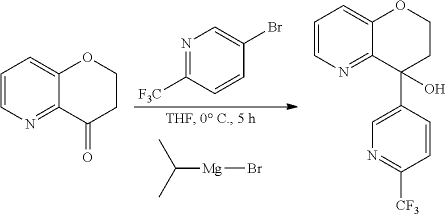

To a 0° C. solution of 5-bromo-2-(trifluoromethyl)pyridine (0.604 g, 2.68 mmol) in THF (10 mL) was added isopropyl magnesium bromide (1 mL, 2.68 mmol) in a drop-wise fashion. The reaction mixture was stirred in a cooling bath for 1 h and was then treated with 2H-pyrano[3,2-b]pyridin-4(3H)-one (200 mg, 1.34 mmol) in THF (5 mL). After stirring for 1 h, saturated NH₄Cl (50 mL) was added to the reaction mixture, and the aqueous solution was extracted with EtOAc (3×10 mL). The combined organic layers were dried over Na₂SO₄, and concentrated. The residue was purified by column chromatography to give the desired alchohol.

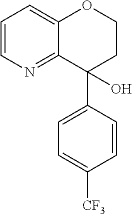 [SMILES: OC1(c2ccc(C(F)(F)F)cc2)CCOc2cccnc21]

### Intermediate 1: 4-(4-(trifluoromethyl)phenyl)-3,4-dihydro-2H-pyrano[3,2-b]pyridin-4-ol

MS (ESI pos. ion) m/z: 295.9 (MH+); ¹H NMR (400 MHz, CDCl₃): δ 8.2 (d, J=3.6 Hz, 1H), 7.6 (d, J=8.0 Hz, 2H), 7.4 (d, J=8.0 Hz, 2H) 7.3 (d, J=6.4 Hz, 1H), 7.2 (m, 1H), 4.4 (m, 1H), 4.3 (m, 1H), 4.0 (s, 1H), 2.5 (m, 1H), 2.3 (m, 1H).

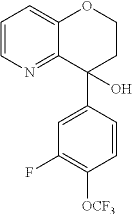 [SMILES: OC1(c2ccc(OC(F)(F)F)c(F)c2)CCOc2cccnc21]

### Intermediate 2: 4-(3-fluoro-4-(trifluoromethoxy)phenyl)-3,4-dihydro-2H-pyrano[3,2-b]pyridin-4-ol

MS (ESI pos. ion) m/z: 330.2 (MH+); ¹H NMR (300 MHz, DMSO-d₆): δ 8.06 (dd, J=1.5, 2.7 Hz, 1H), 7.40-7.49 (m, 2H), 7.23-7.33 (m, 2H), 7.12 (d, J=8.7 Hz, 1H), 6.34 (s, 1H), 4.24-4.41 (m, 2H), 2.35-2.45 (m, 1H), 2.09-2.15 (m, 1H).

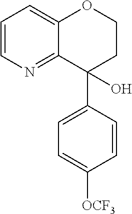 [SMILES: OC1(c2ccc(OC(F)(F)F)cc2)CCOc2cccnc21]

### Intermediate 3: 4-(4-(trifluoromethoxy)phenyl)-3,4-dihydro-2H-pyrano[3,2-b]pyridin-4-ol

MS (ESI pos. ion) m/z: 312.0 (MH+); ¹H NMR (400 MHz, CDCl₃): δ 8.2 (d, J=3.6 Hz, 1H), 7.6 (d, J=8.0 Hz, 2H), 7.4 (d, J=8.0 Hz, 2H) 7.3 (d, J=6.4 Hz, 1H), 7.2 (m, 1H), 4.4 (m, 1H), 4.3 (m, 1H), 4.0 (s, 1H), 2.5 (m, 1H), 2.3 (m, 1H).

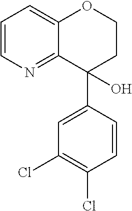 [SMILES: OC1(c2ccc(Cl)c(Cl)c2)CCOc2cccnc21]

### Intermediate 4: 4-(3,4-dichlorophenyl)-3,4-dihydro-2H-pyrano[3,2-b]pyridin-4-ol

MS (ESI pos. ion) m/z: 296.1 (MH+); ¹H NMR (400 MHz, CDCl₃): δ 8.2 (q, J=1.2, 4.0 Hz, 1H), 7.4 (m, 2H), 7.2 (m, 2H) 7.0 (dd, J=2.4, 8.4 Hz, 1H), 4.3 (m, 1H), 4.1 (m, 2H), 3.9 (s, 1H), 2.5 (m, 1H), 2.3 (m, 1H).

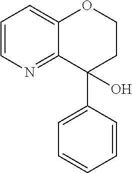 [SMILES: OC1(c2ccccc2)CCOc2cccnc21]

### Intermediate 5: 4-phenyl-3,4-dihydro-2H-pyrano[3,2-b]pyridin-4-ol

MS (ESI pos. ion) m/z: 228.1 (MH+); ¹H NMR (300 MHz, CDCl₃): δ 8.20-8.18 (m, 1H), 7.34-7.16 (m, 6H), 4.35-4.28 (m, 1H), 4.15-4.03 (m, 1H), 2.55-2.46 (m, 1H), 2.40-2.33 (m, 1H).

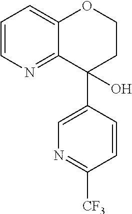 [SMILES: OC1(c2ccc(C(F)(F)F)nc2)CCOc2cccnc21]

### Intermediate 6: 4-(6-(trifluoromethyl)pyridin-3-yl)-3,4-dihydro-2H-pyrano[3,2-b]pyridin-4-ol

MS (ESI pos. ion) m/z: 297.0 (MH+).

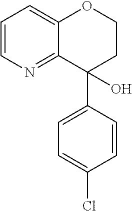 [SMILES: OC1(c2ccc(Cl)cc2)CCOc2cccnc21]

### Intermediate 7: 4-(4-chlorophenyl)-3,4-dihydro-2H-pyrano[3,2-b]pyridin-4-ol

MS (ESI pos. ion) m/z: 262.1 (MH+). ¹H NMR (DMSO-d₆) δ: 8.05 (dd, J=4.3, 1.4 Hz, 1H), 7.30-7.36 (m, 2H), 7.19-7.30 (m, 4H), 6.09 (s, 1H), 4.35 (td, J=11.0, 2.3 Hz, 1H), 4.21 (dt, J=11.0, 4.1 Hz, 1H), 2.31 (ddd, J=14.3, 10.9, 3.7 Hz, 1H), 2.05-2.18 (m, 1H).

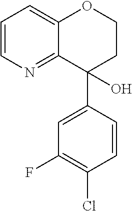 [SMILES: OC1(c2ccc(Cl)c(F)c2)CCOc2cccnc21]

### Intermediate 8: 4-(4-chloro-3-fluorophenyl)-3,4-dihydro-2H-pyrano[3,2-b]pyridin-4-ol

MS (ESI pos. ion) m/z: 280.0 (MH+). ¹H NMR (DMSO-d₆) δ: 8.07 (dd, J=4.3, 1.4 Hz, 1H), 7.49 (t, J=8.1 Hz, 1H), 7.35 (dd, J=11.0, 2.0 Hz, 1H), 7.31 (dd, J=8.3, 1.5 Hz, 1H), 7.25 (dd, J=8.2, 4.3 Hz, 1H), 7.06 (dd, J=8.4, 1.6 Hz, 1H), 6.27 (s, 1H), 4.38 (td, J=11.2, 2.2 Hz, 1H), 4.28 (dt, J=11.1, 3.9 Hz, 1H), 2.33-2.46 (m, 1H), 2.06-2.20 (m, 1H).

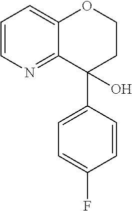 [SMILES: OC1(c2ccc(F)cc2)CCOc2cccnc21]

### Intermediate 9: 4-(4-fluorophenyl)-3,4-dihydro-2H-pyrano[3,2-b]pyridin-4-ol

MS (ESI pos. ion) m/z: 246.2 (MH+). ¹H NMR (DMSO-d₆) δ: 8.06 (dd, J=4.3, 1.4 Hz, 1H), 7.18-7.33 (m, 4H), 7.04-7.13 (m, 2H), 6.03 (s, 1H), 4.34 (td, J=8, 2.4 Hz, 1H), 4.19 (dt, J=11.1, 4.2 Hz, 1H), 2.24-2.38 (m, 1H), 2.07-2.18 (m, 1H).

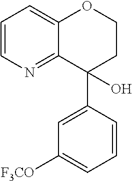 [SMILES: OC1(c2cccc(OC(F)(F)F)c2)CCOc2cccnc21]

### Intermediate 10: 4-(3-(trifluoromethoxy)phenyl)-3,4-dihydro-2H-pyrano[3,2-b]pyridin-4-ol

MS (ESI pos. ion) m/z: 312.0 (MH+).

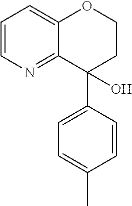 [SMILES: Cc1ccc(C2(O)CCOc3cccnc32)cc1]

### Intermediate 11: 4-(p-tolyl)-3,4-dihydro-2H-pyrano[3,2-b]pyridin-4-ol

MS (ESI pos. ion) m/z: 242.1 (MH+).

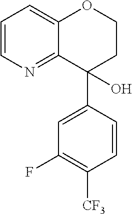 [SMILES: OC1(c2ccc(C(F)(F)F)c(F)c2)CCOc2cccnc21]

### Intermediate 12: 4-(3-fluoro-4-(trifluoromethyl)phenyl)-3,4-dihydro-2H-pyrano[3,2-b]pyridin-4-ol

MS (ESI pos. ion) m/z: 314.1 (MH+). ¹H NMR (DMSO-d₆) δ: 8.05 (dd, J=4.3, 1.4 Hz, 1H), 7.69 (t, J=8.0 Hz, 1H), 7.43 (d, J=12.5 Hz, 1H), 7.28-7.36 (m, 1H), 7.20-7.28 (m, 2H), 6.41 (s, 1H), 4.25-4.44 (m, 2H), 2.36-2.47 (m, 1H), 2.12 (dt, J=14.3, 2.5 Hz, 1H).

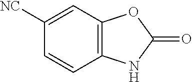 [SMILES: N#Cc1ccc2[nH]c(=O)oc2c1]

### Intermediate 13: 2-oxo-2,3-dihydrobenzo[d]oxazole-6-carbonitrile

To a solution of 6-bromo benzoxazolinone (2 g, 9.4 mmol) in DMF (20 mL) was added CuCN (16.79 g, 188 mmol), and the reaction was stirred at 175° C. for 6 h. The reaction progress was monitored by TLC (100% EtOAc). The reaction was diluted with EtOAc (10 mL) and filtered through Celite® brand filter agent. The organic layer was concentrated and purified by column chromatography (silica gel, 0-50% EtOAc in hexanes) to give the title compound. MS (ESI pos. ion) m/z: 158.9 (MH−).

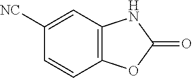 [SMILES: N#Cc1ccc2oc(=O)[nH]c2c1]

### Intermediate 14: 2-oxo-2,3-dihydrobenzo[d]oxazole-5-carbonitrile

To a solution of 5-bromo benzoxazolinone (1 g, 4.7 mmol) in DMF (15 mL), were added Zn(CN)₂ (1 g, 9.4 mmol) and Pd(PPh₃)₄. The reaction was stirred at 150° C. for 24 h. The reaction progress was monitored by TLC (100% EtOAc). The reaction was diluted with EtOAc (10 mL) and filtered through Celite® brand filter agent. The organic layer was concentrated and purified by column chromatography (silca gel, 0-50% EtOAc in hexanes) to give the title compound. ¹H NMR (400 MHz, CDCl₃): δ 8.6 (br s, 1H), 7.5 (dd, J=1.8, 8.4 Hz, 1H), 7.3-7.4 (m, 2H).

### Ritter Reaction Procedure a:

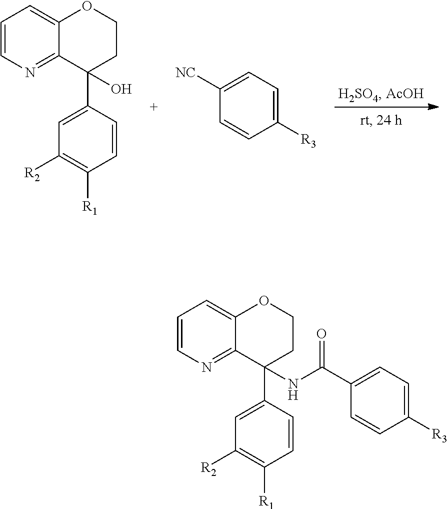

To a stirred mixture of 4-(substituted) phenyl)-3,4-dihydro-2H-pyrano[3,2-b]pyridin-4-ol (1.5 mmol) and 4-substituted benzonitrile (9.1 mmol) at 0° C. were added acetic acid (23.3 mmol) and conc. H₂SO₄ (15.9 mmol). The ice bath was removed, and the reaction was stirred at ambient temperature for 24 h. The reaction mixture was diluted with water (20 mL) and EtOAc (40 mL) and quenched slowly with saturated NaHCO₃ solution (125 mL). The aqueous layer was subsequently extracted with EtOAc (2×50 mL). The combined organic layers were washed with brine (50 mL), dried over Na₂SO₄, concentrated, and purified by column chromatography to give the desired product.

### Ritter Reaction Procedure B:

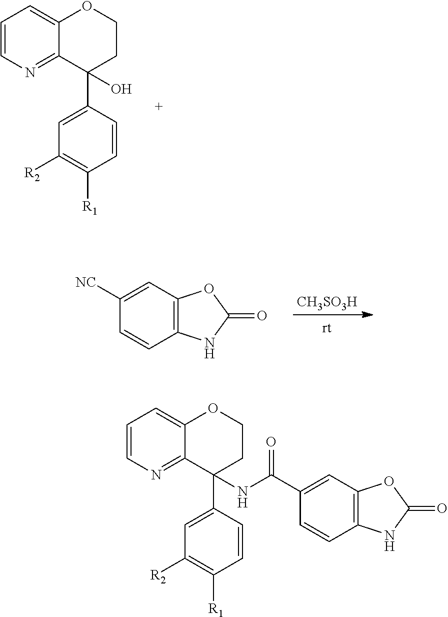

To a stirred mixture of 4-(substituted) phenyl)-3,4-dihydro-2H-pyrano[3,2-b]pyridin-4-ol (1.5 mmol) and 2-oxo-2,3-dihydrobenzo[d]oxazole-6-carbonitrile (1.5 mmol) at 0° C. was added methanesulfonic acid (10 mL). The ice bath was removed, and the reaction was stirred at ambient temperature for 24 h. The reaction mixture was diluted with water (20 mL) and EtOAc (40 mL) and quenched slowly with saturated NaHCO₃ solution (125 mL). The organic layer was separated, and the aqueous layer extracted with EtOAc (2×50 mL). The combined organic layers were washed with brine (50 mL), dried over Na₂SO₄, concentrated, and purified by column chromatography to give the desired products.

### Ritter Reaction Procedure C:

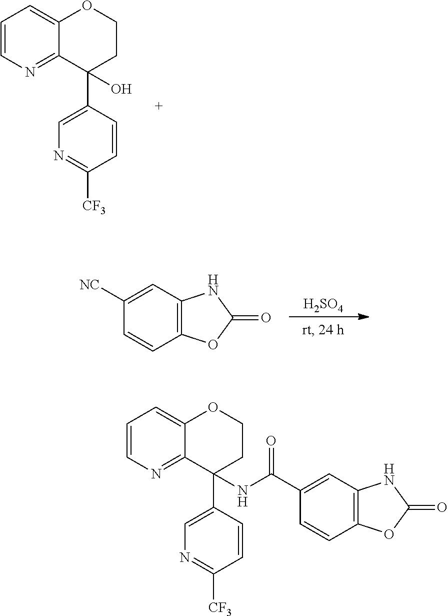

To a cooled mixture at 0° C. of 4-(6-(trifluoromethyl)pyridin-3-yl)-3,4-dihydro-2H-pyrano[3,2-b]pyridin-4-ol (1 g, 3 mmol) and 2-oxo-2,3-dihydrobenzo-[d]oxazole-5-carbonitrile (810 mg, 5 mmol) was added conc. H₂SO₄ (20 mL). The ice bath was removed and the reaction was stirred at ambient temperature for 24 h. The reaction mixture was diluted with water (20 mL) and EtOAc (40 mL), then slowly quenched with saturated NaHCO₃ solution (125 mL). The organic layer was separated and the aqueous layer extracted with EtOAc (2×50 mL). The combined organic layers were washed with brine (50 mL), dried over Na₂SO₄, concentrated, and purified by column chromatography to give the desired product.

TABLE 1A
Reaction conditions used to prepare Examples 1-27.

| | Alcohol | Grignard | Ritter | Nitrite Inter- |
|---|---|---|---|---|
| Ex. # | Intermediate | procedure | procedure | mediate |
| 1¹ | 1 | A | A | 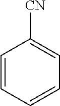 |
| 2² | 1 | A | A | 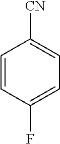 |
| 3³ | 4 | A | A | 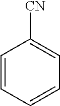 |
| 4⁴ | 1 | A | A | 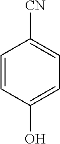 |
| 5⁵ | 1 | A | A | 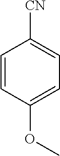 |
| 6 | 2 | A | A | 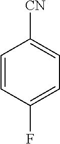 |
| 7 | 2 | A | A | 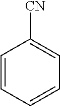 |
| 8 | 3 | A | A | 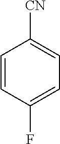 |
| 9 | 3 | A | A | 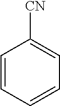 |
| 10 | 4 | A | A | 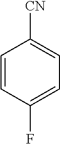 |
| 11 | 5 | A | A | 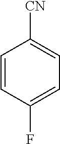 |
| 12 | 5 | A | A | 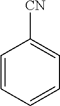 |
| 13 | 6 | B | A | 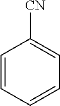 |
| 14⁶ | 7 | A | A | 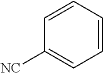 |
| 15⁷ | 8 | A | A | 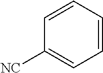 |
| 16⁸ | 9 | A | A | 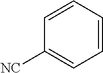 |
| 17⁹ | 10 | A | A | 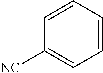 |
| 18¹⁰ | 11 | A | A | 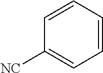 |
| 19¹¹ | 12 | A | A | 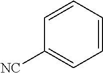 |
| 20¹² | 1 | A | B | 12 |
| 21¹³ | 1 | A | B | 12 |
| 22 | 3 | A | B | 7 |
| 23 | 3 | A | B | 8 |
| 24 | 4 | A | B | 7 |
| 25 | 4 | A | B | 8 |
| 26 | 5 | A | A | 7 |
| 27 | 6 | B | C | 8 |

¹Purified via preparative SFC [using a AD-H (2 × 15 cm); mobile phase: 20% methanol (0.1% NH₄OH)/liquid CO₂, at a flow rate of 70 mL/min] resulting in Peak 1 and 2 fractions with enantiomeric excess >99%. Peak 2 correlates with the (S) enantiomer.
²Purified via preparative SFC [using a Chiralpak ADH(21 × 250 mm, 5 um); mobile phase: 20% methanol (40 mM NH₃)/liquid CO₂, at a flow rate of 70 mL/min] resulting in Peak 1 and 2 fractions with enantiomeric excess >99%. Peak 2 correlates with the (S) enantiomer.
³Purified via preparative SFC [using a AD-H (2 × 15 cm); mobile phase: 20% methanol/liquid CO₂, at a flow rate of 60 mL/min] resulting in Peak 1 and 2 fractions with enantiomeric excess >99%. Peak 2 correlates with the (S) enantiomer.
⁴Purified via preparative SFC [using a AD-H (21 × 250 mm, 5 um); mobile phase: 12% methanol (40 mM NH₃)/liquid CO₂, at a flow rate of 70 mL/min] resulting in Peak 1 and 2 fractions with enantiomeric excess >99%. Peak 2 correlates with the (S) enantiomer.
⁵Purified via preparative SFC [using a Chiralcel OJH (21 × 250 mm, 5 um); mobile phase: 20% ethanol (20 mM NH₃)/liquid CO₂, at a flow rate of 75 mL/min] resulting in Peak 1 and 2 fractions with enantiomeric excess >98%. Peak 2 correlates with the (S) enantiomer.
⁶Purified via preparative SFC [using a AD-H column (250 × 20 mm); mobile phase: 20% methanol (20 mM NH₃)/liquid CO₂, at a flow rate of 75 mL/min] resulting in Peak 1 and 2 fractions with enantiomeric excess >98%. Peak 2 correlates with the (S) enantiomer.
⁷Purified via preparative SFC [using a AD-H column (250 × 20 mm); mobile phase: 20% methanol (20 mM NH₃)/liquid CO₂, at a flow rate of 75 mL/min] resulting in Peak 1 and 2 fractions with enantiomeric excess >98%. Peak 2 correlates with the (S) enantiomer.
⁸Purified via preparative SFC [using a AD-H column (250 × 20 mm); mobile phase: 20% methanol (20 mM NH₃)/liquid CO₂, at a flow rate of 75 mL/min] resulting in Peak 1 and 2 fractions with enantiomeric excess >98%. Peak 2 correlates with the (S) enantiomer.
⁹Purified via preparative SFC [using a AD-H column (250 × 20 mm); mobile phase: 10% methanol (20 mM NH₃)/liquid CO₂, at a flow rate of 75 mL/min] resulting in Peak 1 and 2 fractions with enantiomeric excess >98%. Peak 2 correlates with the (S) enantiomer.
¹⁰Purified via preparative SFC [using a AD-H column (250 × 20 mm); mobile phase: 20% methanol (20 mM NH₃)/liquid CO₂, at a flow rate of 75 mL/min] resulting in Peak 1 and 2 fractions with enantiomeric excess >98%. Peak 2 correlates with the (S) enantiomer.
¹¹Purified via preparative SFC [using a ODH column (21 × 250 mm, 5 um); mobile phase: 30% methanol (20 mM NH₃)/liquid CO₂, at a flow rate of 65 mL/min] resulting in Peak 1 and 2 fractions with enantiomeric excess >98%. Peak 2 correlates with the (S) enantiomer.
¹²Purified via preparative SFC [using a AD-H column (250 × 20 mm); mobile phase: 20% methanol/liquid CO₂, at a flow rate of 75 mL/min] resulting in Peak 1 and 2 fractions with enantiomeric excess >99%. Peak 2 correlates with the (S) enantiomer.
¹³Purified via preparative SFC [using a Chiralcel OJH (21 × 250 mm, 5 um); mobile phase: 20% ethanol (20 mM NH₃)/liquid CO₂, at a flow rate of 70 mL/min] resulting in Peak 1 and 2 fractions with enantiomeric excess >99%. Peak 2 correlates with the (S) enantiomer.

TABLE 1B
Examples 1-27 were prepared in a manner analagous to that
shown in Scheme 5 and described above.

| | | | MS |
|---|---|---|---|
| Ex. # | Structure | Compound Name | MH+ |
| 1¹ | 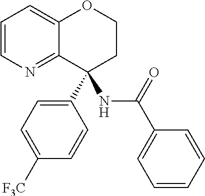 [SMILES: O=C(N[C@]1(c2ccc(C(F)(F)F)cc2)CCOc2cccnc21)c1ccccc1] | (S)-N-(4-(4- (trifluoromethyl)phenyl)- 3,4-dihydro-2H- pyrano[3,2-b]pyridin-4- yl)benzamide | 398.9 |
| 2² | 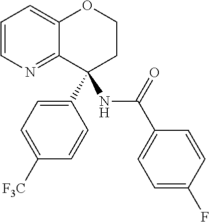 [SMILES: O=C(N[C@]1(c2ccc(C(F)(F)F)cc2)CCOc2cccnc21)c1ccc(F)cc1] | (S)-4-fluoro-N-(4-(4- (trifluoromethyl)phenyl)- 3,4-dihydro-2H- pyrano[3,2-b]pyridin-4- yl)benzamide | 416.7 |
| 3³ | 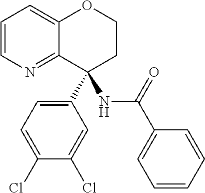 [SMILES: O=C(N[C@]1(c2ccc(Cl)c(Cl)c2)CCOc2cccnc21)c1ccccc1] | (S)-N-(4-(3,4- dichlorophenyl)-3,4- dihydro-2H-pyrano[3,2- b]pyridin-4-yl)benzamide | 399.0 |
| 4⁴ | 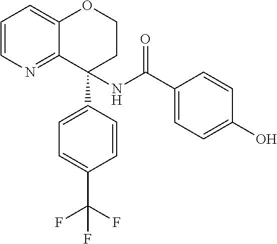 [SMILES: O=C(N[C@]1(c2ccc(C(F)(F)F)cc2)CCOc2cccnc21)c1ccc(O)cc1] | (S)-4-hydroxy-N-(4-(4- (trifluoromethyl)phenyl)- 3,4-dihydro-2H- pyrano[3,2-b]pyridin-4- yl)benzamide | 415.0 |
| 5⁵ | 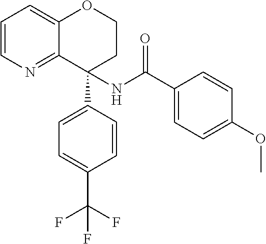 [SMILES: COc1ccc(C(=O)N[C@]2(c3ccc(C(F)(F)F)cc3)CCOc3cccnc32)cc1] | (S)-4-methoxy-N-(4-(4- (trifluoromethyl)phenyl)- 3,4-dihydro-2H- pyrano[3,2-b]pyridin-4- yl)benzamide | 429.2 |
| 6 | 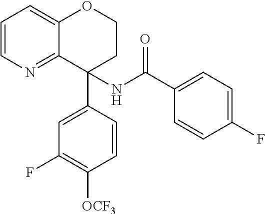 [SMILES: O=C(NC1(c2ccc(OC(F)(F)F)c(F)c2)CCOc2cccnc21)c1ccc(F)cc1] | 4-fluoro-N-(4-(3-fluoro-4- (trifluoromethoxy)phenyl)- 3,4-dihydro-2H- pyrano[3,2-b]pyridin-4- yl)benzamide | 451.3 |
| 7 | 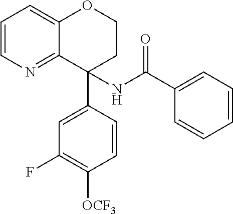 [SMILES: O=C(NC1(c2ccc(OC(F)(F)F)c(F)c2)CCOc2cccnc21)c1ccccc1] | N-(4-(3-fluoro-4- (trifluoromethoxy)phenyl)- 3,4-dihydro-2H- pyrano[3,2-b]pyridin-4- yl)benzamide | 433.0 |
| 8 | 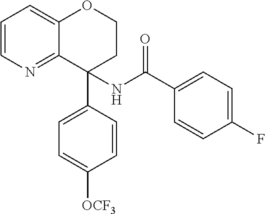 [SMILES: O=C(NC1(c2ccc(OC(F)(F)F)cc2)CCOc2cccnc21)c1ccc(F)cc1] | 4-fluoro-N-(4-(4- (trifluoromethoxy)phenyl)- 3,4-dihydro-2H- pyrano[3,2-b]pyridin-4- yl)benzamide | 433.1 |
| 9 | 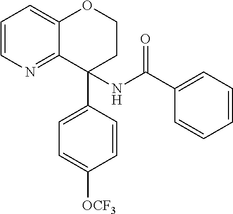 [SMILES: O=C(NC1(c2ccc(OC(F)(F)F)cc2)CCOc2cccnc21)c1ccccc1] | N-(4-(4- (trifluoromethoxy)phenyl)- 3,4-dihydro-2H- pyrano[3,2-b]pyridin-4- yl)benzamide | 415.1 |
| 10 | 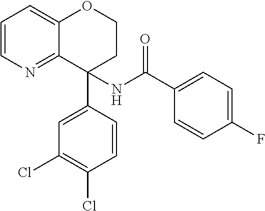 [SMILES: O=C(NC1(c2ccc(Cl)c(Cl)c2)CCOc2cccnc21)c1ccc(F)cc1] | N-(4-(3,4- dichlorophenyl)-3,4- dihydro-2H-pyrano[3,2- b]pyridin-4-yl)-4- fluorobenzamide | 416.9 |
| 11 | 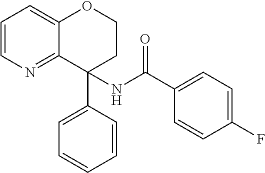 [SMILES: O=C(NC1(c2ccccc2)CCOc2cccnc21)c1ccc(F)cc1] | 4-fluoro-N-(4-phenyl-3,4- dihydro-2H-pyrano[3,2- b]pyridin-4-yl)benzamide | 348.9 |
| 12 | 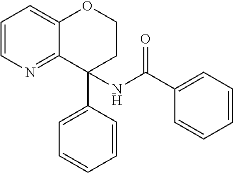 [SMILES: O=C(NC1(c2ccccc2)CCOc2cccnc21)c1ccccc1] | N-(4-phenyl-3,4-dihydro- 2H-pyrano[3,2-b]pyridin- 4-yl)benzamide | 331.0 |
| 13 | 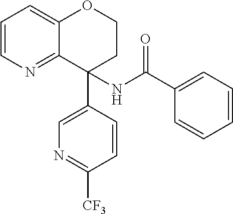 [SMILES: O=C(NC1(c2ccc(C(F)(F)F)nc2)CCOc2cccnc21)c1ccccc1] | N-(4-(6- (trifluoromethyl)pyridin- 3-yl)-3,4-dihydro-2H- pyrano[3,2-b]pyridin-4- yl)benzamide | 400.3 |
| 14⁶ | 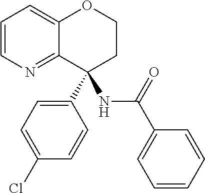 [SMILES: O=C(N[C@]1(c2ccc(Cl)cc2)CCOc2cccnc21)c1ccccc1] | (S)-N-(4-(4- chlorophenyl)-3,4- dihydro-2H-pyrano[3,2- b]pyridin-4-yl)benzamide | 365.1 |
| 15⁷ | 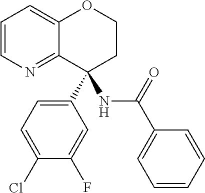 [SMILES: O=C(N[C@]1(c2ccc(Cl)c(F)c2)CCOc2cccnc21)c1ccccc1] | (S)-N-(4-(4-chloro-3- fluorophenyl)-3,4- dihydro-2H-pyrano[3,2- b]pyridin-4-yl)benzamide | 383.1 |
| 16⁸ | 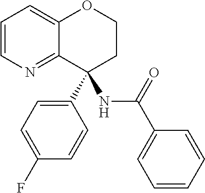 [SMILES: O=C(N[C@]1(c2ccc(F)cc2)CCOc2cccnc21)c1ccccc1] | (S)-N-(4-(4- fluorophenyl)-3,4- dihydro-2H-pyrano[3,2- b]pyridin-4-yl)benzamide | 349.1 |
| 17⁹ | 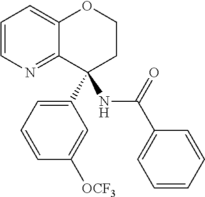 [SMILES: O=C(N[C@]1(c2cccc(OC(F)(F)F)c2)CCOc2cccnc21)c1ccccc1] | (S)-N-(4-(3- (trifluoromethoxy)phenyl)- 3,4-dihydro-2H- pyrano[3,2-b]pyridin-4- yl)benzamide | 415.2 |
| 18¹⁰ | 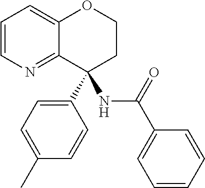 [SMILES: Cc1ccc([C@@]2(NC(=O)c3ccccc3)CCOc3cccnc32)cc1] | (S)-N-(4-(p-tolyl)-3,4- dihydro-2H-pyrano[3,2- b]pyridin-4-yl)benzamide | 345.2 |
| 19¹¹ | 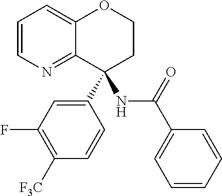 [SMILES: O=C(N[C@]1(c2ccc(C(F)(F)F)c(F)c2)CCOc2cccnc21)c1ccccc1] | (S)-N-(4-(3-fluoro-4- (trifluoromethyl)phenyl)- 3,4-dihydro-2H- pyrano[3,2-b]pyridin-4- yl)benzamide | 417.0 |
| 20¹² | 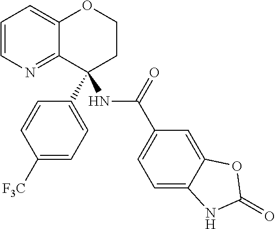 [SMILES: O=C(N[C@]1(c2ccc(C(F)(F)F)cc2)CCOc2cccnc21)c1ccc2[nH]c(=O)oc2c1] | (S)-2-oxo-N-(4-(4- (trifluoromethyl)phenyl)- 3,4-dihydro-2H- pyrano[3,2-b]pyridin-4- yl)-2,3- dihydrobenzo[d]oxazole- 6-carboxamide | 456.0 |
| 21¹³ | 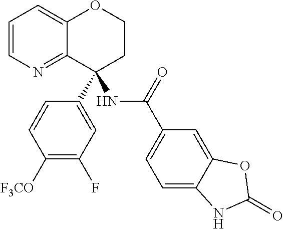 [SMILES: O=C(N[C@]1(c2ccc(OC(F)(F)F)c(F)c2)CCOc2cccnc21)c1ccc2[nH]c(=O)oc2c1] | (S)-N-(4-(3-fluoro-4- (trifluoromethoxy)phenyl)- 3,4-dihydro-2H- pyrano[3,2-b]pyridin-4- yl)-2-oxo-2,3- dihydrobenzo[d]oxazole- 6-carboxamide | 490.0 |
| 22 | 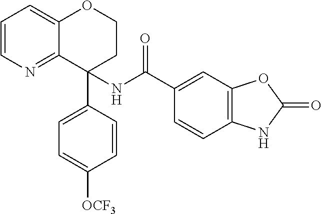 [SMILES: O=C(NC1(c2ccc(OC(F)(F)F)cc2)CCOc2cccnc21)c1ccc2[nH]c(=O)oc2c1] | 2-oxo-N-(4-(4- (trifluoromethoxy)phenyl)- 3,4-dihydro-2H- pyrano[3,2-b]pyridin-4- yl)-2,3- dihydrobenzo[d]oxazole- 6-carboxamide | 471.9 |
| 23 | 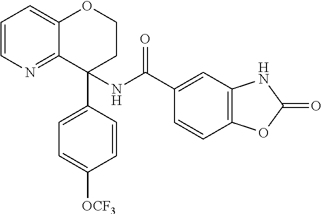 [SMILES: O=C(NC1(c2ccc(OC(F)(F)F)cc2)CCOc2cccnc21)c1ccc2oc(=O)[nH]c2c1] | 2-oxo-N-(4-(4- (trifluoromethoxy)phenyl)- 3,4-dihydro-2H- pyrano[3,2-b]pyridin-4- yl)-2,3- dihydrobenzo[d]oxazole- 5-carboxamide | 472.0 |
| 24 | 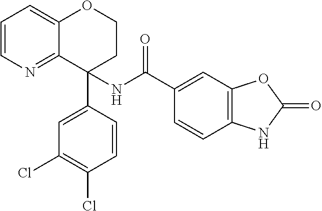 [SMILES: O=C(NC1(c2ccc(Cl)c(Cl)c2)CCOc2cccnc21)c1ccc2[nH]c(=O)oc2c1] | N-(4-(3,4- dichlorophenyl)-3,4- dihydro-2H-pyrano[3,2- b]pyridin-4-yl)-2-oxo-2,3- dihydrobenzo[d]oxazole- 6-carboxamide | 456.0 |
| 25 | 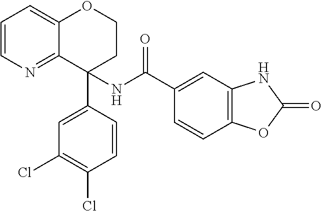 [SMILES: O=C(NC1(c2ccc(Cl)c(Cl)c2)CCOc2cccnc21)c1ccc2oc(=O)[nH]c2c1] | N-(4-(3,4- dichlorophenyl)-3,4- dihydro-2H-pyrano[3,2- b]pyridin-4-yl)-2-oxo-2,3- dihydrobenzo[d]oxazole- 5-carboxamide | 456.0 |
| 26 | 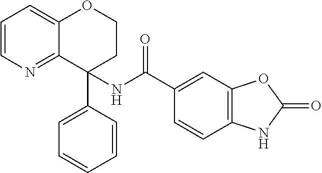 [SMILES: O=C(NC1(c2ccccc2)CCOc2cccnc21)c1ccc2[nH]c(=O)oc2c1] | 2-oxo-N-(4-phenyl-3,4- dihydro-2H-pyrano[3,2- b]pyridin-4-yl)-2,3- dihydrobenzo[d]oxazole- 6-carboxamide | 388.3 |
| 27 | 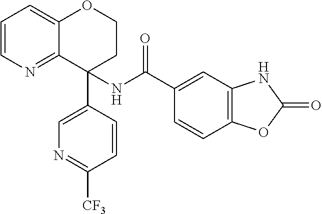 [SMILES: O=C(NC1(c2ccc(C(F)(F)F)nc2)CCOc2cccnc21)c1ccc2oc(=O)[nH]c2c1] | 2-oxo-N-(4-(6- (trifluoromethyl)pyridin- 3-yl)-3,4-dihydro-2H- pyrano[3,2-b]pyridin-4- yl)-2,3- dihydrobenzo[d]oxazole- 5-carboxamide | 457.1 |

¹Purified via preparative SFC [using a AD-H (2 × 15 cm); mobile phase: 20% methanol (0.1% NH₄OH)/liquid CO₂, at a flow rate of 70 mL/min] resulting in Peak 1 and 2 fractions with enantiomeric excess >99%. Peak 2 correlates with the (S) enantiomer.
²Purified via preparative SFC [using a Chiralpak ADH(21 × 250 mm, 5 um); mobile phase: 20% methanol (40 mM NH₃)/liquid CO₂, at a flow rate of 70 mL/min] resulting in Peak 1 and 2 fractions with enantiomeric excess >99%. Peak 2 correlates with the (S) enantiomer.
³Purified via preparative SFC [using a AD-H (2 × 15 cm); mobile phase: 20% methanol/liquid CO₂, at a flow rate of 60 mL/min] resulting in Peak 1 and 2 fractions with enantiomeric excess >99%. Peak 2 correlates with the (S) enantiomer.
⁴Purified via preparative SFC [using a AD-H (21 × 250 mm, 5 um); mobile phase: 12% methanol (40 mM NH₃)/liquid CO₂, at a flow rate of 70 mL/min] resulting in Peak 1 and 2 fractions with enantiomeric excess >99%. Peak 2 correlates with the (S) enantiomer.
⁵Purified via preparative SFC [using a Chiralcel OJH (21 × 250 mm, 5 um); mobile phase: 20% ethanol (20 mM NH₃)/liquid CO₂, at a flow rate of 75 mL/min] resulting in Peak 1 and 2 fractions with enantiomeric excess >98%. Peak 2 correlates with the (S) enantiomer.
⁶Purified via preparative SFC [using a AD-H column (250 × 20 mm); mobile phase: 20% methanol (20 mM NH₃)/liquid CO₂, at a flow rate of 75 mL/min] resulting in Peak 1 and 2 fractions with enantiomeric excess >98%. Peak 2 correlates with the (S) enantiomer.
⁷Purified via preparative SFC [using a AD-H column (250 × 20 mm); mobile phase: 20% methanol (20 mM NH₃)/liquid CO₂, at a flow rate of 75 mL/min] resulting in Peak 1 and 2 fractions with enantiomeric excess >98%. Peak 2 correlates with the (S) enantiomer.
⁸Purified via preparative SFC [using a AD-H column (250 × 20 mm); mobile phase: 20% methanol (20 mM NH₃)/liquid CO₂, at a flow rate of 75 mL/min] resulting in Peak 1 and 2 fractions with enantiomeric excess >98%. Peak 2 correlates with the (S) enantiomer.
⁹Purified via preparative SFC [using a AD-H column (250 × 20 mm); mobile phase: 10% methanol (20 mM NH₃)/liquid CO₂, at a flow rate of 75 mL/min] resulting in Peak 1 and 2 fractions with enantiomeric excess >98%. Peak 2 correlates with the (S) enantiomer.
¹⁰Purified via preparative SFC [using a AD-H column (250 × 20 mm); mobile phase: 20% methanol (20 mM NH₃)/liquid CO₂, at a flow rate of 75 mL/min] resulting in Peak 1 and 2 fractions with enantiomeric excess >98%. Peak 2 correlates with the (S) enantiomer.
¹¹Purified via preparative SFC [using a ODH column (21 × 250 mm, 5 um); mobile phase: 30% methanol (20 mM NH₃)/liquid CO₂, at a flow rate of 65 mL/min] resulting in Peak 1 and 2 fractions with enantiomeric excess >98%. Peak 2 correlates with the (S) enantiomer.
¹²Purified via preparative SFC [using a AD-H column (250 × 20 mm); mobile phase: 20% methanol/liquid CO₂, at a flow rate of 75 mL/min] resulting in Peak 1 and 2 fractions with enantiomeric excess >99%. Peak 2 correlates with the (S) enantiomer.
¹³Purified via preparative SFC [using a Chiralcel OJH (21 × 250 mm, 5 um); mobile phase: 20% ethanol (20 mM NH₃)/liquid CO₂, at a flow rate of 70 mL/min] resulting in Peak 1 and 2 fractions with enantiomeric excess >99%. Peak 2 correlates with the (S) enantiomer.

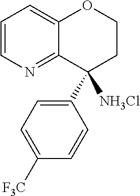

### Intermediate 15

### (S)-4-(4-(trifluoromethyl)phenyl)-3,4-dihydro-2H-pyrano[3,2-b]pyridin-4-amine

To a microwave vial with 4-methoxy-N-(4-(4-(trifluoromethyl)phenyl)-3,4-dihydro-2H-pyrano[3,2-b]pyridin-4-yl)benzamide (2 g, 4.67 mmol) was added HCl (5 M aqueous, 20 mL, 100 mmol). The vial was sealed and heated by microwave at 150° C. for 80 min. The reaction was then concentrated in vacuo and the residue dissolved in half saturated aqueous NaHCO₃ (75 mL) and DCM (50 mL). The organic layer was separated, and the aqueous layer was extracted with DCM (2×50 mL). The combined organic layers were washed with brine, dried over MgSO₄, and concentrated to give a green oil. Purification by flash chromatography (40 g SiO₂, 10-100% acetone in hexanes) gave racemic title compound. 4-(4-(trifluoromethyl)phenyl)-3,4-dihydro-2H-pyrano[3,2-b]pyridin-4-amine was separated by preparative SFC (using a AD-H (2×15 cm); mobile phase: 12% isopropanol (DEA)/CO₂, at a flow rate of 60 mL/min] resulting in Peak 1 and 2 fractions with enantiomeric excess >99%).

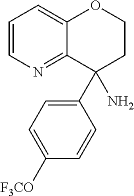 [SMILES: NC1(c2ccc(OC(F)(F)F)cc2)CCOc2cccnc21]

### Intermediate 16

### 4-(4-(trifluoromethoxy)phenyl)-3,4-dihydro-2H-pyrano[3,2-b]pyridin-4-amine

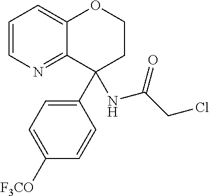 [SMILES: O=C(CCl)NC1(c2ccc(OC(F)(F)F)cc2)CCOc2cccnc21]

Step 1. 2-chloro-N-(4-(4-(trifluoromethoxy)phenyl)-3,4-dihydro-2H-pyrano[3,2-b]pyridin-4-yl)acetamide

To a stirred mixture of 4-(4-(trifluoromethoxy)phenyl)-3,4-dihydro-2H-pyrano[3,2-b]pyridin-4-ol (3 g. 9.6 mmol, Intermediate 3) and 2-chloroacetonitrile (7.2 mL, 128.5 mmol) at 0° C., were added acetic acid (8.68 mL) and conc. H₂SO₄ (9.45 mL). The ice bath was then removed, and the reaction was stirred at room temperature for 12 h. The progress of reaction was monitored by TLC (50% EtOAc in petroleum ether). The reaction was diluted with H₂O (20 mL) and EtOAc (40 mL) and quenched slowly with saturated NaHCO₃ (125 mL). The organic layer was separated and the aqueous layer extracted with EtOAc (2×50 mL). The combined organic layers were washed with brine (50 mL), dried over MgSO₄, and concentrated to give the product as a residue. Purification by column chromatography (60-120 mesh silica, 0-50% EtOAc in hexanes) gives the title compound as a white solid. MS (ESI pos. ion) m/z: 387.0 (MH+).

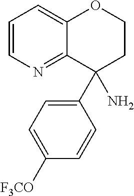 [SMILES: NC1(c2ccc(OC(F)(F)F)cc2)CCOc2cccnc21]

Step 2. 4-(4-(trifluoromethoxy)phenyl)-3,4-dihydro-2H-pyrano[3,2-b]pyridin-4-amine

To a stirred solution of 2-chloro-N-(4-(4-(trifluoromethoxy)phenyl)-3,4-dihydro-2H-pyrano[3,2-b]pyridin-4-yl)acetamide (1.5 g, 3.8 mmol) in EtOH (5 times) and AcOH (cat.), was added thiourea (0.3 g, 4.2 mmol). The reaction was stirred at 60° C. for 12 h. The progress of reaction was monitored by TLC (50% EtOAc in petroleum ether). The reaction was diluted with EtOAc (10 mL) and quenched slowly with saturated NaHCO₃ (25 mL). The aqueous layer was extracted with EtOAc (10 mL). The combined organic layers were washed with brine (10 mL), dried over MgSO₄, and concentrated to give the product as a residue. Purification by column chromatography (60-120 mesh silica, 0-50% EtOAc/hexanes) gives the title compound as a brown oil. MS (ESI pos. ion) m/z: 294.1 (M-NH₂). ¹H NMR (300 MHz, CDCl₃): δ 8.2 (d, J=4.0 Hz, 1H), 7.3 (m, 2H), 7.1-7.2 (m, 4H), 4.3 (m, 1H), 4.0 (m, 1H), 2.35-2.45 (m, 2H).

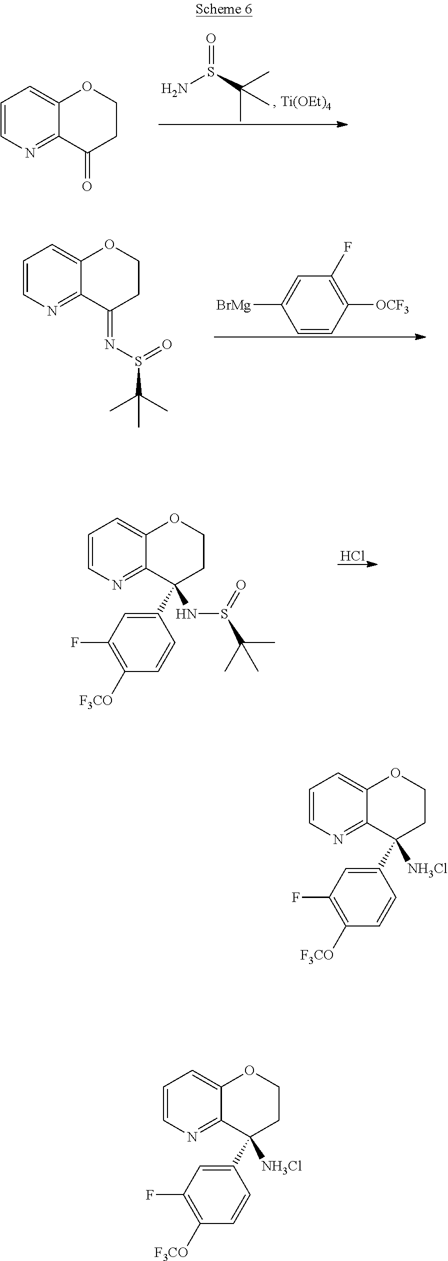

### Intermediate 17

### (S)-4-(3-fluoro-4-(trifluoromethoxy)phenyl)-3,4-dihydro-2H-pyrano[3,2-b]pyridin-4-amine hydrochloride

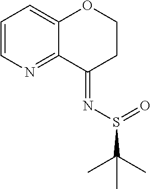 [SMILES: CC(C)(C)[S@](=O)/N=C1\CCOc2cccnc21]

Step 1. (S)-2-methyl-N-(2H-pyrano[3,2-b]pyridin-4(3H)-ylidene)propane-2-sulfinamide

To a solution of 2H-pyrano[3,2-b]pyridin-4(3H)-one (20.28 g, 136 mmol) and 2-MeTHF (200 mL) was added (S)-2-methylpropane-2-sulfinamide (24.72 g, 204 mmol) and titanium ethoxide (65 mL, 264 mmol). The solution was stirred at room temperature. After 4 h, the reaction was poured into rapidly stirring brine (500 mL). The suspension was stirred for 5 min and then EtOAc (200 mL) was poured into the stirring suspension. After stirring for a further 15 min, the suspension was filtered through a pad of Celite® brand filter agent. The solids were suspended again in water:EtOAc (1:1, 400 mL total). After stirring for 20 min, the suspension was filtered through a pad of Celite® brand filter agent. The solids were treated once more with EtOAc:water as previously described and filtered. The filtrate was separated, and the aqueous solution extracted with EtOAc (100 mL). The combined organic layers were concentrated in vacuo and adsorbed onto a plug of silica gel and chromatographed through a Redi-Sep® pre-packed silica gel column (330 g), eluting with 0-40% EtOAc in DCM, to provide the title compound as a golden oil. MS (ESI pos. ion) m/z: 253.1 (MH+).

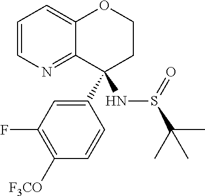 [SMILES: CC(C)(C)[S@](=O)N[C@]1(c2ccc(OC(F)(F)F)c(F)c2)CCOc2cccnc21]

Step 2. (S)—N—((S)-4-(3-fluoro-4-(trifluoromethoxy)phenyl)-3,4-dihydro-2H-pyrano[3,2-b]pyridin-4-yl)-2-methylpropane-2-sulfonamide

To an oven-dried round bottom flask was added magnesium (2.58 g, 106 mmol), iodine (˜25 mg), and 2-MeTHF (100 mL). The flask was placed in a water bath. The solution was treated with 10% 4-bromo-2-fluoro-1-(trifluoromethoxy)benzene (20.63 g, 80 mmol) and upon initiation was treated with the remaining bromide at a rate such that the temperature did not exceed 35° C. After stirring for 30 min. the Grignard solution was transferred dropwise to a solution of (S,E)-2-methyl-N-(2H-pyrano[3,2-b]pyridin-4(3H)-ylidene)propane-2-sulfinamide in 2-MeTHF (50 mL) that was cooled in a dry ice/acetone bath. The solution was stirred in the cooling bath and then the cooling bath was removed and the reaction was allowed to warm to room temperature. The reaction was quenched with MeOH (5 mL) and diluted with water (50 mL). After stirring for 30 minutes, the suspension was filtered through a pad of Celite® brand filter agent and the solids rinsed with EtOAc (3×20 mL). The filtrate was separated and the organic layers were concentrated in vacuo. The product thus obtained was adsorbed onto a plug of silica gel and chromatographed through a Redi-Sep® pre-packed silica gel column (80 g), eluting with 0-50% EtOAc in hexane, to provide the title compound as a golden oil. MS (ESI pos. ion) m/z: 433.0 (MH+).

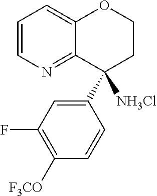

Step 3. (S)-4-(3-fluoro-4-(trifluoromethoxy)phenyl)-3,4-dihydro-2H-pyrano[3,2-b]pyridin-4-amine hydrochloride

To a solution of (S)—N—((S)-4-(3-fluoro-4-(trifluoromethoxy)phenyl)-3,4-dihydro-2H-pyrano[3,2-b]pyridin-4-yl)-2-methylpropane-2-sulfinamide (5.1 g, 11.79 mmol) and EtOH (30 mL) was added 4M HCl in dioxane (6 mL, 24.00 mmol). After 2 h, the reaction was concentrated in vacuo and triturated with ether (˜60 mL) and filtered. The solids were rinsed with more ether and dried in the funnel to give the title compound as an off-white solid. MS (ESI pos. ion) m/z: 329.0 (MH+). ¹H NMR (300 MHz, DMSO-d₆) δ: 9.33 (br. s., 3H), 8.35 (dd, J=3.8, 2.0 Hz, 1H), 7.59-7.75 (m, 2H), 7.42-7.54 (m, 2H), 7.00-7.13 (m, 1H), 4.34-4.51 (m, 1H), 3.77-3.85 (m, 1H), 2.70-2.88 (m, 1H), 2.65 (dd, J=11.8, 3.9 Hz, 1H).

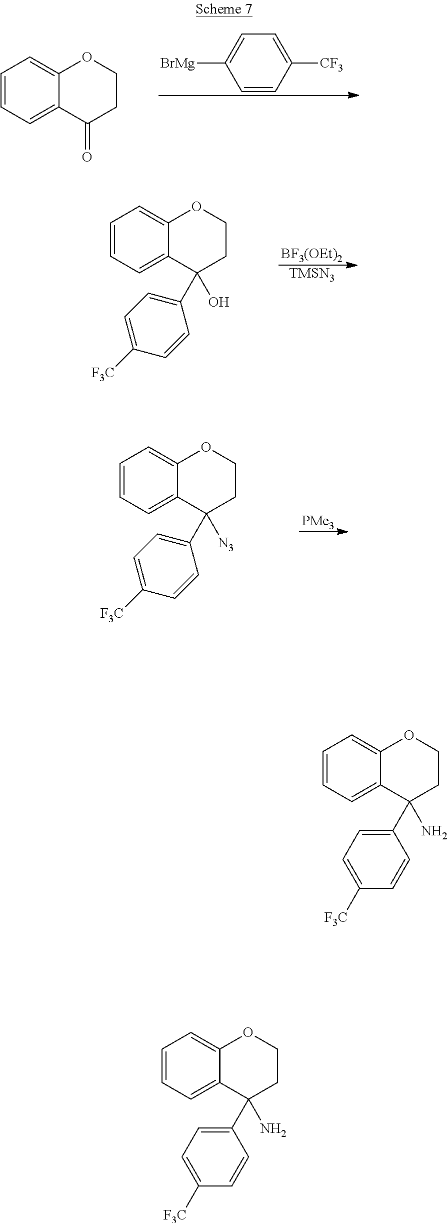

### Intermediate 18

### 4-(4-(trifluoromethyl)phenyl)chroman-4-amine

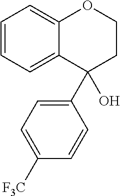 [SMILES: OC1(c2ccc(C(F)(F)F)cc2)CCOc2ccccc21]

Step 1. 4-(4-(trifluoromethyl)phenyl)chroman-4-ol

A round bottom flask with magnesium turnings (1.311 g, 54.0 mmol) and a crystal of I₂ was flame dried under N₂ until a purple gas was generated. The flask was cooled to room temperature and THF (30 mL) was added followed by 1-bromo-4-(trifluoromethyl)benzene (3.78 mL, 27.0 mmol). The reaction mixture turned dark brown after 15 min, and the resulting mixture was stirred under N₂ at room temperature for 2 h. The reaction mixture was cooled to 0° C. in a ice bath and then chroman-4-one (2.66 g, 17.98 mmol) in THF (20 mL) was added dropwise. The cooling bath was then removed and the solution allowed to warm to room temperature and stirred for 2.5 h. The reaction was quenched with saturated NH₄Cl (10 mL) and H₂O (10 mL). The mixture was then extracted with EtOAc (2×10 mL). The combined organic layers were dried (MgSO₄), and concentrated. Purification by flash chromatography (120 g SiO₂, 0-40% EtOAc/hexanes) gave the title compound as a yellow oil. MS (ESI pos. ion) m/z: 277.0 (M-OH). ¹H NMR (300 MHz, CDCl₃) δ: 7.47-7.65 (m, 4H), 7.23 (td, J=5.6, 2.6 Hz, 1H), 6.93 (d, J=8.2 Hz, 1H), 6.79-6.90 (m, 2H), 4.40 (td, J=11.1, 2.6 Hz, 1H), 4.27 (dt, J=11.3, 4.0 Hz, 1H), 2.25-2.38 (m, 2H), 2.12-2.23 (m, 1H).

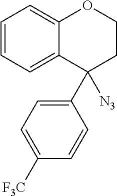 [SMILES: [N-]=[N+]=NC1(c2ccc(C(F)(F)F)cc2)CCOc2ccccc21]

Step 2. 4-azido-4-(4-(trifluoromethyl)phenyl)chroman

To a solution of 4-(4-(trifluoromethyl)phenyl)chroman-4-ol (112 mg, 0.381 mmol) in toluene (4 mL), was added an ice bath and TMS azide (0.101 mL, 0.761 mmol). After 5 min., the solution was treated with BF₃.OEt₂ (0.048 mL, 0.381 mmol) dropwise. The solution turned from clear to light yellow during the addition. After 20 min, the reaction was quenched with MeOH (2 mL) and diluted with EtOAc (20 mL). The organic solution was washed with water (2×10 mL) and the organic layer concentrated in vacuo to give the title compound as a light yellow oil. MS (ESI pos. ion) m/z: 277.0 (M-N₃). ¹H NMR (300 MHz, CDCl₃) δ: 7.57-7.65 (m, J=8.3 Hz, 2H), 7.41-7.52 (m, J=8.2 Hz, 2H), 7.26-7.33 (m, 1H), 6.81-7.03 (m, 3H), 4.30-4.44 (m, 1H), 4.21-4.30 (m, 1H), 2.23-2.34 (m, 1H), 2.12-2.23 (m, 1H).

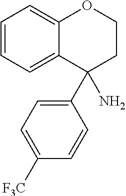 [SMILES: NC1(c2ccc(C(F)(F)F)cc2)CCOc2ccccc21]

Step 3. 4-(4-(trifluoromethyl)phenyl)chroman-4-amine

A solution of 4-azido-4-(4-(trifluoromethyl)phenyl)chroman (110 mg, 0.345 mmol) in THF (4 mL) was cooled in an ice bath. After 5 min., the solution was treated with 1 M trimethylphosphine (0.362 mL, 0.362 mmol) dropwise. After stirring for 20 min., the solution was diluted with MeOH (2 mL). The mixture was washed with water (5 mL). The organic layer was concentrated in vacuo to give the title compound in an unpurified form. MS (ESI pos. ion) m/z: 277.0 (M-NH₂).

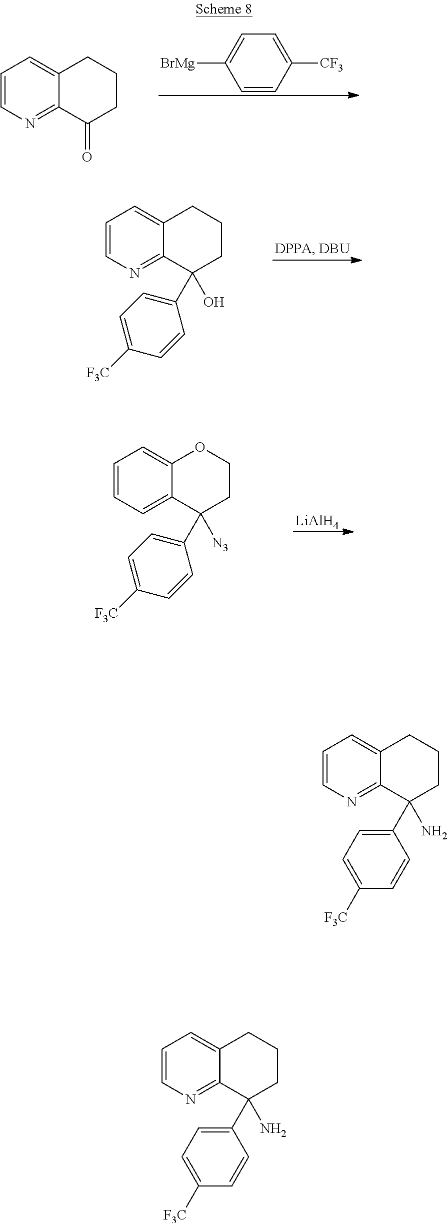

### Intermediate 19

### 8-(4-(trifluoromethyl)phenyl)-5,6,7,8-tetrahydroquinolin-8-amine

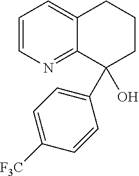 [SMILES: OC1(c2ccc(C(F)(F)F)cc2)CCCc2cccnc21]

Step 1. 8-(4-(trifluoromethyl)phenyl)-5,6,7,8-tetrahydroquinolin-8-ol

To a solution of magnesium (515 mg, 21.19 mmol) in THF (50 mL) was added 1M diisobutylaluminum hydride in hexanes (0.14 mL, 0.140 mmol). The solution was stirred at room temperature. After 20 min., the reaction was treated with a 20% portion of 1-bromo-4-(trifluoromethyl)benzene (1.9 mL, 13.57 mmol). After stirring for 20 min., the solution went from clear to light brown. The remaining 1-bromo-4-(trifluoromethyl)benzene was added dropwise. The solution was stirred for 0.5 h and then cooled in a dry ice/acetone bath. After cooling for 1 h, the reaction was treated with a solution of 6,7-dihydroquinolin-8(5H)-one (1.0 g, 6.79 mmol) in THF (7 mL) dropwise over 10 min. The solution was allowed to warm to room temperature as the cooling bath expires. After stirring for 16 h, the reaction was quenched with saturated aqueous NH₄Cl. The reaction was poured into water and EtOAc. The aqueous layer was extracted with EtOAc (25 mL). The combined EtOAc layers were concentrated in vacuo and adsorbed onto a plug of silica gel and chromatographed through a Redi-Sep® pre-packed silica gel column (40 g), eluting with 0-20% EtOAc in hexane, to provide the title compound as a colorless oil. MS (ESI pos. ion) m/z: 293.9 (MH+). ¹H NMR (300 MHz, CDCl₃) δ: 8.36-8.51 (m, 1H), 7.44-7.60 (m, 3H), 7.27 (s, 1H), 7.15-7.26 (m, 2H), 4.19 (s, 1H), 2.82-3.01 (m, 2H), 2.14-2.36 (m, 2H), 1.90 (dt, J=14.2, 4.5 Hz, 1H), 1.57-1.78 (m, 1H).

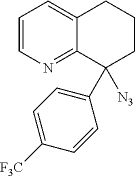 [SMILES: [N-]=[N+]=NC1(c2ccc(C(F)(F)F)cc2)CCCc2cccnc21]

Step 2. 8-azido-8-(4-(trifluoromethyl)phenyl)-5,6,7,8-tetrahydroquinoline

To a solution of 8-(4-(trifluoromethyl)phenyl)-5,6,7,8-tetrahydroquinolin-8-ol (1.2 g, 4.09 mmol) and toluene (2.5 mL) was added DPPA (2.7 mL, 12.53 mmol) and DBU (0.77 mL, 5.11 mmol). The reaction was then treated with sodium azide (780 mg, 12.00 mmol) and stirred for 2 days. The reaction was poured into a separatory funnel containing water and EtOAc. The aqueous layer was extracted with EtOAc (2×25 mL). The combined EtOAc layers were concentrated in vacuo and adsorbed onto a plug of silica gel and chromatographed through a Redi-Sep® pre-packed silica gel column (40 g), eluting with 0-40% EtOAc in hexane, to provide the title compound as a colorless oil. MS (ESI pos. ion) m/z: 319.0 (MH+).

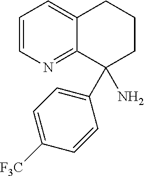 [SMILES: NC1(c2ccc(C(F)(F)F)cc2)CCCc2cccnc21]

Step 3. 8-(4-(trifluoromethyl)phenyl)-5,6,7,8-tetrahydroquinolin-8-amine

To a 0° C. solution of 8-azido-8-(4-(trifluoromethyl)phenyl)-5,6,7,8-tetrahydroquinoline (90 mg, 0.283 mmol) and THF (1 mL) was added 1M LiAlH₄ in THF (0.3 mL, 0.300 mmol) dropwise. After 30 min., the reaction was quenched with saturated aqueous potassium sodium tartrate salt. The reaction was diluted with EtOAc and the aqueous layer extracted with EtOAc (5 mL). The combined EtOAc layers were concentrated in vacuo to give the title compound as a yellow solid. MS (ESI pos. ion) m/z: 293.0 (MH+).

### Example 28

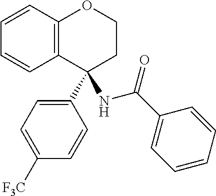 [SMILES: O=C(N[C@]1(c2ccc(C(F)(F)F)cc2)CCOc2ccccc21)c1ccccc1]

### (S)—N-(4-(4-(trifluoromethyl)phenyl)chroman-4-yl)benzamide

To a solution of 4-(4-(trifluoromethyl)phenyl)chroman-4-amine (0.345 mmol)(in an unpurified form) and DCM (8 mL) were added DIPEA (0.060 mL, 0.345 mmol) and benzoyl chloride (0.040 mL, 0.345 mmol). After stirring for 2 h, LC-MS showed ˜40% conversion. The reaction was treated with more benzoyl chloride (0.04 mL). After a further 1 h, the reaction was washed with water (5 mL). The organic layer was concentrated in vacuo, taken up in DCM (1.5 mL) and adsorbed onto a plug of silica gel and chromatographed through a Redi-Sep® pre-packed silica gel column (12 g), eluting with 0-10% EtOAc in hexane, to provide racemic compound as a white solid. The product was separated by preparative SFC (using a ASH (21×250 mm, Sum); mobile phase: 15% isopropanol (20 mM NH₃)/liquid CO₂, at a flow rate of 70 mL/min] resulting in Peak 1 and 2 fractions with enantiomeric excess >99%). Peak 2 correlates with the (S) enantiomer. MS (ESI, positive ion) m/z: 419.9 (MH+). ¹H NMR (300 MHz, CDCl₃) δ: 7.74-7.85 (m, 2H), 7.60 (d, J=8.3 Hz, 2H), 7.50-7.58 (m, 1H), 7.41-7.50 (m, 4H), 7.18-7.24 (m, 1H), 6.93 (dd, J=8.3, 0.9 Hz, 1H), 6.71-6.88 (m, 2H), 6.50 (s, 1H), 4.23-4.47 (m, 2H), 3.54-3.70 (m, 1H), 2.58 (ddd, J=14.5, 10.5, 4.2 Hz, 1H).

### Example 29

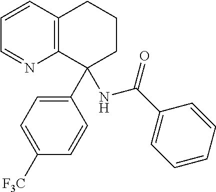 [SMILES: O=C(NC1(c2ccc(C(F)(F)F)cc2)CCCc2cccnc21)c1ccccc1]

### N-(8-(4-(trifluoromethyl)phenyl)-5,6,7,8-tetrahydroquinolin-8-yl)benzamide

To a 0° C. solution of 8-(4-(trifluoromethyl)phenyl)-5,6,7,8-tetrahydroquinolin-8-amine (62 mg, 0.136 mmol) and DCM (1 mL) was added DIPEA (0.035 mL, 0.201 mmol) and then benzoyl chloride (0.0175 mL, 0.151 mmol) dropwise. After 1 h, the reaction was poured into water and the aqueous layer back extracted with DCM (5 mL). The combined DCM layers were concentrated in vacuo and adsorbed onto a plug of silica gel and chromatographed through a Redi-Sep® pre-packed silica gel column (12 g), eluting with 0-30% EtOAc in hexane, to provide the title compound as a white solid. MS (ESI, positive ion) m/z: 397.0 (MH+). ¹H NMR (300 MHz, CDCl₃) δ: 8.46-8.63 (m, 2H), 7.74-7.88 (m, 2H), 7.33-7.66 (m, 8H), 7.24-7.28 (m, 1H), 3.55 (dt, J=14.0, 4.2 Hz, 1H), 2.89 (dd, J=9.6, 6.8 Hz, 1H), 2.54-2.82 (m, 2H), 1.86-2.01 (m, 1H), 1.65-1.83 (m, 1H).

### Example 59

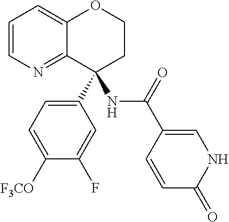 [SMILES: O=C(N[C@]1(c2ccc(OC(F)(F)F)c(F)c2)CCOc2cccnc21)c1ccc(=O)[nH]c1]

### (S)—N-(4-(3-fluoro-4-(trifluoromethoxy)phenyl)-3,4-dihydro-2H-pyrano[3,2-b]pyridin-4-yl)-6-oxo-1,6-dihydropyridine-3-carboxamide

A mixture of (S)-4-(3-fluoro-4-(trifluoromethoxy)phenyl)-3,4-dihydro-2H-pyrano[3,2-b]pyridin-4-amine hydrochloride (250 mg, 0.685 mmol), 6-hydroxypyridine-3-carboxylic acid (119 mg, 0.857 mmol), HOBt (116 mg, 0.857 mmol) and EDCI (164 mg, 0.857 mmol) in DMF (4 mL) was treated with TEA (0.100 mL, 0.720 mmol). The reaction was stirred at room temperature. After 6 h, the solution was diluted with EtOAc (75 mL) and washed with water (50 mL) and brine (50 mL), dried over MgSO₄, and concentrated in vacuo and purified by silica gel chromatography (eluent: 1-6% MeOH in DCM), affording the title compound as a white solid. MS (ESI, positive ion) m/z: 449.9 (MH+). ¹H NMR (400 MHz, DMSO-d₆) δ: 11.93 (br. s., 1H), 8.63 (s, 1H), 7.98-8.24 (m, 2H), 7.78 (dd, J=2.74, 9.8 Hz, 1H), 7.41-7.58 (m, 2H), 7.20-7.34 (m, 2H), 7.08-7.20 (m, 1H), 6.30 (d, J=9.8 Hz, 1H), 4.21-4.39 (m, 1H), 3.99-4.14 (m, 1H), 3.25 (dd, J=2.7, 7.4 Hz, 1H), 2.69-2.80 (m, 1H).

### General Amide Formation Procedure for Examples 30-63

To a solution of an amine or amine hydrochloride, the corresponding carboxylic acid (1.2 eq), and DIPEA (2 eq) in DCM or DMF (1 mL) at room temperature, was added an amide coupling reagent such as (HATU, TBTU, T3P, or EDCI) (1.2 eq.). The reaction was stirred for 1-16 h at room temperature. The reaction was then purified by one of the following methods: Method A: The reaction was diluted with DMF (1 mL), filtered through a syringe filter, and then purified by preparative reverse phase HPLC (gradient elution 10-100% MeCN/0.1% TFA in H₂O). The product-containing fractions were then combined and the solvent was removed by lyophilization to provide the target compound as the TFA salt; Method B: The reaction was diluted with DMF (1 mL), filtered through a syringe filter, and then purified by preparative reverse phase HPLC (gradient elution 10-100% MeCN/0.1% TFA in H₂O). The product-containing fractions were combined and concentrated, and the resulting product was dissolved in MeOH (1 mL) and washed through StratoSpheres® PL-HCO₃ MP-resin, and the resin was further washed with MeOH (2×0.4 mL). The combined filtrates were then concentrated and dried in vacuo to give the title compounds as free bases; Method C: After purification by reverse phase HPLC the product-containing fractions were concentrated, the solids dissolved in DCM, and the organic layer was extracted with saturated aqueous NaHCO₃, dried, and concentrated to provide the title compounds as free bases. Method D: The reaction mixture was diluted with H₂O and extracted with EtOAc (3×). The combined organic layers were dried (MgSO₄) and concentrated. Purification by silica flash chromatography provided the title compounds as free bases. Method E: The reactions were filtered and purified by mass directed preparative reverse phase HPLC (gradient elution 10-100% MeCN/0.1% formic acid in H₂O/0.1% formic acid). The product-containing fractions were combined and concentrated to provide the title compounds as formic acid salts.

TABLE 2
Examples 30-63 were prepared via amide formation analogous to the procedures described above.

| Ex | | | | | MS |
|---|---|---|---|---|---|
| # | Amine Intermediate | Acid Structure | Product Structure | Product Name | M + H |
| 30 | 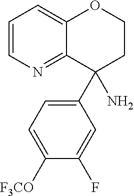 [SMILES: NC1(c2ccc(OC(F)(F)F)c(F)c2)CCOc2cccnc21] | 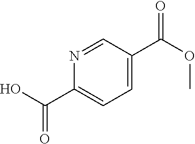 | 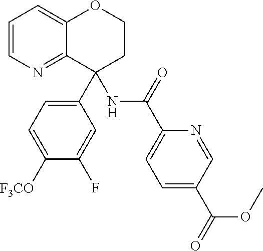 [SMILES: COC(=O)c1ccc(C(=O)NC2(c3ccc(OC(F)(F)F)c(F)c3)CCOc3cccnc32)nc1] | methyl 6-((4-(3- fluoro-4-(trifluoro- methoxy)phenyl)- 3,4-dihydro-2H- pyrano[3,2-b]pyridin- 4-yl)carbamoyl)nico- tinate | 492.0 |
| 31 | 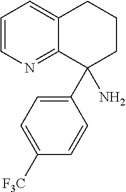 [SMILES: NC1(c2ccc(C(F)(F)F)cc2)CCCc2cccnc21] | 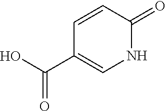 | 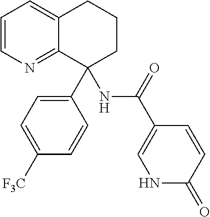 [SMILES: O=C(NC1(c2ccc(C(F)(F)F)cc2)CCCc2cccnc21)c1ccc(=O)[nH]c1] | 6-oxo-N-(8-(4- (trifluorometh- yl)phenyl)-5,6,7,8- tetrahydroquinolin- 8-yl)-1,6- dihydropyridine- 3-carboxamide | 414.0 |
| 32 | 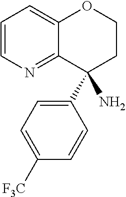 [SMILES: N[C@]1(c2ccc(C(F)(F)F)cc2)CCOc2cccnc21] | 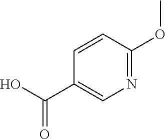 | 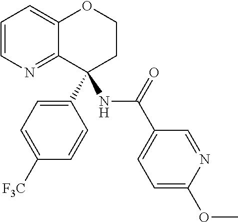 [SMILES: COc1ccc(C(=O)N[C@]2(c3ccc(C(F)(F)F)cc3)CCOc3cccnc32)cn1] | (S)-6-methoxy-N- (4-(4-(trifluoro- methyl)phenyl)-3,4- dihydro-2H- pyrano[3,2-b]pyridin- 4-yl)nicotinamide | 430.1 |
| 33 | 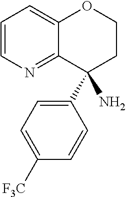 [SMILES: N[C@]1(c2ccc(C(F)(F)F)cc2)CCOc2cccnc21] | 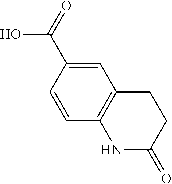 [SMILES: O=C1CCc2cc(C(=O)O)ccc2N1] | 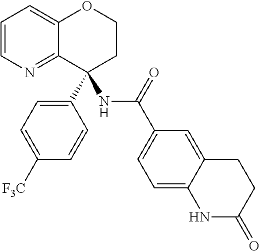 [SMILES: O=C1CCc2cc(C(=O)N[C@]3(c4ccc(C(F)(F)F)cc4)CCOc4cccnc43)ccc2N1] | (S)-2-oxo-N-(4-(4- (trifluorometh- yl)phenyl)-3,4- dihydro-2H- pyrano[3,2-b]pyridin- 4-yl)-1,2,3,4- tetrahydroquinoline- 6-carboxamide | 468.0 |
| 34 | 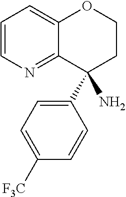 [SMILES: N[C@]1(c2ccc(C(F)(F)F)cc2)CCOc2cccnc21] | 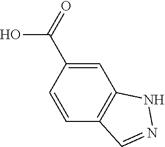 [SMILES: O=C(O)c1ccc2cn[nH]c2c1] | 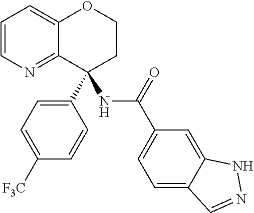 [SMILES: O=C(N[C@]1(c2ccc(C(F)(F)F)cc2)CCOc2cccnc21)c1ccc2cn[nH]c2c1] | (S)-N-(4-(4- (trifluorometh- yl)phenyl)-3,4- dihydro-2H- pyrano[3,2-b]pyridin- 4-yl)-1H-indazole-6- carboxamide | 439.1 |
| 35 | 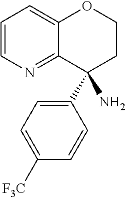 [SMILES: N[C@]1(c2ccc(C(F)(F)F)cc2)CCOc2cccnc21] | 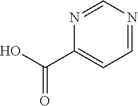 | 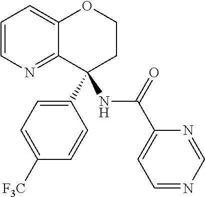 [SMILES: O=C(N[C@]1(c2ccc(C(F)(F)F)cc2)CCOc2cccnc21)c1ccncn1] | (S)-N-(4-(4- (trifluorometh- yl)phenyl)-3,4- dihydro-2H- pyrano[3,2-b]pyridin- 4-yl)pyrimidine-4- carboxamide | 401.0 |
| 36 | 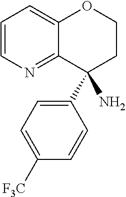 [SMILES: N[C@]1(c2ccc(C(F)(F)F)cc2)CCOc2cccnc21] | 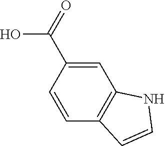 [SMILES: O=C(O)c1ccc2cc[nH]c2c1] | 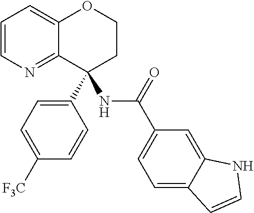 [SMILES: O=C(N[C@]1(c2ccc(C(F)(F)F)cc2)CCOc2cccnc21)c1ccc2cc[nH]c2c1] | (S)-N-(4-(4- (trifluorometh- yl)phenyl)-3,4- dihydro-2H- pyrano[3,2-b]pyridin- 4-yl)-1H-indole-6- carboxamide | 438.0 |
| 37 | 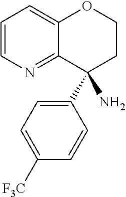 [SMILES: N[C@]1(c2ccc(C(F)(F)F)cc2)CCOc2cccnc21] | 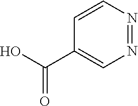 | 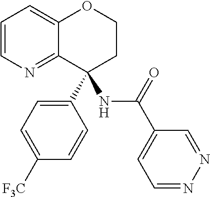 [SMILES: O=C(N[C@]1(c2ccc(C(F)(F)F)cc2)CCOc2cccnc21)c1ccnnc1] | (S)-N-(4-(4- (trifluorometh- yl)phenyl)-3,4- dihydro-2H- pyrano[3,2-b]pyridin- 4-yl)pyridazine-4- carboxamide | 401.1 |
| 38 | 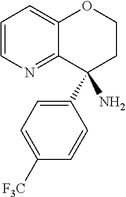 [SMILES: N[C@]1(c2ccc(C(F)(F)F)cc2)CCOc2cccnc21] | 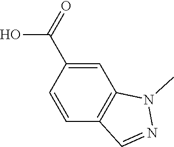 | 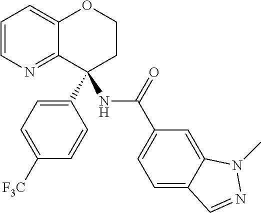 [SMILES: Cn1ncc2ccc(C(=O)N[C@]3(c4ccc(C(F)(F)F)cc4)CCOc4cccnc43)cc21] | (S)-1-methyl-N- (4-(4- (trifluorometh- yl)phenyl)-3,4- dihydro-2H- pyrano[3,2-b]pyridin- 4-yl)-1H-indazole-6- carboxamide | 453.0 |
| 39 | 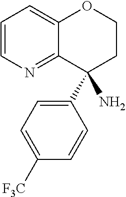 [SMILES: N[C@]1(c2ccc(C(F)(F)F)cc2)CCOc2cccnc21] | 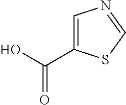 | 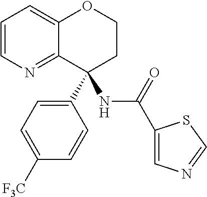 [SMILES: O=C(N[C@]1(c2ccc(C(F)(F)F)cc2)CCOc2cccnc21)c1cncs1] | (S)-N-(4-(4- (trifluorometh- yl)phenyl)-3,4- dihydro-2H- pyrano[3,2-b]pyridin- 4-yl)thiazole-5- carboxamide | 406.0 |
| 40 | 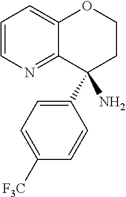 [SMILES: N[C@]1(c2ccc(C(F)(F)F)cc2)CCOc2cccnc21] | 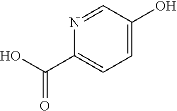 | 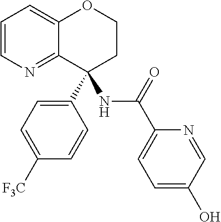 [SMILES: O=C(N[C@]1(c2ccc(C(F)(F)F)cc2)CCOc2cccnc21)c1ccc(O)cn1] | (S)-5-hydroxy-N-(4- (4-(trifluorometh- yl)phenyl)-3,4- dihydro-2H- pyrano[3,2-b]pyridin- 4-yl)picolinamide | 416.0 |
| 41 | 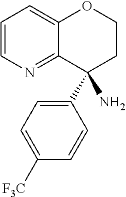 [SMILES: N[C@]1(c2ccc(C(F)(F)F)cc2)CCOc2cccnc21] | 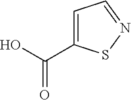 | 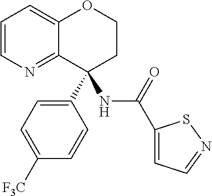 [SMILES: O=C(N[C@]1(c2ccc(C(F)(F)F)cc2)CCOc2cccnc21)c1ccns1] | (S)-N-(4-(4- (trifluorometh- yl)phenyl)-3,4- dihydro-2H- pyrano[3,2-b]pyridin- 4-yl)isothiazole-5- carboxamide | 406.0 |
| 42 | 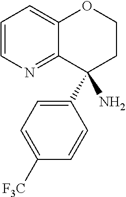 [SMILES: N[C@]1(c2ccc(C(F)(F)F)cc2)CCOc2cccnc21] | 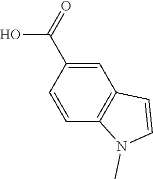 | 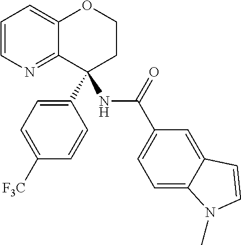 [SMILES: Cn1ccc2cc(C(=O)N[C@]3(c4ccc(C(F)(F)F)cc4)CCOc4cccnc43)ccc21] | (S)-1-methyl-N-(4- (4-(trifluorometh- yl)phenyl)-3,4- dihydro-2H- pyrano[3,2-b]pyridin- 4-yl)-1H-indole-5- carboxamide | 452.1 |
| 43 | 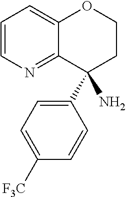 [SMILES: N[C@]1(c2ccc(C(F)(F)F)cc2)CCOc2cccnc21] | 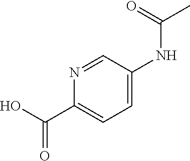 | 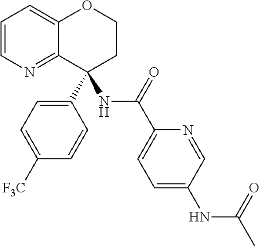 [SMILES: CC(=O)Nc1ccc(C(=O)N[C@]2(c3ccc(C(F)(F)F)cc3)CCOc3cccnc32)nc1] | (S)-5-acetamido-N- (4-(4-(trifluorometh- yl)phenyl)-3,4- dihydro-2H- pyrano[3,2-b]pyridin- 4-yl)picolinamide | 457.1 |
| 44 | 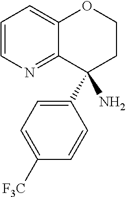 [SMILES: N[C@]1(c2ccc(C(F)(F)F)cc2)CCOc2cccnc21] | 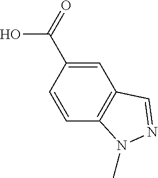 | 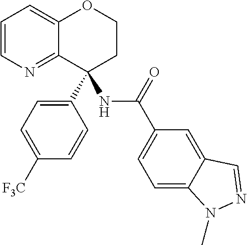 [SMILES: Cn1ncc2cc(C(=O)N[C@]3(c4ccc(C(F)(F)F)cc4)CCOc4cccnc43)ccc21] | (S)-1-methyl-N-(4- (4-(trifluorometh- yl)phenyl)-3,4- dihydro-2H- pyrano[3,2-b]pyridin- 4-yl)-1H-indazole-5- carboxamide | 453.0 |
| 45 | 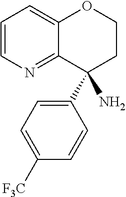 [SMILES: N[C@]1(c2ccc(C(F)(F)F)cc2)CCOc2cccnc21] | 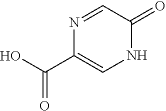 | 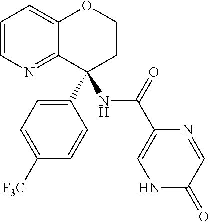 [SMILES: O=C(N[C@]1(c2ccc(C(F)(F)F)cc2)CCOc2cccnc21)c1c[nH]c(=O)cn1] | (S)-5-oxo-N-(4-(4- (trifluorometh- yl)phenyl)-3,4- dihydro-2H- pyrano[3,2-b]pyridin- 4-yl)-4,5- dihydropyrazine-2- carboxamide | 417.0 |
| 46 | 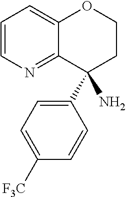 [SMILES: N[C@]1(c2ccc(C(F)(F)F)cc2)CCOc2cccnc21] | 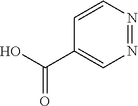 | 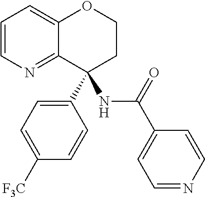 [SMILES: O=C(N[C@]1(c2ccc(C(F)(F)F)cc2)CCOc2cccnc21)c1ccncc1] | (S)-N-(4-(4- (trifluorometh- yl)phenyl)-3,4- dihydro-2H- pyrano[3,2-b]pyridin- 4-yl)isonicotinamide | 400.1 |
| 47 | 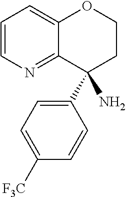 [SMILES: N[C@]1(c2ccc(C(F)(F)F)cc2)CCOc2cccnc21] | 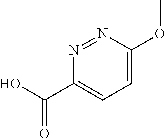 | 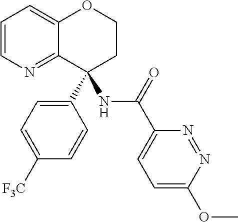 [SMILES: COc1ccc(C(=O)N[C@]2(c3ccc(C(F)(F)F)cc3)CCOc3cccnc32)nn1] | (S)-6-methoxy-N-(4- (4-(trifluorometh- yl)phenyl)-3,4- dihydro-2H- pyrano[3,2-b]pyridin- 4-yl)pyridazine-3- carboxamide | 431.1 |
| 48 | 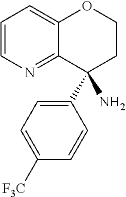 [SMILES: N[C@]1(c2ccc(C(F)(F)F)cc2)CCOc2cccnc21] | 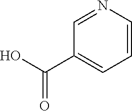 | 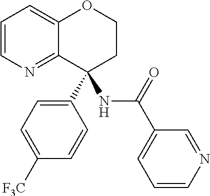 [SMILES: O=C(N[C@]1(c2ccc(C(F)(F)F)cc2)CCOc2cccnc21)c1cccnc1] | (S)-N-(4-(4- (trifluorometh- yl)phenyl)-3,4- dihydro-2H- pyrano[3,2-b]pyridin- 4-yl)nicotinamide | 400.1 |
| 49 | 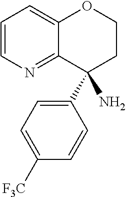 [SMILES: N[C@]1(c2ccc(C(F)(F)F)cc2)CCOc2cccnc21] | 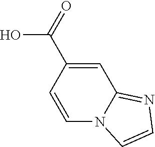 [SMILES: O=C(O)c1ccn2ccnc2c1] | 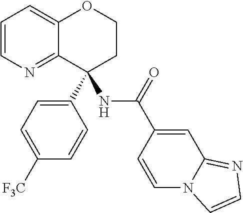 [SMILES: O=C(N[C@]1(c2ccc(C(F)(F)F)cc2)CCOc2cccnc21)c1ccn2ccnc2c1] | (S)-N-(4-(4- (trifluorometh- yl)phenyl)-3,4- dihydro-2H- pyrano[3,2-b]pyridin- 4- yl)imidazo[1,2-a]pyr- idine-7-carboxamide | 439.1 |
| 50 | 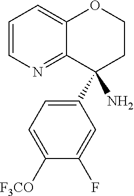 [SMILES: N[C@]1(c2ccc(OC(F)(F)F)c(F)c2)CCOc2cccnc21] | 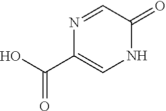 | 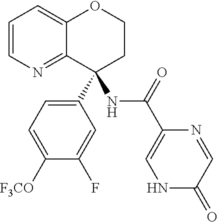 [SMILES: O=C(N[C@]1(c2ccc(OC(F)(F)F)c(F)c2)CCOc2cccnc21)c1c[nH]c(=O)cn1] | (S)-N-(4-(3-fluoro-4- (trifluoromethoxy) phenyl)-3,4-dihydro- 2H- pyrano[3,2-b]pyridin- 4-yl)-5-oxo-4,5- dihydropyrazine-2- carboxamide | 451.0 |
| 51 | 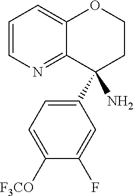 [SMILES: N[C@]1(c2ccc(OC(F)(F)F)c(F)c2)CCOc2cccnc21] | 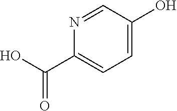 | 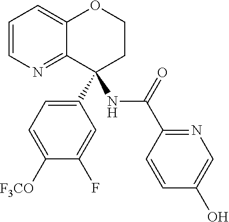 [SMILES: O=C(N[C@]1(c2ccc(OC(F)(F)F)c(F)c2)CCOc2cccnc21)c1ccc(O)cn1] | (S)-N-(4-(3-fluoro-4- (trifluoromethoxy) phenyl)-3,4-dihydro- 2H- pyrano[3,2-b]pyridin- 4-yl)-5- hydroxypicolinamide | 450.0 |
| 52 | 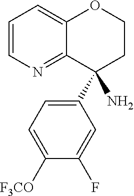 [SMILES: N[C@]1(c2ccc(OC(F)(F)F)c(F)c2)CCOc2cccnc21] | 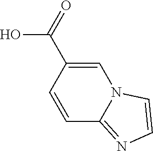 [SMILES: O=C(O)c1ccc2nccn2c1] | 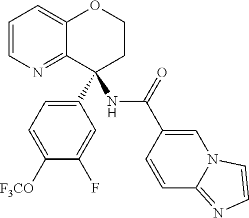 [SMILES: O=C(N[C@]1(c2ccc(OC(F)(F)F)c(F)c2)CCOc2cccnc21)c1ccc2nccn2c1] | (S)-N-(4-(3-fluoro-4- (trifluoromethoxy) phenyl)-3,4-dihydro- 2H- pyrano[3,2-b]pyridin- 4- yl)imidazo[1,2-a]pyr- idine-6- carboxamide | 473.0 |
| 53 | 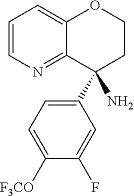 [SMILES: N[C@]1(c2ccc(OC(F)(F)F)c(F)c2)CCOc2cccnc21] | 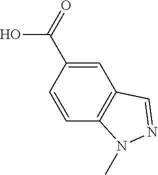 | 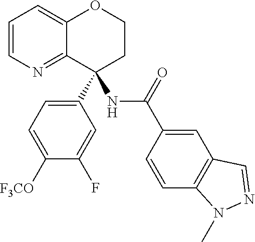 [SMILES: Cn1ncc2cc(C(=O)N[C@]3(c4ccc(OC(F)(F)F)c(F)c4)CCOc4cccnc43)ccc21] | (S)-N-(4-(3-fluoro-4- (trifluoromethoxy) phenyl)-3,4- dihydro-2H- pyrano[3,2-b]pyridin- 4-yl)-1-methyl-1H- indazole-5- carboxamide | 487.1 |
| 54 | 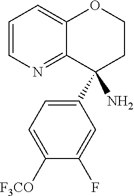 [SMILES: N[C@]1(c2ccc(OC(F)(F)F)c(F)c2)CCOc2cccnc21] | 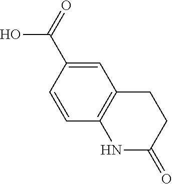 [SMILES: O=C1CCc2cc(C(=O)O)ccc2N1] | 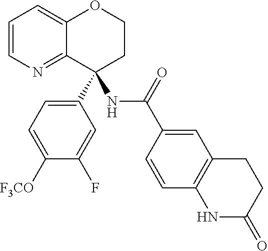 [SMILES: O=C1CCc2cc(C(=O)N[C@]3(c4ccc(OC(F)(F)F)c(F)c4)CCOc4cccnc43)ccc2N1] | (S)-N-(4-(3-fluoro-4- (trifluoromethoxy) phenyl)-3,4- dihydro-2H- pyrano[3,2-b]pyridin- 4-yl)-2-oxo-1,2,3,4- tetrahydroquinoline- 6-carboxamide | 502.0 |
| 55 | 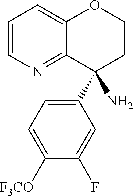 [SMILES: N[C@]1(c2ccc(OC(F)(F)F)c(F)c2)CCOc2cccnc21] | 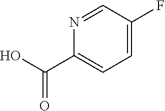 | 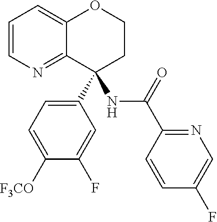 [SMILES: O=C(N[C@]1(c2ccc(OC(F)(F)F)c(F)c2)CCOc2cccnc21)c1ccc(F)cn1] | (S)-5-fluoro-N-(4-(3- fluoro-4-(trifluoro- methoxy)phenyl)- 3,4-dihydro-2H- pyrano[3,2-b]pyridin- 4-yl)picolinamide | 452.0 |
| 56 | 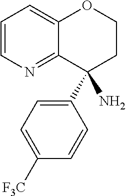 [SMILES: N[C@]1(c2ccc(C(F)(F)F)cc2)CCOc2cccnc21] | 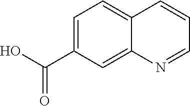 [SMILES: O=C(O)c1ccc2cccnc2c1] | 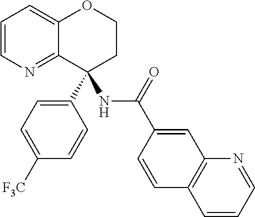 [SMILES: O=C(N[C@]1(c2ccc(C(F)(F)F)cc2)CCOc2cccnc21)c1ccc2cccnc2c1] | (S)-N-(4-(4- (trifluorometh- yl)phenyl)-3,4- dihydro-2H- pyrano[3,2-b]pyridin- 4-yl)quinoline-7- carboxamide | 449.8 |
| 57 | 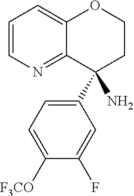 [SMILES: N[C@]1(c2ccc(OC(F)(F)F)c(F)c2)CCOc2cccnc21] | 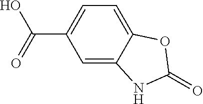 [SMILES: O=C(O)c1ccc2oc(=O)[nH]c2c1] | 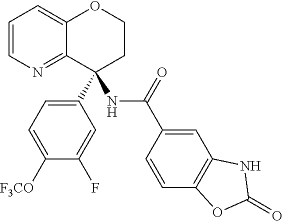 [SMILES: O=C(N[C@]1(c2ccc(OC(F)(F)F)c(F)c2)CCOc2cccnc21)c1ccc2oc(=O)[nH]c2c1] | (S)-N-(4-(3-fluoro-4- (trifluorometh- oxy)phenyl)-3,4- dihydro-2H- pyrano[3,2-b]pyridin- 4-yl)-2-oxo-2,3- dihydro- benzo[d]oxazole-5- carboxamide | 489.6 |
| 58¹ | 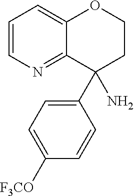 [SMILES: NC1(c2ccc(OC(F)(F)F)cc2)CCOc2cccnc21] | 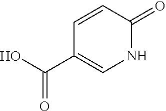 | 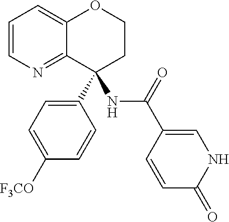 [SMILES: O=C(N[C@]1(c2ccc(OC(F)(F)F)cc2)CCOc2cccnc21)c1ccc(=O)[nH]c1] | (S)-6-oxo-N-(4-(4- (trifluorometh- oxy)phenyl)-3,4- dihydro-2H- pyrano[3,2-b]pyridin- 4-yl)-1,6- dihydropyridine-3- carboxamide | 431.7 |
| 59 | 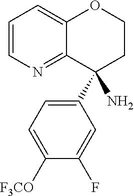 [SMILES: N[C@]1(c2ccc(OC(F)(F)F)c(F)c2)CCOc2cccnc21] | 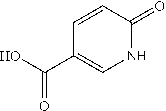 | 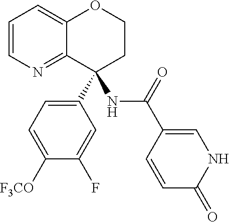 [SMILES: O=C(N[C@]1(c2ccc(OC(F)(F)F)c(F)c2)CCOc2cccnc21)c1ccc(=O)[nH]c1] | (S)-N-(4-(3-fluoro-4- (trifluoromethoxy) phenyl)-3,4- dihydro-2H- pyrano[3,2-b]pyridin- 4-yl)-6-oxo-1,6- dihydropyridine- 3-carboxamide | 449.8 |
| 60 | 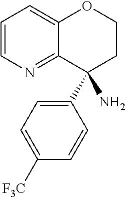 [SMILES: N[C@]1(c2ccc(C(F)(F)F)cc2)CCOc2cccnc21] | 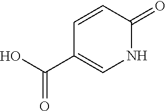 | 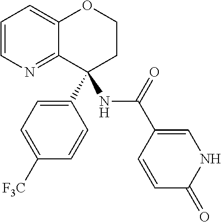 [SMILES: O=C(N[C@]1(c2ccc(C(F)(F)F)cc2)CCOc2cccnc21)c1ccc(=O)[nH]c1] | (S)-6-oxo-N-(4-(4- (trifluorometh- yl)phenyl)-3,4- dihydro-2H- pyrano[3,2-b]pyridin- 4-yl)-1,6- dihydropyridine-3- carboxamide | 416.1 |
| 61 | 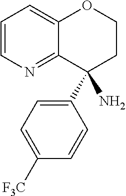 [SMILES: N[C@]1(c2ccc(C(F)(F)F)cc2)CCOc2cccnc21] | 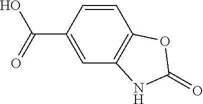 [SMILES: O=C(O)c1ccc2oc(=O)[nH]c2c1] | 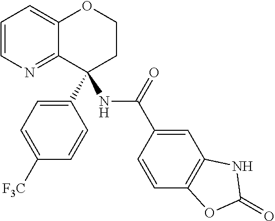 [SMILES: O=C(N[C@]1(c2ccc(C(F)(F)F)cc2)CCOc2cccnc21)c1ccc2oc(=O)[nH]c2c1] | (S)-2-oxo-N-(4-(4- (trifluorometh- yl)phenyl)-3,4- dihydro-2H- pyrano[3,2-b]pyridin- 4-yl)-2,3-dihydro- benzo[d]oxazole-5- carboxamide | 456.0 |
| 62 | 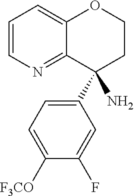 [SMILES: N[C@]1(c2ccc(OC(F)(F)F)c(F)c2)CCOc2cccnc21] | 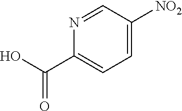 [SMILES: O=C(O)c1ccc([N+](=O)[O-])cn1] | 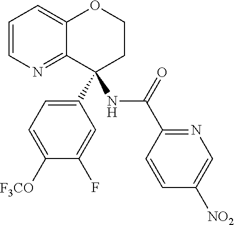 [SMILES: O=C(N[C@]1(c2ccc(OC(F)(F)F)c(F)c2)CCOc2cccnc21)c1ccc([N+](=O)[O-])cn1] | (S)-N-(4-(3-fluoro-4- (trifluoromethoxy) phenyl)-3,4- dihydro-2H- pyrano[3,2-b]pyridin- 4-yl)-5- nitropicolinamide | 479.0 |
| 63 | 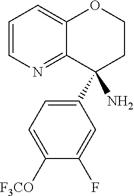 [SMILES: N[C@]1(c2ccc(OC(F)(F)F)c(F)c2)CCOc2cccnc21] | 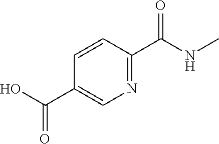 | 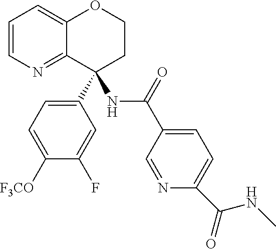 [SMILES: CNC(=O)c1ccc(C(=O)N[C@]2(c3ccc(OC(F)(F)F)c(F)c3)CCOc3cccnc32)cn1] | (S)-N5-(4-(3-fluoro- 4-(trifluorometh- oxy)phenyl)-3,4- dihydro-2H- pyrano[3,2-b]pyridin- 4-yl)-N2- methylpyridine-2,5- dicarboxamide | 491.0 |

¹Purified via preparative SFC [using a ChiralpakAD-H (250 × 30 mm, 5 um); mobile phase: 30% methanol/liquid CO₂, at a flow rate of 120 mL/min] resulting in Peak 1 and 2 fractions with enantiomeric excess >99%. Peak 2 correlates with the (S) enantiomer.

### Example 64

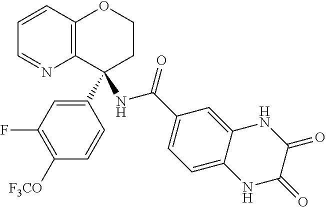 [SMILES: O=C(N[C@]1(c2ccc(OC(F)(F)F)c(F)c2)CCOc2cccnc21)c1ccc2[nH]c(=O)c(=O)[nH]c2c1]

### (S)—N-(4-(3-fluoro-4-(trifluoromethoxy)phenyl)-3,4-dihydro-2H-pyrano[3,2-b]pyridin-4-yl)-2,3-dioxo-1,2,3,4-tetrahydroquinoxaline-6-carboxamide

To a solution of 2,3-dioxo-1,2,3,4-tetrahydroquinoxaline-6-carboxylic acid (0.300 g, 1.455 mmol) in DCM (3 mL) was added oxalyl chloride (0.387 mL, 4.37 mmol) a drop of DMF (anhydrous). The resulting mixture was then stirred at room temperature for 2 h, then the mixture was concentrated in vacuo to give 2,3-dioxo-1,2,3,4-tetrahydroquinoxaline-6-carbonyl chloride. A solution of (S)-4-(3-fluoro-4-(trifluoromethoxy)phenyl)-3,4-dihydro-2H-pyrano[3,2-b]pyridin-4-amine hydrochloride (50 mg, 0.137 mmol) in DCM (1 mL) at 0° C. was added 2,3-dioxo-1,2,3,4-tetrahydroquinoxaline-6-carbonyl chloride (326 mg, 1.453 mmol), and DIPEA (0.072 mL, 0.411 mmol). The resulting mixture was stirred at 0° C. for 30 min and then at room temperature for overnight. The reaction was diluted with H₂O (5 mL) and EtOAc (7 mL). The mixture was then stirred at room temperature for 30 min. The mixture was then filtered and the organic layer was dried over MgSO₄, and concentrated in vacuo. The residue was dissolved in DMSO (2 mL) and solution was purified by preparative HPLC (0-100% MeCN 0.1% TFA/H₂O 0.1% TFA) to give the title compound as a white solid. MS (ESI, positive ion) m/z: 517.0 (MH+). ¹HNMR (400 MHz, d₄-MeOH) δ: 8.27 (dd, J=4.8, 1.3 Hz, 1H), 7.60-7.69 (m, 3H), 7.51-7.58 (m, 1H), 7.42-7.48 (m, 2H), 7.24 (d, J=8.4 Hz, 2H), 4.45-4.59 (m, 1H), 4.16-4.35 (m, 1H), 3.07-3.27 (m, 1H), 2.84-3.05 (m, 1H).

### Example 65

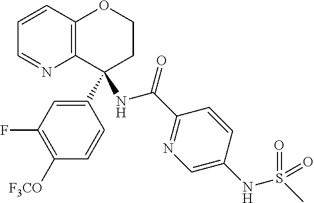 [SMILES: CS(=O)(=O)Nc1ccc(C(=O)N[C@]2(c3ccc(OC(F)(F)F)c(F)c3)CCOc3cccnc32)nc1]

### (S)—N-(4-(3-fluoro-4-(trifluoromethoxy)phenyl)-3,4-dihydro-2H-pyrano[3,2-b]pyridin-4-yl)-5-(methylsulfonamido)picolinamide

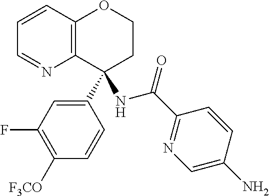 [SMILES: Nc1ccc(C(=O)N[C@]2(c3ccc(OC(F)(F)F)c(F)c3)CCOc3cccnc32)nc1]

Step 1: (S)-5-amino-N-(4-(3-fluoro-4-(trifluoromethoxy)phenyl)-3,4-dihydro-2H-pyrano[3,2-b]pyridin-4-yl)picolinamide

To a solution of (S)—N-(4-(3-fluoro-4-(trifluoromethoxy)phenyl)-3,4-dihydro-2H-pyrano[3,2-b]pyridin-4-yl)-5-nitropicolinamide (105 mg, 0.220 mmol, Example 62) in MeOH (1.3 mL) was added a solution of Pd on carbon (0.012 mL, 0.110 mmol) in EtOAc (0.3 mL). The resulting mixture was stirred at room temperature under H₂ (balloon) for 2 h. The mixture was filtered through Celite® brand filter agent and the Celite® brand filter agent was washed with MeOH (2×2 mL). The filtrate was concentrated in vacuo and the residue purified by silica gel column chromatography using flash chromatography instrument (70-100% EtOAc/hexane) to give the title compound as a yellow solid. MS (ESI, positive ion) m/z: 449.0 (MH+).

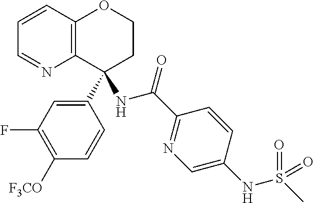 [SMILES: CS(=O)(=O)Nc1ccc(C(=O)N[C@]2(c3ccc(OC(F)(F)F)c(F)c3)CCOc3cccnc32)nc1]

Step 2. (S)—N-(4-(3-fluoro-4-(trifluoromethoxy)phenyl)-3,4-dihydro-2H-pyrano[3,2-b]pyridin-4-yl)-5-(methylsulfonamido)picolinamide

To a solution of (S)-5-amino-N-(4-(3-fluoro-4-(trifluoromethoxy)phenyl)-3,4-dihydro-2H-pyrano[3,2-b]pyridin-4-yl)picolinamide (44 mg, 0.098 mmol) in DCM (0.6 mL) were added methanesulfonyl chloride (8.40 μL, 0.128 mmol) and DIPEA (0.022 mL, 0.128 mmol). The resulting mixture was stirred at room temperature overnight. A solution of NaOH (10 N, 2 drops) was added, and the resulting mixture was stirred at room temperature for 2 h. The reaction mixture was filtered and the filtrate concentrated in vacuo. The residue was purified by prep HPLC (0-100% MeCN 0.1% TFA/H₂O 0.1% TFA) to give the title compound as a white solid. MS (ESI, positive ion) m/z: 527.0 (MH+). ¹H NMR (400 MHz, d₄-MeOH) δ: 8.48 (d, J=2.3 Hz, 1H), 8.30 (dd, J=4.5, 1.4 Hz, 1H), 8.05 (d, J=8.6 Hz, 1H), 7.83 (dd, J=8.6, 2.5 Hz, 1H), 7.37-7.62 (m, 4H), 7.25 (d, J=8.6 Hz, 1H), 4.47 (dt, J=11.9, 4.4 Hz, 1H), 4.05-4.26 (m, 1H), 3.12-3.25 (m, 2H), 3.10 (s, 3H).

### Example 66

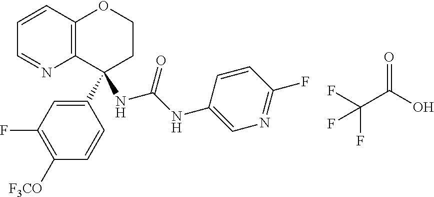

### (S)-1-(4-(3-fluoro-4-(trifluoromethoxy)phenyl)-3,4-dihydro-2H-pyrano[3,2-b]pyridin-4-yl)-3-(6-fluoropyridin-3-yl)urea 2,2,2-trifluoroacetate

To a solution of (S)-4-(3-fluoro-4-(trifluoromethoxy)phenyl)-3,4-dihydro-2H-pyrano[3,2-b]pyridin-4-amine hydrochloride (0.030 g, 0.082 mmol) in DCM (0.5 mL) were added 5-amino-2-fluoropyridine (0.014 g, 0.123 mmol), CDI (0.027 g, 0.165 mmol), and TEA (0.046 mL, 0.329 mmol). The resulting mixture was then stirred at room temperature for 18 h. The mixture was filtered and the filtrate was purified by preparative HPLC (0-100% MeCN 0.1% TFA/H2O 0.1% TFA) to give the title compound as a yellow solid. MS (ESI, positive ion) m/z: 467 (MH+). ¹H NMR (400 MHz, d₄-MeOH) δ: 8.29 (dd, J=4.8, 1.3 Hz, 1H), 8.15 (s, 1H), 7.97 (ddd, J=9.1, 6.7, 2.7 Hz, 1H), 7.60-7.67 (m, 1H), 7.52-7.58 (m, 1H), 7.35-7.49 (m, 2H), 7.20 (d, J=8.6 Hz, 1H), 6.99 (dd, J=9.0, 2.9 Hz, 1H), 4.48 (ddd, J=11.8, 6.2, 3.5 Hz, 1H), 4.18 (ddd, J=11.9, 9.5, 2.6 Hz, 1H), 3.04-3.20 (m, 1H), 2.78-2.90 (m, 1H).

### Example 67

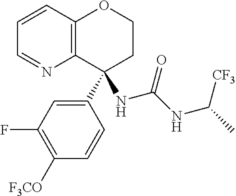 [SMILES: C[C@H](NC(=O)N[C@]1(c2ccc(OC(F)(F)F)c(F)c2)CCOc2cccnc21)C(F)(F)F]

### 1-((S)-4-(4-(trifluoromethyl)phenyl)-3,4-dihydro-2H-pyrano[3,2-b]pyridin-4-yl)-3-((S)-1,1,1-trifluoropropan-2-yl)urea 2,2,2-trifluoroacetate

To a solution of CDI (41.3 mg, 0.255 mmol) in DCM (0.25 mL) was added (S)-1,1,1-trifluoropropan-2-amine (0.026 mL, 0.255 mmol). The round bottom flask was then sealed, and the mixture was stirred at room temperature for 2.5 h. A solution of 4-(4-(trifluoromethyl)phenyl)-3,4-dihydro-2H-pyrano[3,2-b]pyridin-4-amine (50.0 mg, 0.170 mmol) and DIPEA (0.058 mL, 0.340 mmol) in DCM (1 mL) was then added, and the reaction was stirred at room temperature for 20 h. The reaction was diluted with DMF, filtered through a syringe filter, and the reaction purified by RPHPLC (Gemini Axia C18 50×150 mm column, gradient elution 10-100% MeCN/0.1% TFA in H₂O). The product containing fractions were combined and the solvent removed by lyophilization to give the title compound as a white solid. MS (ESI, positive ion) m/z: 433.9 (MH+). ¹H NMR (300 MHz, d₄-MeOH) δ: 8.18-8.29 (m, 1H), 7.64-7.71 (m, J=8.2 Hz, 2H), 7.59 (br. s., 1H), 7.47-7.56 (m, 1H), 7.34-7.47 (m, J=8.3 Hz, 2H), 4.25-4.46 (m, 2H), 4.06 (ddd, J=11.9, 9.6, 2.6 Hz, 1H), 3.02-3.20 (m, 1H), 2.54-2.72 (m, 1H), 1.19-1.30 (m, 3H).

### General Urea Formation Procedure for Examples 68-74

To a solution of chromane amine (1 eq.) in DCM was added the desired isocyanate (1.2 eq) and DIPEA (1 eq). The resulting mixture was then stirred at room temperature for 18 h. The mixture was then concentrated in vacuo and the product thus obtained was dissolved in DMSO (1 mL). The solution mixture was then purified by preparative HPLC (0%-100% MeCN 0.1% TFA/H2O 0.1% TFA) to give the title compound.

TABLE 3
Examples 68-74 were prepared via urea formation analogous to the procedures described above.

| | | | | | MS |
|---|---|---|---|---|---|
| Ex # | Amine Intermediate | Isocyanate Structure | Product Structure | Product Name | M + H |
| 68 | 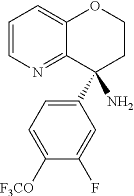 [SMILES: N[C@]1(c2ccc(OC(F)(F)F)c(F)c2)CCOc2cccnc21] | 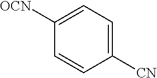 | 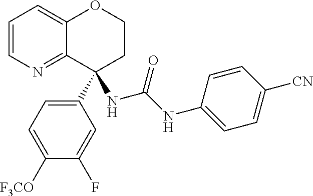 [SMILES: N#Cc1ccc(NC(=O)N[C@]2(c3ccc(OC(F)(F)F)c(F)c3)CCOc3cccnc32)cc1] | (S)-1-(4-cyanophenyl)- 3-(4-(3-fluoro-4- (trifluoromethoxy)phen- yl)-3,4-dihydro-2H- pyrano[3,2-b]pyridin-4- yl)urea | 473.0 |
| 69 | 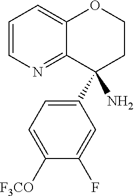 [SMILES: N[C@]1(c2ccc(OC(F)(F)F)c(F)c2)CCOc2cccnc21] | 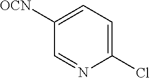 | 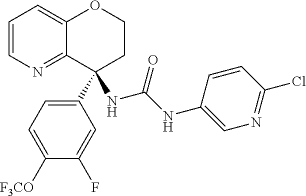 [SMILES: O=C(Nc1ccc(Cl)nc1)N[C@]1(c2ccc(OC(F)(F)F)c(F)c2)CCOc2cccnc21] | (S)-1-(6-chloropyridin- 3-yl)-3-(4-(3-fluoro-4- (trifluoromethoxy)phen- yl)-3,4-dihydro-2H- pyrano[3,2-b]pyridin-4- yl)urea | 483.0 |
| 70 | 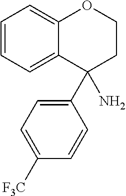 [SMILES: NC1(c2ccc(C(F)(F)F)cc2)CCOc2ccccc21] | 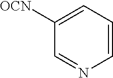 | 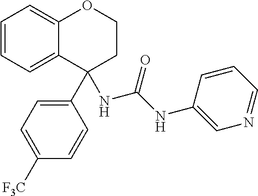 [SMILES: O=C(Nc1cccnc1)NC1(c2ccc(C(F)(F)F)cc2)CCOc2ccccc21] | 1-(pyridin-3-yl)-3-(4-(4- (trifluoromethyl)phen- yl)chroman-4-yl)urea | 414.0 |
| 71 | 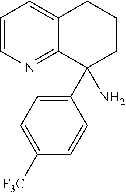 [SMILES: NC1(c2ccc(C(F)(F)F)cc2)CCCc2cccnc21] | 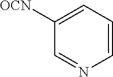 | 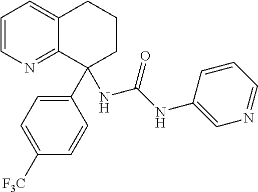 [SMILES: O=C(Nc1cccnc1)NC1(c2ccc(C(F)(F)F)cc2)CCCc2cccnc21] | 1-(pyridin-3-yl)-3-(8-(4- (trifluoromethyl)phenyl)- 5,6,7,8- tetrahydroquinolin-8- yl)urea | 413.0 |
| 72 | 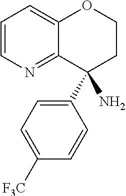 [SMILES: N[C@]1(c2ccc(C(F)(F)F)cc2)CCOc2cccnc21] | 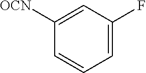 | 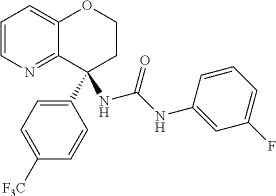 [SMILES: O=C(Nc1cccc(F)c1)N[C@]1(c2ccc(C(F)(F)F)cc2)CCOc2cccnc21] | (S)-1-(3-fluorophenyl)-3- (4-(4- (trifluoromethyl)phenyl)- 3,4-dihydro-2H- pyrano[3,2-b]pyridin-4- yl)urea | 431.7 |
| 73 | 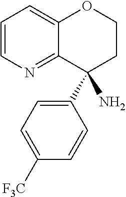 [SMILES: N[C@]1(c2ccc(C(F)(F)F)cc2)CCOc2cccnc21] | 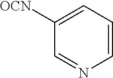 | 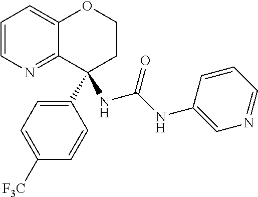 [SMILES: O=C(Nc1cccnc1)N[C@]1(c2ccc(C(F)(F)F)cc2)CCOc2cccnc21] | (S)-1-(pyridin-3-yl)-3-(4- (4-(trifluoromethyl)phen- yl)-3,4-dihydro-2H- pyrano[3,2-b]pyridin-4- yl)urea | 414.8 |
| 74 | 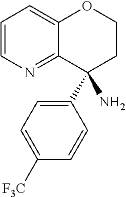 [SMILES: N[C@]1(c2ccc(C(F)(F)F)cc2)CCOc2cccnc21] | 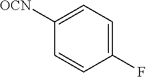 | 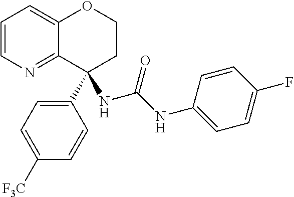 [SMILES: O=C(Nc1ccc(F)cc1)N[C@]1(c2ccc(C(F)(F)F)cc2)CCOc2cccnc21] | (S)-1-(4-fluorophenyl)-3- (4-(4- (trifluoromethyl)phenyl)- 3,4-dihydro-2H- pyrano[3,2-b]pyridin-4- yl)urea | 431.9 |

### Example 75

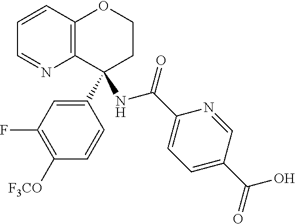 [SMILES: O=C(O)c1ccc(C(=O)N[C@]2(c3ccc(OC(F)(F)F)c(F)c3)CCOc3cccnc32)nc1]

### (S)-6-(4-(3-fluoro-4-(trifluoromethoxy)phenyl)-3,4-dihydro-2H-pyrano[3,2-b]pyridin-4-yl)carbamoyl)nicotinic acid

To a solution of (S)-methyl 64(4-((3-fluoro-4-(trifluoromethoxy)phenyl)-3,4-dihydro-2H-pyrano[3,2-b]pyridin-4-yl)carbamoyl)nicotinate (7.6 g, 15.47 mmol) and THF (30 mL):MeOH (10 mL), was added 5M NaOH (6 mL, 30.0 mmol) dropwise. The solution was stirred at room temperature. After 20 min, LC-MS indcated complete conversion. The reaction was poured into water (30 mL) and treated with 5N HCl (6 mL). The aqueous solution was extracted with EtOAc (3×25 mL). The combined organic layers were washed with brine, dried over MgSO₄, and concentrated in vacuo to give the title compound. MS (ESI, positive ion) m/z: 478.0 (MH+). ¹H NMR (400 MHz, DMSO-d₆) δ: 13.72 (br. s., 1H), 9.61 (s, 1H), 9.10 (dd, J=2.0, 0.8 Hz, 1H), 8.47 (dd, J=8.0, 2.2 Hz, 1H), 8.33 (dd, J=3.6, 2.2 Hz, 1H), 8.10-8.23 (m, 1H), 7.48-7.69 (m, 2H), 7.36-7.48 (m, 2H), 7.29 (dt, J=8.8, 1.1 Hz, 1H), 4.42 (dt, J=11.9, 4.1 Hz, 1H), 4.07 (td, J=11.3, 2.2 Hz, 1H), 3.50 (dt, J=12.2, 2.3 Hz, 1H), 2.95 (ddd, J=14.6, 10.9, 3.6 Hz, 1H).

### Example 76

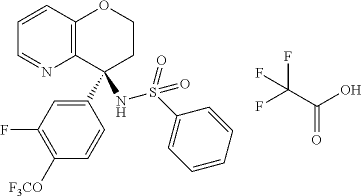

### (S)—N-(4-(3-fluoro-4-(trifluoromethoxy)phenyl)-3,4-dihydro-2H-pyrano[3,2-b]pyridin-4-yl)benzenesulfonamide 2,2,2-trifluoroacetate

To a solution of (S)-4-(3-fluoro-4-(trifluoromethoxy)phenyl)-3,4-dihydro-2H-pyrano[3,2-b]pyridin-4-amine hydrochloride (0.030 g, 0.082 mmol) in DCM (0.5 mL) was added benzenesulfonic chloride (0.012 mL, 0.099 mmol) and TEA (0.017 mL, 0.123 mmol). The resulting mixture was then stirred at room temperature for 18 h. The mixture was filtered and the filtrate was purified by preparative HPLC (0-100% MeCN 0.1% TFA/H2O 0.1% TFA) to give the title compound as a yellow solid. MS (ESI, positive ion) m/z: 469 (MH+). ¹H NMR (400 MHz, d₄-MeOH) δ: 8.10 (dd, J=4.5, 1.4 Hz, 1H), 7.56-7.65 (m, 2H), 7.43-7.51 (m, 1H), 7.25-7.40 (m, 4H), 7.20-7.25 (m, 1H), 7.10-7.19 (m, 2H), 4.19-4.46 (m, 1H), 3.87-4.10 (m, 1H), 2.70-2.98 (m, 2H).

### Example 77

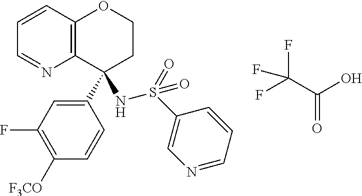 [0964] (S)—N-(4-(3-fluoro-4-(trifluoromethoxy)phenyl)-3,4-dihydro-2H-pyrano[3,2-b]pyridin-4-yl)pyridine-3-sulfonamide 2,2,2-trifluoroacetate. To a solution of (S)-4-(3-fluoro-4-(trifluoromethoxy)phenyl)-3,4-dihydro-2H-pyrano[3,2-b]pyridin-4-amine hydrochloride (0.030 g, 0.082 mmol) in DCM (0.5 mL) were added 3-pyridinesulfonylchloride HCl (0.015 mL, 0.099 mmol) and TEA (0.034 mL, 0.247 mmol). The resulting mixture was stirred at room temperature for 18 h. The mixture was filtered and the filtrate was purified by preparative HPLC (0-100% MeCN 0.1% TFA/H2O 0.1% TFA) to give the title compound as a yellow solid. MS (ESI, positive ion) m/z: 470 (MH+). ¹H NMR (400 MHz, d₄-MeOH) δ: 8.70 (d, J=1.8 Hz, 1H), 8.62 (dd, J=5.0, 1.5 Hz, 1H), 7.97-8.07 (m, 2H), 7.44 (dd, J=8.0, 4.9 Hz, 1H), 7.30-7.38 (m, 2H), 7.16-7.29 (m, 3H), 4.34-4.46 (m, 1H), 3.97-4.13 (m, 1H), 2.73-3.01 (m, 2H).

### Example 78

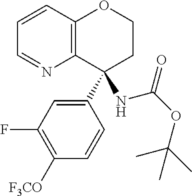 [SMILES: CC(C)(C)OC(=O)N[C@]1(c2ccc(OC(F)(F)F)c(F)c2)CCOc2cccnc21]

### (S)-tert-butyl (4-(3-fluoro-4-(trifluoromethoxy)phenyl)-3,4-dihydro-2H-pyrano[3,2-b]pyridin-4-yl)carbamate

To a solution of (S)-4-(3-fluoro-4-(trifluoromethoxy)phenyl)-3,4-dihydro-2H-pyrano[3,2-b]pyridin-4-amine HCl (41.8 mg, 0.115 mmol) and DCM (2 mL) were added TEA (0.032 mL, 0.229 mmol) and di-tert-butyl dicarbonate (0.032 mL, 0.138 mmol). After 72 h, the reaction was washed with water (5 mL) and concentrated in vacuo. The product thus obtained was adsorbed onto a plug of silica gel and chromatographed through a Redi-Sep® pre-packed silica gel column (4 g), eluting with DCM, to provide the title compound as a colorless oil. MS (ESI, positive ion) m/z: 429.0 (MH+). ¹H NMR (300 MHz, CDCl₃) δ: 8.22 (t, J=3.0 Hz, 1H), 7.15-7.25 (m, 4H), 7.04-7.14 (m, 1H), 6.32 (br. s., 1H), 4.34 (dt, J=11.7, 4.1 Hz, 1H), 4.00 (td, J=11.4, 2.4 Hz, 1H), 3.13 (d, J=14.6 Hz, 1H), 2.97 (ddd, J=14.7, 11.1, 3.8 Hz, 1H), 1.33-1.49 (m, 9H).

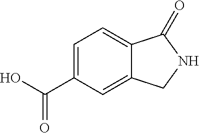 [SMILES: O=C(O)c1ccc2c(c1)CNC2=O]

### Intermediate 20

### 1-oxoisoindoline-5-carboxylic acid

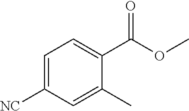

Step 1. methyl 4-cyano-2-methylbenzoate

To a stirred solution of methyl 4-bromo-2-methylbenzoate (2 g, 8.7 mmol) in DMF (20 mL), was added CuCN (1.9 g, 12.2 mmol). The reaction was then stirred at 175° C. for 6 h. The progress of the reaction was monitored by TLC (10% EtOAc in hexanes). The reaction was diluted with EtOAc (10 mL) and filtered through Celite® brand filtering agent. The filtrate was concentrated in vacuo and the material was purified by column chromatography (SiO₂, 0-50% EtOAc in hexanes) to give the title compound. MS (ESI pos. ion) m/z: 177.1 (MH+).

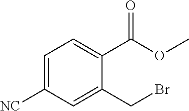

Step 2. methyl 2-(bromomethyl)-4-cyanobenzoate

To a stirred solution of methyl 4-cyano-2-methylbenzoate (1 g, 5.7 mmol) in CCl₄ (30 mL), was added NBS (1.4 g, 6.2 mmol), and catalytic AIBN. The reaction mixture was stirred for 48 h at 80° C. The reaction progress was monitored by TLC (10% EtOAc in hexanes). The reaction was cooled to room temperature and diluted with water (25 mL). The organic layer was separated and the aqueous layer extracted with EtOAc (2×50 mL). The combined organic layers were washed with brine (50 mL), dried over Na₂SO₄, concentrated, and purified by column chromatography silica (60-120 mesh silica with 80-100% EtOAc in petroleum ether) as eluent to give the title compound as a colorless solid. ¹H NMR (400 MHz, CDCl₃): δ 8.05 (d, J=8.0 Hz, 1H), 7.78 (s, 1H), 7.6 (d, J=8.0 Hz, 1H), 4.9 (s, 2H), 4.0 (s, 3H).

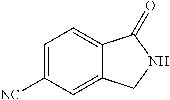 [SMILES: N#Cc1ccc2c(c1)CNC2=O]

Step 3. 1-oxoisoindoline-5-carbonitrile

To methyl 2-(bromomethyl)-4-cyanobenzoate (0.3 g, 1.18 mmol) in a sealed tube, was added 7N NH₃ in MeOH (10 mL). The tube was then sealed and the reaction stirred at 40° C. for 18 h. The reaction progress was monitored by TLC (90% EtOAc in hexanes). The reaction was filtered to give the title compound as a white solid. ¹H NMR (400 MHz, DMSO-d₆): δ 8.95 (s, 1H), 8.1 (s, 1H), 7.9 (d, J=8.0 Hz, 1H), 7.8 (d, J=8.0 Hz, 1H), 4.4 (s, 2H).

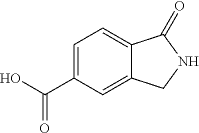 [SMILES: O=C(O)c1ccc2c(c1)CNC2=O]

Step 4. 1-oxoisoindoline-5-carboxylic acid

To a stirred solution of 1-oxoisoindoline-5-carbonitrile (0.15 g, 1.1 mmol) in H₂O (0.15 mL), were added H₂SO₄ (0.15 mL) and AcOH (0.15 mL). The reaction was then stirred at 100° C. for 24 h. The reaction progress was monitored by TLC (80% EtOAc in hexanes). The reaction was diluted with water (25 mL) and the organic layer separated. The aqueous layer was then extracted with EtOAc (2×50 mL). The combined organic layers were washed with brine (50 mL), dried over Na₂SO₄, and concentrated in vacuo to give the title compound as a colorless solid. MS (ESI pos. ion) m/z: 178.2 (MH-0.

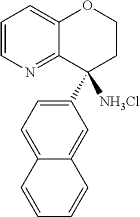

### Intermediate 21

### (S)-4-(naphthalen-2-yl)-3,4-dihydro-2H-pyrano[3,2-b]pyridin-4-amine hydrochloride

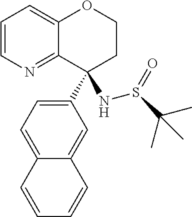 [SMILES: CC(C)(C)[S@](=O)N[C@]1(c2ccc3ccccc3c2)CCOc2cccnc21]

Step 1: (S)-2-methyl-N—((S)-4-(naphthalen-2-yl)-3,4-dihydro-2H-pyrano[3,2-b]pyridin-4-yl)propane-2-sulfinamide

To a solution of magnesium turnings (0.083 mL, 5.80 mmol) and iodine (10 mg) in 2-MeTHF (6 mL) under N₂ at room temperature was slowly added a solution of 2-bromonaphthalene (1.0 g, 4.83 mmol) in 2-MeTHF (4 mL). After addition, the mixture was stirred at room temperature for 18 h. The mixture was cooled to −78° C., and a solution of (S,E)-2-methyl-N-(2H-pyrano[3,2-b]pyridin-4(3H)-ylidene)propane-2-sulfinamide (0.490 g, 1.942 mmol) in 2-MeTHF (3 mL) was added dropwise. The mixture was then stirred at −78° C. for 1 h and at room temperature for 5 h. The mixture was quenched with saturated NH₄Cl (1.5 mL). The mixture was then extracted with EtOAc (2×5 mL). The combined organic extracts were dried over MgSO₄, concentrated, and purified by silica gel column chromatography using flash chromatography (30-60% EtOAc/hexanes) to give the title compound as a yellow solid. MS (ESI, positive ion) m/z: 381.0 (M+H)

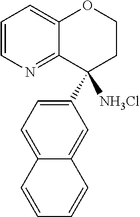

Step 2: (S)-4-(naphthalen-2-yl)-3,4-dihydro-2H-pyrano[3,2-b]pyridin-4-amine hydrochloride

To a solution of (S)-2-methyl-N—((S)-4-(naphthalen-2-yl)-3,4-dihydro-2H-pyrano[3,2-b]pyridin-4-yl)propane-2-sulfinamide (45 mg, 0.118 mmol) in EtOH (0.4 mL) was added 4M HCl in 1,4-dioxane (0.059 mL, 0.237 mmol). The mixture was stirred at room temperature for 1 h. The mixture was concentrated and dried in vacuo to give the title compound, which was used in the next step without further purification. MS (ESI, positive ion) m/z: 277.1 (M+H).

TABLE 4
Examples 79-81 were prepared via amide formation analogous to the procedures used to synthesize the compounds in Table 2.

| | | | | | MS |
|---|---|---|---|---|---|
| Ex # | Amine Intermediate | Acid Structure | Product Structure | Product Name | M + H |
| 79 | 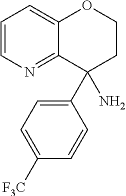 [SMILES: NC1(c2ccc(C(F)(F)F)cc2)CCOc2cccnc21] | 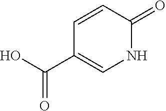 | 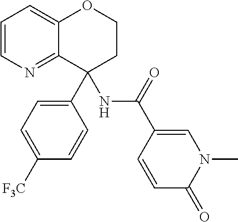 [SMILES: Cn1cc(C(=O)NC2(c3ccc(C(F)(F)F)cc3)CCOc3cccnc32)ccc1=O] | 1-methyl-6-oxo-N-(4-(4- (trifluoromethyl)phenyl)- 3,4-dihydro-2H- pyrano[3,2-b]pyridin-4- yl)-1,6-dihydropyridine- 3-carboxamide | 429.8 |
| 80 | 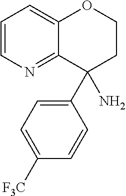 [SMILES: NC1(c2ccc(C(F)(F)F)cc2)CCOc2cccnc21] | 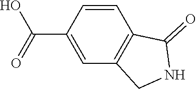 [SMILES: O=C(O)c1ccc2c(c1)CNC2=O] | 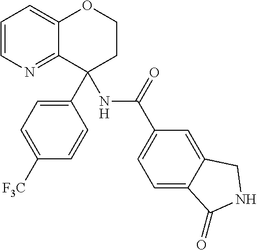 [SMILES: O=C(NC1(c2ccc(C(F)(F)F)cc2)CCOc2cccnc21)c1ccc2c(c1)CNC2=O] | 1-oxo-N-(4-(4- (trifluoromethyl)phenyl)- 3,4-dihydro-2H- pyrano[3,2-b]pyridin-4- yl)isoindoline-5- carboxamide | 454.3 |
| 81 | 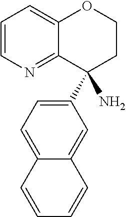 [SMILES: N[C@]1(c2ccc3ccccc3c2)CCOc2cccnc21] | 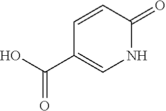 | 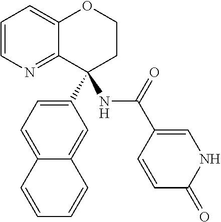 [SMILES: O=C(N[C@]1(c2ccc3ccccc3c2)CCOc2cccnc21)c1ccc(=O)[nH]c1] | (S)-N-(4-(naphthalen-2- yl)-3,4-dihydro-2H- pyrano[3,2-b]pyridin-4- yl)-6-oxo-1,6- dihydropyridine-3- carboxamide | 398.0 |

### General Urea Formation Procedure for Examples 82-105

Method A: To a solution of (S)-4-(3-fluoro-4-(trifluoromethoxy)phenyl)-3,4-dihydro-2H-pyrano[3,2-b]pyridin-4-amine hydrochloride (1 eq.) in DCM was added the desired isocyanate (1.2 eq) and DIPEA (1 eq). The resulting mixture was then stirred at room temperature for 18 h. Then, the mixture was concentrated in vacuo and the residue was dissolved in DMSO (1 mL). The solution mixture was then purified by preparative reverse phase HPLC (10-100% MeCN 0.1% TFA/H₂O 0.1% TFA) to give the title compound.

Method B: To a mixture of a carboxylic acid (1.2 eq.) and DIPEA (1.2 eq.) in 2-MeTHF was added diphenyl phosphorazidate (1.2 eq.). The resulting mixture was stirred at room temperature under N₂ for 4 h. A solution of (S)-4-(3-fluoro-4-(trifluoromethoxy)phenyl)-3,4-dihydro-2H-pyrano[3,2-b]pyridin-4-amine hydrochloride (1 eq.) and DIPEA (1.2 ea.) in 2-MeTHF was added, and the reaction was heated in an 80° C. oil bath for 18 h. The reaction was then cooled and purified by preparative reverse phase HPLC (10-100% MeCN, 0.1% TFA/H₂O, 0.1% TFA) to give the title compound.

Method C: A 0° C. solution of (S)-4-(3-fluoro-4-(trifluoromethoxy)phenyl)-3,4-dihydro-2H-pyrano[3,2-b]pyridin-4-amine hydrochloride (0.247 g, 0.677 mmol) in 1,2-dichloroethane (56.4 ml) was treated with DIPEA (0.589 ml, 3.385 mmol), followed by triphosgene (0.134 g, 0.452 mmol). The resulting mixture was allowed to warm to room temperature and stirred for 18 h. The reaction was recooled to 0° C. and treated with amine (5 eq); stirred at 0° C. for 5 min. and room temperature for 30 min. The reaction was then diluted with DCM and washed with saturated NaHCO₃ solution. The aqueous layer was extracted with DCM (1×). The combined organic layers were dried (MgSO₄), filtered, and concentrated in vacuo. Flash column chromatography (Silica gel, 0-30% EtOAc/Hexanes) afforded the title compound.

TABLE 5
Examples 82-105 prepared via urea formation analogous to general procedures described above.

| | | | Isocyanate, Acid | | | MS |
|---|---|---|---|---|---|---|
| Ex # | Method | Amine Intermediate | or Amine Structure | Product Structure | Product Name | M + H |
| 82 | B | 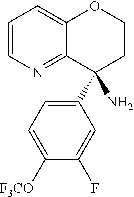 [SMILES: N[C@]1(c2ccc(OC(F)(F)F)c(F)c2)CCOc2cccnc21] | 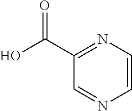 | 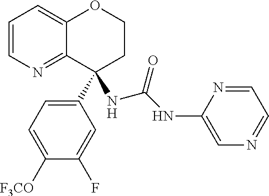 [SMILES: O=C(Nc1cnccn1)N[C@]1(c2ccc(OC(F)(F)F)c(F)c2)CCOc2cccnc21] | (S)-1-(4-(3- fluoro-4-(tri- fluorometh- oxy)phenyl)- 3,4-dihydro- 2H- pyrano[3,2- b]pyridin-4- yl)-3-(pyrazin- 2-yl)urea | 449.9 |
| 83 | B | 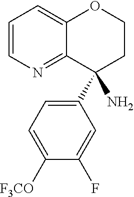 [SMILES: N[C@]1(c2ccc(OC(F)(F)F)c(F)c2)CCOc2cccnc21] | 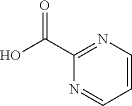 | 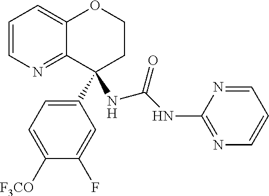 [SMILES: O=C(Nc1ncccn1)N[C@]1(c2ccc(OC(F)(F)F)c(F)c2)CCOc2cccnc21] | (S)-1-(4-(3- fluoro-4-(tri- fluorometh- oxy)phenyl)- 3,4-dihydro- 2H- pyrano[3,2- b]pyridin-4- yl)-3- (pyrimidin-2- yl)urea | 449.9 |
| 84 | B | 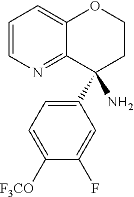 [SMILES: N[C@]1(c2ccc(OC(F)(F)F)c(F)c2)CCOc2cccnc21] | 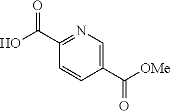 [SMILES: COC(=O)c1ccc(C(=O)O)nc1] | 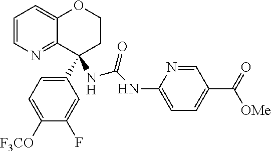 [SMILES: COC(=O)c1ccc(NC(=O)N[C@]2(c3ccc(OC(F)(F)F)c(F)c3)CCOc3cccnc32)nc1] | (S)-methyl 6- (3-(4-(3- fluoro-4-(tri- fluorometh- oxy)phenyl)- 3,4-dihydro- 2H- pyrano[3,2- b]pyridin-4- yl)ureido)nic- otinate | 506.9 |
| 85 | B | 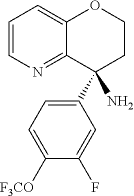 [SMILES: N[C@]1(c2ccc(OC(F)(F)F)c(F)c2)CCOc2cccnc21] | 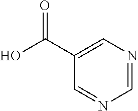 | 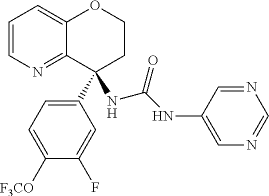 [SMILES: O=C(Nc1cncnc1)N[C@]1(c2ccc(OC(F)(F)F)c(F)c2)CCOc2cccnc21] | (S)-1-(4-(3- fluoro-4-(tri- fluorometh- oxy)phenyl)- 3,4-dihydro- 2H- pyrano[3,2- b]pyridin-4- yl)-3- (pyrimidin-5- yl)urea | 449.9 |
| 86 | B | 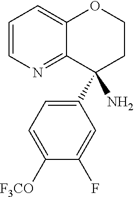 [SMILES: N[C@]1(c2ccc(OC(F)(F)F)c(F)c2)CCOc2cccnc21] | 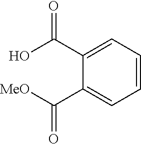 [SMILES: COC(=O)c1ccccc1C(=O)O] | 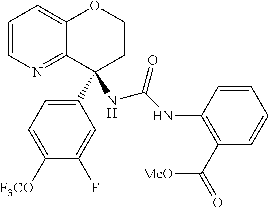 [SMILES: COC(=O)c1ccccc1NC(=O)N[C@]1(c2ccc(OC(F)(F)F)c(F)c2)CCOc2cccnc21] | (S)-methyl 2- (3-(4-(3- fluoro-4-(tri- fluorometh- oxy)phenyl)- 3,4-dihydro- 2H- pyrano[3,2- b]pyridin-4- yl)ureido)ben- zoate | 506.0 |
| 87 | B | 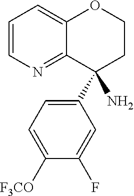 [SMILES: N[C@]1(c2ccc(OC(F)(F)F)c(F)c2)CCOc2cccnc21] | 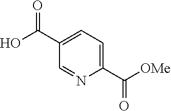 [SMILES: COC(=O)c1ccc(C(=O)O)cn1] | 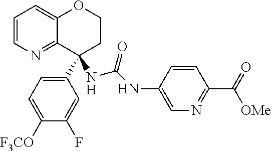 [SMILES: COC(=O)c1ccc(NC(=O)N[C@]2(c3ccc(OC(F)(F)F)c(F)c3)CCOc3cccnc32)cn1] | (S)-methyl 5- (3-(4-(3- fluoro-4-(tri- fluorometh- oxy)phenyl)- 3,4-dihydro- 2H- pyrano[3,2- b]pyridin-4- yl)ureido)pic- olinate | 506.9 |
| 88 | B | 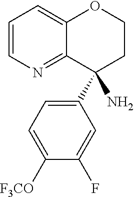 [SMILES: N[C@]1(c2ccc(OC(F)(F)F)c(F)c2)CCOc2cccnc21] | 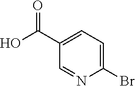 | 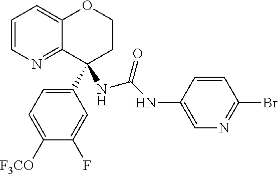 [SMILES: O=C(Nc1ccc(Br)nc1)N[C@]1(c2ccc(OC(F)(F)F)c(F)c2)CCOc2cccnc21] | (S)-1-(6- bromo- pyridin- 3-yl)-3-(4-(3- fluoro-4-(tri- fluorometh- oxy)phenyl)- 3,4-dihydro- 2H- pyrano[3,2- b]pyridin-4- yl)urea | 526.8, 528.8 |
| 89 | B | 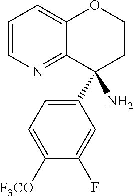 [SMILES: N[C@]1(c2ccc(OC(F)(F)F)c(F)c2)CCOc2cccnc21] | 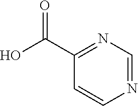 | 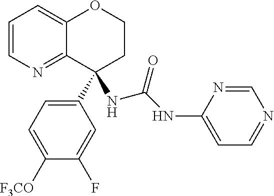 [SMILES: O=C(Nc1ccncn1)N[C@]1(c2ccc(OC(F)(F)F)c(F)c2)CCOc2cccnc21] | (S)-1-(4-(3- fluoro-4-(tri- fluorometh- oxy)phenyl)- 3,4-dihydro- 2H- pyrano[3,2- b]pyridin-4- yl)-3- (pyrimidin-4- yl)urea | 449.9 |
| 90 | B | 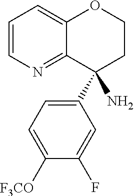 [SMILES: N[C@]1(c2ccc(OC(F)(F)F)c(F)c2)CCOc2cccnc21] | 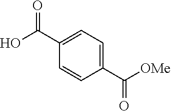 [SMILES: COC(=O)c1ccc(C(=O)O)cc1] | 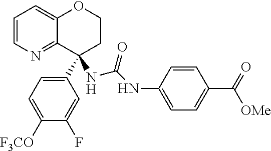 [SMILES: COC(=O)c1ccc(NC(=O)N[C@]2(c3ccc(OC(F)(F)F)c(F)c3)CCOc3cccnc32)cc1] | (S)-methyl 4- (3-(4-(3- fluoro-4-(tri- fluorometh- oxy)phenyl)- 3,4-dihydro- 2H- pyrano[3,2- b]pyridin-4- yl)ureido)ben- zoate | 506.0 |
| 91 | A | 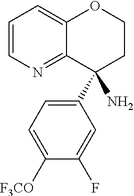 [SMILES: N[C@]1(c2ccc(OC(F)(F)F)c(F)c2)CCOc2cccnc21] | 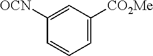 | 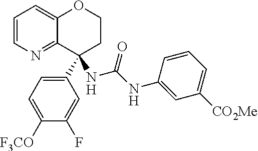 [SMILES: COC(=O)c1cccc(NC(=O)N[C@]2(c3ccc(OC(F)(F)F)c(F)c3)CCOc3cccnc32)c1] | (S)-methyl 3- (3-(4-(3- fluoro-4-(tri- fluorometh- oxy)phenyl)- 3,4-dihydro- 2H- pyrano[3,2- b]pyridin-4- yl)ureido)ben- zoate | 506.0 |
| 92 | B | 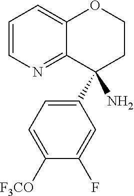 [SMILES: N[C@]1(c2ccc(OC(F)(F)F)c(F)c2)CCOc2cccnc21] | 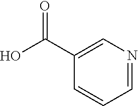 | 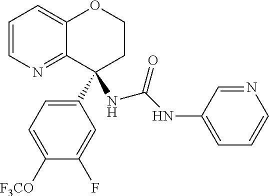 [SMILES: O=C(Nc1cccnc1)N[C@]1(c2ccc(OC(F)(F)F)c(F)c2)CCOc2cccnc21] | (S)-1-(4-(3- fluoro-4-(tri- fluorometh- oxy)phenyl)- 3,4-dihydro- 2H- pyrano[3,2- b]pyridin-4- yl)-3-(pyridin- 3-yl)urea | 449.0 |
| 93 | B | 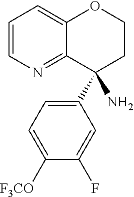 [SMILES: N[C@]1(c2ccc(OC(F)(F)F)c(F)c2)CCOc2cccnc21] | 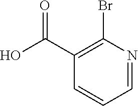 | 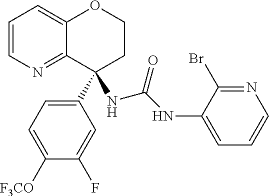 [SMILES: O=C(Nc1cccnc1Br)N[C@]1(c2ccc(OC(F)(F)F)c(F)c2)CCOc2cccnc21] | (S)-1-(2- bromo- pyridin- 3-yl)-3-(4-(3- fluoro-4-(tri- fluorometh- oxy)phenyl)- 3,4-dihydro- 2H- pyrano[3,2- b]pyridin-4- yl)urea | 527.0, 529.0 |
| 94 | B | 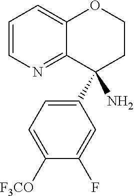 [SMILES: N[C@]1(c2ccc(OC(F)(F)F)c(F)c2)CCOc2cccnc21] | 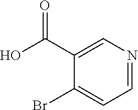 | 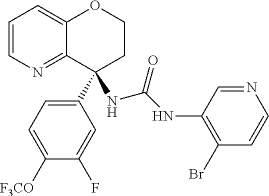 [SMILES: O=C(Nc1cnccc1Br)N[C@]1(c2ccc(OC(F)(F)F)c(F)c2)CCOc2cccnc21] | (S)-1-(4- bromo- pyridin-3- yl)-3-(4-(3- fluoro-4-(tri- fluorometh- oxy)phenyl)- 3,4-dihydro- 2H- pyrano[3,2- b]pyridin-4- yl)urea | 526.8, 528.8 |
| 95 | B | 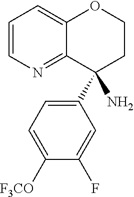 [SMILES: N[C@]1(c2ccc(OC(F)(F)F)c(F)c2)CCOc2cccnc21] | 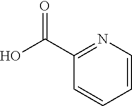 | 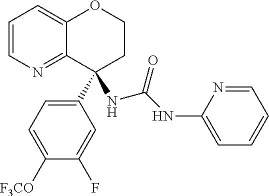 [SMILES: O=C(Nc1ccccn1)N[C@]1(c2ccc(OC(F)(F)F)c(F)c2)CCOc2cccnc21] | (S)-1-(4-(3- fluoro-4-(tri- fluorometh- oxy)phenyl)- 3,4-dihydro- 2H- pyrano[3,2- b]pyridin-4- yl)-3-(pyridin- 2-yl)urea | 449.0 |
| 96 | B | 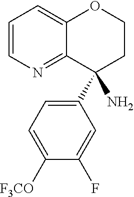 [SMILES: N[C@]1(c2ccc(OC(F)(F)F)c(F)c2)CCOc2cccnc21] | 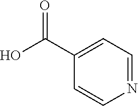 | 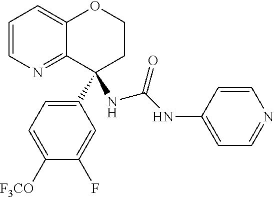 [SMILES: O=C(Nc1ccncc1)N[C@]1(c2ccc(OC(F)(F)F)c(F)c2)CCOc2cccnc21] | (S)-1-(4-(3- fluoro-4-(tri- fluorometh- oxy)phenyl)- 3,4-dihydro- 2H- pyrano[3,2- b]pyridin-4- yl)-3-(pyridin- 4-yl)urea | 449.0 |
| 97 | B | 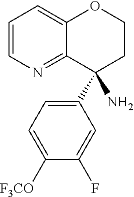 [SMILES: N[C@]1(c2ccc(OC(F)(F)F)c(F)c2)CCOc2cccnc21] | 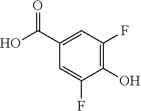 [SMILES: O=C(O)c1cc(F)c(O)c(F)c1] | 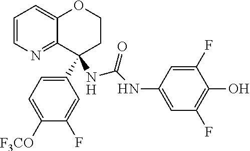 [SMILES: O=C(Nc1cc(F)c(O)c(F)c1)N[C@]1(c2ccc(OC(F)(F)F)c(F)c2)CCOc2cccnc21] | (S)-1-(3,5- difluoro-4- hydroxy- phenyl)- 3-(4-(3- fluoro-4-(tri- fluorometh- oxy)phenyl)- 3,4-dihydro- 2H- pyrano[3,2- b]pyridin-4- | 500.1 |
| | | | | | yl)urea | |
| 98 | C | 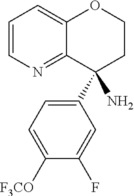 [SMILES: N[C@]1(c2ccc(OC(F)(F)F)c(F)c2)CCOc2cccnc21] | 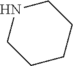 | 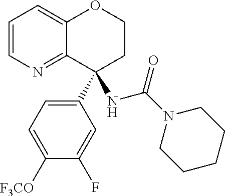 [SMILES: O=C(N[C@]1(c2ccc(OC(F)(F)F)c(F)c2)CCOc2cccnc21)N1CCCCC1] | (S)-N-(4-(3- fluoro-4-(tri- fluorometh- oxy)phenyl)- 3,4-dihydro- 2H- pyrano[3,2- b]pyridin-4- yl)piperidine- 1-carboxamide | 440.0 |
| 99 | C | 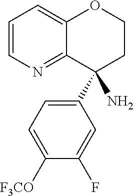 [SMILES: N[C@]1(c2ccc(OC(F)(F)F)c(F)c2)CCOc2cccnc21] | 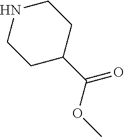 | 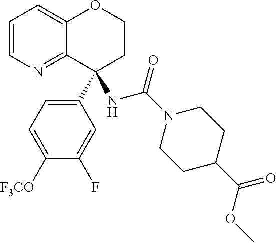 [SMILES: COC(=O)C1CCN(C(=O)N[C@]2(c3ccc(OC(F)(F)F)c(F)c3)CCOc3cccnc32)CC1] | (S)-methyl 1- ((4-(3- fluoro-4-(tri- fluorometh- oxy)phenyl)- 3,4-dihydro- 2H- pyrano[3,2- b]pyridin-4- yl)carbamo- yl)piperidine- 4-carboxylate | 498.0 |
| 100 | C | 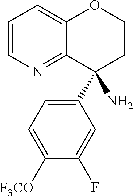 [SMILES: N[C@]1(c2ccc(OC(F)(F)F)c(F)c2)CCOc2cccnc21] | 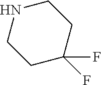 | 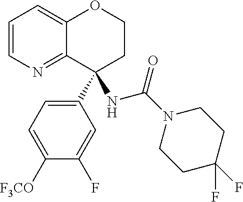 [SMILES: O=C(N[C@]1(c2ccc(OC(F)(F)F)c(F)c2)CCOc2cccnc21)N1CCC(F)(F)CC1] | (S)-4,4- difluoro-N- (4-(3- fluoro-4-(tri- fluorometh- oxy)phenyl- 3,4-dihydro- 2H- pyrano[3,2- b]pyridin-4- yl)piperidine- 1-carboxamide | 476.0 |
| 101 | C | 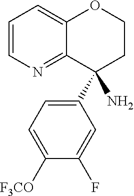 [SMILES: N[C@]1(c2ccc(OC(F)(F)F)c(F)c2)CCOc2cccnc21] | 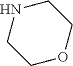 | 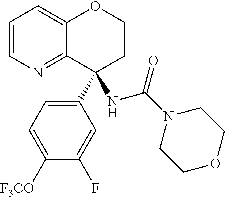 [SMILES: O=C(N[C@]1(c2ccc(OC(F)(F)F)c(F)c2)CCOc2cccnc21)N1CCOCC1] | (S)-N-(4-(3- fluoro-4-(tri- fluorometh- oxy)phenyl)- 3,4-dihydro- 2H- pyrano[3,2- b]pyridin-4- yl)morpho- line-4- carboxamide | 442.0 |
| 102 | C | 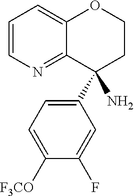 [SMILES: N[C@]1(c2ccc(OC(F)(F)F)c(F)c2)CCOc2cccnc21] | 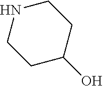 | 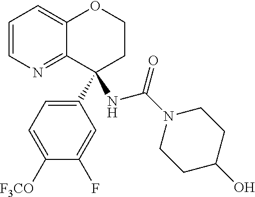 [SMILES: O=C(N[C@]1(c2ccc(OC(F)(F)F)c(F)c2)CCOc2cccnc21)N1CCC(O)CC1] | (S)-N-(4-(3- fluoro-4-(tri- fluorometh- oxy)phenyl)- 3,4-dihydro- 2H- pyrano[3,2- b]pyridin-4- yl)-4- hydroxypiper- idine-1- carboxamide | 456.0 |
| 103 | C | 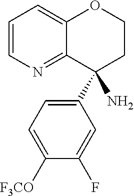 [SMILES: N[C@]1(c2ccc(OC(F)(F)F)c(F)c2)CCOc2cccnc21] | 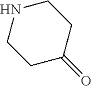 | 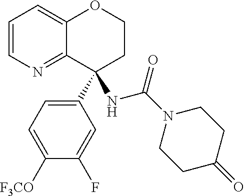 [SMILES: O=C1CCN(C(=O)N[C@]2(c3ccc(OC(F)(F)F)c(F)c3)CCOc3cccnc32)CC1] | (S)-N-(4-(3- fluoro-4-(tri- fluorometh- oxy)phenyl)- 3,4-dihydro- 2H- pyrano[3,2- b]pyridin-4- yl)-4- oxopiperidine- 1-carboxamide | 454.1 |
| 104 | C | 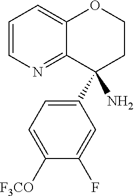 [SMILES: N[C@]1(c2ccc(OC(F)(F)F)c(F)c2)CCOc2cccnc21] | 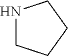 | 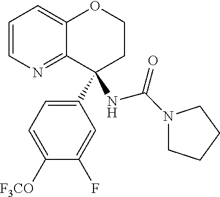 [SMILES: O=C(N[C@]1(c2ccc(OC(F)(F)F)c(F)c2)CCOc2cccnc21)N1CCCC1] | (S)-N-(4-(3- fluoro-4-(tri- fluorometh- oxy)phenyl)- 3,4-dihydro- 2H- pyrano[3,2- b]pyridin-4- yl)pyrrolidine- 1-carboxamide | 426.0 |
| 105 | C | 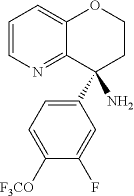 [SMILES: N[C@]1(c2ccc(OC(F)(F)F)c(F)c2)CCOc2cccnc21] | 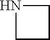 | 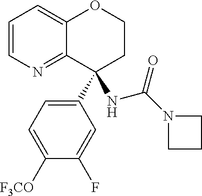 [SMILES: O=C(N[C@]1(c2ccc(OC(F)(F)F)c(F)c2)CCOc2cccnc21)N1CCC1] | (S)-N-(4-(3- fluoro-4-(tri- fluorometh- oxy)phenyl)- 3,4-dihydro- 2H- pyrano[3,2- b]pyridin-4- yl)azetidine- 1-carboxamide | 412.1 |

### Example 106

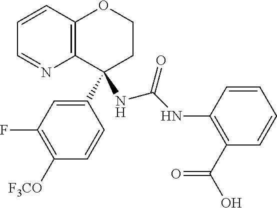 [SMILES: O=C(Nc1ccccc1C(=O)O)N[C@]1(c2ccc(OC(F)(F)F)c(F)c2)CCOc2cccnc21]

### (S)-2-(3-(4-(3-fluoro-4-(trifluoromethoxy)phenyl)-3,4-dihydro-2H-pyrano[3,2-b]pyridin-4-yl)ureido)benzoic acid

(S)-methyl 2-(3-(4-(3-fluoro-4-(trifluoro-methoxy)phenyl)-3,4-dihydro-2H-pyrano[3,2-b]pyridin-4-yl)ureido)benzoate (61 mg, 0.121 mmol) was dissolved in 2-MeTHF (1 mL). LiOH (2M in H₂O, 0.22 mL, 0.44 mmol) was added, and the resulting mixture was heated at 50° C. for 19 h. The reaction was neutralized with 1N HCl (0.44 mL), the solution diluted with H₂O and extracted with EtOAc (2×5 mL). The organic layers were combined, dried (MgSO₄), concentrated, and dried under high vacuum to give the title compound as a white solid. MS (ESI, positive ion) m/z: 491.9 (MH+). ¹H NMR (300 MHz, d₄-MeOH) δ: 8.18-8.28 (m, 1H), 8.14 (d, J=8.3 Hz, 1H), 8.00 (d, J=7.6 Hz, 1H), 7.53-7.65 (m, 1H), 7.28-7.53 (m, 4H), 7.17 (d, J=7.9 Hz, 1H), 7.00 (t, J=7.2 Hz, 1H), 4.37-4.54 (m, 1H), 4.17 (t, J=9.4 Hz, 1H), 3.12-3.26 (m, 1H), 2.55-2.76 (m, 1H).

### Example 107

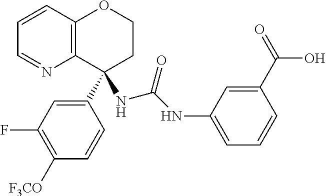 [SMILES: O=C(Nc1cccc(C(=O)O)c1)N[C@]1(c2ccc(OC(F)(F)F)c(F)c2)CCOc2cccnc21]

### (S)-3-(3-(4-(3-fluoro-4-(trifluoromethoxy)phenyl)-3,4-dihydro-2H-pyrano[3,2-b]pyridin-4-yl)ureido)benzoic acid

(S)-methyl 3-(3-(4-(3-fluoro-4-(trifluoro-methoxy)phenyl)-3,4-dihydro-2H-pyrano[3,2-b]pyridin-4-yl)ureido)benzoate (86.7 mg, 0.172 mmol) was dissoved in THF (1 mL). MeOH (0.4 mL) and LiOH (2M in H₂O, 0.69 mL, 1.38 mmol) were added, and the resulting mixture was stirred for 3 h at room temperature. The reaction was neutralized with 1N HCl (1.38 mL), the solution diluted with H₂O and extracted with EtOAc (2×5 mL). The organic layers were combined, dried (MgSO₄), concentrated, and dried under high vacuum to give the title compound as a white solid. MS (ESI, positive ion) m/z: 491.9 (MH+). ¹H NMR (300 MHz, d₄-MeOH) δ: 8.25 (dd, J=4.6, 1.5 Hz, 1H), 7.98 (t, J=1.8 Hz, 1H), 7.63 (dt, J=7.7, 1.3 Hz, 1H), 7.51-7.60 (m, 2H), 7.28-7.51 (m, 4H), 7.13-7.21 (m, 1H), 4.44 (ddd, J=11.8, 6.1, 3.4 Hz, 1H), 4.01-4.23 (m, 1H), 3.07-3.24 (m, 1H), 2.69-2.91 (m, 1H).

### Example 108

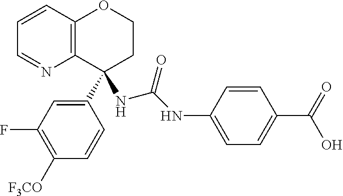 [SMILES: O=C(Nc1ccc(C(=O)O)cc1)N[C@]1(c2ccc(OC(F)(F)F)c(F)c2)CCOc2cccnc21]

### (S)-4-(3-(4-(3-fluoro-4-(trifluoromethoxy)phenyl)-3,4-dihydro-2H-pyrano[3,2-b]pyridin-4-yl)ureido)benzoic acid.

(5)-methyl 4-(3-(4-(3-fluoro-4-(trifluoro-methoxy)phenyl)-3,4-dihydro-2H-pyrano[3,2-b]pyridin-4-yl)ureido)benzoate (84.4 mg, 0.167 mmol) was dissoved in THF (1 mL) and MeOH (0.4 mL) and LiOH (2M in H₂O, 0.67 mL, 1.34 mmol) were added. The resulting mixture was then stirred for 2 h at room temperature. Additional LiOH (2M in H₂O, 4 eq, 0.67 mL, 1.34 mmol) was added, and the reaction was stirred for an additional 2 h. The reaction was neutralized with 1N HCl (2.68 mL), the solution diluted with H₂O and extracted with EtOAc (2×5 mL). The organic layers were combined, dried (MgSO₄), concentrated, and dried under high vacuum to give the title compound as a white solid. MS (ESI, positive ion) m/z: 491.9 (MH+). ¹H NMR (300 MHz, d₄-MeOH) δ: 8.26 (dd, J=4.8, 1.5 Hz, 1H), 7.82-7.98 (m, 2H), 7.58 (dd, J=8.5, 1.5 Hz, 1H), 7.51 (dd, J=8.5, 4.7 Hz, 1H), 7.33-7.47 (m, 4H), 7.13-7.21 (m, 1H), 4.45 (ddd, J=11.8, 6.0, 3.4 Hz, 1H), 4.14 (ddd, J=11.9, 9.5, 2.7 Hz, 1H), 3.07-3.25 (m, 1H), 2.70-2.90 (m, 1H).

### Example 109

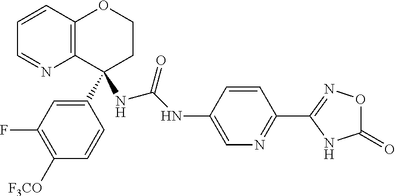 [SMILES: O=C(Nc1ccc(-c2noc(=O)[nH]2)nc1)N[C@]1(c2ccc(OC(F)(F)F)c(F)c2)CCOc2cccnc21]

### (S)-1-(4-(3-fluoro-4-(trifluoromethoxy)phenyl)-3,4-dihydro-2H-pyrano[3,2-b]pyridin-4-yl)-3-(6-(5-oxo-4,5-dihydro-1,2,4-oxadiazol-3-yl)pyridin-3-yl)urea

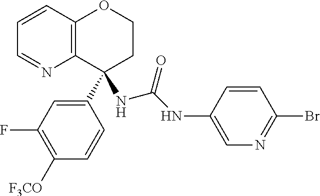 [SMILES: O=C(Nc1ccc(Br)nc1)N[C@]1(c2ccc(OC(F)(F)F)c(F)c2)CCOc2cccnc21]

Step 1. (S)-1-(6-bromopyridin-3-yl)-3-(4-(3-fluoro-4-(trifluoromethoxy)phenyl)-3,4-dihydro-2H-pyrano[3,2-b]pyridin-4-yl)urea

To a mixture of 6-bromonicotinic acid (332 mg, 1.645 mmol), and DIPEA (0.287 mL, 1.645 mmol) in 2-MeTHF (10 mL) was added diphenyl phosphorazidate (0.367 mL, 1.645 mmol). The resulting mixture was stirred at room temperature under N₂ for 30 min. A solution of (S)-4-(3-fluoro-4-(trifluoromethoxy)phenyl)-3,4-dihydro-2H-pyrano[3,2-b]pyridin-4-amine hydrochloride (500 mg, 1.371 mmol) and DIPEA (0.287 mL, 1.645 mmol) was added, and the reaction was heated in an 80° C. oil bath for 26 h. The reaction was cooled, poured into saturated aqueous NaHCO₃ (30 mL) and extracted with EtOAc (3×25 mL). The organic layers were combined, dried (MgSO₄), and concentrated. Purification by flash chromatography (40 g SiO₂, 25-100% EtOAc in hexanes) gave the title compound as a light pink foam. MS (ESI, positive ion) m/z: 526.8, 528.8 (MH+).

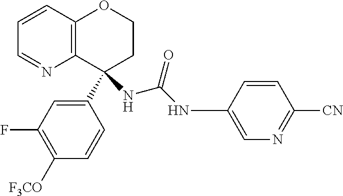 [SMILES: N#Cc1ccc(NC(=O)N[C@]2(c3ccc(OC(F)(F)F)c(F)c3)CCOc3cccnc32)cn1]

Step 2. (S)-1-(6-cyanopyridin-3-yl)-3-(4-(3-fluoro-4-(trifluoromethoxy)-phenyl)-3,4-dihydro-2H-pyrano[3,2-b]pyridin-4-yl)urea

A 250 mL RBF containing (S)-1-(6-bromopyridin-3-yl)-3-(4-(3-fluoro-4-(trifluoromethoxy)-phenyl)-3,4-dihydro-2H-pyrano[3,2-b]pyridin-4-yl)urea (300 mg, 0.569 mmol), tetrakis(triphenylphosphine)palladium (0) (26.3 mg, 0.023 mmol), and zinc cyanide (0.042 mL, 0.654 mmol) was flushed with N₂ (3×), and DMF (3 mL) was added. The reaction was immersed in a preheated 80° C. oil bath and stirred under N₂ for 18 h. The reaction was cooled to room temperature, water was added, and the solution was extracted with EtOAc (3×10 mL). The organic layers were combined, dried (MgSO₄), and the solvent removed in vacuo. The residue was purified by flash chromatography (40 g SiO₂, 0-100% EtOAc in hexanes) to give the title compound as a light white foam. MS (ESI, positive ion) m/z: 473.9 (MH+).

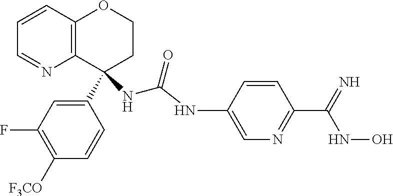 [SMILES: N=C(NO)c1ccc(NC(=O)N[C@]2(c3ccc(OC(F)(F)F)c(F)c3)CCOc3cccnc32)cn1]

Step 3. (S)-5-(3-(4-(3-fluoro-4-(trifluoromethoxy)phenyl)-3,4-dihydro-2H-pyrano[3,2-b]pyridin-4-yl)ureido)-N-hydroxypicolinimidamide

To a 20 mL vial containing (S)-1-(6-cyanopyridin-3-yl)-3-(4-(3-fluoro-4-(trifluoromethoxy)-phenyl)-3,4-dihydro-2H-pyrano[3,2-b]pyridin-4-yl)urea (65 mg, 0.137 mmol), hydroxylamine hydrochloride (10.50 mg, 0.151 mmol), and NaHCO₃ (13.84 mg, 0.165 mmol), was added MeOH (2 mL). The vial was sealed and the suspension was heated to 50° C. in a heating block and stirred for 21 h. The mixture was diluted with H₂O (10 mL), and the solids were filtered off. The solids were washed with H₂O (3 mL) and dried under vacuum to give the title compound as a white solid.

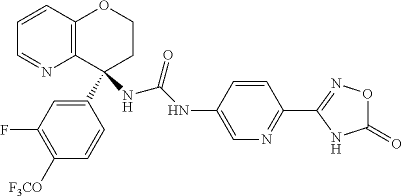 [SMILES: O=C(Nc1ccc(-c2noc(=O)[nH]2)nc1)N[C@]1(c2ccc(OC(F)(F)F)c(F)c2)CCOc2cccnc21]

Step 4. (S)-1-(4-(3-fluoro-4-(trifluoromethoxy)phenyl)-3,4-dihydro-2H-pyrano[3,2-b]pyridin-4-yl)-3-(6-(5-oxo-4,5-dihydro-1,2,4-oxadiazol-3-yl)pyridin-3-yl)urea

A suspension of (S)-5-(3-(4-(3-fluoro-4-(trifluoromethoxy)-phenyl)-3,4-dihydro-2H-pyrano[3,2-b]pyridin-4-yl)ureido)-N-hydroxypicolin-imidamide (50 mg, 0.099 mmol) and 1,1′-carbonyldiimidazole (22.3 mg, 0.137 mmol) in 1,4-dioxane (1 mL) was heated in a sealed vial in a 100° C. heating block for 2.5 h. The reaction was cooled, the reaction was diluted with DMF (1 mL), and the product was purified by reverse phase HPLC (Gemini Axia 50×250 mm C18, 10-100% CH₃CN, 0.1% TFA/H₂O, 0.1% TFA). The product containing fractions were combined, and the solvent was removed by lyophilization. The resulting solid was dissolved in MeOH and passed through a StratoSpheres® SPE PL-HCO₃ (6 mL, 0.36 mmol) cartridge with MeOH (5 mL). Concentration of the filtrates afforded the title compound as a white solid. MS (ESI, positive ion) m/z: 533.0 (MH+). ¹H NMR (300 MHz, d₄-MeOH) δ: 9.10 (d, J=1.6 Hz, 1H), 8.30-8.42 (m, 2H), 8.22 (d, J=8.2 Hz, 1H), 8.03 (td, J=8.5, 1.3 Hz, 1H), 7.87 (dd, J=8.6, 5.3 Hz, 1H), 7.41-7.54 (m, 2H), 7.24 (d, J=8.6 Hz, 1H), 4.56-4.70 (m, 1H), 4.35-4.45 (m, 1H), 3.02-3.18 (m, 1H), 2.79 (ddd, J=14.7, 8.2, 3.2 Hz, 1H).

### Example 110

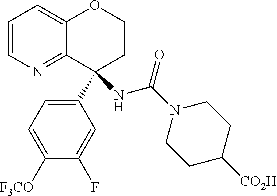 [SMILES: O=C(O)C1CCN(C(=O)N[C@]2(c3ccc(OC(F)(F)F)c(F)c3)CCOc3cccnc32)CC1]

### (S)-1-(4-(3-fluoro-4-(trifluoromethoxy)phenyl)-3,4-dihydro-2H-pyrano[3,2-b]pyridin-4-yl)carbamoyl)piperidine-4-carboxylic acid

To a solution of (S)-methyl 1-((4-(3-fluoro-4-(trifluoromethoxy)phenyl)-3,4-dihydro-2H-pyrano[3,2-b]pyridin-4-yl)carbamoyl)piperidine-4-carboxylate (Example 99, 0.16 g, 0.322 mmol) and THF (1.6 ml):water (0.4 ml), was added LiOH (1N in H₂O, 0.643 ml, 0.643 mmol). The solution was stirred at room temperature for 2 h. The reaction was neutralized with 1N HCl and then concentrated in vacuo. Purification by reverse phase preparative HPLC afforded the title compound as a white solid. MS (ESI, positive ion) m/z: 484.0 (MH+). ¹H NMR (400 MHz, DMSO-d₆) δ: 12.15 (br. s., 1H), 8.10 (dd, J=3.91, 1.96 Hz, 1H), 7.39-7.48 (m, 2H), 7.20-7.28 (m, 2H), 7.13 (dd, J=8.71, 1.27 Hz, 1H), 6.93 (s, 1H), 4.29 (ddd, J=11.10, 7.58, 3.03 Hz, 1H), 4.07 (ddd, J=11.25, 8.22, 2.84 Hz, 1H), 3.82 (d, J=13.11 Hz, 2H), 3.13 (ddd, J=14.28, 8.22, 3.13 Hz, 1H), 2.68-2.84 (m, 3H), 2.39 (tt, J=11.13, 3.94 Hz, 1H), 1.70-1.80 (m, 2H), 1.35-1.47 (m, 2H).

### Example 111

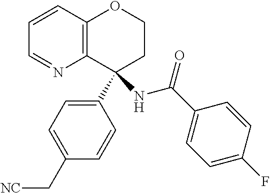 [SMILES: N#CCc1ccc([C@@]2(NC(=O)c3ccc(F)cc3)CCOc3cccnc32)cc1]

### (S)—N-(4-(4-(cyanomethyl)phenyl)-3,4-dihydro-2H-pyrano[3,2-b]pyridin-4-yl)-4-fluorobenzamide

A vial containing (S)—N-(4-(4-bromophenyl)-3,4-dihydro-2H-pyrano[3,2-b]pyridin-4-yl)-4-fluorobenzamide (50 mg, 0.117 mmol), K₃PO₄ (74.5 mg, 0.351 mmol), bis(di-tert-butyl(4-dimethyl-aminophenyl)phosphine)dichloropalladium(II) (8.29 mg, 0.012 mmol) and 4-(4,4,5,5-tetramethyl-1,3,2-dioxaborolan-2-yl)isoxazole (34.2 mg, 0.176 mmol) was flushed with Ar (3×). 2-MeTHF (2 mL) and water (0.1 mL) were added via syringe. The vial was sealed and the mixture was stirred in an 80° C. oil bath for 19 h. The reaction was then cooled to room temperature and filtered through Celite® brand filter agent (2×2 mL, EtOAc rinse). The filtrate was concentrated in vacuo, and the residue purified by flash chromatography (12 g SiO₂, 0-50% EtOAc in hexanes) to give the title compound as a white solid. MS (ESI, positive ion) m/z: 388.2 (MH+). ¹H NMR (300 MHz, DMSO-d₆) δ: 8.72 (s, 1H), 8.12 (dd, J=4.0, 2.0 Hz, 1H), 7.79-7.96 (m, 2H), 7.13-7.40 (m, 8H), 4.22-4.43 (m, 1H), 4.00 (s, 2H), 3.93-4.07 (m, 1H), 3.19-3.28 (m, 1H), 2.82 (ddd, J=14.1, 6.1, 2.5 Hz, 1H).

### Example 112

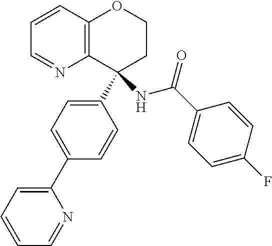 [SMILES: O=C(N[C@]1(c2ccc(-c3ccccn3)cc2)CCOc2cccnc21)c1ccc(F)cc1]

### (S)-4-fluoro-N-(4-(4-(pyridin-2-yl)phenyl)-3,4-dihydro-2H-pyrano[3,2-b]pyridin-4-yl)benzamide

A microwave vial containing (S)—N-(4-(4-bromophenyl)-3,4-dihydro-2H-pyrano[3,2-b]pyridin-4-yl)-4-fluorobenzamide (54 mg, 0.126 mmol), K₃PO₄ (134 mg, 0.632 mmol), chloro(2-dicyclohexyl-phosphino-2′,4′,6′-tri-1-propyl-1,1′-biphenyl)[2-(2-aminoethyl)-phenyl]palladium (II) methyl-t-butylether adduct (4.7 mg, 0.0063 mmol), diacetoxycopper (11.5 mg, 0.063 mmol), and 2-pyridinylboronic acid MIDA ester (44.4 mg, 0.190 mmol) was flushed with N₂ (3×). DMF (1 mL) and diethyl amine (13 μL, 0.126 mmol) were added via syringe. The vial was sealed, and stirred in an 100° C. oil bath for 5 h. The reaction was then cooled to room temperature, and filtered through Celite® brand filter agent, (2×2 mL, EtOAc rinse). The filtrate was concentrated in vacuo, and the residue was purified by flash chromatography (12 g SiO₂, 0-50% EtOAc in hexanes) to give the title compound as a white solid. MS (ESI, positive ion) m/z: 426.0 (MH+). ¹H NMR (300 MHz, DMSO-d₆) δ: 8.77 (s, 1H), 8.65 (d, J=4.8 Hz, 1H), 8.15 (dd, J=4.0, 1.8 Hz, 1H), 7.96-8.08 (m, J=8.5 Hz, 2H), 7.77-7.96 (m, 4H), 7.37-7.48 (m, J=8.5 Hz, 2H), 7.15-7.37 (m, 5H), 4.26-4.43 (m, 1H), 4.04 (t, J=9.3 Hz, 1H), 3.26-3.36 (m, 1H), 2.79-2.96 (m, 1H).

### General Boronic Acid Coupling Procedure for Examples 113-151

To a vial containing boronic acid (1.5 equiv.) was added K₃PO₄ (3.0 equiv.) and bis(di-tert-butyl(4-dimethylaminophenyl)phosphine)dichloro palladium (II) (0.1 equiv.). The vial was sealed and flushed with N₂ (3×). A solution of (S)—N-(4-(4-bromophenyl)-3,4-dihydro-2H-pyrano[3,2-b]pyridin-4-yl)-4-fluorobenzamide (1.0 equiv.) in (16:1 2-MeTHF:H₂O) was added via syringe, and the vial was sealed and stirred in an 80° C. oil bath overnight. The reaction was cooled to room temperature and filtered through a frit (1×2 mL, EtOAc rinse). The filtrate was concentrated and purified by silica gel flash chromatography or preparative reverse phase HPLC to give the title compound.

### Representative Boronic Acid Coupling Procedure

### Example 113

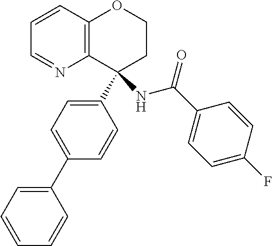 [SMILES: O=C(N[C@]1(c2ccc(-c3ccccc3)cc2)CCOc2cccnc21)c1ccc(F)cc1]

### (S)—N-(4-([1,1′-biphenyl]-4-yl)-3,4-dihydro-2H-pyrano[3,2-b]pyridin-4-yl)-4-fluorobenzamide

To a microwave vial containing (S)—N-(4-(4-bromophenyl)-3,4-dihydro-2H-pyrano[3,2-b]pyridin-4-yl)-4-fluorobenzamide (100 mg, 0.234 mmol), K₃PO₄ (149 mg, 0.702 mmol), and boronic acid (1.5 equiv.) was added K₃PO₄ (3.0 equiv.), bis(di-tert-butyl(4-dimethylamino-phenyl)-phosphine)dichloro palladium (II) (16.57 mg, 0.023 mmol) and phenylboronic acid (42.8 mg, 0.351 mmol). Ar (3×) was flushed through the vial. 2-MeTHF (2 mL) and water (0.1 mL) were then added via syringe. The vial was sealed, and the mixture was stirred in an 80° C. oil bath for 22 h. The reaction mixture was then cooled to room temperature, and filtered through Celite® brand filter agent, (2×2 mL, EtOAc rinse). The filtrates were concentrated in vacuo, and the residue was purified by flash chromatography (12 g SiO₂, 0-50% EtOAc in hexanes) to give the title compound as a white solid. MS (ESI, positive ion) m/z: 425.2 (MH+). ¹H NMR (300 MHz, MeOH-d₄) δ: 8.76 (s, 1H), 8.16 (dd, J=4.1, 1.9 Hz, 1H), 7.81-7.99 (m, 2H), 7.54-7.71 (m, 4H), 7.34-7.52 (m, 4H), 7.20-7.34 (m, 4H), 4.36 (ddd, J=11.4, 5.5, 3.4 Hz, 1H), 3.95-4.13 (m, 1H), 3.22-3.42 (m, 1H), 2.80-2.95 (m, 1H).

TABLE 6
Examples 114-151 prepared via boronic acid coupling analogous to general procedures described above.

| | Boronic Acid | Product | Product | MS |
|---|---|---|---|---|
| Ex # | Structure | Structure | Name | M + H |
| 114 | 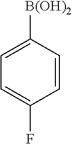 | 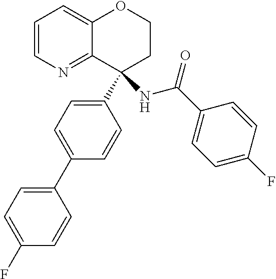 [SMILES: O=C(N[C@]1(c2ccc(-c3ccc(F)cc3)cc2)CCOc2cccnc21)c1ccc(F)cc1] | (S)-4-fluoro-N- (4-(4′-fluoro- [1,1′-biphenyl]- 4-yl)-3,4- dihydro-2H- pyrano[3,2- b]pyridin-4- yl)benzamide | 443.2 |
| 115 | 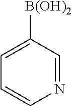 | 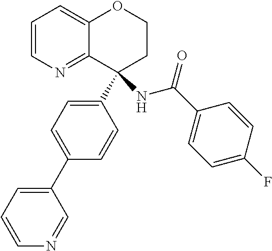 [SMILES: O=C(N[C@]1(c2ccc(-c3cccnc3)cc2)CCOc2cccnc21)c1ccc(F)cc1] | (S)-4-fluoro-N- (4-(4-(pyridin- 3-yl)phenyl)- 3,4-dihydro-2H- pyrano[3,2- b]pyridin-4- yl)benzamide | 426.2 |
| 116 | 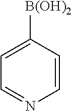 | 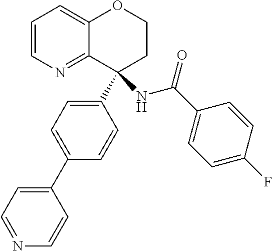 [SMILES: O=C(N[C@]1(c2ccc(-c3ccncc3)cc2)CCOc2cccnc21)c1ccc(F)cc1] | (S)-4-fluoro-N- (4-(4-(pyridin- 4-yl)phenyl)- 3,4-dihydro-2H- pyrano[3,2- b]pyridin-4- yl)benzamide | 426.0 |
| 117 | 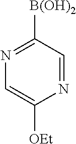 [SMILES: CCOc1cnc(B(O)O)cn1] | 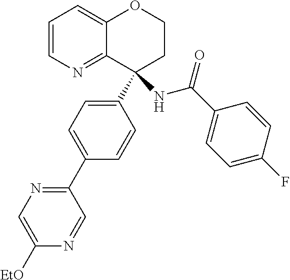 [SMILES: CCOc1cnc(-c2ccc([C@@]3(NC(=O)c4ccc(F)cc4)CCOc4cccnc43)cc2)cn1] | (S)-N-(4-(4-(5- ethoxypyrazin- 2-yl)phenyl)- 3,4-dihydro-2H- pyrano[3,2-b] pyridin-4-yl)-4- fluorobenzamide | 471.0 |
| 118 | 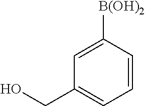 [SMILES: OCc1cccc(B(O)O)c1] | 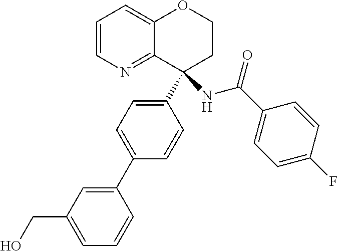 [SMILES: O=C(N[C@]1(c2ccc(-c3cccc(CO)c3)cc2)CCOc2cccnc21)c1ccc(F)cc1] | (S)-4-fluoro-N- (4-(3′- (hydroxymethyl)- [1,1′- biphenyl]-4-yl)- 3,4-dihydro-2H- pyrano[3,2- b]pyridin-4- yl)benzamide | 455.2 |
| 119 | 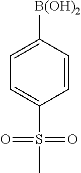 | 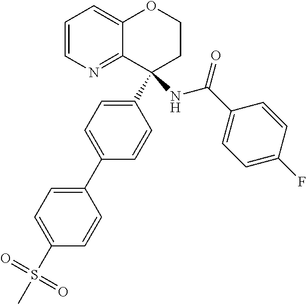 [SMILES: CS(=O)(=O)c1ccc(-c2ccc([C@@]3(NC(=O)c4ccc(F)cc4)CCOc4cccnc43)cc2)cc1] | (S)-4-fluoro-N- (4-(4′- (methylsulfonyl)- [1,1′- biphenyl]-4-yl)- 3,4-dihydro-2H- pyrano[3,2- b]pyridin-4- yl)benzamide | 503.2 |
| 120 | 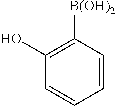 | 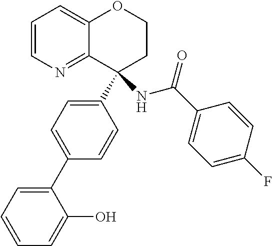 [SMILES: O=C(N[C@]1(c2ccc(-c3ccccc3O)cc2)CCOc2cccnc21)c1ccc(F)cc1] | (S)-4-fluoro-N- (4-(2′-hydroxy- [1,1′-biphenyl]- 4-yl)-3,4- dihydro-2H- pyrano[3,2- b]pyridin-4- yl)benzamide | 441.4 |
| 121 | 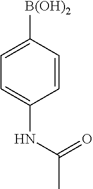 | 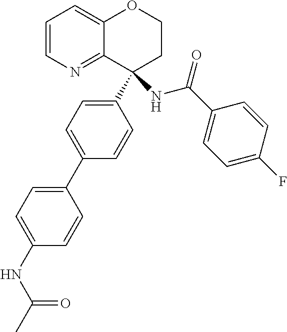 [SMILES: CC(=O)Nc1ccc(-c2ccc([C@@]3(NC(=O)c4ccc(F)cc4)CCOc4cccnc43)cc2)cc1] | (S)-N-(4-(4′- acetamido-[1,1′- biphenyl]-4-yl)- 3,4-dihydro-2H- pyrano[3,2-b] pyridin-4-yl)- 4-fluorobenzamide | 482.2 |
| 122 | 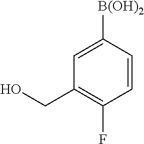 [SMILES: OCc1cc(B(O)O)ccc1F] | 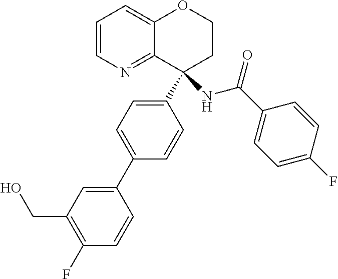 [SMILES: O=C(N[C@]1(c2ccc(-c3ccc(F)c(CO)c3)cc2)CCOc2cccnc21)c1ccc(F)cc1] | (S)-4-fluoro-N- (4-(4′-fluoro-3′- (hydroxymethyl)- [1,1- biphenyl]-4-yl)- 3,4-dihydro-2H- pyrano[3,2- b]pyridin-4- yl)benzamide | 473.2 |
| 123 | 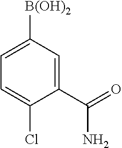 [SMILES: NC(=O)c1cc(B(O)O)ccc1Cl] | 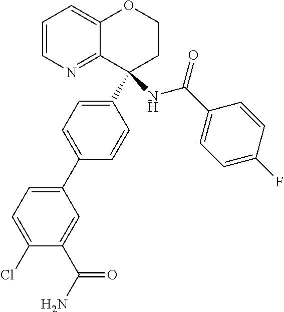 [SMILES: NC(=O)c1cc(-c2ccc([C@@]3(NC(=O)c4ccc(F)cc4)CCOc4cccnc43)cc2)ccc1Cl] | (S)-4-chloro-4′- (4-(4- fluorobenzamido)- 3,4-dihydro- 2H-pyrano[3,2- b]pyridin-4-yl)- [1,1′-biphenyl]- 3-carboxamide | 502.3 |
| 124 | 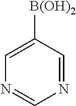 | 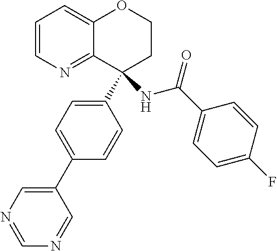 [SMILES: O=C(N[C@]1(c2ccc(-c3cncnc3)cc2)CCOc2cccnc21)c1ccc(F)cc1] | (S)-4-fluoro-N- (4-(4- (pyrimidin-5- yl)phenyl)-3,4- dihydro-2H- pyrano[3,2- b]pyridin-4- yl)benzamide | 427.3 |
| 125 | 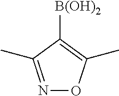 | 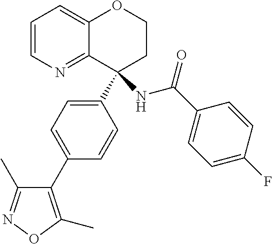 [SMILES: Cc1noc(C)c1-c1ccc([C@@]2(NC(=O)c3ccc(F)cc3)CCOc3cccnc32)cc1] | (S)-N-(4-(4- (3,5- dimethylisoxazol- 4-yl)phenyl)- 3,4-dihydro-2H- pyrano[3,2- b]pyridin-4-yl)- 4-fluorobenzamide | 444.4 |
| 126 | 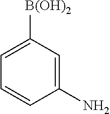 | 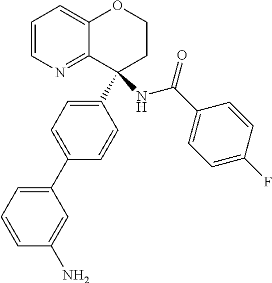 [SMILES: Nc1cccc(-c2ccc([C@@]3(NC(=O)c4ccc(F)cc4)CCOc4cccnc43)cc2)c1] | (S)-N-(4-(3′- amino-[1,1′- biphenyl]-4-yl)- 3,4-dihydro-2H- pyrano[3,2- b]pyridin-4-yl)- 4-fluorobenzamide | 440.2 |
| 127 | 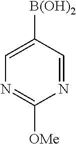 [SMILES: COc1ncc(B(O)O)cn1] | 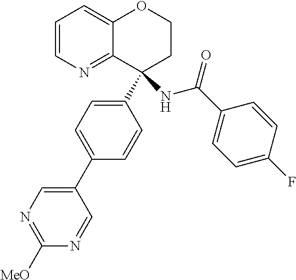 [SMILES: COc1ncc(-c2ccc([C@@]3(NC(=O)c4ccc(F)cc4)CCOc4cccnc43)cc2)cn1] | (S)-4-fluoro-N- (4-(4-(2- methoxypyrimidin- 5-yl)phenyl)-3,4- dihydro-2H- pyrano[3,2- b]pyridin-4- yl)benzamide | 457.3 |
| 128 | 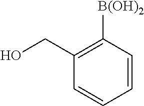 [SMILES: OCc1ccccc1B(O)O] | 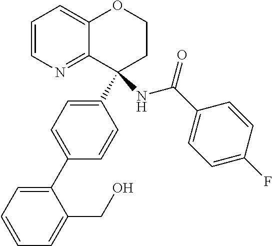 [SMILES: O=C(N[C@]1(c2ccc(-c3ccccc3CO)cc2)CCOc2cccnc21)c1ccc(F)cc1] | (S)-4-fluoro-N- (4-(2′- (hydroxymethyl)- [1,1′- biphenyl]-4-yl)- 3,4-dihydro-2H- pyrano[3,2- b]pyridin-4- yl)benzamide | 455.2 |
| 129 | 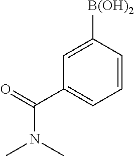 | 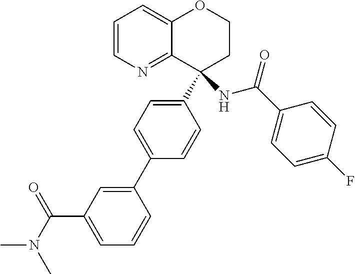 [SMILES: CN(C)C(=O)c1cccc(-c2ccc([C@@]3(NC(=O)c4ccc(F)cc4)CCOc4cccnc43)cc2)c1] | (S)-4′-(4-(4- fluorobenzamido)- 3,4-dihydro- 2H-pyrano[3,2- b]pyridin-4-yl)- N,N-dimethyl- [1,1′-biphenyl]- 3-carboxamide | 496.6 |
| 130 | 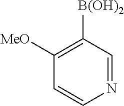 [SMILES: COc1ccncc1B(O)O] | 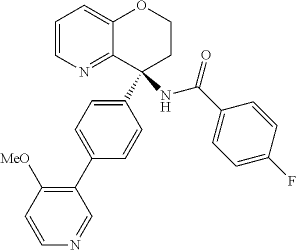 [SMILES: COc1ccncc1-c1ccc([C@@]2(NC(=O)c3ccc(F)cc3)CCOc3cccnc32)cc1] | (S)-4-fluoro-N- (4-(4-(4- methoxypyridin- 3-yl)phenyl)- 3,4-dihydro-2H- pyrano[3,2- b]pyridin-4- yl)benzamide | 456.4 |
| 131 | 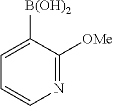 [SMILES: COc1ncccc1B(O)O] | 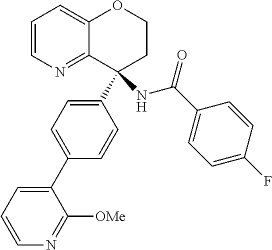 [SMILES: COc1ncccc1-c1ccc([C@@]2(NC(=O)c3ccc(F)cc3)CCOc3cccnc32)cc1] | (S)-4-fluoro-N- (4-(4-(2- methoxypyridin- 3-yl)phenyl)- 3,4-dihydro-2H- pyrano[3,2- b]pyridin-4- yl)benzamide | 456.4 |
| 132 | 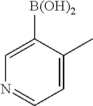 | 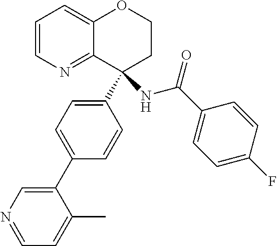 [SMILES: Cc1ccncc1-c1ccc([C@@]2(NC(=O)c3ccc(F)cc3)CCOc3cccnc32)cc1] | (S)-4-fluoro-N- (4-(4-(4- methylpyridin- 3-yl)phenyl)- 3,4-dihydro-2H- pyrano[3,2- b]pyridin-4- yl)benzamide | 440.2 |
| 133 | 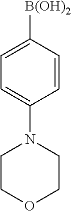 [SMILES: OB(O)c1ccc(N2CCOCC2)cc1] | 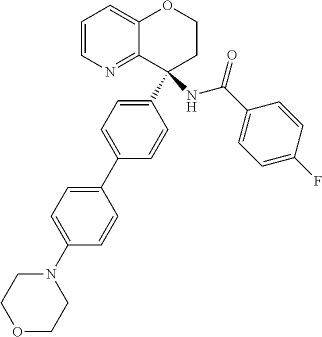 [SMILES: O=C(N[C@]1(c2ccc(-c3ccc(N4CCOCC4)cc3)cc2)CCOc2cccnc21)c1ccc(F)cc1] | (S)-4-fluoro-N- (4-(4′- morpholino- [1,1′-biphenyl]- 4-yl)-3,4- dihydro-2H- pyrano[3,2- b]pyridin-4- yl)benzamide | 510.4 |
| 134 | 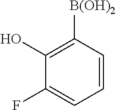 [SMILES: OB(O)c1cccc(F)c1O] | 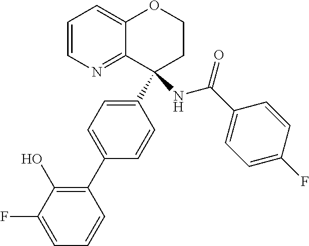 [SMILES: O=C(N[C@]1(c2ccc(-c3cccc(F)c3O)cc2)CCOc2cccnc21)c1ccc(F)cc1] | (S)-4-fluoro-N- (4-(3′-fluoro-2′- hydroxy-[1,1′- biphenyl]-4-yl)- 3,4-dihydro-2H- pyrano[3,2- b]pyridin-4- yl)benzamide | 459.4 |
| 135 | 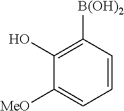 [SMILES: COc1cccc(B(O)O)c1O] | 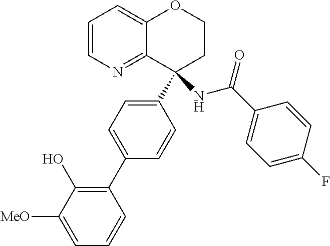 [SMILES: COc1cccc(-c2ccc([C@@]3(NC(=O)c4ccc(F)cc4)CCOc4cccnc43)cc2)c1O] | (S)-4-fluoro-N- (4-(2′-hydroxy- 3′-methoxy- [1,1′-biphenyl]- 4-yl)-3,4- dihydro-2H- pyrano[3,2- b]pyridin-4- yl)benzamide | 471.4 |
| 136 | 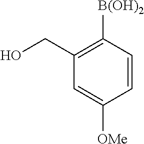 [SMILES: COc1ccc(B(O)O)c(CO)c1] | 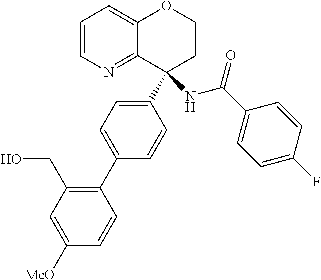 [SMILES: COc1ccc(-c2ccc([C@@]3(NC(=O)c4ccc(F)cc4)CCOc4cccnc43)cc2)c(CO)c1] | (S)-4-fluoro-N- (4-(2′- (hydroxymethyl)- 4′-methoxy- [1,1′-biphenyl]- 4-yl)-3,4- dihydro-2H- pyrano[3,2- b]pyridin-4- yl)benzamide | 485.5 |
| 137 | 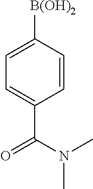 | 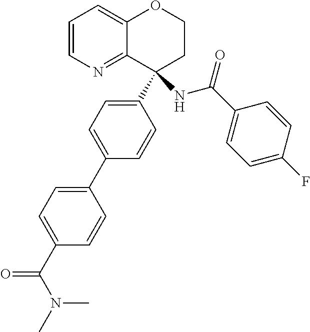 [SMILES: CN(C)C(=O)c1ccc(-c2ccc([C@@]3(NC(=O)c4ccc(F)cc4)CCOc4cccnc43)cc2)cc1] | (S)-4′-(4-(4- fluorobcnzamido)- 3,4-dihydro- 2H-pyrano[3,2- b]pyridin-4-yl)- N,N-dimethyl- [1,1′-biphenyl]- 4-carboxamide | 496.6 |
| 138 | 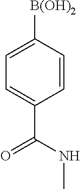 | 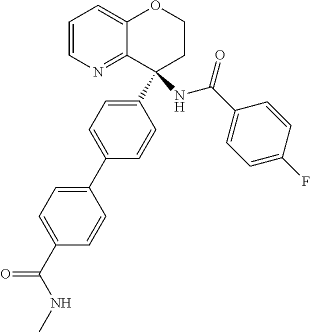 [SMILES: CNC(=O)c1ccc(-c2ccc([C@@]3(NC(=O)c4ccc(F)cc4)CCOc4cccnc43)cc2)cc1] | (S)-4′-(4-(4- fluorobenzamido)- 3,4-dihydro- 2H-pyrano[3,2- b]pyridin-4-yl)- N-methyl-[1,1′- biphenyl]-4- carboxamide | 482.2 |
| 139 | 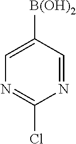 | 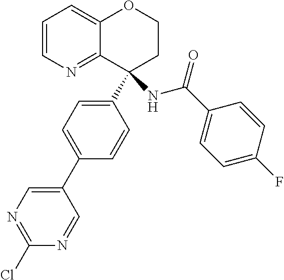 [SMILES: O=C(N[C@]1(c2ccc(-c3cnc(Cl)nc3)cc2)CCOc2cccnc21)c1ccc(F)cc1] | (S)-N-(4-(4-(2- chloropyrirnidin- 5-yl)phenyl)- 3,4-dihydro-2H- pyrano[3,2- b]pyridin-4-yl)- 4-fluorobenzamide | 461.2 |
| 140 | 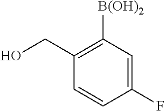 [SMILES: OCc1ccc(F)cc1B(O)O] | 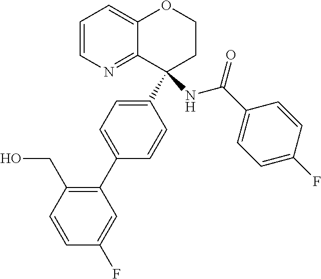 [SMILES: O=C(N[C@]1(c2ccc(-c3cc(F)ccc3CO)cc2)CCOc2cccnc21)c1ccc(F)cc1] | (S)-4-fluoro-N- (4-(5′-fluoro-2′- (hydroxymethyl)- [1,1′- biphenyl]-4-yl)- 3,4-dihydro-2H- pyrano[3,2- b]pyridin-4- yl)benzamide | 473.2 |
| 141 | 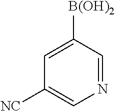 [SMILES: N#Cc1cncc(B(O)O)c1] | 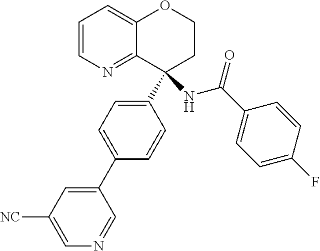 [SMILES: N#Cc1cncc(-c2ccc([C@@]3(NC(=O)c4ccc(F)cc4)CCOc4cccnc43)cc2)c1] | (S)-N-(4-(4-(5- cyanopyridin-3- yl)phenyl)-3,4- dihydro-2H- pyrano[3,2- b]pyridin-4-yl)- 4-fluorobenzamide | 451.3 |
| 142 | 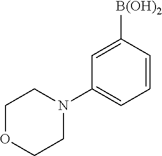 [SMILES: OB(O)c1cccc(N2CCOCC2)c1] | 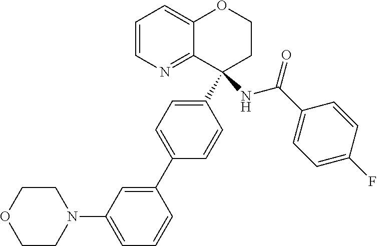 [SMILES: O=C(N[C@]1(c2ccc(-c3cccc(N4CCOCC4)c3)cc2)CCOc2cccnc21)c1ccc(F)cc1] | (S)-4-fluoro-N- (4-(3′- morpholino- [1,1′-biphenyl]- 4-yl)-3,4- dihydro-2H- pyrano[3,2- b]pyridin-4- yl)benzamide | 510.4 |
| 143 | 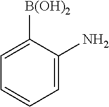 | 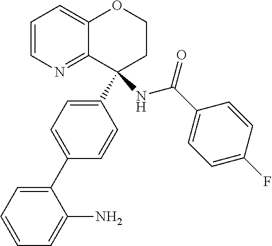 [SMILES: Nc1ccccc1-c1ccc([C@@]2(NC(=O)c3ccc(F)cc3)CCOc3cccnc32)cc1] | (S)-N-(4-(2′- amino-[1,1′- biphenyl]-4-yl)- 3,4-dihydro-2H- pyrano[3,2- b]pyridin-4-yl)- 4-fluorobenzamide | 440.2 |
| 144 | 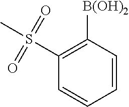 | 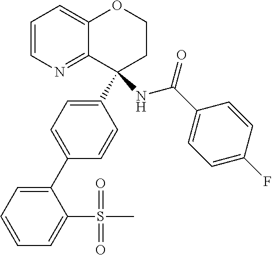 [SMILES: CS(=O)(=O)c1ccccc1-c1ccc([C@@]2(NC(=O)c3ccc(F)cc3)CCOc3cccnc32)cc1] | (S)-4-fluoro-N- (4-(2′- (methylsulfonyl)- [1,1′- biphenyl]-4-yl)- 3,4-dihydro-2H- pyrano[3,2- b]pyridin-4- yl)benzamide | 503.2 |
| 145 | 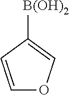 | 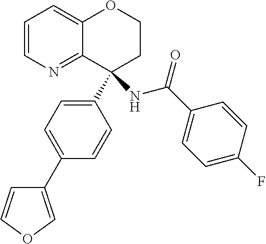 [SMILES: O=C(N[C@]1(c2ccc(-c3ccoc3)cc2)CCOc2cccnc21)c1ccc(F)cc1] | (S)-4-fluoro-N- (4-(4-(furan-3- yl)phenyl)-3,4- dihydro-2H- pyrano[3,2- b]pyridin-4- yl)benzamide | 415.3 |
| 146 | 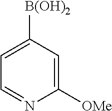 [SMILES: COc1cc(B(O)O)ccn1] | 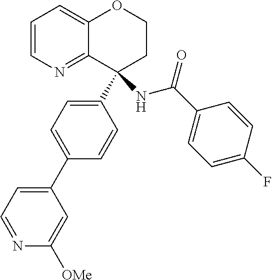 [SMILES: COc1cc(-c2ccc([C@@]3(NC(=O)c4ccc(F)cc4)CCOc4cccnc43)cc2)ccn1] | (S)-4-fluoro-N- (4-(4-(2- methoxypyridin- 4-yl)phenyl)- 3,4-dihydro-2H- pyrano[3,2- b]pyridin-4- yl)benzamide | 456.1 |
| 147 | 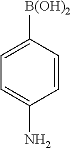 | 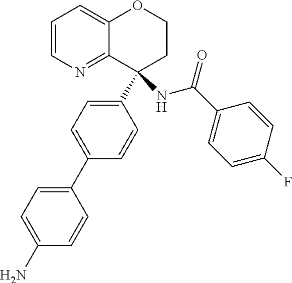 [SMILES: Nc1ccc(-c2ccc([C@@]3(NC(=O)c4ccc(F)cc4)CCOc4cccnc43)cc2)cc1] | (S)-N-(4-(4′- amino-[1,1′- biphenyl]-4-yl)- 3,4-dihydro-2H- pyrano[3,2- b]pyridin-4-yl)- 4-fluorobenzamide | 440.2 |
| 148 | 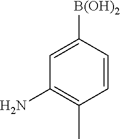 | 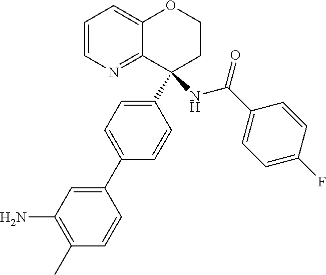 [SMILES: Cc1ccc(-c2ccc([C@@]3(NC(=O)c4ccc(F)cc4)CCOc4cccnc43)cc2)cc1N] | (S)-N-(4-(3′- amino-4′- methyl-[1,1′- biphenyl]-4-yl)- 3,4-dihydro-2H- pyrano[3,2- b]pyridin-4-yl)- 4-fluorobenzamide | 454.3 |
| 149 | 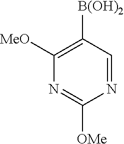 [SMILES: COc1ncc(B(O)O)c(OC)n1] | 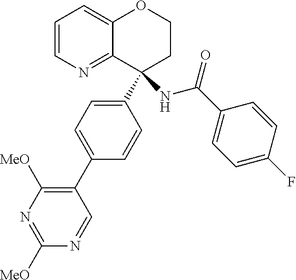 [SMILES: COc1ncc(-c2ccc([C@@]3(NC(=O)c4ccc(F)cc4)CCOc4cccnc43)cc2)c(OC)n1] | (S)-N-(4-(4- (2,4- dimethoxypyrimidin- 5-yl)phenyl)-3,4- dihydro-2H- pyrano[3,2- b]pyridin-4-yl)- 4-fluorobenzamide | 487.3 |
| 150 | 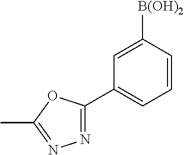 | 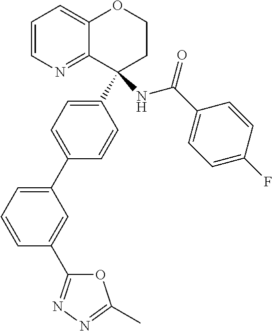 [SMILES: Cc1nnc(-c2cccc(-c3ccc([C@@]4(NC(=O)c5ccc(F)cc5)CCOc5cccnc54)cc3)c2)o1] | (S)-4-fluoro-N- (4-(3′-(5- methyl-1,3,4- oxadiazol-2-yl)- [1,1′-biphenyl]- 4-yl)-3,4- dihydro-2H- pyrano[3,2- b]pyridin-4- yl)benzamide | 507.1 |
| 151 | 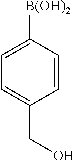 [SMILES: OCc1ccc(B(O)O)cc1] | 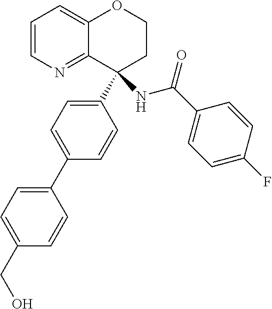 [SMILES: O=C(N[C@]1(c2ccc(-c3ccc(CO)cc3)cc2)CCOc2cccnc21)c1ccc(F)cc1] | (S)-4-fluoro-N- (4-(4′- (hydroxymethyl)- [1,1′- biphenyl]-4-yl)- 3,4-dihydro-2H- pyrano[3,2- b]pyridin-4- yl)benzamide | 455.2 |

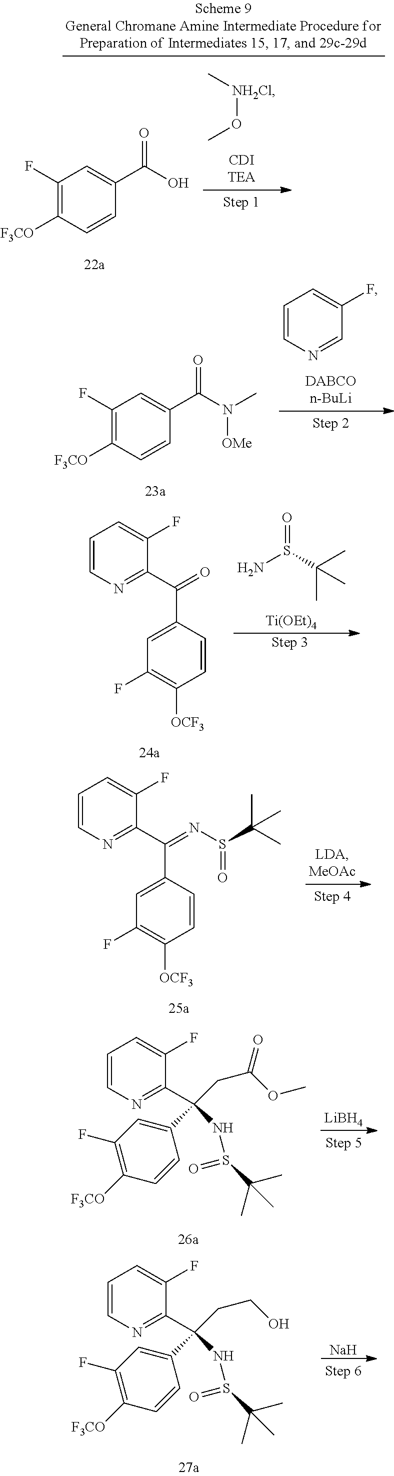

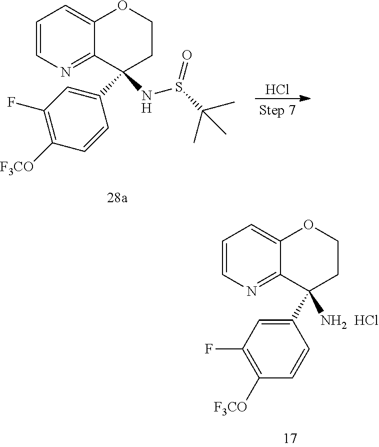

### Intermediate 23a

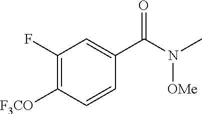 [SMILES: CON(C)C(=O)c1ccc(OC(F)(F)F)c(F)c1]

Step 1: 3-fluoro-N-methoxy-N-methyl-4-(trifluoromethoxy)benzamide

To a stirred solution of 3-fluoro-4-(trifluoromethoxy)benzoic acid (588 g, 2.65 mol) in DCM (2.9 L) at 0° C. was added 1,1′-carbonyldiimidazole (468 g, 2.88 mol) lotwise over a period of 20 min. After complete addition, the reaction mixture was warmed to room temperature and stirred for 3 h. The reaction was recooled to 0° C. and TEA (318 g, 3.15 mol) and N,O-dimethylhydroxylamine hydrochloride (281 g, 2.88 mol) were added successively. The reaction mixture was warmed to room temperature, and stirred for 24 h. The reaction was partially concentrated under reduced pressure. The concentrate was diluted with DCM (1 L) and water (1 L). The aqueous phase was extracted with DCM (2×2 L) and the combined organic layers were washed with brine (1×500 mL), dried over Na₂SO₄, filtered, and concentrated to afford the title compound as a brown liquid which was used without any further purification in the next step. ¹H NMR (300 MHz, DMSO-d₆): δ 7.79-7.63 (m, 2H), 7.57 (dd, J=0.9, 8.4 Hz, 1H), 3.56 (s, 3H), 3.27 (s, 3H).

TABLE 7
Intermediates 23b-23d were prepared via amide coupling analogous to the procedure described above

| Intermediate | Intermediate | |
|---|---|---|
| 22a-22d | 23a-23d | Analytical Data |
| 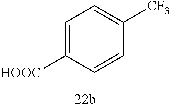 [SMILES: O=C(O)c1ccc(C(F)(F)F)cc1] | 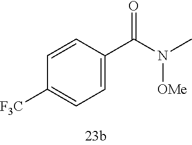 [SMILES: CON(C)C(=O)c1ccc(C(F)(F)F)cc1] | MS (ESI, positive ion) m/z: 233.9 (MH+). ¹H NMR (300 MHz, DMSO-d₆): δ 7.84 (dd, J = 8.40, 14.1 Hz, 4H), 3.21 (s, 3H), 3.48 (s, 3H). |
| 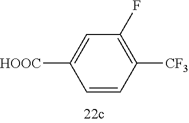 [SMILES: O=C(O)c1ccc(C(F)(F)F)c(F)c1] | 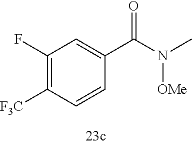 | MS (ESI, positive ion) m/z: 244.0 (MH+) |
| 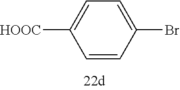 | 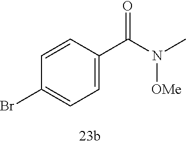 | MS (ESI, positive ion) m/z: 245.6 (MH+). ¹H NMR (300MHz, DMSO-d₆): δ 7.59-7.52 (m, 4H), 3.52 (s, 3H), 3.50 (s, 3H). |

### Intermediate 24a

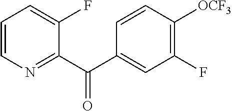 [SMILES: O=C(c1ccc(OC(F)(F)F)c(F)c1)c1ncccc1F]

Step 2. (3-fluoro-4-(trifluoromethoxy)phenyl)(3-fluoropyridin-2-yl)methanone

To a stirred solution of DABCO (336.5 g, 3.05 mol) in anhydrous diethyl ether (11.39 L) at −25° C. was added n-BuLi (2.5 M in n-hexane, 1.2 L, 3.0 mol). The mixture was stirred between −25° C. and −10° C. for 45 min. and then cooled to −70° C. To the above solution, was added 3-fluoropyridine (265 g, 2.7 mol) dropwise. The reaction was stirred between −70° C. and −60° C. for 1.5 h before 3-fluoro-N-methoxy-N-methyl-4-(trifluoromethoxy)benzamide (364 g, 1.36 mol) was added. After 3 h, approx. 30% of the starting material was observed (by TLC). Again 0.5 eq of the anion generated (same as above) was added to the reaction mass for complete conversion of starting material. After 1 h stirring at −70° C., water (1 L) was added, and the mixture was warmed to ambient temperature. The layers were separated and the aqueous layer was extracted with EtOAc (2×2 L). The combined organic layers were washed with brine and dried over Na₂SO₄. After removal of solvent, the residue was purified by silica gel (60-120 mesh) chromatography using gradient 15% EtOAc in hexanes to give the title compound as pale yellow oil. MS (ESI, positive ion) m/z: 304.1 (MH+).

TABLE 8
Intermediates 24b-24d were prepared via anion addition analogous to the procedure described above

| Intermediate | Intermediate | |
|---|---|---|
| 23b-d | 24b-d | Analytical Data for 24b-d |
| 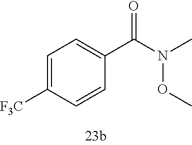 [SMILES: CON(C)C(=O)c1ccc(C(F)(F)F)cc1] | 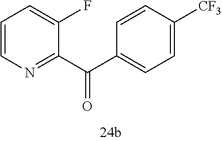 [SMILES: O=C(c1ccc(C(F)(F)F)cc1)c1ncccc1F] | ¹H NMR (300 MHz, CDCl₃): δ 8.55- 8.52 (m, 1H), 7.83-7.67 (m, 3H), 7.62-7.54 (m, 2H). |
| 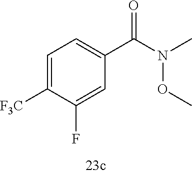 [SMILES: CON(C)C(=O)c1ccc(C(F)(F)F)c(F)c1] | 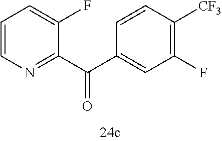 [SMILES: O=C(c1ccc(C(F)(F)F)c(F)c1)c1ncccc1F] | ¹H NMR (400 MHz, DMSO-d₆): δ 8.56-8.54 (m, 1H), 8.04-7.91 (m, 3H), 7.84-7.78 (m, 2H). |
| 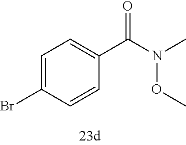 | 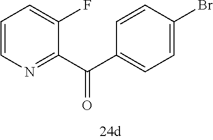 [SMILES: O=C(c1ccc(Br)cc1)c1ncccc1F] | MS (ESI, positive ion) m/z: 281.6 (MH+). ¹H NMR (300 MHz, DMSO- d₆): δ 8.56-8.54 (dd, 1H), 8.50-7.98 (dd, 1H), 7.77-7.73 (m, 5H). |

### Intermediate 25a

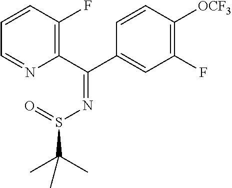 [SMILES: CC(C)(C)[S@@](=O)/N=C(/c1ccc(OC(F)(F)F)c(F)c1)c1ncccc1F]

Step 3: (R,Z)—N-((3-fluoro-4-(trifluoromethoxy)phenyl)(3-fluoropyridin-2-yl)methylene)-2-methylpropane-2-sulfinamide

To a solution of (3-fluoro-4-(trifluoromethoxy)phenyl)(3-fluoropyridin-2-yl)methanone (1 kg, 3.3 mol) in THF in a 100 mL RB flask, were added (3 L), titanium (IV) ethoxide (1.12 kg, 4.95 mol) and (R)-2-methylpropane-2-sulfinamide (399 g, 3.3 mol) in one lot. The resulting mixture was stirred for 15 h at 75° C. After completion of reaction, the reaction mixture was allowed to cool to ambient temperature and was diluted with water (1 L). The reaction mixture was filtered through Celite® brand filter agent and concentrated under reduced pressure. The thick liquid thus obtained was purified by silica gel column chromatography using 30-70% EtOAc in petroleum ether as eluent to afford the title compound as a brown oil. ¹H NMR (300 MHz, DMSO-d₆): δ 8.52 (dd, J=1.2, 2.7 Hz, 1H), 7.86 (dt, J=1.2, 9.6 Hz, 1H), 7.71-7.61 (m, 3H), 7.38 (dd, J=1.8, 9.0 Hz, 1H), 1.25 (s, 9H).

TABLE 9
Intermediates 25b-25d were prepared via imine formation analogous to the procedure described above

| Intermediate | Intermediate | |
|---|---|---|
| 24b-4d | 25b-25d | Analytical Data for 25b-d |
| 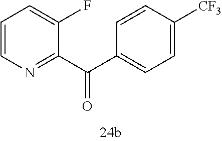 [SMILES: O=C(c1ccc(C(F)(F)F)cc1)c1ncccc1F] | 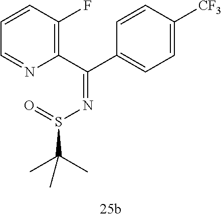 [SMILES: CC(C)(C)[S@@](=O)/N=C(/c1ccc(C(F)(F)F)cc1)c1ncccc1F] | ¹H NMR (300 MHz, CDCl₃): δ 8.53-8.50 (m, 1H), 7.73-7.70 (m, 2H), 7.66-7.63 (m, 2H), 7.53-7.42 (m, 2H), 1.35 (s, 9H). |
| 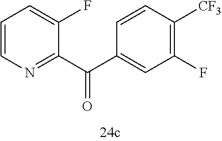 [SMILES: O=C(c1ccc(C(F)(F)F)c(F)c1)c1ncccc1F] | 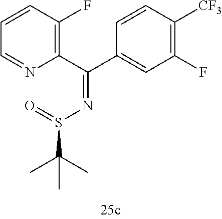 [SMILES: CC(C)(C)[S@@](=O)/N=C(/c1ccc(C(F)(F)F)c(F)c1)c1ncccc1F] | ¹H NMR (300 MHz, DMSO-d₆): δ 8.53 (d, J = 3.6 Hz, 1H), 7.91-7.85 (m, 3H), 7.75-7.72 (d, J = 8.4 Hz, 1H), 7.67-7.62 (m, 1H), 1.27 (s, 9H). |
| 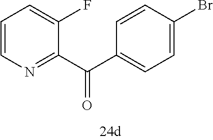 [SMILES: O=C(c1ccc(Br)cc1)c1ncccc1F] | 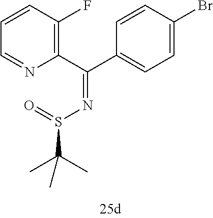 [SMILES: CC(C)(C)[S@@](=O)/N=C(/c1ccc(Br)cc1)c1ncccc1F] | ¹H NMR (300 MHz, DMSO-d₆): δ 8.53 8.51 (d, 1H), 7.88-7.82 (d, 1H), 7.72-7.65 (dd, 2H), 7.64-7.61 (dd, 1H), 7.48-7.45 (dd, 2H), 1.27-1.14 (m, 9H). |

### Intermediate 26a

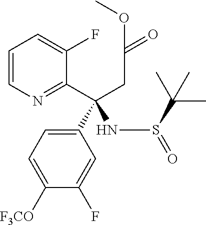 [SMILES: COC(=O)C[C@](N[S@](=O)C(C)(C)C)(c1ccc(OC(F)(F)F)c(F)c1)c1ncccc1F]

Step 4: (S)-methyl 3-((R)-1,1-dimethylethylsulflnamido)-3-(3-fluoro-4-(trifluoro methoxy)phenyl)-3-(3-fluoropyridin-2-yl)propanoate

To a solution of methyl acetate (9.7 mL, 12.4 mmol) in 2-MeTHF (336 mL) at −78° C. was added lithium diisopropylamide (1.8 M in heptane/THF/EtPh, 31 mL, 17.2 mmol) dropwise over 20-30 min. The solution was stirred at −78° C. for 30 min, followed by addition of Intermediate 25a (28 g, 6.89 mmol) in THF (84 mL) dropwise over a period of 20-30 min. The reaction mixture was further stirred at −78° C. for 10-15 min. After completion of reaction, the mixture was quenched with saturated aqueous NH₄Cl (84 mL) and slowly warmed to ambient temperature and diluted with water (84 mL). The resulting mixture was extracted with EtOAc (84 mL). The aqueous layer was back-extracted with EtOAc (56 mL). The combined organic layers were treated with brine (56 mL), dried over Na₂SO₄, and concentrated under reduced pressure. The thick yellow residue was triturated with n-pentane (84 mL) to afford a pale yellow precipitate. The precipitate was stirred for 5-10 min, filtered, and the solid was washed with n-pentane (56 mL) to afford the title compound as a pale yellow solid. MS (ESI pos. ion) m/z: 481.1 (MH+). ¹H NMR (300 MHz, CDCl₃) δ: 8.44-8.46 (m, 1H), 7.37-7.27 (m, 1H), 7.25-7.17 (m, 4H), 7.11-7.08 (m, 1H), 6.45 (s, 1H), 3.99-3.93 (d, J=17.1 Hz, 1H), 3.74-3.68 (d, J=17.4 Hz, 1H), 3.55 (s, 3H), 1.28 (s, 9H).

TABLE 10
Intermediates 26b-26d were prepared via anion addition analogous to the procedure described above

| 25b-25d | 26b-26d | Analytical data for 26b-d |
|---|---|---|
| 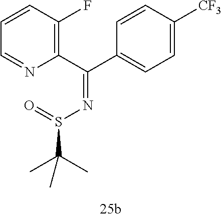 | 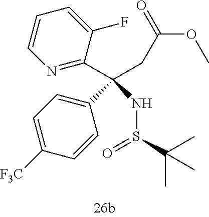 [SMILES: COC(=O)C[C@](N[S@](=O)C(C)(C)C)(c1ccc(C(F)(F)F)cc1)c1ncccc1F] | MS (ESI, positive ion) m/z: 447.5 (MH+). |
| 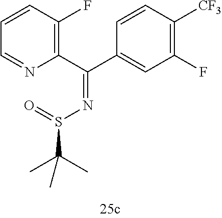 [SMILES: CC(C)(C)[S@@](=O)/N=C(/c1ccc(C(F)(F)F)c(F)c1)c1ncccc1F] | 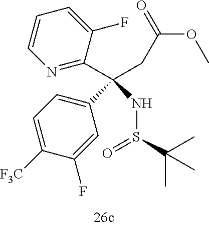 [SMILES: COC(=O)C[C@](N[S@](=O)C(C)(C)C)(c1ccc(C(F)(F)F)c(F)c1)c1ncccc1F] | ¹H NMR (300 MHz, DMSO-d₆): δ 8.55 (d, J = 4.2 Hz, 1H), 7.72-7.53 (m, 5H), 6.24 (s, 1H), 3.88-3.70 (m, 2H).3.46 (s, 3H), 1.19 (s, 9H). |
| 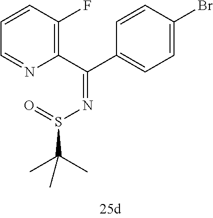 | 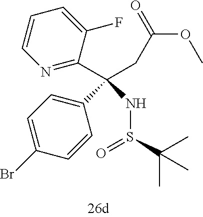 [SMILES: COC(=O)C[C@](N[S@](=O)C(C)(C)C)(c1ccc(Br)cc1)c1ncccc1F] | MS (ESI, positive ion) m/z: 459.2 (MH+). ¹H NMR (300 MHz, DMSO-d₆): δ 8.44-8.42 (m, 1H), 7.41-7.34 (m, 2H), 7.32-7.23 (m, 3H), 7.20-7.18 (m, 1H), 6.45 (s, 1H), 3.95-3.90 (d, 1H), 3.74-3.66 (d, 1H), 3.52 (s, 3H), 1.28 (s, 9H). |

### Intermediate 27a

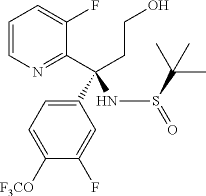 [SMILES: CC(C)(C)[S@@](=O)N[C@@](CCO)(c1ccc(OC(F)(F)F)c(F)c1)c1ncccc1F]

Step 5: (R)—N—((S)-1-(3-fluoro-4-(trifluoromethoxy)phenyl)-1-(3-fluoropyridin-2-yl)-3-hydroxypropyl)-2-methylpropane-2-sulfinamide

To a solution of (S)-methyl 3-((R)-1,1-dimethylethylsulfinamido)-3-(3-fluoro-4-(trifluoromethoxy)phenyl)-3-(3-fluoropyridin-2-yl)propanoate (23 g, 47.9 mmol) in THF (69 mL) at 0° C. was slowly added a solution of LiBH₄ (2.0 M in THF, 23 mL, 46 mmol) over a period of 20 min. The reaction mixture was allowed to warm to ambient temperature and was then stirred for 15 h at the same temperature. The reaction was quenched with saturated NH₄Cl (69 mL), warmed to room temperature, diluted with water, and extracted with EtOAc (69 mL). The aqueous phase was extracted with EtOAc (46 mL). The combined organic layers were washed with brine (46 mL), dried over Na₂SO₄ and concentrated under reduced pressure. The residue was triturated with n-pentane (69 mL), and the precipitate was filtered and washed with n-pentane (46 mL) to afford the title compound as an off white solid. MS (ESI pos. ion) m/z: 453.1 (MH+). ¹H NMR (300 MHz, CDCl₃) δ: 8.47-8.44 (m, 1H), 7.38-7.28 (m, 2H), 7.25-7.15 (m, 2H), 7.15-7.05 (m, 1H), 5.91 (s, 1H), 3.93-3.87 (m, 1H), 3.40-3.33 (m, 1H), 3.27-3.17 (m, 1H), 2.71-2.65 (m, 2H), 1.26 (s, 9H).

TABLE 11
Intermediates 27b-27d were prepared via ester reduction analogous to the procedure described above

| Intermediate | Intermediate | |
|---|---|---|
| 26b-26d | 27b-27d | Analytical data for 27b-d |
| 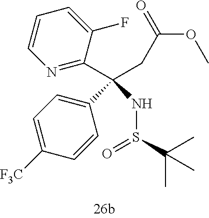 | 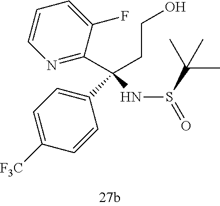 [SMILES: CC(C)(C)[S@@](=O)N[C@@](CCO)(c1ccc(C(F)(F)F)cc1)c1ncccc1F] | MS (ESI, positive ion) m/z: 419.5 (MH+). ¹H NMR (300 MHz, DMSO-d₆): δ 8.54-8.52 (d, J = 4.5 Hz, 1H), 7.69 (d, J = 8.1 Hz, 2H), 7.63-7.52 (m, 2H), 7.45 (d, J = 8.1 Hz, 2H), 6.41 (s, 1H), 4.77-4.74 (t, J = 4.2 Hz, 1H), 3.50-3.47 (m, 1H), 3.08-3.07 (m, 1H), 2.93-2.88 (m, 1H), 2.62-2.60 (m, 1H), 1.15 (s, 9H). |
| 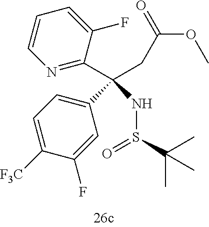 | 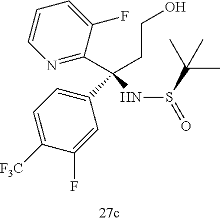 [SMILES: CC(C)(C)[S@@](=O)N[C@@](CCO)(c1ccc(C(F)(F)F)c(F)c1)c1ncccc1F] | MS (ESI, positive ion) m/z: 437.5 (MH+). ¹H NMR (300 MHz, DMSO-d₆): δ 8.53-8.52 (m, 1H), 7.72-7.52 (m, 4H), 7.45-7.41 (m, 1H), 6.53 (s, 1H), 6.40 (s, 1H), 4.78-4.72 (m, 1H), 3.49-3.45 (m, 1H), 2.95-2.87 (m, 1H), 2.63-2.56 (m, 1H), 1.18 (s, 9H). |
| 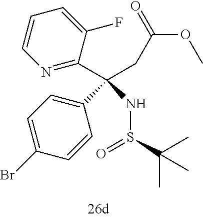 [SMILES: COC(=O)C[C@](N[S@](=O)C(C)(C)C)(c1ccc(Br)cc1)c1ncccc1F] | 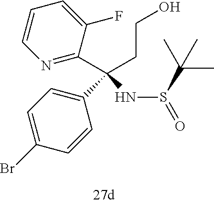 [SMILES: CC(C)(C)[S@@](=O)N[C@@](CCO)(c1ccc(Br)cc1)c1ncccc1F] | |

### Example 152

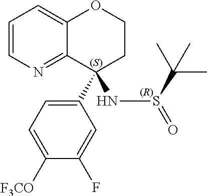

Step 6: (R)—N—((S)-4-(3-fluoro-4-(trifluoromethoxy)phenyl)-3,4-dihydro-2H-pyrano[3,2-b]pyridin-4-yl)-2-methylpropane-2-sulfinamide

To a solution of NaH (33 g, 530.9 mmol, Note: 60% in mineral oil material was washed with hexane and decanted, then with n-pentane (2×) and decanted and used in the reaction) in DMF (700 mL) was added portionwise (R)—N—((S)-1-(3-fluoro-4-(trifluoromethoxy)phenyl)-1-(3-fluoropyridin-2-yl)-3-hydroxypropyl)-2-methylpropane-2-sulfinamide (100 g, 88.4 mmol) at 0° C. over 30 min. The reaction mixture was stirred at 0° C. for 15 min and was then warmed to ambient temperature and stirred for 15 min. The mixture was then further heated at 65° C. for 15 h. The reaction was allowed to cool to room temperature, and was diluted with water (5 L) to give an off white precipitate. The precipitate was stirred at room temperature for 2-3 h. The precipitate was filtered and washed with water to afford the title compound as an off white solid. MS (ESI pos. ion) m/z: 433.5 (MH+). ¹H NMR (300 MHz, MeOH-d₄) δ: 8.19 (dd, J=3.9, 1.9 Hz, 1H), 7.24-7.50 (m, 4H), 7.09-7.23 (m, 1H), 4.46-4.59 (m, 1H), 4.14 (ddd, J=11.5, 8.1, 3.1 Hz, 1H), 2.74 (ddd, J=14.6, 7.3, 3.1 Hz, 1H), 2.61 (ddd, J=14.6, 7.9, 3.2 Hz, 1H), 1.23 (s, 9H).

TABLE 12
Intermediates 28b-28d were prepared via cyclization analogous to the procedure described above

| 27b-27d | 28b-28d | Analytical data for 28b-d |
|---|---|---|
| 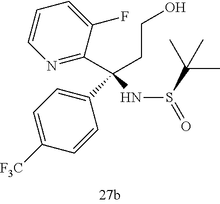 [SMILES: CC(C)(C)[S@@](=O)N[C@@](CCO)(c1ccc(C(F)(F)F)cc1)c1ncccc1F] | 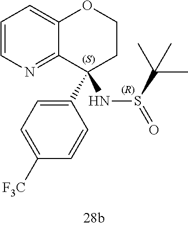 | ¹H NMR (300 MHz, DMSO-d₆): δ 8.13 (dd, J = 1.5, 4.2 Hz, 1H), 7.68 (d, J = 8.1 Hz, 2H), 7.54 (d, J = 8.4 Hz, 2H), 7.34- 7.24 (m, 2H), 4.64-4.54 (m, 1H), 4.21- 4.14 (m, 1H), 4.06-3.99 (m, 1H), 2.62- 2.58 (m, 1H), 1.18 (s, 9H). |
| 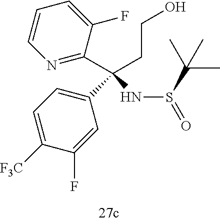 [SMILES: CC(C)(C)[S@@](=O)N[C@@](CCO)(c1ccc(C(F)(F)F)c(F)c1)c1ncccc1F] | 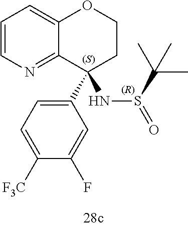 | ¹H NMR (300 MHz, CDCl₃): δ 8.24-8.22 (m, 1H), 7.56 (t, J = 7.8 Hz, 1H), 7.32- 7.18 (m, 4H), 5.31 (s, 1H), 4.38-4.32 (m, 1H), 4.07-3.99 (m, 1H), 2.79-2.61 (m, 1H), 1.19 (s, 9H). |
| 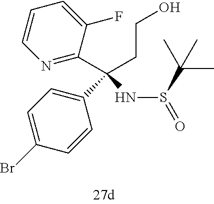 | 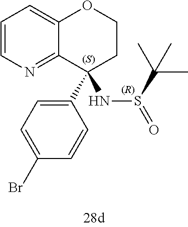 | MS (ESI, positive ion) m/z: 409.9 (MH+). ¹H NMR (300 MHz, DMSO-d₆): δ 8.22- 8.20 (m, 1H), 7.42-7.39 (dd, 2H), 7.25- 7.17 (m, 4H), 5.37-5.28 (dd, 2H), 4.34- 4.28 (m, 1H), 4.05-3.97 (m, 1H), 2.71- 2.63 (m, 2H), 1.18 (s, 9H) |

### Intermediate 17

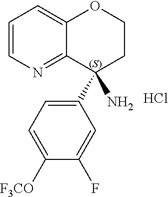

Step 7: (S)-4-(3-fluoro-4-(trifluoromethoxy)phenyl)-3,4-dihydro-2H-pyrano[3,2-b]pyridin-4-amine hydrochloride

To a 0° C. solution of ((R)—N—((S)-4-(3-fluoro-4-(trifluoromethoxy)phenyl)-3,4-dihydro-2H-pyrano[3,2-b]pyridin-4-yl)-2-methylpropane-2-sulfinamide (180 g, 41.6 mmol) in DCM (260 mL) was added 20% HCl in 1,4-dioxane (720 mL) dropwise. The reaction mixture was stirred for 4-5 h at room temperature. The reaction mixture was then concentrated and triturated with diethyl ether (1.8 L) and n-pentane (1.8L) respectively to afford an off white precipitate. The diethyl ether layer and pentane layer were decanted from the off white precipitate. The off white precipitate was dried under reduced pressure to afford the title compound as an off white solid. MS (APCI pos. ion) m/z: 329.1 (MH+). ¹H NMR (400 MHz, DMSO-d₆) δ: 9.43 (bs, 3H), 8.34-8.32 (m, 1H), 7.72-7.62 (m, 2H), 7.48-7.47 (m, 2H), 7.07-7.04 (d, J=8.4, 8.8 Hz, 1H), 4.38-4.33 (m, 1H), 3.82-3.79 (m, 1H), 2.80-2.77 (m, 1H), 2.69-2.65 (m, 1H).

TABLE 13
Intermediates 15 and 29c-29d were prepared via deprotection analogous to the procedure described above

| 28b-28d | 15, 29c-d | Analytical data for 15, 29c-d |
|---|---|---|
| 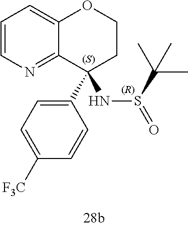 | 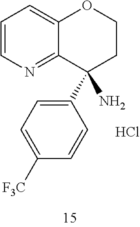 [SMILES: N[C@]1(c2ccc(C(F)(F)F)cc2)CCOc2cccnc21 ; Cl] | MS (ESI, positive ion) m/z: 295.1 (MH+). ¹H NMR (400 MHz, DMSO-d₆): δ 9.44 (bs, 3H), 8.32-8.31 (m, 1H), 7.82 (d, J = 8.4 Hz, 2H), 7.52-7.46 (m, 4H), 4.43-4.38 (m, 1H), 3.81-3.75 (m, 1H), 2.71-2.68 (m, 2H). |
| 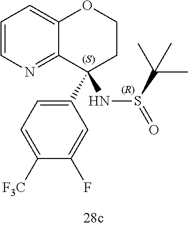 | 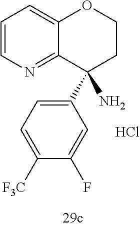 [SMILES: N[C@]1(c2ccc(C(F)(F)F)c(F)c2)CCOc2cccnc21 ; Cl] | MS (ESI, positive ion) m/z: 312.9 (MH+). ¹H NMR (400 MHz, DMSO-d₆): δ 9.52 (bs, 3H), 8.34-8.33 (m, 1H), 7.87 (t, J = 8 Hz, 1H), 7.73 (d, J = 12.4 Hz, 1H) 7.51-7.47 (m, 2H), 7.16 (d, J = 8.8 Hz, 1H), 4.44-4.41 (m, 1H), 3.92-3.82 (m, 2H), 2.81-2.66 (m, 2H). |
| 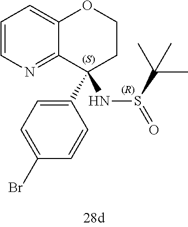 | 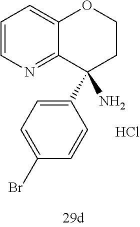 [SMILES: N[C@]1(c2ccc(Br)cc2)CCOc2cccnc21 ; Cl] | MS (ESI, positive ion) m/z: 305.6 (MH+). ¹H NMR (300 MHz, DMSO-d₆):): δ 9.34 (bs, 3H), 8.34-8.32 (m, 1H), 7.66-7.63 (m, 2H), 7.50-7.44 (m, 2H), 7.25-7.22 (dd, 2H), 4.42- 4.38 (m, 1H), 3.80-3.74 (m, 1H), 2.69-2.53 (m, 2H). |

### Intermediate 30

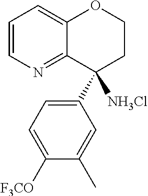

### (S)-4-(3-methyl-4-(trifluoromethoxy)phenyl)-3,4-dihydro-2H-pyrano[3,2-b]pyridin-4-amine hydrochloride

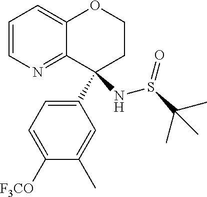 [SMILES: Cc1cc([C@@]2(N[S@@](=O)C(C)(C)C)CCOc3cccnc32)ccc1OC(F)(F)F]

Step 1: (S)-2-methyl-N-((S)-4-(3-methyl-4-(trifluoromethoxy)phenyl)-3,4-dihydro-2H-pyrano[3,2-b]pyridin-4-yl)propane-2-sulfinamide

This compound was prepared analogously to Intermediate 17 Step 2 using 4-bromo-2-methyl-1-(trifluoromethoxy)benzene. MS (ESI, positive ion) m/z: 429.0 (MH+).

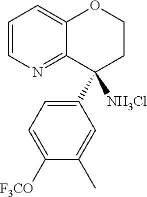

Step 2: (S)-4-(3-methyl-4-(trifluoromethoxy)phenyl)-3,4-dihydro-2H-pyrano[3,2-b]pyridin-4-amine hydrochloride

This compound was prepared analogously to Intermediate 17 Step 3 using (S)-2-methyl-N—((S)-4-(3-methyl-4-(trifluoromethoxy)phenyl)-3,4-dihydro-2H-pyrano[3,2-b]pyridin-4-yl)propane-2-sulfinamide. MS (ESI, positive ion) m/z: 308.0 (M-NH₂).

### Intermediate 31

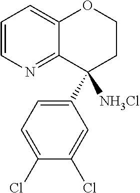

### (S)-4-(3,4-dichlorophenyl)-3,4-dihydro-2H-pyrano[3,2-b]pyridin-4-amine hydrochloride

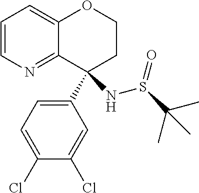 [SMILES: CC(C)(C)[S@](=O)N[C@]1(c2ccc(Cl)c(Cl)c2)CCOc2cccnc21]

Step 1: (S)—N—((S)-4-(3,4-dichlorophenyl)-3,4-dihydro-2H-pyrano[3,2-b]pyridin-4-yl)-2-methylpropane-2-sulfinamide

This compound was prepared analogously to Intermediate 17 Step 2 using 1-bromo-3,4-dichlorobenzene. MS (ESI, positive ion) m/z: 398.9, 400.9 (MH+).

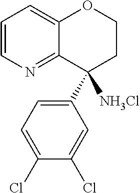

Step 2: (S)-4-(3,4-dichlorophenyl)-3,4-dihydro-2H-pyrano[3,2-b]pyridin-4-amine hydrochloride

This compound was prepared analogously to Intermediate 17 Step 3 using (S)—N—((S)-4-(3,4-dichlorophenyl)-3,4-dihydro-2H-pyrano[3,2-b]pyridin-4-yl)-2-methylpropane-2-sulfinamide. MS (ESI, positive ion) m/z: 295.0 (MH+).

### Intermediate 32

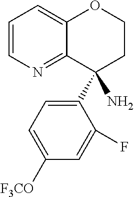 [SMILES: N[C@]1(c2ccc(OC(F)(F)F)cc2F)CCOc2cccnc21]

### (S)-4-(2-fluoro-4-(trifluoromethoxy)phenyl)-3,4-dihydro-2H-pyrano[3,2-b]pyridin-4-amine

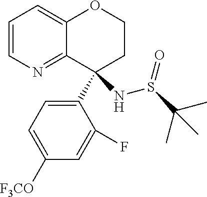 [SMILES: CC(C)(C)[S@](=O)N[C@]1(c2ccc(OC(F)(F)F)cc2F)CCOc2cccnc21]

Step 1: (S)—N—((S)-4-(2-fluoro-4-(trifluoromethoxy)phenyl)-3,4-dihydro-2H-pyrano[3,2-b]pyridin-4-yl)-2-methylpropane-2-sulfinamide

This compound was prepared analogously to Intermediate 17 Step 2 using 1-bromo-2-fluoro-4-(trifluoromethoxy)benzene. MS (ESI, positive ion) m/z: 433.0 (MH+).

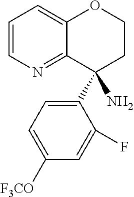 [SMILES: N[C@]1(c2ccc(OC(F)(F)F)cc2F)CCOc2cccnc21]

Step 2: (S)-4-(2-fluoro-4-(trifluoromethoxy)phenyl)-3,4-dihydro-2H-pyrano[3,2-b]pyridin-4-amine

This compound was prepared analogously to Intermediate 17 Step 3 using (S)—N—((S)-4-(2-fluoro-4-(trifluoromethoxy)-phenyl)-3,4-dihydro-2H-pyrano[3,2-b]pyridin-4-yl)-2-methylpropane-2-sulfinamide. MS (ESI, positive ion) m/z: 329.0 (MH+).

### Intermediate 33

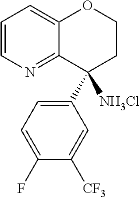

### (S)-4-(4-fluoro-3-(trifluoromethyl)phenyl)-3,4-dihydro-2H-pyrano[3,2-b]pyridin-4-amine hydrochloride

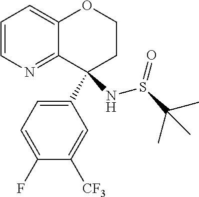 [SMILES: CC(C)(C)[S@](=O)N[C@]1(c2ccc(F)c(C(F)(F)F)c2)CCOc2cccnc21]

Step 1: (S)—N—((S)-4-(4-fluoro-3-(trifluoromethyl)phenyl)-3,4-dihydro-2H-pyrano[3,2-b]pyridin-4-yl)-2-methylpropane-2-sulfonamide

This compound was prepared analogously to Intermediate 17 Step 2 using 4-fluoro-3-(trifluoromethyl)bromobenzene. MS (ESI, positive ion) m/z: 416.9 (MH+).

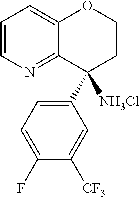

Step 2: (S)-4-(4-fluoro-3-(trifluoromethyl)phenyl)-3,4-dihydro-2H-pyrano[3,2-b]pyridin-4-amine hydrochloride

This compound was prepared analogously to Intermediate 17 Step 3 using (S)—N—((S)-4-(4-fluoro-3-(trifluoromethyl)phenyl)-3,4-dihydro-2H-pyrano[3,2-b]pyridin-4-yl)-2-methylpropane-2-sulfinamide. MS (ESI, positive ion) m/z: 313.1 (MH+).

### Intermediate 34

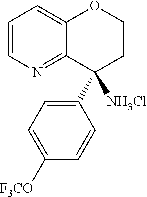

### (S)-4-(4-(trifluoromethoxy)phenyl)-3,4-dihydro-2H-pyrano[3,2-b]pyridin-4-amine hydrochloride

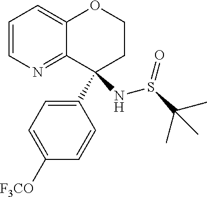 [SMILES: CC(C)(C)[S@](=O)N[C@]1(c2ccc(OC(F)(F)F)cc2)CCOc2cccnc21]

Step 1: (S)-2-methyl-N—((S)-4-(4-(trifluoromethoxy)phenyl)-3,4-dihydro-2H-pyrano[3,2-b]pyridin-4-yl)propane-2-sulfonamide

This compound was prepared analogously to Intermediate 17 Step 2 using 1-bromo-4-(trifluoromethoxy)-benzene. MS (ESI, positive ion) m/z: 415.0 (MH+).

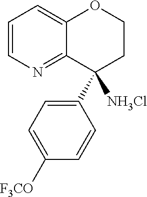

Step 2: (S)-4-(4-(trifluoromethoxy)phenyl)-3,4-dihydro-2H-pyrano[3,2-b]pyridin-4-amine hydrochloride

This compound was prepared analogously to Intermediate 17 Step 3 using (S)-2-methyl-N—((S)-4-(4-(trifluoromethoxy)-phenyl)-3,4-dihydro-2H-pyrano[3,2-b]pyridin-4-yl)propane-2-sulfinamide. MS (ESI, positive ion) m/z: 311.0 (MH+).

### Intermediate 34a

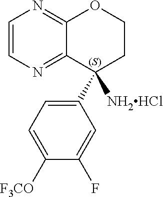

### (S)-8-(3-fluoro-4-(trifluoromethoxy)phenyl)-7,8-dihydro-6H-pyrano[2,3-b]pyrazin-8-amine hydrochloride

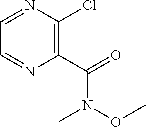

Step 1: 3-chloro-N-methoxy-N-methylpyrazine-2-carboxamide

To a 0° C. solution of 3-chloropyrazine-2-carboxylic acid (9 g, 56.9 mmol) in THF (90 mL) was added 1,1′-carbonyldiimidazole (10.15 g, 62.65 mmol). The reaction mixture was stirred for 3 h at room temperature. The mixture was then cooled to 0° C. and treated with TEA (23.77 mL, 170.88 mmol) followed by N,O -dimethylhydroxylamine hydrochloride (6.07 g, 62.57 mmol). The resulting reaction mixture was stirred for 12 h at room temperature. The reaction was then diluted with ice water (10 times) and extracted with EtOAc (3×100 mL). The combined EtOAc layers were concentrated in vacuo and purified by silica gel column chromatography, using 40% EtOAc in petroleum ether as the eluent to afford the title compound. MS (ESI pos. ion) m/z: 202.0 (MH+). ¹H NMR (400 MHz, CDCl₃) δ: 8.52 (d, J=2.4 Hz, 1H), 8.45 (d, J=2.8 Hz, 1H), 3.58 (s, 3H), 3.43 (s, 3H).

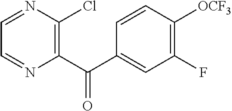 [SMILES: O=C(c1ccc(OC(F)(F)F)c(F)c1)c1nccnc1Cl]

Step 2: (3-chloropyrazin-2-yl) (3-fluoro-4-(trifluoromethoxy)phenyl)methanone

To a 0° C. solution of 4-bromo-2-fluoro-1-(trifluoromethoxy)benzene (19.9 g, 77.2 mmol) and THF (80 mL) was added a iPrMgCl solution (2M in Et₂O, 38.65 mL, 77.31 mmol) dropwise. The reaction mixture was stirred for 2 h at room temperature. The reaction was then cooled to −60° C. and a solution of 3-chloro-N-methoxy-N-methylpyrazine-2-carboxamide (7.77 g, 38.65 mmol) in THF (20 mL) was added dropwise. The reaction mixture was stirred for 2 h at ambient temperature. The reaction mixture was then quenched with 20% aqueous NH₄Cl and the reaction was extracted with EtOAc (3×50 mL). The combined organic layers were dried with Na₂SO₄ and concentrated in vacuo. The resulting residue was purified by silica gel column chromatography using 12% EtOAc in hexanes as the eluent to afford the title compound. MS (ESI pos. ion) m/z: 321.0 (MH+). ¹H NMR (400 MHz, CDCl₃) δ 8.62 (d, J=2.5 Hz, 1H), 8.60 (d, J=2.4 Hz, 1H), 7.79 (dd, J=2, 10 Hz, 1H), 7.66 (ddd, J=8.6, 2.1, 1.3 Hz, 1H), 7.45 (ddd, J=8.6, 7.2, 1.4 Hz, 1H).

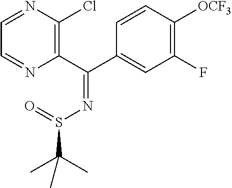 [SMILES: CC(C)(C)[S@@](=O)/N=C(/c1ccc(OC(F)(F)F)c(F)c1)c1nccnc1Cl]

Step 3: (R,Z)—N-((3-chloropyrazin-2-yl)(3-fluoro-4-(trifluoromethoxy)phenyl)methylene)-2-methylpropane-2-sulfinamide

To a solution of (3-chloropyrazin-2-yl) (3-fluoro-4-(trifluoromethoxy)phenyl)methanone (5.5 g, 17.18 mmol), THF (5.5 mL) and titanium (IV) ethoxide (7.8 g, 342 mmol) was added (R)-2-methylpropane-2-sulfinamide (2.28 g, 18.84 mmol) in one lot. The reaction mixture was stirred overnight at 75° C. The reaction mixture was diluted with EtOAc (80 mL) and brine (20 mL), filtered over Celite® brand filter agent, extracted with 10% MeOH in EtOAc. The organic layers were concentrated in vacuo. The residual liquid was purified by silica gel column chromatography with 15-85% EtOAc in petroleum ether as eluent to afford the title compound. MS (ESI pos. ion) m/z: 423.9 (MH+). ¹H NMR (400 MHz, CDCl₃) δ: 8.58 (s, 1H), 8.47 (dd, J=2.6, 0.6 Hz, 1H), 7.54 (dd, J=10.7, 2.2 Hz, 1H), 7.35 (td, J=7.9, 7.2, 1.4 Hz, 1H), 1.36 (s, 9H).

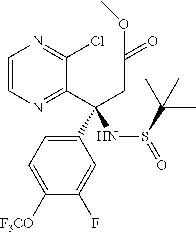 [SMILES: COC(=O)C[C@](N[S@](=O)C(C)(C)C)(c1ccc(OC(F)(F)F)c(F)c1)c1nccnc1Cl]

Step 4: (S)-methyl 3-(3-chloropyrazin-2-yl)-3-((R)-1,1-dimethylethylsulfinamido)-3-(3-fluoro-4-(trifluoromethoxy)phenyl)propanoate

To a −78° C. solution of methyl acetate (1.63 mL, 20 mmol) in 2-MeTHF (76.5 mL) was added lithium diisopropylamide (1.8M in heptane/THF/EtPh, 13.4 mL, 24.11 mmol) dropwise over 20-30 min. The solution was stirred at −78° C. for 30 min, followed by addition of a solution of (R,Z)—N-((3-chloropyrazin-2-yl)(3-fluoro-4-(trifluoromethoxy)phenyl)methylene)-2-methylpropane-2-sulfinamide (5.1 g, 12.05 mmol) in 2-MeTHF (15.3 mL) dropwise over 20-30 min. The reaction mixture was stirred at −78° C. for 10-15 min. The reaction was then quenched with 10% aqueous NH₄Cl solution and extracted with EtOAc (3×50 mL). The combined organic layers were washed with brine, dried over Na₂SO₄ and concentrated in vacuo. The residue was purified by column chromatography (neutral aluminum oxide) with 10-40% EtOAc in petroleum ether as the eluent to afford the title compound. MS (ESI pos. ion) m/z: 498.4 (MH+). ¹H NMR (400 MHz, CDCl₃) δ: 8.63 (dd, J=2.4, 0.5 Hz, 1H), 8.43 (dd, J=2.5, 0.6 Hz, 1H), 7.23 (tq, J=7.6, 1.2 Hz, 1H), 7.14 (dd, J=11.1, 2.3 Hz, 1H), 7.00-6.95 (m, 1H), 6.61 (s, 1H), 4.13 (d, J=16.8 Hz, 1H), 3.55 (d, J=16.8 Hz, 1H), 3.53 (s, 3H), 1.34 (s, 9H).

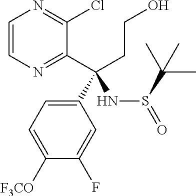 [SMILES: CC(C)(C)[S@@](=O)N[C@@](CCO)(c1ccc(OC(F)(F)F)c(F)c1)c1nccnc1Cl]

Step 5: (R)—N—((S)-1-(3-chloropyrazin-2-yl)-1-(3-fluoro-4-(trifluoromethoxy)phenyl)-3-hydroxypropyl)-2-methylpropane-2-sulfinamide

To a 0° C. solution of (S)-methyl 3-(3-chloropyrazin-2-yl)-3-((R)-1,1-dimethylethylsulfinamido)-3-(3-fluoro-4-(trifluoromethoxy)phenyl)propanoate (4.37 g, 8.79 mmol) in THF (13.11 mL) was added LiBH₄ (2.0M in THF, 4.4 mL) slowly over a period of 20 min. The reaction mixture was allowed to warm to room temperature and stirred overnight at the same temperature. The reaction was quenched with 10% aqueous NH₄Cl, diluted with water, and extracted with EtOAc (3×50 ml). The combined organic layers were washed with brine, dried over Na₂SO₄ and concentrated in vacuo. The residue thus obtained was purified by column chromatography (neutral aluminum oxide) with 70% EtOAc in petroleum ether as eluent to afford the title compound. MS (ESI pos. ion) m/z: 470.2 (MH+).

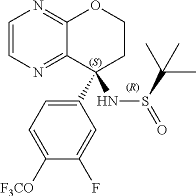

Step 6: (R)—N—((S)-8-(3-fluoro-4-(trifluoromethoxy)phenyl)-7,8-dihydro-6H-pyrano[2,3-b]pyrazin-8-yl)-2-methylpropane-2-sulfonamide

To a 0° C. solution of (R)—N—((S)-1-(3-chloropyrazin-2-yl)-1-(3-fluoro-4-(trifluoromethoxy)phenyl)-3-hydroxypropyl)-2-methylpropane-2-sulfinamide (1.3 g, 2.77 mmol) in DMF (13 mL) was added 60% NaH (0.66 g, 16.62 mmol) in portions over 30 min. The reaction mixture was stirred at 0° C. for 15 min and then warmed to ambient temperature. After stirring for a further 15 min., the reaction was heated to 65° C. for 7 h. The reaction was allowed to cool to room temperature and poured into ice water. The aqueous solution was extracted with EtOAc (3×20 mL). The combined EtOAc layers were concentrated to afford the title compound. ¹H NMR (400 MHz, CDCl₃) δ: 8.30 (d, J=2.5 Hz, 1H), 8.26 (d, J=2.4 Hz, 1H), 7.28-7.25 (m, 2H), 7.16 (dd, J=2.4, 1.2 Hz, 1H), 5.05 (s, 1H), 4.57 (d, J=12.4 Hz, 1H), 4.23 (td, J=11.5, 2.5 Hz, 1H), 2.89-2.83 (m, 1H), 2.73-2.69 (m, 1H), 1.22 (s, 9H).

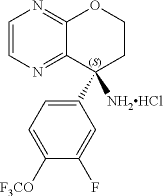

Step 7: (S)-8-(3-fluoro-4-(trifluoromethoxy)phenyl)-7,8-dihydro-6H-pyrano[2,3-b]pyrazin-8-amine hydrochloride. To a 0° C. solution of (R)—N—((S)-8-(3-fluoro-4-(trifluoromethoxy)phenyl)-7,8-dihydro-6H-pyrano[2,3-b]pyrazin-8-yl)-2-methylpropane-2-sulfinamide (0.5 g, 1.15 mmol) in DCM (2.5 mL) was added 4M HCl in 1,4-dioxane (5 mL, 20 mmol). The reaction mixture was stirred for 2 h at ambient temperature. The reaction mixture was then concentrated under reduced pressure and triturated with petroleum ether and diethyl ether to afford the title compound as a pale yellow solid. MS (ESI pos. ion) m/z: 313.0 (M-NH₂). ¹H NMR (400 MHz, DMSO-d₆) δ: 9.44 (s, 3H), 8.49-8.42 (m, 2H), 7.64-7.69 (m, 2H), 7.18 (d, J=10.4 Hz, 1H), 4.55 (m, 1H), 3.99 (m, 1H), 2.85-2.06 (m, 1H), 2.69-2.51 (m, 1H).

### Intermediate 34b

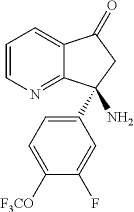 [SMILES: N[C@]1(c2ccc(OC(F)(F)F)c(F)c2)CC(=O)c2cccnc21]

### (S)-7-amino-7-(3-fluoro-4-(trifluoromethoxy)phenyl)-6,7-dihydro-5H-cyclopenta[b]pyridin-5-one

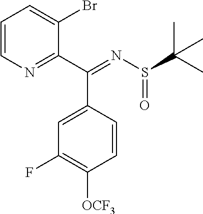 [SMILES: CC(C)(C)[S@@](=O)/N=C(\c1ccc(OC(F)(F)F)c(F)c1)c1ncccc1Br]

### (R)—N-((3-bromopyridin-2-yl)(3-fluoro-4-(trifluoromethoxy)-phenyl)methylene)-2-methylpropane-2-sulfinamide

To a RBF containing (3-bromopyridin-2-yl)(3-fluoro-4-(trifluoromethoxy)phenyl)methanone (25.10 g, 68.9 mmol) and (R)-2-methylpropane-2-sulfinamide (12.53 g, 103 mmol) under N₂ was added titanium (IV) ethoxide (52.4 g, 207 mmol). The reaction was flushed with N₂ (3×) and heated in a 70° C. oil bath and stirred under N₂ for 24 h. The reaction was rapidly poured into 400 mL of rapidly stirring brine. After 30 minutes, the resulting mixture was diluted with EtOAc (200 mL) and the mixture was stirred 10 min. The suspension was filtered through a Buchner funnel containing Celite® brand filter agent and the filter cake was washed with EtOAc (2×100 mL). The organic layer was separated, and the aqueous layer was washed with EtOAc (200 mL). The organic layers were combined, dried (MgSO₄), and concentrated to give the product as a yellow oily solid. Purification by silica gel column chromatography (0-50% EtOAc in hexanes) gave the title compound as a viscous yellow oil which slowly crystallized to a light yellow waxy solid. ¹H NMR (300 MHz, CDCl₃) δ: 8.64 (br. s., 1H), 7.94 (dd, J=8.2, 1.3 Hz, 1H), 7.51 (d, J=10.5 Hz, 1H), 7.25-7.36 (m, 4H), 1.36 (s, 9H).

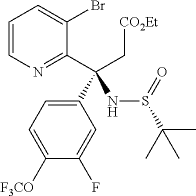 [SMILES: CCOC(=O)C[C@](N[S@](=O)C(C)(C)C)(c1ccc(OC(F)(F)F)c(F)c1)c1ncccc1Br]

Step 2: (S)-ethyl 3-(3-bromopyridin-2-yl)-3-((R)-1,1-dimethylethylsulfinamido)-3-(3-fluoro-4-(trifluoromethoxy)phenyl)propanoate

A solution of EtOAc (6.27 mL, 64.2 mmol) in 2-MeTHF (100 mL) in an oven-dried 3-necked RBF equipped with an addition funnel was cooled to −78° C. under nitrogen. The solution was treated with lithium diisopropylamide (2M in heptane/THF/ethylbenzene, 32.1 mL, 64.2 mmol) dropwise over 12 min. After 30 minutes, a solution of (R,E)-N-((3-bromopyridin-2-yl)(3-fluoro-4-(trifluoromethoxy)-phenyl)methylene)-2-methylpropane-2-sulfinamide (15 g, 32.1 mmol) in 2-MeTHF (65 ml) was added dropwise over 35 min via addition funnel. After 15 min, the reaction was quenched with saturated aqueous NH₄Cl (10 mL) and warmed to room temperature. The mixture was diluted with EtOAc (500 mL) and washed with saturated aqueous NH₄Cl (150 mL), water (150 mL) and brine (150 mL), dried over MgSO₄, concentrated in vacuo and purified by silica gel chromatography (eluent: 5-30% acetone/hexanes) to afford the title compound as a white solid. MS (ESI pos. ion) m/z: 555.0 (MH+). ¹H NMR (400 MHz, CDCl₃) δ: 8.65 (dd, J=1.5, 4.6 Hz, 1H), 7.83 (dd, J=1.5, 7.9 Hz, 1H), 7.15-7.24 (m, 2H), 7.10 (dd, J=1.9, 11.3 Hz, 1H), 6.96 (d, J=8.4 Hz, 1H), 6.77 (s, 1H), 4.15 (d, J=16.2 Hz, 1H), 3.95 (q, J=7.1 Hz, 2H), 3.49 (d, J=16.2 Hz, 1H), 1.25-1.41 (m, 9H), 1.04 (t, J=7.1 Hz, 3H).

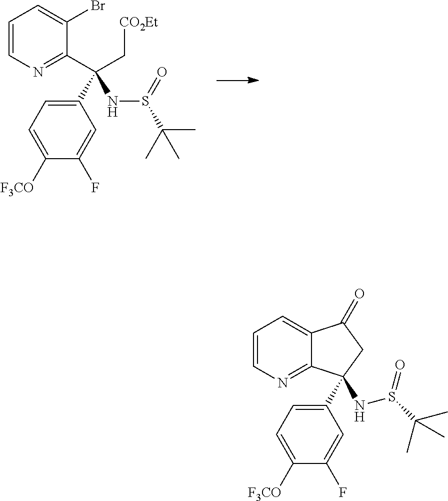 [SMILES: CCOC(=O)C[C@](N[S@](=O)C(C)(C)C)(c1ccc(OC(F)(F)F)c(F)c1)c1ncccc1Br ; CC(C)(C)[S@@](=O)N[C@]1(c2ccc(OC(F)(F)F)c(F)c2)CC(=O)c2cccnc21]

Step 3: (R)—N—((S)-7-(3-fluoro-4-(trifluoromethoxy)phenyl)-5-oxo-6,7-dihydro-5H-cyclopenta[b]pyridin-7-yl)-2-methylpropane-2-sulfinamide

A solution of (S)-ethyl 3-(3-bromopyridin-2-yl)-3-((R)-1,1-dimethylethylsulfinamido)-3-(3-fluoro-4-(trifluoromethoxy)-phenyl)propanoate (250 mg, 0.450 mmol) in THF (10 mL) in an oven-dried RBF was cooled to −80° C. (hexane/liquid nitrogen bath) under nitrogen. 1.7 M t-BuLi in pentane (0.794 mL, 1.350 mmol) was added dropwise. After the addition was complete, the reaction was stirred for 20 min, then quenched with iPrOH (2 mL), warmed to room temperature, diluted with EtOAc (50 mL) and washed with saturated aqueous NH₄Cl (50 mL), brine (50 mL), dried over MgSO₄, and concentrated in vacuo. The residue was purified by silica gel chromatography (eluent: 5-30% acetone in hexane) affording the title compound as a white solid. MS (ESI pos. ion) m/z: 430.9 (MH+). ¹H NMR (400 MHz, CDCl₃) δ: 8.88 (dd, J=1.6, 4.7 Hz, 1H), 8.11 (dd, J=1.6, 7.8 Hz, 1H), 7.37-7.61 (m, 2H), 7.28 (s, 2H), 4.53 (s, 1H), 3.68 (d, J=19.0 Hz, 1H), 3.43 (d, J=19.0 Hz, 1H), 1.13-1.29 (m, 9H).

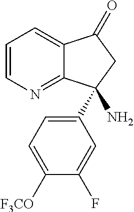 [SMILES: N[C@]1(c2ccc(OC(F)(F)F)c(F)c2)CC(=O)c2cccnc21]

Step 4: (S)-7-amino-7-(3-fluoro-4-(trifluoromethoxy)phenyl)-6,7-dihydro-5H-cyclopenta[b]pyridin-5-one

A solution of (R)—N—((S)-7-(3-fluoro-4-(trifluoromethoxy)phenyl)-5-oxo-6,7-dihydro-5H-cyclopenta[b]pyridin-7-yl)-2-methylpropane-2-sulfinamide (146 mg, 0.339 mmol) in EtOH (3 mL) and DCM (3 mL) was treated with 4M HCl in 1,4-dioxane (0.424 mL, 1.696 mmol). The reaction was stirred at room temperature. After 45 minutes, the reaction was concentrated in vacuo. The residue was dissolved in DCM (50 mL) and washed with saturated aqueous NaHCO₃ (2×25 mL), dried over MgSO₄ and concentrated in vacuo to afford the title compound as a yellow oil. MS (ESI pos. ion) m/z: 310.0 (M-NH₂). ¹H NMR (400 MHz, DMSO-d₆) δ: 8.84 (dd, J=1.6, 4.7 Hz, 1H), 8.11 (dd, J=1.7, 7.7 Hz, 1H), 7.51-7.64 (m, 2H), 7.39-7.50 (m, 1H), 7.16 (td, J=1.0, 8.6 Hz, 1H), 3.03-3.15 (m, 1H), 2.91-3.02 (m, 1H), 2.82 (br. s

### Intermediate 35

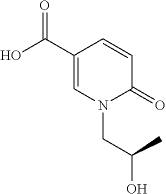

### (R)-1-(2-hydroxypropyl)-6-oxo-1,6-dihydropyridine-3-carboxylic acid

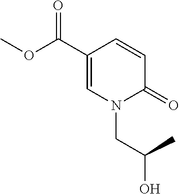

Step 1: (R)-methyl 1-(2-hydroxypropyl)-6-oxo-1,6-dihydropyridine-3-carboxylate

To a solution of (R)-1-aminopropan-2-ol (475 mg, 6.32 mmol) and MeOH (3 mL) was added methyl 2-oxo-2H-pyran-5-carboxylate (764 mg, 4.96 mmol). The reaction was stirred at room temperature. After 5 d, the reaction was concentrated in vacuo and adsorbed onto a plug of silica gel and chromatographed through a Redi-Sep® pre-packed silica gel column (12 g), eluting with 0-100% EtOAc in hexane, to provide the title compound as a light yellow solid. MS (ESI, positive ion) m/z: 212.0 (MH+).

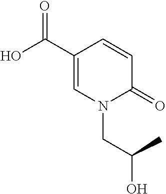

Step 2: (R)-1-(2-hydroxypropyl)-6-oxo-1,6-dihydropyridine-3-carboxylic acid

To a solution of (R)-methyl 1-(2-hydroxypropyl)-6-oxo-1,6-dihydropyridine-3-carboxylate (440 mg, 2.083 mmol) and THF (10 mL):MeOH (3 mL), was added LiOH (3 mL, 3.00 mmol). The reaction was stirred at room temperature. After 3 h, the reaction was concentrated in vacuo and then the aqueous solution was neutralized with 1N HCl and extracted with EtOAc (5×10 mL). The combined EtOAc layers were concentrated in vacuo to give the title compound as a light yellow solid. MS (ESI, positive ion) m/z: 198.1 (MH+).

### Intermediate 36

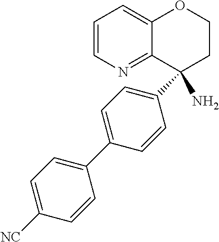 [SMILES: N#Cc1ccc(-c2ccc([C@@]3(N)CCOc4cccnc43)cc2)cc1]

### (S)-4′-(4-amino-3,4-dihydro-2H-pyrano[3,2-b]pyridin-4-yl)-[1,1′-biphenyl]-4-carbonitrile

To a solution of (S)-4-(4-bromophenyl)-3,4-dihydro-2H-pyrano[3,2-b]pyridin-4-amine hydrochloride (366 mg, 1.071 mmol) and dioxane (10 mL):water (0.5 mL), were added K₃PO₄ (682 mg, 3.21 mmol), (4-cyanophenyl)boronic acid (213 mg, 1.450 mmol), and A-Phos (76 mg, 0.107 mmol). The solution was stirred at 80° C. After 16 h, LC-MS showed only partial conversion. The reaction was treated with additional catalyst (25 mg) and (4-cyanophenyl)boronic acid (100 mg). After a further 24 h, the reaction temperature was raised to 100° C. After stirring for a further 72 h, LC-MS showed the starting material had been consumed. The reaction was allowed to cool to room temperature and filtered through a pad of Celite® brand filter agent. The Celite® brand filter agent was rinsed with EtOAc, and the filtrate was concentrated in vacuo. The residue was adsorbed onto a plug of silica gel and chromatographed through a Redi-Sep® pre-packed silica gel column (12 g), eluting with 0-100% EtOAc in hexanes, to provide the title compound as a golden foam. MS (ESI, positive ion) m/z: 328.1 (MH+). ¹H NMR (300 MHz, MeOH-d₄) δ: 8.17 (dd, J=4.2, 1.8 Hz, 1H), 7.72-7.89 (m, 4H), 7.59-7.70 (m, 2H), 7.21-7.40 (m, 4H), 4.24-4.39 (m, 1H), 3.94-4.08 (m, 1H), 2.32-2.53 (m, 2H).

### Example 153

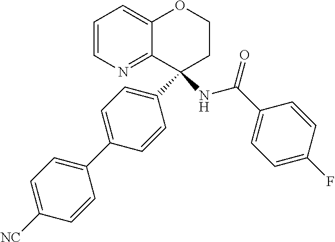 [SMILES: N#Cc1ccc(-c2ccc([C@@]3(NC(=O)c4ccc(F)cc4)CCOc4cccnc43)cc2)cc1]

### (S)—N-(4-(4′-cyano-[1,1′-biphenyl]-4-yl)-3,4-dihydro-2H-pyrano[3,2-b]pyridin-4-yl)-4-fluorobenzamide

To a solution of (S)-4′-(4-amino-3,4-dihydro-2H-pyrano[3,2-b]pyridin-4-yl)-[1,1′-biphenyl]-4-carbonitrile (78 mg, 0.238 mmol) and DCM (3 mL) was added DIPEA (0.100 mL, 0.574 mmol) and 4-fluorobenzoyl chloride (30 μL, 0.250 mmol). The solution was stirred at room temperature. After 2 h, the reaction was adsorbed onto a plug of silica gel and chromatographed through a Redi-Sep® pre-packed silica gel column (12 g), eluting with 0-25% EtOAc in hexanes to provide the title compound as a white foam. MS (ESI, positive ion) m/z: 450.0 (MH+). ¹H NMR (300 MHz, MeOH-d₄) δ: 8.18 (dd, J=4.0, 2.0 Hz, 1H), 7.75-7.91 (m, 6H), 7.63-7.74 (m, 2H), 7.43-7.53 (m, 2H), 7.25-7.43 (m, 2H), 7.09-7.24 (m, 2H), 4.32-4.46 (m, 1H), 3.95-4.16 (m, 1H), 3.34-3.46 (m, 1H), 2.87-3.04 (m, 1H).

### Example 154

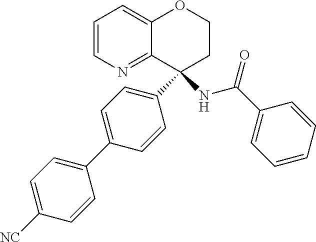 [SMILES: N#Cc1ccc(-c2ccc([C@@]3(NC(=O)c4ccccc4)CCOc4cccnc43)cc2)cc1]

### (S)—N-(4-(4′-cyano-[1,1′-biphenyl]-4-yl)-3,4-dihydro-2H-pyrano[3,2-b]pyridin-4-yl)benzamide

To a solution of (S)-4′-(4-amino-3,4-dihydro-2H-pyrano[3,2-b]pyridin-4-yl)[1,1′-biphenyl]-4-carbonitrile (69.7 mg, 0.213 mmol) DCM (3 mL) were added DIPEA (0.09 mL, 0.517 mmol) and benzoyl chloride (28 μL, 0.241 mmol). The solution was stirred at room temperature. After 3 h, the reaction was adsorbed onto a plug of silica gel and chromatographed through a Redi-Sep® pre-packed silica gel column (12 g), eluting with 0-25% EtOAc in hexane to provide the title compound as a white foam. MS (ESI, positive ion) m/z: 432.0 (MH+). ¹H NMR (300 MHz, MeOH-d₄) δ: 8.20 (dd, J=3.9, 2.0 Hz, 1H), 7.74-7.87 (m, 6H), 7.64-7.73 (m, 2H), 7.40-7.58 (m, 5H), 7.27-7.38 (m, 2H), 4.40 (dt, J=11.7, 4.0 Hz, 1H), 3.98-4.12 (m, 1H), 3.34-3.41 (m, 1H), 2.99-3.11 (m, 1H).

### Example 155

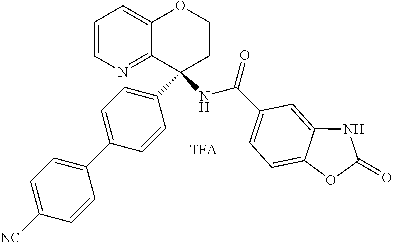 [SMILES: N#Cc1ccc(-c2ccc([C@@]3(NC(=O)c4ccc5oc(=O)[nH]c5c4)CCOc4cccnc43)cc2)cc1]

### (S)—N-(4-(4′-cyano-[1,1′-biphenyl]-4-yl)-3,4-dihydro-2H-pyrano[3,2-b]pyridin -4-yl)-2-oxo-2,3-dihydrobenzo[d]oxazole-5-carboxamide 2,2,2-trifluoroacetate

To a solution of (S)-4′-(4-amino-3,4-dihydro-2H-pyrano[3,2-b]pyridin-4-yl)-[1,1′-biphenyl]-4-carbonitrile (31 mg, 0.095 mmol) and DCM (3 mL) were added 2-oxo-2,3-dihydrobenzo[d]oxazole-5-carboxylic acid (25 mg, 0.140 mmol), DIPEA (0.05 mL, 0.287 mmol), and HATU (53 mg, 0.139 mmol). The reaction was stirred at room temperature. After 18 h, the residual reaction product was adsorbed onto a plug of silica gel and chromatographed through a Redi-Sep® pre-packed silica gel column (12 g), eluting with 0-50% EtOAc in hexanes, to provide fractions with overlapping spots. The fractions were concentrated in vacuo and purified by reverse-phase preparative HPLC [10-80% MeCN (0.1% TFA) in water (0.1% TFA)] to give the title compound as a white solid after lyophilization. MS (ESI, positive ion) m/z: 489.0 (MH+). ¹H NMR (300 MHz, DMSO-d₆) δ: 11.80 (s, 1H), 8.82 (s, 1H), 8.17 (dd, J=4.4, 1.6 Hz, 1H), 7.81-8.01 (m, 4H), 7.71 (d, J=8.5 Hz, 2H), 7.61 (dd, J=8.3, 1.8 Hz, 1H), 7.53 (d, J=1.6 Hz, 1H), 7.45 (d, J=8.5 Hz, 2H), 7.27-7.41 (m, 3H), 4.31-4.48 (m, 1H), 4.10 (t, J=9.0 Hz, 1H), 3.19-3.40 (m, 1H), 2.78-2.97 (m, 1H).

### Example 156

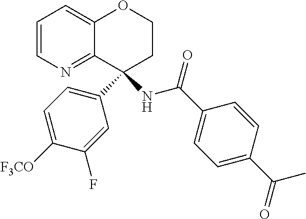 [SMILES: CC(=O)c1ccc(C(=O)N[C@]2(c3ccc(OC(F)(F)F)c(F)c3)CCOc3cccnc32)cc1]

### (S)-4-acetyl-N-(4-(3-fluoro-4-(trifluoromethoxy)phenyl)-3,4-dihydro-2H-pyrano[3,2-b]pyridin-4-yl)benzamide

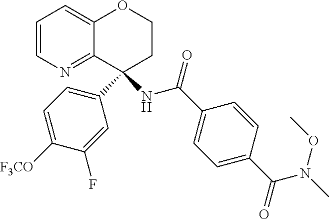 [SMILES: CON(C)C(=O)c1ccc(C(=O)N[C@]2(c3ccc(OC(F)(F)F)c(F)c3)CCOc3cccnc32)cc1]

Step 1: (S)—N1-(4-(3-fluoro-4-(trifluoromethoxy)phenyl)-3,4-dihydro-2H-pyrano[3,2-b]pyridin-4-yl)-N4-methoxy-N4-methylterephthalamide

To a RBF containing (S)-4-((4-(3-fluoro-4-(trifluoromethoxy)phenyl)-3,4-dihydro-2H-pyrano[3,2-b]pyridin-4-yl)carbamoyl)benzoic acid (682 mg, 1.432 mmol) and DCM (10 mL), were added N,O-dimethylhydroxylamine hydrochloride (140 mg, 1.432 mmol), DIPEA (0.748 mL, 4.29 mmol), and 50% T3P in EtOAc (3.01 mL, 3.58 mmol). The reaction was stirred at room temperature. After 1 h, the reaction was washed with water (2×15 mL). The aqueous layer was back extracted with DCM (2×10 mL). The combined DCM layers were dried over MgSO₄ and concentrated in vacuo to afford the title compound as a white solid. MS (ESI pos. ion) m/z: 520.1 (MH+). ¹H NMR (400 MHz, MeOH-d₄) δ: 8.19 (dd, J=4.1, 2.0 Hz, 1H), 7.86 (d, J=8.6 Hz, 2H), 7.69 (d, J=8.4 Hz, 2H), 7.28-7.47 (m, 4H), 7.23 (d, J=8.8 Hz, 1H), 4.35-4.45 (m, 1H), 4.03-4.15 (m, 1H), 3.56 (s, 3H), 3.33-3.39 (m, 4H), 2.98-3.08 (m, 1H).

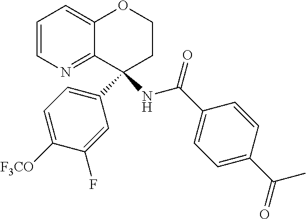 [SMILES: CC(=O)c1ccc(C(=O)N[C@]2(c3ccc(OC(F)(F)F)c(F)c3)CCOc3cccnc32)cc1]

Step 2: (S)-4-acetyl-N-(4-(3-fluoro-4-(trifluoromethoxy)phenyl)-3,4-dihydro-2H-pyrano[3,2-b]pyridin-4-yl)benzamide

To a 0° C. solution of (S)—N1-(4-(3-fluoro-4-(trifluoromethoxy)phenyl)-3,4-dihydro-2H-pyrano[3,2-b]pyridin-4-yl)-N4-methoxy-N4-methylterephthalamide (200 mg, 0.385 mmol) and THF (5 mL) was added methylmagnesium bromide (3M in Et₂O, 0.25 mL, 0.750 mmol). After 2 h, LC-MS showed ˜60% conversion to desired product (m/z=475 [MH+]). Another 0.25 mL of methylmagnesium bromide was added. After a further 1.5 h, the reaction was quenched with water (10 mL). The reaction was diluted with more water (20 mL) and EtOAc (30 mL). The aqueous layer was extracted with EtOAc (2×20 mL). The combined EtOAc layers were concentrated in vacuo and adsorbed onto a plug of silica gel and chromatographed through a Redi-Sep® pre-packed silica gel column (12 g), eluting with 0-50% EtOAc in hexanes, to provide the title compound as a white solid. MS (ESI pos. ion) m/z: 475.1 (MH+). ¹H NMR (300 MHz, MeOH-d₄) δ: 8.20 (dd, J=3.8, 2.2 Hz, 1H), 8.01-8.09 (m, 2H), 7.85-7.95 (m, 2H), 7.27-7.50 (m, 4H), 7.17-7.27 (m, 1H), 4.35-4.47 (m, 1H), 4.04-4.14 (m, 1H), 3.25-3.36 (m, 1H), 2.99-3.13 (m, 1H), 2.62 (s, 3H).

### Example 157

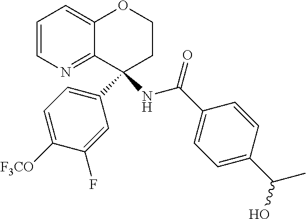 [SMILES: CC(O)c1ccc(C(=O)N[C@]2(c3ccc(OC(F)(F)F)c(F)c3)CCOc3cccnc32)cc1]

### N—((S)-4-(3-fluoro-4-(trifluoromethoxy)phenyl)-3,4-dihydro-2H-pyrano[3,2-b]pyridin-4-yl)-4-((R)-1-hydroxyethyl)benzamide and N—((S)-4-(3-fluoro-4-(trifluoromethoxy)phenyl)-3,4-dihydro-2H-pyrano[3,2-b]pyridin-4-yl)-4-((S)-1-hydroxyethyl)benzamide (1:1)

To a solution of (S)-4-acetyl-N-(4-(3-fluoro-4-(trifluoromethoxy)phenyl)-3,4-dihydro-2H-pyrano[3,2-b]pyridin-4-yl)benzamide (89 mg, 0.188 mmol) and MeOH (5 mL), was added NaBH₄ (8.52 mg, 0.225 mmol). After 2 h, the reaction was quenched with water and concentrated in vacuo. The residue was diluted with water (25 mL) and EtOAc (15 mL). The aqueous layer was extracted with EtOAc (10 mL). The combined EtOAc layers were dried over MgSO₄ and concentrated in vacuo to give the title compounds as a white solid. MS (ESI pos. ion) m/z: 477.0 (MH+). ¹H NMR (300 MHz, DMSO-d₆) δ: 8.79 (s, 1H), 8.13 (dd, J=4.1, 1.9 Hz, 1H), 7.77 (d, J=8.3 Hz, 2H), 7.44-7.59 (m, 2H), 7.39 (d, J=8.2 Hz, 2H), 7.16-7.34 (m, 3H), 5.25 (d, J=4.4 Hz, 1H), 4.76 (dd, J=6.4, 4.5 Hz, 1H), 4.34 (ddd, J=11.3, 7.9, 2.9 Hz, 1H), 4.08-4.21 (m, 1H), 3.17-3.28 (m, 1H), 2.91 (ddd, J=14.2, 7.7, 2.7 Hz, 1H), 1.31 (d, J=6.4 Hz, 3H).

### Example 158

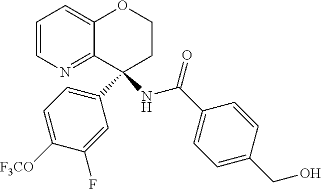 [SMILES: O=C(N[C@]1(c2ccc(OC(F)(F)F)c(F)c2)CCOc2cccnc21)c1ccc(CO)cc1]

### (S)—N-(4-(3-fluoro-4-(trifluoromethoxy)phenyl)-3,4-dihydro-2H-pyrano[3,2-b]pyridin-4-yl)-4-(hydroxymethyl)benzamide

To a 0° C. solution of (S)-4-((4-(3-fluoro-4-(trifluoromethoxy)phenyl)-3,4-dihydro-2H-pyrano[3,2-b]pyridin-4-yl)carbamoyl)benzoic acid (402 mg, 0.844 mmol) and THF (10 mL), was added 2M LiAlH₄ in THF (0.422 mL, 0.844 mmol) dropwise over 1 min. The reaction was allowed to warm to room temperature as the cooling bath expired. After 16 h, LC-MS showed only ˜1/3 conversion. Another 0.6 mL of LiAlH₄ was added dropwise at room temperature. After a further 16 h, the reaction was quenched with saturated aqueous potassium sodium tartrate. The aqueous solution was extracted with EtOAc (3×20 mL). The combined EtOAc layers were concentrated in vacuo and adsorbed onto a plug of silica gel and chromatographed through a GraceResolv® silica gel column (24 g), eluting with 0-50% EtOAc in hexanes, to provide the title compound as a white foam. MS (ESI pos. ion) m/z: 463.0 (MH+). ¹H NMR (300 MHz, DMSO-d₆) δ: 8.80 (s, 1H), 8.13 (dd, J=4.0, 2.0 Hz, 1H), 7.79 (d, J=8.3 Hz, 2H), 7.41-7.59 (m, 2H), 7.37 (d, J=8.3 Hz, 2H), 7.17-7.32 (m, 3H), 5.28 (t, J=5.8 Hz, 1H), 4.54 (d, J=5.7 Hz, 2H), 4.26-4.41 (m, 1H), 4.09-4.20 (m, 1H), 3.20-3.27 (m, 1H), 2.83-2.98 (m, 1H).

### Example 159

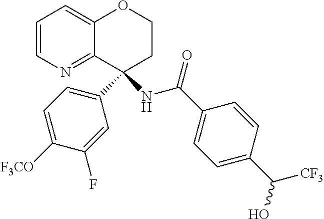 [SMILES: O=C(N[C@]1(c2ccc(OC(F)(F)F)c(F)c2)CCOc2cccnc21)c1ccc(C(O)C(F)(F)F)cc1]

### N—((S)-4-(3-fluoro-4-(trifluoromethoxy)phenyl)-3,4-dihydro-2H-pyrano[3,2-b]pyridin-4-yl)-4-((R)-2,2,2-trifluoro-1-hydroxyethyl)benzamide and N—((S)-4-(3-fluoro-4-(trifluoromethoxy)phenyl)-3,4-dihydro-2H-pyrano[3,2-b]pyridin-4-yl)-4-((S)-2,2,2-trifluoro-1-hydroxyethyl)benzamide (1:1)

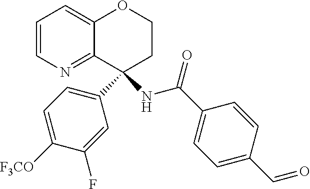 [SMILES: O=Cc1ccc(C(=O)N[C@]2(c3ccc(OC(F)(F)F)c(F)c3)CCOc3cccnc32)cc1]

Step 1: (S)—N-(4-(3-fluoro-4-(trifluoromethoxy)phenyl)-3,4-dihydro-2H-pyrano[3,2-b]pyridin-4-yl)-4-formylbenzamide

To a solution of (S)—N-(4-(3-fluoro-4-(trifluoromethoxy)phenyl)-3,4-dihydro-2H-pyrano[3,2-b]pyridin-4-yl)-4-(hydroxymethyl)benzamide (207 mg, 0.448 mmol) and DCM (10 mL), was added Dess-Martin periodinane (262 mg, 0.618 mmol). After 3 h, the reaction was treated with saturated aqueous Na₂S₂O₃ (5 mL). After stirring for 1 h, the organic layer was separated and the aqueous layer extracted with DCM (5 mL). The combined organic layers were concentrated in vacuo to give the title compound as a white solid. MS (ESI pos. ion) m/z: 461.0 (MH+). ¹H NMR (300 MHz, DMSO-d₆) δ: 10.08 (s, 1H), 9.11 (s, 1H), 8.13 (dd, J=4.1, 1.8 Hz, 1H), 7.89-8.05 (m, 4H), 7.42-7.61 (m, 2H), 7.16-7.38 (m, 3H), 4.25-4.42 (m, 1H), 4.06-4.19 (m, 1H), 3.24-3.36 (m, 1H), 2.78-2.94 (m, 1H).

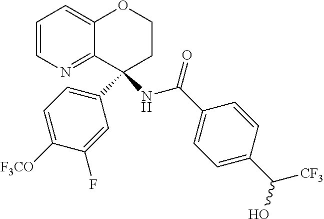 [SMILES: O=C(N[C@]1(c2ccc(OC(F)(F)F)c(F)c2)CCOc2cccnc21)c1ccc(C(O)C(F)(F)F)cc1]

Step 2: N—((S)-4-(3-fluoro-4-(trifluoromethoxy)phenyl)-3,4-dihydro-2H-pyrano[3,2-b]pyridin-4-yl)-4-((R)-2,2,2-trifluoro-1-hydroxyethyl)benzamide and N—((S)-4-(3-fluoro-4-(trifluoromethoxy)phenyl)-3,4-dihydro-2H-pyrano[3,2-b]pyridin-4-yl)-4-((S)-2,2,2-trifluoro-1-hydroxyethyl)benzamide (1:1)

To a −78° C. solution of (S)—N-(4-(3-fluoro-4-(trifluoromethoxy)phenyl)-3,4-dihydro-2H-pyrano[3,2-b]pyridin-4-yl)-4-formylbenzamide (206 mg, 0.447 mmol) and THF (5 mL), was added trimethyl(trifluoromethyl)silane (0.71 mL, 4.47 mmol) dropwise. After stirring for 15 min, 1M TBAF in THF (0.45 mL, 0.450 mmol) was added dropwise. After 1.5 h, the reaction was quenched with water and the aqueous layer was extracted with EtOAc (10 mL). The combined organic layers were concentrated in vacuo and adsorbed onto a plug of silica gel and chromatographed through a Redi-Sep® pre-packed silica gel column (12 g), eluting with hexanes (50 mL), 10% EtOAc in hexanes (50 mL), and then 50% EtOAc in hexanes (100 mL). The desired fractions were concentrated in vacuo. The material was suspended in hexanes and the hexanes decanted off. The gummy solid was taken up in Et₂O and concentrated in vacuo to give the title compound as a white foam. MS (ESI pos. ion) m/z: 530.9 (MH+). ¹H NMR (300 MHz, CDCl₃) δ: 8.51 (s, 1H), 8.30 (dd, J=4.1, 1.9 Hz, 1H), 7.79 (dd, J=8.4, 1.8 Hz, 2H), 7.51 (d, J=6.9 Hz, 2H), 7.27-7.40 (m, 3H), 7.17-7.25 (m, 2H), 4.94-5.13 (m, 1H), 4.30-4.51 (m, 1H), 3.94-4.15 (m, 1H), 3.67-3.89 (m, 1H), 3.02-3.10 (m, 1H), 2.70-2.91 (m, 1H).

### Example 160

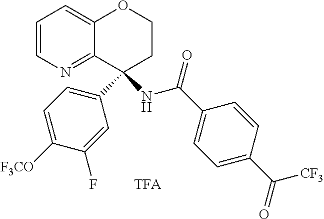 [SMILES: O=C(N[C@]1(c2ccc(OC(F)(F)F)c(F)c2)CCOc2cccnc21)c1ccc(C(=O)C(F)(F)F)cc1 ; "TFA"]

### (S)—N-(4-(3-fluoro-4-(trifluoromethoxy)phenyl)-3,4-dihydro-2H-pyrano[3,2-b]pyridin-4-yl)-4-(2,2,2-trifluoroacetyl)benzamide 2,2,2-trifluoroacetate

To a solution of N—-((S)-4-(3-fluoro-4-(trifluoromethoxy)phenyl)-3,4-dihydro-2H-pyrano[3,2-b]pyridin-4-yl)-4-((R)-2,2,2-trifluoro-1-hydroxyethyl)benzamide and N—((S)-4-(3-fluoro-4-(trifluoromethoxy)phenyl)-3,4-dihydro-2H-pyrano[3,2-b]pyridin-4-yl)-4-((S)-2,2,2-trifluoro-1-hydroxyethyl)benzamide (1:1) (170 mg, 0.160 mmol) and DCM (10 mL), was added Dess-Martin periodinane (175 mg, 0.413 mmol). After 4 h, the reaction was treated with saturated aqueous NaHCO₃ (2 mL). After 1d, the reaction was diluted with water (10 mL) and extracted with 10% MeOH in DCM (3×10 mL). The combined organic layers were concentrated in vacuo, and the residue was adsorbed onto a plug of silica gel and chromatographed through a Redi-Sep® pre-packed silica gel column (4 g), eluting with 0-50% EtOAc in hexanes, to provide impure product. The material was repurified by reverse-phase preparative HPLC (20% to 80% MeCN (0.1% TFA) in water (0.1% TFA)) to afford the title compound as a white solid after lyophilization. MS (ESI pos. ion) m/z: 547.1 (M+H₂O). ¹H NMR (300 MHz, MeOH-d₄) δ: 8.24 (dd, J=4.6, 1.5 Hz, 1H), 7.69-7.90 (m, 4H), 7.36-7.57 (m, 4H), 7.23 (d, J=8.8 Hz, 1H), 4.41-4.53 (m, 1H), 4.12-4.26 (m, 1H), 3.19 (dd, J=9.1, 3.6 Hz, 1H), 2.90-3.03 (m, 1H).

### Example 161

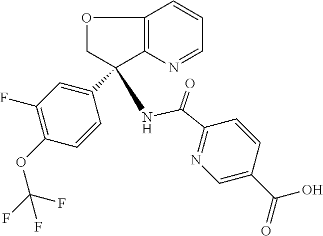 [SMILES: O=C(O)c1ccc(C(=O)N[C@@]2(c3ccc(OC(F)(F)F)c(F)c3)COc3cccnc32)nc1]

### (R)-6-((3-(3-Fluoro-4-(trifluoromethoxy)phenyl)-2,3-dihydrofuro[3,2-b]pyridin-3-yl)carbamoyl)nicotinic acid

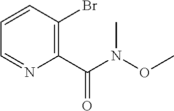

Step 1: 3-bromo-N-methoxy-N-methylpicolinamide

To a solution of 3-bromo-2-pyridinecarboxylic acid (1.97 g, 9.75 mmol) in DCM (24 mL) at 0° C. was added 1,1′-carbonyldiimidazole (1.74 g, 10.73 mmol). The reaction mixture was warmed to room temperature and stirred for 3 h, cooled to 0° C. and TEA (1.65 ml, 11.84 mmol) and N,O-dimethylhydroxylamine hydrochloride (1.04 g, 10.66 mmol) were added. The reaction mixture was warmed to room temperature and stirred for 16 h and partially concentrated. The reaction mixture was then diluted with EtOAc and water. The aqueous phase was extracted with EtOAc (6×) and the combined organic layers were washed with brine, dried over MgSO₄, filtered, and concentrated. Purification by flash column chromatography on silica gel (50-100% EtOAc in hexanes) gave the title compound as a white solid. MS (ESI pos. ion) m/z: 245.0, 247.0 (MH+).

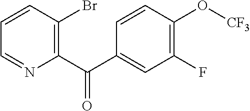 [SMILES: O=C(c1ccc(OC(F)(F)F)c(F)c1)c1ncccc1Br]

Step 2: (3-bromopyridin-2-yl)(3-fluoro-4-(trifluoromethoxy)phenyl)-methanone

To a solution of 4-bromo-2-fluoro-1-(trifluoromethoxy)benzene (3.30 g, 12.74 mmol) in THF (15 mL) at 0° C. was added isopropylmagnesium chloride (2M in Et₂O, 6.35 mL, 12.70 mmol). The reaction mixture was stirred at 0° C. for 2 h, warmed to room temperature and stirred for 18 h, cooled to 0° C. and added via cannula to a solution of 3-bromo-N-methoxy-N-methylpicolinamide (2.25 g, 9.18 mmol) in THF (5 mL) at 0° C. The reaction mixture was stirred at 0° C. for 30 min, warmed to room temperature and stirred for 4 h. The reaction was then cooled to 0° C. and quenched with saturated NH₄Cl (10 mL). The reaction mixture was next diluted with EtOAc (20 mL) and water (20 mL). The aqueous phase was extracted with EtOAc (2×10 mL), and the combined organic layers were washed with brine (20 mL), dried over MgSO₄, filtered, and concentrated. Purification by flash column chromatography on silica gel (5-10% EtOAc in hexanes) gave as a pale yellow oil. MS (ESI pos. ion) m/z: 364.0 (MH+).

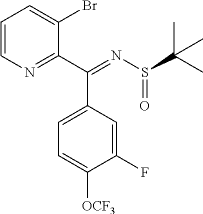 [SMILES: CC(C)(C)[S@@](=O)/N=C(\c1ccc(OC(F)(F)F)c(F)c1)c1ncccc1Br]

Step 3: (R,E)-N-((3-bromopyridin-2-yl)(3-fluoro-4-(trifluoromethoxy)-phenyl)methylene)-2-methylpropane-2-sulfinamide

To a RBF containing (3-bromopyridin-2-yl)(3-fluoro-4-(trifluoromethoxy)phenyl)methanone (10 g, 27.5 mmol), were added (R)-2-methylpropane-2-sulfinamide (5.045 g, 41.6 mmol) and titanium (IV) ethoxide (20.5 ml, 83 mmol). The solution was stirred at 70° C. After 24 h, the reaction was poured into brine (125 mL) and stirred for 5 min. The suspension was then diluted with EtOAc (50 mL) and stirred for a further 5 min. The suspension was filtered and the filtrate separated. The aqueous layer was extracted with EtOAc (2×20 mL). The combined EtOAc layers were concentrated in vacuo and adsorbed onto a plug of silica gel and chromatographed through a Redi-Sep® pre-packed silica gel column, eluting with 0-35% EtOAc in hexanes, to provide the title compound as a yellow solid. MS (ESI pos. ion) m/z: 466.9 (MH+).

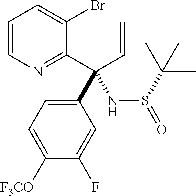

Step 4: (S)—N—((R)-1-(3-bromopyridin-2-yl)-1-(3-fluoro-4-(trifluoromethoxy)-phenyl)allyl)-2-methylpropane-2-sulfinamide

To a solution of (S,Z)—N-((3-bromopyridin-2-yl)(3-fluoro-4-(trifluoromethoxy)phenyl)methylene)-2-methylpropane-2-sulfinamide (Intermediate 2 g, 4.28 mmol) in 2-MeTHF (14 ml) at −78° C. was added vinylmagnesium chloride, (1.6M in THF, 4.55 ml, 7.28 mmol) dropwise over 5 min. The reaction mixture was stirred at −78° C. for 30 min and then was quenched with saturated NH₄Cl solution. The resulting mixture was warmed to room temperature and diluted with water and DCM. The aqueous phase was extracted with DCM (2×). The combined organic layers were washed with brine, dried over MgSO₄, filtered and concentrated in vacuo. Purification by flash chromatography (10-50% EtOAc in hexanes) afforded the title compound as a viscous colorless oil. MS (ESI pos. ion) m/z: 494.9 (MH+). ¹H NMR (400 MHz, DMSO-d₆) d: 8.68 (dd, J=4.60, 1.47 Hz, 1H), 8.11 (dd, J=8.02, 1.37 Hz, 1H), 7.48-7.55 (m, 1H), 7.37-7.45 (m, 2H), 6.85-7.00 (m, 2H), 6.35 (s, 1H), 5.34 (d, J=11.15 Hz, 1H), 4.85 (d, J=17.02 Hz, 1H), 1.19 (s, 9H).

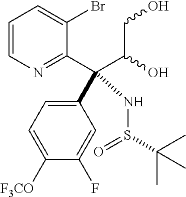

Step 5: (S)—N-((1R)-1-(3-bromopyridin-2-yl)-1-(3-fluoro-4-(trifluoromethoxy)phenyl)-2,3-dihydroxypropyl)-2-methylpropane-2-sulfinamide

To a solution of (S)—N—((R)-1-(3-bromopyridin-2-yl)-1-(3-fluoro-4-(trifluoromethoxy)phenyl)allyl)-2-methylpropane-2-sulfinamide (2.15 g, 4.34 mmol) in THF (10.8 ml) and water (1.8 ml) was added OsC₄ (4 wt. % solution in water, 1.326 ml, 0.217 mmol). The reaction was stirred at room temperature for 5 min, during which 4-methylmorpholine N-oxide monohydrate (1.027 g, 7.60 mmol) was added. The reaction was then heated at 50° C. for 2 h. The cooled reaction mixture was concentrated in vacuo, and the resulting residue was purified via flash chromatography (10-100% EtOAc in hexanes, followed by 1-5% MeOH in DCM) to afford the title compound as a light tan foam. MS (ESI pos. ion) m/z: 528.8, 530.9 (MH+). ¹H NMR (400 MHz, DMSO-d₆) δ: 8.72 (dd, J=4.50, 1.37 Hz, 1H), 8.04 (dd, J=8.02, 1.37 Hz, 1H), 7.15-7.56 (m, 3H), 6.29 (s, 1H), 5.29 (d, J=5.09 Hz, 1H), 4.83 (t, J=5.28 Hz, 1H), 4.64-4.72 (m, 1H), 3.73 (ddd, J=10.81, 5.33, 1.27 Hz, 1H), 2.58 (dt, J=11.00, 5.55 Hz, 1H), 1.22 (s, 9H).

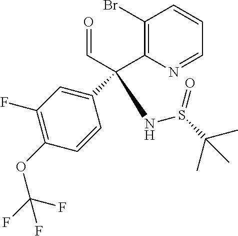 [SMILES: CC(C)(C)[S@@](=O)N[C@](C=O)(c1ccc(OC(F)(F)F)c(F)c1)c1ncccc1Br]

Step 6: (R)—N—((R)-1-(3-Bromopyridin-2-yl)-1-(3-fluoro-4-(trifluoro-methoxy)phenyl)-2-oxoethyl)-2-methylpropane-2-sulfinamide

N-((1R)-1-(3-bromopyridin-2-yl)-1-(3-fluoro-4-(trifluoromethoxy)phenyl)-2,3-dihydroxypropyl)-2-methylpropane-2-sulfinamide (1.84 g, 3.48 mmol) was dissolved in acetone (50 mL) and cooled in an ice bath. A solution of sodium periodate (1.2 g, 5.61 mmol) in water (15 mL) was added slowly, and the reaction was stirred for 1 h in a cooling bath. The reaction was allowed to warm to room temperature and stirred for 24 h. Water (200 mL) and EtOAc (300 mL) were added, and the phases were mixed and separated. The organic layer was dried with MgSO₄ and concentrated in vacuo. Purification using silica gel chromatography (EtOAc in hexanes) gave the title compound as a white solid. MS (ESI pos. ion) m/z: 497.9, 499.0 (MH+).

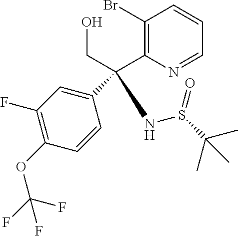 [SMILES: CC(C)(C)[S@@](=O)N[C@](CO)(c1ccc(OC(F)(F)F)c(F)c1)c1ncccc1Br]

Step 7: (R)—N—((R)-1-(3-Bromopyridin-2-yl)-1-(3-fluoro-4-(trifluoro-methoxy)phenyl)-2-hydroxyethyl)-2-methylpropane-2-sulfinamide

(R)—N—((R)-1-(3-Bromopyridin-2-yl)-1-(3-fluoro-4-(trifluoromethoxy)phenyl)-2-oxoethyl)-2-methylpropane-2-sulfinamide (0.598 g, 1.202 mmol) was dissolved in MeOH (75 mL) and treated with NaBH₄ (0.100 g, 2.64 mmol). The solution was stirred for 20 minutes after which water (10 mL) was added. The resulting mixture was stirred for another 20 minutes. The mixture was then concentrated in vacuo to ˜15 mL and then EtOAc (200 mL) and 0.5 N NaOH (100 mL) were added. The phases were mixed, separated, and the organic layer was washed with 50% saturated NaHCO₃ (100 mL) before drying with MgSO₄ and concentrating in vacuo. The title compound thus obtained was used without purification. MS (ESI pos. ion) m/z: 498.8, 500.9 (MH+).

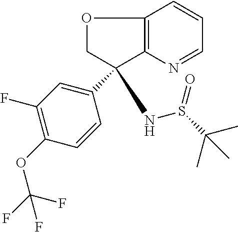 [SMILES: CC(C)(C)[S@@](=O)N[C@@]1(c2ccc(OC(F)(F)F)c(F)c2)COc2cccnc21]

Step 8: (R)—N—((R)-3-(3-Fluoro-4-(trifluoromethoxy)phenyl)-2,3-dihydrofuro[3,2-b]pyridin-3-yl)-2-methylpropane-2-sulfinamide

The (R)—N—((R)-1-(3-bromopyridin-2-yl)-1-(3-fluoro-4-(trifluoromethoxy)phenyl)-2-hydroxyethyl)-2-methylpropane-2-sulfinamide was combined with cesium carbonate (0.784 g, 2.405 mmol), quinolin-8-ol (0.0259 g, 0.178 mmol), and copper(I) iodide (0.0158 g, 0.083 mmol) in a microwave vial under argon.

Toluene (1 mL) was added, and the vial was sealed. The reaction mixture was then heated in a 90° C. oil bath for 60 h. The reaction product was dissolved in EtOAc (200 mL) and washed with water (100 mL). The organic layer was dried with MgSO₄ and concentrated in vacuo. Purification using silica chromatography (EtOAc in hexanes) gave the title compound. MS (ESI pos. ion) m/z: 418.9 (MH+).

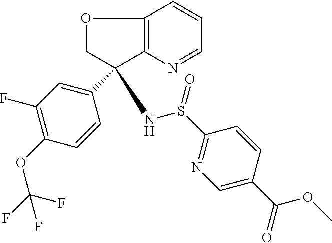 [SMILES: COC(=O)c1ccc(S(=O)N[C@@]2(c3ccc(OC(F)(F)F)c(F)c3)COc3cccnc32)nc1]

Step 9: (R)-Methyl 6-((3-(3-fluoro-4-(trifluoromethoxy)phenyl)-2,3-dihydrofuro[3,2-b]pyridin-3-yl)carbamoyl)nicotinate

(R)—N—((R)-3-(3-fluoro-4-(trifluoromethoxy)phenyl)-2,3-dihydrofuro[3,2-b]pyridin-3-yl)-2-methylpropane-2-sulfinamide (0.080 g, 0.191 mmol) was dissolved in a mixture of DCM (2 mL) and EtOH (2 mL) and treated with HCl (1M in Et₂O, 1 mL, 1 mmol). The mixture was stirred for 30 min and then concentrated in vacuo. The residue was dissolved in dry DMF (3 mL) and treated with 5-(methoxycarbonyl)-picolinic acid (0.020 g, 0.110 mmol) and HATU (0.050 g, 0.131 mmol). DIPEA (0.250 ml, 1.437 mmol) was added last, and the reaction was stirred at room temperature. After 20 min, water (50 mL) and DCM (50 mL) were added. The phases were mixed and separated. The organic layer was washed with 25% saturated NH₄Cl (50 mL) followed by 50% saturated NaHCO₃ (50 mL) before drying with MgSO₄ and concentrated in vacuo. The residue was purified using silica chromatography (EtOAc in hexanes) to give the title compound. MS (ESI pos. ion) m/z: 478.0 (MH+).

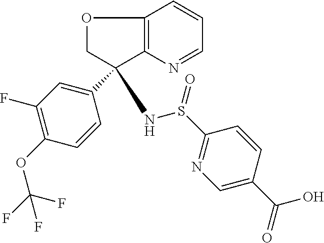 [SMILES: O=C(O)c1ccc(S(=O)N[C@@]2(c3ccc(OC(F)(F)F)c(F)c3)COc3cccnc32)nc1]

Step 10: (R)-6-((3-(3-Fluoro-4-(trifluoromethoxy)phenyl)-2,3-dihydrofuro[3,2-b]pyridin-3-yl)carbamoyl)nicotinic acid. (R)-Methyl 6-((3-(3-fluoro-4-(trifluoromethoxy)phenyl)-2,3-dihydrofuro[3,2-b]pyridin-3-yl)carbamoyl)nicotinate (0.044 g, 0.092 mmol) was dissolved in MeOH (20 mL) and treated with LiOH monohydrate (0.065 g, 1.549 mmol). Water (5 mL) was added, and the reaction was stirred at room temperature. After 90 min, the hydrolysis was complete, and the reaction was neutralized with 5N HCl (310 uL). The mixture was concentrated in vacuo, and the residue was purified using silica chromatography (0-10% MeOH in DCM) to give the title compound. MS (ESI pos. ion) m/z: 463.9 (MH+). ¹H NMR (DMSO-d₆) δ: 9.37 (s, 1H), 9.18 (s, 1H), 8.39-8.58 (m, 1H), 8.10-8.36 (m, 2H), 7.19-7.41 (m, 4H), 5.55 (d, J=10.76 Hz, 1H), 5.15 (d, J=10.76 Hz, 1H).

### Example 162

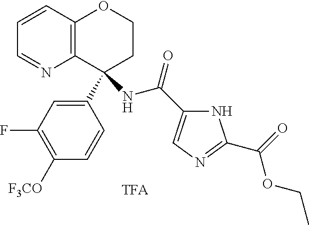 [SMILES: CCOC(=O)c1ncc(C(=O)N[C@]2(c3ccc(OC(F)(F)F)c(F)c3)CCOc3cccnc32)[nH]1]

### (S)-ethyl 5-(4-(3-fluoro-4-(trifluoromethoxy)phenyl)-3,4-dihydro-2H-pyrano[3,2-b]pyridin-4-yl)carbamoyl)-1H-imidazole-2-carboxylate 2,2,2-trifluoroacetate

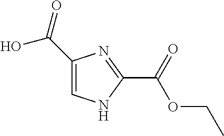

Step 1: 2-(ethoxycarbonyl)-1H-imidazole-4-carboxylic acid

To a vial containing 4-tert-butyl 2-ethyl 1H-imidazole-2,4-dicarboxylate (75 mg, 0.312 mmol) in DCM (2 mL) was added TFA (2 mL). The reaction was stirred at room temperature under N₂ for 2 h. The reaction was concentrated in vacuo, treated with MeOH (1 mL), and the solid was collected by filtration to give the title compound as a white solid which was used without purification in the next step. MS (ESI, positive ion) m/z: 185.0 (MH+).

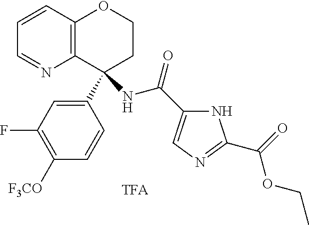 [SMILES: CCOC(=O)c1ncc(C(=O)N[C@]2(c3ccc(OC(F)(F)F)c(F)c3)CCOc3cccnc32)[nH]1]

Step 2: (S)-ethyl 5-((4-(3-fluoro-4-(trifluoromethoxy)phenyl)-3,4-dihydro-2H-pyrano[3,2-b]pyridin-4-yl)carbamoyl)-1H-imidazole-2-carboxylate 2,2,2-trifluoroacetate

To a vial with 2-(ethoxycarbonyl)-1H-imidazole-4-carboxylic acid (58 mg, 0.315 mmol) were added (S)-4-(3-fluoro-4-(trifluoromethoxy)-phenyl)-3,4-dihydro-2H-pyrano[3,2-b]pyridin-4-amine hydrochloride (96 mg, 0.262 mmol) and DMF (1 mL) followed by HATU (100 mg, 0.262 mmol) and TEA (0.110 mL, 0.787 mmol). The vial was sealed, and the mixture was stirred for 24 h at room temperature under air. The reaction was then diluted with water (10 mL) and EtOAc (5 mL), and the organic layer was separated. The aqueous layer was extracted with EtOAc (2×5 mL), and the organic layers were combined, dried (MgSO₄), and concentrated to give a residual brown oil. Purification by preparative HPLC (Gemini Axia 50×250 mm C18, 10%-100% MeCN 0.1% TFA/H₂O 0.1% TFA) gave the title compound as a white solid. MS (ESI, positive ion) m/z: 495.0 (MH+). ¹H NMR (300 MHz, DMSO-d₆) δ: 8.56 (s, 1H), 8.29 (t, J=2.9 Hz, 1H), 7.83 (s, 1H), 7.46-7.62 (m, 2H), 7.39 (d, J=2.8 Hz, 2H), 7.25 (d, J=8.6 Hz, 1H), 4.27-4.46 (m, 3H), 4.07 (t, J=9.9 Hz, 1H), 2.86-3.03 (m, 1H), 1.31 (t, J=7.1 Hz, 3H).

### Example 163

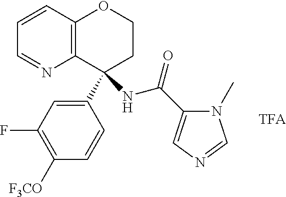 [SMILES: Cn1cncc1C(=O)N[C@]1(c2ccc(OC(F)(F)F)c(F)c2)CCOc2cccnc21]

### (S)—N-(4-(3-fluoro-4-(trifluoromethoxy)phenyl)-3,4-dihydro-2H-pyrano[3,2-b ]pyridin-4-yl)-1-methyl-1H-imidazole-5-carboxamide 2,2,2-trifluoroacetate

A microwave vial containing (S)-2-bromo-N-(4-(3-fluoro-4-(trifluoromethoxy)-phenyl)-3,4-dihydro-2H-pyrano[3,2-b]pyridin-4-yl)-1-methyl-1H-imidazole-5-carboxamide (50 mg, 0.097 mmol), (9,9-dimethyl-9H-xanthene-4,5-diyl)bis(diphenylphosphine) (5.6 mg, 9.70 mmol), K₂CO₃ (53.6 mg, 0.388 mmol), and bis(tri-t-butylphosphine)palladium (0) (9.9 mg, 0.019 mmol) was flushed with N₂ (3×), and then DMF (1 mL) and water (17.5 μL, 0.970 mmol) were added. The reaction was flushed with CO gas (3×) and CO was bubbled through the suspension 5 min. The reaction was ten heated at 80° C. in an oil bath and stirred under CO (balloon) for 19 h. The reaction mixture was filtered through Celite® brand filter agent and purified by preparative HPLC (Gemini Axia 50×250 mm C18, 10%-100% MeCN 0.1% TFA/H₂O 0.1% TFA) to give the title compound as a white solid. MS (ESI, positive ion) m/z: 437.0 (MH+). ¹H NMR (300 MHz, DMSO-d₆) δ: 9.00 (s, 1H), 8.59 (br. s., 1H), 8.12 (dd, J=3.9, 1.8 Hz, 1H), 8.08 (s, 1H), 7.41-7.60 (m, 2H), 7.23-7.35 (m, 2H), 7.20 (d, J=9.8 Hz, 1H), 4.24-4.42 (m, 1H), 4.01-4.19 (m, 2H), 3.24 (dd, J=14.5, 7.7 Hz, 1H), 2.68-2.90 (m, 1H).

### Intermediate 37

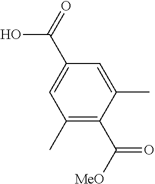

### 4-(methoxycarbonyl)-3,5-dimethylbenzoic acid

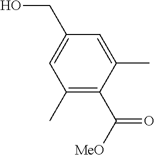

Step 1: methyl 4-(hydroxymethyl)-2,6-dimethylbenzoate

An ace pressure tube (100 mL) with methyl isodehydroacetate (1.00 g, 5.49 mmol) and prop-2-yn-1-ol (4.0 mL, 68.9 mmol) was sealed, and heated to 180° C. in a sand bath for 3 h.

The reaction was cooled to room temperature. The residue was dissolved in DCM and purified by flash chromatography (0-40% EtOAc in hexanes) to give the title compound as a waxy yellow solid. ¹H NMR (300 MHz, DMSO-d₆) δ: 7.02 (s, 2H), 5.21 (t, J=5.7 Hz, 1H), 4.44 (d, J=5.7 Hz, 2H), 3.78-3.88 (m, 3H), 2.22 (s, 6H).

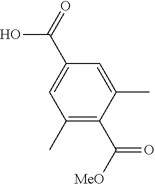

Step 2: 4-(methoxycarbonyl)-3,5-dimethylbenzoic acid. To a solution of methyl 4-(hydroxymethyl)-2,6-dimethylbenzoate (500 mg, 2.57 mmol) in CH₃CN (5 mL), water (7.5 mL), and EtOAc (5 mL) were added sodium periodate (2258 mg, 10.55 mmol) and ruthenium (III) chloride (30 mg, 0.145 mmol). The reaction was stirred at room temperature for 4 h. The reaction was diluted with H₂O (50 mL) and extracted with EtOAc (2×100 mL). The combined organic layers were dried (MgSO₄), and concentrated. Purification by flash chromatography (10-40% 1% AcOH in EtOAc in hexanes) gave the title compound as a white solid. ¹H NMR (300 MHz, CDCl₃) δ: 7.80 (s, 2H), 3.96 (s, 3H), 2.38 (s, 6H).

### Intermediate 38

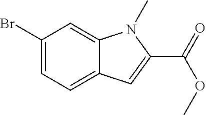

### methyl 6-bromo-1-methyl-1H-indole-2-carboxylate

To a solution of 6-bromo-1H-indole-2-carboxylic acid (500 mg, 2.083 mmol) and DMF (7 mL) was added K₂CO₃ (725 mg, 5.25 mmol) and methyl iodide (0.28 mL, 4.48 mmol). The reaction was stirred at room temperature. After 1 h, LC-MS shows mono alkylation as major component, the reaction was stirred in a 60° C. oil bath. After 24 h, the reaction was allowed to cool to room temperature. After a further 36 h, the reaction was diluted with water (25 mL) and after stirring for 30 min, the solution was filtered and rinsed with water (15 mL). The solids were dried in the funnel to give the title compound as a white solid.

### Intermediate 39

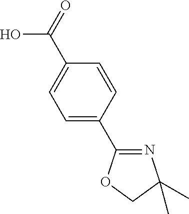 [SMILES: CC1(C)COC(c2ccc(C(=O)O)cc2)=N1]

### 4-(4,4-dimethyl-4,5-dihydrooxazol-2-yl)benzoic acid

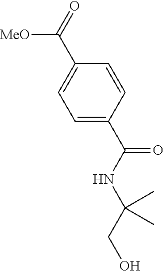 [SMILES: COC(=O)c1ccc(C(=O)NC(C)(C)CO)cc1]

Step 1: methyl 4-((1-hydroxy-2-methylpropan-2-yl)carbamoyl)benzoate

To a solution of 4-(methoxycarbonyl)benzoic acid (2.0 g, 11.10 mmol) in DMF (25 mL) under N₂ was added 1,1′-carbonyldiimidazole (1.890 g, 11.66 mmol). The solution was stirred at room temperature under N₂ for 1 h. 2-Amino-2-methylpropan-1-ol (1.187 g, 13.32 mmol) was added, and the reaction was stirred overnight at room temperature. The reaction was diluted with saturated aqueous NaHCO₃, and extracted with DCM (3×50 mL). The combined organic layers were washed with brine (50 mL), dried (MgSO₄), and concentrated in vacuo. Purification by flash chromatography (10-50% EtOAc in hexanes) gave the title compound contaminated with DMF ˜50 wt %. The product was carried on to the next step without further purification.

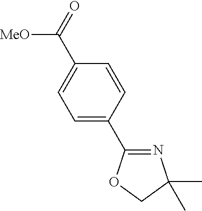 [SMILES: COC(=O)c1ccc(C2=NC(C)(C)CO2)cc1]

Step 2: methyl 4-(4,4-dimethyl-4,5-dihydrooxazol-2-yl)benzoate

To a solution of methyl 4-((1-hydroxy-2-methylpropan-2-yl)carbamoyl)benzoate (2.0 g, 7.96 mmol) in CHCl₃ (40 mL) was added thionyl chloride (0.695 mL, 9.55 mmol). The mixture was heated to 60° C. in a sealed tube for 24 h. The reaction was quenched by addition to a rapidly stirring solution of saturated aqueous NaHCO₃ (250 mL) and was then stirred for 1 h. The mixture was extracted with DCM (3×100 mL), and the organic layers were combined, washed with brine (100 mL), dried (MgSO₄), and concentrated in vacuo. Purification by flash column chromatography (20-80% EtOAc in hexanes) gave the title compound as a white solid. MS (ESI, positive ion) m/z: 234.1 (MH+). ¹H NMR (300 MHz, CDCl₃) δ: 7.90-8.15 (m, 4H), 4.14 (s, 2H), 3.94 (s, 3H), 1.40 (s, 6H).

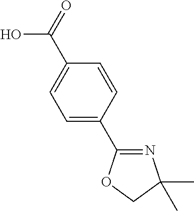 [SMILES: CC1(C)COC(c2ccc(C(=O)O)cc2)=N1]

Step 3: 4-(4,4-dimethyl-4,5-dihydrooxazol-2-yl)benzoic acid

Methyl 4-(4,4-dimethyl-4,5-dihydrooxazol-2-yl)benzoate (75 mg, 0.322 mmol) was dissolved in THF (1 mL), and MeOH (0.5 mL) and LiOH (2M in H₂O, 0.322 mL, 0.64 mmol) were added. The resulting mixture was stirred at room temperature for 18 h. The reaction was quenched with 1N aqueous HCl (0.64 mL) and concentrated to give the title compound as a white solid which was used in the next step without further purification. MS (ESI, positive ion) m/z: 220.1 (MH+).

### Intermediate 40

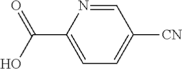 [SMILES: N#Cc1ccc(C(=O)O)nc1]

### 5-cyanopicolinic acid

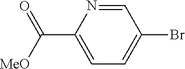 [SMILES: COC(=O)c1ccc(Br)cn1]

Step 1: methyl 5-bromopicolinate

To a solution of 5-bromopicolinic acid (3.39 g, 16.78 mmol) in MeOH (10 mL) was added H₂SO₄ (0.045 mL, 0.839 mmol) (concentrated, 97 wt %). The mixture was heated to reflux and stirred under N₂ for 4 h (low conversion was observed). The reaction was cooled to rt, and additional concentrated H₂SO₄ (0.939 mL, 17.62 mmol) was added slowly (tan suspension turned into a brown solution). The reaction was again heated to reflux and stirred for 18 h under N₂. The reaction was next slowly poured into saturated aqueous NaHCO₃ (100 mL) and extracted with EtOAc (2×100 mL). The organic layers were combined, washed with saturated aqueous NaHCO₃, brine, dried (MgSO₄), and concentrated in vacuo to give the title compound which was used without further purification in the next step.

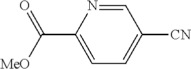 [SMILES: COC(=O)c1ccc(C#N)cn1]

Step 2: methyl 5-cyanopicolinate

A 250 mL RBF containing methyl 5-bromopicolinate (2.60 g, 12.04 mmol), tetrakis(triphenylphosphine)palladium (0) (0.556 g, 0.481 mmol), and zinc cyanide (0.886 mL, 13.97 mmol) was flushed with N₂ (3×) and then DMF (15 mL) was added. The reaction was immersed in a preheated 80° C. oil bath and stirred under N₂ for 18 h. The reaction was then cooled to room temperature, water was added, and the resulting solid was filtered and dried with vacuum suction. The compound was dissolved in DCM/EtOAc/MeOH, SiO₂ was added, and the solvent was removed in vacuo, the silica plug was purified by flash chromatography (0-100% DCM in EtOAc) to give the title compound as a light yellow solid.

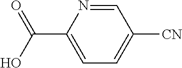 [SMILES: N#Cc1ccc(C(=O)O)nc1]

Step 3: 5-cyanopicolinic acid

To a suspension of methyl 5-cyanopicolinate (122 mg, 0.752 mmol) in THF (4 mL) and MeOH (2 mL) was added LiOH (2 M in H₂O, 0.82 mL, 1.64 mmol). The resulting reaction was stirred at room temperature for 24 h. The reaction was then neutralized with 10% aqueous citric acid (3 mL), and extracted with EtOAc (2×5 mL). The combined organic layers were washed with brine (5 mL), dried (MgSO₄), and concentrated to give the title compound as a yellow solid which was used in the next step without further purification.

### Intermediate 41

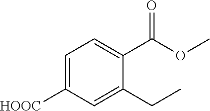

### 3-ethyl-4-(methoxycarbonyl)benzoic acid

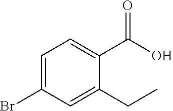

Step 1: 4-bromo-2-ethylbenzoic acid

To a stirred solution of 4-bromo-2-fluorobenzoic acid (10 g, 45.7 mmol) in THF (100 mL) at −78° C. was added ethylmagnesium bromide (91 ml, 91 mmol) dropwise. After the addition, the mixture was gradually warmed to room temperature and stirred overnight. The reaction was recooled to −78° C. and slowly quenched with 5N HCl. The mixture was then warmed to room temperature and extracted with DCM (3×). The combined extracts were dried over Na₂SO₄, concentrated and purified by flash chromatography (0-40% EtOAc in hexanes) to give the title compound as an off white solid. MS (ESI, positive ion) m/z: 230.0 (MH+). ¹H NMR (400 MHz, CDCl₃) δ: 7.90 (d, J=8.4 Hz, 1H), 7.47 (d, J=2.0 Hz, 1H), 7.42 (dd, J=8.4, 2.0 Hz, 1H), 3.04 (q, J=7.4 Hz, 2H), 1.26 (t, J=7.5 Hz, 3H).

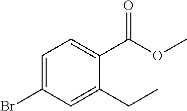

Step 2: methyl 4-bromo-2-ethylbenzoate

To a stirred mixture of 4-bromo-2-ethylbenzoic acid (19.58 g, 85 mmol) and K₂CO₃ (35.4 g, 256 mmol) in DMF (100 mL) was added iodomethane (8.02 mL, 128 mmol). After the addition, the mixture was stirred for 2 h, diluted with EtOAc, washed with H₂O, dried over MgSO₄, and concentrated to give the title compound as a yellow oil. ¹H NMR (400 MHz, CDCl₃) δ: 7.73 (d, J=8.4 Hz, 1H), 7.43 (d, J=2.0 Hz, 1H), 7.37 (dd, J=8.3, 2.1 Hz. 1H), 3.89 (s, 3H), 2.96 (q, J=7.4 Hz, 2H), 1.23 (t, J=7.4 Hz, 3H).

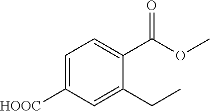

Step 3: 3-ethyl-4-(methoxycarbonyl)benzoic acid

CO gas was bubbled through a mixture of methyl 4-bromo-2-ethylbenzoate (5 g, 20.57 mmol), (9,9-dimethyl-9H-xanthene-4,5-diyl)bis(diphenylphosphine) (0.595 g, 1.028 mmol), palladium acetate (0.231 g, 1.028 mmol), and TEA (14.26 mL, 103 mmol) in THF (100 mL) and water (50 mL) for 15 min. The reaction was then heated at 60° C. under a CO balloon for 16 h. The reaction mixture was cooled, diluted with H₂O, and extracted with ether (discarded). The aqueous layer was acidified, and the tan solid was collected, dried and used in the next step. ¹H NMR (400 MHz, CDCl₃) δ: 8.03 (d, J=1.4 Hz, 1H), 7.97 (dd, J=8.1, 1.7 Hz, 1H), 7.90 (d, J=8.2 Hz, 1H), 3.94 (s, 3H), 3.02 (q, J=7.5 Hz, 2H), 1.28 (t, J=7.5 Hz, 3H).

### Intermediate 42

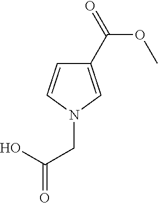

### 2-(3-(methoxycarbonyl)-1H-pyrrol-1-yl)acetic acid

A mixture of methyl 1H-pyrrole-3-carboxylate (1.12 g, 8.95 mmol), tert-butyl bromoacetate (1.734 ml, 10.74 mmol), and cesium carbonate (4.37 g, 13.43 mmol) in DMF (10 mL) was stirred at room temperature for 16 h. H₂O was added, and the mixture was extracted with DCM (3×). The extracts were dried over Na₂SO₄, concentrated, dissolved in MeOH (20 mL), then added H₂O (10 mL) and LiOH (0.429 g, 17.90 mmol). The reaction mixture was then stirred at room temperature in 3 h, concentrated, diluted with H₂O cooled in an ice bath, acidified with 2N HCl, extracted with DCM (3×). The extracts were dried over Na₂SO₄, concentrated to dryness to give the title compound. MS (ESI pos. ion) m/z: 184 (MH+). ¹H NMR (400 MHz, DMSO-d₆) δ: 13.02 (br. s., 1H), 7.43 (t, J=1.9 Hz, 1H), 6.80 (t, J=2.5 Hz, 1H), 6.38 (dd, J=2.8, 1.7 Hz, 1H), 4.73-4.88 (m, 2H), 3.61-3.75 (m, 3H).

### General Amide Formation Procedure for Examples 164-280 and 314

To a solution of an amine or amine hydrochloride, the corresponding carboxylic acid (1.2 eq), and DIPEA (2 eq) in DCM or DMF (1 mL) at room temperature, was added an amide coupling reagent such as (HATU, TBTU, T3P, or EDCI) (1.2 eq.). The reaction was stirred for 1-16 h at room temperature. The reaction was then purified by one of the following methods: Method A: The reaction was diluted with DMF (1 mL), filtered through a syringe filter, and then purified by preparative reverse phase HPLC (gradient elution 10-100% MeCN/0.1% TFA in H₂O). The product-containing fractions were then combined and the solvent was removed by lyophilization to provide the target compound as the TFA salt; Method B: The reaction was diluted with DMF (1 mL), filtered through a syringe filter, and then purified by preparative reverse phase HPLC (gradient elution 10-100% MeCN/0.1% TFA in H₂O). The product-containing fractions were combined and concentrated, and the resulting product was dissolved in MeOH (1 mL) and washed through StratoSpheres® PL-HCO₃ MP-resin, and the resin was further washed with MeOH (2×0.4 mL). The combined filtrates were then concentrated and dried in vacuo to give the title compounds as free bases; Method C: After purification by reverse phase HPLC the product-containing fractions were concentrated, the solids dissolved in DCM, and the organic layer was extracted with saturated aqueous NaHCO₃, dried, and concentrated to provide the title compounds as free bases. Method D: The reaction mixture was diluted with H₂O and extracted with EtOAc (3×). The combined organic layers were dried (MgSO₄) and concentrated. Purification by silica flash chromatography provided the title compounds as free bases. Method E: The reactions were filtered and purified by mass directed preparative reverse phase HPLC (gradient elution 10-100% MeCN/0.1% formic acid in H₂O/0.1% formic acid). The product-containing fractions were combined and concentrated to provide the title compounds as formic acid salts.

TABLE 14
Examples 164-280 and 314 prepared via amide coupling analogous to general procedures described above.

| | Amine | | Product | Product | MS |
|---|---|---|---|---|---|
| Ex # | Intermediate | Acid Structure | Structure | Name | M + H |
| 164 | 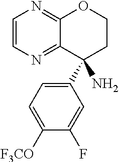 [SMILES: N[C@]1(c2ccc(OC(F)(F)F)c(F)c2)CCOc2nccnc21] | 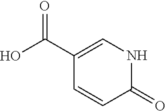 | 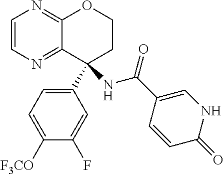 [SMILES: O=C(N[C@]1(c2ccc(OC(F)(F)F)c(F)c2)CCOc2nccnc21)c1ccc(=O)[nH]c1] | (S)-N-(8-(3- fluoro-4- (trifluoromethoxy) phenyl)- 7,8-dihydro-6H- pyrano[2,3-b] pyrazin-8-yl)- 6-oxo-1,6- dihydropyridine- 3-carboxamide | 450.8 |
| 165 | 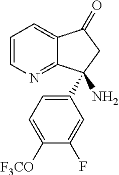 [SMILES: N[C@]1(c2ccc(OC(F)(F)F)c(F)c2)CC(=O)c2cccnc21] | 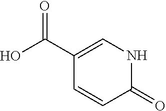 | 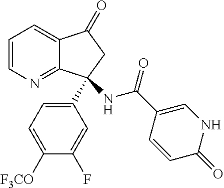 [SMILES: O=C(N[C@]1(c2ccc(OC(F)(F)F)c(F)c2)CC(=O)c2cccnc21)c1ccc(=O)[nH]c1] | (S)-N-(7-(3- fluoro-4- (trifluoromethoxy) phenyl)- 5-oxo-6,7- dihydro-5H- cyclopenta[b] pyridin-7-yl)- 6-oxo-1,6- dihydropyridine- 3-carboxamide | 447.8 |
| 166 | 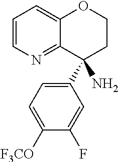 [SMILES: N[C@]1(c2ccc(OC(F)(F)F)c(F)c2)CCOc2cccnc21] | 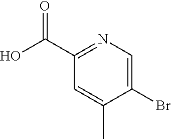 | 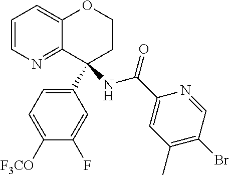 [SMILES: Cc1cc(C(=O)N[C@]2(c3ccc(OC(F)(F)F)c(F)c3)CCOc3cccnc32)ncc1Br] | (S)-5-bromo- N-(4-(3- fluoro-4- (trifluoromethoxy) phenyl)- 3,4-dihydro- 2H-pyrano[3,2-b] pyridin-4- yl)-4- methylpicolin amide | 526.7 |
| 167 | 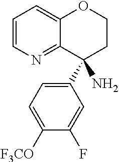 [SMILES: N[C@]1(c2ccc(OC(F)(F)F)c(F)c2)CCOc2cccnc21] | 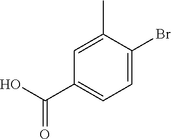 | 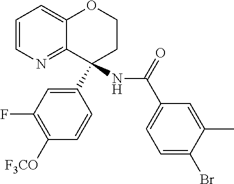 [SMILES: Cc1cc(C(=O)N[C@]2(c3ccc(OC(F)(F)F)c(F)c3)CCOc3cccnc32)ccc1Br] | (S)-4-bromo- N-(4-(3- fluoro-4- (trifluoromethoxy) phenyl)- 3,4-dihydro- 2H-pyrano [3,2-b]pyridin- 4-yl)-3- methylbenzamide | 524.9, 526.8 |
| 168 | 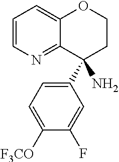 [SMILES: N[C@]1(c2ccc(OC(F)(F)F)c(F)c2)CCOc2cccnc21] | 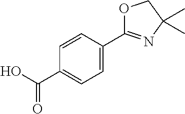 [SMILES: CC1(C)COC(c2ccc(C(=O)O)cc2)=N1] | 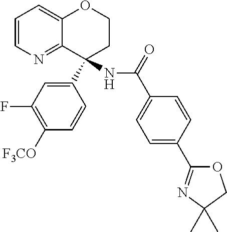 [SMILES: CC1(C)COC(c2ccc(C(=O)N[C@]3(c4ccc(OC(F)(F)F)c(F)c4)CCOc4cccnc43)cc2)=N1] | (S)-4-(4,4- dimethyl-4,5- dihydrooxazol- 2-yl)-N-(4- (3-fluoro-4- (trifluoromethoxy) phenyl)- 3,4-dihydro- 2H- pyrano[3,2- b]pyridin-4- yl)benzamide | 530.0 |
| 169 | 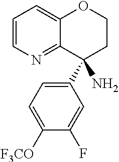 [SMILES: N[C@]1(c2ccc(OC(F)(F)F)c(F)c2)CCOc2cccnc21] | 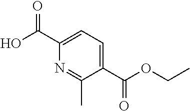 | 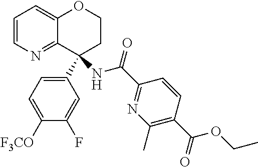 [SMILES: CCOC(=O)c1ccc(C(=O)N[C@]2(c3ccc(OC(F)(F)F)c(F)c3)CCOc3cccnc32)nc1C] | (S)-ethyl 6- ((4-(3-fluoro-4- (trifluoromethoxy) phenyl)- 3,4-dihydro- 2H-pyrano[3,2-b] pyridin-4- yl)carbamoyl)-2- methylnicotinate | 519.8 |
| 170 | 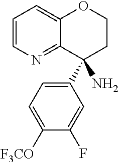 [SMILES: N[C@]1(c2ccc(OC(F)(F)F)c(F)c2)CCOc2cccnc21] | 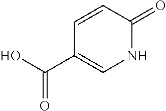 | 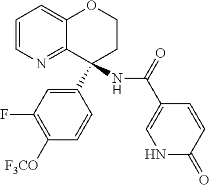 [SMILES: O=C(N[C@]1(c2ccc(OC(F)(F)F)c(F)c2)CCOc2cccnc21)c1ccc(=O)[nH]c1] | (S)-N-(4-(3- fluoro-4- (trifluoromethyl) phenyl)- 3,4-dihydro-2H- pyrano[3,2-b] pyridin-4- yl)-6-oxo-1,6- dihydropyridine- 3-carboxamide | 434.0 |
| 171 | 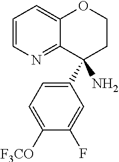 [SMILES: N[C@]1(c2ccc(OC(F)(F)F)c(F)c2)CCOc2cccnc21] | 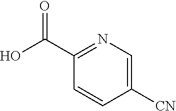 [SMILES: N#Cc1ccc(C(=O)O)nc1] | 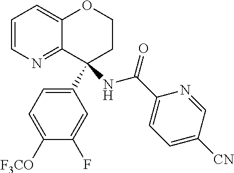 [SMILES: N#Cc1ccc(C(=O)N[C@]2(c3ccc(OC(F)(F)F)c(F)c3)CCOc3cccnc32)nc1] | (S)-5-cyano- N-(4-(3- fluoro-4- (trifluoromethoxy) phenyl)- 3,4-dihydro- 2H-pyrano[3,2-b] pyridin-4-yl) picolinamide | 458.9 |
| 172 | 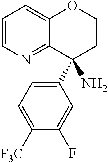 [SMILES: N[C@]1(c2ccc(C(F)(F)F)c(F)c2)CCOc2cccnc21] | 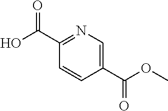 | 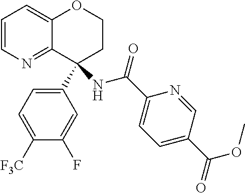 [SMILES: COC(=O)c1ccc(C(=O)N[C@]2(c3ccc(C(F)(F)F)c(F)c3)CCOc3cccnc32)nc1] | (S)-methyl 6- ((4-(3-fluoro-4- (trifluoromethyl) phenyl)- 3,4-dihydro- 2H-pyrano[3,2-b] pyridin-4-yl) carbamoyl) nicotinate | 476.0 |
| 173 | 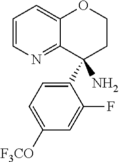 [SMILES: N[C@]1(c2ccc(OC(F)(F)F)cc2F)CCOc2cccnc21] | 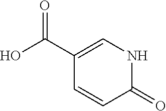 | 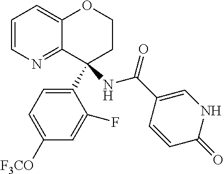 [SMILES: O=C(N[C@]1(c2ccc(OC(F)(F)F)cc2F)CCOc2cccnc21)c1ccc(=O)[nH]c1] | (S)-N-(4-(2- fluoro-4- (trifluoromethoxy) phenyl)- 3,4-dihydro- 2H-pyrano[3,2-b] pyridin-4-yl)- 6-oxo-1,6- dihydropyridine- 3-carboxamide | 450.0 |
| 174 | 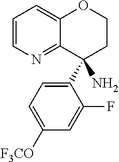 [SMILES: N[C@]1(c2ccc(OC(F)(F)F)cc2F)CCOc2cccnc21] | 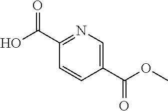 | 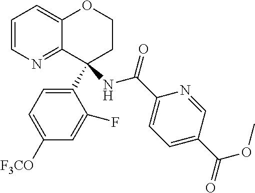 [SMILES: COC(=O)c1ccc(C(=O)N[C@]2(c3ccc(OC(F)(F)F)cc3F)CCOc3cccnc32)nc1] | (S)-methyl 6- ((4-(2-fluoro-4- (trifluoromethoxy) phenyl)- 3,4-dihydro- 2H-pyrano[3,2-b] pyridin-4-yl) carbamoyl) nicotinate | 492.0 |
| 175 | 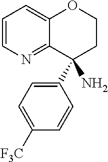 [SMILES: N[C@]1(c2ccc(C(F)(F)F)cc2)CCOc2cccnc21] | 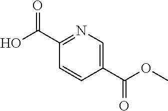 | 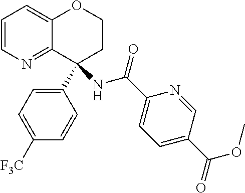 [SMILES: COC(=O)c1ccc(C(=O)N[C@]2(c3ccc(C(F)(F)F)cc3)CCOc3cccnc32)nc1] | (S)-methyl 6- ((4-(4- (trifluoromethyl) phenyl)- 3,4-dihydro- 2H-pyrano[3,2-b] pyridin-4-yl) carbamoyl) nicotinate | 458.0 |
| 176 | 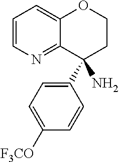 [SMILES: N[C@]1(c2ccc(OC(F)(F)F)cc2)CCOc2cccnc21] | 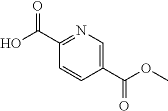 | 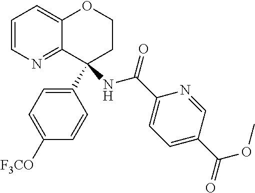 [SMILES: COC(=O)c1ccc(C(=O)N[C@]2(c3ccc(OC(F)(F)F)cc3)CCOc3cccnc32)nc1] | (S)-methyl 6- ((4-(4- (trifluoromethoxy) phenyl)- 3,4-dihydro- 2H-pyrano[3,2-b] pyridin-4-yl) carbamoyl) nicotinate | 474.0 |
| 177 | 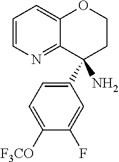 [SMILES: N[C@]1(c2ccc(OC(F)(F)F)c(F)c2)CCOc2cccnc21] | 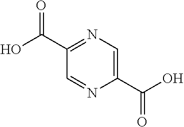 [SMILES: O=C(O)c1cnc(C(=O)O)cn1] | 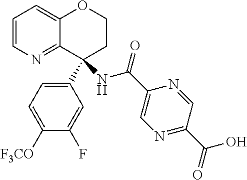 [SMILES: O=C(O)c1cnc(C(=O)N[C@]2(c3ccc(OC(F)(F)F)c(F)c3)CCOc3cccnc32)cn1] | (S)-5-((4-(3- fluoro-4- (trifluoromethoxy) phenyl)- 3,4-dihydro- 2H-pyrano[3,2-b] pyridin-4-yl) carbamoyl) pyrazine-2- carboxylic acid | 479.0 |
| 178 | 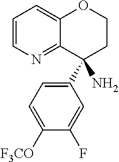 [SMILES: N[C@]1(c2ccc(OC(F)(F)F)c(F)c2)CCOc2cccnc21] | 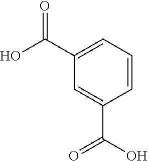 [SMILES: O=C(O)c1cccc(C(=O)O)c1] | 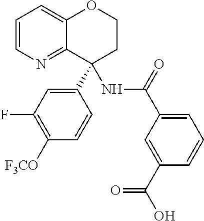 [SMILES: O=C(O)c1cccc(C(=O)N[C@]2(c3ccc(OC(F)(F)F)c(F)c3)CCOc3cccnc32)c1] | (S)-3-((4-(3- fluoro-4- (trifluoromethoxy) phenyl)- 3,4-dihydro- 2H-pyrano[3,2-b] pyridin-4- yl)carbamoyl) benzoic acid | 477.0 |
| Inter- mediate 43 | 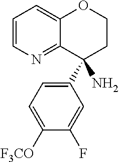 [SMILES: N[C@]1(c2ccc(OC(F)(F)F)c(F)c2)CCOc2cccnc21] | 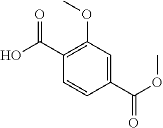 | 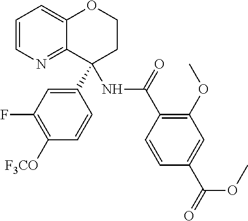 [SMILES: COC(=O)c1ccc(C(=O)N[C@]2(c3ccc(OC(F)(F)F)c(F)c3)CCOc3cccnc32)c(OC)c1] | (S)-methyl 4- ((4-(3-fluoro-4- (trifluoromethoxy) phenyl)- 3,4-dihydro- 2H-pyrano[3,2-b] pyridin-4-yl) carbamoyl)-3- methoxybenzoate | 521.0 |
| 179 | 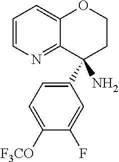 [SMILES: N[C@]1(c2ccc(OC(F)(F)F)c(F)c2)CCOc2cccnc21] | 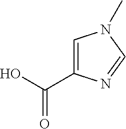 | 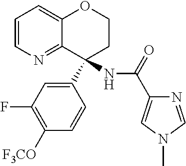 [SMILES: Cn1cnc(C(=O)N[C@]2(c3ccc(OC(F)(F)F)c(F)c3)CCOc3cccnc32)c1] | (S)-N-(4-(3- fluoro-4- (trifluoromethoxy) phenyl)- 3,4-dihydro- 2H-pyrano[3,2-b] pyridin-4- yl)-1-methyl- 1H-imidazole-4- carboxamide | 437.2 |
| 180 | 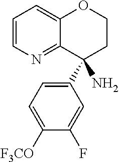 [SMILES: N[C@]1(c2ccc(OC(F)(F)F)c(F)c2)CCOc2cccnc21] | 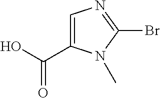 | 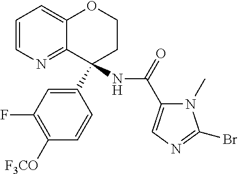 [SMILES: Cn1c(C(=O)N[C@]2(c3ccc(OC(F)(F)F)c(F)c3)CCOc3cccnc32)cnc1Br] | (S)-2-bromo- N-(4-(3- fluoro-4- (trifluoromethoxy) phenyl)- 3,4-dihydro- 2H-pyrano[3,2-b] pyridin-4-yl)-1- methyl-1H- imidazole-5- carboxamide | 515.0, 517.0 |
| 181 | 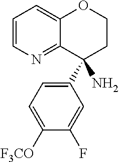 [SMILES: N[C@]1(c2ccc(OC(F)(F)F)c(F)c2)CCOc2cccnc21] | 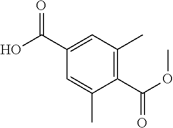 | 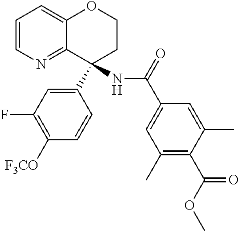 [SMILES: COC(=O)c1c(C)cc(C(=O)N[C@]2(c3ccc(OC(F)(F)F)c(F)c3)CCOc3cccnc32)cc1C] | (S)-methyl 4- ((4-(3-fluoro-4- (trifluoromethoxy) phenyl)- 3,4-dihydro- 2H-pyrano[3,2-b] pyridin-4-yl) carbamoyl)-2,6- dimethylbenzoate | 518.9 |
| 182 | 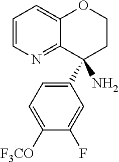 [SMILES: N[C@]1(c2ccc(OC(F)(F)F)c(F)c2)CCOc2cccnc21] | 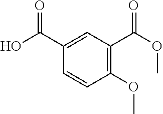 | 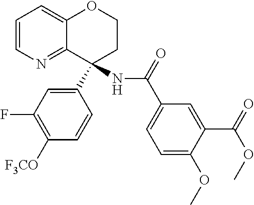 [SMILES: COC(=O)c1cc(C(=O)N[C@]2(c3ccc(OC(F)(F)F)c(F)c3)CCOc3cccnc32)ccc1OC] | (S)-methyl 5- ((4-(3-fluoro-4- (trifluoromethoxy) phenyl)- 3,4-dihydro- 2H-pyrano[3,2-b] pyridin-4-yl) carbamoyl)-2- methoxybenzoate | 521.0 |
| Inter- mediate 44 | 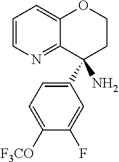 [SMILES: N[C@]1(c2ccc(OC(F)(F)F)c(F)c2)CCOc2cccnc21] | 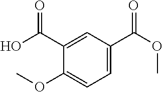 | 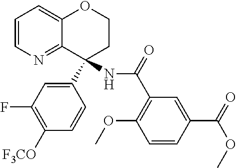 [SMILES: COC(=O)c1ccc(OC)c(C(=O)N[C@]2(c3ccc(OC(F)(F)F)c(F)c3)CCOc3cccnc32)c1] | (S)-methyl 3- ((4-(3-fluoro-4- (trifluoromethoxy) phenyl)- 3,4-dihydro- 2H-pyrano[3,2-b] pyridin-4-yl) carbamoyl)-4- methoxybenzoate | 521.0 |
| Inter- mediate 45 | 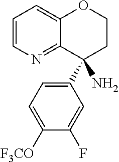 [SMILES: N[C@]1(c2ccc(OC(F)(F)F)c(F)c2)CCOc2cccnc21] | 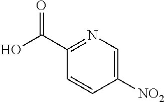 [SMILES: O=C(O)c1ccc([N+](=O)[O-])cn1] | 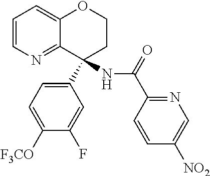 [SMILES: O=C(N[C@]1(c2ccc(OC(F)(F)F)c(F)c2)CCOc2cccnc21)c1ccc([N+](=O)[O-])cn1] | (S)-N-(4-(3- fluoro-4- (trifluoromethoxy) phenyl)- 3,4-dihydro- 2H-pyrano[3,2-b] pyridin-4-yl)-5- nitropicolinamide | 478.9 |
| 183 | 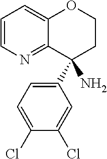 [SMILES: N[C@]1(c2ccc(Cl)c(Cl)c2)CCOc2cccnc21] | 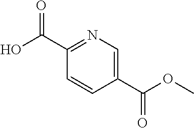 | 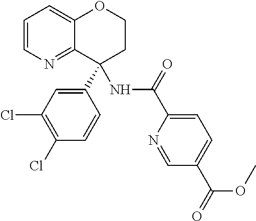 [SMILES: COC(=O)c1ccc(C(=O)N[C@]2(c3ccc(Cl)c(Cl)c3)CCOc3cccnc32)nc1] | (S)-methyl 6- ((4-(3,4- dichlorophenyl)- 3,4-dihydro-2H- pyrano[3,2-b] pyridin-4-yl) carbamoyl) nicotinate | 458.0 460.0 |
| 184 | 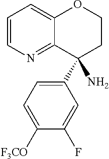 [SMILES: N[C@]1(c2ccc(OC(F)(F)F)c(F)c2)CCOc2cccnc21] | 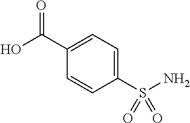 [SMILES: NS(=O)(=O)c1ccc(C(=O)O)cc1] | 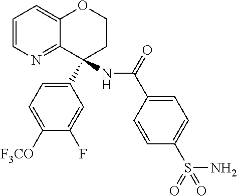 [SMILES: NS(=O)(=O)c1ccc(C(=O)N[C@]2(c3ccc(OC(F)(F)F)c(F)c3)CCOc3cccnc32)cc1] | (S)-N-(4-(3- fluoro-4- (trifluoromethoxy) phenyl)- 3,4-dihydro- 2H-pyrano[3,2-b] pyridin-4-yl)- sulfamoylbenzamide | 512 |
| 185 | 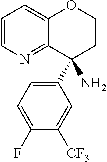 [SMILES: N[C@]1(c2ccc(F)c(C(F)(F)F)c2)CCOc2cccnc21] | 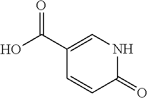 | 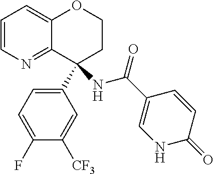 [SMILES: O=C(N[C@]1(c2ccc(F)c(C(F)(F)F)c2)CCOc2cccnc21)c1ccc(=O)[nH]c1] | (S)-N-(4-(4- fluoro-3- (trifluoromethyl) phenyl)- 3,4-dihydro- 2H-pyrano[3,2-b] pyridin-4-yl)- 6-oxo-1,6- dihydropyridine- 3-carboxamide | 434.1 |
| 186 | 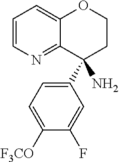 [SMILES: N[C@]1(c2ccc(OC(F)(F)F)c(F)c2)CCOc2cccnc21] | 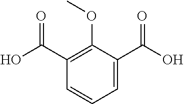 | 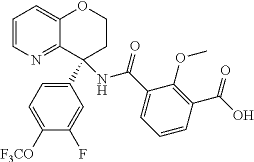 [SMILES: COc1c(C(=O)O)cccc1C(=O)N[C@]1(c2ccc(OC(F)(F)F)c(F)c2)CCOc2cccnc21] | (S)-3-((4-(3- fluoro-4- (trifluoromethoxy) phenyl)- 3,4-dihydro- 2H-pyrano[3,2-b] pyridin-4-yl) carbamoyl)-2- methoxybenzoic acid | 507.0 |
| 187 | 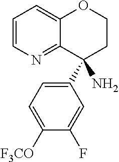 [SMILES: N[C@]1(c2ccc(OC(F)(F)F)c(F)c2)CCOc2cccnc21] | 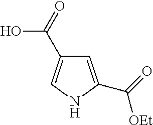 [SMILES: CCOC(=O)c1cc(C(=O)O)c[nH]1] | 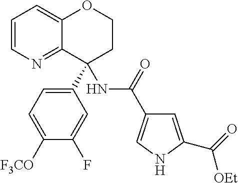 [SMILES: CCOC(=O)c1cc(C(=O)N[C@]2(c3ccc(OC(F)(F)F)c(F)c3)CCOc3cccnc32)c[nH]1] | (S)-ethyl 4- ((4-(3-fluoro-4- (trifluoromethoxy) phenyl)- 3,4-dihydro- 2H-pyrano[3,2-b] pyridin-4-yl) carbamoyl)- 1H-pyrrole- 2-carboxylate | 494.0 |
| 188 | 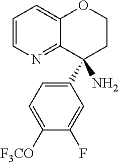 [SMILES: N[C@]1(c2ccc(OC(F)(F)F)c(F)c2)CCOc2cccnc21] | 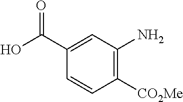 [SMILES: COC(=O)c1ccc(C(=O)O)cc1N] | 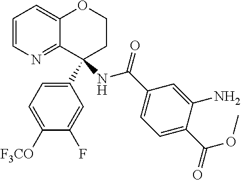 [SMILES: COC(=O)c1ccc(C(=O)N[C@]2(c3ccc(OC(F)(F)F)c(F)c3)CCOc3cccnc32)cc1N] | (S)-methyl 2- amino-4-((4- (3-fluoro-4- (trifluoromethoxy) phenyl)- 3,4-dihydro- 2H-pyrano[3,2-b] pyridin-4-yl) carbamoyl) benzoate | 506.0 |
| 189 | 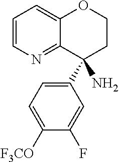 [SMILES: N[C@]1(c2ccc(OC(F)(F)F)c(F)c2)CCOc2cccnc21] | 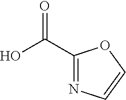 | 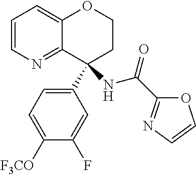 [SMILES: O=C(N[C@]1(c2ccc(OC(F)(F)F)c(F)c2)CCOc2cccnc21)c1ncco1] | (S)-N-(4-(3- fluoro-4- (trifluoromethoxy) phenyl)- 3,4-dihydro- 2H-pyrano[3,2-b] pyridin-4-yl) oxazole-2- carboxamide | 424.0 |
| 190 | 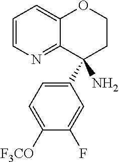 [SMILES: N[C@]1(c2ccc(OC(F)(F)F)c(F)c2)CCOc2cccnc21] | 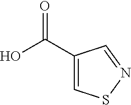 | 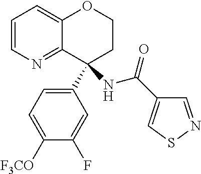 [SMILES: O=C(N[C@]1(c2ccc(OC(F)(F)F)c(F)c2)CCOc2cccnc21)c1cnsc1] | (S)-N-(4-(3- fluoro-4- (trifluoromethoxy) phenyl)- 3,4-dihydro- 2H-pyrano[3,2-b] pyridin-4-yl) isothiazole-4- carboxamide | 440.0 |
| 191 | 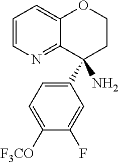 [SMILES: N[C@]1(c2ccc(OC(F)(F)F)c(F)c2)CCOc2cccnc21] | 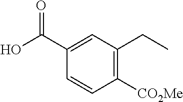 | 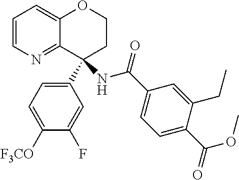 [SMILES: CCc1cc(C(=O)N[C@]2(c3ccc(OC(F)(F)F)c(F)c3)CCOc3cccnc32)ccc1C(=O)OC] | (S)-methyl 2- ethyl-4-((4- (3-fluoro-4- (trifluoromethoxy) phenyl)- 3,4-dihydro- 2H-pyrano[3,2-b] pyridin-4-yl) carbamoyl) benzoate | 519.0 |
| 192 | 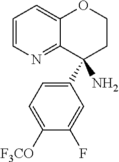 [SMILES: N[C@]1(c2ccc(OC(F)(F)F)c(F)c2)CCOc2cccnc21] | 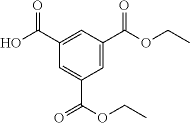 [SMILES: CCOC(=O)c1cc(C(=O)O)cc(C(=O)OCC)c1] | 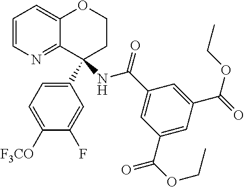 [SMILES: CCOC(=O)c1cc(C(=O)N[C@]2(c3ccc(OC(F)(F)F)c(F)c3)CCOc3cccnc32)cc(C(=O)OCC)c1] | (S)-diethyl 5- ((4-(3-fluoro-4- (trifluoromethoxy) phenyl)- 3,4-dihydro- 2H-pyrano[3,2-b] pyridin-4-yl) carbamoyl) isophthalate | 577.0 |
| 193 | 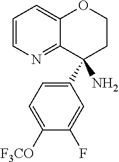 [SMILES: N[C@]1(c2ccc(OC(F)(F)F)c(F)c2)CCOc2cccnc21] | 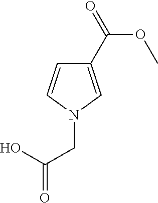 | 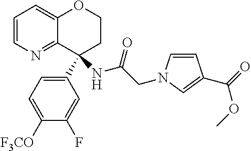 [SMILES: COC(=O)c1ccn(CC(=O)N[C@]2(c3ccc(OC(F)(F)F)c(F)c3)CCOc3cccnc32)c1] | (S)-melhyl 1- (2-((4-(3- fluoro-4- (trifluoromethoxy) phenyl)- 3,4-dihydro- 2H-pyrano[3,2-b] pyridin-4-yl) amino)-2- oxoethyl)-1H- pyrrole-3- carboxylate | 494.0 |
| Inter- mediate 46 | 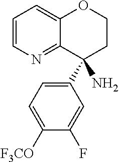 [SMILES: N[C@]1(c2ccc(OC(F)(F)F)c(F)c2)CCOc2cccnc21] | 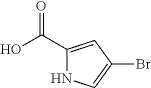 | 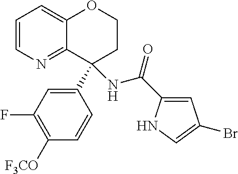 [SMILES: O=C(N[C@]1(c2ccc(OC(F)(F)F)c(F)c2)CCOc2cccnc21)c1cc(Br)c[nH]1] | (S)-4-bromo- N-(4-(3- fluoro-4- (trifluoromethoxy) phenyl)- 3,4-dihydro- 2H-pyrano[3,2-b] pyridin-4-yl)- 1H-pyrrole-2- carboxamide | 501.0 |
| 194 | 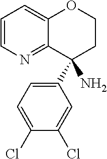 [SMILES: N[C@]1(c2ccc(Cl)c(Cl)c2)CCOc2cccnc21] | 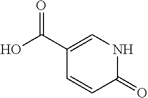 | 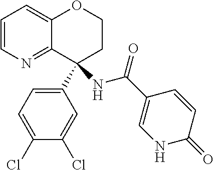 [SMILES: O=C(N[C@]1(c2ccc(Cl)c(Cl)c2)CCOc2cccnc21)c1ccc(=O)[nH]c1] | (S)-N-(4-(3,4- dichlorophenyl)- 3,4-dihydro-2H- pyrano[3,2-b] pyridin-4-yl)- 6-oxo-1,6- dihydropyridine- 3-carboxamide | 416.0 418.0 |
| 195 | 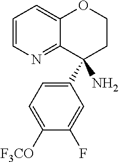 [SMILES: N[C@]1(c2ccc(OC(F)(F)F)c(F)c2)CCOc2cccnc21] | 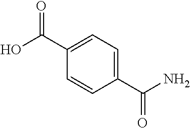 [SMILES: NC(=O)c1ccc(C(=O)O)cc1] | 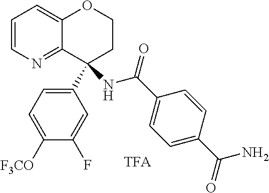 [SMILES: NC(=O)c1ccc(C(=O)N[C@]2(c3ccc(OC(F)(F)F)c(F)c3)CCOc3cccnc32)cc1] | (S)-N1-(4-(3- fluoro-4- (trifluoromethoxy) phenyl)- 3,4-dihydro- 2H-pyrano[3,2-b] pyridin-4-yl) terephthalamide 2,2,2- trifluoroacetate | 476.1 |
| 196 | 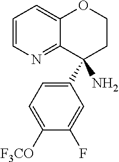 [SMILES: N[C@]1(c2ccc(OC(F)(F)F)c(F)c2)CCOc2cccnc21] | 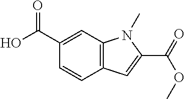 [SMILES: COC(=O)c1cc2ccc(C(=O)O)cc2n1C] | 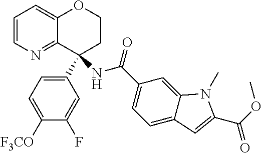 [SMILES: COC(=O)c1cc2ccc(C(=O)N[C@]3(c4ccc(OC(F)(F)F)c(F)c4)CCOc4cccnc43)cc2n1C] | (S)-methyl 6- ((4-(3-fluoro-4- (trifluoromethoxy) phenyl)- 3,4-dihydro- 2H-pyrano[3,2-b] pyridin-4-yl) carbamoyl)-1- methyl-1H- indole-2- carboxylate | 544.1 |
| 197 | 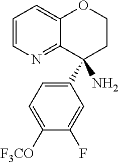 [SMILES: N[C@]1(c2ccc(OC(F)(F)F)c(F)c2)CCOc2cccnc21] | 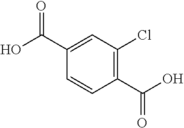 [SMILES: O=C(O)c1ccc(C(=O)O)c(Cl)c1] | 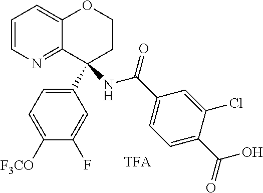 [SMILES: O=C(N[C@]1(c2ccc(OC(F)(F)F)c(F)c2)CCOc2cccnc21)c1ccc(C(=O)O)c(Cl)c1] | (S)-2-chloro- 4-((4-(3- fluoro-4- (trifluoromethoxy) phenyl)- 3,4-dihydro- 2H-pyrano[3,2-b] pyridin-4-yl) carbamoyl) benzoic acid 2,2,2- trifluoroacetate | 511.0 |
| 198 | 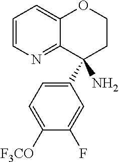 [SMILES: N[C@]1(c2ccc(OC(F)(F)F)c(F)c2)CCOc2cccnc21] | 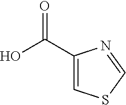 | 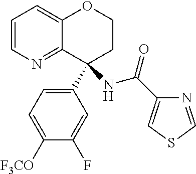 [SMILES: O=C(N[C@]1(c2ccc(OC(F)(F)F)c(F)c2)CCOc2cccnc21)c1cscn1] | (S)-N-(4-(3- fluoro-4- (trifluoromethoxy) phenyl)- 3,4-dihydro- 2H-pyrano[3,2-b] pyridin-4-yl) thiazole-4- carboxamide | 440.0 |
| 199 | 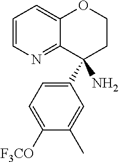 [SMILES: Cc1cc([C@@]2(N)CCOc3cccnc32)ccc1OC(F)(F)F] | 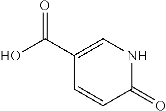 | 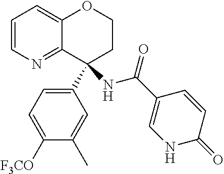 [SMILES: Cc1cc([C@@]2(NC(=O)c3ccc(=O)[nH]c3)CCOc3cccnc32)ccc1OC(F)(F)F] | (S)-N-(4-(3- methyl-4- (trifluoromethoxy) phenyl)- 3,4-dihydro- 2H-pyrano[3,2-b] pyridin-4-yl)- 6-oxo-1,6- dihydropyridine- 3-carboxamide | 446.1 |
| 200 | 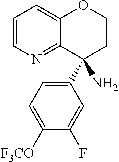 [SMILES: N[C@]1(c2ccc(OC(F)(F)F)c(F)c2)CCOc2cccnc21] | 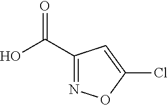 | 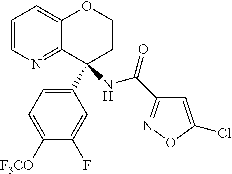 [SMILES: O=C(N[C@]1(c2ccc(OC(F)(F)F)c(F)c2)CCOc2cccnc21)c1cc(Cl)on1] | (S)-5-chloro- N-(4-(3- fluoro-4- (trifluoromethoxy) phenyl)- 3,4-dihydro- 2H-pyrano[3,2-b] pyridin-4-yl) isoxazole-3- carboxainide | 458.0 |
| 201 | 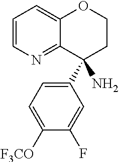 [SMILES: N[C@]1(c2ccc(OC(F)(F)F)c(F)c2)CCOc2cccnc21] | 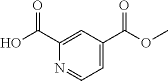 | 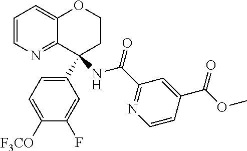 [SMILES: COC(=O)c1ccnc(C(=O)N[C@]2(c3ccc(OC(F)(F)F)c(F)c3)CCOc3cccnc32)c1] | (S)-methyl 2- ((4-(3-fluoro-4- (trifluoromethoxy) phenyl)- 3,4-dihydro- 2H-pyrano[3,2-b] pyridin-4-yl) carbamoyl) isonicotinate | 492.0 |
| 202 | 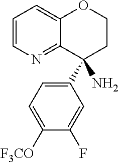 [SMILES: N[C@]1(c2ccc(OC(F)(F)F)c(F)c2)CCOc2cccnc21] | 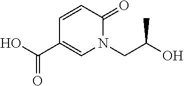 | 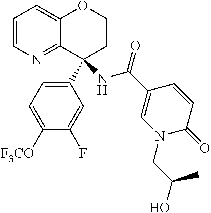 [SMILES: C[C@@H](O)Cn1cc(C(=O)N[C@]2(c3ccc(OC(F)(F)F)c(F)c3)CCOc3cccnc32)ccc1=O] | N-((S)-4-(3- fluoro-4- (trifluoromethoxy) phenyl)- 3,4-dihydro- 2H-pyrano[3,2-b] pyridin-4- yl)-1-((R)-2- hydroxypropyl)- 6-oxo-1,6- dihydropyridine- 3-carboxamide | 508.0 |
| 203 | 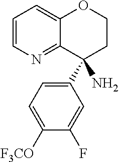 [SMILES: N[C@]1(c2ccc(OC(F)(F)F)c(F)c2)CCOc2cccnc21] | 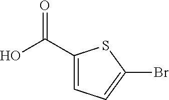 | 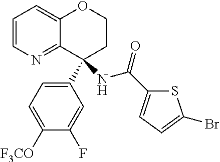 [SMILES: O=C(N[C@]1(c2ccc(OC(F)(F)F)c(F)c2)CCOc2cccnc21)c1ccc(Br)s1] | (S)-5-bromo- N-(4-(3- fluoro-4- (trifluoromethoxy) phenyl)- 3,4-dihydro- 2H-pyrano[3,2-b] pyridin-4-yl) thiophene- 2-carboxamide | 516.8 |
| 204 | 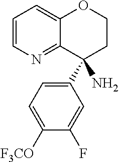 [SMILES: N[C@]1(c2ccc(OC(F)(F)F)c(F)c2)CCOc2cccnc21] | 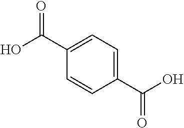 [SMILES: O=C(O)c1ccc(C(=O)O)cc1] | 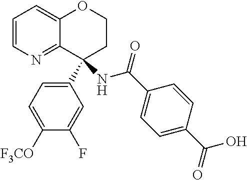 [SMILES: O=C(O)c1ccc(C(=O)N[C@]2(c3ccc(OC(F)(F)F)c(F)c3)CCOc3cccnc32)cc1] | (S)-4-((4-(3- fluoro-4- (trifluoromethoxy) phenyl)- 3,4-dihydro- 2H-pyrano[3,2-b] pyridin-4-yl) carbamoyl) benzoic acid | 475.1 |
| 205 | 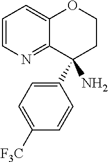 [SMILES: N[C@]1(c2ccc(C(F)(F)F)cc2)CCOc2cccnc21] | 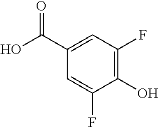 [SMILES: O=C(O)c1cc(F)c(O)c(F)c1] | 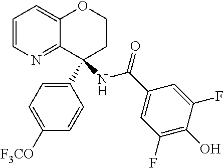 [SMILES: O=C(N[C@]1(c2ccc(OC(F)(F)F)cc2)CCOc2cccnc21)c1cc(F)c(O)c(F)c1] | (S)-3,5- difluoro-4- hydroxy-N-(4-(4- (trifluoromethyl) phenyl)- 3,4-dihydro- 2H-pyrano[3,2-b] pyridin-4-yl) benzamide | 451.1 |
| 206 | 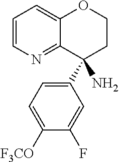 [SMILES: N[C@]1(c2ccc(OC(F)(F)F)c(F)c2)CCOc2cccnc21] | 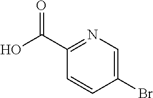 | 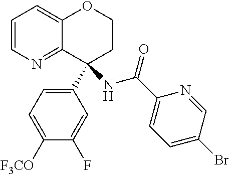 [SMILES: O=C(N[C@]1(c2ccc(OC(F)(F)F)c(F)c2)CCOc2cccnc21)c1ccc(Br)cn1] | (S)-5-bromo- N-(4-(3- fluoro-4- (trifluoromethoxy) phenyl)- 3,4-dihydro- 2H-pyrano[3,2-b] pyridin-4-yl) picolinamide | 511.9 |
| 207 | 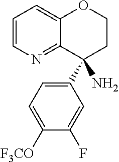 [SMILES: N[C@]1(c2ccc(OC(F)(F)F)c(F)c2)CCOc2cccnc21] | 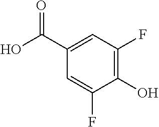 [SMILES: O=C(O)c1cc(F)c(O)c(F)c1] | 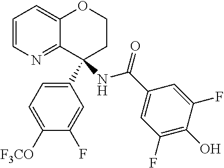 [SMILES: O=C(N[C@]1(c2ccc(OC(F)(F)F)c(F)c2)CCOc2cccnc21)c1cc(F)c(O)c(F)c1] | (S)-3,5- difluoro-N- (4-(3-fluoro-4- (trifluoromethoxy) phenyl)- 3,4-dihydro- 2H-pyrano[3,2-b] pyridin-4-yl)-4- hydroxybenzamide | 485.0 |
| 208 | 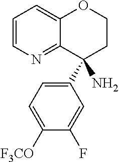 [SMILES: N[C@]1(c2ccc(OC(F)(F)F)c(F)c2)CCOc2cccnc21] | 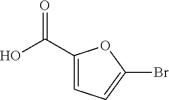 | 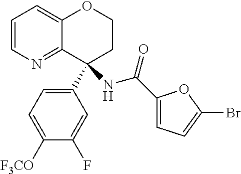 [SMILES: O=C(N[C@]1(c2ccc(OC(F)(F)F)c(F)c2)CCOc2cccnc21)c1ccc(Br)o1] | (S)-5-bromo- N-(4-(3- fluoro-4- (trifluoromethoxy) phenyl)- 3,4-dihydro- 2H-pyrano[3,2-b] pyridin-4- yl)furan-2- carboxamide | 502.9 |
| 209 | 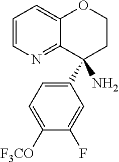 [SMILES: N[C@]1(c2ccc(OC(F)(F)F)c(F)c2)CCOc2cccnc21] | 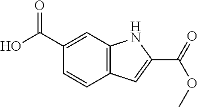 [SMILES: COC(=O)c1cc2ccc(C(=O)O)cc2[nH]1] | 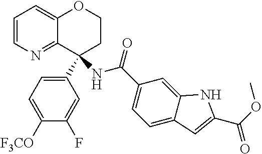 [SMILES: COC(=O)c1cc2ccc(C(=O)N[C@]3(c4ccc(OC(F)(F)F)c(F)c4)CCOc4cccnc43)cc2[nH]1] | (S)-methyl 6- ((4-(3-fluoro-4- (trifluoromethoxy) phenyl)- 3,4-dihydro- 2H-pyrano[3,2-b] pyridin-4- yl)carbamoyl)- 1H-indole-2- carboxylate | 530.0 |
| 210 | 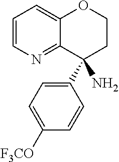 [SMILES: N[C@]1(c2ccc(OC(F)(F)F)cc2)CCOc2cccnc21] | 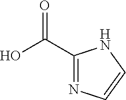 | 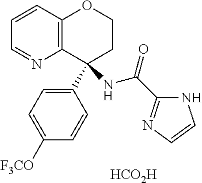 [SMILES: O=C(N[C@]1(c2ccc(OC(F)(F)F)cc2)CCOc2cccnc21)c1ncc[nH]1 ; "HCO2H"] | (S)-N-(4-(4- (trifluoromethoxy) phenyl)- 3,4-dihydro- 2H-pyrano[3,2-b] pyridin-4-yl)-1H- imidazole-2- carboxamide formate | 405.0 |
| 211 | 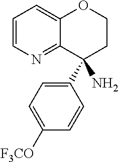 [SMILES: N[C@]1(c2ccc(OC(F)(F)F)cc2)CCOc2cccnc21] | 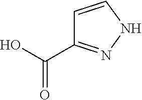 | 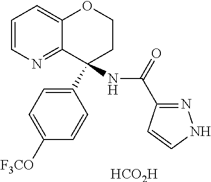 [SMILES: O=C(N[C@]1(c2ccc(OC(F)(F)F)cc2)CCOc2cccnc21)c1cc[nH]n1 ; "HCO2H"] | (S)-N-(4-(4- (trifluoromethoxy) phenyl)- 3,4-dihydro- 2H-pyrano[3,2-b] pyridin-4- yl)-1H- pyrazole-3- carboxamide formate | 405.0 |
| 212 | 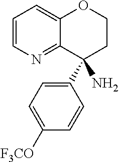 [SMILES: N[C@]1(c2ccc(OC(F)(F)F)cc2)CCOc2cccnc21] | 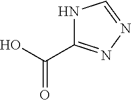 | 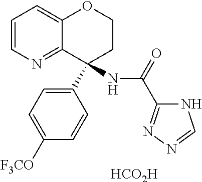 [SMILES: O=C(N[C@]1(c2ccc(OC(F)(F)F)cc2)CCOc2cccnc21)c1nnc[nH]1 ; "HCO2H"] | (S)-N-(4-(4- (trifluoromethoxy) phenyl)- 3,4-dihydro- 2H-pyrano[3,2-b] pyridin-4- yl)-4H-1,2,4- triazole-3- carboxamide formate | 406.0 |
| 213 | 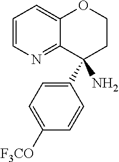 [SMILES: N[C@]1(c2ccc(OC(F)(F)F)cc2)CCOc2cccnc21] | 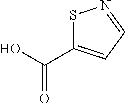 | 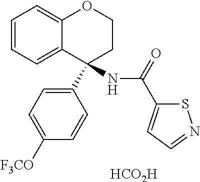 [SMILES: O=C(N[C@]1(c2ccc(OC(F)(F)F)cc2)CCOc2ccccc21)c1ccns1 ; "HCO2H"] | (S)-N-(4-(4- (trifluoromethoxy) phenyl)- 3,4-dihydro- 2H-pyrano[3,2-b] pyridin-4- yl)isothiazole-5- carboxamide formate | 422.0 |
| 214 | 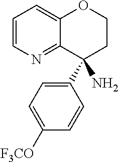 [SMILES: N[C@]1(c2ccc(OC(F)(F)F)cc2)CCOc2cccnc21] | 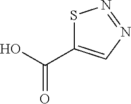 | 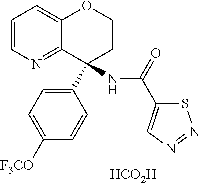 [SMILES: O=C(N[C@]1(c2ccc(OC(F)(F)F)cc2)CCOc2cccnc21)c1cnns1 ; "HCO2H"] | (S)-N-(4-(4- (trifluoromethoxy) phenyl)- 3,4-dihydro- 2H-pyrano[3,2-b] pyridin-4-yl)-1,2,3- thiadiazole-5- carboxamide formate | 423.0 |
| 215 | 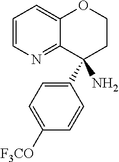 [SMILES: N[C@]1(c2ccc(OC(F)(F)F)cc2)CCOc2cccnc21] | 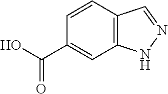 [SMILES: O=C(O)c1ccc2cn[nH]c2c1] | 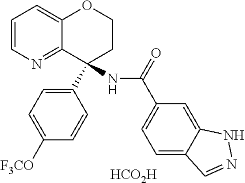 [SMILES: O=C(N[C@]1(c2ccc(OC(F)(F)F)cc2)CCOc2cccnc21)c1ccc2cn[nH]c2c1 ; "HCO2H"] | (S)-N-(4-(4- (trifluoromethoxy) phenyl)- 3,4-dihydro- 2H-pyrano[3,2-b] pyridin-4-yl)-1H- indazole-5- carboxamide formate | 455.0 |
| 216 | 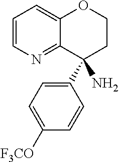 [SMILES: N[C@]1(c2ccc(OC(F)(F)F)cc2)CCOc2cccnc21] | 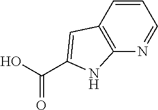 [SMILES: O=C(O)c1cc2cccnc2[nH]1] | 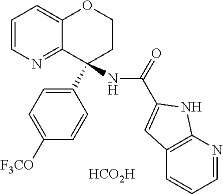 [SMILES: O=C(N[C@]1(c2ccc(OC(F)(F)F)cc2)CCOc2cccnc21)c1cc2cccnc2[nH]1 ; "HCO2H"] | (S)-N-(4-(4- (trifluoromethoxy) phenyl)- 3,4-dihydro- 2H-pyrano[3,2-b] pyridin-4-yl)-1H- pyrrolo[2,3-b] pyridine-2- carboxamide formate | 455.0 |
| 217 | 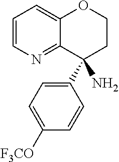 [SMILES: N[C@]1(c2ccc(OC(F)(F)F)cc2)CCOc2cccnc21] | 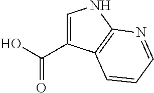 [SMILES: O=C(O)c1c[nH]c2ncccc12] | 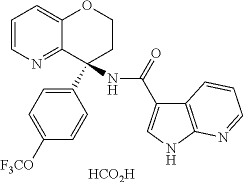 [SMILES: O=C(N[C@]1(c2ccc(OC(F)(F)F)cc2)CCOc2cccnc21)c1c[nH]c2ncccc12 ; "HCO2H"] | (S)-N-(4-(4- (trifluoromethoxy) phenyl)- 3,4-dihydro- 2H-pyrano[3,2-b] pyridin-4-yl)-1H- pyrrolo[2,3-b] pyridine-3- carboxamide fonnate | 455.0 |
| 218 | 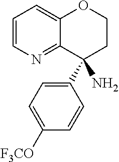 [SMILES: N[C@]1(c2ccc(OC(F)(F)F)cc2)CCOc2cccnc21] | 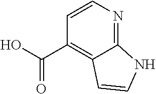 [SMILES: O=C(O)c1ccnc2[nH]ccc12] | 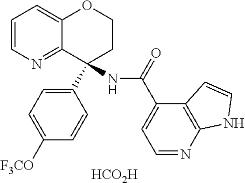 [SMILES: O=C(N[C@]1(c2ccc(OC(F)(F)F)cc2)CCOc2cccnc21)c1ccnc2[nH]ccc12 ; "HCO2H"] | (S)-N-(4-(4- (trifluoromethoxy) phenyl)- 3,4-dihydro- 2H-pyrano[3,2-b] pyridin-4-yl)-1H- pyrrolo[2,3-b] pyridine-4- carboxamide formate | 455.0 |
| 219 | 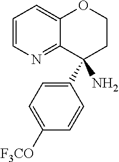 [SMILES: N[C@]1(c2ccc(OC(F)(F)F)cc2)CCOc2cccnc21] | 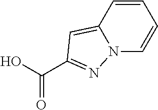 [SMILES: O=C(O)c1cc2ccccn2n1] | 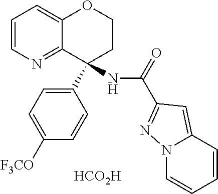 [SMILES: O=C(N[C@]1(c2ccc(OC(F)(F)F)cc2)CCOc2cccnc21)c1cc2ccccn2n1 ; "HCO2H"] | (S)-N-(4-(4- (trifluoromethoxy) phenyl)- 3,4-dihydro- 2H-pyrano[3,2-b] pyridin-4-yl) pyrazolo[1,5-a] pyridine-2- carboxainide formate | 455.0 |
| 220 | 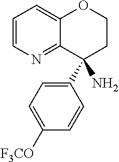 [SMILES: N[C@]1(c2ccc(OC(F)(F)F)cc2)CCOc2cccnc21] | 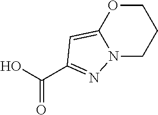 [SMILES: O=C(O)c1cc2n(n1)CCCO2] | 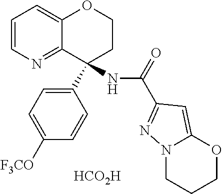 [SMILES: O=C(N[C@]1(c2ccc(OC(F)(F)F)cc2)CCOc2cccnc21)c1cc2n(n1)CCCO2 ; "HCO2H"] | (S)-N-(4-(4- (trifluoromethoxy) phenyl)- 3,4-dihydro- 2H-pyrano[3,2-b] pyridin-4-yl)-6,7- dihydro-5H- pyrazolo[5,1-b] [1,3]oxazine-2- carboxamide formate | 461.0 |
| 221 | 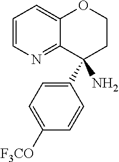 [SMILES: N[C@]1(c2ccc(OC(F)(F)F)cc2)CCOc2cccnc21] | 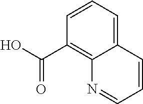 [SMILES: O=C(O)c1cccc2cccnc12] | 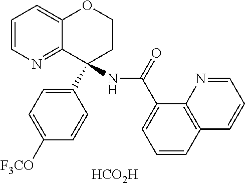 [SMILES: O=C(N[C@]1(c2ccc(OC(F)(F)F)cc2)CCOc2cccnc21)c1cccc2cccnc12 ; "HCO2H"] | (S)-N-(4-(4- (trifluoromethoxy) phenyl)- 3,4-dihydro- 2H-pyrano[3,2-b] pyridin-4- yl)quinoline- 8-carboxamide formate | 466.0 |
| 222 | 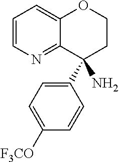 [SMILES: N[C@]1(c2ccc(OC(F)(F)F)cc2)CCOc2cccnc21] | 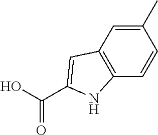 | 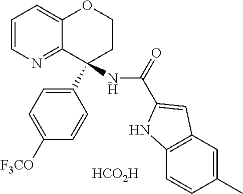 [SMILES: Cc1ccc2[nH]c(C(=O)N[C@]3(c4ccc(OC(F)(F)F)cc4)CCOc4cccnc43)cc2c1 ; "HCO2H"] | (S)-5-methyl- N-(4-(4- (trifluoromethoxy) phenyl)- 3,4-dihydro- 2H-pyrano[3,2-b] pyridin-4-yl)-1H- indole-2- carboxamide formate | 468.0 |
| 223 | 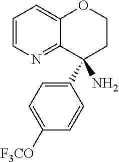 [SMILES: N[C@]1(c2ccc(OC(F)(F)F)cc2)CCOc2cccnc21] | 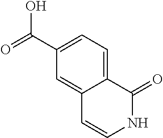 [SMILES: O=C(O)c1ccc2c(=O)[nH]ccc2c1] | 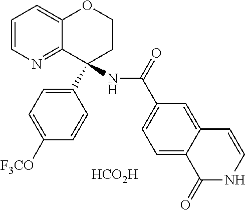 [SMILES: O=C(N[C@]1(c2ccc(OC(F)(F)F)cc2)CCOc2cccnc21)c1ccc2c(=O)[nH]ccc2c1 ; "HCO2H"] | (S)-1-oxo-N- (4-(4- (trifluoromethoxy) phenyl)- 3,4-dihydro- 2H-pyrano[3,2-b] pyridin-4-yl)-1,2- dihydroisoquinoline- 6-carboxamide formate | 482.0 |
| 224 | 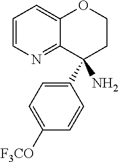 [SMILES: N[C@]1(c2ccc(OC(F)(F)F)cc2)CCOc2cccnc21] | 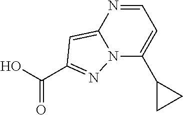 [SMILES: O=C(O)c1cc2nccc(C3CC3)n2n1] | 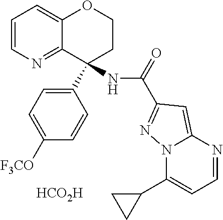 [SMILES: O=C(N[C@]1(c2ccc(OC(F)(F)F)cc2)CCOc2cccnc21)c1cc2nccc(C3CC3)n2n1 ; "HCO2H"] | (S)-7- cyclopropyl- N-(4-(4- (trifluoromethoxy) phenyl)- 3,4-dihydro- 2H-pyrano[3,2-b] pyridin-4-yl) pyrazolo[1,5-a] pyrimidine-2- carboxamide formate | 496.0 |
| 225 | 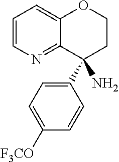 [SMILES: N[C@]1(c2ccc(OC(F)(F)F)cc2)CCOc2cccnc21] | 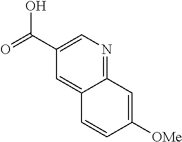 [SMILES: COc1ccc2cc(C(=O)O)cnc2c1] | 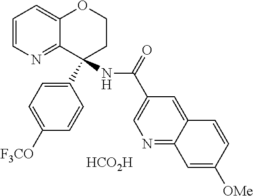 [SMILES: COc1ccc2cc(C(=O)N[C@]3(c4ccc(OC(F)(F)F)cc4)CCOc4cccnc43)cnc2c1 ; "HCO2H"] | (S)-7- methoxy-N- (4-(4- (trifluoromethoxy) phenyl)- 3,4-dihydro- 2H-pyrano[3,2-b] pyridin-4- yl)quinoline-3- carboxamide formate | 496.0 |
| 226 | 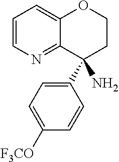 [SMILES: N[C@]1(c2ccc(OC(F)(F)F)cc2)CCOc2cccnc21] | 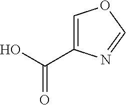 | 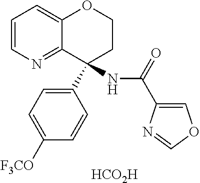 [SMILES: O=C(N[C@]1(c2ccc(OC(F)(F)F)cc2)CCOc2cccnc21)c1cocn1 ; "HCO2H"] | (S)-N-(4-(4- (trifluoromethoxy) phenyl)- 3,4-dihydro- 2H-pyrano[3,2-b] pyridin-4- yl)oxazole-4- carboxamide formate | 406.0 |
| 227 | 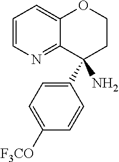 [SMILES: N[C@]1(c2ccc(OC(F)(F)F)cc2)CCOc2cccnc21] | 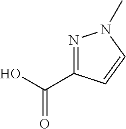 | 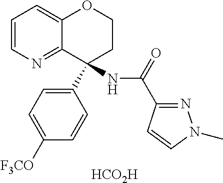 [SMILES: Cn1ccc(C(=O)N[C@]2(c3ccc(OC(F)(F)F)cc3)CCOc3cccnc32)n1 ; "HCO2H"] | (S)-1-methyl- N-(4-(4- (trifluoromethoxy) phenyl)- 3,4-dihydro- 2H-pyrano[3,2-b] pyridin-4- yl)-1H- pyrazole-3- carboxamide formate | 419.0 |
| 228 | 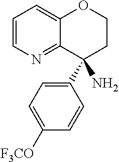 [SMILES: N[C@]1(c2ccc(OC(F)(F)F)cc2)CCOc2cccnc21] | 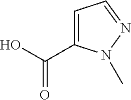 | 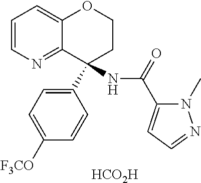 [SMILES: Cn1nccc1C(=O)N[C@]1(c2ccc(OC(F)(F)F)cc2)CCOc2cccnc21 ; "HCO2H"] | (S)-1-methyl- N-(4-(4- (trifluorometh oxy)phenyl)- 3,4-dihydro- 2H- pyrano[3,2- b]pyridin-4- yl)-lH- pyrazole-5- carboxamide formate_ | 419.0 |
| 229 | 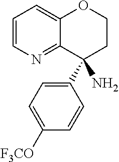 [SMILES: N[C@]1(c2ccc(OC(F)(F)F)cc2)CCOc2cccnc21] | 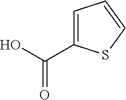 | 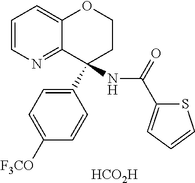 [SMILES: O=C(N[C@]1(c2ccc(OC(F)(F)F)cc2)CCOc2cccnc21)c1cccs1 ; "HCO2H"] | (S)-N-(4-(4- (trifluoromethoxy) phenyl)- 3,4-dihydro- 2H-pyrano[3,2-b] pyridin-4- yl)thiophene- 2-carboxamide fomiate | 421.0 |
| 230 | 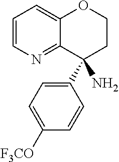 [SMILES: N[C@]1(c2ccc(OC(F)(F)F)cc2)CCOc2cccnc21] | 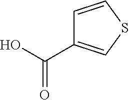 | 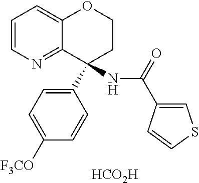 [SMILES: O=C(N[C@]1(c2ccc(OC(F)(F)F)cc2)CCOc2cccnc21)c1ccsc1 ; "HCO2H"] | (S)-N-(4-(4- (trifluoromethoxy) phenyl)- 3,4-dihydro- 2H-pyrano[3,2-b] pyridin-4- yl)thiophene- 3-carboxamide formate | 421.0 |
| 231 | 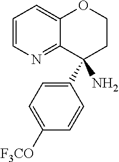 [SMILES: N[C@]1(c2ccc(OC(F)(F)F)cc2)CCOc2cccnc21] | 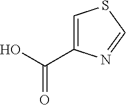 | 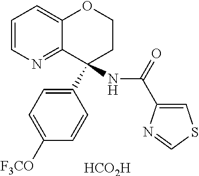 [SMILES: O=C(N[C@]1(c2ccc(OC(F)(F)F)cc2)CCOc2cccnc21)c1cscn1 ; "HCO2H"] | (S)-N-(4-(4- (trifluoromethoxy) phenyl)- 3,4-dihydro- 2H-pyrano[3,2-b] pyridin-4- yl)thiazole-4- carboxamide formate | 422.0 |
| 232 | 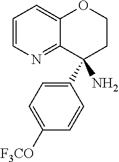 [SMILES: N[C@]1(c2ccc(OC(F)(F)F)cc2)CCOc2cccnc21] | 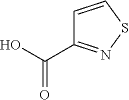 | 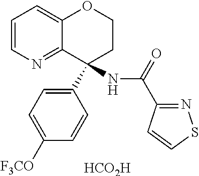 [SMILES: O=C(N[C@]1(c2ccc(OC(F)(F)F)cc2)CCOc2cccnc21)c1ccsn1 ; "HCO2H"] | (S)-N-(4-(4- (trifluoromethoxy) phenyl)- 3,4-dihydro- 2H-pyrano[3,2-b] pyridin-4- yl)isothiazole-3- carboxamide formate | 422.0 |
| 233 | 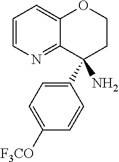 [SMILES: N[C@]1(c2ccc(OC(F)(F)F)cc2)CCOc2cccnc21] | 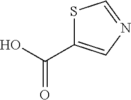 | 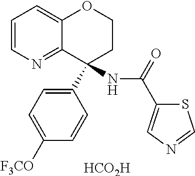 [SMILES: O=C(N[C@]1(c2ccc(OC(F)(F)F)cc2)CCOc2cccnc21)c1cncs1 ; "HCO2H"] | (S)-N-(4-(4- (trifluoromethoxy) phenyl)- 3,4-dihydro- 2H-pyrano[3,2-b] pyridin-4- yl)thiazole-5- carboxamide formate | 422.0 |
| 234 | 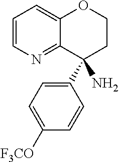 [SMILES: N[C@]1(c2ccc(OC(F)(F)F)cc2)CCOc2cccnc21] | 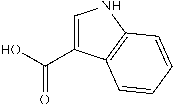 [SMILES: O=C(O)c1c[nH]c2ccccc12] | 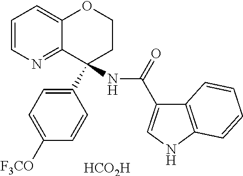 [SMILES: O=C(N[C@]1(c2ccc(OC(F)(F)F)cc2)CCOc2cccnc21)c1c[nH]c2ccccc12 ; "HCO2H"] | (S)-N-(4-(4- (trifluoromethoxy) phenyl)- 3,4-dihydro- 2H-pyrano[3,2-b] pyridin-4- yl)-1H- indole-3- carboxamide foraiate | 454.0 |
| 235 | 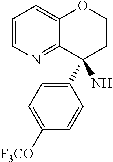 [SMILES: N[C@]1(c2ccc(OC(F)(F)F)cc2)CCOc2cccnc21] | 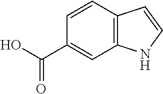 [SMILES: O=C(O)c1ccc2cc[nH]c2c1] | 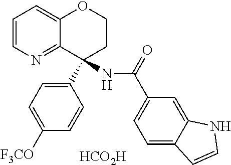 [SMILES: O=C(N[C@]1(c2ccc(OC(F)(F)F)cc2)CCOc2cccnc21)c1ccc2cc[nH]c2c1 ; "HCO2H"] | (S)-N-(4-(4- (trifluoromethoxy) phenyl)- 3,4-dihydro- 2H-pyrano[3,2-b] pyridin-4- yl)-1H- indole-6- carboxamide formate | 454.0 |
| 236 | 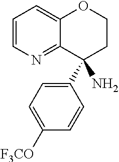 [SMILES: N[C@]1(c2ccc(OC(F)(F)F)cc2)CCOc2cccnc21] | 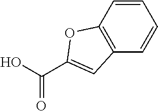 [SMILES: O=C(O)c1cc2ccccc2o1] | 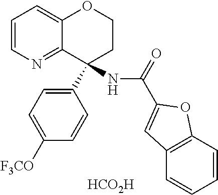 [SMILES: O=C(N[C@]1(c2ccc(OC(F)(F)F)cc2)CCOc2cccnc21)c1cc2ccccc2o1 ; "HCO2H"] | (S)-N-(4-(4- (trifluoromethoxy) phenyl)- 3,4-dihydro- 2H-pyrano[3,2-b] pyridin-4- yl)benzofuran-2- carboxamide formate | 455.0 |
| 237 | 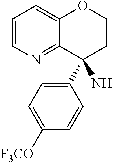 [SMILES: N[C@]1(c2ccc(OC(F)(F)F)cc2)CCOc2cccnc21] | 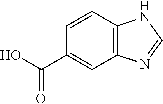 [SMILES: O=C(O)c1ccc2[nH]cnc2c1] | 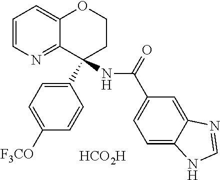 [SMILES: O=C(N[C@]1(c2ccc(OC(F)(F)F)cc2)CCOc2cccnc21)c1ccc2[nH]cnc2c1 ; "HCO2H"] | (S)-N-(4-(4- (trifluoromethoxy) phenyl)- 3,4-dihydro- 2H-pyrano[3,2-b] pyridin-4- yl)1lH- benzo[d]imidazole- 5-carboxamide formate | 455.0 |
| 238 | 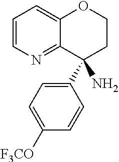 [SMILES: N[C@]1(c2ccc(OC(F)(F)F)cc2)CCOc2cccnc21] | 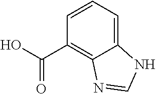 [SMILES: O=C(O)c1cccc2[nH]cnc12] | 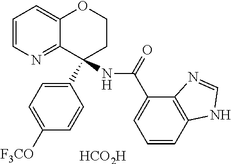 [SMILES: O=C(N[C@]1(c2ccc(OC(F)(F)F)cc2)CCOc2cccnc21)c1cccc2[nH]cnc12 ; "HCO2H"] | (S)-N-(4-(4- (trifluoromethoxy) phenyl)- 3,4-dihydro- 2H-pyrano[3,2-b] pyridin-4- yl)-1H- benzo[d]imidazole- 4-carboxamide formate | 455.0 |
| 239 | 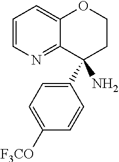 [SMILES: N[C@]1(c2ccc(OC(F)(F)F)cc2)CCOc2cccnc21] | 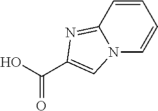 [SMILES: O=C(O)c1cn2ccccc2n1] | 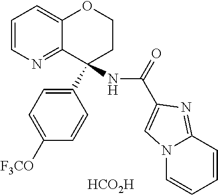 [SMILES: O=C(N[C@]1(c2ccc(OC(F)(F)F)cc2)CCOc2cccnc21)c1cn2ccccc2n1 ; "HCO2H"] | (S)-N-(4-(4- (trifluoromethoxy) phenyl)- 3,4-dihydro- 2H-pyrano[3,2-b] pyridin-4- yl)imidazo[1,2-a] pyridine-2- carboxamide formate | 455.0 |
| 240 | 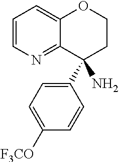 [SMILES: N[C@]1(c2ccc(OC(F)(F)F)cc2)CCOc2cccnc21] | 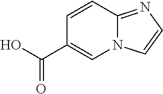 [SMILES: O=C(O)c1ccc2nccn2c1] | 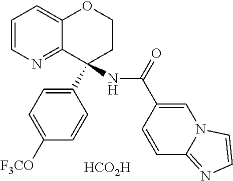 [SMILES: O=C(N[C@]1(c2ccc(OC(F)(F)F)cc2)CCOc2cccnc21)c1ccc2nccn2c1 ; "HCO2H"] | (S)-N-(4-(4- (trifluoromethoxy) phenyl)- 3,4-dihydro- 2H-pyrano[3,2-b] pyridin-4- yl)imidazo[1,2-a] pyridine-6- carboxamide formate | 455.0 |
| 241 | 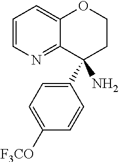 [SMILES: N[C@]1(c2ccc(OC(F)(F)F)cc2)CCOc2cccnc21] | 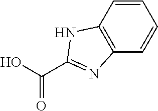 [SMILES: O=C(O)c1nc2ccccc2[nH]1] | 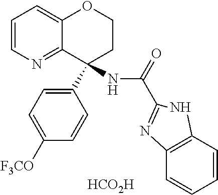 [SMILES: O=C(N[C@]1(c2ccc(OC(F)(F)F)cc2)CCOc2cccnc21)c1nc2ccccc2[nH]1 ; "HCO2H"] | (S)-N-(4-(4- (trifluoromethoxy) phenyl)- 3,4-dihydro- 2H-pyrano[3,2-b] pyridin-4- yl)-1H- benzo[d]imidazole- 2-carboxamide formate | 455.0 |
| 242 | 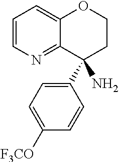 [SMILES: N[C@]1(c2ccc(OC(F)(F)F)cc2)CCOc2cccnc21] | 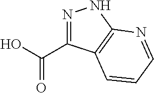 [SMILES: O=C(O)c1n[nH]c2ncccc12] | 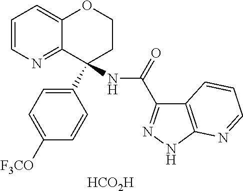 [SMILES: O=C(N[C@]1(c2ccc(OC(F)(F)F)cc2)CCOc2cccnc21)c1n[nH]c2ncccc12 ; "HCO2H"] | (S)-N-(4-(4- (trifluoromethoxy) phenyl)- 3,4-dihydro- 2H-pyrano[3,2-b] pyridin-4- yl)-1H- pyrazolo[3,4-b] pyridine-3- carboxamide formate | 456.0 |
| 243 | 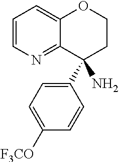 [SMILES: N[C@]1(c2ccc(OC(F)(F)F)cc2)CCOc2cccnc21] | 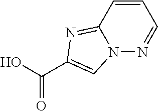 [SMILES: O=C(O)c1cn2ncccc2n1] | 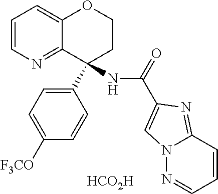 [SMILES: O=C(N[C@]1(c2ccc(OC(F)(F)F)cc2)CCOc2cccnc21)c1cn2ncccc2n1 ; "HCO2H"] | (S)-N-(4-(4- (trifluoromethoxy) phenyl)- 3,4-dihydro- 2H-pyrano[3,2-b] pyridin-4- yl)imidazo[1,2-b] pyridazine-2- carboxamide formate | 456.0 |
| 244 | 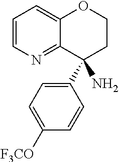 [SMILES: N[C@]1(c2ccc(OC(F)(F)F)cc2)CCOc2cccnc21] | 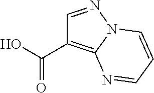 [SMILES: O=C(O)c1cnn2cccnc12] | 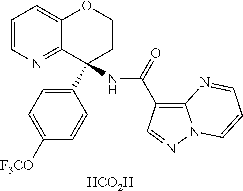 [SMILES: O=C(N[C@]1(c2ccc(OC(F)(F)F)cc2)CCOc2cccnc21)c1cnn2cccnc12 ; "HCO2H"] | (S)-N-(4-(4- (trifluoromethoxy) phenyl)- 3,4-dihydro- 2H-pyrano[3,2-b] pyridin-4- yl)pyrazolo[1,5-a] pyrimidine-3- carboxamide formate | 456.0 |
| 245 | 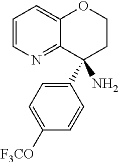 [SMILES: N[C@]1(c2ccc(OC(F)(F)F)cc2)CCOc2cccnc21] | 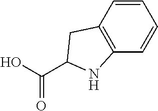 [SMILES: O=C(O)C1Cc2ccccc2N1] | 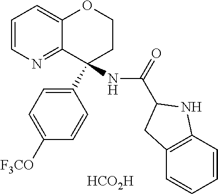 [SMILES: O=C(N[C@]1(c2ccc(OC(F)(F)F)cc2)CCOc2cccnc21)C1Cc2ccccc2N1 ; "HCO2H"] | N-((S)-4-(4- (trifluoromethoxy) phenyl)- 3,4-dihydro- 2H-pyrano[3,2-b] pyridin-4- yl)indoline-2- carboxamide formate | 456.0 |
| 246 | 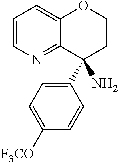 [SMILES: N[C@]1(c2ccc(OC(F)(F)F)cc2)CCOc2cccnc21] | 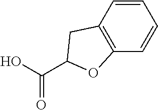 [SMILES: O=C(O)C1Cc2ccccc2O1] | 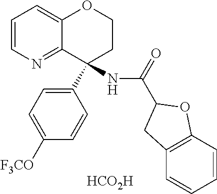 [SMILES: O=C(N[C@]1(c2ccc(OC(F)(F)F)cc2)CCOc2cccnc21)C1Cc2ccccc2O1 ; "HCO2H"] | N-((S)-4-(4- (trifluoromethoxy) phenyl)- 3,4-dihydro- 2H-pyrano[3,2-b] pyridin-4- yl)-2,3- dihydrobenzo furan-2- carboxamide formate | 457.0 |
| 247 | 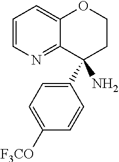 [SMILES: N[C@]1(c2ccc(OC(F)(F)F)cc2)CCOc2cccnc21] | 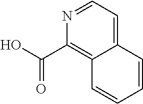 [SMILES: O=C(O)c1nccc2ccccc12] | 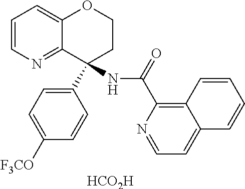 [SMILES: O=C(N[C@]1(c2ccc(OC(F)(F)F)cc2)CCOc2cccnc21)c1nccc2ccccc12 ; "HCO2H"] | (S)-N-(4-(4- (trifluoromethoxy) phenyl)- 3,4-dihydro- 2H-pyrano[3,2-b] pyridin-4- yl)isoquinoline-1- carboxamide formate | 466.0 |
| 248 | 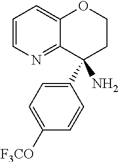 [SMILES: N[C@]1(c2ccc(OC(F)(F)F)cc2)CCOc2cccnc21] | 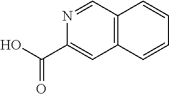 [SMILES: O=C(O)c1cc2ccccc2cn1] | 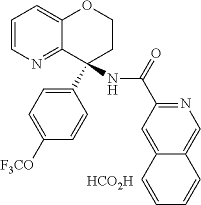 [SMILES: O=C(N[C@]1(c2ccc(OC(F)(F)F)cc2)CCOc2cccnc21)c1cc2ccccc2cn1 ; "HCO2H"] | (S)-N-(4-(4- (trifluoromethoxy) phenyl)- 3,4-dihydro- 2H-pyrano[3,2-b] pyridin-4- yl)isoquinoline-3- carboxamide formate | 466.0 |
| 249 | 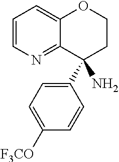 [SMILES: N[C@]1(c2ccc(OC(F)(F)F)cc2)CCOc2cccnc21] | 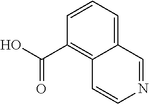 [SMILES: O=C(O)c1cccc2cnccc12] | 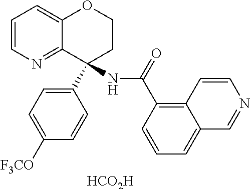 [SMILES: O=C(N[C@]1(c2ccc(OC(F)(F)F)cc2)CCOc2cccnc21)c1cccc2cnccc12 ; "HCO2H"] | (S)-N-(4-(4- (trifluoromethoxy) phenyl)- 3,4-dihydro- 2H-pyrano[3,2-b] pyridin-4- yl)isoquinoline- 5-carboxamide formate | 466.0 |
| 250 | 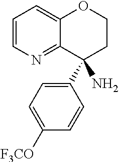 [SMILES: N[C@]1(c2ccc(OC(F)(F)F)cc2)CCOc2cccnc21] | 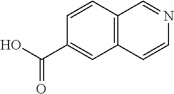 [SMILES: O=C(O)c1ccc2cnccc2c1] | 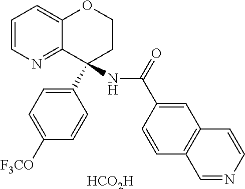 [SMILES: O=C(N[C@]1(c2ccc(OC(F)(F)F)cc2)CCOc2cccnc21)c1ccc2cnccc2c1 ; "HCO2H"] | (S)-N-(4-(4- (trifluoromethoxy) phenyl)- 3,4-dihydro- 2H-pyrano[3,2-b] pyridin-4- yl)isoquinoline- 6-carboxamide formate | 466.0 |
| 251 | 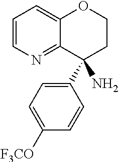 [SMILES: N[C@]1(c2ccc(OC(F)(F)F)cc2)CCOc2cccnc21] | 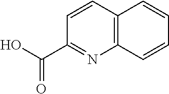 [SMILES: O=C(O)c1ccc2ccccc2n1] | 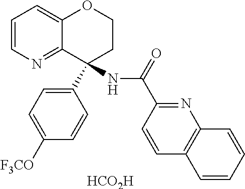 [SMILES: O=C(N[C@]1(c2ccc(OC(F)(F)F)cc2)CCOc2cccnc21)c1ccc2ccccc2n1 ; "HCO2H"] | (S)-N-(4-(4- (trifluoromethoxy) phenyl)- 3,4-dihydro- 2H-pyrano[3,2-b] pyridin-4- yl)quinoline- 2-carboxamide formate | 466.0 |
| 252 | 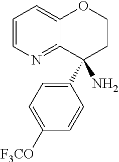 [SMILES: N[C@]1(c2ccc(OC(F)(F)F)cc2)CCOc2cccnc21] | 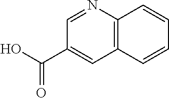 [SMILES: O=C(O)c1cnc2ccccc2c1] | 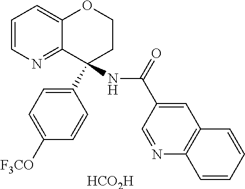 [SMILES: O=C(N[C@]1(c2ccc(OC(F)(F)F)cc2)CCOc2cccnc21)c1cnc2ccccc2c1 ; "HCO2H"] | (S)-N-(4-(4- (trifluoromethoxy) phenyl)- 3,4-dihydro- 2H-pyrano[3,2-b] pyridin-4- yl)quinoline- 3-carboxamide formate | 466.0 |
| 253 | 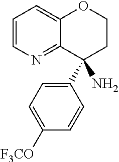 [SMILES: N[C@]1(c2ccc(OC(F)(F)F)cc2)CCOc2cccnc21] | 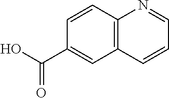 [SMILES: O=C(O)c1ccc2ncccc2c1] | 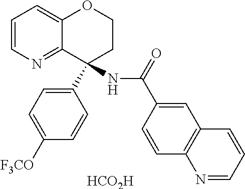 [SMILES: O=C(N[C@]1(c2ccc(OC(F)(F)F)cc2)CCOc2cccnc21)c1ccc2ncccc2c1 ; "HCO2H"] | (S)-N-(4-(4- (trifluoromethoxy) phenyl)- 3,4-dihydro- 2H-pyrano[3,2-b] pyridin-4- yl)quinoline- 6-carboxamide formate | 466.0 |
| 254 | 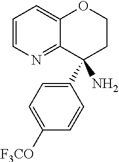 [SMILES: N[C@]1(c2ccc(OC(F)(F)F)cc2)CCOc2cccnc21] | 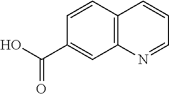 [SMILES: O=C(O)c1ccc2cccnc2c1] | 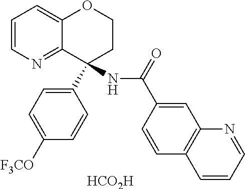 [SMILES: O=C(N[C@]1(c2ccc(OC(F)(F)F)cc2)CCOc2cccnc21)c1ccc2cccnc2c1 ; "HCO2H"] | (S)-N-(4-(4- (trifluoromethoxy) phenyl)- 3,4-dihydro- 2H-pyrano[3,2-b] pyridin-4- yl)quinoline- 7-carboxamide fonnate | 466.0 |
| 255 | 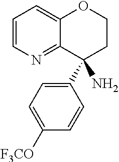 [SMILES: N[C@]1(c2ccc(OC(F)(F)F)cc2)CCOc2cccnc21] | 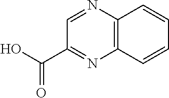 [SMILES: O=C(O)c1cnc2ccccc2n1] | 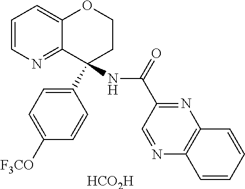 [SMILES: O=C(N[C@]1(c2ccc(OC(F)(F)F)cc2)CCOc2cccnc21)c1cnc2ccccc2n1 ; "HCO2H"] | (S)-N-(4-(4- (trifluoromethoxy) phenyl)- 3,4-dihydro- 2H-pyrano[3,2-b] pyridin-4- yl)quinoxaline- 2-carboxamide formate | 467.0 |
| 256 | 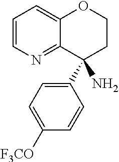 [SMILES: N[C@]1(c2ccc(OC(F)(F)F)cc2)CCOc2cccnc21] | 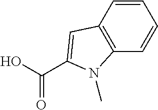 | 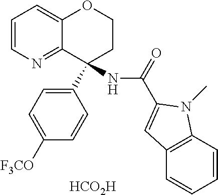 [SMILES: Cn1c(C(=O)N[C@]2(c3ccc(OC(F)(F)F)cc3)CCOc3cccnc32)cc2ccccc21 ; "HCO2H"] | (S)-1-methyl- N-(4-(4- (trifluoromethoxy) phenyl)- 3,4-dihydro- 2H-pyrano[3,2-b] pyridin-4- yl)-1H- indole-2- carboxamide formate | 468.2 |
| 257 | 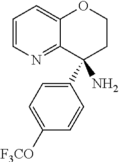 [SMILES: N[C@]1(c2ccc(OC(F)(F)F)cc2)CCOc2cccnc21] | 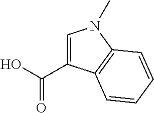 | 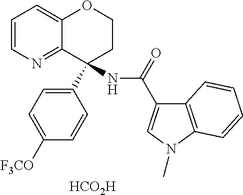 [SMILES: Cn1cc(C(=O)N[C@]2(c3ccc(OC(F)(F)F)cc3)CCOc3cccnc32)c2ccccc21 ; "HCO2H"] | (S)-1-methyl- N-(4-(4- (trifluoromethoxy) phenyl)- 3,4-dihydro- 2H-pyrano[3,2-b] pyridin-4- yl)-1H- indole-3- carboxamide formate | 468.2 |
| 258 | 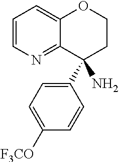 [SMILES: N[C@]1(c2ccc(OC(F)(F)F)cc2)CCOc2cccnc21] | 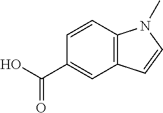 | 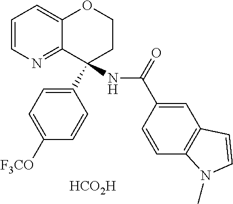 [SMILES: Cn1ccc2cc(C(=O)N[C@]3(c4ccc(OC(F)(F)F)cc4)CCOc4cccnc43)ccc21 ; "HCO2H"] | (S)-1-methyl- N-(4-(4- (trifluoromethoxy) phenyl)- 3,4-dihydro- 2H-pyrano[3,2-b] pyridin-4- yl)-1H- indole-5- carboxamide formate | 468.2 |
| 259 | 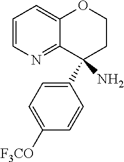 [SMILES: N[C@]1(c2ccc(OC(F)(F)F)cc2)CCOc2cccnc21] | 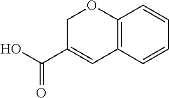 [SMILES: O=C(O)C1=Cc2ccccc2OC1] | 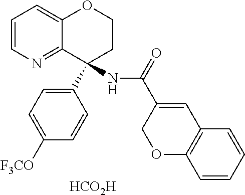 [SMILES: O=C(N[C@]1(c2ccc(OC(F)(F)F)cc2)CCOc2cccnc21)C1=Cc2ccccc2OC1 ; "HCO2H"] | (S)-N-(4-(4- (trifluoromethoxy) phenyl)- 3,4-dihydro- 2H-pyrano[3,2-b] pyridin-4- yl)-2H- chromene-3- carboxamide formate | 469.0 |
| 260 | 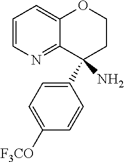 [SMILES: N[C@]1(c2ccc(OC(F)(F)F)cc2)CCOc2cccnc21] | 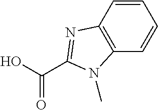 | 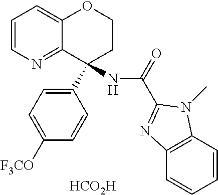 [SMILES: Cn1c(C(=O)N[C@]2(c3ccc(OC(F)(F)F)cc3)CCOc3cccnc32)nc2ccccc21 ; "HCO2H"] | (S)-1-methyl- N-(4-(4- (trifluoromethoxy) phenyl)- 3,4-dihydro- 2H-pyrano[3,2-b] pyridin-4- yl)-1H- benzo[d]imidazole- 2-carboxamide formate | 469.0 |
| 261 | 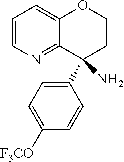 [SMILES: N[C@]1(c2ccc(OC(F)(F)F)cc2)CCOc2cccnc21] | 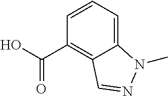 | 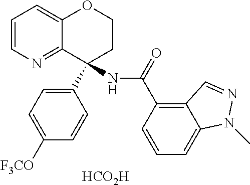 [SMILES: Cn1ncc2c(C(=O)N[C@]3(c4ccc(OC(F)(F)F)cc4)CCOc4cccnc43)cccc21 ; "HCO2H"] | (S)-1-methyl- N-(4-(4- (trifluoromethoxy) phenyl)- 3,4-dihydro- 2H-pyrano[3,2-b] pyridin-4- yl)-1H- indazole-4- carboxamide formate | 469.2 |
| 262 | 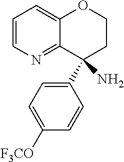 [SMILES: N[C@]1(c2ccc(OC(F)(F)F)cc2)CCOc2cccnc21] | 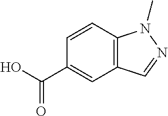 | 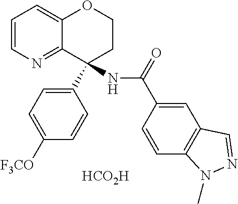 [SMILES: Cn1ncc2cc(C(=O)N[C@]3(c4ccc(OC(F)(F)F)cc4)CCOc4cccnc43)ccc21 ; "HCO2H"] | (S)-1-methyl- N-(4-(4- (trifluoromethoxy) phenyl)- 3,4-dihydro- 2H-pyrano[3,2-b] pyridin-4- yl)-1H- indazole-5- carboxamide formate | 469.0 |
| 263 | 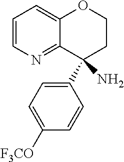 [SMILES: N[C@]1(c2ccc(OC(F)(F)F)cc2)CCOc2cccnc21] | 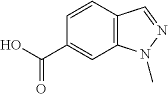 | 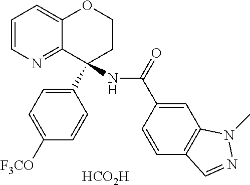 [SMILES: Cn1ncc2ccc(C(=O)N[C@]3(c4ccc(OC(F)(F)F)cc4)CCOc4cccnc43)cc21 ; "HCO2H"] | (S)-1-methyl- N-(4-(4- (trifluoromethoxy) phenyl)- 3,4-dihydro- 2H-pyrano[3,2-b] pyridin-4- yl)-1H- indazole-6- carboxamide formate | 469.0 |
| 264 | 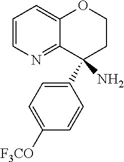 [SMILES: N[C@]1(c2ccc(OC(F)(F)F)cc2)CCOc2cccnc21] | 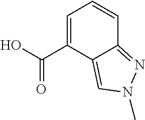 | 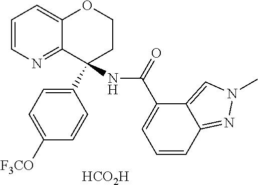 [SMILES: Cn1cc2c(C(=O)N[C@]3(c4ccc(OC(F)(F)F)cc4)CCOc4cccnc43)cccc2n1 ; "HCO2H"] | (S)-2-methyl- N-(4-(4- (trifluoromethoxy) phenyl)- 3,4-dihydro- 2H-pyrano[3,2-b] pyridin-4- yl)-2H- indazole-4- carboxamide formate | 469.4 |
| 265 | 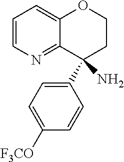 [SMILES: N[C@]1(c2ccc(OC(F)(F)F)cc2)CCOc2cccnc21] | 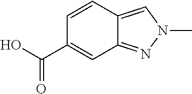 | 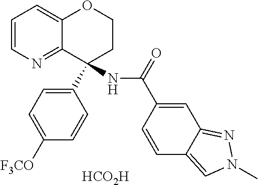 [SMILES: Cn1cc2ccc(C(=O)N[C@]3(c4ccc(OC(F)(F)F)cc4)CCOc4cccnc43)cc2n1 ; "HCO2H"] | (S)-2-methyl- N-(4-(4- (trifluoromethoxy) phenyl)- 3,4-dihydro- 2H-pyrano[3,2-b] pyridin-4- yl)-2H- indazole-6- carboxamide formate | 469.0 |
| 266 | 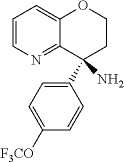 [SMILES: N[C@]1(c2ccc(OC(F)(F)F)cc2)CCOc2cccnc21] | 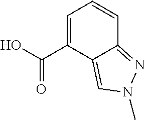 | 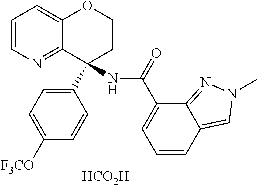 [SMILES: Cn1cc2cccc(C(=O)N[C@]3(c4ccc(OC(F)(F)F)cc4)CCOc4cccnc43)c2n1 ; "HCO2H"] | (S)-2-methyl- N-(4-(4- (trifluoromethoxy) phenyl)- 3,4-dihydro- 2H-pyrano[3,2-b] pyridin-4- yl)-2H- indazole-7- carboxamide formate | 469.0 |
| 267 | 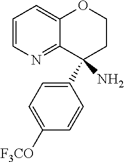 [SMILES: N[C@]1(c2ccc(OC(F)(F)F)cc2)CCOc2cccnc21] | 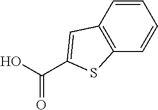 [SMILES: O=C(O)c1cc2ccccc2s1] | 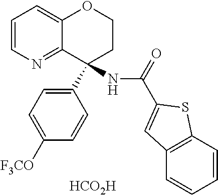 [SMILES: O=C(N[C@]1(c2ccc(OC(F)(F)F)cc2)CCOc2cccnc21)c1cc2ccccc2s1 ; "HCO2H"] | (S)-N-(4-(4- (trifluoromethoxy) phenyl)- 3,4-dihydro- 2H-pyrano[3,2-b] pyridin-4-yl) benzo[b]thiophene- 2-carboxamide formate | 471.0 |
| 268 | 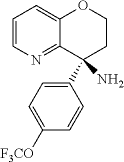 [SMILES: N[C@]1(c2ccc(OC(F)(F)F)cc2)CCOc2cccnc21] | 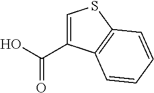 [SMILES: O=C(O)c1csc2ccccc12] | 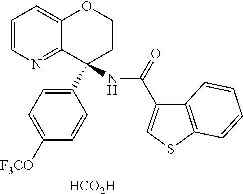 [SMILES: O=C(N[C@]1(c2ccc(OC(F)(F)F)cc2)CCOc2cccnc21)c1csc2ccccc12 ; "HCO2H"] | (S)-N-(4-(4- (trifluoromethoxy) phenyl)- 3,4-dihydro- 2H-pyrano[3,2-b] pyrid in-4-yl) benzo[b]thiophene- 3-carboxamide formate | 471.0 |
| 269 | 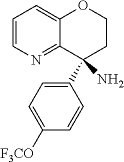 [SMILES: N[C@]1(c2ccc(OC(F)(F)F)cc2)CCOc2cccnc21] | 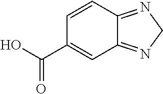 [SMILES: O=C(O)c1ccc2c(c1)=NCN=2] | 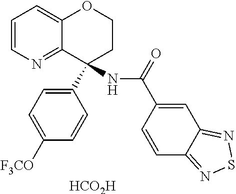 [SMILES: O=C(N[C@]1(c2ccc(OC(F)(F)F)cc2)CCOc2cccnc21)c1ccc2nsnc2c1 ; "HCO2H"] | (S)-N-(4-(4- (trifluoromethoxy) phenyl)- 3,4-dihydro- 2H-pyrano[3,2-b] pyridin-4-yl) benzo[c][1,2,5] thiadiazole-5- carboxamide formate | 473.0 |
| 270 | 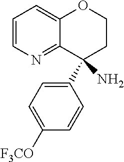 [SMILES: N[C@]1(c2ccc(OC(F)(F)F)cc2)CCOc2cccnc21] | 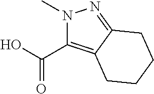 | 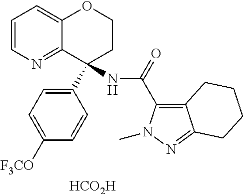 [SMILES: Cn1nc2c(c1C(=O)N[C@]1(c3ccc(OC(F)(F)F)cc3)CCOc3cccnc31)CCCC2 ; "HCO2H"] | (S)-2-methyl- N-(4-(4- (trifluoromethoxy) phenyl)- 3,4-dihydro- 2H-pyrano[3,2-b] pyridin-4- yl)-4,5,6,7- tetrahydro- 2H-indazole- 3-carboxamide formate | 473.4 |
| 271 | 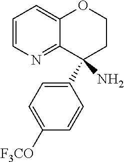 [SMILES: N[C@]1(c2ccc(OC(F)(F)F)cc2)CCOc2cccnc21] | 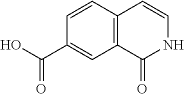 [SMILES: O=C(O)c1ccc2cc[nH]c(=O)c2c1] | 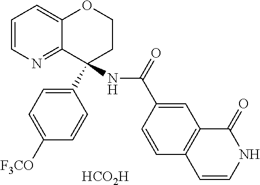 [SMILES: O=C(N[C@]1(c2ccc(OC(F)(F)F)cc2)CCOc2cccnc21)c1ccc2cc[nH]c(=O)c2c1 ; "HCO2H"] | (S)-1-oxo-N- (4-(4- (trifluoromethoxy) phenyl)- 3,4-dihydro- 2H-pyrano[3,2-b] pyridin-4- yl)-1,2- dihydroisoquinoline- 7-carboxamide formate | 482.2 |
| 272 | 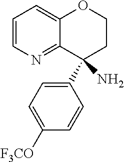 [SMILES: N[C@]1(c2ccc(OC(F)(F)F)cc2)CCOc2cccnc21] | 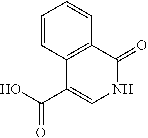 [SMILES: O=C(O)c1c[nH]c(=O)c2ccccc12] | 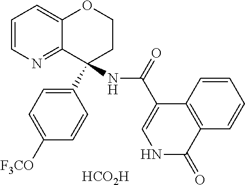 [SMILES: O=C(N[C@]1(c2ccc(OC(F)(F)F)cc2)CCOc2cccnc21)c1c[nH]c(=O)c2ccccc12 ; "HCO2H"] | (S)-1-oxo-N- (4-(4- (trifluoromethoxy) phenyl)- 3,4-dihydro- 2H-pyrano[3,2-b] pyridin-4- yl)-1,2- dihydroisoquinoline- 4-carboxamide formate | 480.2 |
| 273 | 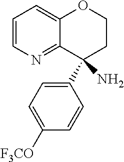 [SMILES: N[C@]1(c2ccc(OC(F)(F)F)cc2)CCOc2cccnc21] | 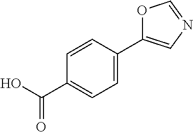 [SMILES: O=C(O)c1ccc(-c2cnco2)cc1] | 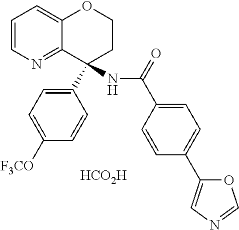 [SMILES: O=C(N[C@]1(c2ccc(OC(F)(F)F)cc2)CCOc2cccnc21)c1ccc(-c2cnco2)cc1 ; "HCO2H"] | (S)-4-(oxazol- 5-yl)-N-(4-(4- (trifluoromethoxy) phenyl)- 3,4-dihydro- 2H-pyrano[3,2-b] pyridin-4- yl)benzamide formate | 482.0 |
| 274 | 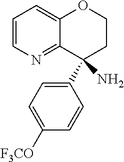 [SMILES: N[C@]1(c2ccc(OC(F)(F)F)cc2)CCOc2cccnc21] | 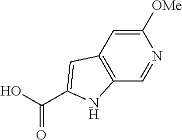 [SMILES: COc1cc2cc(C(=O)O)[nH]c2cn1] | 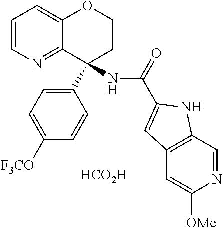 [SMILES: COc1cc2cc(C(=O)N[C@]3(c4ccc(OC(F)(F)F)cc4)CCOc4cccnc43)[nH]c2cn1 ; "HCO2H"] | (S)-5- methoxy-N- (4-(4- (trifluoromethoxy) phenyl)- 3,4-dihydro- 2H-pyrano[3,2-b] pyridin-4- yl)-1H- pyrrolo[2,3-c] pyridine-2- carboxamide formate | 485.2 |
| 275 | 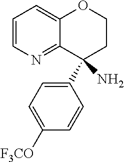 [SMILES: N[C@]1(c2ccc(OC(F)(F)F)cc2)CCOc2cccnc21] | 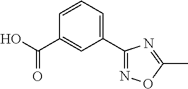 | 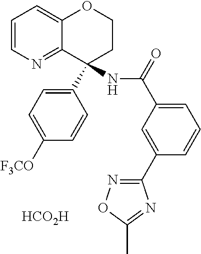 [SMILES: "HCO2H" ; Cc1nc(-c2cccc(C(=O)N[C@]3(c4ccc(OC(F)(F)F)cc4)CCOc4cccnc43)c2)no1] | (S)-3-(5- methyl-1,2,4- oxadiazol-3- yl)-N-(4-(4- (trifluoromethoxy) phenyl)- 3,4-dihydro- 2H-pyrano[3,2-b] pyridin-4- yl)benzamide formate | 497.0 |
| 276 | 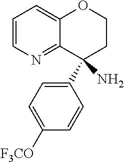 [SMILES: N[C@]1(c2ccc(OC(F)(F)F)cc2)CCOc2cccnc21] | 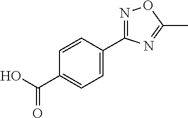 | 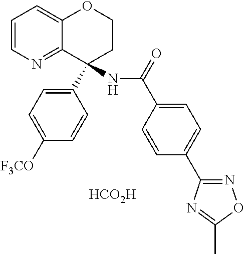 [SMILES: Cc1nc(-c2ccc(C(=O)N[C@]3(c4ccc(OC(F)(F)F)cc4)CCOc4cccnc43)cc2)no1 ; "HCO2H"] | (S)-4-(5- methyl-1,2,4- oxadiazol-3- yl)-N-(4-(4- (trifluoromethoxy) phenyl)- 3,4-dihydro- 2H-pyrano[3,2-b] pyridin-4- yl)benzamide formate | 497.0 |
| 277 | 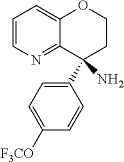 [SMILES: N[C@]1(c2ccc(OC(F)(F)F)cc2)CCOc2cccnc21] | 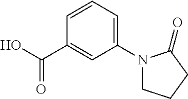 [SMILES: O=C(O)c1cccc(N2CCCC2=O)c1] | 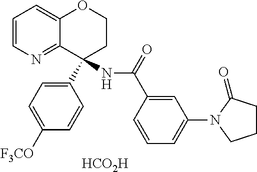 [SMILES: O=C(N[C@]1(c2ccc(OC(F)(F)F)cc2)CCOc2cccnc21)c1cccc(N2CCCC2=O)c1 ; "HCO2H"] | (S)-3-(2- oxopyrrolidin-1- yl)-N-(4-(4- (trifluoromethoxy) phenyl)- 3,4-dihydro- 2H-pyrano[3,2-b] pyridin-4- yl)benzamide formate | 498.0 |
| 278 | 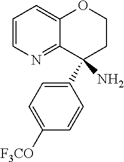 [SMILES: N[C@]1(c2ccc(OC(F)(F)F)cc2)CCOc2cccnc21] | 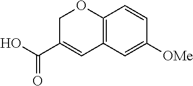 [SMILES: COc1ccc2c(c1)C=C(C(=O)O)CO2] | 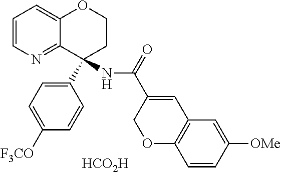 [SMILES: COc1ccc2c(c1)C=C(C(=O)N[C@]1(c3ccc(OC(F)(F)F)cc3)CCOc3cccnc31)CO2 ; "HCO2H"] | (S)-6- methoxy-N- (4-(4- (trifluoromethoxy) phenyl)- 3,4-dihydro- 2H-pyrano[3,2-b] pyridin-4- yl)-2H- chromene-3- carboxamide fonnate | 499.0 |
| 279 | 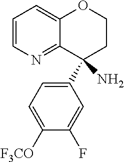 [SMILES: N[C@]1(c2ccc(OC(F)(F)F)c(F)c2)CCOc2cccnc21] | 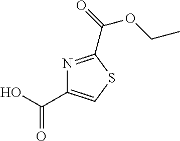 | 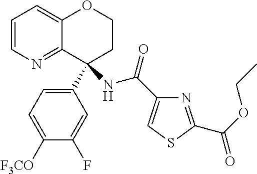 [SMILES: CCOC(=O)c1nc(C(=O)N[C@]2(c3ccc(OC(F)(F)F)c(F)c3)CCOc3cccnc32)cs1] | (S)-ethyl 4- ((4-(3-fluoro-4- (trifluoromethoxy) phenyl)- 3,4-dihydro- 2H-pyrano[3,2-b] pyridin-4- yl)carbamoyl) thiazole-2- carboxylate | 512.2 |
| 280 | 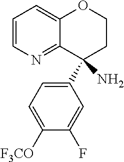 [SMILES: N[C@]1(c2ccc(OC(F)(F)F)c(F)c2)CCOc2cccnc21] | 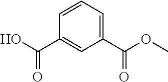 | 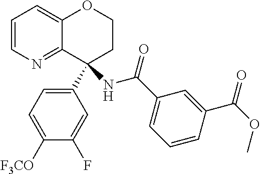 [SMILES: COC(=O)c1cccc(C(=O)N[C@]2(c3ccc(OC(F)(F)F)c(F)c3)CCOc3cccnc32)c1] | (S)-methyl 3- ((4-(3-fluoro-4- (trifluoromethoxy) phenyl)- 3,4-dihydro- 2H-pyrano[3,2-b] pyridin-4- yl)carbamoyl) benzoate | 491.0 |
| Inter- mediate 47 | 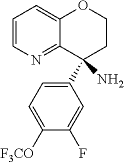 [SMILES: N[C@]1(c2ccc(OC(F)(F)F)c(F)c2)CCOc2cccnc21] | 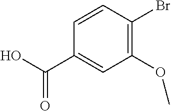 | 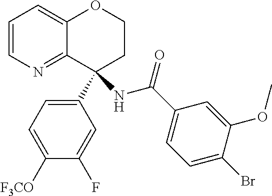 [SMILES: COc1cc(C(=O)N[C@]2(c3ccc(OC(F)(F)F)c(F)c3)CCOc3cccnc32)ccc1Br] | (S)-4-bromo- N-(4-(3- fluoro-4- (trifluoromethoxy) phenyl)- 3,4-dihydro- 2H- pyrano[3,2-b] pyridin-4-yl)-3- methoxybenzamide | 540.8 |
| 314 | 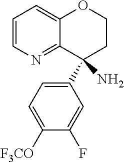 [SMILES: N[C@]1(c2ccc(OC(F)(F)F)c(F)c2)CCOc2cccnc21] | 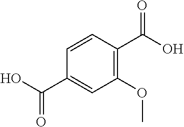 | 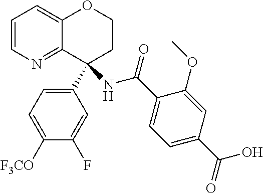 [SMILES: COc1cc(C(=O)O)ccc1C(=O)N[C@]1(c2ccc(OC(F)(F)F)c(F)c2)CCOc2cccnc21] | (S)-4-((4-(3- fluoro-4- (trifluoromethoxy) phenyl)- 3,4-dihydro- 2H-pyrano[3,2-b] pyridin-4- yl)carbamoyl)-3- methoxybenzoic acid | 507.0 |

### Example 281

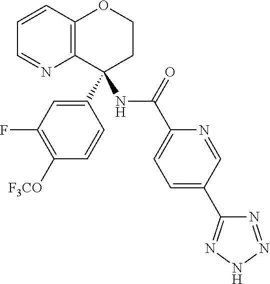 [SMILES: O=C(N[C@]1(c2ccc(OC(F)(F)F)c(F)c2)CCOc2cccnc21)c1ccc(-c2nn[nH]n2)cn1]

### (S)—N-(4-(3-fluoro-4-(trifluoromethoxy)phenyl)-3,4-dihydro-2H-pyrano[3,2-b]pyridin-4-yl)-5-(2H-tetrazol-5-yl)picolinamide

To a 20 mL vial with (S)-5-cyano-N-(4-(3-fluoro-4-(trifluoromethoxy)phenyl)-3,4-dihydro-2H-pyrano[3,2-b]pyridin-4-yl)picolinamide (100 mg, 0.218 mmol), sodium azide (17.0 mg, 0.262 mmol), and NH₄Cl (15.2 mg, 0.284 mmol) was added DMF (1 mL). The reaction was heated to 90° C. in an oil bath and stirred for 16 h. The reaction was cooled, diluted with DMF (1 mL), and the product was purified by reverse phase HPLC (Gemini Axia 50×250 mm C18, 10-100% CH₃CN, 0.1% TFA/H₂O, 0.1% TFA). The product-containing fractions were combined, and solvent was removed by lyophilization. The resulting solid was dissolved in MeOH and passed through a StratoSpheres® SPE PL-HCO₃ (6 mL, 0.36 mmol) cartridge with MeOH (5 mL) elution to freebase. Concentration of the filtrates gave the title compound as a white solid. MS (ESI, positive ion) m/z: 502.0 (MH+). ¹H NMR (300 MHz, MeOH-d₄) δ: 9.28 (d, J=1.3 Hz, 1H), 8.58 (dd, J=8.2, 2.2 Hz, 1H), 8.17-8.34 (m, 2H), 7.33-7.51 (m, 4H), 7.22-7.31 (m, 1H), 4.43 (dt, J=11.9, 4.1 Hz, 1H), 4.09 (td, J=11.5, 2.4 Hz, 1H), 3.33-3.41 (m, 1H), 3.13 (ddd, J=14.7, 11.2, 3.8 Hz, 1H).

### Example 282

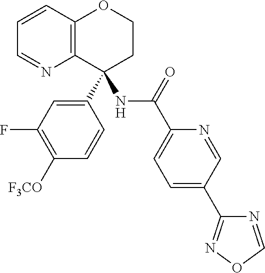 [SMILES: O=C(N[C@]1(c2ccc(OC(F)(F)F)c(F)c2)CCOc2cccnc21)c1ccc(-c2ncon2)cn1]

### (S)—N-(4-(3-fluoro-4-(trifluoromethoxy)phenyl)-3,4-dihydro-2H-pyrano[3,2-b]pyridin-4-yl)-5-(1,2,4-oxadiazol-3-yl)picolinamide

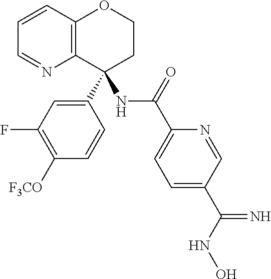 [SMILES: N=C(NO)c1ccc(C(=O)N[C@]2(c3ccc(OC(F)(F)F)c(F)c3)CCOc3cccnc32)nc1]

Step 1: (S)—N-(4-(3-fluoro-4-(trifluoromethoxy)phenyl)-3,4-dihydro-2H-pyrano[3,2-b]pyridin-4-yl)-5-(N-hydroxycarbamimidoyl)picolinamide

To a 20 mL vial containing (S)-5-cyano-N-(4-(3-fluoro-4-(trifluoromethoxy)phenyl)-3,4-dihydro-2H-pyrano[3,2-b]pyridin-4-yl)picolinamide (104 mg, 0.227 mmol), hydroxylamine hydrochloride (30 mg, 0.432 mmol), and NaHCO₃ (45.7 mg, 0.545 mmol), was added MeOH (2 mL). The vial was sealed, and the suspension was heated at 50° C. in a heating block and stirred for 2.5 h. The compound was diluted with H₂O (5 mL) and the solid was filtered off. The solid was washed with H₂O (3 mL) and dried under vacuum to give the title compound as a white solid which was used without further purification in the next step. MS (ESI, positive ion) m/z: 491.9 (MH+).

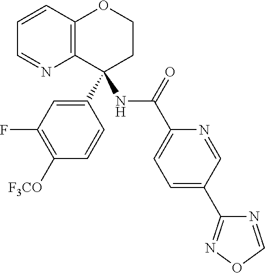 [SMILES: O=C(N[C@]1(c2ccc(OC(F)(F)F)c(F)c2)CCOc2cccnc21)c1ccc(-c2ncon2)cn1]

Step 2: (S)—N-(4-(3-fluoro-4-(trifluoromethoxy)phenyl)-3,4-dihydro-2H-pyrano[3,2-b]pyridin-4-yl)-5-(1,2,4-oxadiazol-3-yl)picolinamide

To (S)—N-(4-(3-fluoro-4-(trifluoromethoxy)phenyl)-3,4-dihydro-2H-pyrano[3,2-b]pyridin-4-yl)-5-(N-hydroxycarbamimidoyl)picolinamide (111 mg, 0.227 mmol) were added 1,4-dioxane (2 mL), triethoxymethane (0.113 mL, 0.681 mmol), and borontrifluoride diethyletherate (2.85 μl, 0.023 mmol). The resulting mixture was stirred at 75° C. in a heating block for 19 h. The reaction was cooled, the reaction diluted with DMF (1 mL), and the product was purified by reverse phase HPLC (Gemini Axia 50×250 mm C18, 10-100% CH₃CN, 0.1% TFA/H₂O, 0.1% TFA). The product-containing fractions were combined, and solvent was removed by lyophilization. The resulting solid was dissolved in MeOH and passed through a StratoSpheres® SPE PL-HCO₃ (6 mL, 0.36 mmol) cartridge with MeOH (5 mL) elution to freebase. Concentration of the filtrates gave the title compound as a white solid. MS (ESI, positive ion) m/z: 502.0 (MH+). ¹H NMR (300 MHz, MeOH-d₄) δ: 9.39 (s, 1H), 9.30 (dd, J=2.0, 0.7 Hz, 1H), 8.62 (dd, J=8.2, 2.2 Hz, 1H), 8.28 (t, J=2.9 Hz, 1H), 8.24 (dd, J=8.2, 0.7 Hz, 1H), 7.46 (dd, J=11.9, 2.3 Hz, 1H), 7.33-7.43 (m, 3H), 7.23-7.30 (m, 1H), 4.42 (dt, J=11.8, 4.1 Hz, 1H), 4.08 (td, J=11.6, 2.3 Hz, 1H), 3.32-3.43 (m, 1H), 3.13 (ddd, J=14.8, 11.2, 3.9 Hz, 1H).

### Example 283

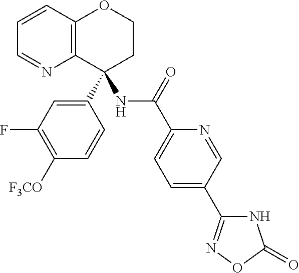 [SMILES: O=C(N[C@]1(c2ccc(OC(F)(F)F)c(F)c2)CCOc2cccnc21)c1ccc(-c2noc(=O)[nH]2)cn1]

### (S)—N-(4-(3-fluoro-4-(trifluoromethoxy)phenyl)-3,4-dihydro-2H-pyrano[3,2-b]pyridin-4-yl)-5-(5-oxo-4,5-dihydro-1,2,4-oxadiazol-3-yl)picolinamide

A suspension of (S)—N-(4-(3-fluoro-4-(trifluoromethoxy)phenyl)-3,4-dihydro-2H-pyrano[3,2-b]pyridin-4-yl)-5-(N-hydroxycarbamimidoyl)picolinamide (80 mg, 0.163 mmol), and di(1H-imidazol-1-yl)methanone (34.7 mg, 0.214 mmol) in 1,4-dioxane (1 mL) was heated in a sealed vial in a 100° C. heating block for 24 h. The reaction was cooled, diluted with DMF (1 mL), and the product purified by reverse phase HPLC (Gemini Axia 50×250 mm C18, 10-100% CH₃CN, 0.1% TFA/H₂O, 0.1% TFA). The product-containing fractions were combined, and solvent was removed by lyophilization. The resulting solid was dissolved in MeOH and passed through a StratoSpheres® SPE PL-HCO₃ (6 mL, 0.36 mmol) cartridge with MeOH (5 mL) elution to freebase. Concentration gave minimal product, the SPE column was reflushed with 4M HCl in Et₂O (5 mL), and the filtrates were concentrated to give the title compound as a yellow solid. MS (ESI, positive ion) m/z: 517.8 (MH+). ¹H NMR (300 MHz, MeOH-d₄) δ: 8.67 (br. s., 1H), 8.25 (d, J=3.4 Hz, 1H), 8.00 (d, J=8.6 Hz, 1H), 7.87 (d, J=8.0 Hz, 1H), 7.32-7.58 (m, 4H), 7.19 (d, J=9.2 Hz, 1H), 4.32-4.52 (m, 1H), 4.03-4.20 (m, 1H), 3.04-3.22 (m, 1H), 2.89 (m, 1H).

### Example 284

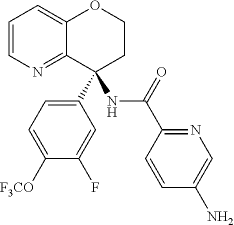 [SMILES: Nc1ccc(C(=O)N[C@]2(c3ccc(OC(F)(F)F)c(F)c3)CCOc3cccnc32)nc1]

### (S)-5-amino-N-(4-(3-fluoro-4-(trifluoromethoxy)phenyl)-3,4-dihydro-2H-pyrano[3,2-b]pyridin-4-yl)picolinamide

To a solution of (S)—N-(4-(3-fluoro-4-(trifluoromethoxy)phenyl)-3,4-dihydro-2H-pyrano[3,2-b]pyridin-4-yl)-5-nitropicolinamide (237 mg, 0.495 mmol) in MeOH (2 mL) was added a mixture of 10% palladium on activated carbon (5.27 mg, 0.050 mmol) in EtOAc (0.3 mL). After the addition, the mixture was stirred at room temperature overnight under H₂. The mixture was filtered through Celite® brand filter agent, and the Celite® brand filter agent was washed with MeOH (2×5 mL). The combined filtrates were concentrated, and the residue was dissolved in DCM (2 mL). The mixture was then purified by silica gel column chromatography (0-100% EtOAc/hexane) to give the title compound as a yellow solid. MS (ESI, positive ion) m/z: 449.1 (MH+). ¹H NMR (300 MHz, MeOH— d₄) δ: 8.24 (dd, J=3.6, 2.2 Hz, 1H), 7.96 (d, J=2.2 Hz, 1H), 7.76 (d, J=8.6 Hz, 1H), 7.33-7.48 (m, 4H), 7.17-7.28 (m, 1H), 7.04 (dd, J=8.5, 2.6 Hz, 1H), 4.31-4.46 (m, 1H), 4.02-4.18 (m, 1H), 3.16-3.24 (m, 2H).

### Example 285

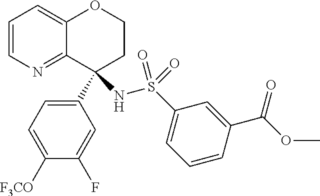 [SMILES: COC(=O)c1cccc(S(=O)(=O)N[C@]2(c3ccc(OC(F)(F)F)c(F)c3)CCOc3cccnc32)c1]

### (S)-methyl 3-(N-(4-(3-fluoro-4-(trifluoromethoxy)phenyl)-3,4-dihydro-2H-pyrano[3,2-b]pyridin-4-yl)sulfamoyl)benzoate

To a solution of (S)-4-(3-fluoro-4-(trifluoromethoxy)phenyl)-3,4-dihydro-2H-pyrano[3,2-b]pyridin-4-amine hydrochloride (150 mg, 0.411 mmol) in DCM (2.6 mL) were added methyl 3-(chlorosulfonyl)benzoate (174 mg, 0.740 mmol) and TEA (0.114 mL, 0.823 mmol). The resulting mixture was stirred at room temperature overnight. The mixture was then purified by silica gel column chromatography using flash chromatography instrument (0-100% EtOAc in hexanes) to give the title compound as a light yellow solid. MS (ESI, positive ion) m/z: 527.0 (MH+). ¹H NMR (300 MHz, MeOH-d₄) δ: 8.05-8.12 (m, 2H), 8.01-8.04 (m, 1H), 7.82-7.92 (m, 1H), 7.45-7.53 (m, 1H), 7.20-7.34 (m, 2H), 7.07-7.18 (m, 3H), 4.29-4.43 (m, 1H), 3.98-4.10 (m, 1H), 3.96 (s, 3H), 2.82-2.94 (m, 2H).

### Example 286

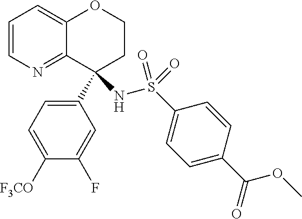 [SMILES: COC(=O)c1ccc(S(=O)(=O)N[C@]2(c3ccc(OC(F)(F)F)c(F)c3)CCOc3cccnc32)cc1]

### (S)-methyl 4-(N-(4-(3-fluoro-4-(trifluoromethoxy)phenyl)-3,4-dihydro-2H-pyrano[3,2-b]pyridin-4-yl)sulfamoyl)benzoate

The title compound was prepared in an analogous manner to Example 285 using methyl 4-(chlorosulfonyl)-benzoate to give the title compound. MS (ESI, positive ion) m/z: 527.0 (MH+). ¹H NMR (300 MHz, MeOH-d₄) δ: 8.04 (dd, J=4.4, 1.5 Hz, 1H), 7.92-7.98 (m, 2H), 7.63-7.74 (m, 2H), 7.22-7.33 (m, 2H), 7.06-7.20 (m, 3H), 4.23-4.45 (m, 1H), 3.97-4.10 (m, 1H), 3.95 (s, 3H), 2.78-2.93 (m, 2H)

### Example 287

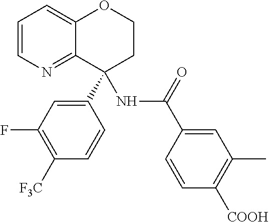 [SMILES: Cc1cc(C(=O)N[C@]2(c3ccc(C(F)(F)F)c(F)c3)CCOc3cccnc32)ccc1C(=O)O]

### (S)-4-((4-(3-fluoro-4-(trifluoromethyl)phenyl)-3,4-dihydro-2H-pyrano[3,2-b ]pyridin-4-yl)carbamoyl)-2-methylbenzoic acid

To a stirred mixture of (S)-4-(3-fluoro-4-(trifluoromethyl)phenyl)-3,4-dihydro-2H-pyrano[3,2-b]pyridin-4-amine hydrochloride (0.100 g, 0.287 mmol), 4-(methoxycarbonyl)-3-methylbenzoic acid (0.067 g, 0.344 mmol), and DIPEA (0.160 ml, 0.918 mmol) in DMF (3 mL), was added HATU (0.120 g, 0.315 mmol). After stirring for 2 h, H₂O was added and the mixture was extracted with DCM (3×). The combined organic layers were dried over Na₂SO₄, concentrated and purified by flash column chromatography (0-50% EtOAc in hexanes). The resulting material was dissolved in MeOH (4 mL), NaOH (5N in H₂O (0.287 ml, 1.434 mmol)) was added, and the reaction was heated at 55° C. for 2 h. The reaction mixture was cooled, concentrated, diluted with H₂O and acidified with 5N HCl. The tan solid was collected, and purified by reverse phase HPLC. The pure fractions were concentrated to minimal H₂O, neutralized with saturated aqueous NaHCO₃. The solid was collected and dried to give the title compound. MS (ESI pos. ion) m/z: 475 (MH+). ¹H NMR (400 MHz, DMSO-d₆) δ: 13.03 (br. s., 1H), 9.01 (br. s., 1H), 8.10 (d, J=3.1 Hz, 1H), 7.63-7.92 (m, 3H), 7.52 (d, J=12.9 Hz, 1H), 7.16-7.44 (m, 4H), 4.26-4.42 (m, 1H), 4.20 (br. s., 1H), 3.22 (br. s., 1H), 2.82-2.98 (m, 1H), 2.51 (s, 3H).

### Example 288

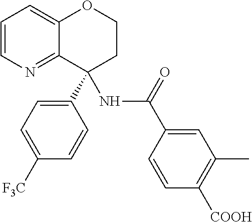 [SMILES: Cc1cc(C(=O)N[C@]2(c3ccc(C(F)(F)F)cc3)CCOc3cccnc32)ccc1C(=O)O]

### (S)-2-methyl-4-((4-(4-(trifluoromethyl)phenyl)-3,4-dihydro-2H-pyrano[3,2-b]pyridin-4-yl)carbamoyl)benzoic acid

The title compound was prepared using the same method as described in Example 287 using (S)-4-(4-(trifluoromethyl)phenyl)-3,4-dihydro-2H-pyrano[3,2-b]pyridin-4-amine hydrochloride. MS (ESI pos. ion) m/z: 457 (MH+). ¹H NMR (400 MHz, MeOH-d₄) δ: 8.19 (dd, J=4.3, 1.8 Hz, 1H), 7.81 d, J=8.0 Hz, 1H), 7.61-7.72 (m, 4H), 7.51-7.61 (m, 2H), 7.24-7.40 (m, 2H), 4.32-4.46 (m, 1H), 4.01-4.13 (m, 1H), 3.34-3.43 (m, 2H), 2.99-3.10 (m, 1H), 2.58 (s, 3H).

### Example 289

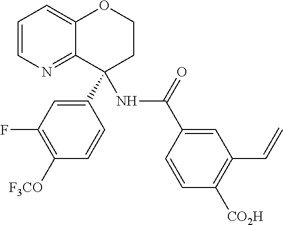 [SMILES: C=Cc1cc(C(=O)N[C@]2(c3ccc(OC(F)(F)F)c(F)c3)CCOc3cccnc32)ccc1C(=O)O]

### (S)-4-((4-(3-fluoro-4-(trifluoromethoxy)phenyl)-3,4-dihydro-2H-pyrano[3,2-b]pyridin-4-yl)carbamoyl)-2-vinylbenzoic acid

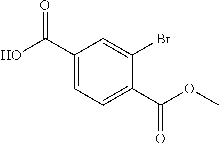

Step 1: 3-bromo-4-(methoxycarbonyl)benzoic acid

To a solution of methyl 2-bromo-4-methylbenzoate (2.60 g, 11.35 mmol) in DCM (50 mL) and H₂O (6 mL), were added oxone, monopersulfate compound (8.37 g, 13.62 mmol) and KBr (1.621 g, 13.62 mmol). The resulting mixture was irradiated with a fluorescent lamp and stirred under air for 24 h. The reaction was quenched with saturated Na₂SO₃ (50 mL), 10% aqueous citric acid (80 mL) was added, and the solution was extracted with EtOAc (3×50 mL). The combined organic layers were washed with brine, dried (MgSO₄), and concentrated in vacuo. Purification by automated flash chromatography (10-100% (1% AcOH in EtOAc in hexanes) gave the title compound as a white solid. ¹H NMR (300 MHz, DMSO-d₆) δ: 13.60 (br. s., 1H), 8.17 (d, J=1.5 Hz, 1H), 8.01 (dd, J=8.0, 1.6 Hz, 1H), 7.86 (d, J=8.0 Hz, 1H), 3.89 (s, 3H)

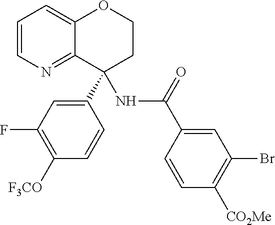 [SMILES: COC(=O)c1ccc(C(=O)N[C@]2(c3ccc(OC(F)(F)F)c(F)c3)CCOc3cccnc32)cc1Br]

Step 2: (S)-methyl 2-bromo-4-((4-(3-fluoro-4-(trifluoromethoxy)phenyl)-3,4-dihydro-2H-pyrano[3,2-b]pyridin-4-yl)carbamoyl)benzoate

To a suspension of (S)-4-(3-fluoro-4-(trifluoromethoxy)phenyl)-3,4-dihydro-2H-pyrano[3,2-b]pyridin-4-amine hydrochloride (1056 mg, 2.90 mmol), and 3-bromo-4-(methoxycarbonyl)benzoic acid (900 mg, 3.47 mmol) in DCM (20 mL) at room temperature, was added DIPEA (1.513 mL, 8.69 mmol) and HATU (1101 mg, 2.90 mmol). The yellow solution containing a small amount of floating precipitate, was stirred for 72 h at room temperature under air. The reaction was diluted with saturated NaHCO₃ (50 mL) and EtOAc (50 mL), and the organic layer was separated. The aqueous layer was extracted with EtOAc (50 mL), and the combined organic layers were dried (MgSO₄) and concentrated to give a residual brown oil. Purification by flash chromatography (5-50% EtOAc in hexanes) gave the title compound as a white foam. MS (ESI pos. ion) m/z: 568.8, 570.8 (MH+). ¹H NMR (300 MHz, DMSO-d₆) δ: 9.16 (s, 1H), 8.15 (d, J=1.5 Hz, 1H), 8.12 (dd, J=4.2, 1.8 Hz, 1H), 7.88 (dd, J=8.0, 1.6 Hz, 1H), 7.80 (d, J=8.2 Hz, 1H), 7.42-7.60 (m, 2H), 7.15-7.36 (m, 3H), 4.26-4.41 (m, 1H), 4.05-4.18 (m, 1H), 3.88 (s, 3H), 3.25-3.35 (m, 1H), 2.67-2.86 (m, 1H)

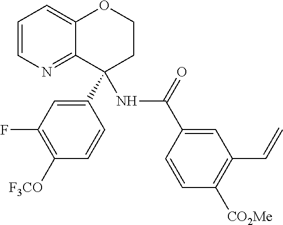 [SMILES: C=Cc1cc(C(=O)N[C@]2(c3ccc(OC(F)(F)F)c(F)c3)CCOc3cccnc32)ccc1C(=O)OC]

Step 3: (S)-methyl 4-((4-(3-fluoro-4-(trifluoromethoxy)phenyl)-3,4-dihydro-2H-pyrano[3,2-b]pyridin-4-yl)carbamoyl)-2-vinylbenzoate

A mixture of (S)-methyl 2-bromo-4-((4-(3-fluoro-4-(trifluoromethoxy)phenyl)-3,4-dihydro-2H-pyrano[3,2-b]pyridin-4-yl)carbamoyl)benzoate (0.208 g, 0.365 mmol), tri-n-butyl(vinyl)tin (0.139 ml, 0.438 mmol), and bis(tri-tert-butylphosphine)-palladium (0) (19 mg, 0.037 mmol) in THF (2 mL) was heated at 90° C. for 2 h. The reaction mixture was cooled, saturated aqueous KF was added, and the reaction was stirred for 10 min. The resulting mixture was extracted with EtOAc (3×). The combined organic layers were dried over MgSO₄, concentrated, and purified by flash column chromatography (0-50% EtOAc in hexanes) to give the title compound. MS (ESI pos. ion) m/z: 517.0 (MH+).

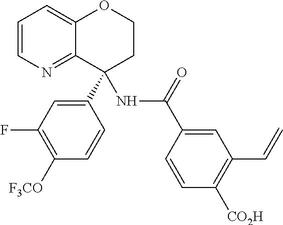 [SMILES: C=Cc1cc(C(=O)N[C@]2(c3ccc(OC(F)(F)F)c(F)c3)CCOc3cccnc32)ccc1C(=O)O]

Step 4: (S)-4-((4-(3-fluoro-4-(trifluoromethoxy)phenyl)-3,4-dihydro-2H-pyrano[3,2-b]pyridin-4-yl)carbamoyl)-2-vinylbenzoic acid

(S)-methyl 4-((4-(3-fluoro-4-(trifluoromethoxy)phenyl)-3,4-dihydro-2H-pyrano[3,2-b]pyridin-4-yl)carbamoyl)-2-vinylbenzoate (113 mg, 0.219 mmol) was dissolved in MeOH/THF (1:4, 5 mL), and NaOH (5N in H₂O, 0.219 ml, 1.096 mmol) was added. After stirring for 24 h at room temperature, the reaction mixture was concentrated and acidified. The off-white solid was collected, washed with H₂O, and dried to give the title compound. MS (ESI pos. ion) m/z: 503 (MH+). ¹H NMR (400 MHz, DMSO-d₆) δ: 13.27 (br. s., 1H), 9.10 (br. s., 1H), 8.13 (br. s., 1H), 8.02 (br. s., 1H), 7.79 (d, J=5.7 Hz, 2H), 7.45-7.60 (m, 2H), 7.34-7.45 (m, 1H), 7.11-7.34 (m, 3H), 5.81 (d, J=17.4 Hz, 1H), 5.38 (d, J=10.8 Hz, 1H), 4.34 (m, 1H), 4.13 (m, 1H), 3.35 (m, 1H), 2.84 (m, 1H).

### Example 290

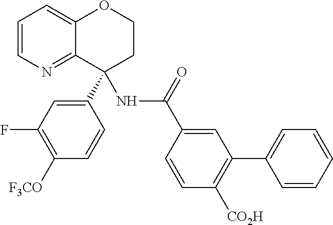 [SMILES: O=C(N[C@]1(c2ccc(OC(F)(F)F)c(F)c2)CCOc2cccnc21)c1ccc(C(=O)O)c(-c2ccccc2)c1]

### (S)-5-((4-(3-fluoro-4-(trifluoromethoxy)phenyl)-3,4-dihydro-2H-pyrano[3,2-b]pyridin-4-yl)carbamoyl)-[1,1′-biphenyl]-2-carboxylic acid

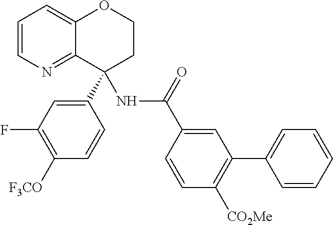 [SMILES: COC(=O)c1ccc(C(=O)N[C@]2(c3ccc(OC(F)(F)F)c(F)c3)CCOc3cccnc32)cc1-c1ccccc1]

Step 1: (S)-methyl 5-((4-(3-fluoro-4-(trifluoromethoxy)phenyl)-3,4-dihydro-2H-pyrano[3,2-b]pyridin-4-yl)carbamoyl)-[1,1′-biphenyl]-2-carboxylate

A mixture of (S)-methyl 2-bromo-4-((4-(3-fluoro-4-(trifluoromethoxy)phenyl)-3,4-dihydro-2H-pyrano[3,2-b]pyridin-4-yl)carbamoyl)benzoate (0.115 g, 0.202 mmol), phenylboronic acid (0.032 g, 0.263 mmol), Na₂CO₃ (0.075 g, 0.707 mmol), and bis(tri-tert-butylphosphine)palladium (0) (10.32 mg, 0.020 mmol) in THF/H₂O (10:1, 3.3 mL) was heated at 100° C. for 2 h. The reaction mixture was cooled, diluted with EtOAc, and washed with H₂O, saturated aqueous NaHCO₃, and brine. The organic layer was dried over MgSO₄, concentrated, and purified by flash column chromatography (0-40% EtOAc in hexanes) to give the title compound. MS (ESI pos. ion) m/z: 567 (MH+).

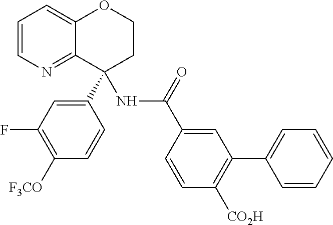 [SMILES: O=C(N[C@]1(c2ccc(OC(F)(F)F)c(F)c2)CCOc2cccnc21)c1ccc(C(=O)O)c(-c2ccccc2)c1]

Step 2: S)-5-(4-(3-fluoro-4-(trifluoromethoxy)phenyl)-3,4-dihydro-2H-pyrano[3,2-b]pyridin-4-yl)carbamoyl)-[1,1′-biphenyl]-2-carboxylic acid

The title compound was prepared using the method described Example 289 Step 4.

MS (ESI pos. ion) m/z: 553 (MH+). ¹H NMR (400 MHz, DMSO-d₆) δ: 13.02 (br. s., 1H), 9.15 (s, 1H), 8.12 (dd, J=4.3, 1.4 Hz, 1H), 7.85-7.90 (m, 1H), 7.84 (s, 1H), 7.77 (d, J=8.0 Hz, 1H), 7.35-7.60 (m, 6H), 7.21-7.34 (m, 3H), 4.35 (m, 1H), 4.16 (m, 1H), 3.32 (m, 1H), 2.84 (m, 1H).

### Example 291

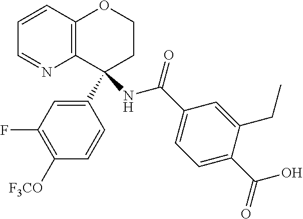 [SMILES: CCc1cc(C(=O)N[C@]2(c3ccc(OC(F)(F)F)c(F)c3)CCOc3cccnc32)ccc1C(=O)O]

### (S)-2-ethyl-4-((4-(3-fluoro-4-(trifluoromethoxy)phenyl)-3,4-dihydro-2H-pyrano[3,2-b]pyridin-4-yl)carbamoyl)benzoic acid

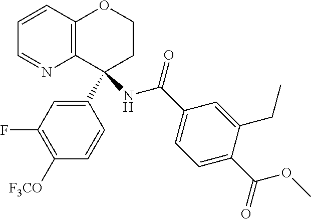 [SMILES: CCc1cc(C(=O)N[C@]2(c3ccc(OC(F)(F)F)c(F)c3)CCOc3cccnc32)ccc1C(=O)OC]

Step 1: (S)-methyl 2-ethyl-4-((4-(3-fluoro-4-(trifluoromethoxy)phenyl)-3,4-dihydro-2H-pyrano[3,2-b]pyridin-4-yl)carbamoyl)benzoate

To a mixture of 3-ethyl-4-(methoxycarbonyl)benzoic acid (0.349 g, 1.675 mmol), (S)-4-(3-fluoro-4-(trifluoromethoxy)phenyl)-3,4-dihydro-2H-pyrano[3,2-b]pyridin-4-amine hydrochloride (0.500 g, 1.523 mmol), DIPEA (0.346 ml, 1.980 mmol) in DMF (5 mL) was added HATU (0.695 g, 1.828 mmol). The mixture was stirred at room temperature overnight. The mixture was then diluted with H₂O and extracted with DCM (3×). The combined organic layers were dried over Na₂SO₄, concentrated in vacuo, and the residue was purified by silica gel flash chromatography (0-50% EtOAc in hexanes) to give the title compound. ¹H NMR (400 MHz, DMSO-d₆) δ: 9.01 (s, 1H), 8.13 (dd, J=4.2, 1.7 Hz, 1H), 7.75-7.83 (m, 2H), 7.66-7.75 (m, 1H), 7.45-7.57 (m, 2H), 7.17-7.33 (m, 3H), 4.34 (ddd, J=11.2, 7.9, 2.8 Hz. 1H), 4.12 (ddd, J=11.2, 7.9, 2.7 Hz, 1H), 3.85 (s, 3H), 3.22-3.30 (m, 1H), 2.90 (q, J=7.6 Hz, 2H), 2.77-2.87 (m, 1H), 1.16 (t, J=7.4 Hz, 3H).

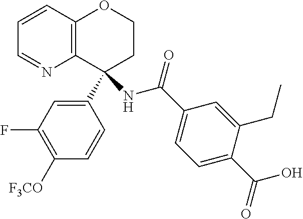 [SMILES: CCc1cc(C(=O)N[C@]2(c3ccc(OC(F)(F)F)c(F)c3)CCOc3cccnc32)ccc1C(=O)O]

Step 2: (S)-2-ethyl-4-((4-(3-fluoro-4-(trifluoromethoxy)phenyl)-3,4-dihydro-2H-pyrano[3,2-b]pyridin-4-yl)carbamoyl)benzoic acid

A mixture of (S)-methyl 2-ethyl-4-((4-(3-fluoro-4-(trifluoromethoxy)phenyl)-3,4-dihydro-2H-pyrano[3,2-b]pyridin-4-yl)carbamoyl)benzoate (12.79 g, 24.67 mmol) and aqueous NaOH (5M in H₂O, 24.67 ml, 123 mmol) in THF/MeOH (1:1, 150 mL) was stirred at room temperature for 36 h. The reaction mixture was concentrated in vacuo, diluted with H₂O, cooled in an ice bath, and acidified with 5N HCl to pH=3. The resulting solid was filtered, washed with H₂O, and dried under high vacuum over the weekend to give the title compound. MS (ESI, positive ion) m/z: 505.1 (MH+). ¹H NMR (400 MHz, DMSO-d₆) δ: 13.07 (br. s., 1H), 8.98 (s, 1H), 8.13 (dd, J=4.2, 1.7 Hz, 1H), 7.76-7.82 (m, 1H), 7.67-7.75 (m, 2H), 7.42-7.58 (m, 2H), 7.19-7.35 (m, 3H), 4.34 (ddd, J=11.1, 7.9, 2.7 Hz, 1H), 4.13 (ddd, J=11.2, 7.9, 2.6 Hz, 1H), 3.27 (td, J=7.1, 2.9 Hz, 1H), 2.93 (q, J=7.4 Hz, 2H), 2.86 (ddd, J=14.2, 7.7, 2.7 Hz, 1H), 1.16 (t, J=7.4 Hz, 3H).

### Example 292

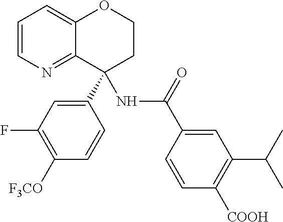 [SMILES: CC(C)c1cc(C(=O)N[C@]2(c3ccc(OC(F)(F)F)c(F)c3)CCOc3cccnc32)ccc1C(=O)O]

### (S)-4-((4-(3-fluoro-4-(trifluoromethoxy)phenyl)-3,4-dihydro-2H-pyrano[3,2-b]pyridin-4-yl)carbamoyl)-2-isopropylbenzoic acid

A mixture of (S)-methyl 2-bromo-4-((4-(3-fluoro-4-(trifluoromethoxy)phenyl)-3,4-dihydro-2H-pyrano[3,2-b]pyridin-4-yl)carbamoyl)benzoate (0.201 g, 0.353 mmol), 2-propylzinc bromide (0.5M in THF, 0.777 ml, 0.388 mmol), and bis(tri-tert-butylphosphine)palladium (0) (0.018 g, 0.035 mmol) in THF (5 mL) was heated at 90° C. in a 20 mL sealed tube for 16 h. The reaction mixture was cooled, quenched with saturated aqueous NH₄Cl, and extracted with EtOAc (3×). The combined organic layers were dried over Na₂SO₄, concentrated and purified by flash column chromatography (0-50% EtOAc in hexanes). The resulting material was dissolved in MeOH (4 mL), and NaOH (5N, 0.353 ml, 1.765 mmol) was added. The mixture was stirred at room temperature for 24 h, concentrated, and acidified. The solid was collected, washed with H₂O, dried to give the title compound. MS (ESI pos. ion) m/z: 519 (MH+). ¹H NMR (400 MHz, DMSO-d₆) δ: 13.13 (br. s., 1H), 9.05 (s, 1H), 8.15 (dd, J=4.2, 1.7 Hz, 1H), 7.80-7.85 (m, 1H), 7.74 (dd, J=8.0, 1.6 Hz, 1H), 7.66 (d, J=8.2 Hz, 1H), 7.46-7.59 (m, 2H), 7.21-7.36 (m, 3H), 4.35 (ddd, J=11.2, 7.9, 2.8 Hz, 1H), 4.08-4.18 (m, 1H), 3.67 (dt, J=13.7, 6.9 Hz, 1H), 3.20-3.32 (m, 1H), 2.81-2.93 (m, 1H), 1.22 (d, J=6.8 Hz, 6H).

### Example 293

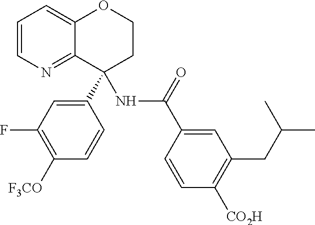 [SMILES: CC(C)Cc1cc(C(=O)N[C@]2(c3ccc(OC(F)(F)F)c(F)c3)CCOc3cccnc32)ccc1C(=O)O]

### (S)-4-((4-(3-fluoro-4-(trifluoromethoxy)phenyl)-3,4-dihydro-2H-pyrano[3,2-b]pyridin-4-yl)carbamoyl)-2-isobutylbenzoic acid

The title compound was prepared using the same method as described for Example 292 using isobutylzinc bromide. MS (ESI pos. ion) m/z: 533 (MH+). ¹H NMR (400 MHz, DMSO-d₆) δ: 13.02 (br. s., 1H), 8.96 (s, 1H), 8.12 (dd, J=4.2, 1.7 Hz, 1H), 7.77 (d, J=8.0 Hz, 1H), 7.71 (dd, J=8.1, 1.7 Hz, 1H), 7.64 (d, J=1.4 Hz, 1H), 7.43-7.56 (m, 2H), 7.24-7.34 (m, 2H), 7.17-7.24 (m, 1H), 4.22-4.46 (m, 1H), 4.12 (ddd, J=11.2, 8.0, 2.7 Hz, 1H), 3.17-3.28 (m, 1H), 2.76-2.97 (m, 3H), 1.67-1.91 (m, 1H), 0.83 (dd, J=6.7, 2.0 Hz, 6H).

### Example 294

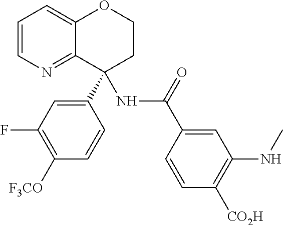 [SMILES: CNc1cc(C(=O)N[C@]2(c3ccc(OC(F)(F)F)c(F)c3)CCOc3cccnc32)ccc1C(=O)O]

### (S)-4-((4-(3-fluoro-4-(trifluoromethoxy)phenyl)-3,4-dihydro-2H-pyrano[3,2-b]pyridin-4-yl)carbamoyl)-2-(methylamino)benzoic acid

A mixture of (S)-methyl 2-amino-4-((4-(3-fluoro-4-(trifluoromethoxy)phenyl)-3,4-dihydro-2H-pyrano[3,2-b]pyridin-4-yl)carbamoyl)benzoate (0.100 g, 0.198 mmol), trimethyl orthoformate (0.173 ml, 1.583 mmol), formaldehyde (37% wt in water, 0.044 ml, 0.594 mmol) in DCE (4 mL) was stirred at room temperature for 1 h. NaBH(OAc)₃ (0.147 ml, 0.989 mmol) was added, and the resulting mixture was stirred at room temperature for 24 h. Formaldehyde (37% wt in water (0.044 ml, 0.594 mmol)) and NaBH(OAc)₃ (0.147 ml, 0.989 mmol) were added again, and the mixture was stirred for another 6 h. The reaction was then quenched with H₂O, stirred, and extracted with DCM (3×). The combined organic layers were dried over Na₂SO₄, concentrated and purified by flash column chromatography (0-50% EtOAc in hexanes) to give the ester which was dissolved in MeOH (2 mL) and treated with 5N NaOH (0.200 mL) and stirred for 24 h. The reaction mixture was concentrated, taken up in H₂O, and acidified with 5N HCl. The solid was collected, washed with H₂O, and dried to give the title compound. MS (ESI pos. ion) m/z: 506 (MH+). ¹H NMR (400 MHz, DMSO-d₆) δ: 8.94 (s, 1H), 8.15 (d, J=2.5 Hz, 1H), 7.82 (d, J=8.2 Hz, 1H), 7.46-7.62 (m, 2H), 7.21-7.37 (m, 3H), 7.04 (s, 1H), 6.97 (d, J=8.6 Hz, 1H), 4.35 (t, J=8.2 Hz, 1H), 4.15 (t, J=8.1 Hz, 1H), 3.13-3.27 (m, 1H), 2.75-3.05 (m, 1H), 2.87 (s, 3H).

### Example 295

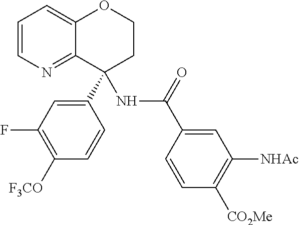 [SMILES: COC(=O)c1ccc(C(=O)N[C@]2(c3ccc(OC(F)(F)F)c(F)c3)CCOc3cccnc32)cc1NC(C)=O]

### (S)-methyl 2-acetamido-4-((4-(3-fluoro-4-(trifluoromethoxy)phenyl)-3,4-dihydro-2H-pyrano[3,2-b]pyridin-4-yl)carbamoyl)benzoate

A mixture of (S)-methyl 2-amino-4-((4-(3-fluoro-4-(trifluoromethoxy)phenyl)-3,4-dihydro-2H-pyrano[3,2-b]pyridin-4-yl)carbamoyl)benzoate (0.100 g, 0.198 mmol), pyridine (0.080 ml, 0.989 mmol), and acetic anhydride (0.093 ml, 0.989 mmol) in DCM (2 mL) was stirred at room temperature for 48 h. The reaction mixture was concentrated to dryness and purified by flash column chromatography (0-60% EtOAc in hexanes) to give the title compound. MS (ESI pos. ion) m/z: 548 (MH+). ¹H NMR (400 MHz, DMSO-d₆) δ: 10.44 (s, 1H), 9.04 (s, 1H), 8.40 (d, J=1.6 Hz, 1H), 8.13 (dd, J=4.2, 1.7 Hz, 1H), 7.83-7.92 (m, 1H), 7.60 (dd, J=8.2, 1.6 Hz, 1H), 7.42-7.56 (m, 2H), 7.25-7.33 (m, 2H), 7.19-7.25 (m, 1H), 4.34 (ddd, J=11.2, 7.8, 2.8 Hz, 1H), 4.12 (ddd, J=11.3, 8.1, 2.7 Hz, 1H), 3.84 (s, 3H), 3.24 (ddd, J=14.3, 7.9, 2.8 Hz, 1H), 2.89 (ddd, J=14.3, 7.5, 2.8 Hz, 1H), 2.10 (s, 3H).

### Example 296

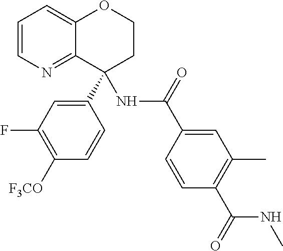 [SMILES: CNC(=O)c1ccc(C(=O)N[C@]2(c3ccc(OC(F)(F)F)c(F)c3)CCOc3cccnc32)cc1C]

### (S)—N4-(4-(3-fluoro-4-(trifluoromethoxy)phenyl)-3,4-dihydro-2H-pyrano[3,2-b]pyridin-4-yl)-N1,2-dimethylterephthalamide

A mixture of (S)-4-((4-(3-fluoro-4-(trifluoromethoxy)phenyl)-3,4-dihydro-2H-pyrano[3,2-b]pyridin-4-yl)carbamoyl)-2-methylbenzoic acid (3.99 ml, 0.271 mmol), methylamine (0.163 ml, 0.325 mmol), DIPEA (0.062 ml, 0.353 mmol), and HATU (0.113 g, 0.298 mmol) in DMF (4 mL) was stirred at room temperature for 16 h. H₂O was then added, and the mixture was extracted with DCM (3×). The combined organic layers were dried over Na₂SO₄, concentrated, and purified by flash chromatography (0-60% EtOAc in hexanes) to give the title compound. MS (ESI pos. ion) m/z: 504 (MH+). ¹H NMR (300 MHz, DMSO-d₆) δ: 8.88 (s, 1H), 8.20 (d, J=4.5 Hz, 1H), 8.13 (dd, J=4.1, 1.8 Hz, 1H), 7.69 (s, 1H), 7.65 (d, J=8.0 Hz, 1H), 7.42-7.57 (m, 2H), 7.20-7.39 (m, 4H), 4.27-4.41 (m, 1H), 4.00-4.20 (m, 1H), 3.19-3.29 (m, 1H), 2.82-2.96 (m, 1H), 2.75 (d, J=4.5 Hz, 3H), 2.35 (s, 3H).

### Example 297

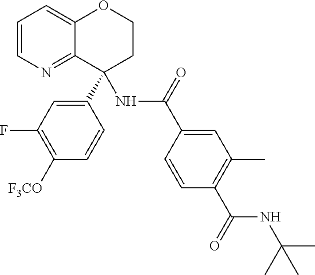 [SMILES: Cc1cc(C(=O)N[C@]2(c3ccc(OC(F)(F)F)c(F)c3)CCOc3cccnc32)ccc1C(=O)NC(C)(C)C]

### (S)—N1-(tert-butyl)-N4-(4-(3-fluoro-4-(trifluoromethoxy)phenyl)-3,4-dihydro-2H-pyrano[3,2-b]pyridin-4-yl)-2-methylterephthalamide

The title compound was prepared using the same method as described in Example 296 using 2-methylpropan-2-amine. MS (ESI pos. ion) m/z: 546 (MH+). ¹H NMR (300 MHz, DMSO-d₆) δ: 8.85 (s, 1H), 8.12 (dd, J=4.0, 1.8 Hz, 1H), 7.85 (s, 1H), 7.68 (s, 1H), 7.59-7.67 (m, 1H), 7.42-7.56 (m, 2H), 7.19-7.34 (m, 4H), 4.33 (ddd, J=11.3, 8.1, 2.7 Hz, 1H), 4.05-4.21 (m, 1H), 3.19-3.29 (m, 1H), 2.81-2.96 (m, 1H), 2.32 (s, 3H), 1.36 (s, 9H).

### Example 298

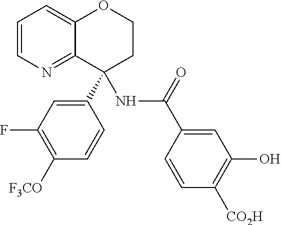 [SMILES: O=C(N[C@]1(c2ccc(OC(F)(F)F)c(F)c2)CCOc2cccnc21)c1ccc(C(=O)O)c(O)c1]

### (S)-4-((4-(3-fluoro-4-(trifluoromethoxy)phenyl)-3,4-dihydro-2H-pyrano[3,2-b]pyridin-4-yl)carbamoyl)-2-hydroxybenzoic acid

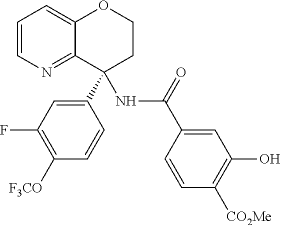 [SMILES: COC(=O)c1ccc(C(=O)N[C@]2(c3ccc(OC(F)(F)F)c(F)c3)CCOc3cccnc32)cc1O]

Step 1: (S)-methyl 4-((4-(3-fluoro-4-(trifluoromethoxy)phenyl)-3,4-dihydro-2H-pyrano[3,2-b]pyridin-4-yl)carbamoyl)-2-hydroxybenzoate

To a stirred solution of (S)-methyl 2-amino-4-((4-(3-fluoro-4-(trifluoromethoxy)phenyl)-3,4-dihydro-2H-pyrano[3,2-b]pyridin-4-yl)carbamoyl)benzoate (0.274 g, 0.542 mmol) in DCE (5 mL) was added tert-butyl nitrite, 90% (0.090 ml, 0.759 mmol) dropwise. After the addition, the mixture was stirred for 1 h. The mixture was then cooled in an ice bath and 1.0 mL of concentrated H₂SO₄ was added followed by 1 mL of H₂O. 2 mL of MeCN was added, and the mixture was then heated at reflux for 24 h. The reaction mixture was then cooled, concentrated, taken in H₂O, neutralized with saturated NaHCO₃, and extracted with DCM (3×). The extracts were dried over Na₂SO₄, concentrated, and purified by flash column chromatography (0-50% EtOAc in hexanes) to give the title compound. MS (ESI pos. ion) m/z: 507 (MH+).

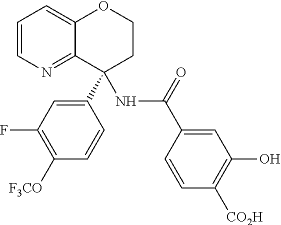 [SMILES: O=C(N[C@]1(c2ccc(OC(F)(F)F)c(F)c2)CCOc2cccnc21)c1ccc(C(=O)O)c(O)c1]

Step 2: (S)-4-((4-(3-fluoro-4-(trifluoromethoxy)phenyl)-3,4-dihydro-2H-pyrano[3,2-b]pyridin-4-yl)carbamoyl)-2-hydroxybenzoic acid

(S)-methyl 4-((4-(3-fluoro-4-(trifluoromethoxy)phenyl)-3,4-dihydro-2H-pyrano[3,2-b]pyridin-4-yl)carbamoyl)-2-hydroxybenzoate (75 mg, 0.148 mmol) was dissolved in MeOH (5 mL), and 5N NaOH (1 mL) was added. The mixture was stirred at room temperature overnight. The mixture was then concentrated and acidified with 5N HCl. The solid was collected and purified by reverse phase HPLC. The pure fractions were concentrated to minimal H₂O and adjusted to pH=3 with saturated aqueous NaHCO₃. The solid was filtered, washed with H₂O, and dried to give the title product. MS (ESI pos. ion) m/z: 493 (MH+). ¹H NMR (400 MHz, DMSO-d₆) δ: 11.27 (br. s., 1H), 8.99 (s, 1H), 8.12 (br. s., 1H), 7.82 (d, J=8.0 Hz, 1H), 7.41-7.60 (m, 2H), 7.37 (s, 1H), 7.19-7.35 (m, 4H), 4.33 (t, J=8.6 Hz, 1H), 4.12 (t, J=8.9 Hz, 1H), 3.20-3.25 (m, 1H), 2.76-2.92 (m, 1H).

### Example 299

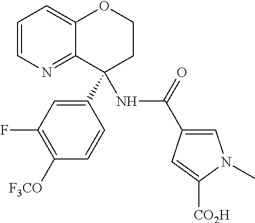 [SMILES: Cn1cc(C(=O)N[C@]2(c3ccc(OC(F)(F)F)c(F)c3)CCOc3cccnc32)cc1C(=O)O]

### (S)-4-((4-(3-fluoro-4-(trifluoromethoxy)phenyl)-3,4-dihydro-2H-pyrano[3,2-b]pyridin-4-yl)carbamoyl)-1-methyl-1H-pyrrole-2-carboxylic acid

To a stirred mixture of (S)-ethyl 4-((4-(3-fluoro-4-(trifluoromethoxy)phenyl)-3,4-dihydro-2H-pyrano[3,2-b]pyridin-4-yl)carbamoyl)-1H-pyrrole-2-carboxylate (0.240 g, 0.486 mmol) and K₂CO₃ (0.168 g, 1.216 mmol) in DMF (5 mL) was added iodomethane (0.046 ml, 0.730 mmol). The reaction mixture was stirred at room temperature for 2 h, H₂O was added, and the solution was extracted with EtOAc (3×). The combined organic layers were dried over Na₂SO₄, concentrated and purified by flash column chromatography (0-50% EtOAc in hexanes). The material was dissolved in MeOH (5 mL), and 5N NaOH (0.250 mL) was added. The reaction was stirred at room temperature overnight, concentrated, and acidified with 5N HCl. The white solid was collected, washed with H₂O, and dried to give the title compound. MS (ESI pos. ion) m/z: 480 (MH+). ¹H NMR (400 MHz, DMSO-d₆) δ: 12.43 (br. s., 1H), 8.33 (s, 1H), 8.09 (br. s., 1H), 7.59 (s, 1H), 7.39-7.52 (m, 2H), 7.32 (s, 1H), 7.22-7.30 (m, 2H), 7.19 (d, J=8.6 Hz, 1H), 4.24-4.36 (m, 1H), 4.15 (br. s., 1H), 3.84 (s, 3H), 3.24 (dd, J=14.0, 5.8 Hz, 1H), 2.75-2.89 (m, 1H).

### Example 300

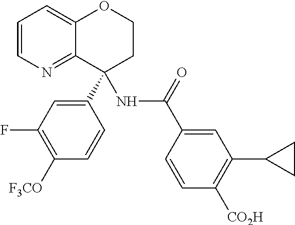 [SMILES: O=C(N[C@]1(c2ccc(OC(F)(F)F)c(F)c2)CCOc2cccnc21)c1ccc(C(=O)O)c(C2CC2)c1]

### (S)-2-cyclopropyl-4-((4-(3-fluoro-4-(trifluoromethoxy)phenyl)-3,4-dihydro-2H-pyrano[3,2-b]pyridin-4-yl)carbamoyl)benzoic acid

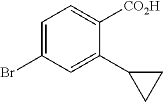 [SMILES: O=C(O)c1ccc(Br)cc1C1CC1]

Step 1: 4-bromo-2-cyclopropylbenzoic acid

To a stirred solution of 4-bromo-2-fluorobenzoic acid (0.75 g, 3.42 mmol) in THF (7 mL) at 0° C. was added cyclopropylmagnesium bromide (20.5 mL, 10.27 mmol) dropwise. After the addition, the mixture was stirred at 0° C. for 3 h, quenched with 5N HCl, extracted with EtOAc (3×). The extracts were dried over Na₂SO₄, concentrated, purified by flash column chromatography (0-15% MeOH in DCM) to give the title compound. MS (ESI pos. ion) m/z: 242.0 (MH+).

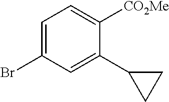 [SMILES: COC(=O)c1ccc(Br)cc1C1CC1]

Step 2: methyl 4-bromo-2-cyclopropylbenzoate

4-bromo-2-cyclopropylbenzoic acid (0.75 g, 0.726 mmol) was dissolved in DMF (5 mL), and potassium carbonate (0.207 ml, 3.42 mmol) was added, followed by iodomethane (2.430 g, 17.12 mmol). After stirring at room temperature for 2 h, H₂O was added, and the reaction was extracted with DCM (3×). The extracts were dried over Na₂SO₄, concentrated to give the title compound. MS (ESI pos. ion) m/z: 256.0 (MH+).

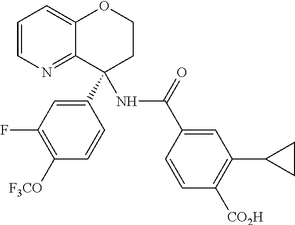 [SMILES: O=C(N[C@]1(c2ccc(OC(F)(F)F)c(F)c2)CCOc2cccnc21)c1ccc(C(=O)O)c(C2CC2)c1]

Step 3: (S)-2-cyclopropyl-4-((4-(3-fluoro-4-(trifluoromethoxy)phenyl)-3,4-dihydro-2H-pyrano[3,2-b]pyridin-4-yl)carbamoyl)benzoic acid

A mixture of methyl 4-bromo-2-cyclopropylbenzoate (0.155 g, 0.609 mmol), (S)-4-(3-fluoro-4-(trifluoromethoxy)phenyl)-3,4-dihydro-2H-pyrano[3,2-b]pyridin-4-amine (0.200 g, 0.609 mmol), xantphos (0.035 g, 0.061 mmol), palladium (II) acetate (0.014 g, 0.061 mmol), and TEA (0.255 ml, 1.828 mmol) in DMF (5 ml) was degassed. CO gas was bubbled through for 5 min, and the reaction mixture was then heated at 80° C. for 16 h. The reaction mixture was then cooled, concentrated to dryness, and purified by flash column chromatography (0-50% EtOAc in hexanes). The resulting material was dissolved in MeOH (3 mL) and added 1 mL of 5N NaOH and stirred for 36 h. The mixture was concentrated, acidified with 5N HCl. The white solid was filtered, washed with H₂O, dried to give the title compound. MS (ESI pos. ion) m/z: 517.0 (MH+). ¹H NMR (400 MHz, DMSO-d₆) δ: 13.11 (br. s., 1H), 9.00 (br. s., 1H), 8.12 (br. s., 1H), 7.71 (d, J=7.6 Hz, 1H), 7.64 (d, J=7.8 Hz, 1H), 7.43-7.57 (m, 2H), 7.26-7.37 (m, 3H), 7.22 (d, J=8.4 Hz, 1H), 4.24-4.40 (m, 1H), 4.11 (t, J=8.7 Hz, 1H), 3.11-3.28 (m, 1H), 2.75-2.92 (m, 1H), 2.62 (br. s., 1H), 0.97 (d, J=7.6 Hz, 2H), 0.74 (br. s., 2H).

### Example 301

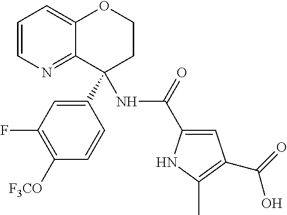 [SMILES: Cc1[nH]c(C(=O)N[C@]2(c3ccc(OC(F)(F)F)c(F)c3)CCOc3cccnc32)cc1C(=O)O]

### (S)-5-(4-(3-fluoro-4-(trifluoromethoxy)phenyl)-3,4-dihydro-2H-pyrano[3,2-b]pyridin-4-yl)carbamoyl)-2-methyl-1H-pyrrole-3-carboxylic acid

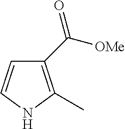

Step 1: Methyl 2-methyl-1H-pyrrole-3-carboxylate

A mixture of methyl 3-oxobutanoate (1.0 g, 8.61 mmol), acetic acid, ammonia salt (1.328 g, 17.22 mmol), chloroacetaldehyde (50% in water, 1.330 ml, 10.33 mmol), and ammonia (2.0M in EtOH, 12.92 ml, 25.8 mmol) in a sealed tube was heated at 50° C. for 2 h. The reaction mixture was cooled, taken up in H₂O, extracted with DCM (3×). The extracts were dried over Na₂SO₄, concentrated, and purified by flash column chromatography (0-60% EtOAc in hexanes) to give the title compound. MS (ESI pos. ion) m/z: 140.0 (MH+). ¹H NMR (400 MHz, DMSO-d₆) δ: 11.21 (br. s., 1H), 6.60 (t, J=2.6 Hz, 1H), 6.31 (t, J=2.7 Hz, 1H), 3.65-3.69 (s, 3H), 2.40 (s, 3H).

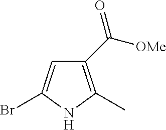

Step 2: Methyl 5-bromo-2-methyl-1H-pyrrole-3-carboxylate

To a stirred solution of methyl 2-methyl-1H-pyrrole-3-carboxylate (1.16 g, 8.34 mmol) in THF (20 mL) at −78° C. was added NBS (1.484 g, 8.34 mmol). The reaction mixture was stirred at −78° C. for 1 h, quenched with saturated aqueous NaHCO₃, extracted with DCM (3×). The extracts were dried over Na₂SO₄, concentrated to dryness, triturated in EtOAc, and the filtrate concentrated and used in the next step without further purification. MS (ESI pos. ion) m/z: 219.0 (MH+).

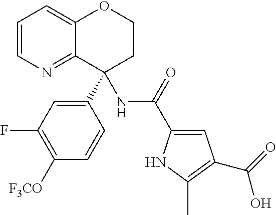 [SMILES: Cc1[nH]c(C(=O)N[C@]2(c3ccc(OC(F)(F)F)c(F)c3)CCOc3cccnc32)cc1C(=O)O]

Step 3: (S)-5-(4-(3-fluoro-4-(trifluoromethoxy)phenyl)-3,4-dihydro-2H-pyrano[3,2-b]pyridin-4-yl)carbamoyl)-2-methyl-1H-pyrrole-3-carboxylic acid

The title compound was prepared in the same method as described in Example 300 step 3 using methyl 5-bromo-2-methyl-1H-pyrrole-3-carboxylate. MS (ESI pos. ion) m/z: 480 (MH+). ¹H NMR (400 MHz, DMSO-d₆) δ: 11.82 (br. s., 1H), 11.73 (br. s., 1H), 8.47 (s, 1H), 8.11 (d, J=3.1 Hz, 1H), 7.41-7.59 (m, 2H), 7.09-7.35 (m, 4H), 4.31 (t, J=8.9 Hz, 1H), 4.06-4.19 (m, 1H), 3.24 (dd, J=13.4, 5.4 Hz, 1H), 2.78-2.88 (m, 1H), 2.38 (s, 3H).

### Example 302

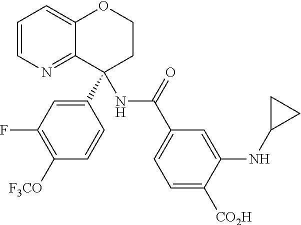 [SMILES: O=C(N[C@]1(c2ccc(OC(F)(F)F)c(F)c2)CCOc2cccnc21)c1ccc(C(=O)O)c(NC2CC2)c1]

### (S)-2-(cyclopropylamino)-4-((4-(3-fluoro-4-(trifluoromethoxy)phenyl)-3,4-dihydro-2H-pyrano[3,2-b]pyridin-4-yl)carbamoyl)benzoic acid

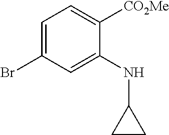 [SMILES: COC(=O)c1ccc(Br)cc1NC1CC1]

Step 1: Methyl 4-bromo-2-(cyclopropylamino)benzoate

A mixture of 4-bromo-2-fluorobenzoic acid (1.254 g, 5.73 mmol) and cyclopropylamine (2.69 g, 28.6 mmol) in pyridine (5 mL) was heated at 100° C. for 48 h. The reaction mixture was cooled, concentrated to dryness, and dissolved in DMF (10 mL). K₂CO₃ (0.726 g, 5.25 mmol) and iodomethane (1.069 ml, 17.18 mmol) were then added. The mixture was then stirred at room temperature overnight. H₂O was added and the mixture was extracted with EtOAc (3×). The extracts were dried over Na₂SO₄, concentrated and purified by flash column chromatography (0-10% EtOAc in hexanes) to give the title compound. MS (ESI pos. ion) m/z: 245 (MH+).

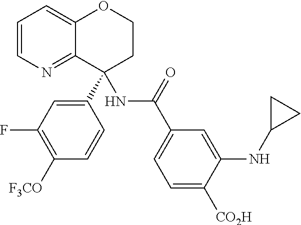 [SMILES: O=C(N[C@]1(c2ccc(OC(F)(F)F)c(F)c2)CCOc2cccnc21)c1ccc(C(=O)O)c(NC2CC2)c1]

Step 2: (S)-2-(cyclopropylamino)-4-((4-(3-fluoro-4-(trifluoromethoxy)phenyl)-3,4-dihydro-2H-pyrano[3,2-b]pyridin-4-yl)carbamoyl)benzoic acid

The title compound was prepared in the same method as described in Example 300 step 3 using methyl 4-bromo-2-(cyclopropylamino)benzoate. MS (ESI pos. ion) m/z: 532.0 (MH+). ¹H NMR (400 MHz, DMSO-d₆) δ: 8.88 (s, 1H), 8.16 (dd, J=4.0, 1.9 Hz, 1H), 8.02 (br. s., 1H), 7.81 (d, J=8.2 Hz, 1H), 7.46-7.57 (m, 2H), 7.44 (d, J=1.4 Hz, 1H), 7.26-7.36 (m, 2H), 7.20-7.26 (m, 1H), 7.05 (dd, J=8.2, 1.6 Hz, 1H), 4.30-4.41 (m, 1H), 4.12 (ddd, J=11.3, 8.5, 2.5 Hz, 1H), 3.18 (ddd, J=14.4, 8.3, 2.9 Hz, 2H), 2.98 (ddd, J=14.2, 7.1, 2.7 Hz, 1H), 0.71-0.83 (m, 2H), 0.41-0.50 (m, 2H).

### Example 303

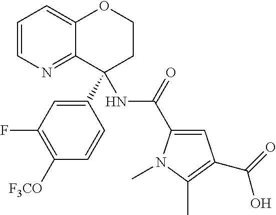 [SMILES: Cc1c(C(=O)O)cc(C(=O)N[C@]2(c3ccc(OC(F)(F)F)c(F)c3)CCOc3cccnc32)n1C]

### (S)-5-(4-(3-fluoro-4-(trifluoromethoxy)phenyl)-3,4-dihydro-2H-pyrano[3,2-b]pyridin-4-yl)carbamoyl)-1,2-dimethyl-1H-pyrrole-3-carboxylic acid

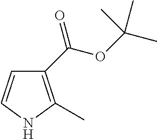

Step 1: tert-Butyl 2-methyl-1H-pyrrole-3-carboxylate

A mixture of tert-butyl acetoacetate (1 g, 6.32 mmol), chloroacetaldehyde (50% solution in water, 0.976 ml, 7.59 mmol), ammonium acetate (1.362 ml, 18.96 mmol), and ammonia (2.0M in EtOH, 6.32 ml, 12.64 mmol) was heated at 60° C. for 2 h. The reaction mixture was then cooled, H₂O was added, and the solution was extracted with EtOAc (3×). The combined organic layers were dried over MgSO₄, concentrated, purified by flash column chromatography (0-40% EtOAc in hexanes) to give the title compound as an orange solid. MS (ESI pos. ion) m/z: 182 (MH+).

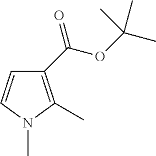

Step 2: tert-butyl 1,2-dimethyl-1H-pyrrole-3-carboxylate

A mixture of tert-butyl 2-methyl-1H-pyrrole-3-carboxylate (1.54 g, 8.50 mmol), Cs₂CO₃ (4.15 g, 12.75 mmol), and iodomethane (0.793 ml, 12.75 mmol) in DMF (20 mL) was stirred at room temperature for 24 h. H₂O was added, and the aqueous solution was extracted with EtOAc (3×). The combined organic layers were dried over Na₂SO₄ and concentrated to give the title compound as a tan solid. MS (ESI pos. ion) m/z: 196.0 (MH+).

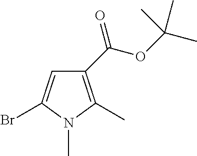

Step 3: tert-butyl 5-bromo-1,2-dimethyl-1H-pyrrole-3-carboxylate

The title compound was prepared using the same method as described in Example 301 Step 2 using tert-butyl 1,2-dimethyl-1H-pyrrole-3-carboxylate. MS (ESI pos. ion) m/z: 274 (MH+).

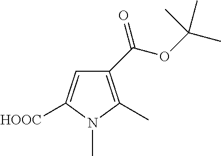 [SMILES: Cc1c(C(=O)OC(C)(C)C)cc(C(=O)O)n1C]

Step 4: 4-(tert-butoxycarbonyl)-1,5-dimethyl-1H-pyrrole-2-carboxylic acid

A mixture of tert-butyl 5-bromo-1,2-dimethyl-1H-pyrrole-3-carboxylate (1.64 g, 5.98 mmol), (9,9-dimethyl-9H-xanthene-4,5-diyl)bis(diphenylphosphine) (0.173 g, 0.299 mmol), triethylamine (2.501 ml, 17.95 mmol), and palladium (II) acetate (0.134 g, 0.598 mmol) in THF/H₂O (2:1, 15 mL) was degassed. CO gas was bubbled through for 15 min, and the mixture was then heated at 60° C. for 16 h under a CO balloon. The reaction was then heated at 80° C. for another 24 h. The mixture was then cooled, diluted with H₂O, and extracted with Et₂O (discarded). The aqueous layer was cooled in an ice bath and acidified with 2N HCl. The title compound was collected as a tan solid, dried, and used in the next step. MS (ESI pos. ion) m/z: 240 (MH+).

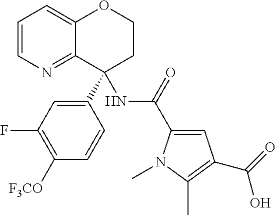 [SMILES: Cc1c(C(=O)O)cc(C(=O)N[C@]2(c3ccc(OC(F)(F)F)c(F)c3)CCOc3cccnc32)n1C]

Step 5: (S)-5-(4-(3-fluoro-4-(trifluoromethoxy)phenyl)-3,4-dihydro-2H-pyrano[3,2-b]pyridin-4-yl)carbamoyl)-1,2-dimethyl-1H-pyrrole-3-carboxylic acid

To a stirred mixture of 4-(tert-butoxycarbonyl)-1,5-dimethyl-1H-pyrrole-2-carboxylic acid (0.040 g, 0.167 mmol), (S)-4-(3-fluoro-4-(trifluoromethoxy)-phenyl)-3,4-dihydro-2H-pyrano[3,2-b]pyridin-4-amine (0.060 g, 0.184 mmol), and DIPEA (0.038 ml, 0.217 mmol) in DMF (2 mL) was added HATU (0.076 g, 0.201 mmol). The mixture was stirred for 16 h and then heated at 60° C. for 1 h. The mixture was cooled, H₂O was added, and the solid was collected. The solid was dried and purified by flash chromatography (0-50% EtOAc in hexanes). The resulting ester was dissolved in DCM, and TFA (1 mL) was added, and the mixture was stirred for 16 h. The mixture was then concentrated, taken up in H₂O, and neutralized by saturated aqueous NaHCO₃. The tan solid was collected and dried to give the title compound. MS (ESI pos. ion) m/z: 494 (MH+). ¹H NMR (400 MHz, DMSO-d₆) δ: 8.56 (s, 1H), 8.12 (dd, J=4.3, 1.8 Hz, 1H), 7.43-7.53 (m, 2H), 7.21-7.35 (m, 4H), 4.34 (ddd, J=11.2, 8.6, 2.6 Hz, 1H), 4.09-4.22 (m, 1H), 3.63-3.71 (m, 3H), 3.08-3.21 (m, 1H), 2.85 (ddd, J=14.1, 8.5, 2.8 Hz, 1H), 2.46 (s, 3H).

### Example 304

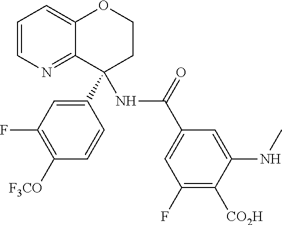 [SMILES: CNc1cc(C(=O)N[C@]2(c3ccc(OC(F)(F)F)c(F)c3)CCOc3cccnc32)cc(F)c1C(=O)O]

### (S)-2-fluoro-4-((4-(3-fluoro-4-(trifluoromethoxy)phenyl)-3,4-dihydro-2H-pyrano[3,2-b]pyridin-4-yl)carbamoyl)-6-(methylamino)benzoic acid

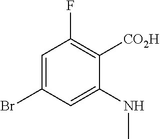

Step 1: 4-bromo-2-fluoro-6-(methylamino)benzoic acid

A mixture of 4-bromo-2,6-difluorobenzoic acid (1.0 g, 4.22 mmol), DMAP (0.077 g, 0.633 mmol), and methylamine (1.191 g, 12.66 mmol) in pyridine (10 mL) was heated at 100° C. for 16 h. The reaction mixture was then cooled, diluted with EtOAc, washed with 5N HCl, H₂O, brine, dried over Na₂SO₄, and concentrated to give the title compound as a light yellow solid. MS (ESI pos. ion) m/z: 248 (MH+).

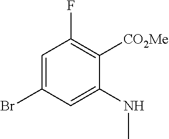

Step 2: Methyl 4-bromo-2-fluoro-6-(methylamino)benzoate

The title compound was prepared in the same method as described in Example 300 step 2 using 4-bromo-2-fluoro-6-(methylamino)benzoic acid. MS (ESI pos. ion) m/z: 262 (MH+). ¹H NMR (400 MHz, CDCl₃) δ: 7.72 (br. s., 1H), 6.57 (s, 1H), 6.50 (dd, J=11.2, 1.8 Hz, 1H), 3.87 (s, 3H), 2.87 (d, J=5.1 Hz, 3H).

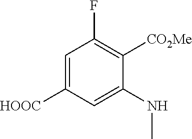 [SMILES: CNc1cc(C(=O)O)cc(F)c1C(=O)OC]

Step 3: 3-Fluoro-4-(methoxycarbonyl)-5-(methylamino)benzoic acid

The title compound was prepared in the same method as described in Example 303 step 4 using methyl 4-bromo-2-fluoro-6-(methylamino)benzoate.

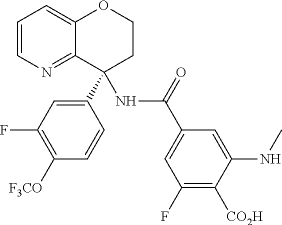 [SMILES: CNc1cc(C(=O)N[C@]2(c3ccc(OC(F)(F)F)c(F)c3)CCOc3cccnc32)cc(F)c1C(=O)O]

Step 4: (S)-2-fluoro-4-((4-(3-fluoro-4-(trifluoromethoxy)phenyl)-3,4-dihydro-2H-pyrano[3,2-b]pyridin-4-yl)carbamoyl)-6-(methylamino)benzoic acid

The title compound was prepared in the same method as described in Example 287 using 3-fluoro-4-(methoxycarbonyl)-5-(methylamino)benzoic acid. MS (ESI pos. ion) m/z: 524 (MH+). ¹H NMR (400 MHz, DMSO-d₆) δ: 8.94 (s, 1H), 8.13 (dd, J=4.2, 1.7 Hz, 1H), 7.42-7.56 (m, 2H), 7.24-7.34 (m, 2H), 7.18-7.24 (m, 1H), 6.85 (s, 1H), 6.81-6.84 (m, 1H), 4.32 (ddd, J=11.2, 7.9, 2.8 Hz, 1H), 4.12 (ddd, J=11.2, 8.0, 2.7 Hz, 1H), 3.25 (ddd, J=14.2, 7.7, 2.7 Hz, 1H), 2.84 (s, 3H), 2.76-2.91 (ddd, J=14.2, 7.7, 2.7 Hz, 1H).

### Example 305

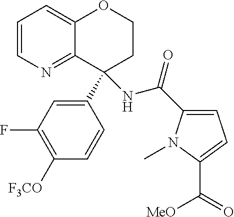 [SMILES: COC(=O)c1ccc(C(=O)N[C@]2(c3ccc(OC(F)(F)F)c(F)c3)CCOc3cccnc32)n1C]

### (S)-methyl 5-((4-(3-fluoro-4-(trifluoromethoxy)phenyl)-3,4-dihydro-2H-pyrano[3,2-b]pyridin-4-yl)carbamoyl)-1-methyl-1H-pyrrole-2-carboxylate

The title compound was prepared in the same method as described in Example 300 step 3 using methyl 5-bromo-1-methyl-1H-pyrrole-2-carboxylate. MS (ESI pos. ion) m/z: 494 (MH+). ¹H NMR (400 MHz, DMSO-d₆) δ: 8.38 (s, 1H), 8.09 (dd, J=4.2, 1.7 Hz, 1H), 7.63 (d, J=1.8 Hz, 1H), 7.42-7.49 (m, 2H), 7.41 (d, J=2.0 Hz, 1H), 7.22-7.30 (m, 2H), 7.19 (dd, J=8.8, 1.2 Hz, 1H), 4.29 (ddd, J=11.3, 8.7, 2.5 Hz, 1H), 4.10-4.20 (m, 1H), 3.85 (s. 3H), 3.74 (s, 3H), 3.18-3.30 (m, 1H), 2.80 (ddd, J=14.2, 8.7, 3.1 Hz, 1H).

### Example 306

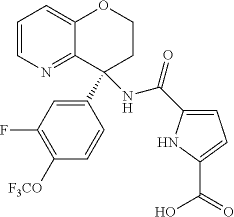 [SMILES: O=C(O)c1ccc(C(=O)N[C@]2(c3ccc(OC(F)(F)F)c(F)c3)CCOc3cccnc32)[nH]1]

### (S)-5-((4-(3-fluoro-4-(trifluoromethoxy)phenyl)-3,4-dihydro-2H-pyrano[3,2-b]pyridin-4-yl)carbamoyl)-1H-pyrrole-2-carboxylic acid

The title compound was prepared in the same method as described in Example 287 using 5-(methoxycarbonyl)-1H-pyrrole-2-carboxylic acid. MS (ESI pos. ion) m/z: 466 (MH+). ¹H NMR (400 MHz, DMSO-d₆) δ: 12.75 (br. s., 1H), 12.25 (br. s., 1H), 8.69-8.78 (m, 1H), 8.13 (dd, J=4.3, 1.6 Hz, 1H), 7.44-7.56 (m, 2H), 7.24-7.36 (m, 2H), 7.14-7.24 (m, 1H), 6.66-6.75 (m, 2H), 4.30 (ddd, J=11.2, 8.1, 2.6 Hz, 1H), 4.05-4.16 (m, 1H), 3.29-3.33 (m, 1H), 2.70-2.81 (m, 1H).

### Example 307

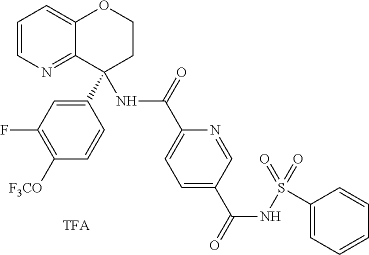 [SMILES: O=C(NS(=O)(=O)c1ccccc1)c1ccc(C(=O)N[C@]2(c3ccc(OC(F)(F)F)c(F)c3)CCOc3cccnc32)nc1]

### (S)—N2-(4-(3-fluoro-4-(trifluoromethoxy)phenyl)-3,4-dihydro-2H-pyrano[3,2-b]pyridin-4-yl)-N5-(phenylsulfonyl)pyridine-2,5-dicarboxamide 2,2,2-trifluoroacetate

To a solution of (S)-6-((4-(3-fluoro-4-(trifluoromethoxy)-phenyl)-3,4-dihydro-2H-pyrano[3,2-b]pyridin-4-yl)carbamoyl)nicotinic acid (46 mg, 0.096 mmol) and DMF (2 mL), were 4 added benzenesulfonamide (18.18 mg, 0.116 mmol), HATU (44.0 mg, 0.116 mmol), and DIPEA (0.042 mL, 0.241 mmol). The solution was stirred at room temperature. After 18 h, the reaction was treated with more HATU (30 mg). The reaction mixture was purified by reverse-phase preparative HPLC on a Phenomenex Luna column (5 micron, Phenyl-hexyl, 100 Å, 100×30 mm) eluting at 45 mL/min with a linear gradient of 30% to 60% MeCN (0.1% TFA) in water (0.1% TFA) to give the title compound as a white solid after lypholization. MS (ESI pos. ion) m/z: 617.0 (MH+). ¹H NMR (300 MHz, MeOH-d₄) δ: 8.97 (s, 1H), 8.31 (dd, J=8.2, 2.2 Hz, 1H), 8.26 (dd, J=3.8, 2.2 Hz, 1H), 8.07-8.19 (m, 3H), 7.66-7.76 (m, 1H), 7.56-7.66 (m, 2H), 7.34-7.48 (m, 4H), 7.18-7.28 (m, 1H), 4.42 (dt, J=12.1, 4.3 Hz, 1H), 4.09 (td, J=11.4, 2.5 Hz, 1H), 3.25 (dd, J=4.7, 2.5 Hz, 1H), 3.02-3.16 (m, 1H).

### Example 308

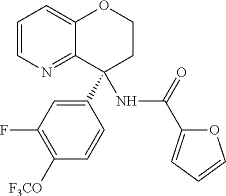 [SMILES: O=C(N[C@]1(c2ccc(OC(F)(F)F)c(F)c2)CCOc2cccnc21)c1ccco1]

### (S)—N-(4-(3-fluoro-4-(trifluoromethoxy)phenyl)-3,4-dihydro-2H-pyrano[3,2-b]pyridin-4-yl)furan-2-carboxamide

To a RBF were added (S)-5-bromo-N-(4-(3-fluoro-4-(trifluoromethoxy)phenyl)-3,4-dihydro-2H-pyrano[3,2-b]pyridin-4-yl)furan-2-carboxamide (100 mg, 0.200 mmol), K₂CO₃ (119.8 mg, 0.867 mmol), (9,9-dimethyl-9H-xanthene-4,5-diyl)bis(diphenylphosphine) (23.2 mg, 0.040 mmol), and bis(tri-tertbutylphosphine)palladium (0) (40.8 mg, 0.080 mmol). The RBF was cycled (3×) between vacuum and N₂. The solids were then suspended in DMF (2 mL) and water (36 μL, 1.998 mmol). CO gas was bubbled through the solution for 30 sec and then the reaction was heated in a 80° C. oil bath and stirred under an atmosphere of CO (balloon). After 22 h, the reaction was allowed to cool to room temperature and diluted with water (30 mL). The aqueous solution was extracted with EtOAc (3×10 mL). The combined EtOAc layers were concentrated in vacuo and adsorbed onto a plug of silica gel and chromatographed through a Redi-Sep® pre-packed silica gel column (4 g), eluting with 0-100% EtOAc in hexanes, to afford the title compound as a white solid. MS (ESI pos. ion) m/z: 423.0 (MH+). ¹H NMR (300 MHz, MeOH—d₄) δ: 8.23 (t, J=3.0 Hz, 1H), 7.65 (dd, J=1.8, 0.7 Hz, 1H), 7.36-7.47 (m, 2H), 7.34 (d, J=2.9 Hz, 2H), 7.18-7.26 (m, 1H), 7.12 (dd, J=3.5, 0.7 Hz, 1H), 6.58 (dd, J=3.5, 1.8 Hz, 1H), 4.40 (dt, J=11.9, 4.1 Hz, 1H), 3.97-4.11 (m, 1H), 3.19-3.29 (m, 1H), 3.01-3.16 (m, 1H).

### Example 309

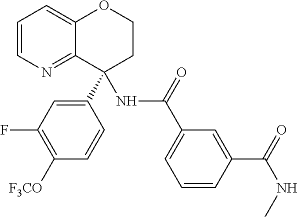 [SMILES: CNC(=O)c1cccc(C(=O)N[C@]2(c3ccc(OC(F)(F)F)c(F)c3)CCOc3cccnc32)c1]

### (S)—N1-(4-(3-fluoro-4-(trifluoromethoxy)phenyl)-3,4-dihydro-2H-pyrano[3,2-b]pyridin-4-yl)-N3-methylisophthalamide

To a RBF containing (S)-3-((4-(3-fluoro-4-(trifluoromethoxy)phenyl)-3,4-dihydro-2H-pyrano[3,2-b]pyridin-4-yl)carbamoyl)benzoic acid (130 mg, 0.273 mmol) and EtOAc (3 mL) was added methylamine (33% in EtOH, 2 mL, 16.07 mmol) and 50% T3P in EtOAc (0.6 mL, 0.713 mmol). The reaction was stirred at room temperature. After 18 h, LC-MS showed only ˜10-20% conversion. The reaction was treated with more methylamine (2 mL) and T3P (0.6 mL). After a further 24 h, the reaction was partitioned between EtOAc and water (1:1, 10 mL each). The aqueous layer was extracted with EtOAc (5 mL). The combined EtOAc layers were concentrated in vacuo and adsorbed onto a plug of silica gel and chromatographed through a Redi-Sep® pre-packed silica gel column (4 g), eluting with 0-50% EtOAc in hexanes, to afford the title compound as a white solid. MS (ESI pos. ion) m/z: 490.1 (MH+). ¹H NMR (300 MHz, MeOH-d₄) δ: 8.23 (t, J=1.6 Hz, 1H), 8.19 (dd, J=3.9, 2.0 Hz, 1H), 7.90-8.01 (m, 2H), 7.55 (t, J=7.7 Hz, 1H), 7.28-7.49 (m, 4H), 7.18-7.28 (m, 1H), 4.35-4.47 (m, 1H), 4.02-4.16 (m, 1H), 3.33-3.40 (m, 1H), 2.98-3.10 (m, 1H), 2.92 (s, 3H)

### Intermediate 48

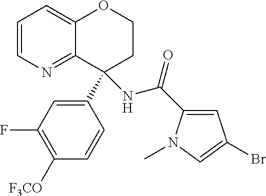 [SMILES: Cn1cc(Br)cc1C(=O)N[C@]1(c2ccc(OC(F)(F)F)c(F)c2)CCOc2cccnc21]

### (S)-4-bromo-N-(4-(3-fluoro-4-(trifluoromethoxy)phenyl)-3,4-dihydro-2H-pyrano[3,2-b]pyridin-4-yl)-1-methyl-1H-pyrrole-2-carboxamide

A mixture of (S)-4-bromo-N-(4-(3-fluoro-4-(trifluoromethoxy)phenyl)-3,4-dihydro-2H-pyrano[3,2-b]pyridin-4-yl)-1H-pyrrole-2-carboxamide (0.350 g, 0.700 mmol), Cs₂CO₃ (0.342 g, 1.049 mmol), and iodomethane (0.066 ml, 1.049 mmol) in DMF (10 mL) was stirred at room temperature for 24 h. H₂O was added and the aqueous layer was extracted with EtOAc (3×). The combined organic layers were dried over MgSO₄ and concentrated to give the title compound. MS (ESI pos. ion) m/z: 514 (MH+).

### General Procedure for Ester Hydrolysis

A mixture of ester (1 eq) and LiOH or NaOH in THF:MeOH (3:1,), unless otherwise specified, was stirred at a temperature and time as indicated in the appendix to Table 15. The reaction was then purified by one of the following methods: Method A: The reaction was cooled, concentrated, diluted with H₂O, acidified to pH 4 with 5N HCl. The resulting solid was collected, washed with H₂O, and dried to give the title compound; Method B: The reaction was neutralized with 1N HCl in H₂O, diluted with H₂O, and extracted with EtOAc (3×25 mL). The organic layers were combined, washed with brine, dried (MgSO₄), and concentrated in vacuo to give the title compound; and Method C: The reaction was filtered through a syringe filter, and then purified by preparative reverse phase HPLC (gradient elution 10-100% MeCN/0.1% TFA in H₂O). The product-containing fractions were then combined and the solvent was removed by lyophilization to provide the target compound as the TFA salt.

TABLE 15
Examples 310-313 and 316-330 prepared via ester hydrolysis
analogous to general procedures described above.

| | Ester | Product | Product | MS |
|---|---|---|---|---|
| Ex # | Intermediate | Structure | Name | M + H |
| 310^{a} | 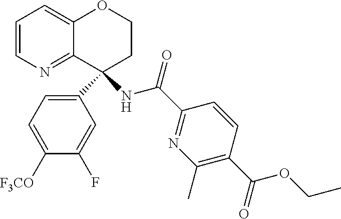 [SMILES: CCOC(=O)c1ccc(C(=O)N[C@]2(c3ccc(OC(F)(F)F)c(F)c3)CCOc3cccnc32)nc1C] | 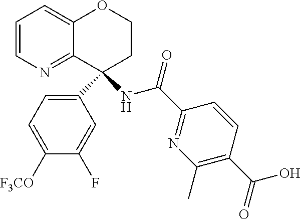 [SMILES: Cc1nc(C(=O)N[C@]2(c3ccc(OC(F)(F)F)c(F)c3)CCOc3cccnc32)ccc1C(=O)O] | (S)-6-((4-(3- fluoro-4- (trifluoro- methoxy) phenyl)-3,4- dihydro-2H- pyrano[3,2-b] pyridin-4-yl) carbamoyl)-2- methylnicotinic acid | 491.9 |
| 311^{b} | 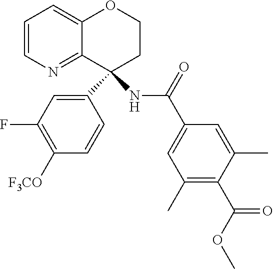 [SMILES: COC(=O)c1c(C)cc(C(=O)N[C@]2(c3ccc(OC(F)(F)F)c(F)c3)CCOc3cccnc32)cc1C] | 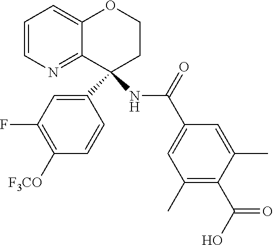 [SMILES: Cc1cc(C(=O)N[C@]2(c3ccc(OC(F)(F)F)c(F)c3)CCOc3cccnc32)cc(C)c1C(=O)O] | (S)-4-((4-(3- fluoro-4- (trifluoro- methoxy) phenyl)-3,4- dihydro-2H- pyrano[3,2-b] pyridin-4-yl) carbamoyl)-2,6- dimethylbenzoic acid | 505.0 |
| 312^{c} | 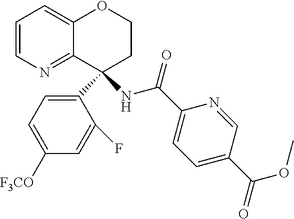 [SMILES: COC(=O)c1ccc(C(=O)N[C@]2(c3ccc(OC(F)(F)F)cc3F)CCOc3cccnc32)nc1] | 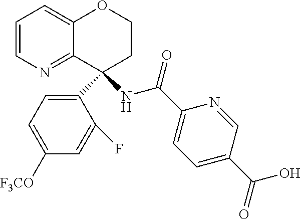 [SMILES: O=C(O)c1ccc(C(=O)N[C@]2(c3ccc(OC(F)(F)F)cc3F)CCOc3cccnc32)nc1] | (S)-6-((4-(2- fluoro-4- (trifluoro- methoxy) phenyl)-3,4- dihydro-2H- pyrano[3,2-b] pyridin-4-yl) carbamoyl) nicotinic acid | 478.0 |
| 313^{c} | 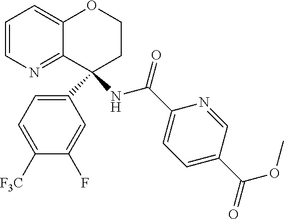 [SMILES: COC(=O)c1ccc(C(=O)N[C@]2(c3ccc(C(F)(F)F)c(F)c3)CCOc3cccnc32)nc1] | 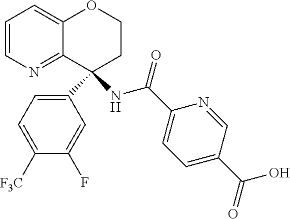 [SMILES: O=C(O)c1ccc(C(=O)N[C@]2(c3ccc(C(F)(F)F)c(F)c3)CCOc3cccnc32)nc1] | (S)-6-((4-(3- fluoro-4- (trifluoro- methyl) phenyl)-3,4- dihydro-2H- pyrano[3,2-b] pyridin-4-yl) carbamoyl) nicotinic acid | 462.0 |
| 314 | See Table 14 | | | |
| 315 | See Table 16 | | | |
| 316^{d} | 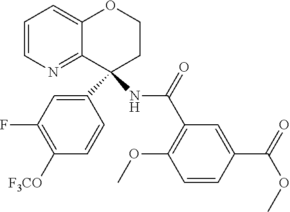 [SMILES: COC(=O)c1ccc(OC)c(C(=O)N[C@]2(c3ccc(OC(F)(F)F)c(F)c3)CCOc3cccnc32)c1] | 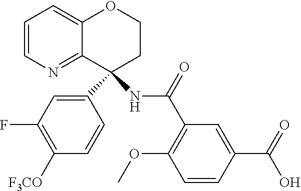 [SMILES: COc1ccc(C(=O)O)cc1C(=O)N[C@]1(c2ccc(OC(F)(F)F)c(F)c2)CCOc2cccnc21] | (S)-3-((4-(3- fluoro-4- (trifluoro- methoxy) phenyl)-3,4- dihydro-2H- pyrano[3,2-b] pyridin-4-yl) carbamoyl)-4- methoxybenzoic acid | 507.0 |
| 317^{c} | 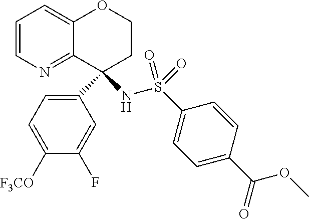 [SMILES: COC(=O)c1ccc(S(=O)(=O)N[C@]2(c3ccc(OC(F)(F)F)c(F)c3)CCOc3cccnc32)cc1] | 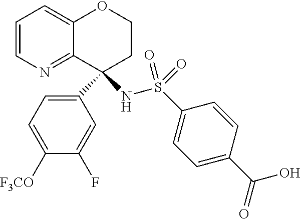 [SMILES: O=C(O)c1ccc(S(=O)(=O)N[C@]2(c3ccc(OC(F)(F)F)c(F)c3)CCOc3cccnc32)cc1] | (S)-4-(N-(4-(3- fluoro-4- (trifluoro- methoxy) phenyl)-3,4- dihydro-2H- pyrano[3,2-b] pyridin-4-yl) sulfamoyl) benzoic acid | 513.0 |
| 318^{f} | 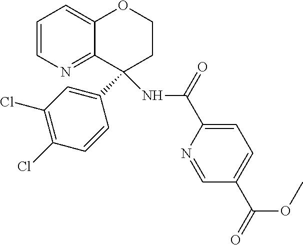 [SMILES: COC(=O)c1ccc(C(=O)N[C@]2(c3ccc(Cl)c(Cl)c3)CCOc3cccnc32)nc1] | 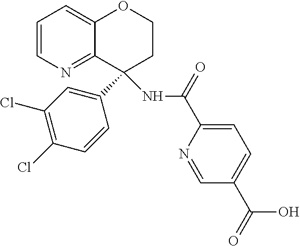 [SMILES: O=C(O)c1ccc(C(=O)N[C@]2(c3ccc(Cl)c(Cl)c3)CCOc3cccnc32)nc1] | (S)-6-((4-(3,4- dichloro- phenyl)-3,4- dihydro-2H- pyrano[3,2-b] pyridin-4-yl) carbamoyl) nicotinic acid | |
| 319^{g} | 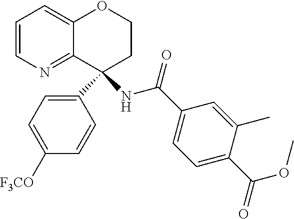 [SMILES: COC(=O)c1ccc(C(=O)N[C@]2(c3ccc(OC(F)(F)F)cc3)CCOc3cccnc32)cc1C] | 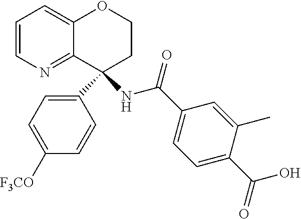 [SMILES: Cc1cc(C(=O)N[C@]2(c3ccc(OC(F)(F)F)cc3)CCOc3cccnc32)ccc1C(=O)O] | (S)-2-methyl-4- ((4-(4- (trifluoro- methoxy) phenyl)-3,4- dihydro-2H- pyrano[3,2-b] pyridin-4-yl) carbamoyl) benzoic acid | 473.0 |
| 320^{h} | 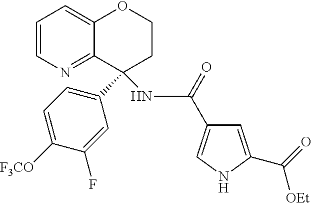 [SMILES: CCOC(=O)c1cc(C(=O)N[C@]2(c3ccc(OC(F)(F)F)c(F)c3)CCOc3cccnc32)c[nH]1] | 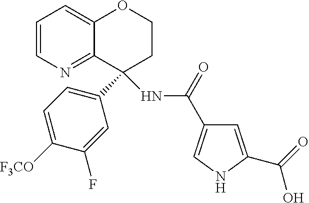 [SMILES: O=C(N[C@]1(c2ccc(OC(F)(F)F)c(F)c2)CCOc2cccnc21)c1c[nH]c(C(=O)O)c1] | (S)-4-((4-(3- fluoro-4- (trifluoro- methoxy) phenyl)-3,4- dihydro-2H- pyrano[3,2-b] pyridin-4-yl) carbamoyl)- 1H-pyrrole-2- carboxylic acid | 466.0 |
| 321ⁱ | 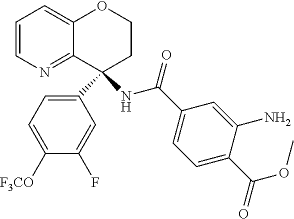 [SMILES: COC(=O)c1ccc(C(=O)N[C@]2(c3ccc(OC(F)(F)F)c(F)c3)CCOc3cccnc32)cc1N] | 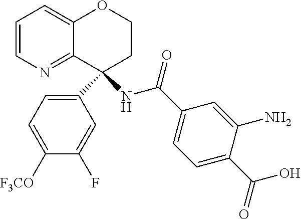 [SMILES: Nc1cc(C(=O)N[C@]2(c3ccc(OC(F)(F)F)c(F)c3)CCOc3cccnc32)ccc1C(=O)O] | (S)-2-amino-4- ((4-(3-fluoro-4- (trifluoro- methoxy) phenyl)-3,4- dihydro-2H- pyrano[3,2-b] pyridin-4-yl) carbamoyl) benzoic acid | 492.0 |
| 322^{j} | 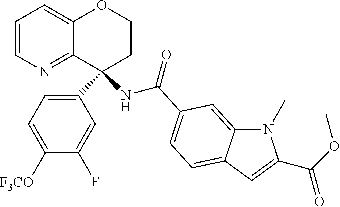 [SMILES: COC(=O)c1cc2ccc(C(=O)N[C@]3(c4ccc(OC(F)(F)F)c(F)c4)CCOc4cccnc43)cc2n1C] | 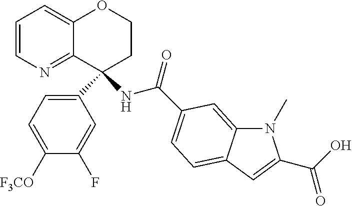 [SMILES: Cn1c(C(=O)O)cc2ccc(C(=O)N[C@]3(c4ccc(OC(F)(F)F)c(F)c4)CCOc4cccnc43)cc21] | (S)-6-((4-(3- fluoro-4- (trifluoro- methoxy) phenyl)-3,4- dihydro-2H- pyrano[3,2-b] pyridin-4-yl) carbamoyl)-1- methyl-1H- indole-2- carboxylic acid | 530.0 |
| 323^{k} | 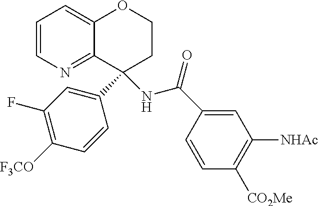 [SMILES: COC(=O)c1ccc(C(=O)N[C@]2(c3ccc(OC(F)(F)F)c(F)c3)CCOc3cccnc32)cc1NC(C)=O] | 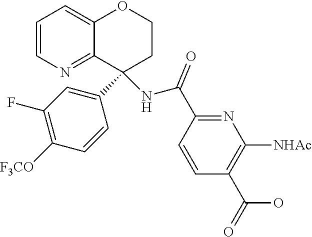 [SMILES: CC(=O)Nc1nc(C(=O)N[C@]2(c3ccc(OC(F)(F)F)c(F)c3)CCOc3cccnc32)ccc1C(=O)O] | (S)-2- acetamido-4- ((4-(3-fluoro-4- (trifluoro- methoxy) phenyl)-3,4- dihydro-2H- pyrano[3,2-b] pyridin-4-yl) carbamoyl) benzoic acid | 534.0 |
| 324ⁱ | 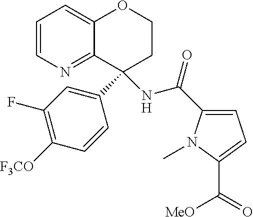 [SMILES: COC(=O)c1ccc(C(=O)N[C@]2(c3ccc(OC(F)(F)F)c(F)c3)CCOc3cccnc32)n1C] | 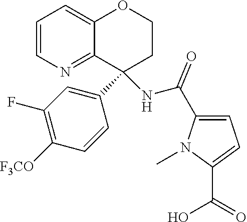 [SMILES: Cn1c(C(=O)O)ccc1C(=O)N[C@]1(c2ccc(OC(F)(F)F)c(F)c2)CCOc2cccnc21] | (S)-5-((4-(3- fluoro-4- (trifluoro- methoxy) phenyl)-3,4- dihydro-2H- pyrano[3,2-b] pyridin-4-yl) carbamoyl)- 1-methyl-1H- pyrrole-2- carboxylic acid | 480.0 |
| 325^{l} | 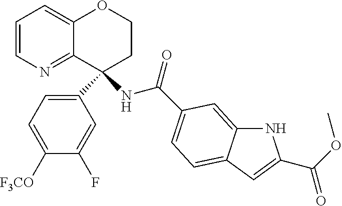 [SMILES: COC(=O)c1cc2ccc(C(=O)N[C@]3(c4ccc(OC(F)(F)F)c(F)c4)CCOc4cccnc43)cc2[nH]1] | 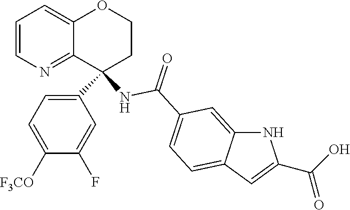 [SMILES: O=C(N[C@]1(c2ccc(OC(F)(F)F)c(F)c2)CCOc2cccnc21)c1ccc2cc(C(=O)O)[nH]c2c1] | (S)-6-((4-(3- fluoro-4- (trifluoro- methoxy) phenyl)-3,4- dihydro-2H- pyrano[3,2-b] pyridin-4-yl) carbamoyl)-1H- indole-2- carboxylic acid | 516.0 |
| 326^{m} | 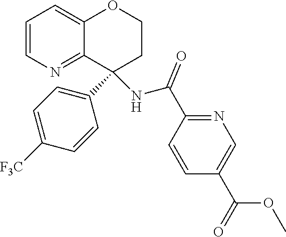 [SMILES: COC(=O)c1ccc(C(=O)N[C@]2(c3ccc(C(F)(F)F)cc3)CCOc3cccnc32)nc1] | 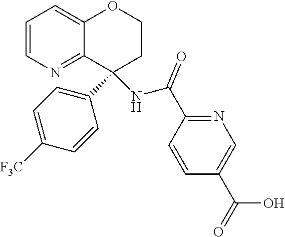 [SMILES: O=C(O)c1ccc(C(=O)N[C@]2(c3ccc(C(F)(F)F)cc3)CCOc3cccnc32)nc1] | (S)-6-((4-(4- (trifluoro- methyl) phenyl)-3,4- dihydro-2H- pyrano[3,2-b] pyridin-4-yl) carbamoyl) nicotinic acid | 444.0 |
| 327^{m} | 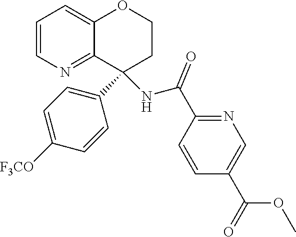 [SMILES: COC(=O)c1ccc(C(=O)N[C@]2(c3ccc(OC(F)(F)F)cc3)CCOc3cccnc32)nc1] | 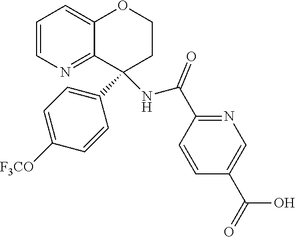 [SMILES: O=C(O)c1ccc(C(=O)N[C@]2(c3ccc(OC(F)(F)F)cc3)CCOc3cccnc32)nc1] | (S)-6-((4-(4- (trifluoro- methoxy) phenyl)-3,4- dihydro-2H- pyrano[3,2-b] pyridin-4-yl) carbamoyl) nicotinic acid | 460.0 |
| 328 | 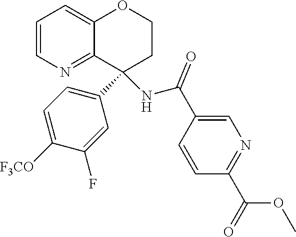 [SMILES: COC(=O)c1ccc(C(=O)N[C@]2(c3ccc(OC(F)(F)F)c(F)c3)CCOc3cccnc32)cn1] | 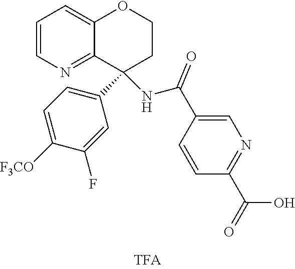 [SMILES: O=C(N[C@]1(c2ccc(OC(F)(F)F)c(F)c2)CCOc2cccnc21)c1ccc(C(=O)O)nc1] | (S)-5-((4-(3- fluoro-4- (trifluoro- methoxy) phenyl)-3,4- dihydro-2H- pyrano[3,2-b] pyridin-4-yl) carbamoyl) picolinic acid 2,2,2- trifluoroacetate | 478.0 |
| 329^{o} | 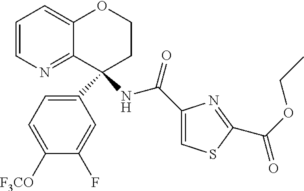 [SMILES: CCOC(=O)c1nc(C(=O)N[C@]2(c3ccc(OC(F)(F)F)c(F)c3)CCOc3cccnc32)cs1] | 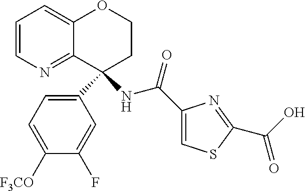 [SMILES: O=C(N[C@]1(c2ccc(OC(F)(F)F)c(F)c2)CCOc2cccnc21)c1csc(C(=O)O)n1] | (S)-4-((4-(3- fluoro-4- (trifluoro- methoxy) phenyl)-3,4- dihydro-2H- pyrano[3,2-b] pyridin-4-yl) carbamoyl) thiazole-2- carboxylic acid | 484.0 |
| 330^{p} | 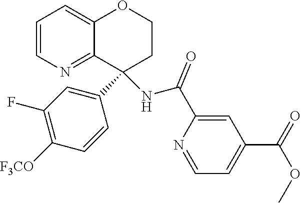 [SMILES: COC(=O)c1ccnc(C(=O)N[C@]2(c3ccc(OC(F)(F)F)c(F)c3)CCOc3cccnc32)c1] | 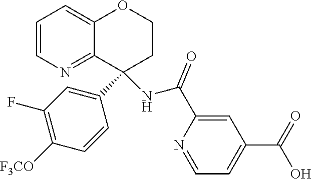 [SMILES: O=C(O)c1ccnc(C(=O)N[C@]2(c3ccc(OC(F)(F)F)c(F)c3)CCOc3cccnc32)c1] | (S)-2-((4-(3- fluoro-4- (trifluoro- methoxy) phenyl)-3,4- dihydro-2H- pyrano[3,2-b] pyridin-4-yl) carbamoyl) isonicotinic acid | 478.0 |

^{a}5N NaOH in H₂O (2 eq) at room temperature for 3.5 h.
^{b}2M LiOH in H₂O (4 eq) at 65° C. for 89 h.
^{c}1M LiOH in H₂O (2 eq) at room temperature for 2 h.
^{d}LiOH (5 eq) in MeOH/H₂O (4:1) at room temperature for 24 h, then 60° C. for 2 h
^{e}LiOH (2 eq) in THF/H₂O at room temperature overnight.
^{f}LiOH (2 eq) in THF/H₂O (3:1) at 0° C. for 2 h.
^{g}5N NaOH (in H₂O, 5 eq) in MeOH at 55° C. 3 h.
^{h}5N NaOH (in H₂O, 5 eq) in MeOH at room temperature for 2 d.
ⁱ5N NaOH (in H₂O, 5 eq) in MeOH at room temperature overnight.
^{j}1M LiOH (in H₂O, 10 eq) at room temperature for 2 h.
^{k}5N NaOH (in H₂O, 5 eq) in MeOH at room temperature for 16 h.
^{l}1M NaOH (in H₂O, 3 eq) at room temperature for 26 h.
^{m}1M LiOH (in H₂O, 5 eq) at room temperature for 2 h.
ⁿ1M LiOH (in H₂O, 10 eq) at room temperature for 2 h.
^{o}1M LiOH (in H₂O, 1 eq) at room temperature for 24 h
^{p}1M LiOH (in H₂O, 3.5 eq) at room temperature for 2 h.

### General Procedure for Carbonylation

A mixture of bromide intermediate (1 eq), K₂CO₃ or TEA (4 eq), (9,9-dimethyl-9H-xanthene-4,5-diyl)bis(diphenylphosphine) (0.2 eq) and bis(tri-t-butylphosphine)palladium (0) or Pd(OAc)₂ (0.4 eq) was capped, evacuated under vacuum and backfilled with argon (2×). DMF (2 mL) and water (10 eq) were added. CO (in a balloon) was bubbled through the reaction mixture for 30 sec, after which the reaction was heated to 80° C. under a CO balloon. After 16 h, the reaction was cooled to room temperature, acidified with 5N HCl, diluted with EtOAc (50 mL) and washed with water (50 mL) and brine (50 mL), dried over MgSO₄, concentrated in vacuo and purified by silica gel flash chromatography or preparative HPLC to give the title compound.

### Example 332

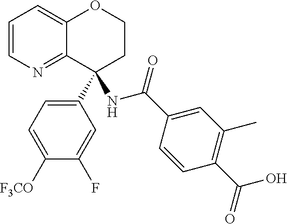 [SMILES: Cc1cc(C(=O)N[C@]2(c3ccc(OC(F)(F)F)c(F)c3)CCOc3cccnc32)ccc1C(=O)O]

### (S)-4-((4-(3-fluoro-4-(trifluoromethoxy)phenyl)-3,4-dihydro-2H-pyrano[3,2-b]pyridin-4-yl)carbamoyl)-2-methylbenzoic acid

A microwave vial containing (S)-4-bromo-N-(4-(3-fluoro-4-(trifluoromethoxy)phenyl)-3,4-dihydro-2H-pyrano[3,2-b]pyridin-4-yl)-3-methylbenzamide (93 mg, 0.177 mmol), bis(tri-t-butylphosphine)palladium (0) (36.2 mg, 0.071 mmol), (9,9-dimethyl-9H-xanthene-4,5-diyl)bis(diphenylphosphine) (20.49 mg, 0.035 mmol), and potassium carbonate (98 mg, 0.708 mmol) was flushed with N₂ (3×), and then DMF (1 mL) and water (0.032 mL, 1.770 mmol) were added. CO gas was bubbled through the suspension for 5 minutes and then the reaction was heated in a 80° C. oil bath and stirred under 1 atm of CO (balloon). The reaction was stirred for 3 h, and then it was poured into 1N aqueous HCl (2 mL) and diluted with water (10 mL). The resulting mixture was extracted with EtOAc (3×10 mL) (brine (5 mL) was added to the first extraction to dissolve an emulsion). The combined organic layers were dried (MgSO₄) and concentrated. The residue was dissolved in DMF (2 mL), and the product was purified by reverse phase HPLC (Gemini Axia C18 50×150 mm column, gradient elution 10-100% MeCN/0.1% TFA in H₂O). The product containing fractions were combined, and the solvent was removed by lyophilization to give the title compound as an off white solid. MS (ESI pos. ion) m/z: 490.9 (MH+). ¹H NMR (400 MHz, DMSO-d₆) δ: 13.02 (br. s., 1H), 8.96 (s, 1H), 8.12 (dd, J=4.2, 1.7 Hz, 1H), 7.83 (d, J=8.0 Hz, 1H), 7.72 (s, 1H), 7.69 (d, J=8.2 Hz, 1H), 7.44-7.57 (m, 2H), 7.19-7.32 (m, 3H), 4.34 (ddd, J=11.2, 7.9, 2.6 Hz, 1H), 4.13 (ddd, J=11.1, 7.8, 2.6 Hz, 1H), 3.27 (td, J=7.0, 2.7 Hz, 1H), 2.86 (ddd, J=14.1, 7.8, 2.5 Hz, 1H), 2.54 (s, 3H).

TABLE 16
Examples 315 and 331-337 prepared via carbonylation analogous
to general procedures described above.

| | | Product | Product | MS |
|---|---|---|---|---|
| Ex # | Bromide precursor | Structure | Name | M + H |
| 315 | 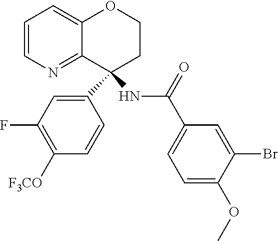 [SMILES: COc1ccc(C(=O)N[C@]2(c3ccc(OC(F)(F)F)c(F)c3)CCOc3cccnc32)cc1Br] | 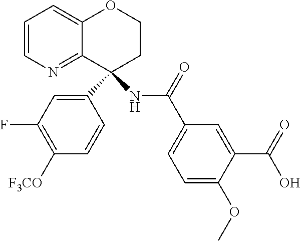 [SMILES: COc1ccc(C(=O)N[C@]2(c3ccc(OC(F)(F)F)c(F)c3)CCOc3cccnc32)cc1C(=O)O] | (S)-5-((4-(3- fluoro-4- (trifluoromethoxy) phenyl)-3,4- dihydro-2H- pyrano[3,2-b] pyridin-4-yl) carbamoyl)-2- methoxybenzoic acid | 506.9 |
| 331 | 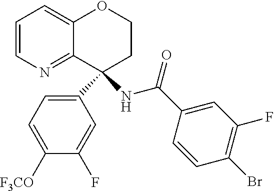 [SMILES: O=C(N[C@]1(c2ccc(OC(F)(F)F)c(F)c2)CCOc2cccnc21)c1ccc(Br)c(F)c1] | 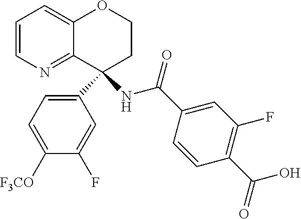 [SMILES: O=C(N[C@]1(c2ccc(OC(F)(F)F)c(F)c2)CCOc2cccnc21)c1ccc(C(=O)O)c(F)c1] | (S)-2-fluoro- 4-((4-(3-fluoro-4- (trifluoromethoxy) phenyl)-3,4- dihydro-2H- pyrano[3,2-b] pyridin-4-yl) carbamoyl) benzoic acid | 495.0 |
| 332 | 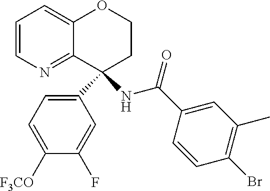 [SMILES: Cc1cc(C(=O)N[C@]2(c3ccc(OC(F)(F)F)c(F)c3)CCOc3cccnc32)ccc1Br] | 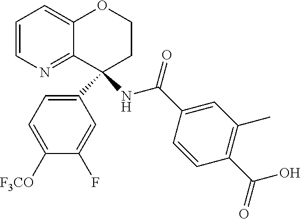 [SMILES: Cc1cc(C(=O)N[C@]2(c3ccc(OC(F)(F)F)c(F)c3)CCOc3cccnc32)ccc1C(=O)O] | (S)-4-((4-(3- fluoro-4- (trifluoromethoxy) phenyl)-3,4- dihydro-2H- pyrano[3,2-b] pyridin-4-yl) carbamoyl)-2- methylbenzoic acid | 490.9 |
| 333 | 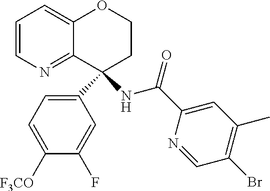 [SMILES: Cc1cc(C(=O)N[C@]2(c3ccc(OC(F)(F)F)c(F)c3)CCOc3cccnc32)ncc1Br] | 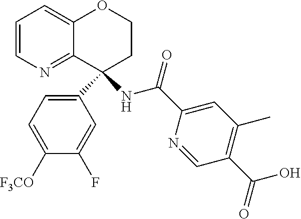 [SMILES: Cc1cc(C(=O)N[C@]2(c3ccc(OC(F)(F)F)c(F)c3)CCOc3cccnc32)ncc1C(=O)O] | (S)-6-((4-(3- fluoro-4- (trifluoromethoxy) phenyl)-3,4- dihydro-2H- pyrano[3,2-b] pyridin-4-yl) carbamoyl)-4- methylnicotinic acid | 491.9 |
| Inter- mediate 49 | 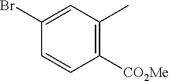 | 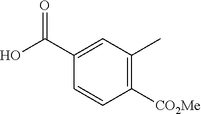 | 4-(methoxy- carbonyl)-3- methylbenzoic acid | 195 |
| 334 | 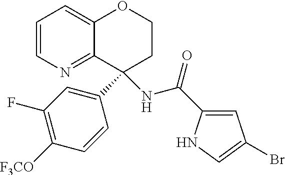 [SMILES: O=C(N[C@]1(c2ccc(OC(F)(F)F)c(F)c2)CCOc2cccnc21)c1cc(Br)c[nH]1] | 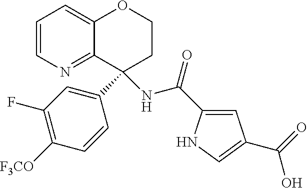 [SMILES: O=C(O)c1c[nH]c(C(=O)N[C@]2(c3ccc(OC(F)(F)F)c(F)c3)CCOc3cccnc32)c1] | (S)-5-((4-(3- fluoro-4- (trifluoromethoxy) phenyl)-3,4- dihydro-2H- pyrano[3,2-b] pyridin-4-yl) carbamoyl)- 1H-pyrrole-3- carboxylic acid | 466.0 |
| 335 | 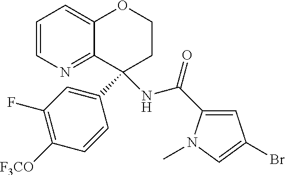 [SMILES: Cn1cc(Br)cc1C(=O)N[C@]1(c2ccc(OC(F)(F)F)c(F)c2)CCOc2cccnc21] | 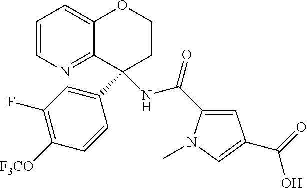 [SMILES: Cn1cc(C(=O)O)cc1C(=O)N[C@]1(c2ccc(OC(F)(F)F)c(F)c2)CCOc2cccnc21] | (S)-5-((4-(3- fluoro-4- (trifluoromethoxy) phenyl)-3,4- dihydro-2H- pyrano[3,2-b] pyridin-4-yl) carbamoyl)-1- methyl- 1H-pyrrole- 3-carboxylic acid | 480.0 |
| 336 | 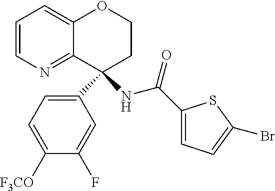 [SMILES: O=C(N[C@]1(c2ccc(OC(F)(F)F)c(F)c2)CCOc2cccnc21)c1ccc(Br)s1] | 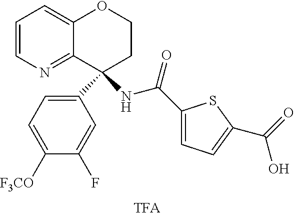 [SMILES: O=C(O)c1ccc(C(=O)N[C@]2(c3ccc(OC(F)(F)F)c(F)c3)CCOc3cccnc32)s1] | (S)-5-((4-(3- fluoro-4- (trifluoromethoxy) phenyl)-3,4- dihydro-2H- pyrano[3,2-b] pyridin-4-yl) carbamoyl) thiophene-2- carboxylic acid 2,2,2- trifluoroacetate | 482.9 |
| 337 | 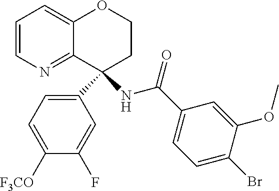 [SMILES: COc1cc(C(=O)N[C@]2(c3ccc(OC(F)(F)F)c(F)c3)CCOc3cccnc32)ccc1Br] | 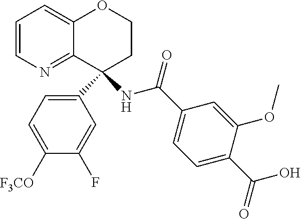 [SMILES: COc1cc(C(=O)N[C@]2(c3ccc(OC(F)(F)F)c(F)c3)CCOc3cccnc32)ccc1C(=O)O] | (S)-4-((4-(3- fluoro-4- (trifluoromethoxy) phenyl)-3,4- dihydro-2H- pyrano[3,2-b] pyridin-4-yl) carbamoyl)-2- methoxybenzoic acid | 506.9 |
| Inter- medite 50 | 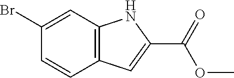 | 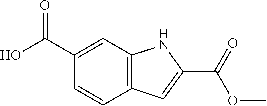 [SMILES: COC(=O)c1cc2ccc(C(=O)O)cc2[nH]1] | 2- (methoxycarbonyl)- 1H-indole-6- carboxylic acid | 220.1 |
| Inter- mediate 51 | 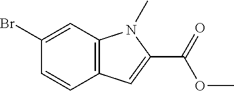 | 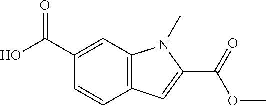 [SMILES: COC(=O)c1cc2ccc(C(=O)O)cc2n1C] | 2- (methoxycarbonyl)- 1-methyl-1H- indole-6- carboxylic acid | 234.1 |

TABLE 17
¹H NMR Data of Examples 1-337.

| | Freq., | |
|---|---|---|
| Ex. # | Solvent | ¹HNMR Data (δ ppm) |
| 1 | 300 MHz | 8.19 (dd, J = 4.0, 2.0 Hz, 1H), 7.74-7.86 (m, 2H), 7.65 (d, J = 8.6 Hz, |
| | d₄-MeOH | 2H), 7.58 (d, J = 8.5 Hz, 2H), 7.52 (d, J = 7.3 Hz, 1H), 7.40-7.49 (m, |
| | | 2H), 7.28-7.38 (m, 2H), 4.33-4.47 (m, 1H), 4.07 (td, J = 11.1, 2.5 Hz, |
| | | 1H), 3.33-3.40 (m, 1H), 3.01-3.16 (m, 1H) |
| 2 | 300 MHz | 8.18 (dd, J = 4.1, 1.9 Hz, 1H), 7.77-7.92 (m, 2H), 7.61-7.71 (m, J = 8.5 Hz, |
| | d₄-MeOH | 2H), 7.51-7.61 (m, J = 8.6 Hz, 2H), 7.26-7.39 (m, 2H), |
| | | 7.10-7.25 (m, 2H), 4.31-4.47 (m, 1H), 3.99-4.14 (m, 1H), 3.33-3.42 (m, |
| | | 1H), 2.94-3.10 (m, 1H) |
| 3 | 400 MHz, | 8.9 (s, 1H), 8.3 (m, 1H), 7.81 (d, J = 7.2 Hz, 1H), 7.6 (s, 1H), |
| | CDCl₃ | 7.5-7.6 (m, 2H), 7.45 (t, J = 7.2 Hz, 3H), 7.2-7.4 (m, 3H), 4.3 (m, 1H), |
| | | 4.01-4.15 (m, 1H), 3.2 (m, 1H), 2.91 (m, 1H) |
| 4 | 300 MHz, | 8.28 (t, J = 2.9 Hz, 1H), 8.05 (s, 1H), 7.40-7.70 (m, 6H), 7.29 (d, J = 3.1 Hz, |
| | CDCl₃ | 2H), 6.68 (d, J = 8.6 Hz, 2H), 4.38 (dt, J = 11.6, 3.6 Hz, 1H), |
| | | 3.99 (td, J = 12.1, 2.0 Hz, 1H), 3.36-3.47 (m, 1H), 2.97-3.27 (m, 1H) |
| 5 | 300 MHz, | 8.37 (s, 1H), 8.30 (dd, J = 3.6, 2.3 Hz, 1H), 7.70-7.89 (m, 2H), |
| | CDCl₃ | 7.48-7.63 (m, 4H), 7.27 (d, J = 1.9 Hz, 1H), 6.84-6.99 (m, 2H), 4.40 (dt, J = 11.7, |
| | | 3.8 Hz, 1H), 4.04 (td, J = 12.1, 2.2 Hz, 1H), 3.84 (s, 3H), 3.73-3.82 (m, |
| | | 1H), 2.89 (ddd, J = 14.7, 12.5, 4.2 Hz, 1H) |
| 6 | 400 MHz, | 8.92 (s, 1H), 8.11-8.12 (m, 1H), 7.88-7.91 (m, 2H), 7.46-7.55 (m, 2H), |
| | DMSO-d₆ | 7.20-7.31 (m, 5H), 4.30-4.33 (m, 1H), 4.11-4.13 (m, 1H), 3.25-3.34 (m, |
| | | 1H), 2.84-2.86 (m, 1H). |
| 7 | 400 MHz, | 8.89 (s, 1H), 8.12-8.13 (m, 1H), 7.81 (d, J = 7.6 Hz, 1H), 7.42-7.54 (m, |
| | DMSO-d₆ | 5H), 7.22-7.31 (m, 3H), 4.31-4.36 (m, 1H), 4.11-4.15 (m, 1H), |
| | | 3.23-3.27 (m, 1H), 2.88-2.91 (m, 1H). |
| 8 | 400 MHz, | δ 8.5 (s, 1H), 8.3 (dd, J = 2.0, 4.0 Hz, 1H), 7.88-7.91 (q, d, J = 5.2, 8.8 Hz, |
| | CDCl₃ | 2H), 7.4 (d, J = 8.8 Hz, 2H), 7.1 (m, 4H), 4.4 (m, 1H), 4.0 (m, 1H), |
| | | 3.8 (m, 1H), 2.84-2.86 (m, 1H). |
| 9 | 400 MHz, | 8.5 (s, 1H), 8.3 (m, 1H), 7.81 (d, J = 7.2 Hz, 1H), 7.42-7.54 (m, 5H), |
| | CDCl₃ | 7.1 (d, J = 7.2 Hz, 3H), 4.4-4.42 (m, 1H), 4.01-4.15 (m, 1H), 3.7-3.8 (m, |
| | | 1H), 2.88-2.91 (m, 1H). |
| 10 | 400 MHz, | 8.9 (s, 1H), 8.1 (dd, J = 2.0, 4.0 Hz, 1H), 7.88-7.91 (q, J = 5.2, 8.8 Hz, |
| | DMSO-d₆ | 2H), 7.6 (m, 2H), 7.3 (m, 5H), 4.3 (m, 1H), 4.1 (m, 1H), 3.2 (m, 1H), |
| | | 2.86 (m, 1H). |
| 11 | 400 MHz, | 8.73 (s, 1H), 8.14 (dd, J = 0.8, 1.6 Hz, 1H), 7.89 (dd, J = 0.8, 1.6 Hz |
| | DMSO-d₆ | 2H), 7.32-7.23 (m, 8H), 4.3 (m, 1H), 3.94 (m, 1H), 3.31 (m, 1H), 2.8 (m, |
| | | 1H). |
| 12 | 400 MHz, | 8.67 (s, 1H), 8.16 (dd, J = 1.6, 4 Hz, 1H), 7.80 (dd, J = 5.2, 7.2 Hz, 2H), |
| | DMSO-d₆ | 7.52 (dd, J = 6.4, 7.6 Hz, 1H), 7.45 (t, J = 8, 15.2 Hz, 2H), 7.34-7.25 (m, |
| | | 7H), 4.32 (m, 1H), 3.96 (m, 1H), 3.26 (m, 1H), 2.89 (m, 1H). |
| 13 | 400 MHz, | 9.14 (s, 1H), 8.84 (d, J = 2 Hz, 1H), 8.09-8.04 (m, 2H), 7.85 (t, J = 6, |
| | DMSO-d₆ | 7.6 Hz, 3H), 7.55 (t, J = 7.2, 2.8 Hz, 1H), 7.46 (t, J = 8, 15.2 Hz, 2H), |
| | | 7.33-7.24 (m, 2H), 4.40-4.35 (m, 1H), 4.33-4.29 (m, 1H), |
| | | 3.25-3.20 (m, 1H), 3.03-2.97 (m, 1H). |
| 14 | 400 MHz, | 8.47 (s, 1H), 8.29 (dd, J = 3.8, 2.1 Hz, 1H), 7.73-7.86 (m, 2H), |
| | DMSO-d₆ | 7.33-7.53 (m, 5H), 7.21-7.30 (m, 4H), 4.39 (dt, J = 11.5, 3.7 Hz, 1H), |
| | | 3.97-4.09 (m, 1H), 3.75 (dt, J = 14.5, 2.6 Hz, 1H), 2.85 (ddd, J = 14.5, 12.9, |
| | | 4.1 Hz, 1H) |
| 15 | 400 MHz, | 8.48 (s, 1H), 8.30 (dd, J = 4.1, 1.8 Hz, 1H), 7.74-7.87 (m, 2H), |
| | CDCl₃ | 7.46-7.52 (m, 1H), 7.42 (t, J = 7.3 Hz, 2H), 7.26-7.34 (m, 3H), 7.18-7.25 (m, 1H), |
| | | 4.41 (dt, J = 11.7, 3.6 Hz, 1H), 3.98-4.11 (m, 1H), 3.79 (dt, J = 14.8, 2.7 Hz, |
| | | 1H), 2.81 (ddd, J = 14.7, 12.8, 4.2 Hz, 1H) |
| 16 | 400 MHz, | 8.49 (s, 1H), 8.29 (dd, J = 3.7, 2.2 Hz, 1H), 7.72-7.89 (m, 2H), |
| | CDCl₃ | 7.32-7.54 (m, 5H), 7.17-7.32 (m, 3H), 6.89-7.04 (m, 2H), 4.39 (dt, J = 11.2, |
| | | 3.8 Hz, 1H), 3.95-4.12 (m, 1H), 3.74 (dt, J = 14.6, 2.5 Hz, 1H), |
| | | 2.86 (ddd, J = 14.5, 13.0, 4.2 Hz, 1H) |
| 17 | 400 MHz, | 8.46 (s, 1H), 8.30 (dd, J = 3.7, 2.2 Hz, 1H), 7.72-7.88 (m, 2H), |
| | CDCl₃ | 7.36-7.54 (m, 4H), 7.32 (t, J = 8.0 Hz, 1H), 7.27-7.30 (m, 2H), 7.11 (d, J = 8.2 Hz, |
| | | 1H), 4.40 (dt, J = 11.6, 3.7 Hz, 1H), 3.95-4.10 (m, 1H), |
| | | 3.76 (dt, J = 14.7, 2.5 Hz, 1H), 2.88 (ddd, J = 14.5, 13.0, 4.2 Hz, 1H) |
| 18 | 400 MHz, | 8.40 (s, 1H), 8.27 (t, J = 2.9 Hz, 1H), 7.72-7.87 (m, 2H), 7.43-7.50 (m, |
| | CDCl₃ | 1H), 7.36-7.43 (m, 2H), 7.28 (d, J = 8.4 Hz, 2H), 7.23 (d, J = 2.9 Hz, |
| | | 2H), 7.09 (d, J = 8.0 Hz, 2H), 4.36 (dt, J = 11.4, 3.6 Hz, 1H), |
| | | 3.98-4.13 (m, 1H), 3.65 (dt, J = 14.4, 2.5 Hz, 1H), 2.93 (ddd, J = 14.3, 13.0, 4.2 Hz, |
| | | 1H), 2.29 (s, 3H) |
| 19 | 400 MHz, | 8.46 (s, 1H), 8.31 (dd, J = 4.1, 1.8 Hz, 1H), 7.76-7.83 (m, 2H), |
| | CDCl₃ | 7.47-7.56 (m, 2H), 7.43 (t, J = 7.3 Hz, 2H), 7.33-7.40 (m, 2H), 7.27-7.33 (m, 2H), |
| | | 4.43 (dt, J = 11.8, 3.9 Hz, 1H), 4.06 (td, J = 12.2, 2.2 Hz, 1H), |
| | | 3.76-3.89 (m, 1H), 2.85 (ddd, J = 14.7, 12.5, 4.1 Hz, 1H) |
| 20 | 400 MHz, | 8.79 (s, 1H), 8.10 (dd, J = 1.57, 4.30 Hz, 1H), 7.78 (d, J = 1.37 Hz, 1H), |
| | DMSO-d₆ | 7.60-7.71 (m, 3H), 7.57 (d, J = 8.41 Hz, 2H), 7.19-7.35 (m, 2H), |
| | | 7.12 (d, J = 8.22 Hz, 1H), 4.34 (ddd, J = 2.84, 8.02, 11.25 Hz, 1H), 4.13 (ddd, |
| | | J = 2.84, 7.73, 11.15 Hz, 1H), 3.20-3.27 (m, 1H), 2.86 (ddd, J = 2.74, |
| | | 7.87, 14.23 Hz, 1H). |
| 21 | 400 MHz, | 8.81 (s, 1H), 8.12 (dd, J = 1.56, 4.11 Hz, 1H), 7.78 (s, 1H), 7.68 (dd, J = 1.17, |
| | DMSO-d₆ | 8.22 Hz, 1H), 7.41-7.60 (m, 2H), 7.17-7.36 (m, 3H), 7.11 (d, J = 8.22 Hz, |
| | | 1H), 4.28-4.38 (m, 1H), 4.07-4.17 (m, 1H), 3.22-3.27 (m, 1H), |
| | | 2.71-2.91 (m, 1H). |
| 22 | 400 MHz, | 11.95 (br s, 1H), 8.83 (s, 1H), 8.11 (d, J = 2.4 Hz, 1H), 7.79 (s, 1H), |
| | DMSO-d₆ | 7.68 (d, J = 8.4 Hz, 1H), 7.4 (d, J = 8.8 Hz, 2H), 7.2-7.4 (m, 4H), 7.11 (d, J = 8.4 Hz, |
| | | 1H), 4.30-4.34 (m, 1H), 4.10-4.14 (m, 1H), 2.81-2.86 (m, 1H). |
| 23 | 400 MHz, | 11.95 (br s, 1H), 8.82 (s, 1H), 8.11 (dd, J = 1.6, 4.4 Hz, 1H), |
| | DMSO-d₆ | 7.53-7.60 (dd, J = 1.2, 8.4 Hz, 1H), 7.5 (s, 1H), 7.4 (d, J = 8.8 Hz, 1H), |
| | | 7.2-7.35 (m, 5H), 4.31-4.38 (m, 1H), 4.09-4.15 (m, 1H), 2.82-2.89 (m, 1H). |
| 24 | 400 MHz, | 11.95 (br s, 1H), 8.82 (s, 1H), 8.11 (s, 1H), 7.79 (s, 1H), 7.68 (d, J = 8.0 Hz, |
| | DMSO-d₆ | 1H), 7.5-7.6 (m, 2H), 7.3 (m, 3H), 7.11 (d, J = 8.4 Hz, 1H), |
| | | 4.30-4.34 (m, 1H), 4.10-4.14 (m, 1H), 3.2 (m, 1H), 2.81-2.86 (m, 1H). |
| 25 | 400 MHz, | 11.80 (br s, 1H), 8.9 (s, 1H), 8.11 (d, J = 4.4 Hz, 1H), 7.53-7.65 (m, 4H), |
| | DMSO-d₆ | 7.2-7.4 (m, 4H), 4.31-4.38 (t, J = 8.8 Hz, 1H), 4.1-4.8 (t, J = 8.4 Hz, 1H), |
| | | 3.2 (m, 1H), 2.82-2.89 (m, 1H). |
| 26 | 400 MHz, | 11.95 (br s, 1H), 8.57 (s, 1H), 8.13 (s, 1H), 7.72 (s, 1H), 7.66 (d, J = 8.0 Hz, |
| | DMSO-d₆ | 1H), 7.33-7.32 (m, 2H), 7.29-7.27 (m, 5H), 7.15 (d, J = 8.0 Hz, 1H), |
| | | 4.33-4.30 (m, 1H), 3.97-3.92 (m, 1H), 3.28 (m, 1H), 2.80-2.77 (m, 1H). |
| 27 | 400 MHz, | 11.99 (s, 1H), 9.16 (s, 1H), 8.83 (s, 1H), 8.09 (dd, J = 2.8, 13.6 Hz 2H), |
| | DMSO-d₆ | 7.85 (d, J = 8.4 Hz, 1H), 7.64 (d, J = 8.4 Hz, 1H), 7.55 (s, 1H) |
| | | 7.34-7.25 (m, 3H), 4.36-4.30 (m, 2H), 3.24-3.20 (m, 1H), 3.00-2.98 (m, 1H). |
| 28 | 300 MHz, | 7.74-7.85 (m, 2H), 7.60 (d, J = 8.3 Hz, 2H), 7.50-7.58 (m, 1H), |
| | CDCl₃ | 7.41-7.50 (m, 4H), 7.18-7.24 (m, 1H), 6.93 (dd, J = 8.3, 0.9 Hz, 1H), |
| | | 6.71-6.88 (m, 2H), 6.50 (s, 1H), 4.23-4.47 (m, 2H), 3.54-3.70 (m, 1H), |
| | | 2.58 (ddd, J = 14.5, 10.5, 4.2 Hz, 1H) |
| 29 | 300 MHz, | 8.46-8.63 (m, 2H), 7.74-7.88 (m, 2H), 7.33-7.66 (m, 8H), 7.24-7.28 (m, |
| | CDCl₃ | 1H), 3.55 (dt, J = 14.0, 4.2 Hz, 1H), 2.89 (dd, J = 9.6, 6.8 Hz, 1H), |
| | | 2.54-2.82 (m, 2H), 1.86-2.01 (m, 1H), 1.65-1.83 (m, 1H) |
| 30 | 400 MHz | 9.18 (br. s., 1H), 8.51 (d, J = 8.0 Hz, 1H), 8.29 (br. s., 1H), 8.18 (d, J = 7.6 Hz, |
| | d₄-MeOH | 1H), 7.36-7.56 (m, 4H), 7.25 (d, J = 8.4 Hz, 1H), 4.46 (d, J = 11.1 Hz, |
| | | 1H), 4.13 (t, J = 11.1 Hz, 1H), 3.97 (br. s., 3H), 3.18-3.28 (m, |
| | | 1H), 3.13 (d, J = 10.4 Hz, 1H) |
| 31 | 300 MHz | 8.34-8.44 (m, 1H), 8.04 (d, J = 2.2 Hz, 1H), 7.92 (dd, J = 9.6, 2.7 Hz, |
| | d₄-MeOH | 1H), 7.57-7.74 (m, 3H), 7.25-7.40 (m, 3H), 6.50 (d, J = 9.5 Hz, 1H), |
| | | 3.16 (td, J = 12.9, 3.4 Hz, 1H), 2.94-3.09 (m, 1H), 2.80-2.93 (m, 1H), |
| | | 2.54-2.66 (m, 1H), 1.81-1.95 (m, 1H), 1.50-1.70 (m, 1H) |
| 32 | 300 MHz, | 9.42 (s, 1H), 8.70 (d, J = 2.3 Hz, 1H), 8.33 (dd, J = 5.2, 1.2 Hz, 1H), |
| | CDCl₃ | 8.09 (dd, J = 8.8, 2.5 Hz, 1H), 7.76 (dd, J = 8.6, 1.2 Hz, 1H), |
| | | 7.56-7.71 (m, 3H), 7.34 (d, J = 8.3 Hz, 2H), 6.75 (d, J = 8.8 Hz, 1H), 4.43-4.56 (m, |
| | | 1H), 3.91-4.06 (m, 4H), 3.61-3.80 (m, 1H), 2.60 (d, J = 14.2 Hz, 1H) |
| 33 | 300 MHz, | 8.41 (s, 1H), 8.31 (dd, J = 3.7, 2.1 Hz, 1H), 7.74 (s, 1H), 7.66 (s, 1H), |
| | CDCl₃ | 7.50-7.64 (m, 5H), 7.27-7.30 (m, 2H), 6.75 (d, J = 8.2 Hz, 1H), 4.41 (dt, |
| | | J = 11.7, 3.7 Hz, 1H), 4.03 (td, J = 12.2, 2.2 Hz, 1H), 3.79 (dt, J = 14.8, |
| | | 2.6 Hz, 1H), 2.94-3.08 (m, 2H), 2.87 (ddd, J = 14.6, 12.7, 4.2 Hz, 1H), |
| | | 2.54-2.73 (m, 2H) |
| 34 | 300 MHz | 9.42 (s, 1H), 8.34 (d, J = 4.1 Hz, 1H), 8.16 (s, 2H), 7.77-7.87 (m, 1H), |
| | d₄-MeOH | 7.51-7.77 (m, 5H), 7.40 (d, J = 8.3 Hz, 2H), 4.51 (d, J = 12.0 Hz, 1H), |
| | | 4.01 (t, J = 11.9 Hz, 1H), 3.68 (d, J = 4.5 Hz, 1H), 2.78 (d, J = 14.2 Hz, |
| | | 1H) |
| 35 | 300 MHz, | 9.27 (d, J = 1.3 Hz, 1H), 9.02 (d, J = 5.1 Hz, 1H), 8.26 (dd, J = 4.0, 2.0 Hz, |
| | CDCl₃ | 1H), 8.07 (dd, J = 5.0, 1.4 Hz, 1H), 7.63-7.74 (m, J = 8.5 Hz, 2H), |
| | | 7.52-7.63 (m, J = 8.5 Hz, 2H), 7.36-7.48 (m, 2H), 4.43 (d, J = 11.8 Hz, |
| | | 1H), 4.00-4.15 (m, 1H), 3.24 (dd, J = 4.3, 2.9 Hz, 1H), 3.17 (dd, J = 10.8, |
| | | 3.9 Hz, 1H) |
| 36 | 300 MHz | 8.31 (d, J = 5.0 Hz, 1H), 7.94-8.01 (m, 1H), 7.75-7.81 (m, 1H), 7.71 (d, J = 8.5 Hz, |
| | d₄-MeOH | 2H), 7.60-7.68 (m, 2H), 7.51-7.59 (m, 3H), 7.42 (d, J = 3.1 Hz, |
| | | 1H), 6.52 (d, J = 3.1 Hz, 1H), 4.49-4.63 (m, 1H), 4.26 (ddd, J = 11.7, |
| | | 8.6, 2.9 Hz, 1H), 3.17 (ddd, J = 14.5, 8.4, 3.4 Hz, 1H), 2.90 (ddd, J = 14.7, |
| | | 7.0, 2.9 Hz, 1H) |
| 37 | 300 MHz | 9.49-9.56 (m, 1H), 9.37 (dd, J = 5.4, 1.2 Hz, 1H), 8.24 (dd, J = 4.8, 1.5 Hz, |
| | d₄-MeOH | 1H), 8.07 (dd, J = 5.3, 2.3 Hz, 1H), 7.72 (d, J = 8.3 Hz, 2H), |
| | | 7.58-7.67 (m, 1H), 7.54 (d, J = 8.2 Hz, 3H), 4.44-4.56 (m, 1H), 4.11-4.24 (m, |
| | | 1H), 3.26 (m, 1H), 2.75-2.88 (m, 1H) |
| 38 | 300 MHz | 8.30 (dd, J = 4.8, 1.2 Hz, 1H), 8.16 (s, 1H), 8.07 (s, 1H), 7.84 (d, J = 8.6 Hz, |
| | d₄-MeOH | 1H), 7.69-7.81 (m, 3H), 7.52-7.69 (m, 4H), 4.51-4.66 (m, 1H), |
| | | 4.27 (ddd, J = 11.6, 8.4, 2.8 Hz, 1H), 4.13 (s, 3H), 3.19-3.28 (m, 1H), |
| | | 2.86 (ddd, J = 14.5, 7.2, 2.9 Hz, 1H) |
| 39 | 300 MHz | 9.16 (s, 1H), 8.58 (s, 1H), 8.15-8.33 (m, 1H), 7.72 (t, J = 7.9 Hz, 3H), |
| | d₄-MeOH | 7.47-7.66 (m, 3H), 4.44-4.60 (m, 1H), 4.22 (ddd, J = 11.6, 8.5, 2.9 Hz, |
| | | 1H), 3.23 (ddd, J = 14.5, 8.4, 3.1 Hz, 1H), 2.75-2.91 (m, 1H) |
| 40 | 300 MHz | 8.27 (dd, J = 4.8, 1.3 Hz, 1H), 8.16 (d, J = 2.6 Hz, 1H), 7.91 (d, J = 8.6 Hz, |
| | d₄-MeOH | 1H), 7.61-7.78 (m, 3H), 7.49-7.61 (m, 3H), 7.26 (dd, J = 8.6, 2.8 Hz, |
| | | 1H), 4.47 (ddd, J = 11.8, 5.6, 3.6 Hz, 1H), 4.09-4.23 (m, 1H), |
| | | 3.16-3.28 (m, 1H), 2.89-3.03 (m, 1H) |
| 41 | 300 MHz | 8.56 (d, J = 1.8 Hz, 1H), 8.24 (dd, J = 4.7, 1.5 Hz, 1H), 7.91 (d, J = 1.8 Hz, |
| | d₄-MeOH | 1H), 7.72 (d, J = 8.5 Hz, 2H), 7.46-7.64 (m, 4H), 4.48 (ddd, J = 11.7, |
| | | 6.5, 3.4 Hz, 1H), 4.16 (ddd, J = 11.8, 9.1, 2.9 Hz, 1H), 3.21-3.30 (m, |
| | | 1H), 2.88 (ddd, J = 14.5, 6.5, 2.9 Hz, 1H) |
| 42 | 300 MHz | 8.32 (dd, J = 5.0, 1.3 Hz, 1H), 8.18 (d, J = 1.5 Hz, 1H), 7.80 (dd, J = 8.5, |
| | d₄-MeOH | 1.3 Hz, 1H), 7.62-7.76 (m, 4H), 7.55 (d, J = 8.3 Hz, 2H), 7.45 (d, J = 8.6 Hz, |
| | | 1H), 7.27 (d, J = 3.2 Hz, 1H), 6.56 (d, J = 2.8 Hz, 1H), 4.50-4.67 (m, |
| | | 1H), 4.26 (ddd, J = 11.8, 8.6, 2.9 Hz, 1H), 3.84 (s, 3H), 3.17 (ddd, J = 14.6, |
| | | 8.5, 3.2 Hz, 1H), 2.87 (ddd, J = 14.7, 7.1, 2.9 Hz, 1H) |
| 43 | 300 MHz | 8.72-8.82 (m, 1H), 8.19-8.34 (m, 2H), 8.08-8.19 (m, 1H), 7.68-7.83 (m, |
| | d₄-MeOH | 3H), 7.64 (dd, J = 8.5, 5.0 Hz, 1H), 7.52 (d, J = 8.2 Hz, 2H), 4.54 (td, J = 7.7, |
| | | 3.5 Hz, 1H), 4.16-4.32 (m, 1H), 3.19 (ddd, J = 14.4, 8.1, 2.8 Hz, |
| | | 1H), 2.79 (td, J = 7.2, 4.5 Hz, 1H), 2.20 (s, 3H) |
| 44 | 300 MHz | 8.39 (s, 1H), 8.30 (d, J = 4.1 Hz, 1H), 8.13 (s, 1H), 7.86-7.97 (m, 1H), |
| | d₄-MeOH | 7.79 (d, J = 7.7 Hz, 1H), 7.59-7.76 (m, 4H), 7.56 (d, J = 8.3 Hz, 2H), |
| | | 4.51-4.66 (m, 1H), 4.27 (ddd, J = 11.6, 8.4, 2.8 Hz, 1H), 4.10 (s, 3H), |
| | | 3.19 (ddd, J = 14.5, 8.2, 3.0 Hz, 1H), 2.85 (ddd, J = 14.6, 7.3, 2.9 Hz, |
| | | 1H) |
| 45 | 300 MHz | 8.26 (dd, J = 4.5, 1.5 Hz, 1H), 8.01 (dd, J = 6.5, 1.1 Hz, 2H), 7.68 (d, J = 8.5 Hz, |
| | d₄-MeOH | 2H), 7.45-7.61 (m, 4H), 4.45 (dt, J = 11.9, 4.4 Hz, 1H), |
| | | 4.03-4.20 (m, 1H), 3.01-3.22 (m, 2H) |
| 46 | 300 MHz | 8.74-8.85 (m, 2H), 8.23 (dd, J = 4.6, 1.5 Hz, 1H), 7.97-8.07 (m, 2H), |
| | d₄-MeOH | 7.70 (d, J = 8.3 Hz, 2H), 7.52-7.60 (m, 3H), 7.42-7.52 (m, 1H), |
| | | 4.47 (ddd, J = 11.8, 6.0, 3.5 Hz, 1H), 4.12 (ddd, J = 11.9, 9.5, 2.7 Hz, 1H), |
| | | 3.23-3.39 (m, 1H), 2.89 (ddd, J = 14.4, 6.0, 2.7 Hz, 1H) |
| 47 | 300 MHz | 8.28 (dd, J = 4.6, 1.5 Hz, 1H), 8.10 (d, J = 9.2 Hz, 1H), 7.64-7.76 (m, |
| | d₄-MeOH | 2H), 7.55-7.64 (m, 3H), 7.47-7.55 (m, 1H), 7.27 (d, J = 9.2 Hz, 1H), |
| | | 4.49 (dt, J = 12.1, 4.5 Hz, 1H), 4.06-4.24 (m, 4H), 3.10-3.21 (m, 2H) |
| 48 | 300 MHz | 9.13 (d, J = 1.6 Hz, 1H), 8.82 (dd, J = 5.3, 1.5 Hz, 1H), 8.56 (dt, J = 8.2, |
| | d₄-MeOH | 1.8 Hz, 1H), 8.27 (dd, J = 4.9, 1.4 Hz, 1H), 7.79 (dd, J = 7.8, 5.6 Hz, |
| | | 1H), 7.67-7.76 (m, 3H), 7.60 (dd, J = 8.6, 4.9 Hz, 1H), 7.54 (d, J = 8.3 Hz, |
| | | 2H), 4.53 (ddd, J = 11.9, 6.8, 3.4 Hz, 1H), 4.19 (ddd, J = 11.9, 8.9, |
| | | 2.8 Hz, 1H), 3.19-3.29 (m, 1H), 2.69-2.88 (m, 1H) |
| 49 | 300 MHz | 8.49 (dd, J = 7.1, 0.8 Hz, 1H), 8.19 (dd, J = 4.1, 1.9 Hz, 1H), 8.07 (s, |
| | d₄-MeOH | 1H), 7.95 (s, 1H), 7.63-7.75 (m, 3H), 7.54-7.63 (m, 2H), 7.30-7.39 (m, |
| | | 2H), 7.23-7.30 (m, 1H), 4.33-4.48 (m, 1H), 4.06 (td, J = 11.1, 2.6 Hz, |
| | | 1H), 3.40 (ddd, J = 14.3, 10.5, 3.6 Hz, 1H), 2.94-3.07 (m, 1H) |
| 50 | 300 MHz | 8.29 (dd, J = 4.7, 1.5 Hz, 1H), 8.02 (dd, J = 5.3, 1.1 Hz, 2H), |
| | d₄-MeOH | 7.61-7.69 (m, 1H), 7.57 (dd, J = 8.6, 4.8 Hz, 1H), 7.35-7.48 (m, 2H), 7.14-7.26 (m, |
| | | 1H), 4.48 (dt, J = 12.0, 4.7 Hz, 1H), 4.17 (dt, J = 12.4, 6.1 Hz, 1H), |
| | | 3.07 (t, J = 5.5 Hz, 2H) |
| 51 | 300 MHz | 8.30 (dd, J = 4.8, 1.5 Hz, 1H), 8.17 (d, J = 2.6 Hz, 1H), 7.92 (d, J = 8.6 Hz, |
| | d₄-MeOH | 1H), 7.71 (dd, J = 8.5, 1.5 Hz, 1H), 7.61 (dd, J = 8.5, 4.8 Hz, 1H), |
| | | 7.36-7.50 (m, 2H), 7.16-7.33 (m, 2H), 4.50 (ddd, J = 11.9, 6.2, 3.5 Hz, |
| | | 1H), 4.20 (ddd, J = 12.0, 9.2, 2.9 Hz, 1H), 3.15 (ddd, J = 14.5, 9.4, 3.4 Hz, |
| | | 1H), 2.91-3.03 (m, 1H) |
| 52 | 300 MHz | 9.29-9.39 (m, 1H), 8.24-8.32 (m, 2H), 8.22 (dd, J = 4.1, 1.9 Hz, 1H), |
| | d₄-MeOH | 8.12 (d, J = 2.2 Hz, 1H), 7.97 (d, J = 9.4 Hz, 1H), 7.50 (dd, J = 11.8, 2.3 Hz, |
| | | 1H), 7.36-7.47 (m, 4H), 7.17-7.27 (m, 1H), 4.39-4.49 (m, 1H), |
| | | 4.02-4.17 (m, 1H), 3.38-3.48 (m, 1H), 2.88-3.03 (m, 1H) |
| 53 | 300 MHz | 8.31 (d, J = 0.9 Hz, 1H), 8.22 (dd, J = 3.8, 2.0 Hz, 1H), 8.13 (d, J = 0.7 Hz, |
| | d₄-MeOH | 1H), 7.86 (dd, J = 8.9, 1.6 Hz, 1H), 7.61 (d, J = 8.9 Hz, 1H), |
| | | 7.48 (dd, J = 12.0, 2.3 Hz, 1H), 7.31-7.44 (m, 3H), 7.24-7.31 (m, 1H), |
| | | 4.35-4.51 (m, 1H), 4.03-4.22 (m, 4H), 3.34-3.40 (m, 1H), 3.05-3.17 (m, 1H) |
| 54 | 300 MHz, | 10.28 (s, 1H), 8.67 (s, 1H), 8.13 (dd, J = 4.2, 1.8 Hz, 1H), 7.69 (s, 1H), |
| | DMSO-d₆ | 7.64 (dd, J = 8.3, 2.0 Hz, 1H), 7.43-7.55 (m, 2H), 7.26-7.36 (m, 2H), |
| | | 7.22 (d, J = 8.8 Hz, 1H), 6.86 (d, J = 8.2 Hz, 1H), 4.30-4.38 (m, 1H), |
| | | 4.10-4.19 (m, 1H), 3.15-3.31 (m, 1H), 2.84-2.98 (m, 3H), 2.41-2.46 (m, |
| | | 2H) |
| 55 | 400 MHz | 8.50 (d, J = 2.7 Hz, 1H), 8.25 (t, J = 2.9 Hz, 1H), 8.14 (dd, J = 8.9, 4.4 Hz, |
| | d₄-MeOH | 1H), 7.74 (td, J = 8.6, 2.8 Hz, 1H), 7.44 (dd, J = 11.9, 2.3 Hz, 1H), |
| | | 7.33-7.41 (m, 3H), 7.21-7.27 (m, 1H), 4.40 (dt, J = 11.9, 4.1 Hz, 1H), |
| | | 4.06 (td, J = 11.6, 2.3 Hz, 1H), 3.35 (m, 1H), 3.10 (ddd, J = 14.7, 11.2, |
| | | 3.8 Hz, 1H) |
| 56 | 300 MHz | 9.07 (dd, J = 4.7, 1.5 Hz, 1H), 8.70 (d, J = 8.3 Hz, 1H), 8.57 (s, 1H), |
| | d₄-MeOH | 8.28 (dd, J = 4.8, 1.4 Hz, 1H), 8.18 (d, J = 8.6 Hz, 1H), 8.08-8.15 (m, |
| | | 1H), 7.81 (dd, J = 8.3, 4.7 Hz, 1H), 7.69-7.78 (m, 2H), 7.50-7.69 (m, |
| | | 4H), 4.55 (ddd, J = 11.8, 6.6, 3.4 Hz, 1H), 4.22 (ddd, J = 11.9, 9.0, 2.8 Hz, |
| | | 1H), 3.21-3.28 (m, 1H), 2.88-3.02 (m, 1H) |
| 57 | 300 MHz | 8.19 (dd, J = 3.7, 2.2 Hz, 1H), 7.58 (d, J = 8.3 Hz, 1H), 7.50 (br. s., 1H), |
| | d₄-MeOH | 7.43 (dd, J = 12.1, 2.3 Hz, 1H), 7.36 (d, J = 7.6 Hz, 1H), 7.30-7.34 (m, |
| | | 2H), 7.23 (t, J = 7.7 Hz, 2H), 4.33-4.48 (m, 1H), 4.01-4.16 (m, 1H), |
| | | 3.19-3.29 (m, 1H), 2.99-3.13 (m, 1H) |
| 58 | 300 MHz | 8.16 (dd, J = 4.0, 2.0 Hz, 1H), 8.04 (d, J = 2.3 Hz, 1H), 7.91 (dd, J = 9.6, |
| | d₄-MeOH | 2.5 Hz, 1H), 7.37-7.46 (m, 2H), 7.21-7.35 (m, 4H), 6.49 (d, J = 9.5 Hz, |
| | | 1H), 4.34 (dt, J = 11.8, 4.1 Hz, 1H), 3.97 (td, J = 11.4, 2.3 Hz, 1H), |
| | | 3.41 (ddd, J = 14.4, 11.0, 3.6 Hz, 1H), 2.76-2.88 (m, 1H) |
| 59 | 400 MHz, | 11.93 (br. s., 1H), 8.63 (s, 1H), 7.98-8.24 (m, 2H), 7.78 (dd, J = 2.74, 9.8 Hz, |
| | DMSO-d₆ | 1H), 7.41-7.58 (m, 2H), 7.20-7.34 (m, 2H), 7.08-7.20 (m, 1H), |
| | | 6.30 (d, J = 9.8 Hz, 1H), 4.21-4.39 (m, 1H), 3.99-4.14 (m, 1H), 3.25 (dd, J = 2.7, |
| | | 7.4 Hz, 1H), 2.69-2.80 (m, 1H). |
| 60 | 300 MHz, | 8.27-8.40 (m, 2H), 7.92-8.06 (m, 1H), 7.82 (dd, J = 9.6, 2.3 Hz, 1H), |
| | CDCl₃ | 7.43-7.63 (m, 4H), 7.17-7.38 (m, 3H), 6.54 (d, J = 9.6 Hz, 1H), 4.39 (dt, |
| | | J = 11.5, 3.5 Hz, 1H), 3.90-4.11 (m, 1H), 3.61-3.79 (m, 1H), |
| | | 2.72-2.96 (m, 1H) |
| 61 | 400 MHz, | 8.83 (s, 1H), 8.11 (dd, J = 1.47, 4.21 Hz, 1H), 7.62-7.72 (m, 2H), |
| | DMSO-d₆ | 7.49-7.60 (m, 3H), 7.47 (d, J = 1.37 Hz, 1H), 7.04-7.36 (m, 3H), 4.34 (ddd, J = 2.74, |
| | | 8.02, 11.15 Hz, 1H), 4.13 (ddd, J = 2.74, 7.73, 11.05 Hz, 1H), |
| | | 3.23-3.25 (m, 1H), 2.89 (ddd, J = 2.64, 7.78, 14.23 Hz, 1H). |
| 62 | 400 MHz, | 9.61 (s, 1H), 9.42 (d, J = 2.2 Hz, 1H), 8.78 (dd, J = 8.6, 2.5 Hz, 1H), |
| | DMSO-d₆ | 8.32 (dd, J = 3.6, 2.2 Hz, 1H), 8.28 (d, J = 8.8 Hz, 1H), 7.61 (dd, J = 12.0, |
| | | 2.2 Hz, 1H), 7.54 (t, J = 8.2 Hz, 1H), 7.36-7.45 (m, 2H), 7.28 (d, J = 8.8 Hz, |
| | | 1H), 4.36-4.53 (m, 1H), 3.98-4.16 (m, 1H), 3.50 (d, J = 16.4 Hz, |
| | | 1H), 2.84-3.02 (m, 1H) |
| 63 | 400 MHz, | 8.99 (d, J = 1.6 Hz, 1H), 8.32 (dd, J = 8.0, 2.2 Hz, 1H), 8.21 (dd, J = 4.1, |
| | d₄-MeOH | 1.8 Hz, 1H), 8.15 (d, J = 8.0 Hz, 1H), 7.30-7.52 (m, 4H), 7.24 (d, J = 8.6 Hz, |
| | | 1H), 4.34-4.53 (m, 1H), 3.98-4.17 (m, 1H), 3.36-3.49 (m, |
| | | 1H), 2.88-3.06 (m, 4H) |
| 64 | 400 MHz, | 8.27 (dd, J = 4.8, 1.3 Hz, 1H), 7.60-7.69 (m, 3H), 7.51-7.58 (m, 1H), |
| | d₄-MeOH | 7.42-7.48 (m, 2H), 7.24 (d, J = 8.4 Hz, 2H), 4.45-4.59 (m, 1H), |
| | | 4.16-4.35 (m, 1H), 3.07-3.27 (m, 1H), 2.84-3.05 (m, 1H) |
| 65 | 400 MHz, | 8.48 (d, J = 2.3 Hz, 1H), 8.30 (dd, J = 4.5, 1.4 Hz, 1H), 8.05 (d, J = 8.6 Hz, |
| | d₄-MeOH | 1H), 7.83 (dd, J = 8.6, 2.5 Hz, 1H), 7.37-7.62 (m, 4H), 7.25 (d, J = 8.6 Hz, |
| | | 1H), 4.47 (dt, J = 11.9, 4.4 Hz, 1H), 4.05-4.26 (m, 1H), |
| | | 3.12-3.25 (m, 2H), 3.10 (s, 3H) |
| 66 | 400 MHz, | 8.29 (dd, J = 4.8, 1.3 Hz, 1H), 8.15 (s, 1H), 7.97 (ddd, J = 9.1, 6.7, 2.7 Hz, |
| | d₄-MeOH | 1H), 7.60-7.67 (m, 1H), 7.52-7.58 (m, 1H), 7.35-7.49 (m, 2H), |
| | | 7.20 (d, J = 8.6 Hz, 1H), 6.99 (dd, J = 9.0, 2.9 Hz, 1H), 4.48 (ddd, J = 11.8, |
| | | 6.2, 3.5 Hz, 1H), 4.18 (ddd, J = 11.9, 9.5, 2.6 Hz, 1H), 3.04-3.20 (m, |
| | | 1H), 2.78-2.90 (m, 1H). |
| 67 | 300 MHz, | 8.18-8.29 (m, 1H), 7.64-7.71 (m, J = 8.2 Hz, 2H), 7.59 (br. s., 1H), |
| | d₄-MeOH | 7.47-7.56 (m, 1H), 7.34-7.47 (m, J = 8.3 Hz, 2H), 4.25-4.46 (m, 2H), |
| | | 4.06 (ddd, J = 11.9, 9.6, 2.6 Hz, 1H), 3.02-3.20 (m, 1H), 2.54-2.72 (m, 1H), |
| | | 1.19-1.30 (m, 3H) |
| 68 | 400 MHz, | 8.28 (dd, J = 4.7, 1.4 Hz, 1H), 7.56-7.64 (m, 3H), 7.49-7.55 (m, 3H), |
| | d₄-MeOH | 7.35-7.48 (m, 2H), 7.19 (d, J = 8.8 Hz, 1H), 4.40-4.54 (m, 1H), |
| | | 4.09-4.23 (m, 1H), 3.13 (d, J = 3.7 Hz, 1H), 2.86 (dd, J = 5.9, 2.5 Hz, 1H) |
| 69 | 400 MHz, | 8.34 (d, J = 2.5 Hz, 1H), 8.26 (dd, J = 4.7, 1.6 Hz, 1H), 7.87 (dd, J = 8.7, |
| | d₄-MeOH | 2.8 Hz, 1H), 7.55-7.62 (m, 1H), 7.47-7.54 (m, 1H), 7.36-7.46 (m, 2H), |
| | | 7.32 (d, J = 8.8 Hz, 1H), 7.14-7.22 (m, 1H), 4.45 (ddd, J = 11.7, 6.1, 3.5 Hz, |
| | | 1H), 4.14 (ddd, J = 12.0, 9.5, 2.7 Hz, 1H), 3.03-3.19 (m, 1H), |
| | | 2.78-2.93 (m, 1H) |
| 70 | 300 MHz, | 8.48 (d, J = 2.5 Hz, 1H), 8.13 (dd, J = 4.8, 1.3 Hz, 1H), 7.91 (ddd, J = 8.4, |
| | d₄-MeOH | 2.6, 1.4 Hz, 1H), 7.61-7.71 (m, J = 8.5 Hz, 2H), 7.50-7.61 (m, J = 8.3 Hz, |
| | | 2H), 7.31 (dd, J = 8.2, 5.0 Hz, 1H), 7.19 (ddd, J = 8.4, 6.8, 2.0 Hz, |
| | | 1H), 6.75-6.94 (m, 3H), 4.35 (dd, J = 9.4, 2.4 Hz, 1H), 4.13-4.25 (m, |
| | | 1H), 3.34-3.39 (m, 1H), 2.40-2.59 (m, 1H) |
| 71 | 300 MHz, | 11.35 (br. s., 1H), 9.71 (s, 1H), 9.55 (s, 1H), 8.78 (d, J = 5.1 Hz, 1H), |
| | CDCl₃ | 8.28-8.45 (m, 2H), 8.18 (d, J = 8.8 Hz, 1H), 7.91 (dd, J = 7.9, 5.6 Hz, |
| | | 1H), 7.74 (dd, J = 8.5, 5.6 Hz, 1H), 7.53-7.67 (m, J = 8.3 Hz, 2H), |
| | | 6.96-7.09 (m, J = 8.0 Hz, 2H), 3.01-3.30 (m, 2H), 2.72-2.94 (m, 1H), |
| | | 2.27-2.46 (m, 1H), 1.95-2.15 (m, 1H), 1.62 (d, J = 8.9 Hz, 1H) |
| 72 | 300 MHz, | 8.12-8.24 (m, 1H), 7.59-7.72 (m, J = 8.3 Hz, 2H), 7.43-7.56 (m, J = 8.3 Hz, |
| | d₄-MeOH | 2H), 7.28-7.38 (m, 2H), 7.12-7.28 (m, 2H), 6.94 (d, J = 7.9 Hz, 1H), |
| | | 6.57-6.72 (m, 1H), 4.29-4.43 (m, 1H), 3.92-4.13 (m, 1H), 3.24-3.30 (m, |
| | | 1 H), 2.74-2.88 (m, 1H) |
| 73 | 300 MHz, | 8.47 (br. s., 1H), 8.19 (dd, J = 3.9, 2.0 Hz, 1H), 8.11 (d, J = 3.9 Hz, 1H), |
| | d₄-MeOH | 7.81-7.91 (m, 1H), 7.61-7.70 (m, J = 8.3 Hz, 2H), 7.47-7.55 (m, J = 8.5 Hz, |
| | | 2H), 7.23-7.39 (m, 3H), 4.31-4.43 (m, 1H), 4.03 (t, J = 9.8 Hz, 1H), |
| | | 3.25 (s, 1H), 2.77-2.93 (m, 1H) |
| 74 | 300 MHz, | 8.17 (dd, J = 4.1, 1.9 Hz, 1H), 7.59-7.71 (m, J = 8.3 Hz, 2H), |
| | d₄-MeOH | 7.45-7.55 (m, J = 8.3 Hz, 2H), 7.29-7.37 (m, 2H), 7.21-7.29 (m, 2H), 6.89-7.01 (m, |
| | | 2H), 4.30-4.44 (m, 1H), 4.04 (t, J = 9.6 Hz, 1H), 3.24-3.30 (m, 1 H), |
| | | 2.81 (d, J = 5.4 Hz, 1H) |
| 75 | 400 MHz, | 13.72 (br. s., 1H), 9.61 (s, 1H), 9.10 (dd, J = 2.0, 0.8 Hz, 1H), 8.47 (dd, J = 8.0, |
| | DMSO-d₆ | 2.2 Hz, 1H), 8.33 (dd, J = 3.6, 2.2 Hz, 1H), 8.10-8.23 (m, 1H), |
| | | 7.48-7.69 (m, 2H), 7.36-7.48 (m, 2H), 7.29 (dt, J = 8.8, 1.1 Hz, 1H), |
| | | 4.42 (dt, J = 11.9, 4.1 Hz, 1H), 4.07 (td, J = 11.3, 2.2 Hz, 1H), 3.50 (dt, J = 12.2, |
| | | 2.3 Hz, 1H), 2.95 (ddd, J = 14.6, 10.9, 3.6 Hz, 1H) |
| 76 | 400 MHz, | 8.10 (dd, J = 4.5, 1.4 Hz, 1H), 7.56-7.65 (m, 2H), 7.43-7.51 (m, 1H), |
| | d₄-MeOH | 7.25-7.40 (m, 4H), 7.20-7.25 (m, 1H), 7.10-7.19 (m, 2H), 4.19-4.46 (m, |
| | | 1H), 3.87-4.10 (m, 1H), 2.70-2.98 (m, 2H) |
| 77 | 400 MHz, | 8.70 (d, J = 1.8 Hz, 1H), 8.62 (dd, J = 5.0, 1.5 Hz, 1H), 7.97-8.07 (m, |
| | d₄-MeOH | 2H), 7.44 (dd, J = 8.0, 4.9 Hz, 1H), 7.30-7.38 (m, 2H), 7.16-7.29 (m, |
| | | 3H), 4.34-4.46 (m, 1H), 3.97-4.13 (m, 1H), 2.73-3.01 (m, 2H) |
| 78 | 300 MHz, | 8.22 (t, J = 3.0 Hz, 1H), 7.15-7.25 (m, 4H), 7.04-7.14 (m, 1H), 6.32 (br. |
| | CDCl₃ | s., 1H), 4.34 (dt, J = 11.7, 4.1 Hz, 1H), 4.00 (td, J = 11.4, 2.4 Hz, 1H), |
| | | 3.13 (d, J = 14.6 Hz, 1H), 2.97 (ddd, J = 14.7, 11.1, 3.8 Hz, 1H), |
| | | 1.33-1.49 (m, 9H) |
| 79 | 400 MHz, | 8.33 (m, 2H), 8.1 (s, 1H), 7.60 (dd, J = 5.2, 8.8 Hz, 1H), 7.5-7.6 (s, 4H), |
| | CDCl₃ | 7.3 (m, 2H), 6.85 (s, 1H), 6.6-6.5 (d, J = 5.6 Hz, 1H), 4.4 (m, 1H), |
| | | 4.07-4.11 (m, 1H), 3.8 (m, 1H), 3.6 (s, 1H), 2.8 (m, 1H) |
| 80 | 400 MHz, | 8.6 (s, 1H), 8.3 (m, 1H), 7.8-8.0 (m, 3H), 7.5 (s, 3H), 7.3 (m, 2H), |
| | CDCl₃ | 6.45 (s, 1H), 4.5 (s, 2H), 4.4 (m, 1H), 4.0 (m, 1H), 3.8 (m, 1H), 2.8-2.9 (m, |
| | | 1H) |
| 81 | 400 MHz, | 8.33 (dd, J = 5.2, 1.3 Hz, 1H), 8.18 (d, J = 2.5 Hz, 1H), 7.96-8.06 (m, |
| | d₄-MeOH | 2H), 7.86-7.94 (m, 2H), 7.79-7.84 (m, 1H), 7.74 (dd, J = 8.4, 5.1 Hz, |
| | | 1H), 7.51-7.59 (m, 4H), 6.55 (d, J = 9.6 Hz, 1H), 4.43-4.66 (m, 1H), |
| | | 4.19 (ddd, J = 11.9, 9.4, 2.7 Hz, 1H), 3.21-3.30 (m, 1H), 2.71-2.87 (m, |
| | | 1H) |
| 82 | 300 MHz, | 8.58 (d, J = 1.3 Hz, 1H), 8.27 (dd, J = 4.7, 1.5 Hz, 1H), 8.16-8.21 (m, |
| | MeOH-d₄ | 1H), 8.11-8.16 (m, 1H), 7.60 (dd, J = 8.5, 1.5 Hz, 1H), 7.52 (dd, J = 8.5, |
| | | 4.8 Hz, 1H), 7.36-7.49 (m, 2H), 7.17-7.26 (m, 1H), 4.45 (ddd, J = 11.9, |
| | | 5.7, 3.5 Hz, 1H), 4.14 (ddd, J = 12.1, 9.9, 2.6 Hz, 1H), 3.21 (ddd, J = 14.2, |
| | | 10.2, 3.5 Hz, 1H), 2.73-2.91 (m, 1H) |
| 83 | 300 MHz, | 8.55 (d, J = 5.0 Hz, 2H), 8.28 (dd, J = 4.9, 1.4 Hz, 1H), 7.68 (dd, J = 8.6, |
| | MeOH-d₄ | 1.4 Hz, 1H), 7.58 (dd, J = 8.6, 4.9 Hz, 1H), 7.43-7.51 (m, 1H), |
| | | 7.41 (dd, J = 11.7, 2.3 Hz, 1H), 7.17-7.26 (m, 1H), 7.06 (t, J = 4.9 Hz, 1H), |
| | | 4.40-4.58 (m, 1H), 4.16 (ddd, J = 12.2, 9.9, 2.6 Hz, 1H), 3.23 (ddd, J = 14.3, |
| | | 10.2, 3.6 Hz, 1H), 2.70-2.88 (m, 1H) |
| 84 | 300 MHz, | 8.74 (d, J = 1.6 Hz, 1H), 8.27 (dd, J = 4.7, 1.5 Hz, 1H), 8.21 (dd, J = 8.8, |
| | MeOH-d₄ | 2.3 Hz, 1H), 7.55-7.63 (m, 1H), 7.35-7.54 (m, 3H), |
| | | 7.13-7.27 (m, 2H), 4.35-4.54 (m, 1H), 4.05-4.23 (m, 1H), 3.90 (s, 3H), |
| | | 3.16-3.27 (m, 1H), 2.73-2.92 (m, 1H) |
| 85 | 300 MHz, | 8.86 (br. s., 2H), 8.77 (br. s., 1H), 8.29 (dd, J = 4.8, 1.5 Hz, 1H), |
| | MeOH-d₄ | 7.69 (dd, J = 8.5, 1.5 Hz, 1H), 7.59 (dd, J = 8.6, 4.9 Hz, 1H), 7.35-7.50 (m, |
| | | 2H), 7.13-7.23 (m, 1H), 4.49 (ddd, J = 11.9, 6.5, 3.4 Hz, 1H), |
| | | 4.18 (ddd, J = 11.9, 9.1, 2.9 Hz, 1H), 2.96-3.12 (m, 1H), 2.82 (ddd, J = 14.7, |
| | | 6.4, 2.8 Hz, 1H) |
| 86 | 300 MHz, | 8.18 (dd, J = 4.4, 1.2 Hz, 1H), 8.07 (d, J = 8.3 Hz, 1H), 7.95 (dd, J = 8.0, |
| | MeOH-d₄ | 1.5 Hz, 1H), 7.29-7.52 (m, 5H), 7.17 (d, J = 8.6 Hz, 1H), 7.01 (t, J = 7.6 Hz, |
| | | 1H), 4.33-4.51 (m, 1H), 4.14 (ddd, J = 11.5, 8.7, 2.7 Hz, 1H), |
| | | 3.89 (s, 3H), 3.27 (dd, J = 8.6, 3.2 Hz, 1H), 2.70 (ddd, J = 14.4, 6.9, 2.7 Hz, |
| | | 1H) |
| 87 | 300 MHz, | 8.67 (t, J = 1.5 Hz, 1H), 8.28 (dd, J = 4.8, 1.5 Hz, 1H), 8.07 (d, J = 1.5 Hz, |
| | MeOH-d₄ | 2H), 7.63 (dd, J = 8.5, 1.5 Hz, 1H), 7.55 (dd, J = 8.5, 4.8 Hz, 1H), |
| | | 7.36-7.48 (m, 2H), 7.15-7.23 (m, 1H), 4.47 (ddd, J = 11.9, 6.0, 3.5 Hz, |
| | | 1H), 4.15 (ddd, J = 12.0, 9.5, 2.8 Hz, 1H), 3.94 (s, 3H), 3.00-3.18 (m, |
| | | 1H), 2.77-2.93 (m, 1H) |
| 88 | 300 MHz, | 8.23-8.43 (m, 2H), 7.71-7.85 (m, 2H), 7.60-7.71 (m, 1H), 7.29-7.53 (m, |
| | MeOH-d₄ | 3H), 7.16 (d, J = 8.8 Hz, 1H), 4.41-4.58 (m, 1H), 4.20 (ddd, J = 11.8, |
| | | 9.0, 2.6 Hz, 1H), 2.92-3.12 (m, 1H), 2.62-2.82 (m, 1H) |
| 89 | 300 MHz, | 8.84 (t, J = 1.0 Hz, 1H), 8.50 (dd, J = 6.5, 0.9 Hz, 1H), 8.26 (dd, J = 4.4, |
| | MeOH-d₄ | 1.8 Hz, 1H), 7.69 (d, J = 6.6 Hz, 1H), 7.36-7.54 (m, 4H), 7.15-7.32 (m, |
| | | 2H), 4.35-4.49 (m, 1H), 4.09 (ddd, J = 11.9, 10.6, 2.6 Hz, 1H), |
| | | 3.16 (ddd, J = 14.4, 10.6, 3.7 Hz, 1H), 2.88-3.02 (m, 1H) |
| 90 | 300 MHz, | 8.32 (dd, J = 5.0, 1.5 Hz, 1H), 7.85-7.95 (m, 2H), 7.76 (dd, J = 8.6, 1.4 Hz, |
| | MeOH-d₄ | 1H), 7.65 (dd, J = 8.6, 5.0 Hz, 1H), 7.42-7.52 (m, 3H), 7.39 (dd, J = 11.5, |
| | | 2.3 Hz, 1H), 7.14-7.22 (m, 1H), 4.51 (ddd, J = 11.9, 6.2, 3.4 Hz, |
| | | 1H), 4.19 (ddd, J = 11.9, 9.2, 2.6 Hz, 1H), 3.87 (s, 3H), 3.10 (ddd, J = 14.5, |
| | | 9.4, 3.4 Hz, 1H), 2.67-2.83 (m, 1H) |
| 91 | 300 MHz, | 8.29 (dd, J = 4.8, 1.5 Hz, 1H), 8.02 (t, J = 1.8 Hz, 1H), 7.50-7.73 (m, |
| | MeOH-d₄ | 4H), 7.40-7.49 (m, 1H), 7.29-7.40 (m, 2H), 7.09-7.22 (m, 1H), |
| | | 4.48 (ddd, J = 11.8, 6.3, 3.5 Hz, 1H), 4.16 (ddd, J = 11.9, 9.4, 2.7 Hz, 1H), |
| | | 3.87 (s, 3H), 3.03-3.19 (m, 1H), 2.67-2.83 (m, 1H) |
| 92 | 300 MHz, | 8.25 (br.s. 2H), 8.20 (dd, J = 3.5, 2.5 Hz, 1H), 7.27-7.46 (m, 6H), |
| | MeOH-d₄ | 7.12-7.25 (m, 1H), 4.27-4.48 (m, 1H), 4.06 (ddd, J = 11.7, 10.6, 2.5 Hz, 1H), |
| | | 3.20 (ddd, J = 14.4, 10.6, 3.6 Hz, 1H), 2.85-3.04 (m, 1H) |
| 93 | 300 MHz, | 8.26 (dd, J = 8.2, 1.8 Hz, 1H), 8.18 (dd, J = 3.9, 1.9 Hz, 1H), 7.95 (dd, J = 4.6, |
| | MeOH-d₄ | 1.7 Hz, 1H), 7.24-7.44 (m, 5H), 7.11-7.21 (m, 1H), 4.37 (ddd, J = 11.7, |
| | | 6.1, 3.3 Hz, 1H), 4.08 (ddd, J = 11.8, 9.5, 2.6 Hz, 1H), |
| | | 3.25-3.38 (m, 1H), 2.67-2.83 (m, 1H) |
| 94 | 300 MHz, | 8.19 (dd, J = 4.0, 2.0 Hz, 1H), 7.53 (t, J = 8.0 Hz, 1H), 7.36-7.46 (m, |
| | MeOH-d₄ | 2H), 7.26-7.36 (m, 2H), 7.14-7.22 (m, 1H), 7.08-7.14 (m, 1H), |
| | | 7.00 (d, J = 8.2 Hz, 1H), 4.27-4.42 (m, 1H), 4.06 (td, J = 11.3, 2.3 Hz, 1H), |
| | | 3.33-3.42 (m, 1H), 2.73-2.88 (m, 1H) |
| 95 | 300 MHz, | 8.18 (dd, J = 4.1, 1.9 Hz, 1H), 8.07 (d, J = 3.8 Hz, 1H), 7.57-7.72 (m, |
| | MeOH-d₄ | 1H), 7.26-7.45 (m, 4H), 7.14-7.26 (m, 1H), 7.05 (d, J = 8.3 Hz, 1H), |
| | | 6.92 (dd, J = 6.9, 5.5 Hz, 1H), 4.36 (ddd, J = 11.7, 5.7, 3.5 Hz, 1H), |
| | | 4.09 (ddd, J = 11.9, 9.9, 2.5 Hz, 1H), 3.33-3.41 (m, 1H), 2.75-2.92 (m, 1H) |
| 96 | 300 MHz, | 8.48 (br. s., 1H), 8.20 (dd, J = 3.7, 2.3 Hz, 1H), 8.12 (br. s., 1H), |
| | MeOH-d₄ | 7.79-7.94 (m, 1H), 7.23-7.46 (m, 5H), 7.13-7.23 (m, 1H), 4.29-4.47 (m, |
| | | 1H), 3.97-4.15 (m, 1H), 3.21 (ddd, J = 14.3, 10.5, 3.6 Hz, 1H), |
| | | 2.81-3.01 (m, 1H) |
| 97 | 300 MHz, | 8.32 (d, J = 4.2 Hz, 1H), 7.79 (d, J = 8.5 Hz, 1H), 7.67 (dd, J = 8.4, 5.0 Hz, |
| | MeOH-d₄ | 1H), 7.47 (t, J = 8.3 Hz, 1H), 7.32-7.42 (m, 1H), 7.16 (d, J = 8.5 Hz, |
| | | 1H), 6.86-7.03 (m, 2H), 4.42-4.57 (m, 1H), 4.11-4.27 (m, 1H), |
| | | 2.97-3.13 (m, 1H), 2.64-2.79 (m, 1H) |
| 98 | 400 MHz, | 8.10 (dd, J = 3.91, 2.15 Hz, 1H), 7.39-7.48 (m, 2H), 7.20-7.28 (m, 2H), |
| | DMSO-d₆ | 7.14 (dt, J = 8.71, 1.03 Hz, 1H), 6.86 (s, 1H), 4.25-4.34 (m, 1H), |
| | | 4.04-4.12 (m, 1H), 3.20-3.28 (m, 4H), 3.02-3.15 (m, 1H), 2.80 (ddd, J = 14.33, |
| | | 7.29, 2.84 Hz, 1H), 1.49-1.55 (m, 2H), 1.38-1.46 (m, 4H). |
| 99 | 400 MHz, | 8.09 (dd, J = 4.01, 1.86 Hz, 1H), 7.38-7.49 (m, 2H), 7.19-7.29 (m, 2H), |
| | DMSO-d₆ | 7.13 (dd, J = 8.61, 0.98 Hz, 1H), 6.97 (s, 1H), 4.23-4.32 (m, 1H), |
| | | 4.03-4.11 (m, 1H), 3.83 (d, J = 13.11 Hz, 2H), 3.60 (s, 3H), 3.14 (ddd, J = 14.13, |
| | | 8.07, 2.84 Hz, 1H), 2.69-2.84 (m, 3H), 2.46-2.56 (m, 1H), |
| | | 1.71-1.80 (m, 2H), 1.34-1.48 (m, 2H). |
| 100 | 400 MHz, | 8.09 (dd, J = 4.01, 1.86 Hz, 1H), 7.39-7.49 (m, 2H), 7.18-7.29 (m, 3H), |
| | DMSO-d₆ | 7.12 (dd, J = 8.71, 1.08 Hz, 1H), 4.28 (ddd, J = 11.15, 7.63, 2.93 Hz, |
| | | 1H), 4.04 (ddd, J = 11.35, 8.22, 2.54 Hz, 1H), 3.37-3.47 (m, 4 H), |
| | | 3.19 (ddd, J = 14.13, 8.17, 2.93 Hz, 1H), 2.66 (ddd, J = 14.18, 7.34, 2.54 Hz, |
| | | 1H), 1.81-1.96 (m, 4H). |
| 101 | 400 MHz, | 8.09 (dd, J = 4.01, 1.86 Hz, 1H), 7.39-7.49 (m, 2H), 7.18-7.29 (m, 3H), |
| | DMSO-d₆ | 7.12 (dd, J = 8.71, 1.08 Hz, 1H), 4.28 (ddd, J = 11.15, 7.63, 2.93 Hz, |
| | | 1H), 4.04 (ddd, J = 11.35, 8.22, 2.54 Hz, 1H), 3.37 3.47 (m, 4H), |
| | | 3.19 (ddd, J = 14.13, 8.17, 2.93 Hz, 1H), 2.66 (ddd, J = 14.18, 7.34, 2.54 Hz, |
| | | 1H), 1.81-1.96 (m, 4H). |
| 102 | 400 MHz, | 8.09 (dd, J = 3.91, 1.96 Hz, 1H), 7.39-7.48 (m, 2H), 7.20-7.28 (m, 2H), |
| | DMSO-d₆ | 7.13 (dd, J = 8.61, 1.17 Hz, 1H), 6.93 (s, 1H), 4.67 (d, J = 4.30 Hz, 1H), |
| | | 4.25-4.32 (m, 1H), 4.08 (ddd, J = 11.25, 8.12, 2.54 Hz, 1H), |
| | | 3.62-3.71 (m, 2H), 3.55-3.62 (m, 1H), 3.12 (ddd, J = 14.28, 7.92, 2.84 Hz, 1H), |
| | | 2.81-2.92 (m, 2H), 2.75 (ddd, J = 14.18, 7.53, 2.74 Hz, 1H), 1.66 (dd, J = 8.71, |
| | | 4.01 Hz, 2H), 1.18-1.29 (m, 2H). |
| 103 | 400 MHz, | 8.10 (dd, J = 4.11, 1.76 Hz, 1H), 7.41-7.51 (m, 2H), 7.21-7.29 (m, 2H), |
| | DMSO-d₆ | 7.19 (s, 1H), 7.14 (dd, J = 8.61, 1.17 Hz, 1H), 4.26-4.34 (m, 1H), |
| | | 4.05 (ddd, J = 11.25, 8.41, 2.64 Hz, 1H), 3.59 (t, J = 5.09 Hz, 4H), 3.20 (ddd, |
| | | J = 14.08, 8.22, 2.74 Hz, 1H), 2.71 (ddd, J = 14.23, 7.19, 2.64 Hz, 1H), |
| | | 2.33 (t, J = 6.06 Hz, 4H). |
| 104 | 400 MHz, | 8.14 (t, J = 3.03 Hz, 1H), 7.40-7.53 (m, 2H), 7.27 (d, J = 2.74 Hz, 2H), |
| | DMSO-d₆ | 7.14 (dd, J = 8.71, 1.08 Hz, 1H), 6.45 (s, 1H), 4.32 (ddd, J = 11.44, 6.06, |
| | | 3.62 Hz, 1H), 4.01 (ddd, J = 11.59, 8.56, 3.33 Hz, 1H), 3.22 (t, J = 6.46 Hz, |
| | | 4H), 2.91-3.06 (m, 2H), 1.73-1.84 (m, 4H). |
| 105 | 400 MHz, | 8.09 (dd, J = 3.91, 1.96 Hz, 1H), 7.39-7.48 (m, 2H), 7.19-7.29 (m, 2H), |
| | DMSO-d₆ | 7.12 (dd, J = 8.80, 1.17 Hz, 1H), 6.65 (s, 1 H), 4.30 (ddd, J = 11.20, |
| | | 7.87, 3.03 Hz, 1H), 4.07 (ddd, J = 11.25, 7.92, 2.74 Hz, 1H), 3.82 (t, J = 7.73 Hz, |
| | | 4H), 3.11 (ddd, J = 14.28, 7.73, 2.84 Hz, 1H), 2.69-2.81 (m, 1 |
| | | H), 2.10 (quin, J = 7.53 Hz, 2H). |
| 106 | 300 MHz, | 8.18-8.28 (m, 1H), 8.14 (d, J = 8.3 Hz, 1H), 8.00 (d, J = 7.6 Hz, 1H), |
| | MeOH-d₄ | 7.53-7.65 (m, 1H), 7.28-7.53 (m, 4H), 7.17 (d, J = 7.9 Hz, 1H), 7.00 (t, J = 7.2 Hz, |
| | | 1H), 4.37-4.54 (m, 1H), 4.17 (t, J = 9.4 Hz, 1H), 3.12-3.26 (m, |
| | | 1H), 2.55-2.76 (m, 1H) |
| 107 | 300 MHz, | 8.25 (dd, J = 4.6, 1.5 Hz, 1H), 7.98 (t, J = 1.8 Hz, 1H), 7.63 (dt, J = 7.7, |
| | MeOH-d₄ | 1.3 Hz, 1H), 7.51-7.60 (m, 2H), 7.28-7.51 (m, 4H), 7.13-7.21 (m, 1H), |
| | | 4.44 (ddd, J = 11.8, 6.1, 3.4 Hz, 1H), 4.01-4.23 (m, 1H), 3.07-3.24 (m, |
| | | 1H), 2.69-2.91 (m, 1H) |
| 108 | 300 MHz, | 8.26 (dd, J = 4.8, 1.5 Hz, 1H), 7.82-7.98 (m, 2H), 7.58 (dd, J = 8.5, 1.5 Hz, |
| | MeOH-d₄ | 1H), 7.51 (dd, J = 8.5, 4.7 Hz, 1H), 7.33-7.47 (m, 4H), 7.13-7.21 (m, |
| | | 1H), 4.45 (ddd, J = 11.8, 6.0, 3.4 Hz, 1H), 4.14 (ddd, J = 11.9, 9.5, 2.7 Hz, |
| | | 1H), 3.07-3.25 (m, 1H), 2.70-2.90 (m, 1H) |
| 109 | 300 MHz, | 9.10 (d, J = 1.6 Hz, 1H), 8.30-8.42 (m, 2H), 8.22 (d, J = 8.2 Hz, 1H), |
| | MeOH-d₄ | 8.03 (td, J = 8.5, 1.3 Hz, 1H), 7.87 (dd, J = 8.6, 5.3 Hz, 1H), |
| | | 7.41-7.54 (m, 2H), 7.24 (d, J = 8.6 Hz, 1H), 4.56-4.70 (m, 1H), 4.35-4.45 (m, 1H), |
| | | 3.02-3.18 (m, 1H), 2.79 (ddd, J = 14.7, 8.2, 3.2 Hz, 1H) |
| 110 | 400 MHz, | 12.15 (br. s., 1H), 8.10 (dd, J = 3.91, 1.96 Hz, 1H), 7.39-7.48 (m, 2H), |
| | DMSO-d₆ | 7.20-7.28 (m, 2H), 7.13 (dd, J = 8.71, 1.27 Hz, 1H), 6.93 (s, 1H), |
| | | 4.29 (ddd, J = 11.10, 7.58, 3.03 Hz, 1H), 4.07 (ddd, J = 11.25, 8.22, 2.84 Hz, |
| | | 1H), 3.82 (d, J = 13.11 Hz, 2H), 3.13 (ddd, J = 14.28, 8.22, 3.13 Hz, |
| | | 1H), 2.68-2.84 (m, 3H), 2.39 (tt, J = 11.13, 3.94 Hz, 1H), 1.70-1.80 (m, |
| | | 2H), 1.35-1.47 (m, 2H). |
| 111 | 300 MHz, | 8.72 (s, 1H), 8.12 (dd, J = 4.0, 2.0 Hz, 1H), 7.79-7.96 (m, 2H), |
| | DMSO-d₆ | 7.13-7.40 (m, 8H), 4.22-4.43 (m, 1H), 4.00 (s, 2H), 3.93-4.07 (m, 1H), |
| | | 3.19-3.28 (m, 1H), 2.82 (ddd, J = 14.1, 6.1, 2.5 Hz, 1H) |
| 112 | 300 MHz, | 8.77 (s, 1H), 8.65 (d, J = 4.8 Hz, 1H), 8.15 (dd, J = 4.0, 1.8 Hz, 1H), |
| | DMSO-d₆ | 7.96-8.08 (m, J = 8.5 Hz, 2H), 7.77-7.96 (m, 4H), 7.37-7.48 (m, J = 8.5 Hz, |
| | | 2H), 7.15-7.37 (m, 5H), 4.26-4.43 (m, 1H), 4.04 (t, J = 9.3 Hz, |
| | | 1H), 3.26-3.36 (m, 1 H), 2.79-2.96 (m, 1H) |
| 113 | 300 MHz, | 8.76 (s, 1H), 8.16 (dd, J = 4.1, 1.9 Hz, 1H), 7.81-7.99 (m, 2H), |
| | DMSO-d₆ | 7.54-7.71 (m, 4H), 7.34-7.52 (m, 4H), 7.20-7.34 (m, 4H), 4.36 (ddd, J = 11.4, |
| | | 5.5, 3.4 Hz, 1H), 3.95-4.13 (m, 1H), 3.22-3.42 (m, 1H), |
| | | 2.80-2.95 (m, 1H) |
| 114 | 300 MHz, | 8.75 (s, 1H), 8.16 (dd, J = 3.9, 1.9 Hz, 1H), 7.79-7.99 (m, 2H), |
| | DMSO-d₆ | 7.63-7.74 (m, 2H), 7.59 (d, J = 8.6 Hz, 2H), 7.39 (d, J = 8.5 Hz, 2H), |
| | | 7.20-7.34 (m, 6H), 4.36 (ddd, J = 11.4, 5.6, 3.4 Hz, 1H), 3.93-4.15 (m, 1H), |
| | | 3.20-3.42 (m, 1H), 2.80-2.98 (m, 1H) |
| 115 | 300 MHz, | 8.95 (d, J = 2.0 Hz, 1H), 8.80 (s, 1H), 8.62 (d, J = 3.7 Hz, 1H), 8.22 (d, J = 8.5 Hz, |
| | DMSO-d₆ | 1H), 8.16 (dd, J = 4.2, 1.8 Hz, 1H), 7.83-7.96 (m, 2H), |
| | | 7.72 (d, J = 8.5 Hz, 2H), 7.60 (dd, J = 7.7, 5.0 Hz, 1H), 7.47 (m, J = 8.5 Hz, |
| | | 2H), 7.19-7.38 (m, 4H), 4.29-4.45 (m, 1H), 4.08 (t, J = 8.9 Hz, 1H), |
| | | 2.83-2.97 (m, 1H) |
| 116 | 300 MHz, | 8.80 (s, 1H), 8.63 (d, J = 5.6 Hz, 2H), 8.15 (dd, J = 4.0, 1.8 Hz, 1H), |
| | DMSO-d₆ | 7.84-7.97 (m, 2H), 7.76 (d, J = 8.6 Hz, 2H), 7.69 (d, J = 6.1 Hz, 2H), |
| | | 7.47 (d, J = 8.6 Hz, 2H), 7.20-7.35 (m, 4H), 4.36 (dt, J = 11.3, 3.2 Hz, |
| | | 1H), 4.07 (t, J = 8.9 Hz, 1H), 2.81-2.98 (m, 1H) |
| 117 | 300 MHz, | 8.70 (s, 1H), 8.68 (d, J = 1.3 Hz, 1H), 8.28 (d, J = 1.5 Hz, 1H), 8.08 (dd, |
| | DMSO-d₆ | J = 4.1, 1.9 Hz, 1H), 7.78-7.91 (m, 4H), 7.35 (d, J = 8.6 Hz, 2H), |
| | | 7.13-7.27 (m, 4H), 4.21-4.38 (m, 3H), 3.97 (t, J = 9.3 Hz, 1H), |
| | | 3.19-3.28 (m, 1H), 2.74-2.88 (m, 1H), 1.30 (t, J = 7.0 Hz, 3H) |
| 118 | 500 MHz, | 8.74 (s, 1H), 8.16 (dd, J = 4.1, 1.5 Hz, 1H), 7.89 (dd, J = 8.7, 5.6 Hz, |
| | DMSO-d₆ | 2H), 7.55-7.62 (m, 3H), 7.45-7.54 (m, 1H), 7.36-7.43 (m, 3H), |
| | | 7.22-7.34 (m, 5H), 5.22 (t, J = 5.7 Hz, 1H), 4.55 (d, J = 5.7 Hz, 2H), |
| | | 4.32-4.39 (m, 1H), 4.03 (t, J = 9.6 Hz, 1H), 2.85-2.91 (m, 1H) |
| 119 | 500 MHz, | 8.79 (s, 1H), 8.15 (dd, J = 4.2, 1.4 Hz, 1H), 7.96-8.01 (m, 2H), |
| | DMSO-d₆ | 7.85-7.96 (m, 4H), 7.66-7.75 (m, J = 8.4 Hz, 2H), 7.42-7.49 (m, J = 8.4 Hz, |
| | | 2H), 7.21-7.33 (m, 4H), 4.32-4.41 (m, 1H), 4.08 (t, J = 9.1 Hz, 1H), |
| | | 3.25 (s, 3H), 2.88-2.94 (m, 1H) |
| 120 | 500 MHz, | 9.51 (br. s., 1H), 8.70 (s, 1H), 8.17 (dd, J = 4.1, 1.6 Hz, 1H), 7.89 (dd, |
| | DMSO-d₆ | J = 8.7, 5.6 Hz, 2H), 7.49 (d, J = 8.4 Hz, 2H), 7.21-7.36 (m, 7H), 7.14 (t, |
| | | J = 7.0 Hz, 1H), 6.93 (d, J = 7.9 Hz, 1H), 6.86 (t, J = 7.4 Hz, 1H), |
| | | 4.31-4.40 (m, 1H), 4.03 (t, J = 9.7 Hz, 1H), 2.85-2.97 (m, 1H) |
| 121 | 500 MHz, | 10.00 (s, 1H), 8.72 (s, 1H), 8.16 (dd, J = 4.1, 1.5 Hz, 1H), 7.89 (dd, J = 8.7, |
| | DMSO-d₆ | 5.6 Hz, 2H), 7.62-7.73 (m, 2H), 7.52-7.62 (m, 4H), 7.36 (d, J = 8.4 Hz, |
| | | 2H), 7.23-7.33 (m, 4H), 4.30-4.40 (m, 1H), 4.02 (t, J = 9.6 Hz, |
| | | 1H), 2.87 (dt, J = 11.6, 2.6 Hz, 1H), 2.05 (s, 3H) |
| 122 | 500 MHz, | 8.74 (s, 1H), 8.16 (dd, J = 4.1, 1.5 Hz, 1H), 7.89 (dd, J = 8.7, 5.6 Hz, |
| | DMSO-d₆ | 2H), 7.72 (d, J = 5.0 Hz, 1H), 7.57 (d, J = 8.4 Hz, 3H), 7.39 (d, J = 8.4 Hz |
| | | 2H), 7.17-7.33 (m, 5H), 5.31 (t, J = 5.7 Hz, 1H), 4.59 (d, J = 5.7 Hz, |
| | | 2H), 4.31-4.40 (m, 1H), 4.04 (t, J = 9.5 Hz, 1H), 2.83-2.98 (m, |
| | | 1H) |
| 123 | 500 MHz, | 8.76 (s, 1H), 8.15 (dd, J = 4.1, 1.5 Hz, 1H), 7.84-7.98 (m, 3H), |
| | DMSO-d₆ | 7.58-7.77 (m, 5H), 7.55 (d, J = 8.2 Hz, 1H), 7.42 (d, J = 8.4 Hz, 2H), |
| | | 7.23-7.34 (m, 4H), 4.30-4.41 (m, 1H), 4.05 (t, J = 9.3 Hz, 1H), |
| | | 2.80-2.93 (m, 1H) |
| 124 | 500 MHz, | 9.18 (s, 1H), 9.13 (s, 2H), 8.80 (s, 1H), 8.15 (dd, J = 4.2, 1.5 Hz, 1H), |
| | DMSO-d₆ | 7.90 (dd, J = 8.7, 5.6 Hz, 2H), 7.73-7.78 (m, J = 8.4 Hz, 2H), |
| | | 7.44-7.51 (m, J = 8.4 Hz, 2H), 7.23-7.32 (m, 4H), 4.33-4.39 (m, 1H), |
| | | 4.08 (t, J = 8.9 Hz, 1H), 2.90-2.96 (m, 1H) |
| 125 | 500 MHz, | 8.75 (s, 1H), 8.16 (dd, J = 4.1, 1.4 Hz, 1H), 7.89 (dd, J = 8.7, 5.6 Hz, |
| | DMSO-d₆ | 2H), 7.32-7.42 (m, 4H), 7.22-7.32 (m, 4H), 4.32-4.38 (m, 1H), |
| | | 4.01 (t, J = 9.6 Hz, 1H), 2.77-2.89 (m, 1H), 2.39 (s, 3H), 2.22 (s, 3H) |
| 126 | 500 MHz, | 8.72 (s, 1H), 8.16 (dd, J = 4.1, 1.6 Hz, 1H), 7.89 (dd, J = 8.7, 5.5 Hz, |
| | DMSO-d₆ | 2H), 7.49 (d, J = 8.4 Hz, 2H), 7.22-7.38 (m, 6H), 7.08 (t, J = 7.8 Hz, |
| | | 1H), 6.81 (s, 1H), 6.75 (d, J = 7.7 Hz, 1H), 6.55 (d, J = 7.9 Hz, 1H), |
| | | 5.12 (s, 2H), 4.31-4.38 (m, 1H), 4.01 (t, J = 9.9 Hz, 1H), 2.85 (dd, J = 14.1, |
| | | 2.8 Hz, 1H) |
| 127 | 500 MHz, | 8.92 (s, 2H), 8.76 (s, 1H), 8.15 (dd, J = 4.2, 1.5 Hz, 1H), 7.89 (dd, J = 8.7, |
| | DMSO-d₆ | 5.6 Hz, 2H), 7.59-7.72 (m, J = 8.4 Hz, 2H), 7.39-7.50 (m, J = 8.4 Hz, |
| | | 2H), 7.23-7.33 (m, 4H), 4.30-4.40 (m, 1H), 4.06 (t, J = 9.2 Hz, |
| | | 1H), 3.96 (s, 3H), 2.87-2.96 (m, 1H) |
| 128 | 500 MHz, | 8.75 (s, 1H), 8.17 (dd, J = 4.1, 1.5 Hz, 1H), 7.90 (dd, J = 8.6, 5.6 Hz, |
| | DMSO-d₆ | 2H), 7.56 (d, J = 7.5 Hz, 1H), 7.20-7.39 (m, 11H), 5.11 (t, J = 5.4 Hz, |
| | | 1H), 4.32-4.44 (m, 3H), 4.00 (t, J = 9.9 Hz, 1H), 2.80-2.89 (m, 1H) |
| 129 | 500 MHz, | 8.76 (s, 1H), 8.16 (dd, J = 4.2, 1.5 Hz, 1H), 7.89 (dd, J = 8.6, 5.6 Hz, |
| | DMSO-d₆ | 2H), 7.72 (d, J = 7.8 Hz, 1H), 7.60-7.67 (m, 3H), 7.52 (t, J = 7.7 Hz, |
| | | 1H), 7.41 (d, J = 8.4 Hz, 2H), 7.37 (d, J = 7.6 Hz, 1H), 7.20-7.34 (m, |
| | | 4H), 4.33-4.39 (m, 1H), 4.01-4.09 (m, 1H), 3.00 (br. s., 3H), 2.93 (br. |
| | | s., 3H), 2.87-2.90 (m, 1H) |
| 130 | 500 MHz, | 8.75 (s, 1H), 8.44 (d, J = 5.7 Hz, 1H), 8.35 (s, 1H), 8.16 (dd, J = 4.1, 1.5 Hz, |
| | DMSO-d₆ | 1H), 7.90 (dd, J = 8.6, 5.5 Hz, 2H), 7.47 (d, J = 8.4 Hz, 2H), 7.38 (d, |
| | | J = 8.4 Hz, 2H), 7.23-7.34 (m, 4H), 7.15 (d, J = 5.7 Hz, 1H), |
| | | 4.28-4.44 (m, 1H), 3.93-4.10 (m, 1H), 3.84 (s, 3H), 2.80-2.98 (m, 1H) |
| 131 | 500 MHz, | 8.75 (s, 1H), 8.13-8.18 (m, 2H), 7.90 (dd, J = 8.7, 5.6 Hz, 2H), |
| | DMSO-d₆ | 7.73 (dd, J = 7.3, 1.7 Hz, 1H), 7.51 (d, J = 8.4 Hz, 2H), 7.37 (d, J = 8.4 Hz, |
| | | 2H), 7.20-7.33 (m, 4H), 7.08 (dd, J = 7.3, 4.9 Hz, 1H), 4.31-4.40 (m, |
| | | 1H), 4.05 (t, J = 9.4 Hz, 1H), 3.87 (s, 3H), 2.86-2.92 (m, 1H) |
| 132 | 500 MHz, | 8.77 (s, 1H), 8.41 (d, J = 4.9 Hz, 1H), 8.36 (s, 1H), 8.17 (dd, J = 4.2, 1.5 Hz, |
| | DMSO-d₆ | 1H), 7.84-7.92 (m, 2H), 7.34-7.43 (m, 4H), 7.22-7.33 (m, 5H), |
| | | 4.33-4.39 (m, 1H), 4.02 (t, J = 9.8 Hz, 1H), 2.86 (dd, J = 13.9, 2.8 Hz, |
| | | 1H), 2.27 (s, 3H) |
| 133 | 500 MHz, | 8.70 (s, 1H), 8.16 (dd, J = 4.1, 1.7 Hz, 1H), 7.88 (dd, J = 8.7, 5.6 Hz, |
| | DMSO-d₆ | 2H), 7.54 (t, J = 8.3 Hz, 4H), 7.20-7.35 (m, 6H), 7.01 (d, J = 8.8 Hz, |
| | | 2H), 4.31-4.38 (m, 1H), 4.01 (t, J = 9.8 Hz, 1H), 3.68-3.81 (m, 4H), |
| | | 3.12-3.18 (m, 4H), 2.84-2.90 (m, 1H) |
| 134 | 500 MHz, | 9.58 (s, 1H), 8.72 (s, 1H), 8.16 (dd, J = 4.1, 1.5 Hz, 1H), 7.89 (dd, J = 8.7, |
| | DMSO-d₆ | 5.6 Hz, 2H), 7.44-7.52 (m, J = 8.4 Hz, 2H), 7.35 (d, J = 8.4 Hz, |
| | | 2H), 7.22-7.31 (m, 4H), 7.12-7.18 (m, 1H), 7.08 (d, J = 7.7 Hz, 1H), |
| | | 6.80-6.91 (m, 1H), 4.31-4.39 (m, 1H), 4.04 (t, J = 9.5 Hz, 1H), |
| | | 2.84-2.93 (m, 1H) |
| 135 | 500 MHz, | 8.70 (s, 1H), 8.58 (s, 1H), 8.16 (dd, J = 4.1, 1.5 Hz, 1H), 7.89 (dd, J = 8.6, |
| | DMSO-d₆ | 5.6 Hz, 2H), 7.48 (d, J = 8.4 Hz, 2H), 7.22-7.35 (m, 6H), |
| | | 6.90-6.98 (m, 1H), 6.80-6.89 (m, 2H), 4.29-4.41 (m, 1H), 4.02 (t, J = 9.7 Hz, |
| | | 1H), 3.83 (s, 3H), 2.86 (dd, J = 14.1, 2.8 Hz, 1H) |
| 136 | 500 MHz, | 8.73 (s, 1H), 8.17 (dd, J = 4.1, 1.5 Hz, 1H), 7.89 (dd, J = 8.6, 5.6 Hz, |
| | DMSO-d₆ | 2H), 7.22-7.35 (m, 8H), 7.09-7.19 (m, 2H), 6.88 (dd, J = 8.4, 2.6 Hz, |
| | | 1H), 5.13 (t, J = 5.4 Hz, 1H), 4.38 (d, J = 5.4 Hz, 2H), 4.32-4.37 (m, |
| | | 1H), 3.99 (t, J = 10.1 Hz, 1H), 3.79 (s, 3H), 2.83 (dd, J = 14.6, 2.3 Hz, |
| | | 1H) |
| 137 | 500 MHz, | 8.76 (s, 1H), 8.14-8.17 (m, 1H), 7.90 (dd, J = 8.7, 5.6 Hz, 2H), 7.71 (d, |
| | DMSO-d₆ | J = 8.2 Hz, 2H), 7.65 (d, J = 8.4 Hz, 2H), 7.48 (d, J = 8.2 Hz, 2H), |
| | | 7.41 (d, J = 8.4 Hz, 2H), 7.18-7.32 (m, 4H), 4.32-4.39 (m, 1H), 4.04 (t, J = 9.5 Hz, |
| | | 1H), 3.56 (br. s., 1H), 2.98 (br. s., 3H), 2.96 (br. s., 3H), |
| | | 2.89 (dd, J = 14.3, 3.1 Hz, 1H) |
| 138 | 500 MHz, | 8.76 (s, 1H), 8.45 (d, J = 4.4 Hz, 1H), 8.15 (dd, J = 4.1, 1.5 Hz, 1H), |
| | DMSO-d₆ | 7.85-7.92 (m, 4H), 7.74 (d, J = 8.3 Hz, 2H), 7.68 (d, J = 8.4 Hz, 2H), |
| | | 7.42 (d, J = 8.4 Hz, 2H), 7.21-7.32 (m, 4H), 4.33-4.39 (m, 1H), |
| | | 4.01-4.10 (m, 1H), 2.87-2.93 (m, 1H), 2.80 (d, J = 4.4 Hz, 3H) |
| 139 | 500 MHz, | 9.11 (s, 2H), 8.80 (s, 1H), 8.14 (dd, J = 4.2, 1.4 Hz, 1H), 7.90 (dd, J = 8.7, |
| | DMSO-d₆ | 5.6 Hz, 2H), 7.76 (d, J = 8.4 Hz, 2H), 7.49 (d, J = 8.4 Hz, 2H), |
| | | 7.21-7.32 (m, 4H), 4.33-4.40 (m, 1H), 4.10 (t, J = 8.8 Hz, 1H), |
| | | 2.90-2.97 (m, 1H) |
| 140 | 500 MHz, | 8.76 (s, 1H), 8.17 (dd, J = 4.1, 1.5 Hz, 1H), 7.90 (dd, J = 8.6, 5.6 Hz, |
| | DMSO-d₆ | 2H), 7.58 (dd, J = 8.4, 6.3 Hz, 1H), 7.36 (s, 4H), 7.23-7.33 (m, 4H), |
| | | 7.18-7.23 (m, 1H), 7.04 (dd, J = 9.8, 2.6 Hz, 1H), 5.16 (t, J = 5.3 Hz, |
| | | 1H), 4.32-4.38 (m, 2H), 4.01 (br. s., 1H), 2.86 (dd, J = 14.1, 2.9 Hz, |
| | | 1H) |
| 141 | 500 MHz, | 9.18 (d, J = 2.1 Hz, 1H), 8.99 (d, J = 1.7 Hz, 1H), 8.80 (s, 1H), 8.63 (s, |
| | DMSO-d₆ | 1H), 8.15 (dd, J = 4.1, 1.4 Hz, 1H), 7.90 (dd, J = 8.7, 5.6 Hz, 2H), |
| | | 7.77 (d, J = 8.4 Hz, 2H), 7.48 (d, J = 8.4 Hz, 2H), 7.21-7.34 (m, 4H), |
| | | 4.32-4.41 (m, 1H), 4.09 (t, J = 9.0 Hz, 1H), 2.90-2.97 (m, 1H) |
| 142 | 500 MHz, | 8.73 (s, 1H), 8.16 (dd, J = 4.1, 1.5 Hz, 1H), 7.89 (dd, J = 8.7, 5.6 Hz, |
| | DMSO-d₆ | 2H), 7.55-7.61 (m, J = 8.4 Hz, 2H), 7.34-7.39 (m, J = 8.4 Hz, 2H), |
| | | 7.21-7.33 (m, 5H), 7.13 (s, 1H), 7.06 (d, J = 7.5 Hz, 1H), |
| | | 6.89-6.96 (m, 1H), 4.32-4.39 (m, 1H), 4.03 (t, J = 9.6 Hz, 1H), 3.71-3.78 (m, |
| | | 4H), 3.14-3.19 (m, 4H), 2.89 (dd, J = 14.2, 3.0 Hz, 1H) |
| 143 | 500 MHz, | 8.68 (s, 1H), 8.17 (dd, J = 4.1, 1.6 Hz, 1H), 7.89 (dd, J = 8.6, 5.5 Hz, |
| | DMSO-d₆ | 2H), 7.33-7.42 (m, 4H), 7.24-7.33 (m, 4H), 7.03 (t, J = 7.6 Hz, 1H), |
| | | 6.98 (d, J = 7.5 Hz, 1H), 6.74 (d, J = 7.9 Hz, 1H), 6.62 (t, J = 7.4 Hz, |
| | | 1H), 4.73 (s, 2H), 4.32-4.39 (m, 1H), 3.98-4.08 (m, 1H), |
| | | 2.82-2.92 (m, 1H) |
| 144 | 500 MHz, | 8.82 (s, 1H), 8.17 (dd, J = 4.2, 1.5 Hz, 1H), 8.09 (d, J = 7.3 Hz, 1H), |
| | DMSO-d₆ | 7.82-7.94 (m, 2H), 7.75 (t, J = 7.5 Hz, 1H), 7.66 (t, J = 7.3 Hz, 1H), |
| | | 7.33-7.41 (m, 4H), 7.23-7.33 (m, 5H), 4.30-4.40 (m, 1H), 3.97 (t, J = 10.9 Hz, |
| | | 1H), 2.80-2.90 (m, 1H), 2.76 (s, 3H) |
| 145 | 500 MHz, | 8.70 (s, 1H), 8.11-8.16 (m, 2H), 7.88 (dd, J = 8.7, 5.6 Hz, 2H), 7.72 (s, |
| | DMSO-d₆ | 1H), 7.54 (d, J = 8.4 Hz, 2H), 7.21-7.34 (m, 6H), 6.91 (s, 1H), |
| | | 4.31-4.37 (m, 1H), 4.00 (t, J = 9.7 Hz, 1H), 2.82-2.88 (m, 1H) |
| 146 | 500 MHz, | 8.78 (s, 1H), 8.21 (d, J = 5.3 Hz, 1H), 8.14 (dd, J = 4.2, 1.5 Hz, 1H), |
| | DMSO-d₆ | 7.89 (dd, J = 8.7, 5.6 Hz, 2H), 7.68-7.79 (m, J = 8.4 Hz, 2H), |
| | | 7.39-7.49 (m, J = 8.4 Hz, 2H), 7.23-7.34 (m, 5H), 7.08 (s, 1H), |
| | | 4.31-4.39 (m, 1H), 4.06 (t, J = 9.1 Hz, 1H), 3.88 (s, 3H), 2.86-2.93 (m, 1H) |
| 147 | 500 MHz, | 8.67 (s, 1H), 8.16 (dd, J = 4.0, 1.7 Hz, 1H), 7.88 (dd, J = 8.7, 5.5 Hz, |
| | DMSO-d₆ | 2H), 7.47 (d, J = 8.4 Hz, 2H), 7.33 (d, J = 8.4 Hz, 2H), 7.23-7.30 (m, |
| | | 6H), 6.62 (d, J = 8.4 Hz, 2H), 5.20 (s, 2H), 4.30-4.37 (m, 1H), |
| | | 3.93-4.02 (m, 1H), 2.80-2.89 (m, 1H) |
| 148 | 500 MHz, | 8.70 (s, 1H), 8.15 (dd, J = 4.1, 1.6 Hz, 1H), 7.89 (dd, J = 8.7, 5.6 Hz, |
| | DMSO-d₆ | 2H), 7.48 (d, J = 8.3 Hz, 2H), 7.22-7.37 (m, 6H), 6.98 (d, J = 7.6 Hz, |
| | | 1H), 6.85-6.89 (m, 1H), 6.71-6.76 (m, 1H), 4.89 (s, 2H), |
| | | 4.31-4.38 (m, 1H), 4.01 (t, J = 9.8 Hz, 1H), 2.81-2.89 (m, 1H), 2.07 (s, 3H) |
| 149 | 500 MHz, | 8.74 (s, 1H), 8.36 (s, 1H), 8.15 (dd, J = 4.1, 1.5 Hz, 1H), 7.89 (dd, J = 8.7, |
| | DMSO-d₆ | 5.6 Hz, 2H), 7.48 (d, J = 8.4 Hz, 2H), 7.37 (d, J = 8.4 Hz, 2H), |
| | | 7.22-7.33 (m, 4H), 4.31-4.39 (m, 1H), 4.05 (t, J = 9.3 Hz, 1H), 3.93 (s, |
| | | 6H), 2.85-2.96 (m, 1H) |
| 150 | 500 MHz, | 8.78 (s, 1H), 8.18 (s, 1H), 8.16 (dd, J = 4.1, 1.5 Hz, 1H), 7.96 (d, J = 7.8 Hz, |
| | DMSO-d₆ | 1H), 7.83-7.93 (m, 4H), 7.69 (d, J = 8.5 Hz, 3H), 7.45 (d, J = 8.4 Hz, |
| | | 3H), 7.23-7.34 (m, 5H), 4.31-4.40 (m, 1H), 4.07 (t, J = 9.4 Hz, |
| | | 1H), 2.86-2.95 (m, 1H), 2.59 (s, 3H) |
| 151 | 500 MHz, | 8.73 (s, 1H), 8.16 (dd, J = 4.1, 1.5 Hz, 1H), 7.89 (dd, J = 8.7, 5.6 Hz, |
| | DMSO-d₆ | 2H), 7.61 (d, J = 7.9 Hz, 4H), 7.38 (dd, J = 8.1, 4.6 Hz, 4H), |
| | | 7.17-7.34 (m, 4H), 5.19 (t, J = 5.7 Hz, 1H), 4.53 (d, J = 5.7 Hz, 2H), |
| | | 4.31-4.39 (m, 1H), 3.97-4.09 (m, 1H), 2.84-2.94 (m, 1H) |
| 152 | 300 MHz, | 8.19 (dd, J = 3.9, 1.9 Hz, 1H), 7.24-7.50 (m, 4H), 7.09-7.23 (m, 1H), |
| | MeOH-d₄ | 4.46-4.59 (m, 1H), 4.14 (ddd, J = 11.5, 8.1, 3.1 Hz, 1H), 2.74 (ddd, J = 14.6, |
| | | 7.3, 3.1 Hz, 1H), 2.61 (ddd, J = 14.6, 7.9, 3.2 Hz, 1H), 1.23 (s, 9H). |
| 153 | 300 MHz, | 8.18 (dd, J = 4.0, 2.0 Hz, 1H), 7.75-7.92 (m, 6H), 7.65-7.73 (m, 2H), |
| | MeOH-d₄ | 7.43-7.51 (m, 2H), 7.28-7.38 (m, 2H), 7.11-7.23 (m, 2H), 4.38 (dt, J = 11.6, |
| | | 4.0 Hz, 1H), 3.97-4.11 (m, 1H), 3.34-3.46 (m, 1H), 2.92-3.02 (m, |
| | | 1H) |
| 154 | 300 MHz, | 8.20 (dd, J = 3.9, 2.0 Hz, 1H), 7.73-7.87 (m, 6H), 7.63-7.73 (m, 2H), |
| | MeOH-d₄ | 7.40-7.59 (m, 5H), 7.27-7.38 (m, 2H), 4.40 (dt, J = 11.7, 4.0 Hz, 1H), |
| | | 3.99-4.12 (m, 1H), 3.33-3.42 (m, 1H), 3.00-3.13 (m, 1H) |
| 155 | 300 MHz, | 11.80 (s, 1H), 8.82 (s, 1H), 8.17 (dd, J = 4.4, 1.6 Hz, 1H), 7.81-8.01 (m, |
| | DMSO-d₆ | 4H), 7.71 (d, J = 8.5 Hz, 2H), 7.61 (dd, J = 8.3, 1.8 Hz, 1H), 7.53 (d, J = 1.6 Hz, |
| | | 1H), 7.45 (d, J = 8.5 Hz, 2H), 7.27-7.41 (m, 3H), 4.31-4.48 (m, |
| | | 1H), 4.10 (t, J = 9.0 Hz, 1H), 3.19-3.40 (m, 1H), 2.78-2.97 (m, 1H). |
| 156 | 300 MHz, | 8.20 (dd, J = 3.8, 2.2 Hz, 1H), 8.01-8.09 (m, 2H), 7.85-7.95 (m, 2H), |
| | MeOH-d₄ | 7.27-7.50 (m, 4H), 7.17-7.27 (m, 1H), 4.35-4.47 (m, 1H), |
| | | 4.04-4.14 (m, 1H), 3.25-3.36 (m, 1H), 2.99-3.13 (m, 1H), 2.62 (s, 3H) |
| 157 | 300 MHz, | 8.79 (s, 1H), 8.13 (dd, J = 4.1, 1.9 Hz, 1H), 7.77 (d, J = 8.3 Hz, 2H), |
| | DMSO-d₆ | 7.44-7.59 (m, 2H), 7.39 (d, J = 8.2 Hz, 2H), 7.16-7.34 (m, 3H), |
| | | 5.25 (d, J = 4.4 Hz, 1H), 4.76 (dd, J = 6.4, 4.5 Hz, 1H), 4.34 (ddd, J = 11.3, |
| | | 7.9, 2.9 Hz, 1H), 4.08-4.21 (m, 1H), 3.17-3.28 (m, 1H), 2.91 (ddd, J = 14.2, |
| | | 7.7, 2.7 Hz, 1H), 1.31 (d, J = 6.4 Hz, 3H) |
| 158 | 300 MHz, | 8.80 (s, 1H), 8.13 (dd, J = 4.0, 2.0 Hz, 1H), 7.79 (d, J = 8.3 Hz, 2H), |
| | DMSO-d₆ | 7.41-7.59 (m, 2H), 7.37 (d, J = 8.3 Hz, 2H), 7.17-7.32 (m, 3H), |
| | | 5.28 (t, J = 5.8 Hz, 1H), 4.54 (d, J = 5.7 Hz, 2H), 4.26-4.41 (m, 1H), |
| | | 4.09-4.20 (m, 1H), 3.20-3.27 (m, 1H), 2.83-2.98 (m, 1H) |
| 159 | 300 MHz, | 1H NMR (CDCl₃) d: 8.51 (s, 1H), 8.30 (dd, J = 4.1, 1.9 Hz, 1H), |
| | CDCl₃ | 7.79 (dd, J = 8.4, 1.8 Hz, 2H), 7.51 (d, J = 6.9 Hz, 2H), 7.27-7.40 (m, 3H), |
| | | 7.17-7.25 (m, 2H), 4.94-5.13 (m, 1H), 4.30-4.51 (m, 1H), |
| | | 3.94-4.15 (m, 1H), 3.67-3.89 (m, 1H), 3.02-3.10 (m, 1H), 2.70-2.91 (m, 1H) |
| 160 | 300 MHz, | 8.24 (dd, J = 4.6, 1.5 Hz, 1H), 7.69-7.90 (m, 4H), 7.36-7.57 (m, 4H), |
| | MeOH-d₄ | 7.23 (d, J = 8.8 Hz, 1H), 4.41-4.53 (m, 1H), 4.12-4.26 (m, 1H), |
| | | 3.19 (dd, J = 9.1, 3.6 Hz, 1H), 2.90-3.03 (m, 1H) |
| 161 | 300 MHz, | 9.37 (s, 1H), 9.18 (s, 1H), 8.39-8.58 (m, 1H), 8.10-8.36 (m, 2H), |
| | DMSO-d₆ | 7.19-7.41 (m, 4H), 5.55 (d, J = 10.76 Hz, 1H), 5.15 (d, J = 10.76 Hz, 1H). |
| 162 | 300 MHz, | 8.56 (s, 1H), 8.29 (t, J = 2.9 Hz, 1H), 7.83 (s, 1H), 7.46-7.62 (m, 2H), |
| | DMSO-d₆ | 7.39 (d, J = 2.8 Hz, 2H), 7.25 (d, J = 8.6 Hz, 1H), 4.27-4.46 (m, 3H), |
| | | 4.07 (t, J = 9.9 Hz, 1H), 2.86-3.03 (m, 1H), 1.31 (t, J = 7.1 Hz, 3H). |
| 163 | 300 MHz, | 9.00 (s, 1H), 8.59 (br. s., 1H), 8.12 (dd, J = 3.9, 1.8 Hz, 1H), 8.08 (s, |
| | DMSO-d₆ | 1H), 7.41-7.60 (m, 2H), 7.23-7.35 (m, 2H), 7.20 (d, J = 9.8 Hz, 1H), |
| | | 4.24-4.42 (m, 1H), 4.01-4.19 (m, 2H), 3.24 (dd, J = 14.5, 7.7 Hz, 1H), |
| | | 2.68-2.90 (m, 1H) |
| 164 | 400 MHz, | 11.96 (br. s., 1H), 8.71 (s, 1H), 8.13-8.23 (m, 2H), 8.06 (br. s., 1H), |
| | DMSO-d₆ | 7.79 (dd, J = 2.74, 9.59 Hz, 1H), 7.57 (dd, J = 2.15, 12.32 Hz, 1H), |
| | | 7.49 (t, J = 8.71 Hz, 1H), 7.19-7.28 (m, 1H), 6.22-6.39 (m, 1H), |
| | | 4.37-4.53 (m, 1H), 4.20-4.33 (m, 1H), 3.25 (dd, J = 4.69, 7.04 Hz, 1H), 2.78 (ddd, |
| | | J = 3.03, 8.75, 14.23 Hz, 1H). |
| 165 | 400 MHz, | 11.96 (br. s., 1H), 9.03 (s, 1H), 8.89 (dd, J = 1.37, 4.70 Hz, 1H), |
| | DMSO-d₆ | 8.03-8.26 (m, 2H), 7.78 (dd, J = 2.54, 9.78 Hz, 1H), 7.65 (dd, J = 2.15, 12.13 Hz, |
| | | 1H), 7.44-7.62 (m, 2H), 7.31 (d, J = 8.61 Hz, 1H), 6.30 (d, J = 9.59 Hz, |
| | | 1H), 3.52 (s, 2H). |
| 166 | 400 MHz, | 9.47 (s, 1H), 8.73 (s, 1H), 8.30 (dd, J = 2.54, 3.52 Hz, 1H), 8.02 (s, 1H), |
| | DMSO-d₆ | 7.46-7.63 (m, 2H), 7.32-7.43 (m, 2H), 7.24 (dd, J = 1.17, 8.80 Hz, |
| | | 1H), 4.39 (td, J = 4.11, 11.93 Hz, 1H), 3.92-4.12 (m, 1H), 3.49 (td, J = 2.35, |
| | | 12.32 Hz, 1H), 2.89 (ddd, J = 3.81, 10.91, 14.62 Hz, 1H), 2.44 (s, |
| | | 3H) |
| 167 | 400 MHz, | 8.19 (dd, J = 4.1, 2.0 Hz, 1H), 7.72 (d, J = 2.0 Hz, 1H), 7.62 (d, J = 8.2 Hz, |
| | MeOH-d₄ | 1H), 7.49 (dd, J = 8.2, 2.2 Hz, 1H), 7.28-7.45 (m, 4H), |
| | | 7.18-7.26 (m, 1H), 4.33-4.45 (m, 1H), 4.03-4.14 (m, 1H), 3.23-3.29 (m, 1H), |
| | | 2.97-3.08 (m, 1H), 2.43 (s, 3H) |
| 168 | 400 MHz, | 8.16-8.28 (m, 2H), 8.04-8.11 (m, 1H), 7.89-8.03 (m, 2H), |
| | MeOH-d₄ | 7.36-7.57 (m, 4H), 7.22 (dd, J = 8.7, 1.1 Hz, 1H), 4.60 (s, 1H), 4.42-4.53 (m, 1H), |
| | | 4.39 (s, 1H), 4.08-4.24 (m, 1H), 3.16-3.29 (m, 1H), 2.96 (ddt, J = 14.9, |
| | | 11.7, 3.2 Hz, 1H), 1.53 (s, 3H), 1.47 (s, 3H) |
| 169 | 400 MHz, | 10.02 (s, 1H), 8.28-8.45 (m, 2H), 8.06 (d, J = 8.02 Hz, 1H), |
| | CDCl₃ | 7.32-7.39 (m, 1H), 7.27-7.32 (m, 2H), 7.17-7.24 (m, 2H), 4.33-4.45 (m, 3H), |
| | | 4.05 (dt, J = 2.15, 11.83 Hz, 1H), 3.52-3.61 (m, 1H), 2.94-3.10 (m, |
| | | 1H), 2.84 (s, 3H), 1.41 (t, J = 7.14 Hz, 3H) |
| 170 | 300 MHz, | 11.95 (br. s., 1H), 8.70 (s, 1H), 8.01-8.17 (m, 2H), 7.80 (dd, J = 9.6, 2.8 Hz, |
| | DMSO-d₆ | 1H), 7.68 (t, J = 8.1 Hz, 1H), 7.52 (d, J = 12.8 Hz, 1H), 7.32 (s, 1H), |
| | | 7.30 (dd, J = 8.5, 1.2 Hz, 1H), 7.25 (dd, J = 7.9, 4.3 Hz, 1H), 6.31 (d, J = 9.6 Hz, |
| | | 1H), 4.30 (ddd, J = 11.6, 9.2, 2.4 Hz, 1H), 4.18 (ddd, J = 11.3, |
| | | 6.4, 3.1 HZ, 1H), 3.23 (ddd, J = 14.5, 6.3, 2.4 HZ, 1H), 2.81 (ddd, J = 14.0, |
| | | 9.2, 3.1 Hz, 1H) |
| 171 | 300 MHz, | 8.95 (dd, J = 2.0, 0.8 Hz, 1H), 8.35 (dd, J = 8.2, 2.0 Hz, 1H), |
| | MeOH-d₄ | 8.24-8.28 (m, 1H), 8.20-8.24 (m, 1H), 7.45 (dd, J = 11.9, 2.3 Hz, 1H), |
| | | 7.32-7.42 (m, 3H), 7.20-7.27 (m, 1H), 4.41 (dt, J = 11.9, 4.0 Hz, 1H), 4.05 (td, J = 11.7, |
| | | 2.4 Hz, 1H), 3.34-3.45 (m, 1H), 2.98-3.13 (m, 1H) |
| 172 | 400 MHz, | 9.59 (s, 1 H), 9.11 (d, J = 1.56 Hz, 1 H), 8.49 (dd, J = 8.22, 2.15 Hz, 1 |
| | DMSO-d₆ | H), 8.31 (t, J = 2.93 Hz, 1 H), 8.17 (d, J = 8.02 Hz, 1 H), 7.75 (t, J = 8.12 Hz |
| | | 1 H), 7.60 (d, J = 12.72 Hz, 1 H), 7.36-7.45 (m, 3 H), |
| | | 4.36-4.47 (m, 1 H), 4.06-4.15 (m, 1 H), 3.91 (s, 3 H), 3.44-3.54 (m, 1 H), |
| | | 2.95 (ddd, J = 14.43, 10.51, 3.62 Hz, 1 H). |
| 173 | 400 MHz, | 8.09 (dd, J = 4.30, 1.57 Hz, 1 H), 8.05-8.07 (m, 1 H), 7.95 (dd, J = 9.59, |
| | MeOH-d₄ | 2.54 Hz, 1 H), 7.26-7.36 (m, 2 H), 7.05-7.16 (m, 3 H), 6.50 (d, J = 9.59 Hz, |
| | | 1 H), 4.28-4.37 (m, 1 H), 4.10 (ddd, J = 11.44, 8.22, 2.84 Hz, |
| | | 1 H), 3.56 (ddd, J = 14.43, 8.07, 2.93 Hz, 1 H), 2.68 (ddd, J = 14.43, |
| | | 7.48, 2.74 Hz, 1 H). |
| 174 | 400 MHz, | 9.10 (d, J = 1.37 Hz, 1 H), 8.91 (s, 1 H), 8.51 (dd, J = 8.12, 2.05 Hz, 1 |
| | DMSO-d₆ | H), 8.20 (d, J = 8.22 Hz, 1 H), 8.14 (dd, J = 4.30, 1.57 Hz, 1 H), |
| | | 7.29-7.41 (m, 4 H), 7.22 (d, J = 8.80 Hz, 1 H), 4.19-4.31 (m, 2 H), 3.91 (s, 3 |
| | | H), 3.48-3.59 (m, 1 H), 2.70-2.81 (m, 1 H). |
| 175 | 300 MHz, | 9.62 (s, 1H), 9.08-9.14 (m, 1H), 8.50 (dd, J = 8.2, 2.0 Hz, 1H), 8.31 (t, |
| | DMSO-d₆ | J = 3.0 Hz, 1H), 8.14-8.22 (m, 1H), 7.66-7.76 (m, J = 8.5 Hz, 2H), |
| | | 7.57-7.66 (m, J = 8.3 Hz, 2H), 7.40 (d, J = 2.9 Hz, 2H), 4.42 (dt, J = 11.8, 4.1 Hz, |
| | | 1H), 4.03 (dd, J = 11.3, 9.1 Hz, 1H), 3.92 (s, 3H), 3.41-3.53 (m, 1H), |
| | | 2.99 (ddd, J = 14.6, 11.0, 3.8 Hz, 1H) |
| 176 | 300 MHz, | 9.63 (s, 1H), 9.11 (dd, J = 2.0, 0.6 Hz, 1H), 8.49 (dd, J = 8.2, 2.0 Hz, |
| | DMSO-d₆ | 1H), 8.31 (t, J = 3.0 Hz, 1H), 8.18 (dd, J = 8.2, 0.6 Hz, 1H), |
| | | 7.45-7.57 (m, 2H), 7.28-7.43 (m, 4H), 4.41 (dt, J = 11.7, 3.9 Hz, 1H), |
| | | 3.94-4.07 (m, 1H), 3.92 (s, 3H), 3.38-3.53 (m, 1H), 2.96 (ddd, J = 14.6, 11.2, 3.9 Hz, |
| | | 1H). |
| 177 | 300 MHz, | 9.32 (s, 2H), 8.32 (dd, J = 4.5, 1.6 Hz, 1H), 7.38-7.63 (m, 4H), |
| | MeOH-d₄ | 7.22-7.31 (m, 1H), 4.43-4.59 (m, 1H), 4.09-4.29 (m, 1H), 3.20-3.30 (m, |
| | | 1H), 3.11 (dd, J = 10.2, 3.8 Hz, 1H) |
| 178 | 300 MHz, | 8.45-8.52 (m, 1H), 8.27 (dd, J = 4.8, 1.3 Hz, 1H), 8.20 (d, J = 7.9 Hz, |
| | MeOH-d₄ | 1H), 8.02-8.10 (m, 1H), 7.51-7.70 (m, 3H), 7.35-7.50 (m, 2H), |
| | | 7.20-7.26 (m, 1H), 4.46-4.59 (m, 1H), 4.25 (ddd, J = 11.7, 8.4, 2.9 Hz, 1H), |
| | | 3.18 (ddd, J = 14.6, 8.4, 3.3 Hz, 1H), 2.89 (ddd, J = 14.7, 7.2, 3.0 Hz, |
| | | 1H). |
| 179 | 300 MHz, | 8.65 (s, 1H), 8.24-8.34 (m, 1H), 7.66 (s, 2H), 7.45-7.59 (m, 2H), |
| | DMSO-d₆ | 7.33-7.42 (m, 2H), 7.21 (d, J = 8.6 Hz, 1H), 4.37 (dt, J = 12.0, 4.1 Hz, 1H), |
| | | 3.95-4.11 (m, 1H), 3.68 (s, 3H), 3.45 (d, J = 16.7 Hz, 1H), |
| | | 2.79-2.94 (m, 1H) |
| 180 | 300 MHz, | 8.78 (s, 1H), 8.12 (dd, J = 4.0, 1.8 Hz, 1H), 7.72 (s, 1H), 7.42-7.57 (m, |
| | DMSO-d₆ | 2H), 7.15-7.35 (m, 3H), 4.33 (ddd, J = 11.2, 8.0, 2.7 Hz, 1H), |
| | | 4.05-4.18 (m, 1H), 3.70 (s, 3H), 3.23 (ddd, J = 14.3, 7.4, 2.6 Hz, 1H), |
| | | 2.80 (ddd, J = 14.2, 7.9, 2.8 Hz, 1H) |
| 181 | 300 MHz, | 8.44 (s, 1H), 8.32 (dd, J = 4.1, 1.9 Hz, 1H), 7.45 (s, 2H), 7.28-7.40 (m, |
| | CDCl₃ | 3H), 7.20-7.26 (m, 2H), 4.43 (dt, J = 11.8, 3.7 Hz, 1H), 4.04 (td, J = 12.3, |
| | | 2.0 Hz, 1H), 3.92 (s, 3H), 3.72-3.86 (m, 1H), 2.82 (ddd, J = 14.7, |
| | | 12.8, 4.2 Hz, 1H), 2.34 (s, 6H) |
| 182 | 400 MHz, | 8.90 (s, 1H), 8.10-8.17 (m, 2H), 8.02 (dd, J = 8.8, 2.2 Hz, 1H), |
| | DMSO-d₆ | 7.43-7.56 (m, 2H), 7.24-7.34 (m, 2H), 7.16-7.24 (m, 2H), 4.32 (t, J = 8.3 Hz, |
| | | 1H), 4.08-4.18 (m, 1H), 3.87 (s, 3H), 3.79 (s, 3H), 3.20-3.34 (m, |
| | | 2H), 2.76-2.89 (m, 1H) |
| 183 | 300 MHz, | 9.19 (d, J = 1.4 Hz, 1H), 8.53 (dd, J = 8.2, 2.2 Hz, 1H), 8.26-8.33 (m, |
| | MeOH-d₄ | 1H), 8.21 (d, J = 8.0 Hz, 1H), 7.60 (d, J = 2.3 Hz, 1H), 7.52 (d, J = 8.4 Hz, |
| | | 1H), 7.39 (d, J = 3.1 Hz, 2H), 7.34 (dd, J = 8.6, 2.3 Hz, 1H), |
| | | 4.44 (dt, J = 11.7, 4.0 Hz, 1H), 4.07 (td, J = 11.6, 2.2 Hz, 1H), 4.00 (s, 3H), |
| | | 3.36-3.43 (m, 1H), 3.05-3.21 (m, 1H) |
| 184 | 300 MHz, | 8.21 (dd, J = 4.1, 2.0 Hz, 1H), 7.91-8.04 (m, 4H), 7.30-7.51 (m, 4H), |
| | MeOH-d₄ | 7.21-7.28 (m, 1H), 4.31-4.51 (m, 1H), 3.95-4.23 (m, 1H), |
| | | 2.95-3.15 (m, 1H) |
| 185 | 300 MHz, | 8.25 (dd, J = 4.7, 1.4 Hz, 1H), 8.11 (d, J = 2.3 Hz, 1H), 7.97 (dd, J = 9.7, |
| | MeOH-d₄ | 2.6 Hz, 1H), 7.61-7.75 (m, 2H), 7.54-7.59 (m, 1H), 7.46-7.52 (m, |
| | | 1H), 7.36 (t, J = 9.4 Hz, 1H), 6.54 (d, J = 9.8 Hz, 1H), 4.33-4.66 (m, |
| | | 1H), 4.17 (ddd, J = 11.7, 9.0, 2.7 Hz, 1H), 3.25 (ddd, J = 14.5, 9.1, 3.2 Hz, |
| | | 1H), 2.89 (ddd, J = 14.6, 6.7, 2.7 Hz, 1H) |
| 186 | 400 MHz, | 13.15 (br s., 1H), 9.40 (s, 1H), 8.12-8.26 (m, 1H), 7.80 (t, J = 6.1 Hz, |
| | DMSO-d₆ | 2H), 7.64 (d, J = 10.8 Hz, 1H), 7.52 (t, J = 8.3 Hz, 1H), 7.29-7.39 (m, |
| | | 3H), 7.26 (t, J = 7.6 Hz, 1H), 4.35-4.49 (m, 1H), 4.18 (t, J = 8.8 Hz, |
| | | 1H), 3.79 (s, 3H), 3.16-3.25 (m, 1H), 2.99-3.14 (m, 1H). |
| 187 | 400 MHz, | 12.22 (br. s., 1H), 8.38 (s, 1H), 8.09 (dd, J = 4.3, 1.8 Hz, 1H), 7.54 (s, |
| | DMSO-d₆ | 1H), 7.41-7.51 (m, 2H), 7.15-7.36 (m, 4H), 4.26-4.33 (m, 1H), |
| | | 4.23 (q, J = 7.2 Hz, 2H), 4.11-4.18 (m, 1H), 3.26 (dd, J = 7.0, 4.5 Hz, 1H), |
| | | 2.79 (ddd, J = 14.1, 8.7, 2.8 Hz, 1H), 1.26 (t, J = 7.0 Hz, 3H). |
| 188 | 400 MHz, | 8.83 (s, 1H), 8.13 (dd, J = 4.1, 2.0 Hz, 1H), 7.72 (d, J = 8.2 Hz, 1H), |
| | DMSO-d₆ | 7.42-7.57 (m, 2H), 7.20-7.33 (m, 3H), 7.16 (d, J = 1.6 Hz, 1H), |
| | | 6.88 (dd, J = 8.3, 1.7 Hz, 1H), 6.75 (s, 2H), 4.34 (ddd, J = 11.2, 7.8, 3.0 Hz, |
| | | 1H), 4.13 (ddd, J = 11.2, 8.0, 2.9 Hz, 1H), 3.80 (s, 3H), 3.19 (ddd, J = 14.3, |
| | | 7.8, 2.9 Hz, 1H), 2.92 (ddd, J = 14.3, 7.6, 2.7 Hz, 1H). |
| 189 | 300 MHz, | 9.19 (s, 1H), 8.35 (dd, J = 4.2, 1.7 Hz, 1H), 7.78 (s, 1H), 7.27-7.37 (m, |
| | CDCl₃ | 2H), 7.16-7.24 (m, 3H), 4.41 (dt, J = 11.8, 3.7 Hz, 1H), 4.01 (td, J = 12.3, |
| | | 2.1 Hz, 1H), 3.72 (dt, J = 14.8, 2.6 Hz, 1H), 2.81 (ddd, J = 14.5, |
| | | 12.7, 4.2 Hz, 1H) |
| 190 | 300 MHz, | 9.08 (s, 1H), 8.81 (s, 1H), 8.42 (s, 1H), 8.30 (dd, J = 4.2, 1.7 Hz, 1H), |
| | CDCl₃ | 7.27-7.39 (m, 3H), 7.22 (d, J = 2.2 Hz, 2H), 4.35-4.55 (m, 1H), |
| | | 3.93-4.08 (m, 1H), 3.80 (dt, J = 14.7, 2.4 Hz, 1H), 2.76 (ddd, J = 14.5, 13.2, |
| | | 4.3 Hz, 1H) |
| 191 | 400 MHz, | 9.01 (s, 1H), 8.13 (dd, J = 4.2, 1.7 Hz, 1H), 7.75-7.83 (m, 2H), |
| | DMSO-d₆ | 7.66-7.75 (m, 1H), 7.45-7.57 (m, 2H), 7.17-7.33 (m, 3H), 4.34 (ddd, J = 11.2, 7.9, |
| | | 2.8 Hz, 1H), 4.12 (ddd, J = 11.2, 7.9, 2.7 Hz, 1H), 3.85 (s, 3H), |
| | | 3.22-3.30 (m, 1H), 2.90 (q, J = 7.6 Hz, 2H), 2.77-2.87 (m, 1H), 1.16 (t, J = 7.4 Hz, |
| | | 3H). |
| 192 | 400 MHz, | 9.38 (s, 1H), 8.46-8.63 (m, 3H), 8.14 (dd, J = 4.2, 1.7 Hz, 1H), 7.56 (dd, |
| | DMSO-d₆ | J = 12.3, 2.2 Hz, 1H), 7.50 (t, J = 8.0 Hz, 1H), 7.21-7.34 (m, 3H), |
| | | 4.38 (q, J = 7.0 Hz, 4H), 4.29-4.35 (m, 1H), 4.09-4.17 (m, 1H), 3.32-3.36 (m, |
| | | 1H), 2.85 (ddd, J = 14.2, 8.0, 2.8 Hz, 1H), 1.35 (t, J = 7.1 Hz, 6H). |
| 193 | 400 MHz, | 8.74 (s, 1H), 8.11 (dd, J = 4.1, 1.8 Hz, 1H), 7.42-7.52 (m, 2H), 7.40 (t, J = 1.9 Hz, |
| | DMSO-d₆ | 1H), 7.22-7.36 (m, 2H), 7.10-7.20 (m, 1H), 6.76 (t, J = 2.4 Hz, |
| | | 1H), 6.37 (dd, J = 2.7, 1.8 Hz, 1H), 4.73 (q, J = 16.0 Hz, 2H), 4.28 (ddd, |
| | | J = 11.4, 8.5, 2.6 Hz, 1H), 4.08-4.17 (m, 1H), 3.68 (s, 3H), 3.08-3.16 (m, |
| | | 1H), 2.74 (ddd, J = 14.4, 8.3, 2.9 Hz, 1H). |
| 194 | 300 MHz, | 8.30 (dd, J = 5.0, 1.3 Hz, 1H), 8.15 (d, J = 2.5 Hz, 1H), 7.99 (dd, J = 9.6, |
| | MeOH-d₄ | 2.7 Hz, 1H), 7.76 (dd, J = 8.4, 1.2 Hz, 1H), 7.65 (dd, J = 8.6, 5.1 Hz, |
| | | 1H), 7.57 (d, J = 8.6 Hz, 1H), 7.53 (d, J = 2.2 Hz, 1H), 7.22 (dd, J = 8.6, |
| | | 2.3 Hz, 1H), 6.54 (d, J = 9.6 Hz, 1H), 4.45-4.62 (m, 1H), 4.23 (ddd, J = 11.6, |
| | | 8.5, 2.9 Hz, 1H), 3.16 (ddd, J = 14.5, 8.4, 3.1 Hz, 1H), |
| | | 2.59-2.89 (m, 1H) |
| 195 | 300 MHz, | 8.29 (dd, J = 4.8, 1.3 Hz, 1H), 7.96 (q, J = 8.5 Hz, 4H), 7.63-7.71 (m, |
| | MeOH-d₄ | 1H), 7.53-7.61 (m, 1H), 7.38-7.51 (m, 2H), 7.25 (d, J = 8.8 Hz, 1H), |
| | | 4.41-4.67 (m, 1H), 4.13-4.37 (m, 1H), 3.13-3.27 (m, 1H), |
| | | 2.81-3.01 (m, 1H) |
| 196 | 300 MHz, | 8.59 (s, 1H), 8.33 (dd, J = 4.1, 1.8 Hz, 1H), 8.02 (s, 1H), 7.69 (d, J = 8.4 Hz, |
| | CDCl₃ | 1H), 7.47 (dd, J = 8.3, 1.3 Hz, 1H), 7.42 (dd, J = 11.7, 2.2 Hz, 1H), |
| | | 7.26-7.33 (m, 4H), 7.23 (d, J = 8.6 Hz, 1H), 4.40-4.49 (m, 1H), |
| | | 4.12 (s, 3H), 4.07 (td, J = 12.2, 2.2 Hz, 1H), 3.92 (s, 3H), 3.84 (dt, J = 14.9, |
| | | 2.5 Hz, 1H), 2.80-2.94 (m, 1H). |
| 197 | 300 MHz, | 9.22 (s, 1H), 8.35 (dd, J = 5.0, 1.4 Hz, 1H), 7.89-7.99 (m, 2H), |
| | CDCl₃ | 7.79 (dd, J = 8.2, 1.6 Hz, 1H), 7.71 (dd, J = 8.5, 1.3 Hz, 1H), 7.60 (dd, J = 8.5, |
| | | 5.1 Hz, 1H), 7.34 (t, J = 7.7 Hz, 1H), 7.11-7.17 (m, 1H), 7.04 (d, J = 8.6 Hz, |
| | | 1H), 4.45-4.57 (m, 1H), 4.01 (t, J = 11.6 Hz, 1H), |
| | | 3.52-3.69 (m, 1H), 2.67-2.74 (m, 1H) |
| 198 | 300 MHz, | 9.38 (s, 1H), 8.76 (d, J = 2.2 Hz, 1H), 8.35 (dd, J = 3.9, 2.1 Hz, 1H), |
| | CDCl₃ | 8.15 (d, J = 2.0 Hz, 1H), 7.31-7.42 (m, 1H), 7.15-7.31 (m, 4H), |
| | | 4.37-4.44 (m, 1H), 3.97-4.13 (m, 1H), 3.60-3.72 (m, 1H), 2.89 (m, 1H) |
| 199 | 300 MHz, | 11.80 (br. s., 1H), 8.29 (dd, J = 3.6, 2.4 Hz, 2H), 8.00 (br. s., 1H), |
| | CDCl₃ | 7.82 (dd, J = 9.6, 2.6 Hz, 1H), 7.30 (br. s., 2H), 7.23 (s, 1H), 7.04-7.20 (m, |
| | | 2H), 6.55 (d, J = 9.6 Hz, 1H), 4.29-4.46 (m, 1H), 3.89-4.07 (m, 1H), |
| | | 3.58 (br. s., 1H), 2.84 (br. s., 1H), 2.24 (s, 3H) |
| 200 | 300 MHz, | 8.90 (s, 1H), 8.32 (dd, J = 4.2, 1.8 Hz, 1H), 7.26-7.38 (m, 3H), |
| | CDCl₃ | 7.16-7.24 (m, 2H), 6.59 (s, 1H), 4.41 (dt, J = 11.7, 3.7 Hz, 1H), 4.01 (td, J = 12.3, |
| | | 2.2 Hz, 1H), 3.66 (dt, J = 14.8, 2.6 Hz, 1H), 2.80 (ddd, J = 14.6, |
| | | 12.7, 4.2 Hz, 1H) |
| 201 | 300 MHz, | 9.90 (s, 1H), 8.64-8.77 (m, 2H), 8.36 (dd, J = 3.8, 2.0 Hz, 1H), |
| | CDCl₃ | 7.98 (dd, J = 5.0, 1.6 Hz, 1H), 7.32-7.41 (m, 1H), 7.15-7.32 (m, 4H), |
| | | 4.41 (dt, J = 11.7, 3.9 Hz, 1H), 4.01-4.11 (m, 1H), 3.97 (s, 3H), |
| | | 3.59-3.70 (m, 1H), 2.99 (ddd, J = 14.7, 12.0, 4.0 Hz, 1H) |
| 202 | 300 MHz, | 8.25 (d, J = 2.5 Hz, 1H), 8.17 (dd, J = 3.9, 2.0 Hz, 1H), 7.86 (dd, J = 9.5, |
| | MeOH-d₄ | 2.6 Hz, 1H), 7.25-7.46 (m, 4H), 7.12-7.23 (m, 1H), 6.52 (d, J = 9.5 Hz, |
| | | 1H), 4.31-4.44 (m, 1H), 4.26 (dd, J = 13.2, 2.9 Hz, 1H), |
| | | 3.99-4.16 (m, 2H), 3.64 (dd, J = 13.1, 8.7 Hz, 1H), 3.34-3.43 (m, 1H), |
| | | 2.82-2.98 (m, 1H), 1.22 (d, J = 6.3 Hz, 3H) |
| 203 | 300 MHz, | 8.94 (s, 1H), 8.12 (dd, J = 4.1, 1.9 Hz, 1H), 7.79 (d, J = 4.1 Hz, 1H), |
| | DMSO-d₆ | 7.42-7.56 (m, 2H), 7.22-7.35 (m, 3H), 7.13-7.22 (m, 1H), |
| | | 4.23-4.39 (m, 1H), 4.05-4.16 (m, 1H), 3.17-3.28 (m, 1H), 2.77 (ddd, J = 14.2, |
| | | 7.6, 2.8 Hz, 1H) |
| 204 | 300 MHz, | 8.20 (dd, J = 3.8, 2.2 Hz, 1H), 8.01-8.09 (m, 2H), 7.85-7.95 (m, 2H), |
| | MeOH-d₄ | 7.27-7.50 (m, 4H), 7.17-7.27 (m, 1H), 4.35-4.47 (m, 1H), |
| | | 4.04-4.14 (m, 1H), 3.25-3.36 (m, 1H), 2.99-3.13 (m, 1H), 2.62 (s, 3H) |
| 205 | 300 MHz, | 8.22-8.30 (m, 1H), 7.66-7.76 (m, 4H), 7.47-7.63 (m, 5H), |
| | MeOH-d₄ | 4.46-4.55 (m, 1H), 4.17-4.27 (m, 1H), 3.09-3.27 (m, 1H), 2.75-2.88 (m, 1H) |
| 206 | 300 MHz, | 9.48 (s, 1H), 8.80 (dd, J = 2.2, 0.6 Hz, 1H), 8.23-8.35 (m, 2H), |
| | DMSO-d₆ | 7.92-8.03 (m, 1H), 7.47-7.64 (m, 2H), 7.33-7.44 (m, 2H), 7.26 (dt, J = 8.7, |
| | | 1.2 Hz, 1H), 4.40 (dt, J = 11.9, 4.2 Hz, 1H), 3.97-4.12 (m, 1H), |
| | | 3.42-3.58 (m, 1H), 2.90 (ddd, J = 14.7, 10.9, 3.8 Hz, 1H) |
| 207 | 300 MHz, | 8.15 (dd, J = 4.8, 1.4 Hz, 1H), 7.48-7.55 (m, 1H), 7.41-7.47 (m, 1H), |
| | MeOH-d₄ | 7.36-7.41 (m, 2H), 7.22-7.36 (m, 2H), 7.09 (dd, J = 8.7, 1.2 Hz, 1H), |
| | | 4.32-4.44 (m, 1H), 4.10 (ddd, J = 11.7, 8.7, 2.9 Hz, 1H), 3.06 (ddd, J = 14.5, |
| | | 8.6, 3.3 Hz, 1H), 2.74 (ddd, J = 14.5, 6.9, 2.9 Hz, 1H). |
| 208 | 300 MHz, | 8.23 (t, J = 3.0 Hz, 1H), 7.29-7.48 (m, 4H), 7.17-7.26 (m, 1H), |
| | MeOH-d₄ | 7.11 (d, J = 3.7 Hz, 1H), 6.59 (d, J = 3.5 Hz, 1H), 4.39 (dt, J = 11.9, 4.3 Hz, |
| | | 1H), 3.98-4.09 (m, 1H), 3.04-3.25 (m, 2H) |
| 209 | 300 MHz, | 8.23 (t, J = 3.0 Hz, 1H), 7.29-7.48 (m, 4H), 7.17-7.26 (m, 1H), |
| | MeOH-d₄ | 7.11 (d, J = 3.7 Hz, 1H), 6.59 (d, J = 3.5 Hz, 1H), 4.39 (dt, J = 11.9, 4.3 Hz, |
| | | 1H), 3.98-4.09 (m, 1H), 3.04-3.25 (m, 2H) |
| 210 | 400 MHz | 9.34 (s, 1H), 8.41 (s, 1H), 7.43-7.40 (m, 2H), 7.35-7.30 (m, 2H), |
| | CDCl₃ | 7.21-7.15 (m, 5H), 4.47 (s, 1H), 4.10 (s, 1H), 3.10-2.90 (s, 2H) |
| 211 | 400 MHz | 8.32-8.27 (m, 1H), 8.20 (s, 1H), 7.99 (s, 1H), 7.45-7.31 (m, 5H), |
| | CDCl₃ | 7.24-7.12 (m, 2H), 6.68 (s, 1H), 4.42 (d, J = 11.4 Hz, 1H), 4.05-3.96 (m, |
| | | 1H), 3.60-3.40 (m, 1H), 3.15-2.90 (m, 1H) |
| 212 | 400 MHz | 9.21 (s, 1H), 8.40 (s, 1H), 8.11 (s, 1H), 7.51-7.43 (m, 2H), |
| | CDCl₃ | 7.40-7.35 (m, 2H), 7.21-7.16 (m, 2H), 4.46 (d, J = 11.8 Hz, 1H), 4.02 (s, 1H), |
| | | 3.21 (s, 2H) |
| 213 | 400 MHz | 8.58 (s, 1H), 8.50 (d, J = 1.8 Hz, 1H), 8.32 (dd, J = 3.9, 2.1 Hz, 1H), |
| | CDCl₃ | 7.57 (d, J = 2.6 Hz, 1H), 7.47-7.40 (m, 2H), 7.39-7.31 (m, 2H), |
| | | 7.21-7.14 (m, 2H), 4.47-4.39 (m, 1H), 4.01 (ddd, J = 13.2, 11.8, 2.2 Hz, |
| | | 1H), 3.65 (s, 1H), 2.90 (s, 1H) |
| 214 | 400 MHz | 9.60 (s, 1H), 9.20 (s, 1H), 8.36 (dd, J = 3.4, 2.6 Hz, 1H), 7.51-7.46 (m, |
| | CDCl₃ | 2H), 7.34-7.29 (m, 2H), 7.18 (dq, J = 8.9, 1.0 Hz, 2H), 4.47-4.40 (m, |
| | | 1H), 4.06 (ddd, J = 12.7, 11.7, 2.2 Hz, 1H), 3.59 (d, J = 14.6 Hz, 1H), |
| | | 3.10-2.99 (m, 1H). |
| 215 | 400 MHz | 8.63 (s, 1H), 8.33 (dd, J = 3.4, 2.6 Hz, 1H), 8.12 (s, 2H), 7.81-7.76 (m, |
| | CDCl₃ | 1H), 7.57 (dd, J = 8.4, 1.3 Hz, 1H), 7.51-7.43 (m, 2H), 7.38-7.31 (m, |
| | | 2H), 7.19-7.12 (m, 2H), 4.45 (dt, J = 11.7, 3.6 Hz, 1H), 4.05 (ddd, J = 12.8, |
| | | 11.7, 2.1 Hz, 1H), 3.64 (s, 1H), 3.09-2.99 (m, 1H). |
| 216 | 400 MHz | 8.71 (s, 1H), 8.45-8.39 (m, 1H), 8.34 (d, J = 3.0 Hz, 1H), 8.15 (d, J = 7.9 Hz, |
| | CDCl₃ | 1H), 7.44 (d, J = 8.6 Hz, 2H), 7.41-7.35 (m, 2H), |
| | | 7.25-7.15 (m, 3H), 7.03 (s, 1H), 4.46 (d, J = 11.8 Hz, 1H), 4.04 (t, J = 12.0 Hz, |
| | | 1H), 3.58 (s, 1H), 3.07-2.95 (m, 1H). |
| 217 | 400 MHz | 9.26 (s, 1H), 8.40-8.37 (m, 1H), 8.18-8.12 (m, 2H), 7.68-7.60 (m, |
| | CDCl₃ | 1H), 7.53-7.46 (m, 2H), 7.32-7.22 (m, 3H), 7.17-7.11 (m, 2H), |
| | | 6.88 (t, J = 6.9 Hz, 1H), 4.42 (dt, J = 11.7, 3.9 Hz, 1H), 4.08 (td, J = 11.7, 2.3 Hz, |
| | | 1H), 3.48 (s, 1H), 3.14 (s, 1H). |
| 218 | 400 MHz | 9.72 (s, 1H), 8.79 (s, 1H), 8.32 (dd, J = 3.9, 2.0 Hz, 2H), 7.55-7.51 (m, |
| | CDCl₃ | 3H), 7.48 (d, J = 3.7 Hz, 1H), 7.31-7.28 (m, 2H), 7.16 (ddt, J = 7.7, |
| | | 2.0, 1.0 Hz, 2H), 6.93 (d, J = 3.6 Hz, 1H), 4.45 (ddd, J = 11.7, 4.2, 3.0 Hz, |
| | | 1H), 4.07 (ddd, J = 12.9, 11.7, 2.2 Hz, 1H), 3.87 (dt, J = 14.6, 2.5 Hz, |
| | | 1H), 2.92 (ddd, J = 14.5, 12.9, 4.2 Hz, 1H). |
| 219 | 400 MHz | 9.01 (s, 1H), 8.43 (dd, J = 7.0, 1.1 Hz, 1H), 8.40-8.36 (m, 1H), |
| | CDCl₃ | 7.58 (dt, J = 8.8, 1.2 Hz, 1H), 7.49-7.44 (m, 2H), 7.34 (ddddq, J = 4.0, 3.5, |
| | | 2.0, 1.0, 0.5 Hz, 2H), 7.20-7.15 (m, 2H), 7.15-7.11 (m, 1H), 7.03 (d, |
| | | J = 0.9 Hz, 1H), 6.84 (td, J = 6.9, 1.4 Hz, 1H), 4.43 (d, J = 11.6 Hz, 1H), |
| | | 4.06 (t, J = 11.7 Hz, 1H), 3.40-3.48 (m, 1H), 3.13-3.23 (m, 1H). |
| 220 | 400 MHz | 8.67 (s, 1H), 8.32 (dd, J = 3.5, 2.5 Hz, 1H), 7.46-7.40 (m, 2H), |
| | CDCl₃ | 7.28-7.27 (m, 1H), 7.27 (dq, J = 1.5, 0.5 Hz, 1H), 7.13 (ddt, J = 7.8, 1.9, 1.0 Hz, |
| | | 2H), 5.99 (s, 1H), 4.38 (dt, J = 11.5, 3.8 Hz, 1H), 4.31-4.24 (m, |
| | | 2H), 4.17 (t, J = 6.3 Hz, 2H), 4.03 (td, J = 11.8, 2.3 Hz, 1H), |
| | | 3.49-3.38 (m, 1H), 3.14-3.01 (m, 1H), 2.29-2.22 (m, 2H). |
| 221 | 400 MHz | 12.98 (s, 1H), 8.86 (dd, J = 4.2, 1.8 Hz, 1H), 8.80 (dd, J = 7.5, 1.6 Hz, |
| | CDCl₃ | 1H), 8.38 (d, J = 4.4 Hz, 1H), 8.27 (dd, J = 8.3, 1.8 Hz, 1H), 7.97 (dd, J = 8.1, |
| | | 1.5 Hz, 1H), 7.67 (dd, J = 8.1, 7.4 Hz, 1H), 7.57-7.51 (m, 2H), |
| | | 7.47 (dd, J = 8.3, 4.2 Hz, 1H), 7.37-7.27 (m, 2H), 7.23-7.17 (m, 2H), |
| | | 4.41 (dt, J = 11.6, 4.1 Hz, 1H), 4.13 (td, J = 11.4, 2.3 Hz, 1H), 3.51 (d, J = 8.0 Hz, |
| | | 1H), 3.11 (s, 1H). |
| 222 | 400 MHz | 8.33 (s, 1H), 7.48 (d, J = 9.5 Hz, 1H), 7.43-7.31 (m, 4H), |
| | CDCl₃ | 7.23-7.15 (m, 3H), 7.14-7.10 (m, 1H), 6.96 (s, 1H), 4.51 (s, 1H), 4.08 (t, J = 12.0 Hz, |
| | | 1H), 2.63 (s, 2H), 2.43 (d, J = 0.8 Hz, 3H) |
| 223 | 400 MHz | 9.95 (s, 1H), 8.70 (s, 1H), 8.39 (d, J = 8.4 Hz, 1H), 8.33 (dd, J = 3.4, 2.6 Hz, |
| | CDCl₃ | 1H), 7.98 (s, 1H), 7.82 (dd, J = 8.4, 1.7 Hz, 1H), 7.51-7.43 (m, |
| | | 2H), 7.40-7.30 (m, 2H), 7.18 (ddt, J = 7.9, 2.1, 1.0 Hz, 2H), 7.11 (d, J = 6.3 Hz, |
| | | 1H), 6.56 (d, J = 7.2 Hz, 1H), 4.44 (dt, J = 11.7, 3.6 Hz, 1H), |
| | | 4.03 (td, J = 12.5, 11.7, 2.1 Hz, 1H), 3.61 (s, 1H), 3.01 (d, J = 9.3 Hz, |
| | | 1H). |
| 224 | 400 MHz | 9.16 (s, 1H), 8.38 (d, J = 4.4 Hz, 1H), 8.33 (dd, J = 3.6, 2.3 Hz, 1H), |
| | CDCl₃ | 7.49 (d, J = 2.1 Hz, 1H), 7.47 (d, J = 2.1 Hz, 1H), 7.32-7.28 (m, 2H), |
| | | 7.22 (s, 1H), 7.17 (q, J = 1.1 Hz, 1H), 7.15 (q, J = 1.0 Hz, 1H), 6.39 (d, J = 4.4 Hz, |
| | | 1H), 4.42 (dt, J = 11.7, 3.8 Hz, 1H), 4.05 (td, J = 12.0, 2.2 Hz, |
| | | 1H), 3.55 (d, J = 14.0 Hz, 1H), 3.20-3.09 (m, 1H), 2.91 (tt, J = 8.5, 5.2 Hz, |
| | | 1H), 1.43-1.34 (m, 2H), 1.17-1.08 (m, 2H) |
| 225 | 400 MHz | 9.24 (d, J = 2.2 Hz, 1H), 8.75 (s, 1H), 8.62 (s, 1H), 8.34 (dd, J = 4.2, 1.7 Hz, |
| | CDCl₃ | 1H), 7.84 (d, J = 9.0 Hz, 1H), 7.58 (s, 1H), 7.54-7.48 (m, 2H), |
| | | 7.33-7.30 (m, 2H), 7.29 (t, J = 1.8 Hz, 1H), 7.17 (dq, J = 9.0, 1.0 Hz, 2H), |
| | | 4.45 (ddd, J = 11.7, 4.2, 2.8 Hz, 1H), 4.09-4.05 (m, 1H), 4.01 (s, 3H), |
| | | 3.84 (dt, J = 14.5, 2.5 Hz, 1H), 2.87 (td, J = 13.8, 4.2 Hz, 1H). |
| 226 | 400 MHz | 8.98 (s, 1H), 8.35 (dd, J = 3.9, 2.1 Hz, 1H), 8.21 (d, J = 1.0 Hz, 1H), |
| | CDCl₃ | 7.86 (d, J = 1.0 Hz, 1H), 7.49-7.41 (m, 2H), 7.27-7.26 (m, 2H), |
| | | 7.14 (ddt, J = 7.9, 2.1, 1.1 Hz, 2H), 4.39 (dt, J = 11.7, 3.7 Hz, 1H), 4.02 (ddd, |
| | | J = 12.5, 11.6, 2.2 Hz, 1H), 3.61 (dt, J = 14.9, 2.7 Hz, 1H), 2.95 (ddd, J = 14.5, |
| | | 12.6, 4.1 Hz, 1H) |
| 227 | 400 MHz | 8.75 (s, 1H), 8.33 (t, J = 3.0 Hz, 1H), 7.48-7.41 (m, 2H), 7.35 (d, J = 2.3 Hz, |
| | CDCl₃ | 1H), 7.26 (d, J = 3.0 Hz, 2H), 7.17-7.11 (m, 2H), 6.77 (d, J = 2.3 Hz, |
| | | 1H), 4.39 (dt, J = 11.7, 3.9 Hz, 1H), 4.04 (td, J = 11.8, 2.3 Hz, |
| | | 1H), 3.91 (s, 3H), 3.52 (d, J = 14.8 Hz, 1H), 3.08 (ddd, J = 15.3, 12.1, |
| | | 3.9 Hz, 1H) |
| 228 | 400 MHz | 8.32 (s, 1H), 8.29 (dd, J = 3.7, 2.3 Hz, 1H), 7.43 (dq, J = 5.2, 3.1 Hz, |
| | CDCl₃ | 3H), 7.34-7.29 (m, 2H), 7.19-7.13 (m, 2H), 6.59 (s, 1H), 4.41 (ddd, J = 11.8, |
| | | 4.2, 2.9 Hz, 1H), 4.16 (s, 3H), 4.01 (ddd, J = 12.9, 11.7, 2.1 Hz, |
| | | 1H), 3.60 (s, 1H), 2.90 (s, 1H) |
| 229 | 400 MHz | 8.41 (s, 1H), 8.31 (dd, J = 4.0, 2.0 Hz, 1H), 7.57-7.49 (m, 1H), |
| | CDCl₃ | 7.49-7.43 (m, 3H), 7.31-7.28 (m, 2H), 7.18-7.12 (m, 2H), 7.06 (dd, J = 5.0, |
| | | 3.7 Hz, 1H), 4.40 (ddd, J = 11.8, 4.3, 3.1 Hz, 1H), 4.01 (ddd, J = 12.8, |
| | | 11.7, 2.2 Hz, 1H), 3.71 (d, J = 14.7 Hz, 1H), 2.95-2.79 (m, 1H). |
| 230 | 400 MHz | 8.34 (s, 1H), 8.30 (dd, J = 3.8, 2.2 Hz, 1H), 7.90 (d, J = 2.8 Hz, 1H), |
| | CDCl₃ | 7.47 (d, J = 2.1 Hz, 1H), 7.45 (d, J = 2.2 Hz, 1H), 7.42 (dd, J = 5.1, 1.4 Hz, |
| | | 1H), 7.33 (dd, J = 5.1, 3.0 Hz, 1H), 7.29 (dddd, J = 3.6, 3.1, 1.7, 0.8 Hz, |
| | | 2H), 7.17-7.12 (m, 2H), 4.41 (ddd, J = 11.7, 4.2, 3.0 Hz, 1H), |
| | | 4.02 (ddd, J = 12.8, 11.7, 2.2 Hz, 1H), 3.71 (d, J = 14.5 Hz, 1H), 2.88 (s, 1H) |
| 231 | 400 MHz | 9.38 (s, 1H), 8.76 (d, J = 2.1 Hz, 1H), 8.36 (dd, J = 3.6, 2.4 Hz, 1H), |
| | CDCl₃ | 8.15 (d, J = 2.1 Hz, 1H), 7.51-7.43 (m, 2H), 7.26 (d, J = 2.0 Hz, 2H), |
| | | 7.15 (dq, J = 8.9, 1.0 Hz, 2H), 4.40 (dt, J = 11.7, 3.8 Hz, 1H), 4.04 (ddd, |
| | | J = 12.3, 11.6, 2.2 Hz, 1H), 3.61 (dt, J = 14.8, 2.8 Hz, 1H), 3.01 (ddd, J = 14.4, |
| | | 12.4, 4.0 Hz, 1H) |
| 232 | 400 MHz | 9.29 (s, 1H), 8.68 (d, J = 4.7 Hz, 1H), 8.37 (dd, J = 3.7, 2.3 Hz, 1H), |
| | CDCl₃ | 7.85 (d, J = 4.7 Hz, 1H), 7.52-7.44 (m, 2H), 7.16 (dq, J = 8.9, 1.0 Hz, |
| | | 2H), 4.42 (dt, J = 11.7, 3.7 Hz, 1H), 4.05 (ddd, J = 12.5, 11.7, 2.2 Hz, |
| | | 1H), 3.60 (d, J = 14.4 Hz, 1H), 3.01 (td, J = 13.6, 4.0 Hz, 1H) |
| 233 | 400 MHz | 8.89 (s, 1H), 8.54 (s, 1H), 8.33 (t, J = 3.0 Hz, 2H), 7.46-7.35 (m, 3H), |
| | CDCl₃ | 7.19 (d, J = 8.2 Hz, 2H), 4.44 (dt, J = 11.8, 3.7 Hz, 1H), 4.09-3.95 (m, |
| | | 1H), 3.51 (s, 1H), 2.98 (s, 1H) |
| 234 | 400 MHz | 9.00-8.55 (m, 2H), 8.32 (d, J = 4.4 Hz, 1H), 8.12-8.06 (m, 1H), |
| | CDCl₃ | 7.42 (dt, J = 7.4, 2.5 Hz, 5H), 7.26-7.20 (m, 2H), 7.16 (d, J = 8.1 Hz, 2H), |
| | | 4.48 (d, J = 11.8 Hz, 1H), 4.07 (t, J = 12.0 Hz, 1H), 3.60-3.40 (m, 1H), |
| | | 3.35-3.15 (m, 1H) |
| 235 | 400 MHz | 8.58 (d, J = 18.9 Hz, 2H), 8.32 (dd, J = 4.3, 1.8 Hz, 1H), 8.08 (s, 1H), |
| | CDCl₃ | 7.65 (d, J = 8.3 Hz, 1H), 7.53 (dd, J = 8.2, 1.3 Hz, 1H), 7.46 (d, J = 8.7 Hz, |
| | | 2H), 7.33 (dd, J = 8.0, 5.2 Hz, 3H), 7.15 (d, J = 8.4 Hz, 2H), 6.58 (t, |
| | | J = 2.3 Hz, 1H), 4.44 (dt, J = 11.7, 3.6 Hz, 1H), 4.06 (td, J = 12.2, 2.2 Hz, |
| | | 1H), 3.55 (s, 1H), 3.12 (s, 1H) |
| 236 | 400 MHz | 8.76 (s, 1H), 8.38 (dd, J = 4.1, 1.8 Hz, 1H), 7.66 (ddd, J = 7.9, 1.4, 0.7 Hz, |
| | CDCl₃ | 1H), 7.53 (dq, J = 8.4, 0.9 Hz, 1H), 7.51-7.45 (m, 3H), 7.41 (ddd, J = 8.4, |
| | | 7.2, 1.3 Hz, 1H), 7.34-7.28 (m, 3H), 7.19-7.13 (m, 2H), |
| | | 4.42 (dt, J = 11.7, 3.7 Hz, 1H), 4.04 (ddd, J = 12.5, 11.7, 2.2 Hz, 1H), |
| | | 3.68 (dt, J = 14.7, 2.7 Hz, 1H), 2.98 (ddd, J = 14.4, 12.6, 4.1 Hz, 1H) |
| 237 | 400 MHz | 8.65 (s, 1H), 8.32 (t, J = 3.0 Hz, 1H), 8.23 (s, 1H), 8.06 (s, 1H), 7.81 (d, |
| | CDCl₃ | J = 8.4 Hz, 1H), 7.71 (s, 1H), 7.51-7.46 (m, 2H), 7.32 (ddddt, J = 3.6, |
| | | 2.1, 1.5, 1.0, 0.5 Hz, 2H), 7.19-7.13 (m, 2H), 4.48-4.41 (m, 1H), |
| | | 4.07 (t, J = 12.0 Hz, 1H), 3.60 (s, 1H), 3.05-2.96 (m, 1H) |
| 238 | 400 MHz | 8.36 (s, 1H), 7.84 (s, 1H), 7.69 (d, J = 7.5 Hz, 2H), 7.50 (d, J = 8.7 Hz, |
| | CDCl₃ | 2H), 7.38-7.30 (m, 2H), 7.26-7.17 (m, 3H), 4.47-4.40 (m, 1H), |
| | | 4.11-4.03 (m, 1H), 3.28 (s, 2H) |
| 239 | 400 MHz | 9.25 (s, 1H), 8.38 (dd, J = 3.4, 2.6 Hz, 1H), 8.17-8.10 (m, 2H), |
| | CDCl₃ | 7.65-7.59 (m, 1H), 7.54-7.47 (m, 2H), 7.27-7.22 (m, 3H), 7.14 (ddt, J = 7.9, |
| | | 2.1, 1.1 Hz, 2H), 6.86 (td, J = 6.7, 1.1 Hz, 1H), 4.41 (dt, J = 11.6, |
| | | 3.9 Hz, 1H), 4.08 (td, J = 11.7, 2.3 Hz, 1H), 3.60-3.48 (m, 1H), |
| | | 3.12 (ddd, J = 14.4, 11.9, 3.9 Hz, 1H) |
| 240 | 400 MHz | 8.84 (d, J = 1.6 Hz, 1H), 8.68 (s, 1H), 8.33 (dd, J = 4.2, 1.7 Hz, 1H), |
| | CDCl₃ | 7.78-7.67 (m, 3H), 7.54 (dd, J = 8.9, 1.7 Hz, 1H), 7.50-7.45 (m, 2H), |
| | | 7.35-7.29 (m, 2H), 7.19-7.14 (m, 2H), 4.43 (ddd, J = 11.7, 4.2, 2.7 Hz, |
| | | 1H), 4.03 (ddd, J = 13.6, 11.7, 2.2 Hz, 1H), 3.77 (dt, J = 14.5, 2.5 Hz, |
| | | 1H), 2.88-2.79 (m, 1H) |
| 241 | 400 MHz | 9.47 (s, 1H), 8.39 (dd, J = 3.3, 2.7 Hz, 1H), 7.66 (s, 2H), 7.53-7.44 (m, |
| | CDCl₃ | 2H), 7.40-7.32 (m, 2H), 7.29 (dtd, J = 2.0, 1.0, 0.5 Hz, 1H), |
| | | 7.22-7.13 (m, 2H), 4.44 (dt, J = 11.7, 3.8 Hz, 1H), 4.09 (td, J = 11.9, 2.3 Hz, 1H), |
| | | 3.55-3.46 (m, 1H), 3.11 (ddd, J = 14.5, 12.1, 4.0 Hz, 1H) |
| 242 | 400 MHz | 9.18 (s, 1H), 8.60 (dd, J = 8.1, 1.6 Hz, 1H), 8.56 (dd, J = 4.7, 1.6 Hz, |
| | CDCl₃ | 1H), 8.50 (dd, J = 4.0, 1.9 Hz, 1H), 7.47 (d, J = 8.7 Hz, 2H), |
| | | 7.41-7.36 (m, 2H), 7.24-7.17 (m, 3H), 4.44 (dt, J = 11.7, 3.5 Hz, 1H), 4.04 (t, J = 11.7 Hz, |
| | | 1H), 3.53-3.44 (m, 1H), 3.16 (d, J = 14.2 Hz, 1H) |
| 243 | 400 MHz | 9.26 (s, 1H), 8.47 (d, J = 0.7 Hz, 1H), 8.37 (ddd, J = 13.3, 4.0, 2.1 Hz, |
| | CDCl₃ | 2H), 7.95 (ddd, J = 9.4, 1.7, 0.7 Hz, 1H), 7.52-7.44 (m, 2H), |
| | | 7.36-7.31 (m, 2H), 7.16 (dt, J = 7.5, 1.2 Hz, 2H), 7.10 (dd, J = 9.3, 4.4 Hz, |
| | | 1H), 4.43 (dt, J = 11.7, 3.8 Hz, 1H), 4.06 (td, J = 12.0, 2.2 Hz, 1H), |
| | | 3.59-3.44 (m, 1H), 3.16 (t, J = 12.7 Hz, 1H) |
| 244 | 400 MHz | 9.45 (s, 1H), 8.76 (dd, J = 7.0, 1.7 Hz, 1H), 8.67-8.60 (m, 2H), |
| | CDCl₃ | 8.36 (dd, J = 4.4, 1.6 Hz, 1H), 7.51-7.44 (m, 2H), 7.36 (s, 2H), 7.18 (d, J = 8.4 Hz, |
| | | 2H), 6.97 (dd, J = 7.0, 4.1 Hz, 1H), 4.41 (dt, J = 11.6, 3.9 Hz, |
| | | 1H), 4.11-4.04 (m, 1H), 3.38 (s, 1H), 3.23 (s, 1H) |
| 245 | 400 MHz | 8.90-8.70 (m, 1H), 8.31-8.21 (m, 1H), 7.45-7.30 (m, 2H), |
| | CDCl₃ | 7.23-7.18 (m, 1H), 7.19-7.00 (m, 4H), 6.84-6.69 (m, 2H), 4.47-4.28 (m, |
| | | 2H), 4.00-3.90 (m, 1H), 3.64-3.40 (m, 2H), 3.08 (td, J = 16.8, 9.4 Hz, |
| | | 1H), 2.94-2.81 (m, 1H) |
| 246 | 400 MHz | 8.78-8.67 (m, 1H), 8.30-8.21 (m, 1H), 7.45-7.36 (m, 1H), |
| | CDCl₃ | 7.26-7.23 (m, 1H), 7.21-7.19 (m, 1H), 7.19-7.06 (m, 4H), 6.95-6.84 (m, |
| | | 2H), 5.12 (ddd, J = 12.5, 10.6, 7.1 Hz, 1H), 4.39-4.25 (m, 1H), |
| | | 4.01-3.88 (m, 1H), 3.62-3.31 (m, 3H), 3.00-2.68 (m, 1H) |
| 247 | 400 MHz | 9.87 (s, 1H), 9.51-9.45 (m, 1H), 8.50 (d, J = 5.5 Hz, 1H), 8.34 (dd, J = 4.0, |
| | CDCl₃ | 1.9 Hz, 1H), 7.87-7.82 (m, 1H), 7.81-7.77 (m, 1H), 7.69 (dddd, J = 21.4, |
| | | 8.4, 6.9, 1.4 Hz, 2H), 7.56-7.48 (m, 2H), 7.27-7.26 (m, 2H), |
| | | 7.18 (ddt, J = 7.8, 2.0, 1.0 Hz, 2H), 4.44 (dt, J = 11.6, 3.9 Hz, 1H), |
| | | 4.11 (td, J = 11.7, 2.3 Hz, 1H), 3.49 (dt, J = 14.9, 3.2 Hz, 1H), 3.28 (ddd, J = 14.4, |
| | | 11.8, 3.9 Hz, 1H) |
| 248 | 400 MHz | 10.04 (s, 1H), 9.20 (t, J = 0.9 Hz, 1H), 8.59 (d, J = 1.0 Hz, 1H), 8.39 (dd, |
| | CDCl₃ | J = 3.6, 2.4 Hz, 1H), 8.07-8.01 (m, 1H), 8.01-7.94 (m, 1H), |
| | | 7.74 (dddd, J = 25.2, 8.1, 6.9, 1.3 Hz, 2H), 7.54-7.44 (m, 2H), |
| | | 7.36-7.29 (m, 2H), 7.21-7.13 (m, 2H), 4.43 (dt, J = 11.6, 3.9 Hz, 1H), 4.09 (td, J = 11.8, |
| | | 2.3 Hz, 1H), 3.52 (d, J = 13.0 Hz, 1H), 3.28-3.16 (m, 1H) |
| 249 | 400 MHz | 9.32 (d, J = 1.0 Hz, 1H), 8.56 (d, J = 6.1 Hz, 1H), 8.26-8.21 (m, 2H), |
| | CDCl₃ | 8.19 (dt, J = 6.1, 0.9 Hz, 1H), 8.10 (dt, J = 8.2, 1.0 Hz, 1H), 7.93 (dd, J = 7.2, |
| | | 1.2 Hz, 1H), 7.65 (dd, J = 8.2, 7.1 Hz, 1H), 7.57-7.50 (m, 2H), |
| | | 7.29-7.27 (m, 2H), 7.20 (dq, J = 8.9, 1.0 Hz, 2H), 4.47 (ddd, J = 11.7, |
| | | 4.2, 2.8 Hz, 1H), 4.06 (ddd, J = 13.0, 11.7, 2.1 Hz, 1H), 3.81 (dt, J = 14.5, |
| | | 2.5 Hz, 1H), 3.05 (ddd, J = 14.5, 13.1, 4.2 Hz, 1H) |
| 250 | 400 MHz | 9.33 (s, 1H), 8.75 (s, 1H), 8.60 (d, J = 5.8 Hz, 1H), 8.34 (dd, J = 4.1, 1.8 Hz, |
| | CDCl₃ | 1H), 8.32-8.30 (m, 1H), 8.07 (dt, J = 8.6, 0.8 Hz, 1H), 8.01 (dd, J = 8.6, |
| | | 1.7 Hz, 1H), 7.78 (d, J = 5.8 Hz, 1H), 7.55-7.48 (m, 2H), |
| | | 7.34-7.28 (m, 2H), 7.17 (dq, J = 7.9, 1.1 Hz, 2H), 4.45 (ddd, J = 11.7, 4.2, 2.8 Hz, |
| | | 1H), 4.06 (ddd, J = 12.8, 11.6, 2.2 Hz, 1H), 3.84 (dt, J = 14.5, 2.5 Hz, |
| | | 1H), 2.89 (ddd, J = 14.4, 13.0, 4.2 Hz, 1H) |
| 251 | 400 MHz | 10.07 (s, 1H), 8.41 (t, J = 3.0 Hz, 1H), 8.34-8.23 (m, 2H), 8.15 (ddt, J = 8.5, |
| | CDCl₃ | 1.4, 0.7 Hz, 1H), 7.91-7.83 (m, 1H), 7.75 (ddd, J = 8.5, 6.9, 1.5 Hz, |
| | | 1H), 7.61 (ddd, J = 8.1, 6.9, 1.2 Hz, 1H), 7.54-7.47 (m, 2H), |
| | | 7.31 (d, J = 3.0 Hz, 2H), 7.21-7.15 (m, 2H), 4.43 (dt, J = 11.6, 3.9 Hz, 1H), |
| | | 4.09 (td, J = 11.8, 2.3 Hz, 1H), 3.48 (d, J = 16.8 Hz, 1H), 3.22 (ddd, J = 14.9, |
| | | 11.9, 3.9 Hz, 1H) |
| 252 | 400 MHz | 9.34 (d, J = 2.3 Hz, 1H), 8.82 (s, 1H), 8.71 (s, 1H), 8.34 (dd, J = 4.0, 2.0 Hz, |
| | CDCl₃ | 1H), 8.25 (d, J = 8.5 Hz, 1H), 8.00-7.93 (m, 1H), 7.85 (ddd, J = 8.5, |
| | | 6.9, 1.4 Hz, 1H), 7.66 (ddd, J = 8.1, 6.9, 1.1 Hz, 1H), |
| | | 7.55-7.47 (m, 2H), 7.37-7.31 (m, 2H), 7.18 (ddt, J = 7.9, 2.0, 1.0 Hz, 2H), |
| | | 4.46 (ddd, J = 11.8, 4.3, 2.8 Hz, 1H), 4.06 (ddd, J = 13.1, 11.8, 2.2 Hz, 1H), |
| | | 3.79 (d, J = 14.6 Hz, 1H), 2.97-2.88 (m, 1H) |
| 253 | 400 MHz | 9.00 (dd, J = 4.3, 1.7 Hz, 1H), 8.73 (s, 1H), 8.39-8.33 (m, 2H), |
| | CDCl₃ | 8.30 (ddd, J = 8.3, 1.7, 0.8 Hz, 1H), 8.21 (d, J = 8.8 Hz, 1H), 8.11 (dd, J = 8.8, |
| | | 2.0 Hz, 1H), 7.55-7.47 (m, 3H), 7.34-7.28 (m, 2H), 7.16 (ddt, J = 7.9, |
| | | 2.1, 1.0 Hz, 2H), 4.45 (ddd, J = 11.7, 4.2, 2.9 Hz, 1H), 4.06 (ddd, J = 13.0, |
| | | 11.7, 2.2 Hz, 1H), 3.83 (dt, J = 14.6, 2.5 Hz, 1H), 2.91 (ddd, J = 14.6, |
| | | 13.0, 4.2 Hz, 1H) |
| 254 | 400 MHz | 9.01 (dd, J = 4.4, 1.7 Hz, 1H), 8.80 (s, 1H), 8.59 (s, 1H), 8.36 (dd, J = 4.1, |
| | CDCl₃ | 1.9 Hz, 1H), 8.29 (d, J = 8.3 Hz, 1H), 8.10 (dd, J = 8.7, 1.8 Hz, 1H), |
| | | 7.94 (d, J = 8.5 Hz, 1H), 7.60-7.49 (m, 3H), 7.34-7.29 (m, 2H), |
| | | 7.17 (ddt, J = 7.9, 2.1, 1.0 Hz, 2H), 4.45 (ddd, J = 11.7, 4.3, 3.1 Hz, 1H), |
| | | 4.07 (ddd, J = 12.7, 11.6, 2.2 Hz, 1H), 3.84-3.74 (m, 1H), 2.94 (td, J = 13.7, |
| | | 4.2 Hz, 1H) |
| 255 | 400 MHz | 9.86 (s, 1H), 9.66 (s, 1H), 8.40 (t, J = 3.0 Hz, 1H), 8.22-8.13 (m, 2H), |
| | CDCl₃ | 7.91-7.79 (m, 2H), 7.52-7.45 (m, 2H), 7.34 (d, J = 3.0 Hz, 2H), |
| | | 7.19 (dq, J = 9.0, 1.0 Hz, 2H), 4.44 (dt, J = 11.7, 3.7 Hz, 1H), 4.12-4.03 (m, |
| | | 1H), 3.53 (d, J = 14.5 Hz, 1H), 3.23-3.11 (m, 1H) |
| 256 | 400 MHz | 8.41 (s, 1H), 8.36-8.31 (m, 1H), 7.63 (d, J = 8.0 Hz, 1H), |
| | CDCl₃ | 7.52-7.45 (m, 2H), 7.38 (d, J = 8.5 Hz, 1H), 7.31 (d, J = 5.8 Hz, 3H), 7.15 (dd, J = 15.7, |
| | | 7.9 Hz, 3H), 6.94 (s, 1H), 4.43 (d, J = 11.3 Hz, 1H), |
| | | 4.11-3.95 (m, 4H), 3.63 (s, 1H), 2.99 (s, 1H) |
| 257 | 400 MHz | 8.43-8.21 (m, 2H), 8.14-8.04 (m, 1H), 7.74 (s, 1H), 7.49 (s, 2H), |
| | CDCl₃ | 7.40-7.32 (m, 3H), 7.30 (dd, J = 7.0, 1.5 Hz, 1H), 7.27-7.23 (m, 1H), |
| | | 7.15 (d, J = 8.4 Hz, 2H), 4.44 (s, 1H), 4.07 (t, J = 11.8 Hz, 1H), 3.84 (s, 3H), |
| | | 3.66 (s, 1H), 3.15-2.95 (m, 1H) |
| 258 | 400 MHz | 8.47 (s, 1H), 8.33 (dd, J = 3.5, 2.5 Hz, 1H), 8.15 (s, 1H), 7.74 (dd, J = 8.6, |
| | CDCl₃ | 1.7 Hz, 1H), 7.49 (d, J = 8.5 Hz, 2H), 7.39-7.29 (m, 3H), 7.16 (d, J = 8.4 Hz, |
| | | 2H), 7.11 (d, J = 3.1 Hz, 1H), 6.56 (dd, J = 3.1, 0.8 Hz, 1H), |
| | | 4.43 (dt, J = 11.7, 3.7 Hz, 1H), 4.12-4.02 (m, 1H), 3.82 (s, 3H), 3.64 (s, |
| | | 1H), 3.07 (s, 1H) |
| 259 | 400 MHz | 8.31 (dd, J = 4.1, 1.9 Hz, 1H), 8.19 (s, 1H), 7.45-7.39 (m, 2H), 7.30 (d, |
| | CDCl₃ | J = 4.7 Hz, 2H), 7.20 (ddd, J = 8.0, 7.4, 1.6 Hz, 1H), 7.18-7.13 (m, |
| | | 2H), 7.11 (dd, J = 7.6, 1.6 Hz, 1H), 7.03 (s, 1H), 6.91 (td, J = 7.5, 1.1 Hz, |
| | | 1H), 6.84 (dt, J = 8.1, 0.8 Hz, 1H), 5.00 (d, J = 1.3 Hz, 2H), |
| | | 4.44-4.35 (m, 1H), 4.00 (ddd, J = 12.9, 11.7, 2.2 Hz, 1H), 3.64 (s, 1H), |
| | | 2.81 (s, 1H) |
| 260 | 400 MHz | 9.53 (s, 1H), 8.39 (dd, J = 3.5, 2.4 Hz, 1H), 7.81 (d, J = 7.9 Hz, 1H), |
| | CDCl₃ | 7.53-7.48 (m, 2H), 7.45-7.39 (m, 2H), 7.38-7.32 (m, 1H), 7.28 (s, |
| | | 2H), 7.17 (d, J = 8.2 Hz, 2H), 4.43 (dt, J = 11.6, 3.8 Hz, 1H), 4.20 (s, |
| | | 3H), 4.08 (td, J = 12.0, 2.2 Hz, 1H), 3.49 (d, J = 11.3 Hz, 1H), |
| | | 3.15-3.03 (m, 1H) |
| 261 | 400 MHz | 8.65 (s, 1H), 8.41 (d, J = 0.9 Hz, 1H), 8.36-8.30 (m, 1H), 7.66 (d, J = 7.0 Hz, |
| | CDCl₃ | 1H), 7.53 (dd, J = 14.4, 8.5 Hz, 3H), 7.44 (dd, J = 8.4, 7.1 Hz, |
| | | 1H), 7.34 (s, 2H), 7.17 (d, J = 8.1 Hz, 2H), 4.46 (dt, J = 11.6, 3.6 Hz, |
| | | 1H), 4.12 (s, 3H), 4.11-4.03 (m, 1H), 3.73 (s, 1H), 3.05 (s, 1H) |
| 262 | 400 MHz | 8.55 (s, 1H), 8.34 (t, J = 3.0 Hz, 1H), 8.25 (s, 1H), 8.07 (d, J = 0.8 Hz, |
| | CDCl₃ | 1H), 7.89 (dd, J = 8.8, 1.4 Hz, 1H), 7.49 (d, J = 8.8 Hz, 2H), 7.42 (d, J = 8.8 Hz, |
| | | 1H), 7.33 (s, 2H), 7.16 (d, J = 8.4 Hz, 2H), 4.47-4.41 (m, 1H), |
| | | 4.10 (s, 3H), 4.09-4.02 (m, 1H), 3.73 (d, J = 7.0 Hz, 1H), 2.96 (s, 1H) |
| 263 | 400 MHz | 8.64 (s, 1H), 8.33 (dd, J = 3.6, 2.4 Hz, 1H), 8.07-8.00 (m, 2H), |
| | CDCl₃ | 7.76 (dd, J = 8.4, 0.8 Hz, 1H), 7.54-7.47 (m, 3H), 7.31 (d, J = 3.2 Hz, 2H), |
| | | 7.16 (dp, J = 7.8, 1.1 Hz, 2H), 4.44 (ddd, J = 11.7, 4.2, 3.0 Hz, 1H), |
| | | 4.13 (s, 3H), 4.06 (ddd, J = 12.8, 11.7, 2.2 Hz, 1H), 3.77 (d, J = 13.6 Hz, 1H), |
| | | 2.95 (s, 1H) |
| 264 | 400 MHz | 8.59 (s, 1H), 8.50 (d, J = 0.8 Hz, 1H), 8.34 (dd, J = 3.6, 2.4 Hz, 1H), |
| | CDCl₃ | 7.86 (dt, J = 8.6, 0.8 Hz, 1H), 7.55-7.48 (m, 3H), 7.36-7.30 (m, 3H), |
| | | 7.19-7.14 (m, 2H), 4.45 (ddd, J = 11.8, 4.1, 3.3 Hz, 1H), 4.26 (d, J = 0.4 Hz, |
| | | 3H), 4.08 (ddd, J = 12.8, 11.7, 2.2 Hz, 1H), 3.75 (s, 1H), 2.96 (d, |
| | | J = 14.7 Hz, 1H) |
| 265 | 400 MHz | 8.61 (s, 1H), 8.36-8.28 (m, 1H), 8.18 (s, 1H), 7.92 (s, 1H), 7.69 (dd, J = 8.8, |
| | CDCl₃ | 0.8 Hz, 1H), 7.56 (dd, J = 8.8, 1.4 Hz, 1H), 7.50 (dd, J = 9.3, 2.4 Hz, |
| | | 2H), 7.30 (d, J = 2.6 Hz, 2H), 7.15 (d, J = 8.1 Hz, 2H), 4.43 (dt, J = 11.7, |
| | | 3.8 Hz, 1H), 4.26 (s, 3H), 4.11-4.01 (m, 1H), 3.73 (d, J = 7.0 Hz, |
| | | 1H), 2.96 (s, 1H) |
| 266 | 400 MHz | 10.66 (s, 1H), 8.36 (dd, J = 4.4, 1.6 Hz, 1H), 8.19 (dd, J = 7.1, 1.1 Hz, |
| | CDCl₃ | 1H), 7.98 (s, 1H), 7.80 (dd, J = 8.3, 1.1 Hz, 1H), 7.57-7.51 (m, 2H), |
| | | 7.34-7.29 (m, 1H) 7.22-7.16 (m, 3H), 4.41 (dt, J = 11.5, 4.1 Hz, 1H), |
| | | 4.18 (s, 3H), 4.12 (td, J = 11.4, 2.3 Hz, 1H), 3.50 (t, J = 11.2 Hz, 1H), |
| | | 3.14 (d, J = 14.1 Hz, 1H) |
| 267 | 400 MHz | 8.57 (s, 1H), 8.35 (dd, J = 4.2, 1.8 Hz, 1H), 7.88-7.81 (m, 2H), 7.77 (s, |
| | CDCl₃ | 1H), 7.52-7.45 (m, 2H), 7.45-7.36 (m, 2H), 7.31 (d, J = 4.3 Hz, 2H), |
| | | 7.16 (d, J = 8.1 Hz, 2H), 4.42 (dt, J = 11.7, 4.0 Hz, 1H), 4.08-3.98 (m, |
| | | 1H), 3.75 (d, J = 14.3 Hz, 1H), 2.88 (s, 1H) |
| 268 | 400 MHz | 8.40 (d, J = 7.9 Hz, 1H), 8.37 (s, 1H), 8.30 (t, J = 3.0 Hz, 1H), 7.97 (s, |
| | CDCl₃ | 2H), 7.89-7.84 (m, 2H), 7.51 (d, J = 8.9 Hz, 2H), 7.48-7.36 (m, 2H), |
| | | 7.17 (d, J = 8.2 Hz, 2H), 4.44 (dt, J = 11.7, 3.7 Hz, 1H), 4.12-3.99 (m, |
| | | 1H), 3.86-3.82 (m, 1H), 2.87 (d, J = 13.0 Hz, 1H) |
| 269 | 400 MHz | 8.77 (s, 1H), 8.44 (d, J = 1.1 Hz, 1H), 8.34 (dd, J = 4.1, 1.8 Hz, 1H), |
| | CDCl₃ | 8.06 (d, J = 1.3 Hz, 2H), 7.59-7.42 (m, 2H), 7.37-7.29 (m, 2H), |
| | | 7.21-7.14 (m, 2H), 4.45 (ddd, J = 11.7, 4.2, 2.8 Hz, 1H), 4.05 (ddd, J = 13.8, |
| | | 11.8, 2.1 Hz, 1H), 3.86-3.82 (m, 1H), 2.87 (d, J = 13.3 Hz, 1H) |
| 270 | 400 MHz | 8.29 (dd, J = 3.3, 2.6 Hz, 1H), 8.12 (s, 1H), 7.49-7.43 (m, 2H), |
| | CDCl₃ | 7.31 (ddddt, J = 4.5, 3.5, 2.0, 1.0, 0.5 Hz, 2H), 7.20-7.14 (m, 2H), 4.42 (dt, J = 11.7, |
| | | 3.6 Hz, 1H), 4.08 (s, 4H), 3.66 (s, 1H), 2.90 (s, 1H), 2.68 (d, J = 15.8 Hz, |
| | | 4H), 1.79 (t, J = 4.6 Hz, 4H) |
| 271 | 400 MHz | 9.81 (s, 1H), 8.75 (s, 2H), 8.33 (s, 1H), 8.11 (d, J = 8.4 Hz, 1H), |
| | CDCl₃ | 7.51 (dd, J = 16.6, 8.6 Hz, 3H), 7.41-7.29 (m, 2H), 7.23-7.14 (m, 2H), |
| | | 7.06 (d, J = 6.4 Hz, 1H), 6.47 (d, J = 7.1 Hz, 1H), 4.43 (d, J = 11.6 Hz, |
| | | 1H), 4.05 (t, J = 11.9 Hz, 1H), 3.40-3.55 (m, 1H), 3.14 (s, 1H) |
| 272 | 400 MHz | 8.39 (d, J = 7.1 Hz, 1H), 8.28 (s, 1H), 8.23-8.11 (m, 2H), |
| | CDCl₃ | 7.77-7.68 (m, 1H), 7.53 (t, J = 7.3 Hz, 1H), 7.46 (d, J = 8.5 Hz, 2H), |
| | | 7.42-7.30 (m, 2H), 7.13-7.10 (m, 2H), 7.18 (d, J = 8.2 Hz, 2H), 4.46 (d, J = 11.7 Hz, |
| | | 1H), 4.03 (t, J = 11.4 Hz, 1H), 3.70 (s, 1H), 2.95 (s, 1H) |
| 273 | 400 MHz | 8.58 (s, 1H), 8.32 (dd, J = 3.3, 2.7 Hz, 1H), 7.96 (s, 1H), 7.90 (d, J = 8.3 Hz, |
| | CDCl₃ | 2H), 7.75-7.70 (m, 2H), 7.49-7.43 (m, 3H), 7.36-7.29 (m, 2H), |
| | | 7.19-7.13 (m, 2H), 4.43 (dt, J = 11.6, 3.6 Hz, 1H), 4.04 (ddd, J = 12.9, |
| | | 11.7, 2.1 Hz, 1H), 3.72 (s, 1H), 2.93 (s, 1H) |
| 274 | 400 MHz | 8.82-8.72 (m, 2H), 8.36 (s, 1H), 7.45 (d, J = 8.4 Hz, 2H), 7.37 (s, 1H), |
| | CDCl₃ | 7.17 (d, J = 8.3 Hz, 3H), 7.00 (s, 1H), 6.90 (d, J = 7.9 Hz, 1H), 4.46 (d, J = 11.8 Hz, |
| | | 1H), 4.01 (d, J = 11.8 Hz, 4H), 3.56 (s, 1H), 2.98 (s, 1H) |
| 275 | 400 MHz | 8.54 (s, 1H), 8.48 (t, J = 1.8 Hz, 1H), 8.32 (dd, J = 3.7, 2.3 Hz, 1H), |
| | CDCl₃ | 8.18 (ddd, J = 7.7, 1.7, 1.1 Hz, 1H), 7.98-7.92 (m, 1H), 7.56 (td, J = 7.8, 0.6 Hz, |
| | | 1H), 7.52-7.45 (m, 2H), 7.32-7.28 (m, 2H), 7.19-7.13 (m, 2H), |
| | | 4.43 (ddd, J = 11.7, 4.2, 3.0 Hz, 1H), 4.05 (ddd, J = 12.8, 11.7, 2.2 Hz, |
| | | 1H), 3.76 (d, J = 14.2 Hz, 1H), 2.98-2.82 (m, 1H), 2.67 (s, 3H) |
| 276 | 400 MHz | 8.60 (s, 1H), 8.33 (dd, J = 3.8, 2.2 Hz, 1H), 8.17-8.10 (m, 2H), |
| | CDCl₃ | 7.95-7.89 (m, 2H), 7.51-7.45 (m, 2H), 7.31 (ddtq, J = 3.1, 2.1, 1.6, 1.0 Hz, |
| | | 2H), 7.18-7.13 (m, 2H), 4.43 (dt, J = 11.7, 3.7 Hz, 1H), 4.04 (ddd, J = 12.8, |
| | | 11.7, 2.2 Hz, 1H), 3.74 (s, 1H), 2.96-2.82 (m, 1H), 2.68 (s, 3H) |
| 277 | 400 MHz | 8.57-8.51 (m, 1H), 8.32 (t, J = 3.0 Hz, 1H), 8.05 (ddd, J = 8.2, 2.3, 1.0 Hz, |
| | CDCl₃ | 1H), 7.94-7.89 (m, 1H), 7.53 (d, J = 7.7 Hz, 1H), 7.50-7.39 (m, |
| | | 3H), 7.35-7.28 (m, 2H), 7.18-7.12 (m, 2H), 4.42 (dt, J = 11.8, 3.6 Hz, |
| | | 1H), 4.04 (ddd, J = 13.8, 11.8, 2.2 Hz, 1H), 3.92 (t, J = 7.0 Hz, 2H), |
| | | 3.68 (s, 1H), 2.98-2.88 (m, 1H), 2.63 (dd, J = 8.5, 7.7 Hz, 2H), 2.18 (qd, J = 8.0, |
| | | 6.8 Hz, 2H) |
| 278 | 400 MHz | 8.31 (dd, J = 3.8, 2.2 Hz, 1H), 8.18 (s, 1H), 7.43-7.38 (m, 2H), |
| | CDCl₃ | 7.35-7.28 (m, 2H), 7.16 (dtt, J = 7.6, 2.0, 1.1 Hz, 2H), 7.02 (s, 1H), |
| | | 6.81-6.75 (m, 2H), 6.67 (dd, J = 2.4, 0.9 Hz, 1H), 4.94 (d, J = 1.3 Hz, 2H), |
| | | 4.44-4.34 (m, 1H), 4.00 (ddd, J = 13.0, 11.7, 2.1 Hz, 1H), 3.77 (s, 3H), |
| | | 3.67-3.52 (m, 1H), 2.88-2.78 (m, 1H) |
| 279 | 300 MHz | 9.24 (s, 1H), 8.35 (dd, J = 3.7, 2.3 Hz, 1H), 8.32 (s, 1H), 7.32-7.41 (m, |
| | CDCl₃ | 1H), 7.26-7.31 (m, 2H), 7.20-7.24 (m, 2H), 4.30-4.55 (m, 3H), |
| | | 3.97-4.14 (m, 1H), 3.46-3.62 (m, 1H), 2.88-3.08 (m, 1H), 1.44 (t, J = 7.2 Hz, 3H). |
| 280 | 300 MHz | 8.56 (s, 1H), 8.45 (t, J = 1.5 Hz, 1H), 8.32 (dd, J = 3.8, 2.2 Hz, 1H), |
| | CDCl₃ | 8.16 (dt, J = 7.7, 1.3 Hz, 1H), 8.01 (dd, J = 7.9, 1.5 Hz, 1H), 7.52 (t, J = 7.7 Hz, |
| | | 1H), 7.29-7.41 (m, 3H), 7.19-7.25 (m, 2H), 4.40-4.47 (m, 1H), |
| | | 3.98-4.12 (m, 1H), 3.94 (s, 3H), 3.75 (d, J = 14.6 Hz, 1H), |
| | | 2.74-2.96 (m, 1H) |
| 281 | 300 MHz, | 9.28 (d, J = 1.3 Hz, 1H), 8.58 (dd, J = 8.2, 2.2 Hz, 1H), 8.17-8.34 (m, |
| | MeOH-d₄ | 2H), 7.33-7.51 (m, 4H), 7.22-7.31 (m, 1H), 4.43 (dt, J = 11.9, 4.1 Hz, |
| | | 1H), 4.09 (td, J = 11.5, 2.4 Hz, 1H), 3.33-3.41 (m, 1H), 3.13 (ddd, J = 14.7, |
| | | 11.2, 3.8 Hz, 1H) |
| 282 | 300 MHz, | 9.39 (s, 1H), 9.30 (dd, J = 2.0, 0.7 Hz, 1H), 8.62 (dd, J = 8.2, 2.2 Hz, |
| | MeOH-d₄ | 1H), 8.28 (t, J = 2.9 Hz, 1H), 8.24 (dd, J = 8.2, 0.7 Hz, 1H), 7.46 (dd, J = 11.9, |
| | | 2.3 Hz, 1H), 7.33-7.43 (m, 3H), 7.23-7.30 (m, 1H), 4.42 (dt, J = 11.8, |
| | | 4.1 Hz, 1H), 4.08 (td, J = 11.6, 2.3 Hz, 1H), 3.32-3.43 (m, 1H), |
| | | 3.13 (ddd, J = 14.8, 11.2, 3.9 Hz, 1H) |
| 283 | 300 MHz, | 8.67 (br. s., 1H), 8.25 (d, J = 3.4 Hz, 1H), 8.00 (d, J = 8.6 Hz, 1H), |
| | MeOH-d₄ | 7.87 (d, J = 8.0 Hz, 1H), 7.32-7.58 (m, 4H), 7.19 (d, J = 9.2 Hz, 1H), |
| | | 4.32-4.52 (m, 1H), 4.03-4.20 (m, 1H), 3.04-3.22 (m, 1H), 2.89 (m, 1H) |
| 284 | 300 MHz, | 8.24 (dd, J = 3.6, 2.2 Hz, 1H), 7.96 (d, J = 2.2 Hz, 1H), 7.76 (d, J = 8.6 Hz |
| | MeOH-d₄ | 1H), 7.33-7.48 (m, 4H), 7.17-7.28 (m, 1H), 7.04 (dd, J = 8.5, 2.6 Hz, |
| | | 1H), 4.31-4.46 (m, 1H), 4.02-4.18 (m, 1H), 3.16-3.24 (m, 2H) |
| 285 | 300 MHz, | 8.05-8.12 (m, 2H), 8.01-8.04 (m, 1H), 7.82-7.92 (m, 1H), |
| | MeOH-d₄ | 7.45-7.53 (m, 1H), 7.20-7.34 (m, 2H), 7.07-7.18 (m, 3H), 4.29-4.43 (m, 1H), |
| | | 3.98-4.10 (m, 1H), 3.96 (s, 3H), 2.82-2.94 (m, 2H) |
| 286 | 300 MHz, | 8.04 (dd, J = 4.4, 1.5 Hz, 1H), 7.92-7.98 (m, 2H), 7.63-7.74 (m, 2H), |
| | MeOH-d₄ | 7.22-7.33 (m, 2H), 7.06-7.20 (m, 3H), 4.23-4.45 (m, 1H), |
| | | 3.97-4.10 (m, 1H), 3.95 (s, 3H), 2.78-2.93 (m, 2H) |
| 287 | 400 MHz, | 13.03 (br. s., 1H), 9.01 (br. s., 1H), 8.10 (d, J = 3.1 Hz, 1H), |
| | DMSO-d₆ | 7.63-7.92 (m, 3H), 7.52 (d, J = 12.9 Hz, 1H), 7.16-7.44 (m, 4H), 4.26-4.42 (m, |
| | | 1H), 4.20 (br. s., 1H), 3.22 (br. s., 1H), 2.82-2.98 (m, 1H), 2.51 (s, 3H). |
| 288 | 400 MHz, | 8.19 (dd, J = 4.3, 1.8 Hz, 1H), 7.81 (d, J = 8.0 Hz, 1H), 7.61-7.72 (m, |
| | MeOH-d₄ | 4H), 7.51-7.61 (m, 2H), 7.24-7.40 (m, 2H), 4.32-4.46 (m, 1H), |
| | | 4.01-4.13 (m, 1H), 3.34-3.43 (m, 2H), 2.99-3.10 (m, 1H), 2.58 (s, 3H). |
| 289 | 400 MHz, | 13.27 (br. s., 1H), 9.10 (br. s., 1H), 8.13 (br. s., 1H), 8.02 (br. s., 1H), |
| | DMSO-d₆ | 7.79 (d, J = 5.7 Hz, 2H), 7.45-7.60 (m, 2H), 7.34-7.45 (m, 1H), |
| | | 7.11-7.34 (m, 3H), 5.81 (d, J = 17.4 Hz, 1H), 5.38 (d, J = 10.8 Hz, 1H), |
| | | 4.34 (m, 1H), 4.13 (m, 1H), 3.35 (m, 1H), 2.84 (m, 1H). |
| 290 | 400 MHz, | 13.02 (br. s., 1H), 9.15 (s, 1H), 8.12 (dd, J = 4.3, 1.4 Hz, 1H), |
| | DMSO-d₆ | 7.85-7.90 (m, 1H), 7.84 (s, 1H), 7.77 (d, J = 8.0 Hz, 1H), 7.35-7.60 (m, 6H), |
| | | 7.21-7.34 (m, 3H), 4.35 (m, 1H), 4.16 (m, 1H), 3.32 (m, 1H), 2.84 (m, 1H). |
| 291 | 400 MHz, | 13.07 (br. s., 1H), 8.98 (s, 1H), 8.13 (dd, J = 4.2, 1.7 Hz, 1H), |
| | DMSO-d₆ | 7.76-7.82 (m, 1H), 7.67-7.75 (m, 2H), 7.42-7.58 (m, 2H), 7.19-7.35 (m, 3H), |
| | | 4.34 (ddd, J = 11.1, 7.9, 2.7 Hz, 1H), 4.13 (ddd, J = 11.2, 7.9, 2.6 Hz, 1H), |
| | | 3.27 (td, J = 7.1, 2.9 Hz, 1H), 2.93 (q, J = 7.4 Hz, 2H), 2.86 (ddd, J = 14.2, |
| | | 7.7, 2.7 Hz, 1H), 1.16 (t, J = 7.4 Hz, 3H) |
| 292 | 400 MHz, | 13.13 (br. s., 1H), 9.05 (s, 1H), 8.15 (dd, J = 4.2, 1.7 Hz, 1H), |
| | DMSO-d₆ | 7.80-7.85 (m, 1H), 7.74 (dd, J = 8.0, 1.6 Hz, 1H), 7.66 (d, J = 8.2 Hz, 1H), |
| | | 7.46-7.59 (m, 2H), 7.21-7.36 (m, 3H), 4.35 (ddd, J = 11.2, 7.9, 2.8 Hz, 1H), |
| | | 4.08-4.18 (m, 1H), 3.67 (dt, J = 13.7, 6.9 Hz, 1H), 3.20-3.32 (m, 1H), |
| | | 2.81-2.93 (m, 1H), 1.22 (d, J = 6.8 Hz, 6H). |
| 293 | 400 MHz, | 13.02 (br. s., 1H), 8.96 (s, 1H), 8.12 (dd, J = 4.2, 1.7 Hz, 1H), 7.77 (d, J = 8.0 Hz, |
| | DMSO-d₆ | 1H), 7.71 (dd, J = 8.1, 1.7 Hz, 1H), 7.64 (d, J = 1.4 Hz, 1H), |
| | | 7.43-7.56 (m, 2H), 7.24-7.34 (m, 2H), 7.17-7.24 (m, 1H), 4.22-4.46 (m, |
| | | 1H), 4.12 (ddd, J = 11.2, 8.0, 2.7 Hz, 1H), 3.17-3.28 (m, 1H), |
| | | 2.76-2.97 (m, 3H), 1.67-1.91 (m, 1H), 0.83 (dd, J = 6.7, 2.0 Hz, 6H). |
| 294 | 400 MHz, | 8.94 (s, 1H), 8.15 (d, J = 2.5 Hz, 1H), 7.82 (d, J = 8.2 Hz, 1H), |
| | DMSO-d₆ | 7.46-7.62 (m, 2H), 7.21-7.37 (m, 3H), 7.04 (s, 1H), 6.97 (d, J = 8.6 Hz, 1H), |
| | | 4.35 (t, J = 8.2 Hz, 1H), 4.15 (t, J = 8.1 Hz, 1H), 3.13-3.27 (m, 1H), |
| | | 2.75-3.05 (m, 1H), 2.87 (s, 3H). |
| 295 | 400 MHz, | 10.44 (s, 1H), 9.04 (s, 1H), 8.40 (d, J = 1.6 Hz, 1H), 8.13 (dd, J = 4.2, |
| | DMSO-d₆ | 1.7 Hz, 1H), 7.83-7.92 (m, 1H), 7.60 (dd, J = 8.2, 1.6 Hz, 1H), |
| | | 7.42-7.56 (m, 2H), 7.25-7.33 (m, 2H), 7.19-7.25 (m, 1H), 4.34 (ddd, J = 11.2, 7.8, |
| | | 2.8 Hz, 1H), 4.12 (ddd, J = 11.3, 8.1, 2.7 Hz, 1H), 3.84 (s, 3H), |
| | | 3.24 (ddd, J = 14.3, 7.9, 2.8 Hz, 1H), 2.89 (ddd, J = 14.3, 7.5, 2.8 Hz, 1H), |
| | | 2.10 (s, 3H). |
| 296 | 300 MHz, | 8.88 (s, 1H), 8.20 (d, J = 4.5 Hz, 1H), 8.13 (dd, J = 4.1, 1.8 Hz, 1H), |
| | DMSO-d₆ | 7.69 (s, 1H), 7.65 (d, J = 8.0 Hz, 1H), 7.42-7.57 (m, 2H), 7.20-7.39 (m, |
| | | 4H), 4.27-4.41 (m, 1H), 4.00-4.20 (m, 1H), 3.19-3.29 (m, 1H), |
| | | 2.82-2.96 (m, 1H), 2.75 (d, J = 4.5 Hz, 3H), 2.35 (s, 3H). |
| 297 | 300 MHz, | 8.85 (s, 1H), 8.12 (dd, J = 4.0, 1.8 Hz, 1H), 7.85 (s, 1H), 7.68 (s, 1H), |
| | DMSO-d₆ | 7.59-7.67 (m, 1H), 7.42-7.56 (m, 2H), 7.19-7.34 (m, 4H), 4.33 (ddd, J = 11.3, |
| | | 8.1, 2.7 Hz, 1H), 4.05-4.21 (m, 1H), 3.19-3.29 (m, 1H), |
| | | 2.81-2.96 (m, 1H), 2.32 (s, 3H), 1.36 (s, 9H). |
| 298 | 400 MHz, | 11.27 (br. s., 1H), 8.99 (s, 1H), 8.12 (br. s., 1H), 7.82 (d, J = 8.0 Hz, 1H), |
| | DMSO-d₆ | 7.41-7.60 (m, 2H), 7.37 (s, 1H), 7.19-7.35 (m, 4H), 4.33 (t, J = 8.6 Hz, |
| | | 1H), 4.12 (t, J = 8.9 Hz, 1H), 3.20-3.25 (m, 1H), 2.76-2.92 (m, 1H). |
| 299 | 400 MHz, | 12.43 (br. s., 1H), 8.33 (s, 1H), 8.09 (br. s., 1H), 7.59 (s, 1H), |
| | DMSO-d₆ | 7.39-7.52 (m, 2H), 7.32 (s, 1H), 7.22-7.30 (m, 2H), 7.19 (d, J = 8.6 Hz, 1H), |
| | | 4.24-4.36 (m, 1H), 4.15 (br. s., 1H), 3.84 (s, 3H), 3.24 (dd, J = 14.0, 5.8 Hz, |
| | | 1H), 2.75-2.89 (m, 1H) |
| 300 | 400 MHz, | 13.11 (br. s., 1H), 9.00 (br. s., 1H), 8.12 (br. s., 1H), 7.71 (d, J = 7.6 Hz, |
| | DMSO-d₆ | 1H), 7.64 (d, J = 7.8 Hz, 1H), 7.43-7.57 (m, 2H), 7.26-7.37 (m, 3H), |
| | | 7.22 (d, J = 8.4 Hz, 1H), 4.24-4.40 (m, 1H), 4.11 (t, J = 8.7 Hz, 1H), |
| | | 3.11-3.28 (m, 1H), 2.75-2.92 (m, 1H), 2.62 (br. s., 1H), 0.97 (d, J = 7.6 Hz, |
| | | 2H), 0.74 (br. s., 2H). |
| 301 | 400 MHz, | 11.82 (br. s., 1H), 11.73 (br. s., 1H), 8.47 (s, 1H), 8.11 (d, J = 3.1 Hz, |
| | DMSO-d₆ | 1H), 7.41-7.59 (m, 2H), 7.09-7.35 (m, 4H), 4.31 (t, J = 8.9 Hz, 1H), |
| | | 4.06-4.19 (m, 1H), 3.24 (dd, J = 13.4, 5.4 Hz, 1H), 2.78-2.88 (m, 1H), |
| | | 2.38 (s, 3H). |
| 302 | 400 MHz, | 8.88 (s, 1H), 8.16 (dd, J = 4.0, 1.9 Hz, 1H), 8.02 (br. s., 1H), 7.81 (d, J = 8.2 Hz, |
| | DMSO-d₆ | 1H), 7.46-7.57 (m, 2H), 7.44 (d, J = 1.4 Hz, 1H), 7.26-7.36 (m, |
| | | 2H), 7.20-7.26 (m, 1H), 7.05 (dd, J = 8.2, 1.6 Hz, 1H), 4.30-4.41 (m, |
| | | 1H), 4.12 (ddd, J = 11.3, 8.5, 2.5 Hz, 1H), 3.18 (ddd, J = 14.4, 8.3, 2.9 Hz, |
| | | 2H), 2.98 (ddd, J = 14.2, 7.1, 2.7 Hz, 1H), 0.71-0.83 (m, 2H), |
| | | 0.41-0.50 (m, 2H). |
| 303 | 400 MHz, | 8.56 (s, 1H), 8.12 (dd, J = 4.3, 1.8 Hz, 1H), 7.43-7.53 (m, 2H), |
| | DMSO-d₆ | 7.21-7.35 (m, 4H), 4.34 (ddd, J = 11.2, 8.6, 2.6 Hz, 1H), 4.09-4.22 (m, 1H), |
| | | 3.63-3.71 (m, 3H), 3.08-3.21 (m, 1H), 2.85 (ddd, J = 14.1, 8.5, 2.8 Hz, 1H), |
| | | 2.46 (s, 3H). |
| 304 | 400 MHz, | 8.94 (s, 1H), 8.13 (dd, J = 4.2, 1.7 Hz, 1H), 7.42-7.56 (m, 2H), |
| | DMSO-d₆ | 7.24-7.34 (m, 2H), 7.18-7.24 (m, 1H), 6.85 (s, 1H), 6.81-6.84 (m, 1H), 4.32 (ddd, J = 11.2, |
| | | 7.9, 2.8 Hz, 1H), 4.12 (ddd, J = 11.2, 8.0, 2.7 Hz, 1H), 3.25 (ddd, |
| | | J = 14.2, 7.7, 2.7 Hz, 1H), 2.84 (s, 3H), 2.76-2.91 (ddd, J = 14.2, 7.7, 2.7 Hz, |
| | | 1H). |
| 305 | 400 MHz, | 8.38 (s, 1H), 8.09 (dd, J = 4.2, 1.7 Hz, 1H), 7.63 (d, J = 1.8 Hz, 1H), |
| | DMSO-d₆ | 7.42-7.49 (m, 2H), 7.41 (d, J = 2.0 Hz, 1H), 7.22-7.30 (m, 2H), 7.19 (dd, |
| | | J = 8.8, 1.2 Hz, 1H), 4.29 (ddd, J = 11.3, 8.7, 2.5 Hz, 1H), 4.10-4.20 (m, |
| | | 1H), 3.85 (s. 3H), 3.74 (s, 3H), 3.18-3.30 (m, 1H), 2.80 (ddd, J = 14.2, |
| | | 8.7, 3.1 Hz, 1H). |
| 306 | 400 MHz, | 12.75 (br. s., 1H), 12.25 (br. s., 1H), 8.69-8.78 (m, 1H), 8.13 (dd, J = 4.3, |
| | DMSO-d₆ | 1.6 Hz, 1H), 7.44-7.56 (m, 2H), 7.24-7.36 (m, 2H), 7.14-7.24 (m, 1H), |
| | | 6.66-6.75 (m, 2H), 4.30 (ddd, J = 11.2, 8.1, 2.6 Hz, 1H), 4.05-4.16 (m, |
| | | 1H), 3.29-3.33 (m, 1H), 2.70-2.81 (m, 1H). |
| 307 | 300 MHz, | 8.97 (s, 1H), 8.31 (dd, J = 8.2, 2.2 Hz, 1H), 8.26 (dd, J = 3.8, 2.2 Hz, |
| | MeOH-d₄ | 1H), 8.07-8.19 (m, 3H), 7.66-7.76 (m, 1H), 7.56-7.66 (m, 2H), |
| | | 7.34-7.48 (m, 4H), 7.18-7.28 (m, 1H), 4.42 (dt, J = 12.1, 4.3 Hz, 1H), |
| | | 4.09 (td, J = 11.4, 2.5 Hz, 1H), 3.25 (dd, J = 4.7, 2.5 Hz, 1H), 3.02-3.16 (m, |
| | | 1H). |
| 308 | 300 MHz, | 8.23 (t, J = 3.0 Hz, 1H), 7.65 (dd, J = 1.8, 0.7 Hz, 1H), 7.31-7.49 (m, |
| | MeOH-d₄ | 4H), 7.17-7.25 (m, 1H), 7.12 (dd, J = 3.5, 0.7 Hz, 1H), 6.58 (dd, J = 3.5, |
| | | 1.8 Hz, 1H), 4.34-4.45 (m, 1H), 3.98-4.10 (m, 1H), |
| | | 3.19-3.29 (m, 1H), 3.11 (dd, J = 10.9, 3.7 Hz, 1H) |
| 309 | 300 MHz, | 8.23 (t, J = 1.6 Hz, 1H), 8.19 (dd, J = 3.9, 2.0 Hz, 1H), 7.89-8.02 (m, |
| | MeOH-d₄ | 2H), 7.55 (t, J = 7.7 Hz, 1H), 7.27-7.49 (m, 4H), 7.19-7.27 (m, 1H), |
| | | 4.34-4.47 (m, 1H), 4.10 (td, J = 10.9, 2.6 Hz, 1H), 3.30-3.40 (m, 1H), |
| | | 2.97-3.12 (m, 1H), 2.92 (s, 3H) |
| 310 | 400 MHz, | 9.72 (s, 1H), 8.17-8.40 (m, 2H), 7.94 (d, J = 7.82 Hz, 1H), |
| | DMSO-d₆ | 7.47-7.62 (m, 2H), 7.33-7.45 (m, 2H), 7.25 (dd, J = 1.17, 8.61 Hz, 1H), |
| | | 4.33-4.59 (m, 1H), 3.88-4.14 (m, 1H), 3.49 (td, J = 2.01, 12.62 Hz, 1H), |
| | | 2.81-2.97 (m, 1H), 2.73 (s, 3H) |
| 311 | 400 MHz, | 8.82 (s, 1H), 8.12 (dd, J = 4.2, 1.7 Hz, 1H), 7.52 (s, 2H), 7.45-7.54 (m, |
| | DMSO-d₆ | 2H), 7.11-7.32 (m, 3H), 4.33 (ddd, J = 11.2, 8.3, 2.6 Hz, 1H), |
| | | 4.07-4.22 (m, 1H), 3.20-3.29 (m, 1H), 2.86 (ddd, J = 14.1, 8.0, 2.7 Hz, 1H), |
| | | 2.28 (s, 6H) |
| 312 | 400 MHz, | 9.00 (d, J = 1.37 Hz, 1 H), 8.88 (s, 1 H), 8.39 (dd, J = 8.02, 1.96 Hz, 1 H), |
| | DMSO-d₆ | 8.14 (dd, J = 4.30, 1.57 Hz, 1 H), 8.09 (d, J = 8.02 Hz, 1 H), 7.29-7.41 (m, |
| | | 5 H), 7.23 (d, J = 8.80 Hz, 1 H), 4.20-4.30 (m, 2 H), 3.53-3.62 (m, 1 |
| | | H), 2.68-2.79 (m, 1 H). |
| 313 | 400 MHz, | 9.11 (d, J = 1.37 Hz, 1 H), 8.43 (dd, J = 8.02, 1.96 Hz, 1 H), 8.27 (t, J = 2.93 Hz |
| | MeOH-d₄ | 1 H), 8.11 (d, J = 8.22 Hz, 1 H), 7.65 (t, J = 7.92 Hz, 1 H), 7.44 (d, |
| | | J = 12.52 Hz, 1 H), 7.30-7.39 (m, 3 H), 4.35-4.45 (m, 1 H), 4.11 (td, |
| | | J = 11.20, 2.45 Hz, 1 H), 3.33-3.39 (m, 1 H), 3.15 (ddd, J = 14.57, |
| | | 10.66, 3.52 Hz, 1 H). |
| 314 | 400 MHz, | 8.26 (t, J = 3.0 Hz, 1H), 7.99 (d, J = 8.0 Hz, 1H), 7.74 (s, 1H), 7.69 (dd, |
| | DMSO-d₆ | J = 8.0, 1.3 Hz, 1H), 7.45-7.54 (m, 1H), 7.25-7.43 (m, 4H), |
| | | 4.35-4.49 (m, 1H), 4.06-4.17 (m, 1H), 4.04 (s, 3H), 3.32-3.38 (m, 1H), |
| | | 3.08 (ddd, J = 14.6, 11.0, 3.8 Hz, 1H) |
| 315 | 400 MHz, | 8.89 (s, 1H), 8.09-8.17 (m, 2H), 7.98 (dd, J = 8.7, 2.2 Hz, 1H), |
| | DMSO-d₆ | 7.43-7.56 (m, 2H), 7.25-7.36 (m, 2H), 7.22 (d, J = 9.0 Hz, 1H), 7.16 (d, J = 8.8 Hz, |
| | | 1H), 4.28-4.38 (m, 1H), 4.09-4.19 (m, 1H), 3.86 (s, 3H), |
| | | 3.20-3.32 (m, 1H), 2.80-2.90 (m, 1H) |
| 316 | 400 MHz, | 12.85 (br. s., 1H), 9.71 (s, 1H), 8.39 (d, J = 2.3 Hz, 1H), 8.29 (dd, J = 3.3, |
| | DMSO-d₆ | 2.5 Hz, 1H), 8.04 (dd, J = 8.6, 2.3 Hz, 1H), 7.63 (dd, J = 12.3, 2.2 Hz, |
| | | 1H), 7.45-7.58 (m, 1H), 7.36 (d, J = 1.0 Hz, 1H), 7.36 (s, 1H), |
| | | 7.31 (dt, J = 8.7, 1.1 Hz, 1H), 7.26 (d, J = 8.8 Hz, 1H), 4.31-4.50 (m, 1H), |
| | | 4.06-4.18 (m, 1H), 4.00 (s, 3H), 3.37-3.52 (m, 1H), 2.88 (ddd, J = 14.3, |
| | | 10.3, 3.5 Hz, 1H). |
| 317 | 300 MHz, | 8.09 (dd, J = 4.7, 1.4 Hz, 1H), 7.96 (d, J = 8.6 Hz, 2H), 7.65 (d, J = 8.6 Hz, |
| | MeOH-d₄ | 2H), 7.28-7.39 (m, 3H), 7.15-7.26 (m, 2H), 4.29-4.47 (m, 1H), |
| | | 4.02-4.16 (m, 1H), 2.71-2.85 (m, 2H) |
| 318 | 400 MHz, | 9.60 (s, 1H), 9.05 (d, J = 1.2 Hz, 1H), 8.41 (dd, J = 8.0, 2.0 Hz, 1H), |
| | DMSO-d₆ | 8.28-8.36 (m, 1H), 8.10 (d, J = 8.0 Hz, 1H), 7.67 (d, J = 2.3 Hz, 1H), |
| | | 7.61 (d, J = 8.6 Hz, 1H), 7.42 (br. s., 2H), 7.36 (dd, J = 8.6, 2.3 Hz, 1H), |
| | | 4.40 (s, 1H), 4.04 (s, 1H), 3.51 (d, J = 14.9 Hz, 1H), 2.92 (ddd, J = 14.5, |
| | | 10.8, 3.4 Hz, 1H) |
| 319 | 300 MHz, | 8.08 (dd, J = 3.9, 2.0 Hz, 1H), 7.82 (d, J = 8.0 Hz, 1H), 7.59 (s, 1H), |
| | MeOH-d₄ | 7.54 (dd, J = 8.1, 1.4 Hz, 1H), 7.30-7.41 (m, 2H), 7.20-7.27 (m, 2H), |
| | | 7.13-7.20 (m, 2H), 4.28 (dt, J = 11.9, 4.1 Hz, 1H), 3.92 (td, J = 11.3, 2.3 Hz, |
| | | 1H), 3.25-3.34 (m, 1H), 2.78-2.92 (m, 1H), 2.45-2.54 (m, 3H). |
| 320 | 400 MHz, | 12.45 (br. s., 1H), 12.07 (br. s., 1H), 8.33 (s, 1H), 8.09 (dd, J = 4.1, 1.8 Hz, |
| | DMSO-d₆ | 1H), 7.52 (dd, J = 3.1, 1.8 Hz, 1H), 7.41-7.49 (m, 2H), 7.15-7.31 (m, |
| | | 4H), 4.30 (ddd, J = 11.2, 8.5, 2.5 Hz, 1H), 4.07-4.19 (m, 1H), |
| | | 3.21-3.27 (m, 1H), 2.81 (ddd, J = 14.1, 8.4, 2.9 Hz, 1H). |
| 321 | 400 MHz, | 8.85 (s, 1H), 8.15 (dd, J = 4.0, 1.5 Hz, 1H), 7.73 (d, J = 8.4 Hz, 1H), |
| | DMSO-d₆ | 7.45-7.59 (m, 2H), 7.21-7.40 (m, 3H), 7.14 (s, 1H), 6.88 (dd, J = 8.3, 1.1 Hz, |
| | | 1H), 4.35 (t, J = 8.2 Hz, 1H), 4.15 (t, J = 8.1 Hz, 1H), 3.13-3.27 (m, |
| | | 1H), 2.75-3.05 (m, 1H). |
| 322 | 300 MHz, | 8.35 (dd, J = 3.7, 2.3 Hz, 1H), 8.29 (s, 1H), 7.86 (s, 1H), 7.62 (d, J = 8.2 Hz, |
| | CDCl₃ | 1H), 7.47 (dd, J = 8.3, 1.3 Hz, 1H), 7.36-7.43 (m, 2H), |
| | | 7.28-7.36 (m, 2H), 7.15-7.23 (m, 1H), 7.10 (s, 1H), 4.40-4.48 (m, 1H), |
| | | 3.98-4.10 (m, 1H), 3.95 (s, 3H), 3.17-3.41 (m, 2H). |
| 323 | 300 MHz, | 10.94 (s, 1H), 9.02 (s, 1H), 8.72 (d, J = 1.6 Hz, 1H), 8.14 (dd, J = 3.9, |
| | DMSO-d₆ | 1.9 Hz, 1H), 7.98 (d, J = 8.2 Hz, 1H), 7.44-7.61 (m, 3H), 7.19-7.36 (m, |
| | | 3H), 4.29-4.44 (m, 1H), 4.06-4.20 (m, 1H), 3.16-3.26 (m, 1H), |
| | | 2.80-2.99 (m, 1H), 2.13 (s, 3H). |
| 324 | 400 MHz, | 12.43 (br. s., 1H), 8.34 (s, 1H), 8.10 (dd, J = 4.2, 1.7 Hz, 1H), 7.60 (d, J = 1.8 Hz, |
| | DMSO-d₆ | 1H), 7.42-7.49 (m, 2H), 7.32 (d, J = 2.0 Hz, 1H), |
| | | 7.22-7.30 (m, 2H), 7.17-7.22 (m, 1H), 4.30 (ddd, J = 11.2, 8.7, 2.4 Hz, 1H), |
| | | 4.09-4.20 (m, 1H), 3.84 (s, 3H), 3.25 (ddd, J = 14.3, 6.8, 2.5 Hz, 1H), |
| | | 2.82 (ddd, J = 14.1, 8.6, 2.9 Hz, 1H). |
| 325 | 400 MHz, | 13.15 (br. s., 1H), 12.01 (br. s., 1H), 8.88 (s, 1H), 8.15 (dd, J = 4.1, 1.8 Hz, |
| | DMSO-d₆ | 1H), 7.91 (s, 1H), 7.67 (d, J = 8.4 Hz, 1H), 7.42-7.62 (m, 3H), |
| | | 7.22-7.38 (m, 3H), 7.11 (s, 1H), 4.29-4.43 (m, 1H), 4.08-4.23 (m, 1H), |
| | | 3.19-3.28 (m, 1H), 2.88-3.04 (m, 1H) |
| 326 | 300 MHz, | 10.43 (s, 1H), 9.90 (dd, J = 2.0, 0.7 Hz, 1H), 9.27 (dd, J = 8.1, 2.1 Hz, |
| | MeOH-d₄ | 1H), 9.12 (t, J = 3.0 Hz, 1H), 8.88-9.05 (m, 1H), 8.52 (d, J = 8.5 Hz, |
| | | 2H), 8.42 (d, J = 8.3 Hz, 2H), 8.21 (d, J = 2.9 Hz, 2H), 5.23 (dt, J = 11.8, |
| | | 4.1 Hz, 1H), 4.74-4.95 (m, 1H), 4.20-4.36 (m, 1H), 3.81 (ddd, J = 14.6, |
| | | 10.9, 3.7 Hz, 1H) |
| 327 | 300 MHz, | 9.13 (d, J = 1.3 Hz, 1H), 8.44 (dd, J = 8.0, 1.9 Hz, 1H), 8.24 (dd, J = 3.5, |
| | MeOH-d₄ | 2.5 Hz, 1H), 8.10 (d, J = 8.0 Hz, 1H), 7.45-7.56 (m, 2H), |
| | | 7.32-7.39 (m, 2H), 7.23-7.32 (m, J = 8.2 Hz, 2H), 4.40 (dt, J = 11.7, 3.9 Hz, 1H), |
| | | 3.96-4.08 (m, 1H), 3.12-3.27 (m, 2H) |
| 328 | 300 MHz, | 9.05 (d, J = 1.4 Hz, 1H), 8.38 (dd, J = 8.1, 2.2 Hz, 1H), 8.18-8.28 (m, |
| | MeOH-d₄ | 2H), 7.53-7.61 (m, 1H), 7.37-7.53 (m, 3H), 7.17-7.28 (m, 1H), |
| | | 4.49 (ddd, J = 11.8, 6.6, 3.3 Hz, 1H), 4.19 (ddd, J = 11.8, 9.0, 2.6 Hz, 1H), |
| | | 3.21-3.29 (m, 1H), 2.87 (ddd, J = 14.4, 6.6, 2.8 Hz, 1H) |
| 329 | 300 MHz, | 8.44 (s, 1H), 8.22 (dd, J = 3.9, 1.9 Hz, 1H), 7.30-7.54 (m, 4H), 7.23 (d, J = 8.8 Hz, |
| | MeOH-d₄ | 1H), 4.31-4.53 (m, 1H), 3.99-4.19 (m, 1H), 3.15-3.27 (m, 1H), |
| | | 2.98-3.15 (m, 1H). |
| 330 | 400 MHz, | 9.61 (s, 1H), 8.83 (dd, J = 4.9, 0.7 Hz, 1H), 8.37-8.43 (m, 1H), |
| | DMSO-d₆ | 8.32 (dd, J = 3.5, 2.5 Hz, 1H), 8.03 (dd, J = 5.0, 1.6 Hz, 1H), 7.47-7.66 (m, |
| | | 2H), 7.35-7.47 (m, 2H), 7.27 (dt, J = 8.7, 1.1 Hz, 1H), 4.33-4.48 (m, |
| | | 1H), 3.97-4.13 (m, 1H), 3.45-3.58 (m, 1H), 2.82-3.03 (m, 1H) |
| 331 | 300 MHz, | 8.17 (d, J = 3.9 Hz, 1H), 7.92 (t, J = 7.4 Hz, 1H), 7.50-7.68 (m, 3H), |
| | MeOH-d₄ | 7.42-7.50 (m, 1H), 7.25-7.42 (m, 2H), 7.13 (d, J = 8.0 Hz, 1H), |
| | | 4.41 (td, J = 7.5, 3.4 Hz, 1H), 4.04-4.24 (m, 1H), 3.02-3.18 (m, 1H), |
| | | 2.78 (dd, J = 14.4, 4.0 Hz, 1H). |
| 332 | 400 MHz, | 13.02 (br. s., 1H), 8.96 (s, 1H), 8.12 (dd, J = 4.2, 1.7 Hz, 1H), 7.83 (d, J = 8.0 Hz, |
| | DMSO-d₆ | 1H), 7.72 (s, 1H), 7.69 (d, J = 8.2 Hz, 1H), 7.44-7.57 (m, |
| | | 2H), 7.19-7.32 (m, 3H), 4.34 (ddd, J = 11.2, 7.9, 2.6 Hz, 1H), |
| | | 4.13 (ddd, J = 11.1, 7.8, 2.6 Hz, 1H), 3.27 (td, J = 7.0, 2.7 Hz, 1H), 2.86 (ddd, |
| | | J = 14.1, 7.8, 2.5 Hz, 1H), 2.54 (s, 3H) |
| 333 | 400 MHz, | 9.55 (s, 1H), 8.90 (s, 1H), 8.30 (dd, J = 2.45, 3.42 Hz, 1H), |
| | DMSO-d₆ | 7.87-8.04 (m, 2H), 7.45-7.64 (m, 2H), 7.33-7.45 (m, 2H), 7.18-7.28 (m, 1H), |
| | | 4.39 (td, J = 4.06, 11.83 Hz, 1H), 3.94-4.16 (m, 1H), 3.42-3.55 (m, |
| | | 2H), 2.77-2.97 (m, 3H) |
| 334 | 400 MHz, | 12.00 (1 H, br. s.), 11.96 (1 H, br. s.), 8.64 (1 H, s), 8.17 (1 H, dd, J = 4.1, |
| | DMSO-d₆ | 1.6 Hz), 7.51-7.60 (2 H, m), 7.37-7.45 (2 H, m), 7.29-7.37 (2 H, m), |
| | | 7.27 (1 H, d, J = 8.6 Hz), 4.37 (1 H, ddd, J = 11.2, 8.3, 2.5 Hz), |
| | | 4.12-4.25 (1 H, m), 3.24-3.35 (1 H, m), 2.88 (1 H, ddd, J = 14.1, 8.2, 2.7 Hz). |
| 335 | 400 MHz, | 11.93 (1 H, br. s.), 8.60 (1 H, s), 8.12 (1 H, dd, J = 4.1, 1.6 Hz), |
| | DMSO-d₆ | 7.41-7.57 (3 H, m), 7.17-7.35 (4 H, m), 4.34 (1 H, ddd, J = 11.2, 8.3, 2.6 Hz), |
| | | 4.11-4.20 (1 H, m), 3.76 (3 H, s), 3.15-3.24 (1 H, m), 2.85 (1 H, ddd, |
| | | J = 14.2, 8.3, 2.9 Hz). |
| 336 | 400 MHz, | 13.39 (br. s., 1H), 9.09 (s, 1H), 8.13 (dd, J = 3.9, 1.9 Hz, 1H), 7.93 (d, J = 3.9 Hz, |
| | DMSO-d₆ | 1H), 7.67 (d, J = 3.9 Hz, 1H), 7.44-7.58 (m, 2H), |
| | | 7.24-7.34 (m, 2H), 7.20 (d, J = 8.6 Hz, 1H), 4.25-4.38 (m, 1H), 4.09 (t, J = 8.5 Hz, |
| | | 1H), 3.24 (d, J = 8.2 Hz, 1H), 2.74-2.86 (m, 1H) |
| 337 | 400 MHz, | 9.03 (s, 1H), 8.15 (dd, J = 4.5, 1.6 Hz, 1H), 7.65 (d, J = 8.2 Hz, 1H), |
| | DMSO-d₆ | 7.43-7.58 (m, 4H), 7.36 (dd, J = 8.3, 1.3 Hz, 1H), 7.31 (dd, J = 8.3, 4.4 Hz, |
| | | 1H), 7.19-7.25 (m, 1H), 4.36 (ddd, J = 11.2, 7.9, 2.6 Hz, 1H), |
| | | 4.07-4.20 (m, 1H), 3.85 (s, 3H), 3.26 (ddd, J = 14.2, 7.7, 2.7 Hz, 1H), |
| | | 2.83 (ddd, J = 14.2, 7.8, 2.8 Hz, 1H) |

### Prophetic Examples

The following examples can be made as shown in Scheme 2. The synthesis may be further adapted into an asymmetric synthesis as shown in Scheme 3.

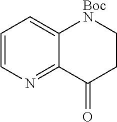 [SMILES: CC(C)(C)OC(=O)N1CCC(=O)c2ncccc21]

### Intermediate 52

### tert-butyl 4-oxo-3,4-dihydro-1,5-naphthyridine-1(2H)-carboxylate

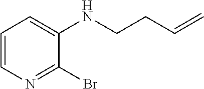

Step 1. 2-bromo-N-(but-3-en-1-yl)pyridin-3-amine

To a solution of 3-amino-2-bromo-pyridine (1 eq, Apollo Scientific Ltd., CAS#39856-58-1) and DMF (0.2 M) is added 60% NaH (1.2 eq). After stirring at room temperature for 30 minutes, the reaction is treated with 4-bromobut-1-ene (1 eq.). Reaction progress is monitored by TLC until judged complete. The reaction mixture is diluted with water and the aqueous solution is extracted with EtOAc. The organic layer is dried over Na₂SO₄, and concentrated. The product thus obtained is purified by column chromatography to afford the title compound.

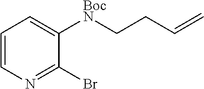 [SMILES: C=CCCN(C(=O)OC(C)(C)C)c1cccnc1Br]

Step 2. tert-butyl (2-bromopyridin-3-yl)(but-3-en-1-yl)carbamate

To a solution of 2-bromo-N-(but-3-en-1-yl)pyridin-3-amine (1 eq.) and DCM (0.2 M) is added (Boc)₂O (1.5 eq.). The solution is stirred at room temperature until judged complete by TLC. The solution is concentrated in vacuo and purified by column chromatography to give the title compound.

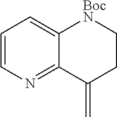 [SMILES: C=C1CCN(C(=O)OC(C)(C)C)c2cccnc21]

Step 3. tert-butyl 4-methylene-3,4-dihydro-1,5-naphthyridine-1(2H)-carboxylate

To a solution of tert-butyl (2-bromopyridin-3-yl)(but-3-en-1-yl)carbamate (1 eq.) in DMF (0.2 M), PPh₃ (0.25 eq.), Pd(OAc)₂ (0.1 eq), and KOAc (5 eq.), is added, under an argon atmosphere, tetraethyl ammonium chloride hydrate (2 eq.). The flask is purged with argon for 15 min, and the resulting reaction mixture is stirred at 110° C. for 16 h. The reaction progress is monitored by TLC. The reaction mixture is diluted with EtOAc and saturated NaHCO₃ solution. The organic layer is separated and dried over Na₂SO₄, and concentrated. The compound that may be thus obtained is purified by column chromatography to afford the title compound.

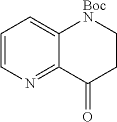 [SMILES: CC(C)(C)OC(=O)N1CCC(=O)c2ncccc21]

Step 4. tert-butyl 4-oxo-3,4-dihydro-1,5-naphthyridine-1(2H)-carboxylate

To a solution of tert-butyl 4-methylene-3,4-dihydro-1,5-naphthyridine-1(2H)-carboxylate (1 eq.) in a mixture of solvents (MeOH:CHCl₃) is added a catalytic amount of NaHCO₃. The reaction mixture is cooled to −78° C. and purged with O₃. Reaction progress is monitored by TLC. After the reaction is judged complete, the reaction mixture is quenched with dimethyl sulfide (5 eq.) at −78° C. The resulting mixture is then stirred for 12 h at ambient temperature. The reaction mixture is diluted with EtOAc and water. The organic layer is washed with water, dried over Na₂SO₄, and concentrated in vacuo. The product that may be thus obtained is purified by column chromatography to give the title compound.

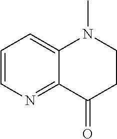

### Intermediate 53: 1-methyl-2,3-dihydro-1,5-naphthyridin-4(1H)-one

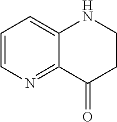 [SMILES: O=C1CCNc2cccnc21]

Step 1: 2,3-dihydro-1,5-naphthyridin-4(1H)-one

To a solution of tert-butyl 4-oxo-3,4-dihydro-1,5-naphthyridine-1(2H)-carboxylate (1 eq.) and 2-MeTHF (0.2 M) is added 4M HCl in dioxane (5 eq.). The reaction is monitored by TLC. The reaction is diluted with EtOAc and washed with saturated NaHCO₃. The organic solution is dried over MgSO₄ and concentrated in vacuo. The compound that may be thus obtained is purified by column chromatography to give the title compound.

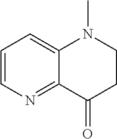

Step 2. 1-methyl-2,3-dihydro-1,5-naphthyridin-4(1H)-one

To a solution of 2,3-dihydro-1,5-naphthyridin-4(1H)-one (1 eq.) and DMF (0.2 M) is added NaH (60%, 1.25 eq). The reaction is stirred for 1 h, and is then treated with iodomethane (1.3 eq.). The reaction is monitored by TLC. The reaction is quenched with water and extracted with EtOAc. The combined organic layers are then dried over MgSO₄, concentrated in vacuo, and purified by column chromatography to give the title compound.

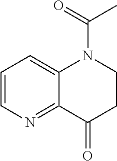

### Intermediate 54

### 1-acetyl-2,3-dihydro-1,5-naphthyridin-4(1H)-one

To a solution of 2,3-dihydro-1,5-naphthyridin-4(1H)-one (1 eq.) and DCM (0.2 eq.) is added DIPEA (2.2 eq.) and acetyl chloride (1.2 eq.). The reaction is monitored by TLC. The reaction is washed with water, brine, dried over MgSO₄, concentrated in vacuo, and purified by column chromatography to give the title compound.

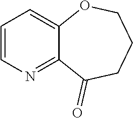 [SMILES: O=C1CCCOc2cccnc21]

### Intermediate 55

### tert-butyl 7,8-dihydrooxepino[3,2-b]pyridin-9(6H)-one

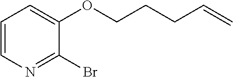

Step 1. 2-bromo-3-(pent-4-en-1-yloxy)pyridine

Diethyl azodicarboxylate (1 eq.) is added dropwise to a stirring mixture of 2-bromo-3-hydroxypyridine (0.92 eq,), pent-4-en-1-ol (0.92 eq), and PPh₃ (1.1 eq) in THF (0.2 M) at 0° C. under a N₂ atmosphere. The reaction mixture is warmed to 50° C. in an oil bath. Reaction progress is monitored by TLC until judged complete. The reaction mixture is cooled to ambient temperature and diluted with saturated NaHCO₃ solution. The aqueous solution is extracted with EtOAc, and the organic layer is dried over Na₂SO₄ and concentrated. The compound that may be thus obtained is purified by column chromatography to afford the title compound.

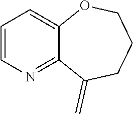

Step 2. 9-methylene-6,7,8,9-tetrahydrooxepino[3,2-b]pyridine

To a solution of 2-bromo-3-(pent-4-en-1-yloxy)pyridine (1 eq.) in DMF (0.2 M), PPh₃ (0.25 eq.), Pd(OAc)₂ (0.1 eq), and KOAc (5 eq.), is added, under an argon atmosphere, tetraethyl ammonium chloride hydrate (2 eq.). The flask is purged with argon for 15 min, and the resulting reaction mixture is stirred at 110° C. for 16 h. The reaction progress is monitored by TLC. The reaction mixture is diluted with EtOAc and saturated NaHCO₃ solution. The organic layer is separated, dried over Na₂SO₄, and concentrated. The compound that may be thus obtained is purified by column chromatography to afford the title compound.

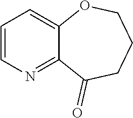 [SMILES: O=C1CCCOc2cccnc21]

Step 3. tert-butyl 4-oxo-3,4-dihydro-1,5-naphthyridine-1(2H)-carboxylate

To a solution of 9-methylene-6,7,8,9-tetrahydrooxepino[3,2-b]pyridine (1 eq.) in a mixture of solvents (MeOH:CHCl₃) is added a catalytic amount of NaHCO₃. The reaction mixture is cooled to −78° C. and purged with O₃. Reaction progress is monitored by TLC. After the reaction is judged complete, the reaction mixture is quenched with dimethyl sulfide (5 eq.) at −78° C. The resulting mixture is stirred for 12 h at ambient temperature. The reaction mixture is diluted with EtOAc and water. The organic layer is washed with water, dried over Na₂SO₄, and concentrated in vacuo. The compound that may be thus obtained is purified by column chromatography to give the title compound.

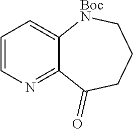 [SMILES: CC(C)(C)OC(=O)N1CCCC(=O)c2ncccc21]

### Intermediate 56

### tert-butyl 9-oxo-6,7,8,9-tetrahydro-5H-pyrido[3,2-b]azepine-5-carboxylate

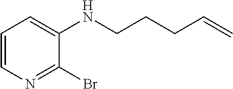

Step 1. 2-bromo-N-(pent-4-en-1-yl)pyridin-3-amine

To a solution of 3-amino-2-bromo-pyridine (1 eq, Apollo Scientific Ltd., CAS#39856-58-1) and DMF (0.2 M) is added 60% NaH (1.2 eq). After stirring at room temperature for 30 minutes, the reaction is treated with 5-bromopent-1-ene (1 eq.). Reaction progress is monitored by TLC until judged complete. The reaction mixture is diluted with water and the aqueous solution is extracted with EtOAc. The organic layer is dried over Na₂SO₄, and concentrated. The product thus obtained is purified by column chromatography to afford the title compound.

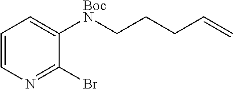 [SMILES: C=CCCCN(C(=O)OC(C)(C)C)c1cccnc1Br]

Step 2. tert-butyl (2-bromopyridin-3-yl)(pent-4-en-1-yl)carbamate

To a solution of 2-bromo-N-(pent-4-en-1-yl)pyridin-3-amine (1 eq.) and DCM (0.2 M) is added (Boc)₂O (1.5 eq.). The solution is stirred at room temperature until judged complete by TLC. The solution is concentrated in vacuo and purified by column chromatography to give the title compound.

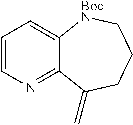 [SMILES: C=C1CCCN(C(=O)OC(C)(C)C)c2cccnc21]

Step 3. tert-butyl9-methylene-6,7,8,9-tetrahydro-5H-pyrido[3,2-b]azepine-5-carboxylate

To a solution of tert-butyl (2-bromopyridin-3-yl)(pent-4-en-1-yl)carbamate (1 eq.) in DMF (0.2 M), PPh₃ (0.25 eq.), Pd(OAc)₂ (0.1 eq), and KOAc (5 eq.), is added, under an argon atmosphere, tetraethyl ammonium chloride hydrate (2 eq.). The flask is purged with argon for 15 min, and the resulting reaction mixture is stirred at 110° C. for 16 h. The reaction progress is monitored by TLC. The reaction mixture is diluted with EtOAc and saturated NaHCO₃ solution. The organic layer is separated, dried over Na₂SO₄, and concentrated. The product thus obtained is purified by column chromatography to afford the title compound.

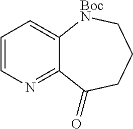 [SMILES: CC(C)(C)OC(=O)N1CCCC(=O)c2ncccc21]

Step 4. tert-butyl 9-oxo-6,7,8,9-tetrahydro-5H-pyrido[3,2-b]azepine-5-carboxylate

To a solution of tert-butyl 9-methylene-6,7,8,9-tetrahydro-5H-pyrido[3,2-b]azepine-5-carboxylate (1 eq.) in a mixture of solvents (MeOH:CHCl₃) is added a catalytic amount of NaHCO₃. The reaction mixture is cooled to −78° C. and purged with O₃. Reaction progress is monitored by TLC. After the reaction is judged complete, the reaction mixture is quenched with dimethyl sulfide (5 eq.) at −78° C. The resulting mixture is stirred for 12 h at ambient temperature. The reaction mixture is diluted with EtOAc and water. The organic layer is washed with water, dried over Na₂SO₄, and concentrated in vacuo. The compound that may be thus obtained is purified by column chromatography to give the title compound.

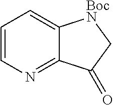 [SMILES: CC(C)(C)OC(=O)N1CC(=O)c2ncccc21]

### Intermediate 57

### tert-butyl 3-oxo-2,3-dihydro-1H-pyrrolo[3,2-b]pyridine-1-carboxylate

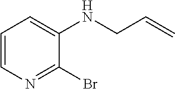

Step 1. N-allyl-2-bromopyridin-3-amine

To a solution of 3-amino-2-bromo-pyridine (1 eq, Apollo Scientific Ltd., CAS#39856-58-1) and DMF (0.2 M) is added 60% NaH (1.2 eq). After stirring at room temperature for 30 minutes, the reaction is treated with allyl bromide (1 eq.). Reaction progress is monitored by TLC until judged complete. The reaction mixture is diluted with water and the aqueous solution is extracted with EtOAc. The organic layer is dried over Na₂SO₄, and concentrated. The product thus obtained is purified by column chromatography to afford the title compound.

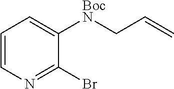 [SMILES: C=CCN(C(=O)OC(C)(C)C)c1cccnc1Br]

Step 2. tert-butyl allyl(2-bromopyridin-3-yl)carbamate

To a solution of N-allyl-2-bromopyridin-3-amine (1 eq.) and DCM (0.2 M) is added (Boc)₂O (1.5 eq.). The solution is stirred at room temperature until judged complete by TLC. The solution is concentrated in vacuo and purified by column chromatography to give the title compound.

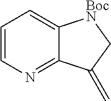 [SMILES: C=C1CN(C(=O)OC(C)(C)C)c2cccnc21]

Step 3. tert-butyl 3-methylene-2,3-dihydro-1H-pyrrolo[3,2-b]pyridine-1-carboxylate

To a solution of tert-butyl allyl(2-bromopyridin-3-yl)carbamate (1 eq.) in DMF (0.2 M), PPh₃ (0.25 eq.), Pd(OAc)₂ (0.1 eq), and KOAc (5 eq.), is added, under an argon atmosphere, tetraethyl ammonium chloride hydrate (2 eq.). The flask is purged with argon for 15 min, and the resulting reaction mixture is stirred at 110° C. for 16 h. The reaction progress is monitored by TLC. The reaction mixture is diluted with EtOAc and saturated NaHCO₃ solution. The organic layer is separated and dried over Na₂SO₄, and concentrated. The product that may be thus obtained is purified by column chromatography to afford the title compound.

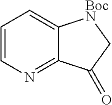 [SMILES: CC(C)(C)OC(=O)N1CC(=O)c2ncccc21]

Step 4. tert-butyl 3-oxo-2,3-dihydro-1H-pyrrolo[3,2-b]pyridine-1-carboxylate

To a solution of tert-butyl 3-methylene-2,3-dihydro-1H-pynolo[3,2-b]pyridine-1-carboxylate (1 eq.) in a mixture of solvents (MeOH:CHCl₃) is added a catalytic amount of NaHCO₃. The reaction mixture is cooled to −78° C. and purged with O₃. Reaction progress may be monitored by TLC. After the reaction is judged complete, the reaction mixture is quenched with dimethyl sulfide (5 eq.) at −78° C. The resulting mixture is stirred for 12 h at ambient temperature. The reaction mixture is diluted with EtOAc and water. The organic layer is washed with water, dried over Na₂SO₄, and concentrated in vacuo. The resulting compound is purified by column chromatography to give the title compound.

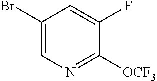 [SMILES: Fc1cc(Br)cnc1OC(F)(F)F]

### Intermediate 58

### 5-bromo-3-fluoro-2-(trifluoromethoxy)pyridine

Reference: Eur. J. Org. Chem. 2010, 6043-6066.

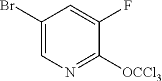

Step 1. 5-bromo-3-fluoro-2-(trichloromethoxy)pyridine

Thiophosgene (1 eq.) in chloroform is added dropwise at 0° C. to a solution of 5-bromo-3-fluoro-2-hydroxypyridine (1 eq.) in aqueous NaOH (5%). The reaction mixture is vigorously stirred for 2 h at 0° C. before being extracted with chloroform. The combined organic layers are washed with dilute hydrochloric acid, water, and dried with Na₂SO₄ before being filtered. The filtrate is saturated with chlorine at 25° C. until the reaction mixture begins to warm up. After 2 h at 25° C., excess chlorine is again added until a yellow solution is obtained. After 24 h at 25° C., excess chlorine is removed with a stream of Ar gas and the solution is concentrated. The pale yellow oil that may be thus obtained may be distilled under vacuum to afford the title compound.

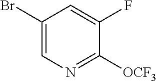 [SMILES: Fc1cc(Br)cnc1OC(F)(F)F]

Step 2. 5-bromo-3-fluoro-2-(trifluoromethoxy)pyridine

5-Bromo-3-fluoro-2-(trichloromethoxy)pyridine (1 eq.) is added dropwise at 120° C. to a mixture of SbF₃ (2.0 eq.) and SbCl₅ (0.15 eq.). The resulting mixture is stirred for 3 h at 140° C. GC monitoring indicates 100% conversion and disappearance of the OCF₂Cl by product. The mixture is then cooled to 0° C. and dissolved in DCM. The solution is neutralized with saturated NaHCO₃ and potassium fluoride and the aqueous layer is extracted with DCM. The combined organic layers may be dried over Na₂SO₄ and the solvent distilled off. The product that may be thus obtained is distilled under vacuum to afford the title compound.

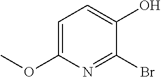

### Intermediate 59

### 2-bromo-6-methoxypyridin-3-ol

To a 0° C. solution of 2-bromo-6-chloropyridin-3-ol (1 eq.) and DMF (0.2 M) is added NaOMe (2.2 eq.). The solution is allowed to warm to room temperature as the cooling bath expires and allowed to stir at room temperature for 24 h. The reaction is quenched with saturated NH₄Cl and diluted with water. The aqueous solution is extracted with EtOAc. The combined EtOAc layers are dried over MgSO₄, concentrated in vacuo, and purified by column chromatography to give the title compound.

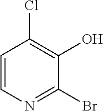

### Intermediate 60

### 2-bromo-4-chloropyridin-3-ol

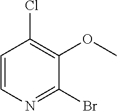

Step 1. 2-bromo-4-chloro-3-methoxypyridine

Phosphorus oxychloride (10 eq.) and 3-methoxy-2-bromo-4(1H)-pyridone (1 eq.) are stirred at 90° C. for 18 h, concentrated in vacuo, and cooled to 20° C. The residue is treated with ice water and adjusted to pH 12 with 40% NaOH, and the product is extracted into DCM. The residue obtained on evaporation of the combined extracts is distilled at reduced pressure to afford the title compound.

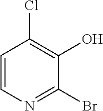

Step 2. 2-bromo-4-chloropyridin-3-ol

To a 0° C. solution of 2-bromo-4-chloro-3-methoxypyridine (1 eq.) and DCM (0.2 M) is added BBr₃ (1.2 eq.) dropwise. The reaction is allowed to warm to room temperature as the cooling bath expires. The reaction is stirred at room temperature until judged complete by TLC. The reaction is washed with water, brine, dried over MgSO₄, and concentrated in vacuo. The material is purified by column chromatography to give the title compound.

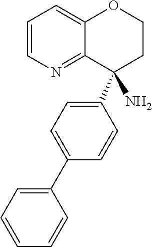 [SMILES: N[C@]1(c2ccc(-c3ccccc3)cc2)CCOc2cccnc21]

### Intermediate 60a

### (S)-4-([1,1′-biphenyl]-4-yl)-3,4-dihydro-2H-pyrano[3,2-b]pyridin-4-amine

The title compound is made in an analogous fashion to Intermediate 36 using phenylboronic acid.

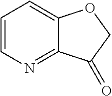

### Intermediate 60b

### furo[3,2-b]pyridin-3(2H)-one

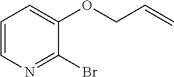

Step 1. 3-(allyloxy)-2-bromopyridine

Diethyl azodicarboxylate (1 eq.) is added dropwise to a stirring mixture of 2-bromo-3-hydroxypyridine (0.92 eq,), allyl alcohol (0.92 eq), and PPh₃ (1.1 eq) in THF (0.2 M) at 0° C. under a N₂ atmosphere. The reaction mixture is warmed to 50° C. in an oil bath. Reaction progress is monitored by TLC until judged complete. The reaction mixture is cooled to ambient temperature and diluted with saturated NaHCO₃ solution. The aqueous solution is extracted with EtOAc, and the organic layer is dried over Na₂SO₄ and concentrated. The product that may be thus obtained is purified by column chromatography to afford the title compound.

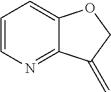

Step 2. 3-methylene-2,3-dihydrofuro[3,2-b]pyridine

To a solution of 3-(allyloxy)-2-bromopyridine (1 eq.) in DMF (0.2 M), PPh₃ (0.25 eq.), Pd(OAc)_{z} (0.1 eq), and KOAc (5 eq.), is added, under an argon atmosphere, tetraethyl ammonium chloride hydrate (2 eq.). The flask is purged with argon for 15 min, and the resulting reaction mixture is stirred at 110° C. for 16 h. The reaction progress is monitored by TLC. The reaction mixture is diluted with EtOAc and saturated NaHCO₃ solution. The organic layer is separated and dried over Na₂SO₄, and concentrated. The product that may be thus obtained is purified by column chromatography to afford the title compound.

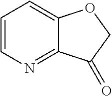

Step 3. furo[3,2-b]pyridin-3(2H)-one

To a solution of 3-methylene-2,3-dihydrofuro[3,2-b]pyridine (1 eq.) in a mixture of solvents (MeOH:CHCl₃) is added a catalytic amount of NaHCO₃. The reaction mixture is cooled to −78° C. and purged with O₃. Reaction progress is monitored by TLC. After the reaction is judged complete, the reaction mixture is quenched with dimethyl sulfide (5 eq.) at −78° C. The resulting mixture is stirred for 12 h at ambient temperature. The reaction mixture is diluted with EtOAc and water. The organic layer is washed with water, dried over Na₂SO₄, and concentrated in vacuo. The compound that may be thus obtained is purified by column chromatography to give the title compound.

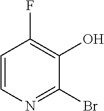

### Intermediate 61: 2-bromo-4-fluoropyridin-3-ol

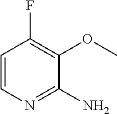

Step 1. 4-fluoro-3-methoxypyridin-2-amine

To a solution of 2-amino-4-fluoropyridin-3-ol (1 eq.) and DCM (1 M) is added iodomethane (1.1 eq.), Adogen® 464 (methyltrialkyl(C₈-C₁₀)ammonium chloride (0.1 eq.), and 40% NaOH (same volume as DCM). The reaction is stirred at room temperature until judged complete by TLC. The DCM layer is separated and the aqueous layer is extracted with DCM. The combined DCM layers are dried over MgSO₄, and concentrated in vacuo. The material is purified by column chromatography to give the title compound.

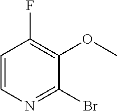

Step 2. 2-bromo-4-fluoro-3-methoxypyridine

To a 0° C. solution of 4-fluoro-3-methoxypyridin-2-amine (1 eq.) and 48% HBr (10 eq.) is added bromine (3 eq.) dropwise, followed by the addition of 40% NaNO₂ (5.5 eq.). The reaction is stirred at 0″C until judged complete by TLC. The reaction mixture is adjusted to pH 13 with 50% NaOH (aq). The aqueous solution is extracted with EtOAc. The combined EtOAc layers are dried over MgSO₄, concentrated in vacuo, and purified by column chromatography to give the title compound.

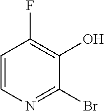

Step 3. 2-bromo-4-fluoropyridin-3-ol

To a 0° C. solution of 2-bromo-4-fluoro-3-methoxypyridine (1 eq.) and DCM (0.2 M) is added BBr₃ (1.2 eq.) dropwise. The reaction is allowed to warm to room temperature as the cooling bath expires. The reaction is then stirred at room temperature until judged complete by TLC. The reaction is washed with water, brine, dried over MgSO₄, and concentrated in vacuo. The material is purified by column chromatography to give the title compound.

### General Procedure for the synthesis of Ketone Intermediate

The following ketone intermediates can be synthesized from the listed starting pyridine in an analogous fashion to Intermediate 1 using Intermediate 1 steps 1-3.

TABLE 18A
Prophetic Example Intermediate reaction conditions.

| Intermediate | | |
|---|---|---|
| No. | Starting Pyridine | Ketone Intermediate |
| 61a¹ | 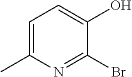 | 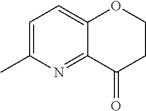 |
| 61b² | 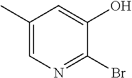 | 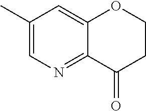 |
| 61c³ | 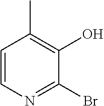 | 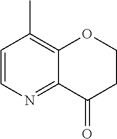 |
| 61d⁴ | 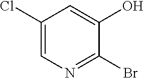 | 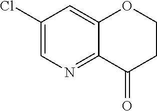 [SMILES: O=C1CCOc2cc(Cl)cnc21] |
| 61e | Intermediate 60 | 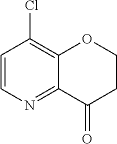 [SMILES: O=C1CCOc2c(Cl)ccnc21] |
| 61f | 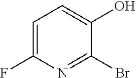 | 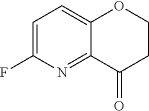 [SMILES: O=C1CCOc2ccc(F)nc21] |
| 61g⁵ | 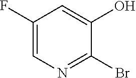 | 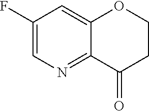 [SMILES: O=C1CCOc2cc(F)cnc21] |
| 61h | Intermediate 61 | 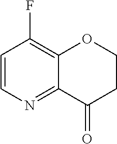 [SMILES: O=C1CCOc2c(F)ccnc21] |
| 61i | Intermediate 59 | 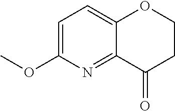 |
| 61j⁶ | 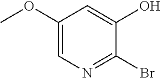 | 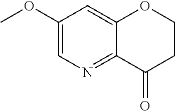 |
| 61k⁷ | 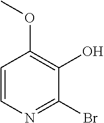 | 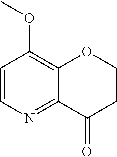 |

¹The starting pyridine is commercially available from: Frontier Scientific, CAS#23003-35-2.
²The starting pyridine is commercially available from: Capot Chemical Co. Ltd., CAS#1003711-30-5.
³The starting pyridine is commercially available from: Kingston Chemistry, Cat#KST-D1099.
⁴The starting pyridine is commercially available from: Kingston Chemistry, CAS#127561-70-0, Cat#KST-F0151.
⁵The starting pyridine is commercially available from: Capot Chemical Co. Ltd., CAS#1093758-87-2.
⁶The starting pyridine is commercially available from: Cheminstock Ltd., Cat#C0410.
⁷The starting pyridine is commercially available from: Chemoraga, Inc., Cat#B00939.

### General Procedures for the Synthesis of Amine Intermediates

The following amine intermediates can be synthesized from the listed intermediate in an analogous fashion to Intermediate 17.

TABLE 18B
Prophetic Example Intermediate reaction conditions and structures.

| Inter- | | | | |
|---|---|---|---|---|
| mediate | | | | |
| No. | Ketone Intermediate | Grignard | Amine Product | Intermediate Name |
| 62 | 1, Step 3 | 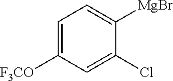 | 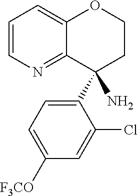 [SMILES: N[C@]1(c2ccc(OC(F)(F)F)cc2Cl)CCOc2cccnc21] | (S)-4-(2-chloro-4- (trifluorometh- oxy)phenyl)-3,4- dihydro-2H- pyrano[3,2- b]pyridin-4-amine |
| 63 | 1, Step 3 | 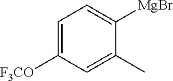 | 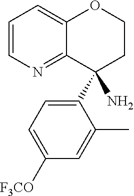 [SMILES: Cc1cc(OC(F)(F)F)ccc1[C@@]1(N)CCOc2cccnc21] | (S)-4-(2-methyl-4- (trifluorometh- oxy)phenyl)-3,4- dihydro-2H- pyrano[3,2- b]pyridin-4-amine |
| 64 | 1, Step 3 | 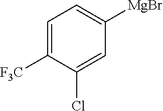 | 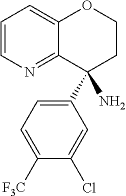 [SMILES: N[C@]1(c2ccc(C(F)(F)F)c(Cl)c2)CCOc2cccnc21] | (S)-4-(3-chloro-4- (trifluoromethyl) phenyl)-3,4- dihydro-2H- pyrano[3,2- b]pyridin-4-amine |
| 65 | 1, Step 3 | 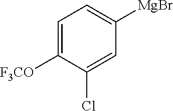 | 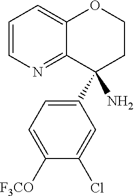 [SMILES: N[C@]1(c2ccc(OC(F)(F)F)c(Cl)c2)CCOc2cccnc21] | (S)-4-(3-chloro-4- (trifluorometh- oxy)phenyl)-3,4- dihydro-2H- pyrano[3,2- b]pyridin-4-amine |
| 66 | 61a | 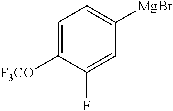 | 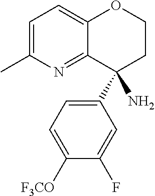 [SMILES: Cc1ccc2c(n1)[C@@](N)(c1ccc(OC(F)(F)F)c(F)c1)CCO2] | (S)-4-(3-fluoro-4- (trifluorometh- oxy)phenyl)-6- methyl-3,4- dihydro-2H- pyrano[3,2- b]pyridin-4-amine |
| 67 | 61b | 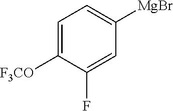 | 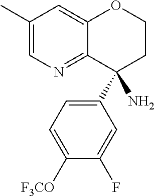 [SMILES: Cc1cnc2c(c1)OCC[C@]2(N)c1ccc(OC(F)(F)F)c(F)c1] | (S)-4-(3-fluoro-4- (trifluorometh- oxy)phenyl)-7- methyl-3,4- dihydro-2H- pyrano[3,2- b]pyridin-4-amine |
| 68 | 61c | 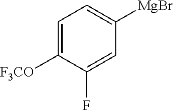 | 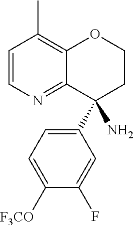 [SMILES: Cc1ccnc2c1OCC[C@]2(N)c1ccc(OC(F)(F)F)c(F)c1] | (S)-4-(3-fluoro-4- (trifluorometh- oxy)phenyl)-8- methyl-3,4- dihydro-2H- pyrano[3,2- b]pyridin-4-amine |
| 69 | 61d | 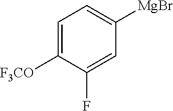 | 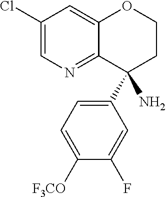 [SMILES: N[C@]1(c2ccc(OC(F)(F)F)c(F)c2)CCOc2cc(Cl)cnc21] | (S)-7-chloro-4- (3-fluoro-4- (trifluorometh- oxy)phenyl)-3,4- dihydro-2H- pyrano[3,2- b]pyridin-4-amine |
| 70 | 61e | 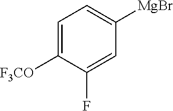 | 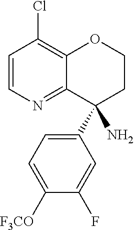 [SMILES: N[C@]1(c2ccc(OC(F)(F)F)c(F)c2)CCOc2c(Cl)ccnc21] | (S)-8-chloro-4- (3-fluoro-4- (Irifluorometh- oxy)phenyl)-3,4- dihydro-2H- pyrano[3,2- b]pyridin-4-amine |
| 71 | 61f | 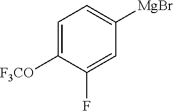 | 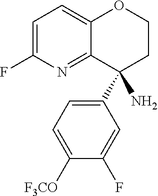 [SMILES: N[C@]1(c2ccc(OC(F)(F)F)c(F)c2)CCOc2ccc(F)nc21] | (S)-6-fluoro-4- (3-fluoro-4- (trifluorometh- oxy)phenyl)-3,4- dihydro-2H- pyrano[3,2- b]pyridin-4-amine |
| 72 | 61g | 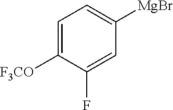 | 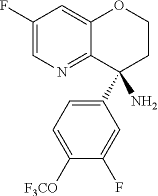 [SMILES: N[C@]1(c2ccc(OC(F)(F)F)c(F)c2)CCOc2cc(F)cnc21] | (S)-7-fluoro-4- (3-fluoro-4- (trifluorometh- oxy)phenyl)-3,4- (dihydro-2H- pyrano[3,2- b]pyridin-4-amine |
| 73 | 61h | 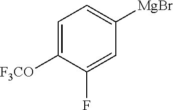 | 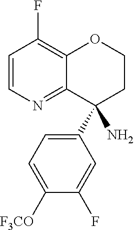 [SMILES: N[C@]1(c2ccc(OC(F)(F)F)c(F)c2)CCOc2c(F)ccnc21] | (S)-8-fluoro-4- (3-fluoro-4- (trifluorometh- oxy)phenyl)-3,4- dihydro-2H- pyrano[3,2- b]pyridin-4-amine |
| 74 | 61i | 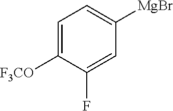 | 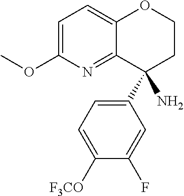 [SMILES: COc1ccc2c(n1)[C@@](N)(c1ccc(OC(F)(F)F)c(F)c1)CCO2] | (S)-4-(3-fluoro-4- (trifluorometh- oxy)phenyl)-6- methoxy-3,4- dihydro-2H- pyrano[3,2- b]pyridin-4-amine |
| 75 | 61j | 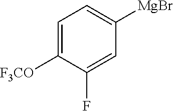 | 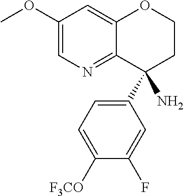 [SMILES: COc1cnc2c(c1)OCC[C@]2(N)c1ccc(OC(F)(F)F)c(F)c1] | (S)-4-(3-fluoro-4- (trifluorometh- oxy)phenyl)-7- methoxy-3,4- dihydro-2H- pyrano[3,2- b]pyridin-4-amine |
| 76 | 61k | 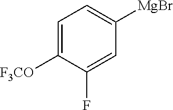 | 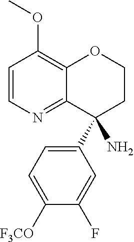 [SMILES: COc1ccnc2c1OCC[C@]2(N)c1ccc(OC(F)(F)F)c(F)c1] | (S)-4-(3-fluoro-4- (trifluorometh- oxy)phenyl)-8- methoxy-3,4- dihydro-2H- pyrano[3,2- b]pyridin-4-amine |
| 77¹ | 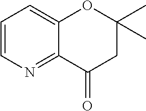 | 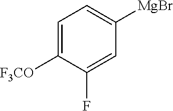 | 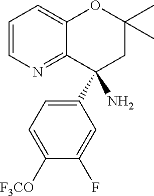 [SMILES: CC1(C)C[C@](N)(c2ccc(OC(F)(F)F)c(F)c2)c2ncccc2O1] | (S)-4-(3-fluoro-4- (trifluorometh- oxy)phenyl)-2,2- dimethyl-3,4- dihydro-2H- pyrano[3,2- b]pyridin-4-amine |
| 78¹ | 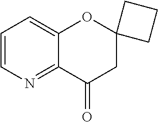 [SMILES: O=C1CC2(CCC2)Oc2cccnc21] | 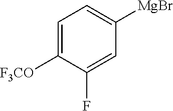 | 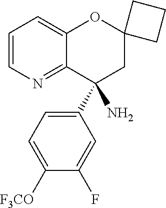 [SMILES: N[C@]1(c2ccc(OC(F)(F)F)c(F)c2)CC2(CCC2)Oc2cccnc21] | (S)-4′-(3-fluoro-4- (trifluorometh- oxy)phenyl)-3′,4′- dihydro- spiro[cyclobutane- 1,2′-pyrano[3,2- b]pyridin]-4′- amine |
| 79 | 52 | 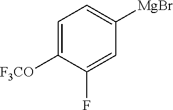 | 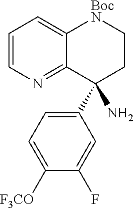 [SMILES: CC(C)(C)OC(=O)N1CC[C@](N)(c2ccc(OC(F)(F)F)c(F)c2)c2ncccc21] | (S)-tert-butyl 4- amino-4-(3- fluoro-4- (trifluorometh- oxy)phenyl)-3,4- dihydro-1,5- naphthyridine- 1(2H)- carboxylate |
| 80 | 53 | 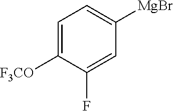 | 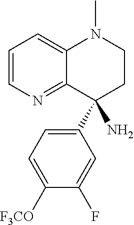 [SMILES: CN1CC[C@](N)(c2ccc(OC(F)(F)F)c(F)c2)c2ncccc21] | (S)-4-(3-fluoro-4- (trifluorometh- oxy)phenyl)-1- methyl-1,2,3,4- tetrahydro-1,5- naphthyridin-4- amine |
| 81 | 54 | 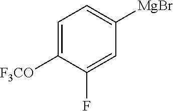 | 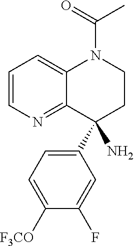 [SMILES: CC(=O)N1CC[C@](N)(c2ccc(OC(F)(F)F)c(F)c2)c2ncccc21] | (S)-1-(4-amino- 4-(3-fluoro-4- (trifluorometh- oxy)phenyl)-3,4- dihydro-1,5- naphthyridin- 1(2H)-yl)ethanone |
| 82 | 55 | 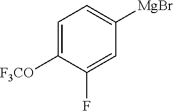 | 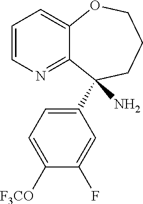 [SMILES: N[C@]1(c2ccc(OC(F)(F)F)c(F)c2)CCCOc2cccnc21] | (S)-9-(3-fluoro-4- (trifluorometh- oxy)phenyl)- 6,7,8,9-tetra- hydrooxepino[3,2- b]pyridin-9-amine |
| 83 | 56 | 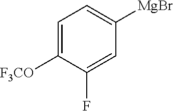 | 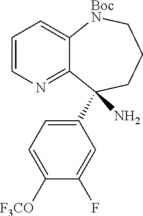 [SMILES: CC(C)(C)OC(=O)N1CCC[C@](N)(c2ccc(OC(F)(F)F)c(F)c2)c2ncccc21] | (S)-tert-butyl 9- amino-9-(3- fluoro-4- (trifluorometh- oxy)phenyl)- 6,7,8,9- tetrahydro-5H- pyrido[3,2- b]azepine-5- carboxylate |
| 84 | 57 | 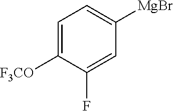 | 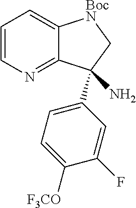 [SMILES: CC(C)(C)OC(=O)N1C[C@](N)(c2ccc(OC(F)(F)F)c(F)c2)c2ncccc21] | (R)-tert-butyl 3- amino-3-(3- fluoro-4- (trifluoro- methoxy)phenyl)- 2,3-dihydro- 1H-pyrrolo[3,2- b]pyridine-1- carboxylate |
| 85² | 1, Step 3 | 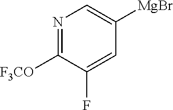 | 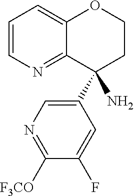 [SMILES: N[C@]1(c2cnc(OC(F)(F)F)c(F)c2)CCOc2cccnc21] | (S)-4-(5-fluoro-6- (trifluorometh- oxy)pyridin-3-yl)- 3,4-dihydro-2H- pyrano[3,2- b]pyridin-4-amine |
| 86³ | 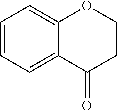 [SMILES: O=C1CCOc2ccccc21] | 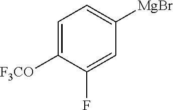 | 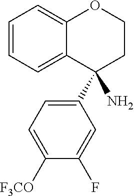 [SMILES: N[C@]1(c2ccc(OC(F)(F)F)c(F)c2)CCOc2ccccc21] | (S)-4-(3-fluoro-4- (trifluorometh- oxy)phenyl)chrom- an-4-amine |
| 87 | 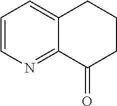 [SMILES: O=C1CCCc2cccnc21] | 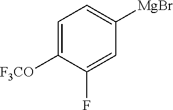 | 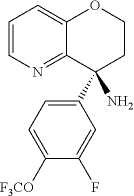 [SMILES: N[C@]1(c2ccc(OC(F)(F)F)c(F)c2)CCOc2cccnc21] | (S)-8-(3-fluoro-4- (trifluorometh- oxy)phenyl)- 5,6,7,8- tetrahydroquinolin- 8-amine |
| 87a | 60b | 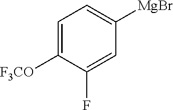 | 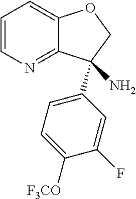 [SMILES: N[C@]1(c2ccc(OC(F)(F)F)c(F)c2)COc2cccnc21] | (S)-3-(3-fluoro-4- (trifluorometh- oxy)phenyl)-2,3- dihydrofuro[3,2- b]pyridin-3-amine |

¹WO2009064418 patent application for ketone intermediate procedures.
²From Intermediate 58.
³Made in an analogous fashion as intermediate 18, followed by chiral separation.

### General Amide Formation Procedure for Examples 338-362

To a solution of intermediate amine hydrochloride, the corresponding carboxylic acid (1.2 eq), and DIPEA (2 eq) in DCM or DMF (1 mL) at room temperature is added an amide coupling reagent such as (HATU, TBTU, or EDCI) (1.2 eq.). The reaction is stirred for 1-24 h at room temperature. The reaction is diluted with DMF (1 mL), filtered through a syringe filter and purified by preperative reverse phase HPLC (gradient elution 10-100% MeCN/0.1% TFA in H₂O). The product containing fractions may then be combined and the solvent removed by lyophilzation to provide the target compound as the TFA salt; or the product is dissolved in MeOH (1 mL) and washed through PL-HCO₃ MP-resin, the resin is further washed with MeOH (2×0.4 mL). The combined filtrates are then concentrated and dried in vacuo to give the title compounds as the free base; or the product containing fractions are concentrated, the solids dissolved in DCM and the organic layer extracted with saturated aqueous NaHCO₃, the organic layer are dried, and concentrated to provide the title compounds as the free base.

TABLE 19
Examples 338-362 can be prepared via amide formation using methods analogous to those described above.

| | Amine | | | |
|---|---|---|---|---|
| | Inter- | | | |
| Ex # | mediate | Acid Structure | Product Structure | Product Name |
| 338 | 62 | 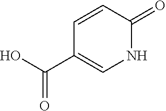 | 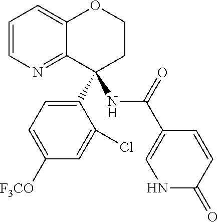 [SMILES: O=C(N[C@]1(c2ccc(OC(F)(F)F)cc2Cl)CCOc2cccnc21)c1ccc(=O)[nH]c1] | (S)-N-(4-(2-chloro- 4-(trifluorometh- oxy)phenyl)-3,4- dihydro-2H- pyrano[3,2-b]pyridin- 4-yl)-6-oxo-1,6- dihydropyridine- 3-carboxamide |
| 339 | 63 | 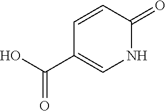 | 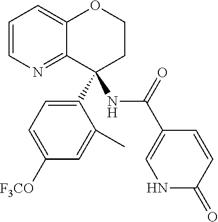 [SMILES: Cc1cc(OC(F)(F)F)ccc1[C@@]1(NC(=O)c2ccc(=O)[nH]c2)CCOc2cccnc21] | (S)-N-(4-(2-methyl- 4-(trifluorometh- oxy)phenyl)-3,4- dihydro-2H- pyrano[3,2-b]pyridin- 4-yl)-6-oxo-1,6- dihydropyridine- 3-carboxamide |
| 340 | 64 | 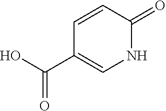 | 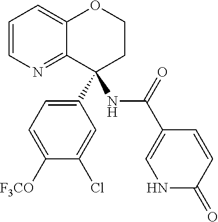 [SMILES: O=C(N[C@]1(c2ccc(OC(F)(F)F)c(Cl)c2)CCOc2cccnc21)c1ccc(=O)[nH]c1] | (S)-N-(4-(3-chloro- 4-(trifluorometh- yl)phenyl)-3,4- dihydro-2H- pyrano[3,2-b]pyridin- 4-yl)-6-oxo-1,6- dihydropyridine- 3-carboxamide |
| 341 | 65 | 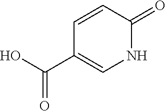 | 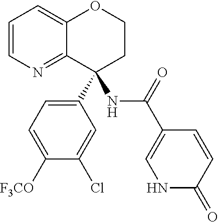 [SMILES: O=C(N[C@]1(c2ccc(OC(F)(F)F)c(Cl)c2)CCOc2cccnc21)c1ccc(=O)[nH]c1] | (S)-N-(4-(3-chloro-4- (trifluorometh- oxy)phenyl)-3,4- dihydro-2H- pyrano[3,2-b]pyridin- 4-yl)-6-oxo-1,6- dihydropyridine- 3-carboxamide |
| 342 | 66 | 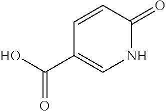 | 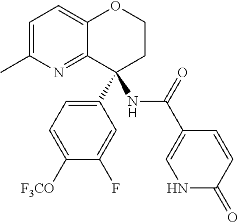 [SMILES: Cc1ccc2c(n1)[C@@](NC(=O)c1ccc(=O)[nH]c1)(c1ccc(OC(F)(F)F)c(F)c1)CCO2] | (S)-N-(4-(3-fluoro-4- (trifluorometh- oxy)phenyl)-6-methyl- 3,4-dihydro-2H- pyrano[3,2-b]pyridin- 4-yl)-6-oxo-1,6- dihydropyridine-3- carboxamide |
| 343 | 67 | 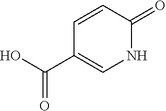 | 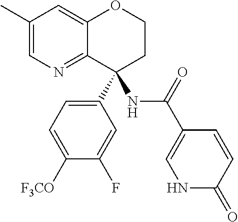 [SMILES: Cc1cnc2c(c1)OCC[C@]2(NC(=O)c1ccc(=O)[nH]c1)c1ccc(OC(F)(F)F)c(F)c1] | (S)-N-(4-(3-fluoro-4- (trifluorometh- oxy)phenyl)-7-methyl- 3,4-dihydro-2H- pyrano[3,2-b]pyridin- 4-yl)-6-oxo-1,6- dihydropyridine-3- carboxamide |
| 344 | 68 | 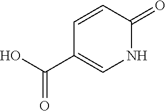 | 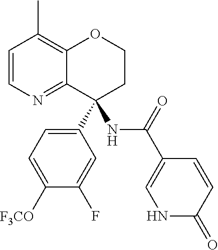 [SMILES: Cc1ccnc2c1OCC[C@]2(NC(=O)c1ccc(=O)[nH]c1)c1ccc(OC(F)(F)F)c(F)c1] | (S)-N-(4-(3-fluoro-4- (trifluorometh- oxy)phenyl)-8-methyl- 3,4-dihydro-2H- pyrano[3,2-b]pyridin- 4-yl)-6-oxo-1,6- dihydropyridine-3- carboxamide |
| 346 | 69 | 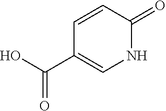 | 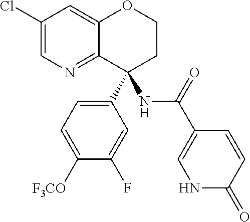 [SMILES: O=C(N[C@]1(c2ccc(OC(F)(F)F)c(F)c2)CCOc2cc(Cl)cnc21)c1ccc(=O)[nH]c1] | (S)-N-(4-(3-fluoro-4- (trifluorometh- oxy)phenyl)-7-chloro- 3,4-dihydro-2H- pyrano[3,2-b]pyridin- 4-yl)-6-oxo-1,6- dihydropyridine-3- carboxamide |
| 347 | 70 | 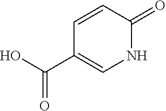 | 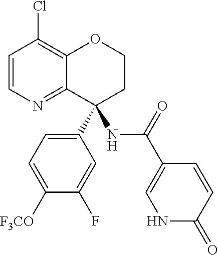 [SMILES: O=C(N[C@]1(c2ccc(OC(F)(F)F)c(F)c2)CCOc2c(Cl)ccnc21)c1ccc(=O)[nH]c1] | (S)-N-(4-(3-fluoro-4- (trifluorometh- oxy)phenyl)-8-chloro- 3,4-dihydro-2H- pyrano[3,2-b]pyridin- 4-yl)-6-oxo-1,6- dihydropyridine-3- carboxamide |
| 348 | 71 | 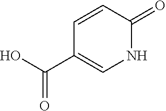 | 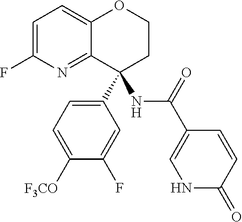 [SMILES: O=C(N[C@]1(c2ccc(OC(F)(F)F)c(F)c2)CCOc2ccc(F)nc21)c1ccc(=O)[nH]c1] | (S)-N-(4-(3-fluoro-4- (trifluorometh- oxy)phenyl)-6-fluoro- 3,4-dihydro-2H- pyrano[3,2-b]pyridin- 4-yl)-6-oxo-1,6- dihydropyridine-3- carboxamide |
| 349 | 72 | 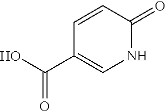 | 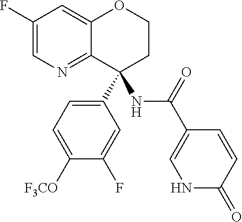 [SMILES: O=C(N[C@]1(c2ccc(OC(F)(F)F)c(F)c2)CCOc2cc(F)cnc21)c1ccc(=O)[nH]c1] | (S)-N-(4-(3-fluoro-4- (trifluorometh- oxy)phenyl)-7-fluoro- 3,4-dihydro-2H- pyrano[3,2-b]pyridin- 4-yl)-6-oxo-1,6- dihydropyridine-3- carboxamide |
| 350 | 73 | 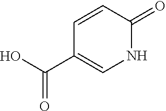 | 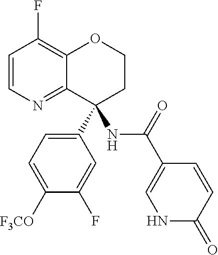 [SMILES: O=C(N[C@]1(c2ccc(OC(F)(F)F)c(F)c2)CCOc2c(F)ccnc21)c1ccc(=O)[nH]c1] | (S)-N-(4-(3-fluoro-4- (trifluorometh- oxy)phenyl)-8-fluoro- 3,4-dihydro-2H- pyrano[3,2-b]pyridin- 4-yl)-6-oxo-1,6- dihydropyridine-3- carboxamide |
| 351 | 74 | 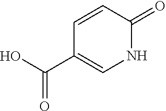 | 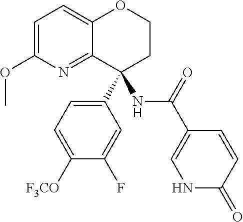 [SMILES: COc1ccc2c(n1)[C@@](NC(=O)c1ccc(=O)[nH]c1)(c1ccc(OC(F)(F)F)c(F)c1)CCO2] | (S)-N-(4-(3-fluoro-4- (trifluorometh- oxy)phenyl)-6-methoxy- 3,4-dihydro-2H- pyrano[3,2-b]pyridin- 4-yl)-6-oxo-1,6- dihydropyridine-3- carboxamide |
| 352 | 75 | 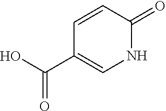 | 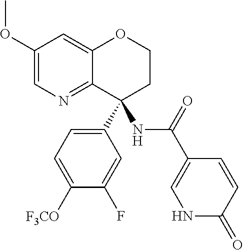 [SMILES: COc1cnc2c(c1)OCC[C@]2(NC(=O)c1ccc(=O)[nH]c1)c1ccc(OC(F)(F)F)c(F)c1] | (S)-N-(4-(3-fluoro-4- (trifluorometh- oxy)phenyl)-7-methoxy- 3,4-dihydro-2H- pyrano[3,2-b]pyridin- 4-yl)-6-oxo-1,6- dihydropyridine-3- carboxamide |
| 353 | 76 | 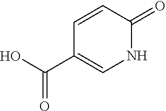 | 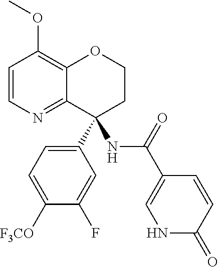 [SMILES: COc1ccnc2c1OCC[C@]2(NC(=O)c1ccc(=O)[nH]c1)c1ccc(OC(F)(F)F)c(F)c1] | (S)-N-(4-(3-fluoro-4- (trifluorometh- oxy)phenyl)-8-methoxy- 3,4-dihydro-2H- pyrano[3,2-b]pyridin- 4-yl)-6-oxo-1,6- dihydropyridine-3- carboxamide |
| 355 | 77 | 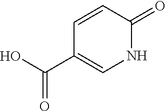 | 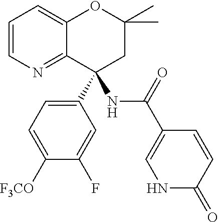 [SMILES: CC1(C)C[C@](NC(=O)c2ccc(=O)[nH]c2)(c2ccc(OC(F)(F)F)c(F)c2)c2ncccc2O1] | (S)-N-(4-(3-fluoro-4- (trifluorometh- oxy)phenyl)-2,2- dimethyl-3,4-dihydro- 2H- pyrano[3,2-b]pyridin- 4-yl)-6-oxo-1,6- dihydropyridine-3- carboxamide |
| 356 | 78 | 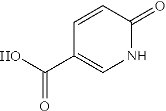 | 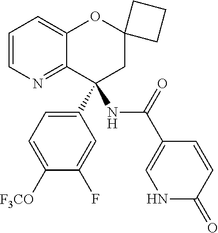 [SMILES: O=C(N[C@]1(c2ccc(OC(F)(F)F)c(F)c2)CC2(CCC2)Oc2cccnc21)c1ccc(=O)[nH]c1] | (S)-N-(4′-(3-fluoro-4- (trifluorometh- oxy)phenyl)-3′,4′- dihydrospiro[cyclo- butane-1,2′- pyrano[3,2-b]pyridin]- 4′-yl)-6-oxo-1,6- dihydropyridine-3- carboxamide |
| 357 | 86 | 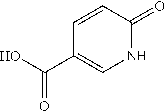 | 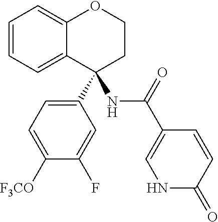 [SMILES: O=C(N[C@]1(c2ccc(OC(F)(F)F)c(F)c2)CCOc2ccccc21)c1ccc(=O)[nH]c1] | (S)-N-(4-(3-fluoro-4- (trifluorometh- oxy)phenyl)chroman- 4-yl)-6-oxo-1,6- dihydropyridine-3- carboxamide |
| 358 | 87 | 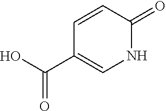 | 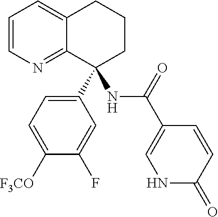 [SMILES: O=C(N[C@]1(c2ccc(OC(F)(F)F)c(F)c2)CCCc2cccnc21)c1ccc(=O)[nH]c1] | (S)-N-(8-(3-fluoro-4- (trifluorometh- oxy)phenyl)-5,6,7,8- tetrahydroquinolin-8- yl)-6-oxo-1,6- dihydropyridine-3- carboxamide |
| 359 | 80 | 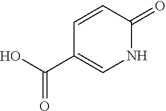 | 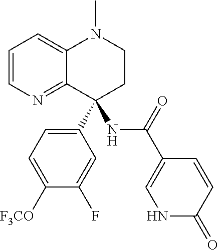 [SMILES: CN1CC[C@](NC(=O)c2ccc(=O)[nH]c2)(c2ccc(OC(F)(F)F)c(F)c2)c2ncccc21] | (S)-N-(4-(3-fluoro-4- (trifluorometh- oxy)phenyl)-1-methyl- 1,2,3,4-tetrahydro-1,5- naphthyridin-4-yl)-6- oxo-1,6- dihydropyridine-3- carboxamide |
| 360 | 81 | 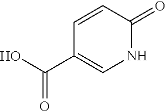 | 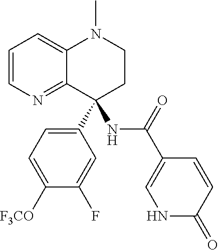 [SMILES: CN1CC[C@](NC(=O)c2ccc(=O)[nH]c2)(c2ccc(OC(F)(F)F)c(F)c2)c2ncccc21] | (S)-N-(1-acetyl-4-(3- fluoro-4-(trifluorometh- oxy)phenyl)-1,2,3,4- tetrahydro-1,5- naphthyridin-4-yl)-6- oxo-1,6- dihydropyridine-3- carboxamide |
| 361 | 82 | 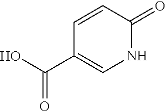 | 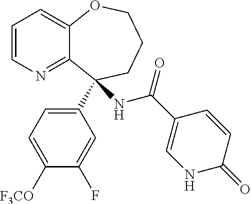 [SMILES: O=C(N[C@]1(c2ccc(OC(F)(F)F)c(F)c2)CCCOc2cccnc21)c1ccc(=O)[nH]c1] | (S)-N-(9-(3-fluoro-4- (trifluorometh- oxy)phenyl)-6,7,8,9- tetrahydrooxe- pino[3,2-b]pyridin- 9-yl)-6-oxo-1,6- dihydropyridine-3- carboxamide |
| 362 | 85 | 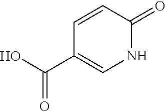 | 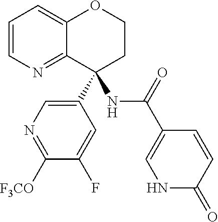 [SMILES: O=C(N[C@]1(c2cnc(OC(F)(F)F)c(F)c2)CCOc2cccnc21)c1ccc(=O)[nH]c1] | (S)-N-(4-(5-fluoro-6- (trifluorometh- oxy)pyridin-3-yl)-3,4- dihydro-2H- pyrano[3,2-b]pyridin- 4-yl)-6-oxo-1,6- dihydropyridine-3- carboxamide |
| 362a | 60a | 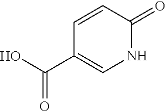 | 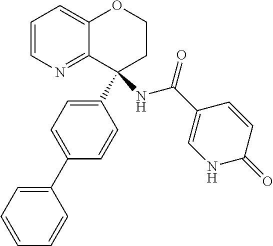 [SMILES: O=C(N[C@]1(c2ccc(-c3ccccc3)cc2)CCOc2cccnc21)c1ccc(=O)[nH]c1] | (S)-N-(4-([1,1′- biphenyl]-4-yl)-3,4- dihydro-2H- pyrano[3,2-b]pyridin- 4-yl)-6-oxo-1,6- dihydropyridine-3- carboxamide |
| 362b | 87a | 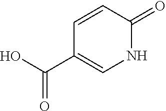 | 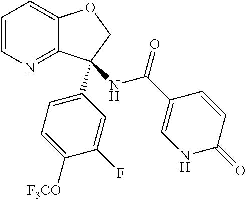 [SMILES: O=C(N[C@]1(c2ccc(OC(F)(F)F)c(F)c2)COc2cccnc21)c1ccc(=O)[nH]c1] | (S)-N-(3-(3-fluoro-4- (trifluorometh- oxy)phenyl)-2,3-di- hydrofuro[3,2-b]pyridin- 3-yl)-6-oxo-1,6- dihydropyridine-3- carboxamide |

The amide coupling may also be followed by an additional step. For example, hydrolysis of an ester is exemplified in Example 75 and may be performed after the amide coupling to provide the compounds in Table 20.

TABLE 20
Examples 363-366 can be prepared via amide formation using methods analogous to those described above followed
by ester hydrolysis.

| | Amine | | | |
|---|---|---|---|---|
| | Inter- | | | |
| Ex # | mediate | Acid Structure | Product Structure | Product Name |
| 363 | 64 | 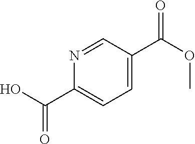 | 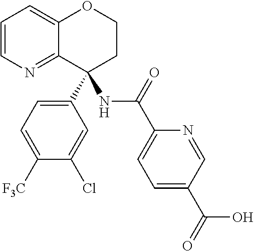 [SMILES: O=C(O)c1ccc(C(=O)N[C@]2(c3ccc(C(F)(F)F)c(Cl)c3)CCOc3cccnc32)nc1] | (S)-6-((4-(3-chloro-4- (trifluoromethyl)phenyl)- 3,4-dihydro-2H- pyrano[3,2-b]pyridin-4- yl)carbamoyl)nicotinic acid |
| 364 | 65 | 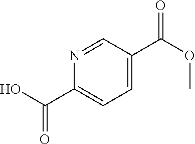 | 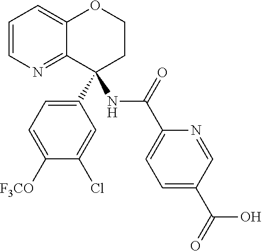 [SMILES: O=C(O)c1ccc(C(=O)N[C@]2(c3ccc(OC(F)(F)F)c(Cl)c3)CCOc3cccnc32)nc1] | (S)-6-((4-(3-chloro-4- (trifluoromethoxy)phenyl)- 3,4-dihydro-2H- pyrano[3,2-b]pyridin-4- yl)carbamoyl)nicotinic acid |
| 365 | 86 | 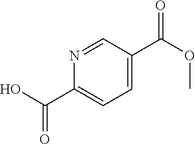 | 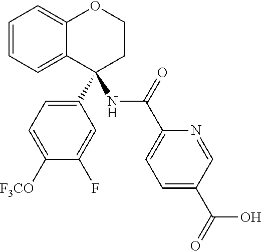 [SMILES: O=C(O)c1ccc(C(=O)N[C@]2(c3ccc(OC(F)(F)F)c(F)c3)CCOc3ccccc32)nc1] | (S)-6-((4-(3-fluoro-4- (trifluoromethoxy)phen- yl)chroman-4- yl)carbamoyl)nicotinic acid |
| 366 | 87 | 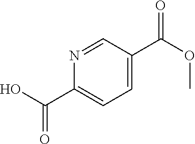 | 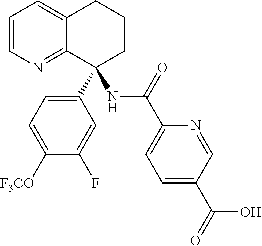 [SMILES: O=C(O)c1ccc(C(=O)N[C@]2(c3ccc(OC(F)(F)F)c(F)c3)CCCc3cccnc32)nc1] | (S)-6-((8-(3-fluoro-4- (trifluoromethoxy)phenyl)- 5,6,7,8-tetrahydroquinolin- 8- yl)carbamoyl)nicotinic acid |

Deprotection of a Boc protecting group may be performed after the amide coupling to provide the compounds in Table 21. The compounds may be synthesized according to the general procedure amide coupling procedure described above, followed by deprotection.

### General Procedure for Boc Deprotection

To a solution of Boc protected material (1 eq) and DCM (0.2 M) is added 4M HCl in dioxane (5 eq.). After 1-24 h, the reaction can be washed with water and saturated NaHCO₃. The organic layer may then be concentrated in vacuo and purified by column chromatography to give the title compound.

TABLE 21
Examples 367-369 can be prepared via amide formation using methods analogous to those above followed by Boc deprotection.

| | Amine | | | |
|---|---|---|---|---|
| | Inter- | | | |
| Ex # | mediate | Acid Structure | Product Structure | Product Name |
| 367 | 79 | 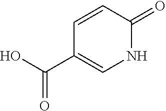 | 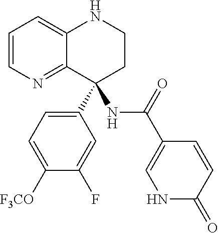 [SMILES: O=C(N[C@]1(c2ccc(OC(F)(F)F)c(F)c2)CCNc2cccnc21)c1ccc(=O)[nH]c1] | (S)-N-(4-(3-fluoro-4- (trifluoromethoxy)phenyl)- 1,2,3,4-tetrahydro-1,5- naphthyridin-4-yl)-6-oxo- 1,6-dihydropyridine-3- carboxamide |
| 368 | 83 | 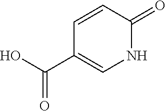 | 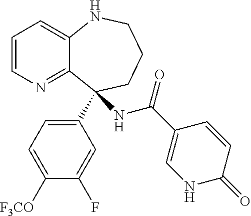 [SMILES: O=C(N[C@]1(c2ccc(OC(F)(F)F)c(F)c2)CCCNc2cccnc21)c1ccc(=O)[nH]c1] | (S)-N-(9-(3-fluoro-4- (trifluoromethoxy)phenyl)- 6,7,8,9-tetrahydro-5H- pyrido[3,2-b]azepin-9-yl)- 6-oxo-1,6- dihydropyridine-3- carboxamide |
| 369 | 84 | 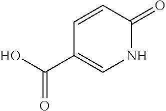 | 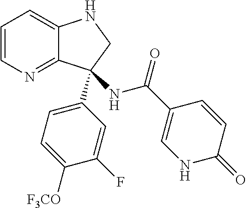 [SMILES: O=C(N[C@]1(c2ccc(OC(F)(F)F)c(F)c2)CNc2cccnc21)c1ccc(=O)[nH]c1] | (R)-N-(3-(3-fluoro-4- (trifluoromethoxy)phenyl)- 2,3-dihydro-1H- pyrrolo[3,2-b]pyridin- 3-yl)-6-oxo-1,6- dihydropyridine-3- carboxamide |

### Stereochemistry

Absolute stereochemistry for Example 2, was determined by comparison of VCD spectra (VCD, Biotools, Inc.) (Stephens, P. J. et. al, Chirality 2008, 20, 643). Comparison of experimental VCD data with ab initio DFT calculations provide for the assigned absolute stereochemistry.

### Assays

### Luminescence Readout Assay for Measuring Intracellular Calcium

A stable Chinese hamster ovary cell line expressing human TRPM8 was generated using tetracycline inducible T-REx™ expression system from Invitrogen, Inc. (Carlsbad, Calif.). In order to enable a luminescence readout based on intracellular increase in calcium (Le Poul et al., 2002), the cell line was also co-transfected with pcDNA3.1 plasmid containing jelly fish aequorin cDNA. Twenty four hours before the assay, cells were seeded in 96-well plates and TRPM8 expression was induced with 0.5 μg/ml tetracycline. On the day of the assay, culture media was removed and cells were incubated with assay buffer

(Ham's F12 containing 30 mM HEPES) that contained 15 μM coelenterazine (P.J.K, Germany) for 2 h. Potential antagonists were added 2.5 min prior to the addition of agonist, 1 μM icilin, 100 μM L-menthol, or 1 min prior to the addition of cold buffer (<10° C.). The luminescence was measured by a CCD camera based FLASH-luminometer built by Amgen, Inc. A cooling device attached to FLASH luminometer was used for cold activation. Compound activity was calculated using either GraphPad Prism 4.01 (GraphPad Software Inc, San Diego, Calif.) or Genedata Screener.

The following compounds exhibit IC₅₀ values of less than 5 μM in the assay described above with icilin activation. Results are shown in Table 22. The prophetic examples may be tested using the same procedure and will be found to inhibit TRPM8.

TABLE 22
hTRPM8 IC₅₀'s for Examples 1-337.

| Example | IC₅₀ (μM) |
|---|---|
| 1 | 0.014 |
| 2 | 0.086 |
| 3 | 0.025 |
| 4 | 0.009 |
| 5 | 0.021 |
| 6 | 0.034 |
| 7 | 0.032 |
| 8 | 0.075 |
| 9 | 0.038 |
| 10 | 0.163 |
| 11 | 2.57 |
| 12 | 2.07 |
| 13 | 0.883 |
| 14 | 0.091 |
| 15 | 0.054 |
| 16 | 0.357 |
| 17 | 0.060 |
| 18 | 0.162 |
| 19 | 0.012 |
| 20 | 0.010 |
| 21 | 0.007 |
| 22 | 0.014 |
| 23 | 0.012 |
| 24 | 0.023 |
| 25 | 0.019 |
| 26 | 0.014 |
| 27 | 0.046 |
| 28 | 1.66 |
| 29 | 0.143 |
| 30 | 0.019 |
| 31 | 0.784 |
| 32 | 0.051 |
| 33 | 0.005 |
| 34 | 0.024 |
| 35 | 0.2 |
| 36 | 0.036 |
| 37 | 2.62 |
| 38 | 0.020 |
| 39 | 0.269 |
| 40 | 0.007 |
| 41 | 0.087 |
| 42 | 0.024 |
| 43 | 0.111 |
| 44 | 0.008 |
| 45 | 0.038 |
| 46 | 0.358 |
| 47 | 0.082 |
| 48 | 0.481 |
| 49 | 0.043 |
| 50 | 0.008 |
| 51 | 0.005 |
| 52 | 0.016 |
| 53 | 0.015 |
| 54 | 0.011 |
| 55 | 0.022 |
| 56 | 0.021 |
| 57 | 0.004 |
| 58 | 0.021 |
| 59 | 0.013 |
| 60 | 0.125 |
| 61 | 0.006 |
| 62 | 0.051 |
| 63 | 0.019 |
| 64 | 0.335 |
| 65 | 0.012 |
| 66 | 0.017 |
| 67 | 0.293 |
| 68 | 0.023 |
| 69 | 0.036 |
| 70 | 1.35 |
| 71 | 0.143 |
| 72 | 0.112 |
| 73 | 0.019 |
| 74 | 0.025 |
| 75 | 0.014 |
| 76 | 0.206 |
| 77 | 1.34 |
| 78 | 0.058 |
| 79 | 0.334 |
| 80 | 0.021 |
| 81 | 0.124 |
| 82 | 0.134 |
| 83 | 0.028 |
| 84 | 0.266 |
| 85 | 0.012 |
| 86 | 0.019 |
| 87 | 0.024 |
| 88 | 0.115 |
| 89 | 0.058 |
| 90 | 0.036 |
| 91 | 0.019 |
| 92 | 0.054 |
| 93 | 0.022 |
| 94 | 0.230 |
| 95 | 0.094 |
| 96 | 0.006 |
| 97 | 0.159 |
| 98 | 0.023 |
| 99 | 0.106 |
| 100 | 0.187 |
| 101 | 0.760 |
| 102 | 0.218 |
| 103 | 0.278 |
| 104 | 0.085 |
| 105 | 0.404 |
| 106 | 0.039 |
| 107 | 0.150 |
| 108 | 0.386 |
| 109 | 3.06 |
| 110 | 0.585 |
| 111 | 1.52 |
| 112 | 0.233 |
| 113 | 0.053 |
| 114 | 0.05 |
| 115 | 0.341 |
| 116 | 0.284 |
| 117 | 0.0737 |
| 118 | 2.17 |
| 119 | 0.879 |
| 120 | 0.524 |
| 121 | 1.25 |
| 122 | 1.52 |
| 123 | 3.56 |
| 124 | 1.41 |
| 125 | 0.429 |
| 126 | 0.908 |
| 127 | 0.336 |
| 128 | 0.501 |
| 129 | 2.6 |
| 130 | 1 |
| 131 | 0.113 |
| 132 | 0.216 |
| 133 | 0.156 |
| 134 | 0.320 |
| 135 | 0.113 |
| 136 | 0.784 |
| 137 | 1.52 |
| 138 | 3.16 |
| 139 | 0.122 |
| 140 | 0.340 |
| 141 | 0.260 |
| 142 | 0.336 |
| 143 | 0.176 |
| 144 | 1.92 |
| 145 | 0.070 |
| 146 | 0.088 |
| 147 | 1.53 |
| 148 | 0.770 |
| 149 | 0.299 |
| 150 | 1.19 |
| 151 | 1.64 |
| 152 | 2.40 |
| 153 | 0.112 |
| 154 | 0.091 |
| 155 | 0.016 |
| 156 | 0.008 |
| 157 | 0.012 |
| 158 | 0.006 |
| 159 | 0.046 |
| 160 | 0.039 |
| 161 | 0.042 |
| 162 | 0.092 |
| 163 | 0.102 |
| 164 | 2.23 |
| 165 | 0.531 |
| 166 | 0.010 |
| 167 | 0.019 |
| 168 | 0.146 |
| 169 | 0.032 |
| 170 | 0.021 |
| 171 | 0.012 |
| 172 | 0.022 |
| 173 | 0.475 |
| 174 | 0.128 |
| 175 | 0.043 |
| 176 | 0.054 |
| 177 | 0.209 |
| 178 | 0.072 |
| 179 | 0.305 |
| 180 | 0.032 |
| 181 | 0.014 |
| 182 | 0.036 |
| 183 | 0.021 |
| 184 | 0.050 |
| 185 | 0.089 |
| 186 | 0.066 |
| 187 | 0.054 |
| 188 | 0.015 |
| 189 | 0.134 |
| 190 | 0.152 |
| 191 | 0.011 |
| 192 | 0.094 |
| 193 | 0.584 |
| 194 | 0.044 |
| 195 | 0.006 |
| 196 | 0.007 |
| 197 | 0.092 |
| 198 | 0.035 |
| 199 | 0.012 |
| 200 | 0.163 |
| 201 | 0.021 |
| 202 | 0.027 |
| 203 | 0.074 |
| 204 | 0.007 |
| 205 | 0.004 |
| 206 | 0.013 |
| 207 | 0.001 |
| 208 | 0.011 |
| 209 | 0.045 |
| 210 | 2.31 |
| 211 | 0.336 |
| 212 | 1.69 |
| 213 | 0.081 |
| 214 | 0.190 |
| 215 | 0.011 |
| 216 | 0.270 |
| 217 | 0.046 |
| 218 | 0.010 |
| 219 | 0.011 |
| 220 | 0.016 |
| 221 | 0.031 |
| 222 | 2.00 |
| 223 | 0.010 |
| 224 | 0.116 |
| 225 | 0.012 |
| 226 | 0.150 |
| 227 | 0.0587 |
| 228 | 0.105 |
| 229 | 0.145 |
| 230 | 0.0201 |
| 231 | 0.0452 |
| 232 | 0.0258 |
| 233 | 0.222 |
| 234 | 0.0568 |
| 235 | 0.0357 |
| 236 | 0.0665 |
| 237 | 0.00407 |
| 238 | 0.0414 |
| 239 | 0.0346 |
| 240 | 0.0406 |
| 241 | 0.769 |
| 242 | 0.00555 |
| 243 | 0.0166 |
| 244 | 0.0521 |
| 245 | 0.254 |
| 246 | 0.0944 |
| 247 | 0.030 |
| 248 | 0.024 |
| 249 | 0.099 |
| 250 | 0.015 |
| 251 | 0.056 |
| 252 | 0.024 |
| 253 | 0.004 |
| 254 | 0.025 |
| 255 | 0.009 |
| 256 | 0.048 |
| 257 | 0.059 |
| 258 | 0.034 |
| 259 | 0.088 |
| 260 | 0.032 |
| 261 | 0.032 |
| 262 | 0.007 |
| 263 | 0.095 |
| 264 | 0.074 |
| 265 | 0.014 |
| 266 | 0.036 |
| 267 | 0.037 |
| 268 | 0.071 |
| 269 | 0.024 |
| 270 | 0.044 |
| 271 | 0.016 |
| 272 | 0.183 |
| 273 | 0.019 |
| 274 | 0.189 |
| 275 | 0.034 |
| 276 | 0.016 |
| 277 | 0.085 |
| 278 | 0.068 |
| 279 | 0.053 |
| 280 | 0.026 |
| 281 | 0.051 |
| 282 | 0.007 |
| 283 | 0.044 |
| 284 | 0.006 |
| 285 | 0.179 |
| 286 | 0.754 |
| 287 | 0.017 |
| 288 | 0.045 |
| 289 | 0.018 |
| 290 | 0.086 |
| 291 | 0.015 |
| 292 | 0.014 |
| 293 | 0.007 |
| 294 | 0.003 |
| 295 | 0.015 |
| 296 | 0.008 |
| 297 | 0.026 |
| 298 | 0.023 |
| 299 | 0.115 |
| 300 | 0.012 |
| 301 | 0.266 |
| 302 | 0.004 |
| 303 | 0.028 |
| 304 | 0.028 |
| 305 | 0.015 |
| 306 | 0.159 |
| 307 | 0.631 |
| 308 | 0.011 |
| 309 | 0.055 |
| 310 | 0.008 |
| 311 | 0.155 |
| 312 | 0.104 |
| 313 | 0.014 |
| 314 | 0.004 |
| 315 | 0.834 |
| 316 | 0.089 |
| 317 | 3.34 |
| 318 | 0.025 |
| 319 | 0.015 |
| 320 | 3.70 |
| 321 | 0.012 |
| 322 | 0.009 |
| 323 | 0.316 |
| 324 | 0.103 |
| 325 | 0.118 |
| 326 | 0.033 |
| 327 | 0.023 |
| 328 | 1.56 |
| 329 | 0.231 |
| 330 | 0.094 |
| 331 | 0.034 |
| 332 | 0.016 |
| 333 | 0.025 |
| 334 | 1.01 |
| 335 | 0.051 |
| 336 | 0.086 |
| 337 | 0.070 |

### Icilin Biochemical Challenge Models

### Inhibition of Icilin Induced Jumping in Mice

Example compounds at doses ranging from 0.01 to 10 mg/kg may be administered to male C57BL/6 mice (18-25 g, Taconic, n=10/treatment) 1 h before icilin to assess the ability to block the spontaneous jumps induced by icilin (i.p. suspended in 100% PEG400 at 20 mg/kg, 5 mL/kg). The total number of jumps will be recorded during the 10 min post-icilin administration based on the number of photocell beam breaks from the vertical array of open field boxes (Kinder Scientific) while movement of the mice will be restricted within a clear Plexiglas® cylinder 9.5 cm diameter×30 cm height.

### Inhibition of Icilin Induced Shaking in Rats

Example compounds at doses ranging from 0.01 to 3 mg/kg (p.o, suspended in 5% Tween80/Oralplus or suspended in 2% HPMC-1% Tween-80 pH2.2 with MSA, 5 mL/kg) can be administered to male Sprague Dawley rats (200-300 g, Harlan, n=6-8/treatment) 2 h before icilin to assess the ability to block the spontaneous wet-dog shake phenomena induced by icilin (i.p., suspended in 100% PEG400 at 0.5 mg/kg, 1 mL/kg or p.o., suspended in 2% HPMC-1% Tween-80 at 3 mg/kg, 2.5 mL/kg). Spontaneous wet-dog shakes may be counted manually by two blinded observers or using LABORAS automation (Metris) for 30 min post-icilin. The synthesized and prophetic example compounds may be measured using these procedures and will be found to reduce the spontaneous wet dog shake phenomena induced by icilin.

TABLE 23
Percent Inhibition of WetDog Shakes in rat following the
indicated dose.

| | | % Inhibition | | | % Inhibition |
|---|---|---|---|---|---|
| | Dose | of Wet-Dog | | Dose | of Wet-Dog |
| Example # | (mg/kg) | Shakes | Example | (mg/kg) | Shakes |
| 21 | 1 | 98 | 283 | 1 | 78 |
| 50 | 1 | 67 | 289 | 1 | 93 |
| 58 | 1 | 97 | 291 | 1 | 97 |
| 59 | 1 | 99 | 310 | 1 | 98 |
| 61 | 1 | 80 | 313 | 1 | 99 |
| 75 | 1 | 99 | 314 | 1 | 99 |
| 83 | 1 | 59 | 318 | 1 | 86 |
| 85 | 1 | 72 | 319 | 0.3 | 75 |
| 155 | 1 | 60 | 321 | 1 | 95 |
| 158 | 1 | 92 | 326 | 1 | 98 |
| 170 | 1 | 90 | 327 | 1 | 93 |
| 204 | 1 | 98 | 332 | 1 | 99 |

### Cold Pressor Test (CPT) as a Translatable PD Model for TRPM8

The cold pressor test (CPT) was developed as a method to measure blood pressure response following exposure to a cold stimulus and has been used over 70 years in the diagnosis of hypertension and other cardiac autonomic disorders (Hines and Brown 1936). In healthy human subjects, the CPT is typically performed by immersing a subject's hand into ice water (0-5° C.) which triggers, through a vascular sympathetic activation of afferent pain and temperature neurons, an increase in blood pressure. With some modifications, this test has also been utilized in rat to delineate the medullary and spinal pathways mediating the cardio-vascular responses to cold pressor test and to identify neurotransmitters in these pathways (Sapru N et al 2008) or to characterize analgesic compounds (Koltzenburg M et al 2006 and Player M R et al 2011).

TRPM8 antagonists may be evaluated in rat CPT to determine whether they will attenuate the increase in blood pressure resulting from exposure to cold stimulation of the paws and ventral half of the body. Male Sprague-Dawley rats weighing 350-450 g may be instrumented with a unilateral carotid artery-cannula connected to a transducer for measuring blood pressure using a Digi-Med Blood Pressure Analyzer, Model 400 Animals may then be orally administrated with Vehicle (2% HPMC 1% Tween 80 pH 2.2 with MSA) or test compounds at 120 min prior to cold challenge and anesthetized with sodium pentobarbital at 60 mg/kg ip at 100 min prior to cold. Blood pressure may be recorded for 5 min for pre-cold baseline and additional 5 min during immersion of the paws and ventral half of body in ice water. Percent inhibition attributed to treatment with test compound may then be determined using the following Formula: [1-(cold evoked change in MBP/cold evoked change in MBP post-vehicle)]×100. Plasma may be collected through artery catheter immediately after CPT for pk analysis and IC_{50/90} determination.

### REFERENCES

Hines, E A and Brown G E. The cold pressor test for measuring the reactability of the blood pressure. Am. Heart J. 1936, 11:1-9
Nakamura T, Kawabe K, and Sapru H. Cold pressor test in the rat: medullary and spinal pathways and neurotransmitters. Am J Physiol Heart Circ Physiol 2008, 295:H1780-H1787
Koltzenburg M, Pokorny R, Gasser U and Richarz U. Differential sensitivity of three experimental pain models in detecting the analgesic effects of transdermal fentanyl and buprenorphine. Pain 2006, 126:165-174
Parks D, Parsons W, Colburn R, Meegala S, Ballentine S, Illig C, Qin N, Liu Y, Hutchinson T, Lubin M, Stone D, Baker J, Schneider C, Ma J, Damiano B, Flores C, and Player M. Design and optimization of benzimidazole-containing transient receptor potentiate melastatin 8 (TRPM8) antagonists. J. Med. Chem. 2011, 54:233-247

### CCI Model

Surgery—A chronic constriction injury (CCl) can be produced as previously described (Bennett & Xie, 1988). Under gaseous anesthesia with a mixture of isoflurane (3% for induction and 2% for maintenance) in O₂, the sciatic nerve can be exposed at the mid-thigh level proximal to the sciatic trifurcation. Four chromic gut ligatures (4-0) can be tied loosely around nerve, 1-2 mm apart such that the vascular supply will not be compromised.

Behavioral testing—A behavioral test can be performed to estimate cold-induced ongoing pain as previously described (Choi et al., 1994). The rat can be placed under a transparent plastic cover on an aluminum plate (IITC PE34, Woodland, Calif.) which can be kept at a cold temperature (5±0.5° C.). After 2 min of adaptation, the cumulative duration of time that the rat lifts its foot off the plate for the next 5 min can be measured. Foot lifts associated with locomotion or grooming are not counted. Seven to 9 days after the CCl surgery, baseline of the cold-induced ongoing pain can be measured. Any rat showing a cold-induced ongoing pain less than 100 sec out of 300 sec observation period can be eliminated from the study. Twenty four hours after the baseline measurement, test compound, positive control, morphine (2 mg/kg, Sigma, St. Louis) or a vehicle (saline or 2% HPMC/1% Tween 80) can be administered orally (test compound) or subcutaneously (morphine). Two hours (test compound) or 30 mins (morphine) after the drug administration, the cold-induced ongoing pain can be measured again.

### Chung Model

Surgery—Spinal nerve ligation surgery can be performed as previously described (Kim & Chung, 1992). Briefly, under gaseous anesthesia with a mixture of isoflurane (3% for induction and 2% for maintenance) in O₂, the spinal nerve injury can be produced by ligating the left L5 and L6 spinal nerves taking special care to avoid any possible damage to the L4 spinal nerve or surrounding area. Additional treatments can be performed to increase the development of mechanical allodynia. First, L5 spinal nerve can be cut approximately 1 mm distal to the suture as described by Li et al. (2000). Second, immediately after ligation and cut, the L4 spinal nerve can be lightly manipulated by slightly stretching it with a fine hooked glass rod and gently sliding the hook back and forth 20 times along the nerve as described by Lee et al. (2003). The whole surgery procedure from anesthesia to the clipping of the incised skin can take at most 15 min.

Behavioral testing—Two weeks later, mechanical sensitivity can be measured by determining the median 50% foot withdrawal threshold for von Frey filaments using the up-down method (Chaplan et al., 1994). The rats can be placed under a plastic cover (9×9×20 cm) on a metal mesh floor. The area tested consists of the middle glabrous area between the footpads of the plantar surface of the hind paw. The plantar area can be touched with a series of 9 von Frey hairs with approximately exponentially incremental bending forces (von Frey values: 3.61, 3.8, 4.0, 4.2, 4.41, 4.6, 4.8, 5.0 and 5.2; equivalent to: 0.41, 0.63, 1.0, 1.58, 2.51, 4.07, 6.31, 10 and 15.8 g). The von Frey hair can be presented perpendicular to the plantar surface with sufficient force to cause slight bending, and held for approximately 3-4 sec. Abrupt withdrawal of the foot (paw flinching, shaking or licking for more than 1 sec) can be recorded as a response. Any rat showing a mechanical threshold of more than 3.16 g or less than 0.7 g after surgery can be eliminated from the study. After measuring basal threshold, test compound, positive control gabapentin (Sigma, St. Louis) or a vehicle (saline or 2% HPMC/1% Tween 80) can be administered orally (test compound) or intraperitoneally (gabapentin). The measurement of the tactile threshold can be reassessed at 1.5 and 2 h after drug administration.

Data—Since the von Frey filament set is calibrated on a logarithmic scale by the vendor (Stoelting) and our selection of 9 filaments for the up-down method is also based on near equal logarithmic intervals (Dixon et al., 1980), data can be treated using logarithmic values in every aspect (statistical treatment as well as plotting). However, an equivalent gram value scale is labeled on the Y-axis of the figures for convenience. Data are expressed as mean±standard error of the mean (S.E.M.).

For the treatment of TRPM8-receptor-diseases, such as acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders, the compounds of the present invention may be administered orally, parentally, by inhalation spray, rectally, or topically in dosage unit formulations containing conventional pharmaceutically acceptable carriers, adjuvants, and vehicles. The term parenteral as used herein includes, subcutaneous, intravenous, intramuscular, intrasternal, infusion techniques or intraperitoneally.

Treatment of diseases and disorders herein is intended to also include the prophylactic administration of a compound of the invention, a pharmaceutical salt thereof, or a pharmaceutical composition of either to a subject (i.e., an animal, preferably a mammal, most preferably a human) believed to be in need of preventative treatment, such as, for example, pain, inflammation and the like.

The dosage regimen for treating TRPM8-receptor-mediated diseases, cancer, and/or hyperglycemia with the compounds of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods. Dosage levels of the order from about 0.01 mg to 30 mg per kilogram of body weight per day, preferably from about 0.1 mg to 10 mg/kg, more preferably from about 0.25 mg to 1 mg/kg are useful for all methods of use disclosed herein.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a capsule, a tablet, a suspension, or liquid. The pharmaceutical composition is preferably made in the form of a dosage unit containing a given amount of the active ingredient. For example, these may contain an amount of active ingredient from about 1 to 2000 mg, preferably from about 1 to 500 mg, more preferably from about 5 to 150 mg. A suitable daily dose for a human or other mammal may vary widely depending on the condition of the patient and other factors, but, once again, can be determined using routine methods.

The active ingredient may also be administered by injection as a composition with suitable carriers including saline, dextrose, or water. The daily parenteral dosage regimen will be from about 0.1 to about 30 mg/kg of total body weight, preferably from about 0.1 to about 10 mg/kg, and more preferably from about 0.25 mg to 1 mg/kg.

Injectable preparations, such as sterile injectable aqueous or oleaginous suspensions, may be formulated according to the known are using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

Formulations suitable for topical administration include liquid or semi-liquid preparations suitable for penetration through the skin (e.g., liniments, lotions, ointments, creams, or pastes) and drops suitable for administration to the eye, ear, or nose.

For administration, the compounds of this invention are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulfuric acids, acacia, gelatin, sodium alginate, polyvinyl-pyrrolidine, and/or polyvinyl alcohol, and tableted or encapsulated for conventional administration. Alternatively, the compounds of this invention may be dissolved in saline, water, polyethylene glycol, propylene glycol, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting, sweetening, flavoring, and perfuming agents.

Compounds of the present invention can possess one or more asymmetric carbon atoms and are thus capable of existing in the form of optical isomers as well as in the form of racemic or non-racemic mixtures thereof. The optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid and then separation of the mixture of diastereoisomers by crystallization followed by liberation of the optically active bases from these salts. A different process for separation of optical isomers involves the use of a chiral chromatography column optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound. The optically active compounds of the invention can likewise be obtained by using active starting materials. These isomers may be in the form of a free acid, a free base, an ester or a salt.

Likewise, the compounds of this invention may exist as isomers, that is compounds of the same molecular formula but in which the atoms, relative to one another, are arranged differently. In particular, the alkylene substituents of the compounds of this invention, are normally and preferably arranged and inserted into the molecules as indicated in the definitions for each of these groups, being read from left to right. However, in certain cases, one skilled in the art will appreciate that it is possible to prepare compounds of this invention in which these substituents are reversed in orientation relative to the other atoms in the molecule. That is, the substituent to be inserted may be the same as that noted above except that it is inserted into the molecule in the reverse orientation. One skilled in the art will appreciate that these isomeric forms of the compounds of this invention are to be construed as encompassed within the scope of the present invention.

The compounds of the present invention can be used in the form of salts derived from inorganic or organic acids. The salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methansulfonate, nicotinate, 2-naphthalenesulfonate, oxalate, palmoate, pectinate, persulfate, 2-phenylpropionate, picrate, pivalate, propionate, succinate, tartrate, thiocyanate, tosylate, mesylate, and undecanoate. Also, the basic nitrogen-containing groups can be quaternized with such agents as lower alkyl halides, such as methyl, ethyl, propyl, and butyl chloride, bromides and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids that may be employed to from pharmaceutically acceptable acid addition salts include such inorganic acids as hydrochloric acid, sulfuric acid and phosphoric acid and such organic acids as oxalic acid, maleic acid, succinic acid and citric acid. Other examples include salts with alkali metals or alkaline earth metals, such as sodium, potassium, calcium or magnesium or with organic bases.

Also encompassed in the scope of the present invention are pharmaceutically acceptable esters of a carboxylic acid or hydroxyl containing group, including a metabolically labile ester or a prodrug form of a compound of this invention. A metabolically labile ester is one which may produce, for example, an increase in blood levels and prolong the efficacy of the corresponding non-esterified form of the compound. A prodrug form is one which is not in an active form of the molecule as administered but which becomes therapeutically active after some in vivo activity or biotransformation, such as metabolism, for example, enzymatic or hydrolytic cleavage. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use. Esters of a compound of this invention, may include, for example, the methyl, ethyl, propyl, and butyl esters, as well as other suitable esters formed between an acidic moiety and a hydroxyl containing moiety. Metabolically labile esters, may include, for example, methoxymethyl, ethoxymethyl, iso-propoxymethyl, α-methoxyethyl, groups such as α-((C₁-C₄)alkyloxy)ethyl, for example, methoxyethyl, ethoxyethyl, propoxyethyl, iso-propoxyethyl, etc.; 2-oxo-1,3-dioxolen-4-ylmethyl groups, such as 5-methyl-2-oxo-1,3,dioxolen-4-ylmethyl, etc.; C₁-C₃ alkylthiomethyl groups, for example, methylthiomethyl, ethylthiomethyl, isopropylthiomethyl, etc.; acyloxymethyl groups, for example, pivaloyloxymethyl, α-acetoxymethyl, etc.; ethoxycarbonyl-1-methyl; or α-acyloxy-α-substituted methyl groups, for example α-acetoxyethyl.

Further, the compounds of the invention may exist as crystalline solids which can be crystallized from common solvents such as ethanol, N,N-dimethyl-formamide, water, or the like. Thus, crystalline forms of the compounds of the invention may exist as polymorphs, solvates and/or hydrates of the parent compounds or their pharmaceutically acceptable salts. All of such forms likewise are to be construed as falling within the scope of the invention.

While the compounds of the invention can be administered as the sole active pharmaceutical agent, they can also be used in combination with one or more compounds of the invention or other agents. When administered as a combination, the therapeutic agents can be formulated as separate compositions that are given at the same time or different times, or the therapeutic agents can be given as a single composition.

The foregoing is merely illustrative of the invention and is not intended to limit the invention to the disclosed compounds. Variations and changes which are obvious to one skilled in the art are intended to be within the scope and nature of the invention which are defined in the appended claims.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of Formula I having the structure: 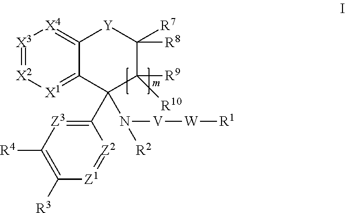
a pharmaceutically-acceptable salt thereof, a tautomer thereof, a pharmaceutically-acceptable salt of the tautomer, a stereoisomer thereof, or a mixture thereof, wherein:
V is selected from —C(═O)— or —S(═O)₂—;
W is absent or is selected from —NH—, —NR^{1a}—, or O;
X¹ is selected from —CR⁵— or —N—;
X² is selected from —CR⁵— or —N—;
X³ is selected from —CR⁵— or —N—;
X⁴ is selected from —CR⁵— or —N—;
Y is selected from —O—, —CH₂—, —NH—, —NR^{1b}—, —CF₂—, —C(═O)—, —C(H)(F)—, or —C(H)(OH)—;
Z¹ is selected from —CR⁶— or —N—;
Z² is selected from —CR⁶— or —N—;
Z³ is selected from —CR⁶— or —N—;
wherein 0, 1, or 2 of X¹, X², X³, and X⁴ are N;
wherein 0, 1, or 2 of Z¹, Z², and Z³ are N;
m is 0, 1, or 2;
R¹ is C₁₋₆alk or a direct-bonded, C₁₋₂alk-linked, C₁₋₂alkO-linked, saturated, partially-saturated or unsaturated 3-, 4-, 5-, 6- or 7-membered monocyclic or 7-, 8-, 9-, 10- or 11-membered bicyclic ring containing 0, 1, 2, 3 or 4 heteroatoms selected from N, O and S, but containing no more than one O or S atom, the C₁₋₆alk and ring being substituted by 0, 1, 2 or 3 substituents independently selected from halo, oxo, C₁₋₆alk, C₁₋₆alkOH, C₁₋₆alkOH substituted by 1, 2, or 3 halo substituents, C₁₋₆alk-C(═O)R^{a}, C₁₋₆alk-C(═O)OR^{a}, C₁₋₄haloalk, cyano, nitro, —C(═O)R^{a}, —C(═O)OR^{a}, —C(═O)NR^{a}R^{a}, —C(═O)NR^{a}S(═O)₂R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, ═S, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the ring is additionally substituted by 0 or 1 directly bonded, SO₂ linked, C(═O) linked or CH₂ linked saturated, partially-saturated or unsaturated 3-, 4-, 5-, 6- or 7-membered monocyclic ring containing 0, 1, 2, 3 or 4 heteroatoms selected from N, O and S, but containing no more than one O or S atom, and substituted by 0, 1, 2 or 3 groups selected from halo, oxo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —SR^{a}, —S(═O)R^{a}, —S(═O)₂R^{a}, —S(═O)₂NR^{a}R^{a}, —NR^{a}R^{a}, and —N(R^{a})C(═O)R^{a};
R^{1a} is C₁₋₆alk;
R^{1b} is C₁₋₆alk or —C(═O)R^{b};
R² is H or C₁₋₆alk;
R³ is H, C₁₋₈alk, C₁₋₈alkOH, C₁₋₄haloalk, halo, cyano, R^{b}, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} or —NR^{a}C₂₋₆alkOR^{a};
R⁴ is H, C₁₋₆alk, —C₁₋₃haloalk, —OC₁₋₆alk, —OC₁₋₃haloalk, —N(C₁₋₆alk)C₁₋₆alk, —NHC₁₋₆alk, —NC(═O)C₁₋₆alk, —N(C₁₋₆alk)C₁₋₆alk, F, Cl, Br, CN, OH or NH₂; or R³ and R⁴ together form a four-atom unsaturated bridge containing 0 or 1 N atoms, wherein the bridge is substituted by 0, 1 or 2 R⁵ substituents;
R⁵ is, at each instance, independently selected from H, C₁₋₈alk, C₁₋₈alkOH, C₁₋₄haloalk, halo, cyano, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} or —NR^{a}C₂₋₆alkOR^{a};
R⁶ is, at each instance, independently selected from H, halo, ORE, C₁₋₆alk, or CF₃;
R⁷ and R⁸ are independently selected from H or C₁₋₆alk, or R⁷ and R⁸, together with the carbon atom to which they are attached, join to form a 3 to 7 membered cycloalkyl ring or a 3-7 membered heterocyclyl ring that includes 1 or 2 heteroatoms selected from O, N, or S;
R⁹ and R¹⁶ are, at each instance, independently selected from H or C₁₋₆alk;
R^{a} is independently, at each instance, H or R^{b}; and
R^{b} is independently, at each instance, phenyl, benzyl or C₁₋₆alk, and when R³ is R^{b}, R^{b} may additionally be an unsaturated 5 or 6-membered monocyclic ring containing 1, 2, or 3 heteroatoms selected from N, O, and S, the phenyl, benzyl C₁₋₆alk, and unsaturated 5 or 6-membered monocyclic ring being substituted by 0, 1, 2 or 3 substituents selected from halo, cyano, oxo, C₁₋₄alk, C₁₋₄alkOH, C₁₋₃haloalk, —OC₁₋₄alk, —OH, —NH₂, —OC₁₋₄alk, —OC₁₋₄haloalk, —S(═O)₂C₁₋₄alk, —NHC(═O)—C₁₋₄alk, —C(═O)NH₂, —C(═O)NHC₁₋₄alk, —C(═O)N(C₁₋₄alk)₂, —NHC₁₋₄alk, and —N(C₁₋₄alk)C₁₋₄alk, or a saturated, partially-saturated, or unsaturated 5 or 6-membered monocyclic ring containing 1 or 2 heteroatoms selected from N, O, and S;
wherein the compound is not one of the following compounds and is not a salt thereof: 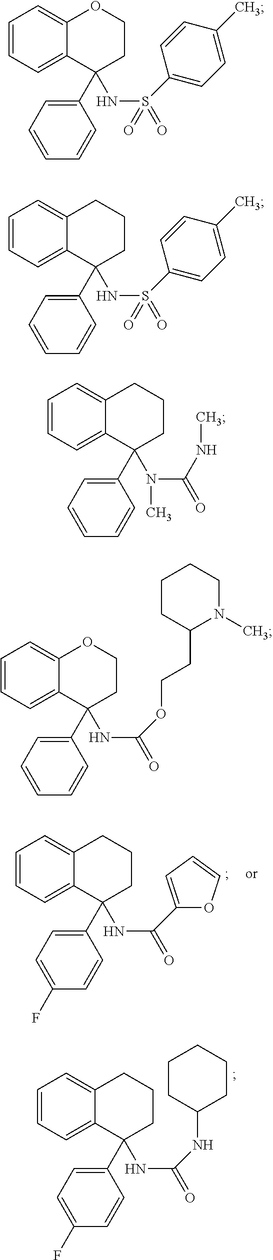
and further wherein, the compound is not one of the following compounds, is not a salt thereof, is not a tautomer thereof, is not a salt of a tautomer, is not a stereoisomer thereof, and is not a salt of a stereoisomer:

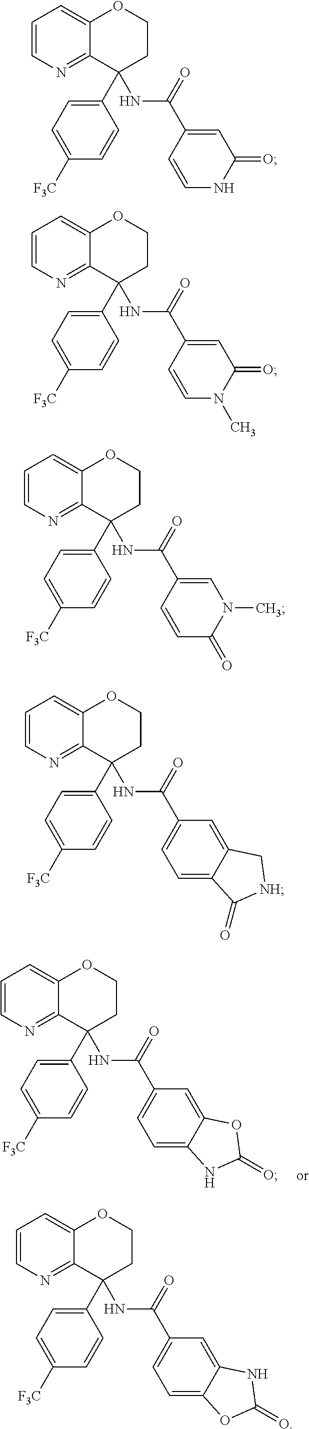

2. The compound of claim 1 or the pharmaceutically-acceptable salt thereof, the tautomer thereof, the pharmaceutically-acceptable salt of the tautomer, the stereoisomer thereof, or the mixture thereof, wherein R⁷ and R⁸ are selected from H and —CH₃.

3. The compound of claim 1 or the pharmaceutically-acceptable salt thereof, the tautomer thereof, the pharmaceutically-acceptable salt of the tautomer, the stereoisomer thereof, or the mixture thereof, wherein m is 1.

4. The compound of claim 1 or the pharmaceutically-acceptable salt thereof, the tautomer thereof, the pharmaceutically-acceptable salt of the tautomer, the stereoisomer thereof, or the mixture thereof, wherein Y is —O—.

5. The compound of claim 1 or the pharmaceutically-acceptable salt thereof, the tautomer thereof, the pharmaceutically-acceptable salt of the tautomer, the stereoisomer thereof, or the mixture thereof, wherein R⁹ and R¹⁰ are H.

6. The compound of claim 1 or the pharmaceutically-acceptable salt thereof, the tautomer thereof, the pharmaceutically-acceptable salt of the tautomer, the stereoisomer thereof, or the mixture thereof, wherein the compound of Formula I has the Formula II: 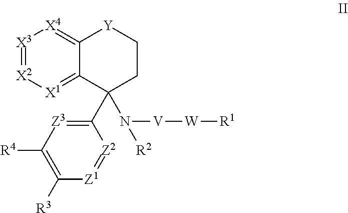
wherein:
Y is selected from —O— or —CH₂—.

7. The compound of claim 6 or the pharmaceutically-acceptable salt thereof, the tautomer thereof, the pharmaceutically-acceptable salt of the tautomer, the stereoisomer thereof, or the mixture thereof, wherein the compound of Formula II has the Formula IIA:

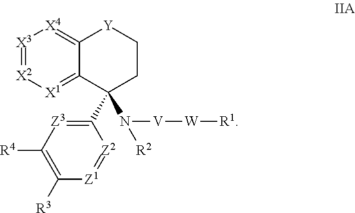

8. The compound of claim 7 or the pharmaceutically-acceptable salt thereof, the tautomer thereof, the pharmaceutically-acceptable salt of the tautomer, the stereoisomer thereof, or the mixture thereof, wherein 1 of X¹, X², X³, and X⁴ is N.

9. The compound of claim 7 or the pharmaceutically-acceptable salt thereof, the tautomer thereof, the pharmaceutically-acceptable salt of the tautomer, the stereoisomer thereof, or the mixture thereof, wherein X¹ is N and X², X³, and X⁴ are all —CR⁵—.

10. The compound of claim 8 or the pharmaceutically-acceptable salt thereof, the tautomer thereof, the pharmaceutically-acceptable salt of the tautomer, the stereoisomer thereof, or the mixture thereof, wherein 0 or 1 of Z¹, Z², and Z³ are N.

11. The compound of claim 10 or the pharmaceutically-acceptable salt thereof, the tautomer thereof, the pharmaceutically-acceptable salt of the tautomer, the stereoisomer thereof, or the mixture thereof, wherein 0 of Z¹, Z², and Z³ are N.

12. The compound of claim 1 or the pharmaceutically-acceptable salt thereof, the tautomer thereof, the pharmaceutically-acceptable salt of the tautomer, the stereoisomer thereof, or the mixture thereof, wherein V is —C(═O)—.

13. The compound of claim 12 or the pharmaceutically-acceptable salt thereof, the tautomer thereof, the pharmaceutically-acceptable salt of the tautomer, the stereoisomer thereof, or the mixture thereof, wherein W is absent.

14. The compound of claim 3 or the pharmaceutically-acceptable salt thereof, the tautomer thereof, the pharmaceutically-acceptable salt of the tautomer, the stereoisomer thereof, or the mixture thereof, wherein R² is H.

15. The compound of claim 1 or the pharmaceutically-acceptable salt thereof, the tautomer thereof, the pharmaceutically-acceptable salt of the tautomer, the stereoisomer thereof, or the mixture thereof, wherein the compound of Formula I has the Formula III 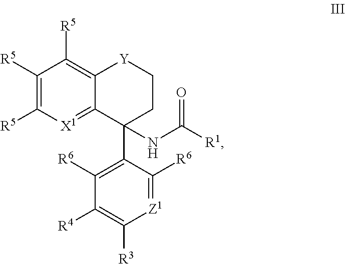
wherein Y is —O— or —CH₂—.

16. The compound of claim 1 or the pharmaceutically-acceptable salt thereof, the tautomer thereof, the pharmaceutically-acceptable salt of the tautomer, the stereoisomer thereof, or the mixture thereof, wherein R³ is selected from —CH₃, —F, —Cl, —CF₃, or —OCF₃.

17. The compound of claim 16 or the pharmaceutically-acceptable salt thereof, the tautomer thereof, the pharmaceutically-acceptable salt of the tautomer, the stereoisomer thereof, or the mixture thereof, wherein R³ is selected from —CF₃ or —OCF₃.

18. The compound of claim 1 or the pharmaceutically-acceptable salt thereof, the tautomer thereof, the pharmaceutically-acceptable salt of the tautomer, the stereoisomer thereof, or the mixture thereof, wherein R³ is R^{b} and R^{b} is a phenyl substituted by 0, 1, 2 or 3 substituents selected from halo, cyano, oxo, C₁₋₄alk, C₁₋₄alkOH, C₁₋₃haloalk, —OC₁₋₄alk, —OH, —NH₂, —OC₁₋₄alk, —OC₁₋₄haloalk, —S(═O)₂C₁₋₄alk, —NHC(═O)—C₁₋₄alk, —C(═O)NH₂, —C(═O)NHC₁₋₄alk, —C(═O)N(C_{1 -4}-alk)₂, —NHC₁₋₄alk, and —N(C₁₋₄alk)C₁₋₄alk, or a saturated, partially-saturated, or unsaturated 5 or 6-membered monocyclic ring containing 1 or 2 heteroatoms selected from N, O, and S.

19. The compound of claim 1 or the pharmaceutically-acceptable salt thereof, the tautomer thereof, the pharmaceutically-acceptable salt of the tautomer, the stereoisomer thereof, or the mixture thereof, wherein R³ is R^{b} and R^{b} is an unsaturated 5 or 6-membered monocyclic ring containing 1, 2, or 3 heteroatoms selected from N, O, and S, wherein the 5 or 6-membered monocyclic ring is substituted by 0. 1, 2, or 3 substituents selected from halo, cyano, oxo, C₁₋₄alk, C₁₋₄alkOH, C₁₋₃haloalk, —OC₁₋₄alk, —OH, —NH₂, —OC₁₋₄alk, —OC₁₋₄haloalk, —S(═O)₂Cl₁₋₄alk, —NHC(═O)—C₁₋₄alk, —C(═O)NH₂, —C(═O)NHC₁₋₄alk, —C(═O)N(C_{1 -4}-alk)₂, —NHC₁₋₄alk, and —N(C₁₋₄alk)C₁₋₄alk, or a saturated, partially-saturated, or unsaturated 5 or 6-membered monocyclic ring containing 1 or 2 heteroatoms selected from N, O, and S.

20. The compound of claim 1 or the pharmaceutically-acceptable salt thereof, the tautomer thereof, the pharmaceutically-acceptable salt of the tautomer, the stereoisomer thereof, or the mixture thereof, wherein R³ is R^{b} and R^{b} is a pyridyl, pyrazinyl, pyrimidinyl, isoxazolyl, or furanyl substituted by 0. 1, 2, or 3 substituents selected from halo, cyano, oxo, C₁₋₄alk, C₁₋₄alkOH, C₁₋₃haloalk, —OC₁₋₄alk, —OH, —NH₂, —OC₁₋₄alk, —OC₁₋₄haloalk, —S(═O)₂C₁₋₄alk, —NHC(═O)—C₁₋₄alk, —C(═O)NH₂, —C(═O)NHC₁₋₄alk, —C(═O)N(C₁₋₄alk)₂, —NHC₁₋₄alk, and —N(C₁₋₄alk)C₁₋₄alk, or a saturated, partially-saturated, or unsaturated 5 or 6-membered monocyclic ring containing 1 or 2 heteroatoms selected from N, O, and S.

21. The compound of claim 1 or the pharmaceutically-acceptable salt thereof, the tautomer thereof, the pharmaceutically-acceptable salt of the tautomer, the stereoisomer thereof, or the mixture thereof, wherein R⁴ is H.

22. The compound of claim 1 or the pharmaceutically-acceptable salt thereof, the tautomer thereof, the pharmaceutically-acceptable salt of the tautomer, the stereoisomer thereof, or the mixture thereof, wherein R⁴ is selected from F, Cl, C₁₋₆alk, —OC_{1-b}alk, —OC₁₋₃haloalk, or —C₁₋₃haloalk.

23. The compound of claim 1 or the pharmaceutically-acceptable salt thereof, the tautomer thereof, the pharmaceutically-acceptable salt of the tautomer, the stereoisomer thereof, or the mixture thereof, wherein R⁴ is —F.

24. The compound of claim 1 or the pharmaceutically-acceptable salt thereof, the tautomer thereof, the pharmaceutically-acceptable salt of the tautomer, the stereoisomer thereof, or the mixture thereof, wherein —R³ is —OCF₃ and R⁴ is —F.

25. The compound of claim 1 or the pharmaceutically-acceptable salt thereof, the tautomer thereof, the pharmaceutically-acceptable salt of the tautomer, the stereoisomer thereof, or the mixture thereof, wherein each instance of R⁵ is H.

26. The compound of claim 1 or the pharmaceutically-acceptable salt thereof, the tautomer thereof, the pharmaceutically-acceptable salt of the tautomer, the stereoisomer thereof, or the mixture thereof, wherein in at least one instance, R⁵ is selected from C₁₋₈alk, halo, or —OR^{a}.

27. The compound of claim 1 or the pharmaceutically-acceptable salt thereof, the tautomer thereof, the pharmaceutically-acceptable salt of the tautomer, the stereoisomer thereof, or the mixture thereof, wherein in at least one instance, R⁵ is selected from —CH₃, —Cl, —F, or —OMe.

28. The compound of claim 1 or the pharmaceutically-acceptable salt thereof, the tautomer thereof, the pharmaceutically-acceptable salt of the tautomer, the stereoisomer thereof, or the mixture thereof, wherein each instance of R⁶ is H.

29. The compound of claim 1 or the pharmaceutically-acceptable salt thereof, the tautomer thereof, the pharmaceutically-acceptable salt of the tautomer, the stereoisomer thereof, or the mixture thereof, wherein at least one instance of R⁶ is halo or C₁₋₆alk.

30. The compound of claim 1 or the pharmaceutically-acceptable salt thereof, the tautomer thereof, the pharmaceutically-acceptable salt of the tautomer, the stereoisomer thereof, or the mixture thereof, wherein R¹ is the saturated, partially-saturated or unsaturated 3-, 4-, 5-, 6- or 7-membered monocyclic or 7-, 8-, 9-, 10- or 11-membered bicyclic ring and the monocyclic or bicyclic ring is substituted by 0, 1, 2, or 3 substituents, wherein the substituents are selected from F, Cl, Br, I, oxo, cyano, —CH₃, —CH₂CH₃, —CH₂CH₂CH₃, —C(H)(CH₃)₂, —CH₂C(H)(CH₃)₂, —CH₂C(H)═CH₂, —CH₂CO₂H, —CH₂CF₃, —C(OH)(CH₃)₂, —SO₂N(H)CH₃, —N(H)SO₂CH₃, —OCH₃, —OCF₃, —OH, —OCH₂CO₂H, —CH₂OH, —CH₂CH₂OH, —CH₂C(H)(CH₃)OH, —CO₂H, —CO₂CH₃, —CO₂CH₂CH₃, —CO₂C(CH₃)₃, —CO₂NH₂, —CO₂N(H)CH₃, —SO₂CH₃, —OC(═O)CH₃, —NH₂, —NHC(═O)CH₃, —N(CH₃)₂, —N(H)CH₂CH₃, —CF₃, —CHF₂, —CH₂C(H)(CF₃)OH, —CH₂C(CH₃)₂OH, —CH₂-phenyl, —C(═O)-phenyl, tetrazolyl, oxadiazolonyl, pyridyl, oxetanyl,

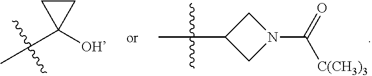 [SMILES: *C1(O)CC1 ; *C1CN(C(=O)C(C)(C)C)C1]

31. The compound of claim 1 or the pharmaceutically-acceptable salt thereof, the tautomer thereof, the pharmaceutically-acceptable salt of the tautomer, the stereoisomer thereof, or the mixture thereof, wherein R¹ is a phenyl, pyridyl, pyridinonyl, piperidinonyl, pyridazinonyl, pyrazinonyl, pyridazinyl, pyrimidinyl, pyrazinyl, tetradyrofuranyl, tetrahydropyranyl, thiazolyl, isothiazolyl, furanyl, thiophenyl, pyrazolyl, isoxazolyl, triazolyl, oxazolyl, imidazolyl, pyrrolidinonyl, piperidinyl, cyclohexyl, cyclohexanonyl, quinolinyl, isoquinolinyl, naphthyridinyl, pyrrolopyridinyl, pyrrolopyrimidinyl, benzothiophenyl, pyrazolopyrimidinyl, triazolopyrimidinyl, indazolyl, tetrahydroindazolyl, tetrahydrocyclopentapyrazolyl, dihydropyrazolooxazinyl, indolinonyl, isoindolinonyl, benzooxazolonyl, oxazolopyridinonyl, benzoimidazolonyl, isoindolindionyl, tetrahydroquinolinyl, dihydroquinolinonyl, benzooxazinonyl, dihydrobenzooxazinonyl, dihydroindenonyl, benzothiazolyl, benzimidazolyl, imidazopyridinyl, tetrazolopyridinyl, quinolinonyl, quinoxalinyl, indolyl, or quinoxalindionyl substituted by 0, 1, 2, or 3 substituents.

32. The compound of claim 1 or the pharmaceutically-acceptable salt thereof, the tautomer thereof, the pharmaceutically-acceptable salt of the tautomer, the stereoisomer thereof, or the mixture thereof, wherein R¹ is a phenyl, pyridyl, pyridinonyl, pyrazinonyl, pyridazinyl, pyrimidinyl, tetrahydropyranyl, thiazolyl, isothiazolyl, imidazolyl, piperidinyl, quinolinyl, isoquinolinyl, indazolyl, indolinonyl, isoindolinonyl, benzooxazolonyl, dihydroquinolinonyl, imidazopyridinyl, quinolinonyl, indolyl substituted by 0, 1, 2, or 3 substituents.

33. The compound of claim 32 or the pharmaceutically-acceptable salt thereof, the tautomer thereof, the pharmaceutically-acceptable salt of the tautomer, the stereoisomer thereof, or the mixture thereof, wherein R¹ is a phenyl substituted by 0, or 1 substituent.

34. The compound of claim 32 or the pharmaceutically-acceptable salt thereof, the tautomer thereof, the pharmaceutically-acceptable salt of the tautomer, the stereoisomer thereof, or the mixture thereof, wherein R¹ is a pyridinonyl substituted by 0, or 1 substituent.

35. The compound of claim 32 or the pharmaceutically-acceptable salt thereof, the tautomer thereof, the pharmaceutically-acceptable salt of the tautomer, the stereoisomer thereof, or the mixture thereof, wherein R¹ is a pyridyl substituted by 0, or 1 substituent.

36. The compound of claim 32 or the pharmaceutically-acceptable salt thereof, the tautomer thereof, the pharmaceutically-acceptable salt of the tautomer, the stereoisomer thereof, or the mixture thereof, wherein R¹ is a benzooxazolonyl substituted by 0, or 1 substituent.

37. The compound of claim 32 or the pharmaceutically-acceptable salt thereof, the tautomer thereof, the pharmaceutically-acceptable salt of the tautomer, the stereoisomer thereof, or the mixture thereof, wherein R¹ is a quinolinyl substituted by 0, or 1 substituent.

38. The compound of claim 1 or the pharmaceutically-acceptable salt thereof, the tautomer thereof, the pharmaceutically-acceptable salt of the tautomer, the stereoisomer thereof, or the mixture thereof, wherein R¹ is a group of formula 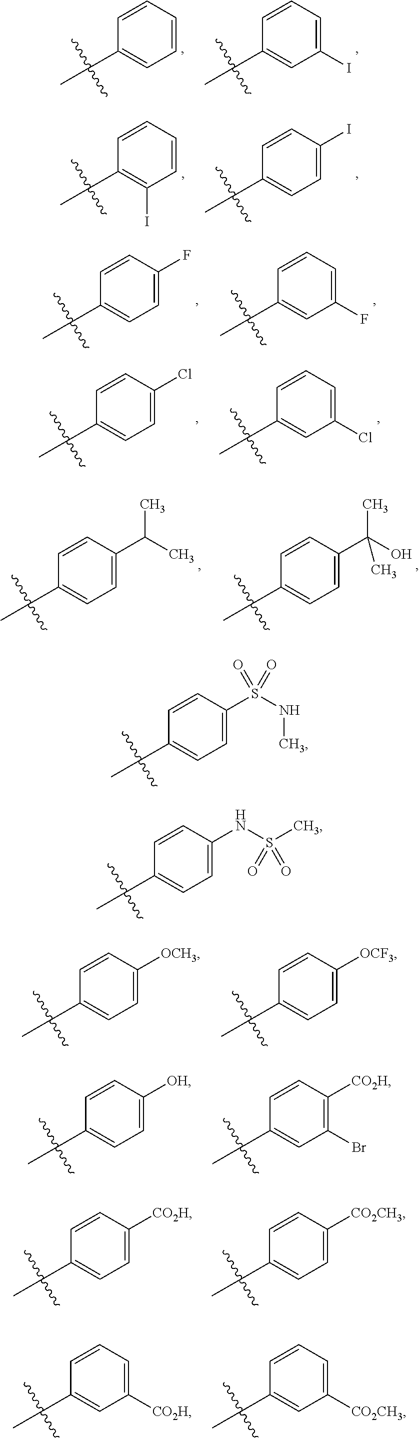 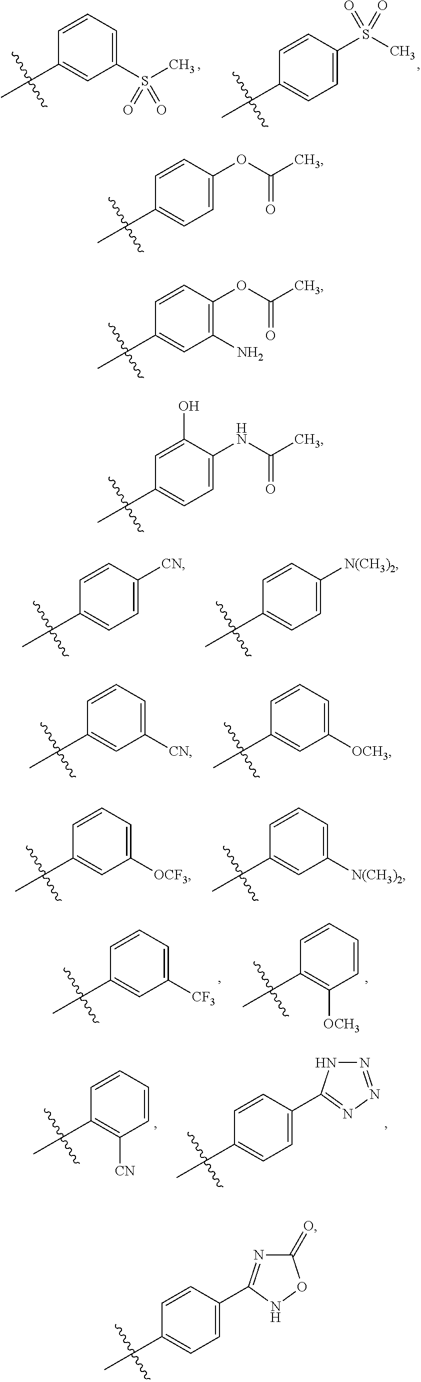 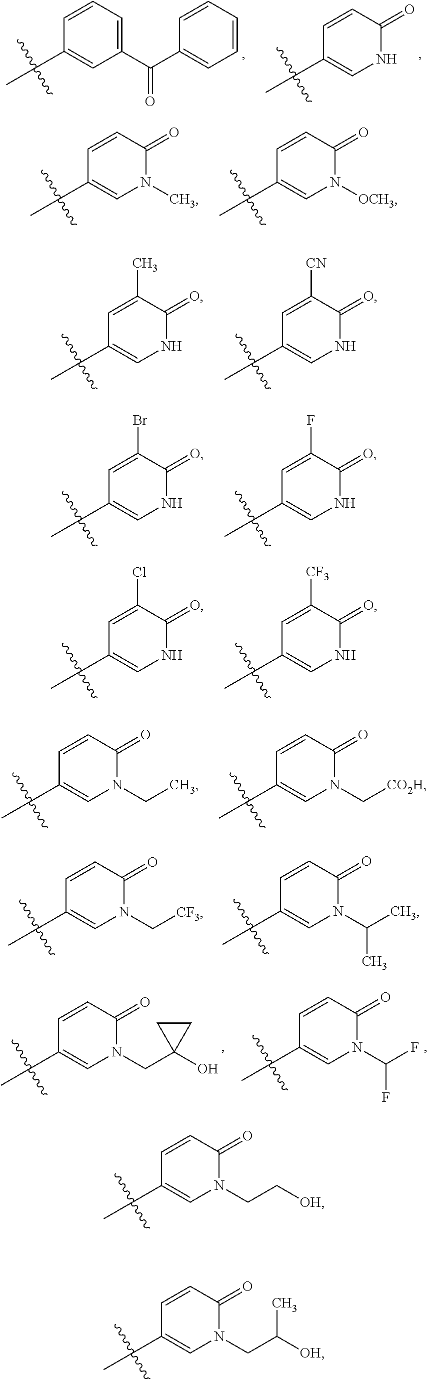 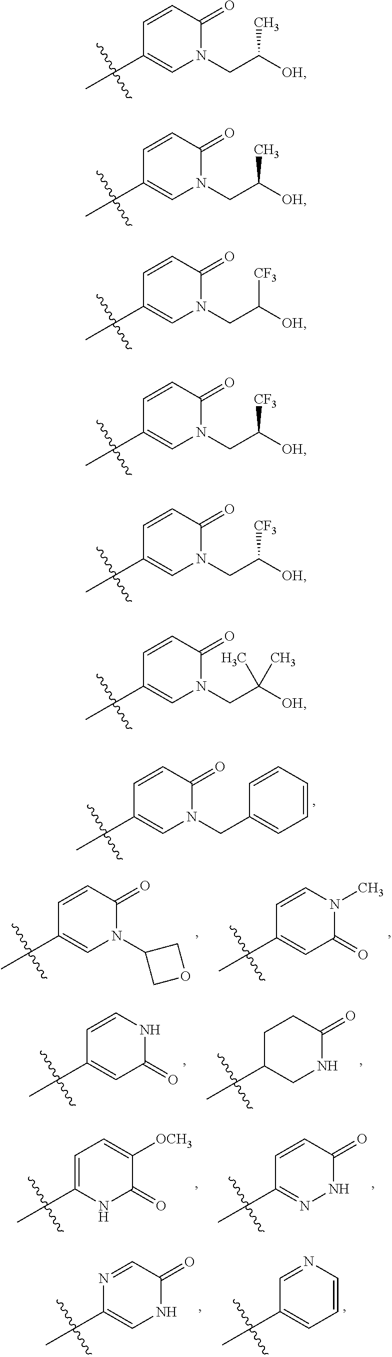 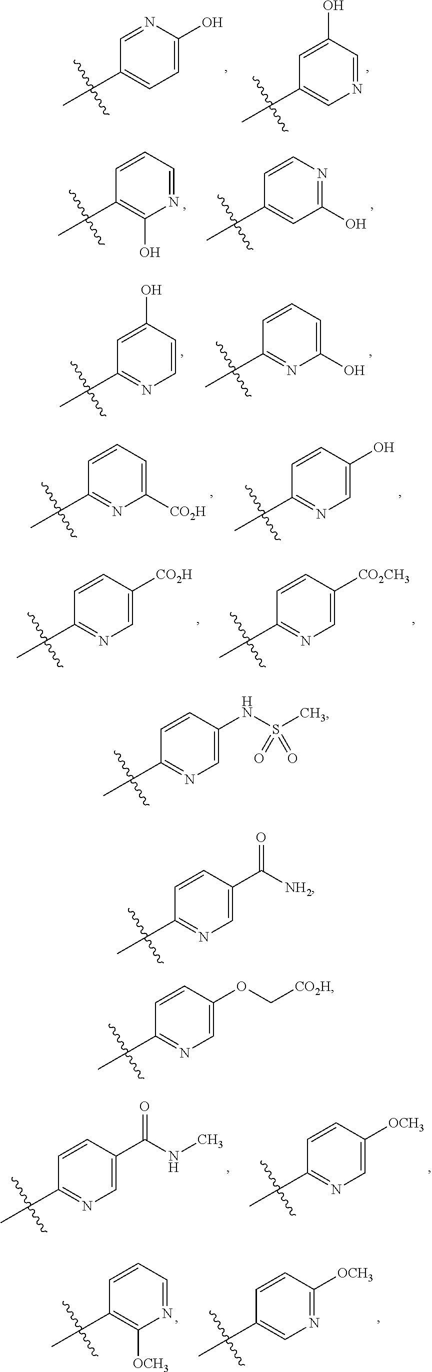 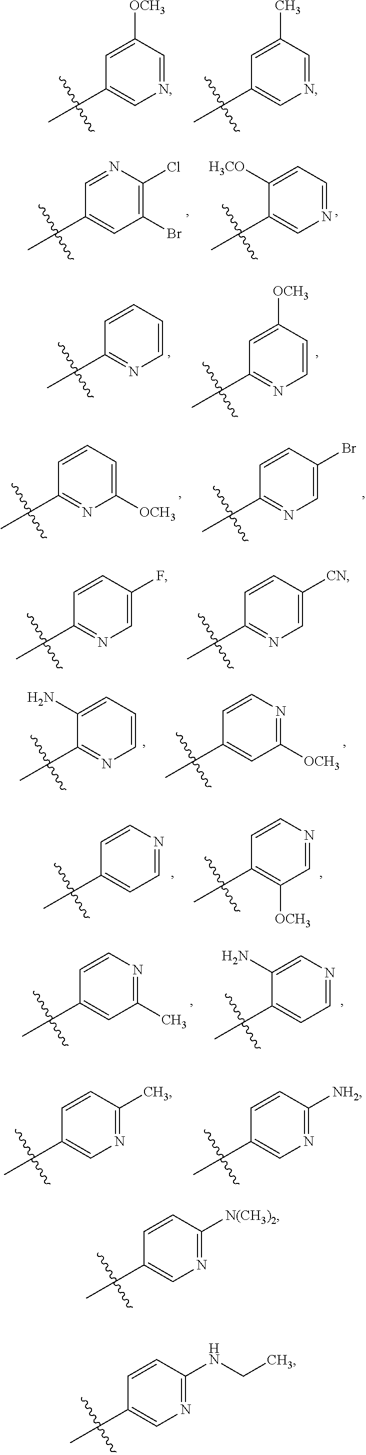 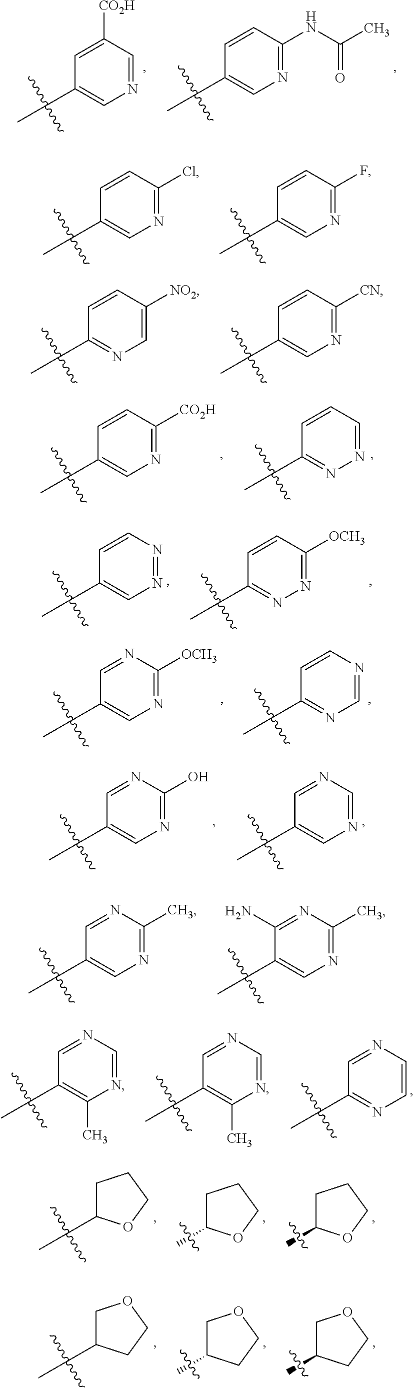 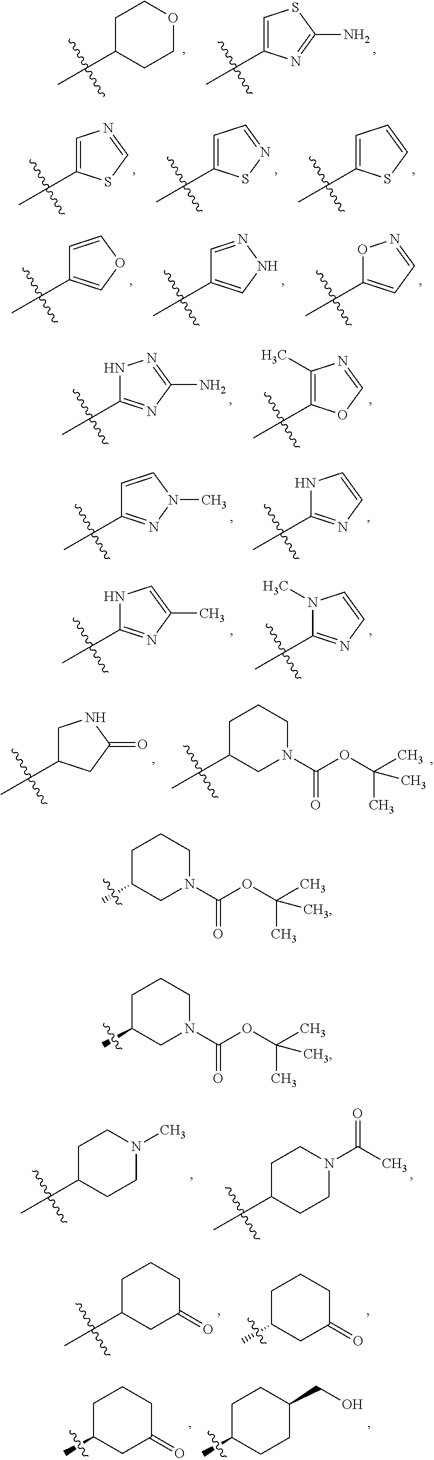 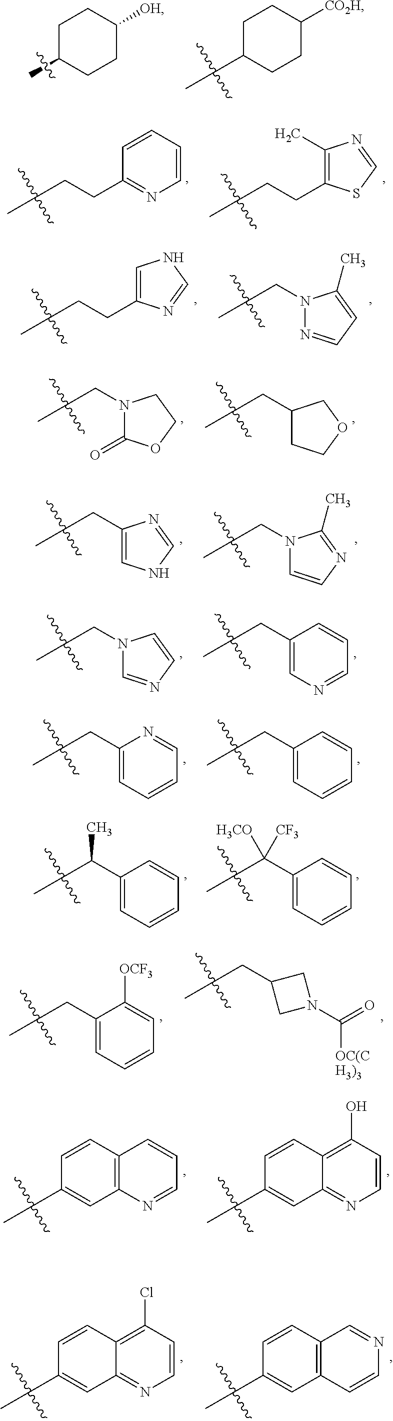 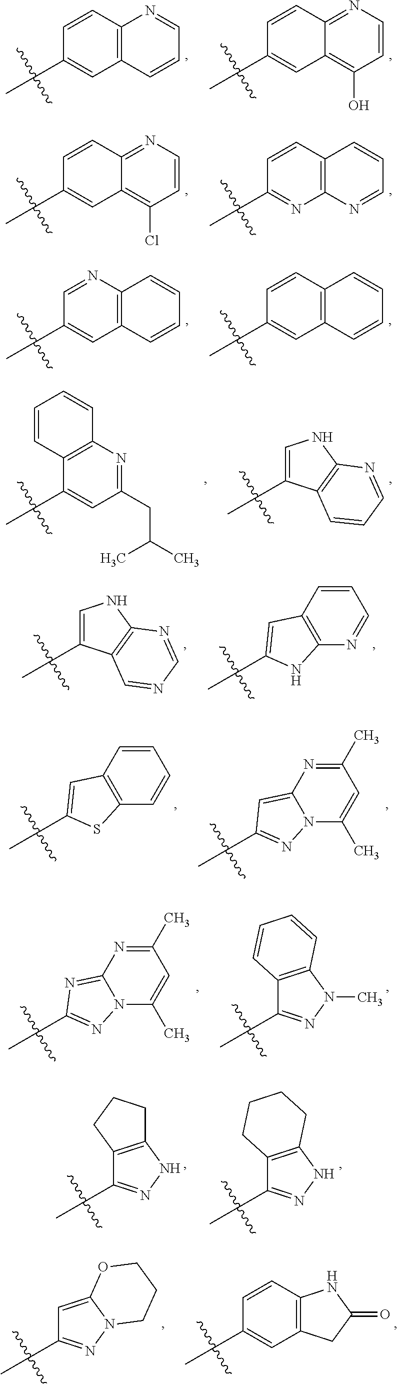 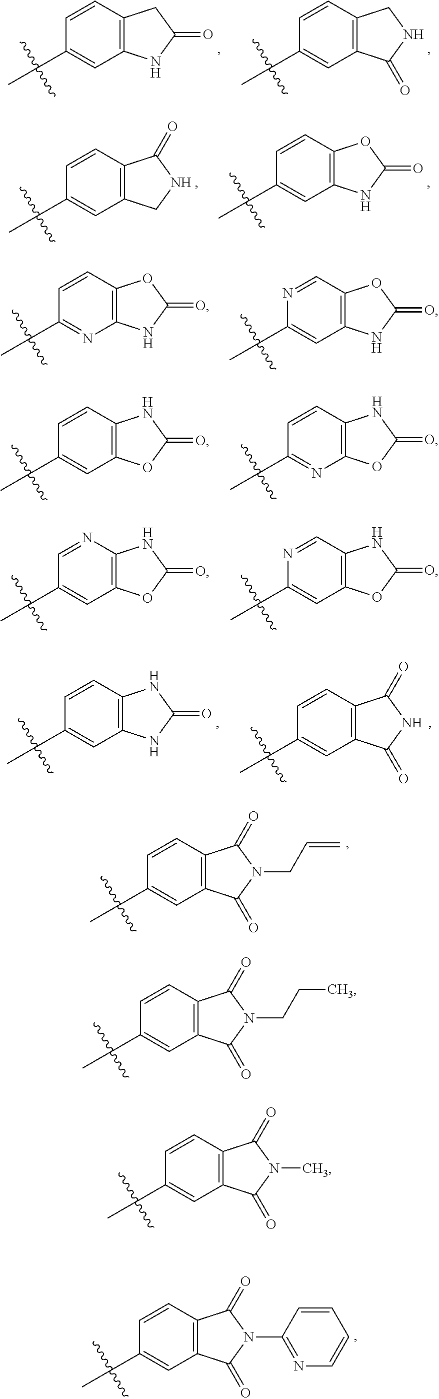 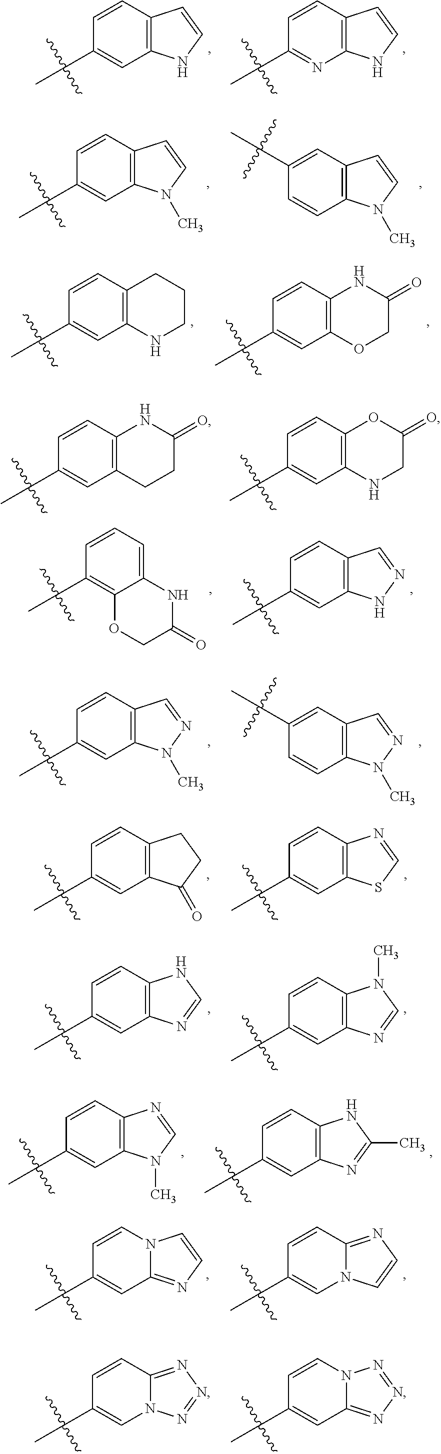 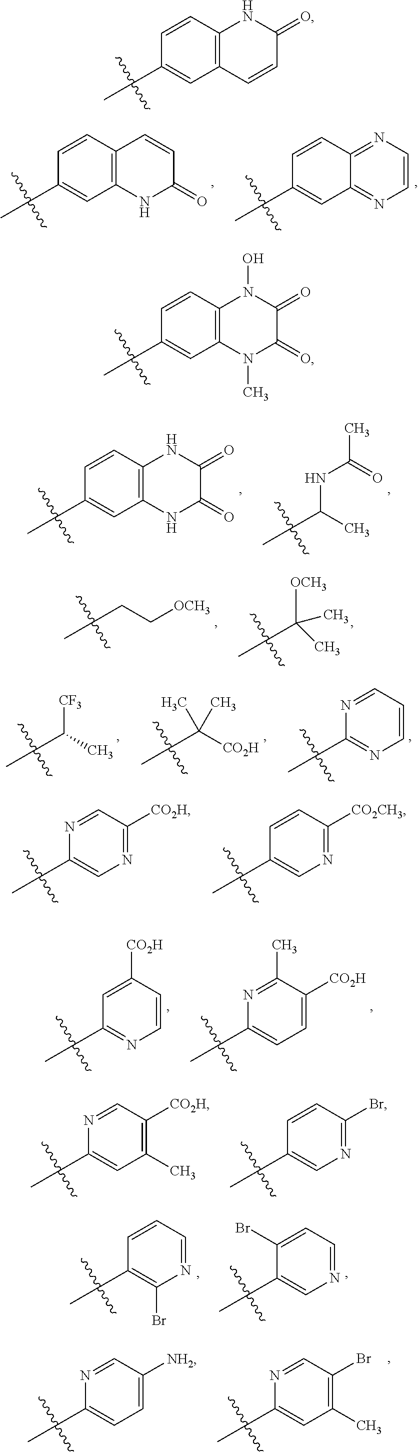 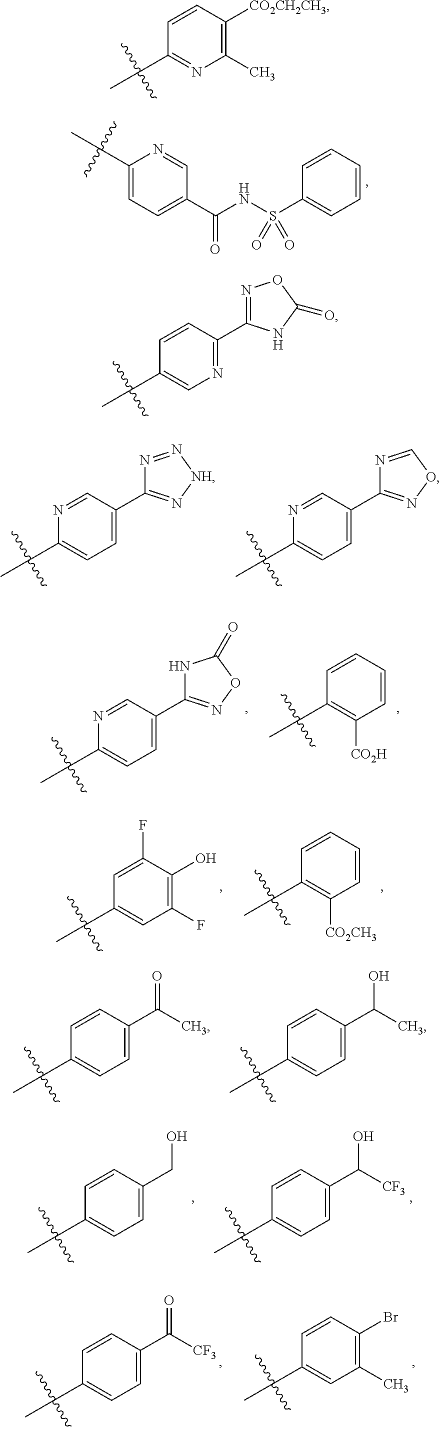 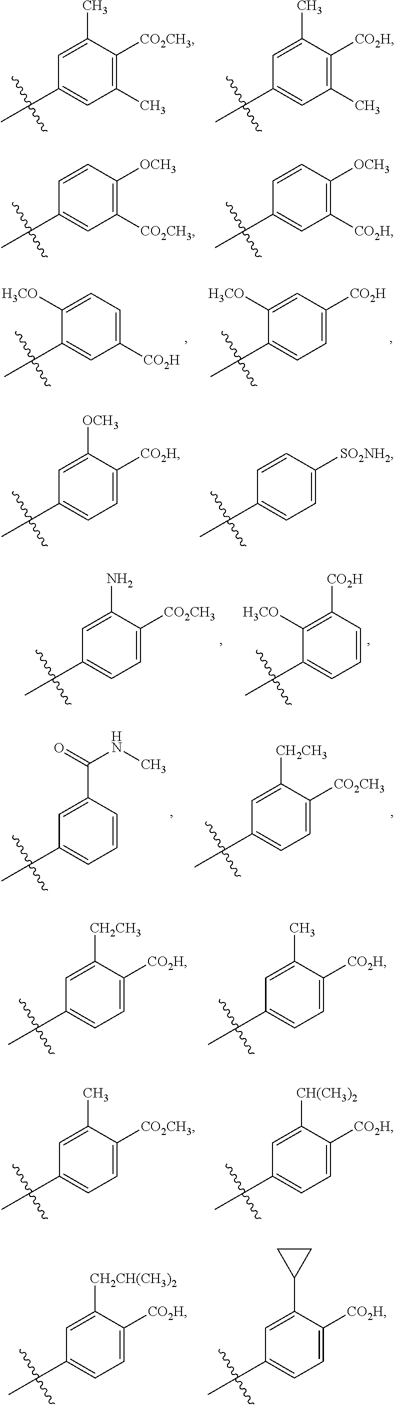 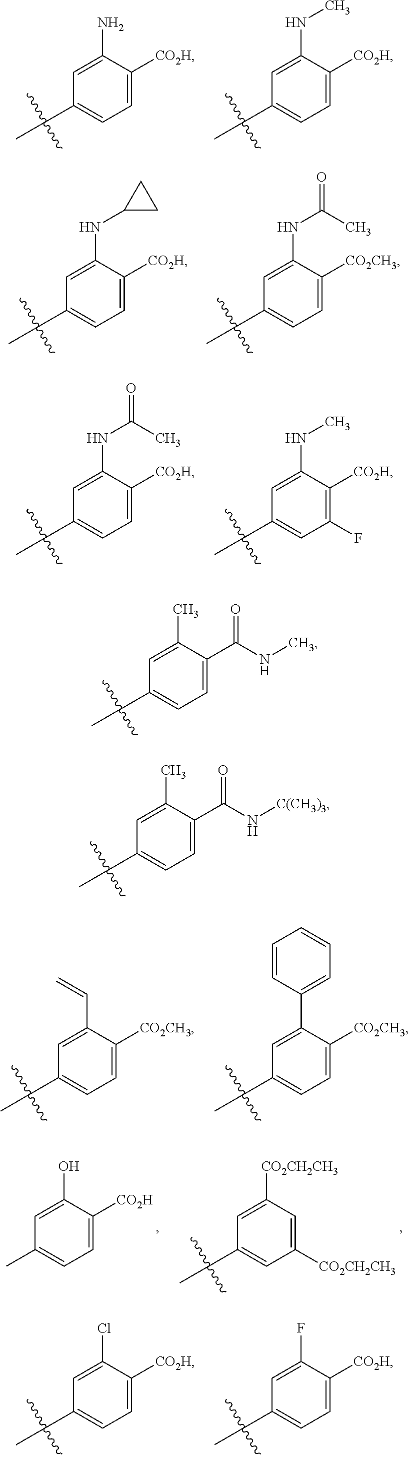 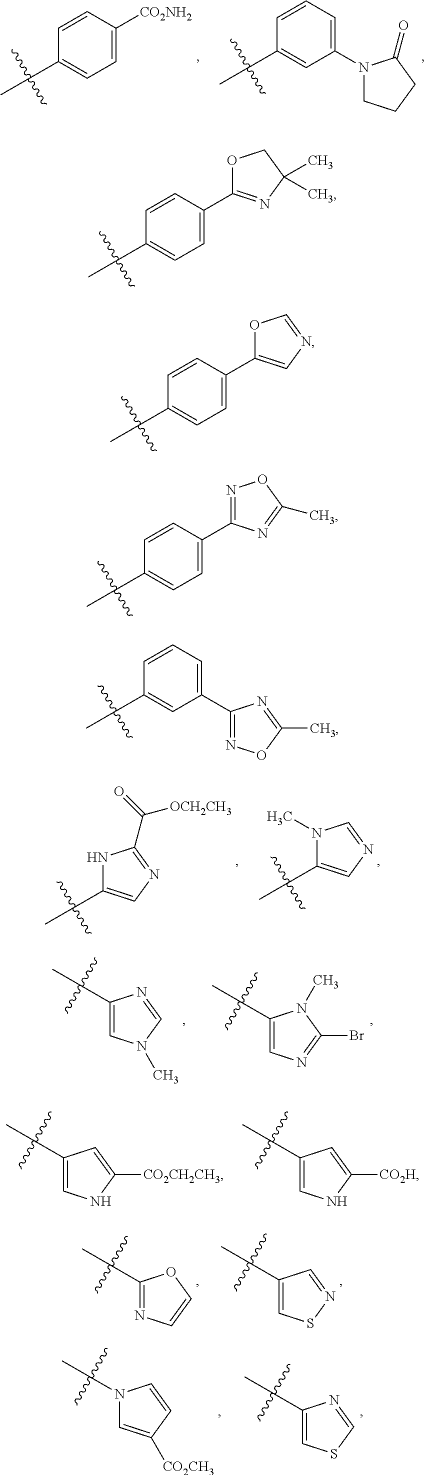 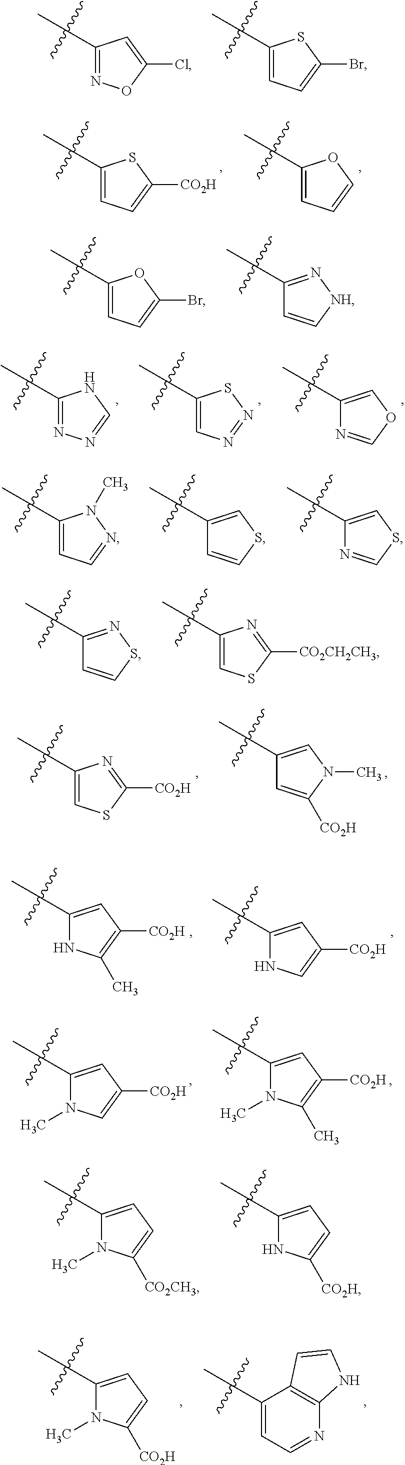 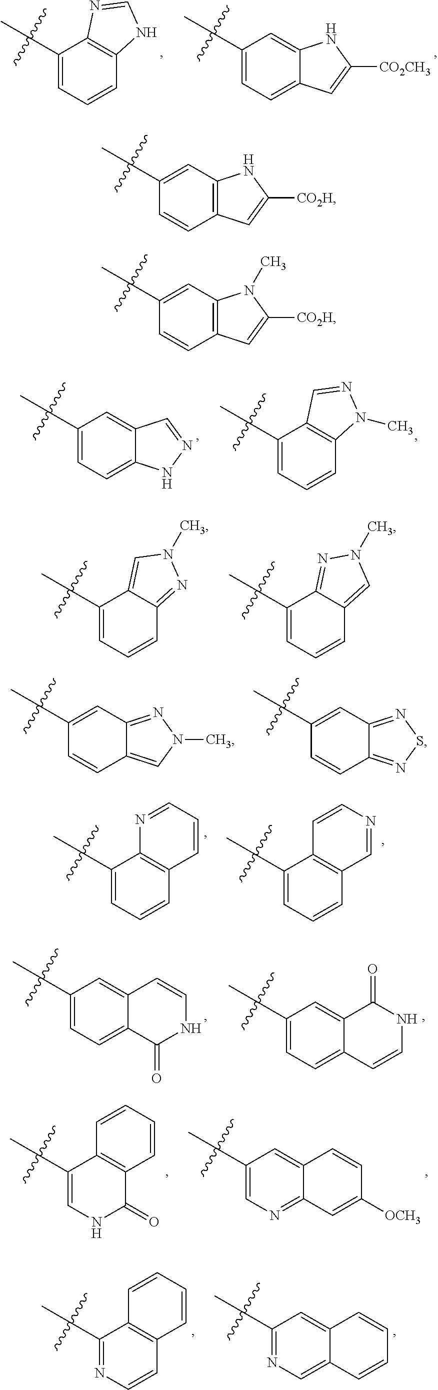 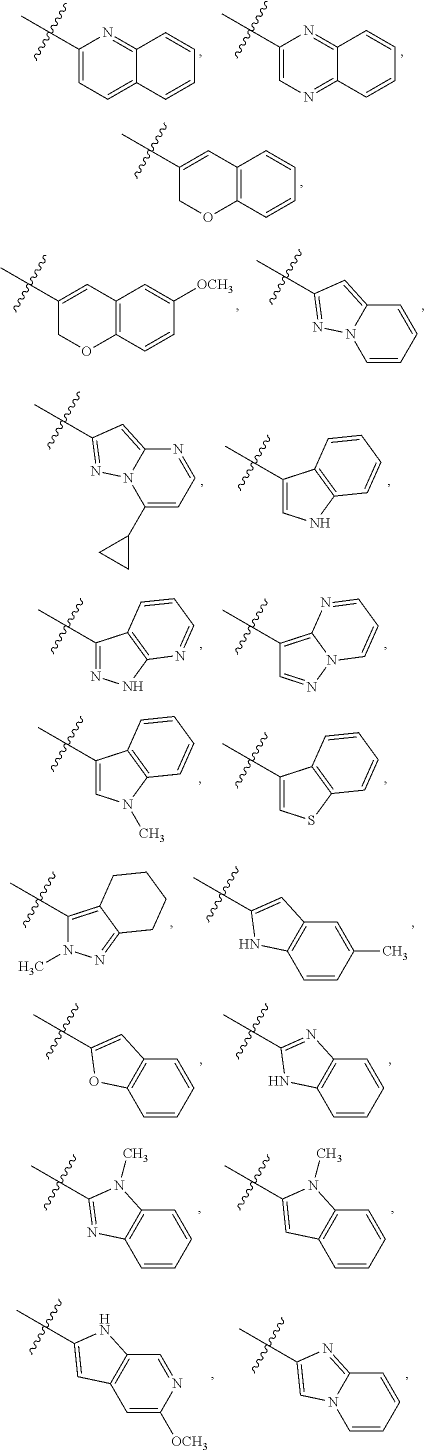 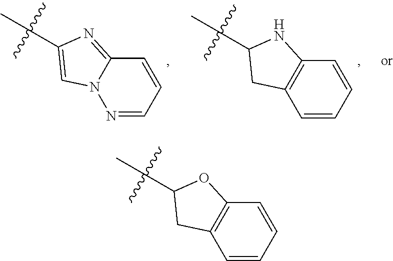 [SMILES: *c1cn2ncccc2n1 ; *C1Cc2ccccc2N1 ; *C1Cc2ccccc2O1]
the symbol [symbol] , when drawn across a bond, indicates the point of attachment to the rest of the molecule.

39. The compound of claim 1 or the pharmaceutically-acceptable salt thereof, the tautomer thereof, the pharmaceutically-acceptable salt of the tautomer, the stereoisomer thereof, or the mixture thereof, wherein R¹ is a group of formula 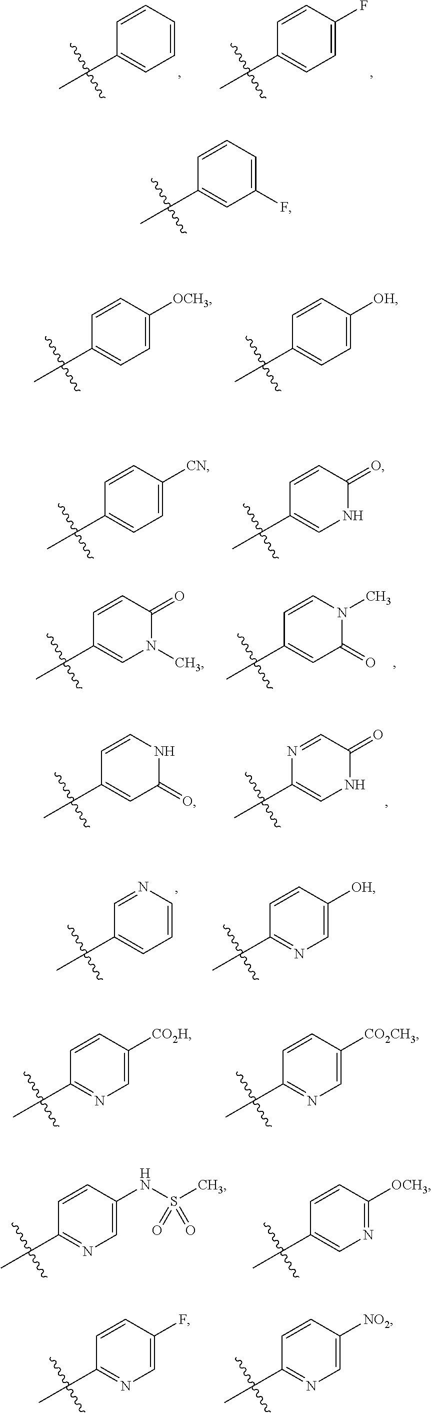 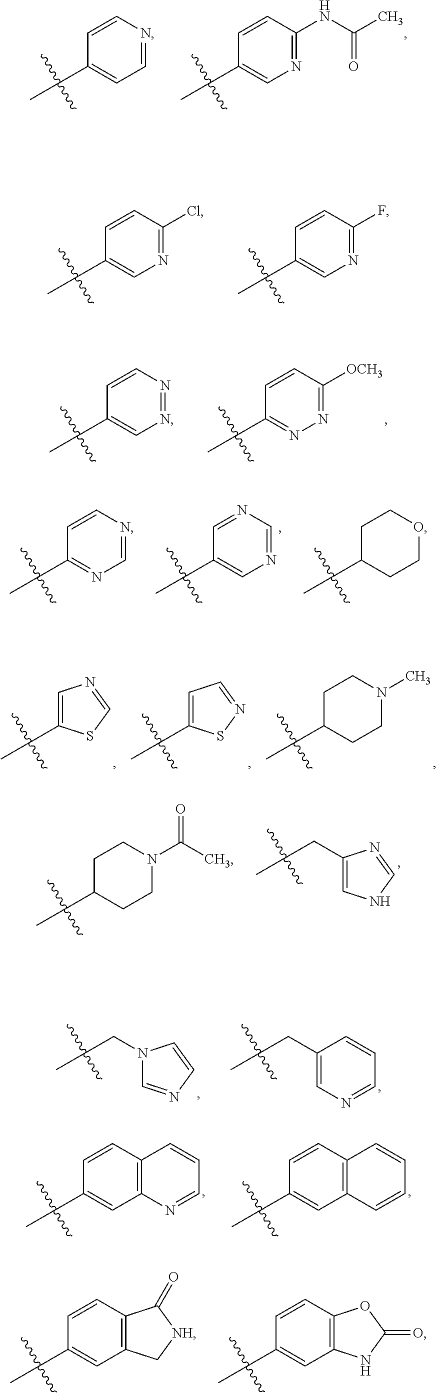 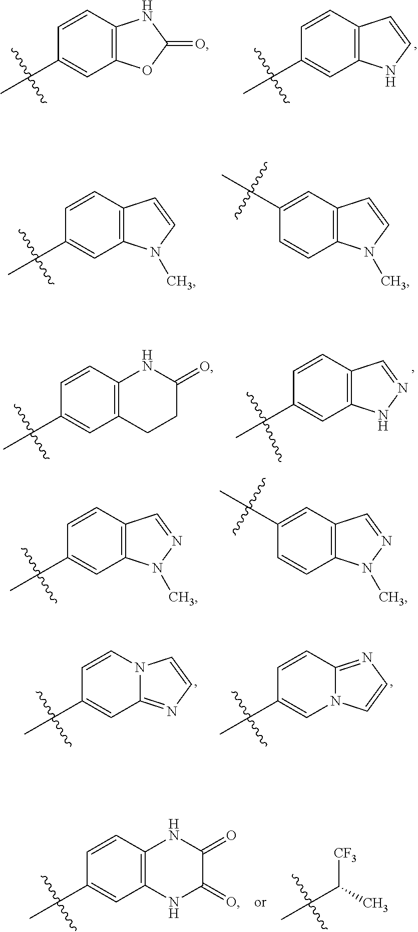
the symbol [symbol] , when drawn across a bond, indicates the point of attachment to the rest of the molecule.

40. The compound of claim 1 or the pharmaceutically-acceptable salt hereof, the tautomer thereof, the pharmaceutically-acceptable salt of the tautomer, the stereoisomer thereof, or the mixture thereof, wherein R¹ is a group of formula 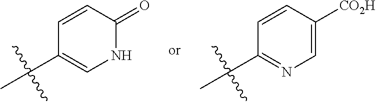 [SMILES: *c1ccc(=O)[nH]c1 ; *c1ccc(C(=O)O)cn1]
and the symbol [symbol] , when drawn across a bond, indicates the point of attachment to the rest of the molecule.

41. The compound of claim 1 or the pharmaceutically-acceptable salt thereof, the tautomer thereof, the pharmaceutically-acceptable salt of the tautomer, the stereoisomer thereof, or the mixture thereof, wherein R¹ is 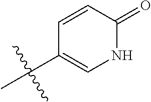 [SMILES: *c1ccc(=O)[nH]c1]
and the symbol [symbol] , when drawn across a bond, indicates the point of attachment to the rest of the molecule.

42. The compound of claim 1 or the pharmaceutically-acceptable salt thereof, the tautomer thereof, the pharmaceutically-acceptable salt of the tautomer, the stereoisomer thereof, or the mixture thereof, wherein R¹ is 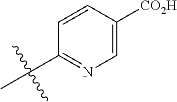 [SMILES: *c1ccc(C(=O)O)cn1]
and the symbol [symbol] , when drawn across a bond, indicates the point of attachment to the rest of the molecule.

43. The compound of claim 1 or the pharmaceutically-acceptable salt thereof, the tautomer thereof, the pharmaceutically-acceptable salt of the tautomer, the stereoisomer thereof, or the mixture thereof, wherein R¹ is 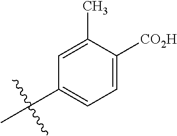 [SMILES: *c1ccc(C(=O)O)c(C)c1]
and the symbol [symbol] , when drawn across a bond, indicates the point of attachment to the rest of the molecule.

44. The compound of claim 1 or the pharmaceutically-acceptable salt thereof, the tautomer thereof, the pharmaceutically-acceptable salt of the tautomer, the stereoisomer thereof, or the mixture thereof, wherein R¹ is 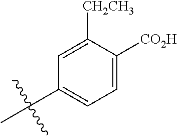 [SMILES: *c1ccc(C(=O)O)c(CC)c1]
and the symbol [symbol] , when drawn across a bond, indicates the point of attachment to the rest of the molecule.

45. The compound of claim 1 or the pharmaceutically-acceptable salt thereof, the tautomer thereof, the pharmaceutically-acceptable salt of the tautomer, the stereoisomer thereof, or the mixture thereof, wherein R¹ is 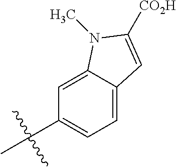 [SMILES: *c1ccc2cc(C(=O)O)n(C)c2c1]
and the symbol [symbol] , when drawn across a bond, indicates the point of attachment to the rest of the molecule.

46. The compound of claim 1, wherein the compound is
(S)-1-(4-(3-fluoro-4-(trifluoromethoxy)phenyl)-3,4-dihydro-2H-pyrano[3,2-b]pyridin-4-yl)-3-(pyrazin-2-yl)urea;
(S)-1-(4-(3-fluoro-4-(trifluoromethoxy)phenyl)-3,4-dihydro-2H-pyrano[3,2-b]pyridin-4-yl)-3-(pyrimidin-2-yl)urea;
(S)-methyl 6-(3-(4-(3-fluoro-4-(trifluoromethoxy)phenyl)-3,4-dihydro-2H-pyrano[3,2-b]pyridin-4-yl)ureido)nicotinate;
(S)-1-(4-(3-fluoro-4-(trifluoromethoxy)phenyl)-3,4-dihydro-2H-pyrano[3,2-b]pyridin-4-yl)-3-(pyrimidin-5-yl)urea;
(S)-methyl 2-(3-(4-(3-fluoro-4-(trifluoromethoxy)phenyl)-3,4-dihydro-2H-pyrano[3,2-b]pyridin-4-yl)ureido)benzoate;
(S)-methyl 5-(3-(4-(3-fluoro-4-(trifluoromethoxy)phenyl)-3,4-dihydro-2H-pyrano[3,2-b]pyridin-4-yl)ureido)picolinate;
(S)-1-(6-bromopyridin-3-yl)-3-(4-(3-fluoro-4-(trifluoromethoxy)phenyl)-3,4-dihydro-2H-pyrano[3,2-b]pyridin-4-yl)urea;
(S)-1-(4-(3-fluoro-4-(trifluoromethoxy)phenyl)-3,4-dihydro-2H-pyrano[3,2-b]pyridin-4-yl)-3-(pyrimidin-4-yl)urea;
(S)-methyl 4-(3-(4-(3-fluoro-4-(trifluoromethoxy)phenyl)-3,4-dihydro-2H-pyrano[3,2-b]pyridin-4-yl)ureido)benzoate;
(S)-methyl 3-(3-(4-(3-fluoro-4-(trifluoromethoxy)phenyl)-3,4-dihydro-2H-pyrano[3,2-b]pyridin-4-yl)ureido)benzoate;
(S)-1-(4-(3-fluoro-4-(trifluoromethoxy)phenyl)-3,4-dihydro-2H-pyrano[3,2-b]pyridin-4-yl)-3-(pyridin-3-yl)urea;
(S)-1-(2-bromopyridin-3-yl)-3-(4-(3-fluoro-4-(trifluoromethoxy)phenyl)-3,4-dihydro-2H-pyrano[3,2-b]pyridin-4-yl)urea;
(S)-1-(4-bromopyridin-3-yl)-3-(4-(3-fluoro-4-(trifluoromethoxy)phenyl)-3,4-dihydro-2H-pyrano[3,2-b]pyridin-4-yl)urea;
(S)-1-(4-(3-fluoro-4-(trifluoromethoxy)phenyl)-3,4-dihydro-2H-pyrano[3,2-b]pyridin-4-yl)-3-(pyridin-2-yl)urea;
(S)-1-(4-(3-fluoro-4-(trifluoromethoxy)phenyl)-3,4-dihydro-2H-pyrano[3,2-b]pyridin-4-yl)-3-(pyridin-4-yl)urea;
(S)-1-(3,5-difluoro-4-hydroxyphenyl)-3-(4-(3-fluoro-4-(trifluoromethoxy)phenyl)-3,4-dihydro-2H-pyrano[3,2-b]pyridin-4-yl)urea;
(S)—N-(4-(3-fluoro-4-(trifluoromethoxy)phenyl)-3,4-dihydro-2H-pyrano[3,2-b]pyridin-4-yl)piperidine-1-carboxamide;
(S)-methyl 1-((4-(3-fluoro-4-(trifluoromethoxy)phenyl)-3,4-dihydro-2H-pyrano[3,2-b]pyridin-4-yl)carbamoyl)piperidine-4-carboxylate;
(S)-4,4-difluoro-N-(4-(3-fluoro-4-(trifluoromethoxy)phenyl)-3,4-dihydro-2H-pyrano[3,2-b]pyridin-4-yl)piperidine-1-carboxamide;
(S)—N-(4-(3-fluoro-4-(trifluoromethoxy)phenyl)-3,4-dihydro-2H-pyrano[3,2-b]pyridin-4-yl)morpholine-4-carboxamide;
(S)—N-(4-(3-fluoro-4-(trifluoromethoxy)phenyl)-3,4-dihydro-2H-pyrano[3,2-b]pyridin-4-yl)-4-hydroxypiperidine-1-carboxamide;
(S)—N-(4-(3-fluoro-4-(trifluoromethoxy)phenyl)-3,4-dihydro-2H-pyrano[3,2-b]pyridin-4-yl)-4-oxopiperidine-1-carboxamide;
(S)—N-(4-(3-fluoro-4-(trifluoromethoxy)phenyl)-3,4-dihydro-2H-pyrano[3,2-b]pyridin-4-yl)pyrrolidine-1-carboxamide;
(S)—N-(4-(3-fluoro-4-(trifluoromethoxy)phenyl)-3,4-dihydro-2H-pyrano[3,2-b]pyridin-4-yl)azetidine-1-carboxamide;
(S)-2-(3-(4-(3-fluoro-4-(trifluoromethoxy)phenyl)-3,4-dihydro-2H-pyrano[3,2-b]pyridin-4-yl)ureido)benzoic acid;
(S)-3-(3-(4-(3-fluoro-4-(trifluoromethoxy)phenyl)-3,4-dihydro-2H-pyrano[3,2-b]pyridin-4-yl)ureido)benzoic acid;
(S)-4-(3-(4-(3-fluoro-4-(trifluoromethoxy)phenyl)-3,4-dihydro-2H-pyrano[3,2-b]pyridin-4-yl)ureido)benzoic acid;
(S)-1-(4-(3-fluoro-4-(trifluoromethoxy)phenyl)-3,4-dihydro-2H-pyrano[3,2-b]pyridin-4-yl)-3-(6-(5-oxo-4,5-dihydro-1,2,4-oxadiazol-3-yl)pyridin-3-yl)urea;
(S)-1-((4-(3-fluoro-4-(trifluoromethoxy)phenyl)-3,4-dihydro-2H-pyrano[3,2-b]pyridin-4-yl)carbamoyl)piperidine-4-carboxylic acid;
(S)—N-(4-(4-(cyanomethyl)phenyl)-3,4-dihydro-2H-pyrano[3,2-b]pyridin-4-yl)-4-fluorobenzamide;
(S)-4-fluoro-N-(4-(4-(pyridin-2-yl)phenyl)-3,4-dihydro-2H-pyrano[3,2-b]pyridin-4-yl)benzamide;
(S)—N-(4-([1,1′-biphenyl]-4-yl)-3,4-dihydro-2H-pyrano[3,2-b]pyridin-4-yl)-4-fluorobenzamide;
(S)-4-fluoro-N-(4-(4′-fluoro-[1,1′-biphenyl]-4-yl)-3,4-dihydro-2H-pyrano[3,2-b]pyridin-4-yl)benzamide;
(S)-4-fluoro-N-(4-(4-(pyridin-3-yl)phenyl)-3,4-dihydro-2H-pyrano[3,2-b]pyridin-4-yl)benzamide;
(S)-4-fluoro-N-(4-(4-(pyridin-4-yl)phenyl)-3,4-dihydro-2H-pyrano[3,2-b]pyridin-4-yl)benzamide;
(S)—N-(4-(4-(5-ethoxypyrazin-2-yl)phenyl)-3,4-dihydro-2H-pyrano[3,2-b]pyridin-4-yl)-4-fluorobenzamide;
(S)-4-fluoro-N-(4-(3′-(hydroxymethyl)-[1,1′-biphenyl]-4-yl)-3,4-dihydro-2H-pyrano[3,2-b]pyridin-4-yl)benzamide;
(S)-4-fluoro-N-(4-(4′-(methylsulfonyl)-[1,1′-biphenyl]-4-yl)-3,4-dihydro-2H-pyrano[3,2-b]pyridin-4-yl)benzamide;
(S)-4-fluoro-N-(4-(2′-hydroxy-[1,1′-biphenyl]-4-yl)-3,4-dihydro-2H-pyrano[3,2-b]pyridin-4-yl)benzamide;
(S)—N-(4-(4′-acetamido-[1,1′-biphenyl]-4-yl)-3,4-dihydro-2H-pyrano[3,2-b]pyridin-4-yl)-4-fluorobenzamide;
(S)-4-fluoro-N-(4-(4′-fluoro-3′-(hydroxymethyl)-[1,1′-biphenyl]-4-yl)-3,4-dihydro-2H-pyrano[3,2-b]pyridin-4-yl)benzamide;
(S)-4-chloro-4′-(4-(4-fluorobenzamido)-3,4-dihydro-2H-pyrano[3,2-b]pyridin-4-yl)-[1,1′-biphenyl]-3-carboxamide;
(S)-4-fluoro-N-(4-(4-(pyrimidin-5-yl)phenyl)-3,4-dihydro-2H-pyrano[3,2-b]pyridin-4-yl)benzamide;
(S)—N-(4-(4-(3,5-dimethylisoxazol-4-yl)phenyl)-3,4-dihydro-2H-pyrano[3,2-b]pyridin-4-yl)-4-fluorobenzamide;
(S)—N-(4-(3′-amino-[1,1′-biphenyl]-4-yl)-3,4-dihydro-2H-pyrano[3,2-b]pyridin-4-yl)-4-fluorobenzamide;
(S)-4-fluoro-N-(4-(4-(2-methoxypyrimidin-5-yl)phenyl)-3,4-dihydro-2H-pyrano[3,2-b]pyridin-4-yl)benzamide;
(S)-4-fluoro-N-(4-(2′-(hydroxymethyl)-[1,1′-biphenyl]-4-yl)-3,4-dihydro-2H-pyrano[3,2-b]pyridin-4-yl)benzamide;
(S)-4′-(4-(4-fluorobenzamido)-3,4-dihydro-2H-pyrano[3,2-b]pyridin-4-yl)-N,N-dimethyl-[1,1′-biphenyl]-3-carboxamide;
(S)-4-fluoro-N-(4-(4-(4-methoxypyridin-3-yl)phenyl)-3,4-dihydro-2H-pyrano[3,2-b]pyridin-4-yl)benzamide;
(S)-4-fluoro-N-(4-(4-(2-methoxypyridin-3-yl)phenyl)-3,4-dihydro-2H-pyrano[3,2-b]pyridin-4-yl)benzamide;
(S)-4-fluoro-N-(4-(4-(4-methylpyridin-3-yl)phenyl)-3,4-dihydro-2H-pyrano[3,2-b]pyridin-4-yl)benzamide;
(S)-4-fluoro-N-(4-(4′-morpholino-[1,1′-biphenyl]-4-yl)-3,4-dihydro-2H-pyrano[3,2-b]pyridin-4-yl)benzamide;
(S)-4-fluoro-N-(4-(3′-fluoro-2′-hydroxy-[1,1′-biphenyl]-4-yl)-3,4-dihydro-2H-pyrano[3,2-b]pyridin-4-yl)benzamide;
(S)-4-fluoro-N-(4-(2′-hydroxy-3′-methoxy-[1,1′-biphenyl]-4-yl)-3,4-dihydro-2H-pyrano[3,2-b]pyridin-4-yl)benzamide;
(S)-4-fluoro-N-(4-(2′-(hydroxymethyl)-4′-methoxy-[1,1′-biphenyl]-4-yl)-3,4-dihydro-2H-pyrano[3,2-b]pyridin-4-yl)benzamide;
(S)-4′-(4-(4-fluorobenzamido)-3,4-dihydro-2H-pyrano[3,2-b]pyridin-4-yl)-N,N-dimethyl-[1,1′-biphenyl]-4-carboxamide;
(S)-4′-(4-(4-fluorobenzamido)-3,4-dihydro-2H-pyrano[3,2-b]pyridin-4-yl)-N-methyl-[1,1′-biphenyl]-4-carboxamide;
(S)—N-(4-(4-(2-chloropyrimidin-5-yl)phenyl)-3,4-dihydro-2H-pyrano[3,2-b]pyridin-4-yl)-4-fluorobenzamide;
(S)-4-fluoro-N-(4-(5′-fluoro-2′-(hydroxymethyl)-[1,1′-biphenyl]-4-yl)-3,4-dihydro-2H-pyrano[3,2-b]pyridin-4-yl)benzamide;
(S)—N-(4-(4-(5-cyanopyridin-3-yl)phenyl)-3,4-dihydro-2H-pyrano[3,2-b]pyridin-4-yl)-4-fluorobenzamide;
(S)-4-fluoro-N-(4-(3′-morpholino-[1,1′-biphenyl]-4-yl)-3,4-dihydro-2H-pyrano[3,2-b]pyridin-4-yl)benzamide;
(S)—N-(4-(2′-amino-[1,1′-biphenyl]-4-yl)-3,4-dihydro-2H-pyrano[3,2-b]pyridin-4-yl)-4-fluorobenzamide;
(S)-4-fluoro-N-(4-(2′-(methylsulfonyl)-[1,1′-biphenyl]-4-yl)-3,4-dihydro-2H-pyrano[3,2-b]pyridin-4-yl)benzamide;
(S)-4-fluoro-N-(4-(4-(furan-3-yl)phenyl)-3,4-dihydro-2H-pyrano[3,2-b]pyridin-4-yl)benzamide;
(S)-4-fluoro-N-(4-(4-(2-methoxypyridin-4-yl)phenyl)-3,4-dihydro-2H-pyrano[3,2-b]pyridin-4-yl)benzamide;
(S)—N-(4-(4′-amino-[1,1′-biphenyl]-4-yl)-3,4-dihydro-2H-pyrano[3,2-b]pyridin-4-yl)-4-fluorobenzamide;
(S)—N-(4-(3′-amino-4′-methyl-[1,1′-biphenyl]-4-yl)-3,4-dihydro-2H-pyrano[3,2-b]pyridin-4-yl)-4-fluorobenzamide;
(S)—N-(4-(4-(2,4-dimethoxypyrimidin-5-yl)phenyl)-3,4-dihydro-2H-pyrano[3,2-b]pyridin-4-yl)-4-fluorobenzamide;
(S)-4-fluoro-N-(4-(3′-(5-methyl-1,3,4-oxadiazol-2-yl)-[1,1′-biphenyl]-4-yl)-3,4-dihydro-2H-pyrano[3,2-b]pyridin-4-yl)benzamide;
(S)-4-fluoro-N-(4-(4′-(hydroxymethyl)-[1,1′-biphenyl]-4-yl)-3,4-dihydro-2H-pyrano[3,2-b]pyridin-4-yl)benzamide;
(R)—N—((S)-4-(3-fluoro-4-(trifluoromethoxy)phenyl)-3,4-dihydro-2H-pyrano[3,2-b]pyridin-4-yl)-2-methylpropane-2-sulfinamide;
(S)—N-(4-(4′-cyano-[1,1′-biphenyl]-4-yl)-3,4-dihydro-2H-pyrano[3,2-b]pyridin-4-yl)-4-fluorobenzamide;
(S)—N-(4-(4′-cyano-[1,1′-biphenyl]-4-yl)-3,4-dihydro-2H-pyrano[3,2-b]pyridin-4-yl)benzamide;
(S)—N-(4-(4′-cyano-[1,1′-biphenyl]-4-yl)-3,4-dihydro-2H-pyrano[3,2-b]pyridin-4-yl)-2-oxo-2,3-dihydrobenzo[d]oxazole-5-carboxamide 2,2,2-trifluoroacetate;
(S)-4-acetyl-N-(4-(3-fluoro-4-(trifluoromethoxy)phenyl)-3,4-dihydro-2H-pyrano[3,2-b]pyridin-4-yl)benzamide;
N—((S)-4-(3-fluoro-4-(trifluoromethoxy)phenyl)-3,4-dihydro-2H-pyrano[3,2-b]pyridin-4-yl)-4-((R)-1-hydroxyethyl)benzamide and N—((S)-4-(3-fluoro-4-(trifluoromethoxy)phenyl)-3,4-dihydro-2H-pyrano[3,2-b]pyridin-4-yl)-4-((S)-1-hydroxyethyl)benzamide;
(S)—N-(4-(3-fluoro-4-(trifluoromethoxy)phenyl)-3,4-dihydro-2H-pyrano[3,2-b]pyridin-4-yl)-4-(hydroxymethyl)benzamide;
N—((S)-4-(3-fluoro-4-(trifluoromethoxy)phenyl)-3,4-dihydro-2H-pyrano[3,2-b]pyridin-4-yl)-4-((R)-2,2,2-trifluoro-1-hydroxyethyl)benzamide and N—((S)-4-(3-fluoro-4-(trifluoromethoxy)phenyl)-3,4-dihydro-2H-pyrano[3,2-b]pyridin-4-yl)-4-((S)-2,2,2-trifluoro-1-hydroxyethyl)benzamide;
(S)—N-(4-(3-fluoro-4-(trifluoromethoxy)phenyl)-3,4-dihydro-2H-pyrano[3,2-b]pyridin-4-yl)-4-(2,2,2-trifluoroacetyl)benzamide;
(R)-6-((3-(3-Fluoro-4-(trifluoromethoxy)phenyl)-2,3-dihydrofuro[3,2-b]pyridin-3-yl)carbamoyl)nicotinic acid;
(S)-ethyl 5-((4-(3-fluoro-4-(trifluoromethoxy)phenyl)-3,4-dihydro-2H-pyrano[3,2-b]pyridin-4-yl)carbamoyl)-1H-imidazole-2-carboxylate;
(S)—N-(4-(3-fluoro-4-(trifluoromethoxy)phenyl)-3,4-dihydro-2H-pyrano[3,2-b]pyridin-4-yl)-1-methyl-1H-imidazole-5-carboxamide;
(S)—N-(8-(3-fluoro-4-(trifluoromethoxy)phenyl)-7,8-dihydro-6H-pyrano[2,3-b]pyrazin-8-yl)-6-oxo-1,6-dihydropyridine-3-carboxamide;
(S)—N-(7-(3-fluoro-4-(trifluoromethoxy)phenyl)-5-oxo-6,7-dihydro-5H-cyclopenta[b]pyridin-7-yl)-6-oxo-1,6-dihydropyridine-3-carboxamide;
(S)-5-bromo-N-(4-(3-fluoro-4-(trifluoromethoxy)phenyl)-3,4-dihydro-2H-pyrano[3,2-b]pyridin-4-yl)-4-methylpicolinamide;
(S)-4-bromo-N-(4-(3-fluoro-4-(trifluoromethoxy)phenyl)-3,4-dihydro-2H-pyrano[3,2-b]pyridin-4-yl)-3-methylbenzamide;
(S)-4-(4,4-dimethyl-4,5-dihydrooxazol-2-yl)-N-(4-(3-fluoro-4-(trifluoromethoxy)phenyl)-3,4-dihydro-2H-pyrano[3,2-b]pyridin-4-yl)benzamide;
(S)-ethyl 6-((4-(3-fluoro-4-(trifluoromethoxy)phenyl)-3,4-dihydro-2H-pyrano[3,2-b]pyridin-4-yl)carbamoyl)-2-methylnicotinate;
(S)-5-cyano-N-(4-(3-fluoro-4-(trifluoromethoxy)phenyl)-3,4-dihydro-2H-pyrano[3,2-b]pyridin-4-yl)picolinamide;
(S)-methyl 6-((4-(3-fluoro-4-(trifluoromethyl)phenyl)-3,4-dihydro-2H-pyrano[3,2-b]pyridin-4-yl)carbamoyl)nicotinate;
(S)-methyl 6-((4-(2-fluoro-4-(trifluoromethoxy)phenyl)-3,4-dihydro-2H-pyrano[3,2-b]pyridin-4-yl)carbamoyl)nicotinate;
(S)-methyl 6-((4-(4-(trifluoromethyl)phenyl)-3,4-dihydro-2H-pyrano[3,2-b]pyridin-4-yl)carbamoyl)nicotinate;
(S)-methyl 6-((4-(4-(trifluoromethoxy)phenyl)-3,4-dihydro-2H-pyrano[3,2-b]pyridin-4-yl)carbamoyl)nicotinate;
(S)-5-((4-(3-fluoro-4-(trifluoromethoxy)phenyl)-3,4-dihydro-2H-pyrano[3,2-b]pyridin-4-yl)carbamoyl)pyrazine-2-carboxylic acid;
(S)-3-((4-(3-fluoro-4-(trifluoromethoxy)phenyl)-3,4-dihydro-2H-pyrano[3,2-b]pyridin-4-yl)carbamoyl)benzoic acid;
(S)—N-(4-(3-fluoro-4-(trifluoromethoxy)phenyl)-3,4-dihydro-2H-pyrano[3,2-b]pyridin-4-yl)-1-methyl-1H-imidazole-4-carboxamide;
(S)-2-bromo-N-(4-(3-fluoro-4-(trifluoromethoxy)phenyl)-3,4-dihydro-2H-pyrano[3,2-b]pyridin-4-yl)-1-methyl-1H-imidazole-5-carboxamide;
(S)-methyl 4-((4-(3-fluoro-4-(trifluoromethoxy)phenyl)-3,4-dihydro-2H-pyrano[3,2-b]pyridin-4-yl)carbamoyl)-2,6-dimethylbenzoate;
(S)-methyl 5-((4-(3-fluoro-4-(trifluoromethoxy)phenyl)-3,4-dihydro-2H-pyrano[3,2-b]pyridin-4-yl)carbamoyl)-2-methoxybenzoate;
(S)-methyl 6-((4-(3,4-dichlorophenyl)-3,4-dihydro-2H-pyrano[3,2-b]pyridin-4-yl)carbamoyl)nicotinate;
(S)—N-(4-(3-fluoro-4-(trifluoromethoxy)phenyl)-3,4-dihydro-2H-pyrano[3,2-b]pyridin-4-yl)-4-sulfamoylbenzamide;
(S)—N-(4-(4-fluoro-3-(trifluoromethyl)phenyl)-3,4-dihydro-2H-pyrano[3,2-b]pyridin-4-yl)-6-oxo-1,6-dihydropyridine-3-carboxamide;
(S)-3-((4-(3-fluoro-4-(trifluoromethoxy)phenyl)-3,4-dihydro-2H-pyrano[3,2-b]pyridin-4-yl)carbamoyl)-2-methoxybenzoic acid;
(S)-ethyl 4-((4-(3-fluoro-4-(trifluoromethoxy)phenyl)-3,4-dihydro-2H-pyrano[3,2-b]pyridin-4-yl)carbamoyl)-1H-pyrrole-2-carboxylate;
(S)-methyl 2-amino-4-((4-(3-fluoro-4-(trifluoromethoxy)phenyl)-3,4-dihydro-2H-pyrano[3,2-b]pyridin-4-yl)carbamoyl)benzoate;
(S)—N-(4-(3-fluoro-4-(trifluoromethoxy)phenyl)-3,4-dihydro-2H-pyrano[3,2-b]pyridin-4-yl)oxazole-2-carboxamide;
(S)—N-(4-(3-fluoro-4-(trifluoromethoxy)phenyl)-3,4-dihydro-2H-pyrano[3,2-b]pyridin-4-yl)isothiazole-4-carboxamide;
(S)-methyl 2-ethyl-4-((4-(3-fluoro-4-(trifluoromethoxy)phenyl)-3,4-dihydro-2H-pyrano[3,2-b]pyridin-4-yl)carbamoyl)benzoate;
(S)-diethyl 5-((4-(3-fluoro-4-(trifluoromethoxy)phenyl)-3,4-dihydro-2H-pyrano[3,2-b]pyridin-4-yl)carbamoyl)isophthalate;
(S)-methyl 1-(2-((4-(3-fluoro-4-(trifluoromethoxy)phenyl)-3,4-dihydro-2H-pyrano[3,2-b]pyridin-4-yl)amino)-2-oxoethyl)-1H-pyrrole-3-carboxylate;
(S)—N1-(4-(3-fluoro-4-(trifluoromethoxy)phenyl)-3,4-dihydro-2H-pyrano[3,2-b]pyridin-4-yl)terephthalamide;
(S)-methyl 6-((4-(3-fluoro-4-(trifluoromethoxy)phenyl)-3,4-dihydro-2H-pyrano[3,2-b]pyridin-4-yl)carbamoyl)-1-methyl-1H-indole-2-carboxylate;
(S)-2-chloro-4-((4-(3-fluoro-4-(trifluoromethoxy)phenyl)-3,4-dihydro-2H-pyrano[3,2-b]pyridin-4-yl)carbamoyl)benzoic acid;
(S)—N-(4-(3-fluoro-4-(trifluoromethoxy)phenyl)-3,4-dihydro-2H-pyrano[3,2-b]pyridin-4-yl)thiazole-4-carboxamide;
(S)—N-(4-(3-methyl-4-(trifluoromethoxy)phenyl)-3,4-dihydro-2H-pyrano[3,2-b]pyridin-4-yl)-6-oxo-1,6-dihydropyridine-3-carboxamide;
(S)-5-chloro-N-(4-(3-fluoro-4-(trifluoromethoxy)phenyl)-3,4-dihydro-2H-pyrano[3,2-b]pyridin-4-yl)isoxazole-3-carboxamide;
(S)-methyl 2-((4-(3-fluoro-4-(trifluoromethoxy)phenyl)-3,4-dihydro-2H-pyrano[3,2-b]pyridin-4-yl)carbamoyl)isonicotinate;
N—((S)-4-(3-fluoro-4-(trifluoromethoxy)phenyl)-3,4-dihydro-2H-pyrano[3,2-b]pyridin-4-yl)-1-((R)-2-hydroxypropyl)-6-oxo-1,6-dihydropyridine-3-carboxamide;
(S)-5-bromo-N-(4-(3-fluoro-4-(trifluoromethoxy)phenyl)-3,4-dihydro-2H-pyrano[3,2-b]pyridin-4-yl)thiophene-2-carboxamide;
(S)-4-((4-(3-fluoro-4-(trifluoromethoxy)phenyl)-3,4-dihydro-2H-pyrano[3,2-b]pyridin-4-yl)carbamoyl)benzoic acid;
(S)-3,5-difluoro-4-hydroxy-N-(4-(4-(trifluoromethyl)phenyl)-3,4-dihydro-2H-pyrano[3,2-b]pyridin-4-yl)benzamide;
(S)-5-bromo-N-(4-(3-fluoro-4-(trifluoromethoxy)phenyl)-3,4-dihydro-2H-pyrano[3,2-b]pyridin-4-yl)picolinamide;
(S)-3,5-difluoro-N-(4-(3-fluoro-4-(trifluoromethoxy)phenyl)-3,4-dihydro-2H-pyrano[3,2-b]pyridin-4-yl)-4-hydroxybenzamide;
(S)-5-bromo-N-(4-(3-fluoro-4-(trifluoromethoxy)phenyl)-3,4-dihydro-2H-pyrano[3,2-b]pyridin-4-yl)furan-2-carboxamide;
(S)-methyl 6-((4-(3-fluoro-4-(trifluoromethoxy)phenyl)-3,4-dihydro-2H-pyrano[3,2-b]pyridin-4-yl)carbamoyl)-1H-indole-2-carboxylate;
(S)—N-(4-(4-(trifluoromethoxy)phenyl)-3,4-dihydro-2H-pyrano[3,2-b]pyridin-4-yl)-1H-imidazole-2-carboxamide;
(S)—N-(4-(4-(trifluoromethoxy)phenyl)-3,4-dihydro-2H-pyrano[3,2-b]pyridin-4-yl)-1H-pyrazole-3-carboxamide;
(S)—N-(4-(4-(trifluoromethoxy)phenyl)-3,4-dihydro-2H-pyrano[3,2-b]pyridin-4-yl)-4H-1,2,4-triazole-3-carboxamide;
(S)—N-(4-(4-(trifluoromethoxy)phenyl)-3,4-dihydro-2H-pyrano[3,2-b]pyridin-4-yl)isothiazole-5-carboxamide;
(S)—N-(4-(4-(trifluoromethoxy)phenyl)-3,4-dihydro-2H-pyrano[3,2-b]pyridin-4-yl)-1,2,3-thiadiazole-5-carboxamide;
(S)—N-(4-(4-(trifluoromethoxy)phenyl)-3,4-dihydro-2H-pyrano[3,2-b]pyridin-4-yl)-1H-indazole-5-carboxamide;
(S)—N-(4-(4-(trifluoromethoxy)phenyl)-3,4-dihydro-2H-pyrano[3,2-b]pyridin-4-yl)-1H-pyrrolo[2,3-b]pyridine-2-carboxamide;
(S)—N-(4-(4-(trifluoromethoxy)phenyl)-3,4-dihydro-2H-pyrano[3,2-b]pyridin-4-yl)-1H-pyrrolo[2,3-b]pyridine-3-carboxamide;
(S)—N-(4-(4-(trifluoromethoxy)phenyl)-3,4-dihydro-2H-pyrano[3,2-b]pyridin-4-yl)-1H-pyrrolo[2,3-b]pyridine-4-carboxamide;
(S)—N-(4-(4-(trifluoromethoxy)phenyl)-3,4-dihydro-2H-pyrano[3,2-b]pyridin-4-yl)pyrazolo[1,5-a]pyridine-2-carboxamide;
(S)—N-(4-(4-(trifluoromethoxy)phenyl)-3,4-dihydro-2H-pyrano[3,2-b]pyridin-4-yl)-6,7-dihydro-5H-pyrazolo[5,1-b][1,3]oxazine-2-carboxamide;
(S)—N-(4-(4-(trifluoromethoxy)phenyl)-3,4-dihydro-2H-pyrano[3,2-b]pyridin-4-yl)quinoline-8-carboxamide;
(S)-5-methyl-N-(4-(4-(trifluoromethoxy)phenyl)-3,4-dihydro-2H-pyrano[3,2-b]pyridin-4-yl)-1H-indole-2-carboxamide;
(S)-1-oxo-N-(4-(4-(trifluoromethoxy)phenyl)-3,4-dihydro-2H-pyrano[3,2-b]pyridin-4-yl)-1,2-dihydroisoquinoline-6-carboxamide;
(S)-7-cyclopropyl-N-(4-(4-(trifluoromethoxy)phenyl)-3,4-dihydro-2H-pyrano[3,2-b]pyridin-4-yl)pyrazolo[1,5-a]pyrimidine-2-carboxamide;
(S)-7-methoxy-N-(4-(4-(trifluoromethoxy)phenyl)-3,4-dihydro-2H-pyrano[3,2-b]pyridin-4-yl)quinoline-3-carboxamide;
(S)—N-(4-(4-(trifluoromethoxy)phenyl)-3,4-dihydro-2H-pyrano[3,2-b]pyridin-4-yl)oxazole-4-carboxamide;
(S)-1-methyl-N-(4-(4-(trifluoromethoxy)phenyl)-3,4-dihydro-2H-pyrano[3,2-b]pyridin-4-yl)-1H-pyrazole-3-carboxamide;
(S)-1-methyl-N-(4-(4-(trifluoromethoxy)phenyl)-3,4-dihydro-2H-pyrano[3,2-b]pyridin-4-yl)-1H-pyrazole-5-carboxamide;
(S)—N-(4-(4-(trifluoromethoxy)phenyl)-3,4-dihydro-2H-pyrano[3,2-b]pyridin-4-yl)thiophene-2-carboxamide;
(S)—N-(4-(4-(trifluoromethoxy)phenyl)-3,4-dihydro-2H-pyrano[3,2-b]pyridin-4-yl)thiophene-3-carboxamide;
(S)—N-(4-(4-(trifluoromethoxy)phenyl)-3,4-dihydro-2H-pyrano[3,2-b]pyridin-4-yl)thiazole-4-carboxamide;
(S)—N-(4-(4-(trifluoromethoxy)phenyl)-3,4-dihydro-2H-pyrano[3,2-b]pyridin-4-yl)isothiazole-3-carboxamide;
(S)—N-(4-(4-(trifluoromethoxy)phenyl)-3,4-dihydro-2H-pyrano[3,2-b]pyridin-4-yl)thiazole-5-carboxamide;
(S)—N-(4-(4-(trifluoromethoxy)phenyl)-3,4-dihydro-2H-pyrano[3,2-b]pyridin-4-yl)-1H-indole-3-carboxamide;
(S)—N-(4-(4-(trifluoromethoxy)phenyl)-3,4-dihydro-2H-pyrano[3,2-b]pyridin-4-yl)-1H-indole-6-carboxamide;
(S)—N-(4-(4-(trifluoromethoxy)phenyl)-3,4-dihydro-2H-pyrano[3,2-b]pyridin-4-yl)benzofuran-2-carboxamide;
(S)—N-(4-(4-(trifluoromethoxy)phenyl)-3,4-dihydro-2H-pyrano[3,2-b]pyridin-4-yl)-1H-benzo[d]imidazole-5-carboxamide;
(S)—N-(4-(4-(trifluoromethoxy)phenyl)-3,4-dihydro-2H-pyrano[3,2-b]pyridin-4-yl)-1H-benzo[d]imidazole-4-carboxamide;
(S)—N-(4-(4-(trifluoromethoxy)phenyl)-3,4-dihydro-2H-pyrano[3,2-b]pyridin-4-yl)imidazo[1,2-a]pyridine-2-carboxamide;
(S)—N-(4-(4-(trifluoromethoxy)phenyl)-3,4-dihydro-2H-pyrano[3,2-b]pyridin-4-yl)imidazo[1,2-a]pyridine-6-carboxamide;
(S)—N-(4-(4-(trifluoromethoxy)phenyl)-3,4-dihydro-2H-pyrano[3,2-b]pyridin-4-yl)-1H-benzo[d]imidazole-2-carboxamide;
(S)—N-(4-(4-(trifluoromethoxy)phenyl)-3,4-dihydro-2H-pyrano[3,2-b]pyridin-4-yl)-1H-pyrazolo[3,4-b]pyridine-3-carboxamide;
(S)—N-(4-(4-(trifluoromethoxy)phenyl)-3,4-dihydro-2H-pyrano[3,2-b]pyridin-4-yl)imidazo[1,2-b]pyridazine-2-carboxamide;
(S)—N-(4-(4-(trifluoromethoxy)phenyl)-3,4-dihydro-2H-pyrano[3,2-b]pyridin-4-yl)pyrazolo[1,5-a]pyrimidine-3-carboxamide;
N—-((S)-4-(4-(trifluoromethoxy)phenyl)-3,4-dihydro-2H-pyrano[3,2-b]pyridin-4-yl)indoline-2-carboxamide;
N—-((S)-4-(4-(trifluoromethoxy)phenyl)-3,4-dihydro-2H-pyrano[3,2-b]pyridin-4-yl)-2,3-dihydrobenzofuran-2-carboxamide;
(S)—N-(4-(4-(trifluoromethoxy)phenyl)-3,4-dihydro-2H-pyrano[3,2-b]pyridin-4-yl)isoquinoline-1-carboxamide;
(S)—N-(4-(4-(trifluoromethoxy)phenyl)-3,4-dihydro-2H-pyrano[3,2-b]pyridin-4-yl)isoquinoline-3-carboxamide;
(S)—N-(4-(4-(trifluoromethoxy)phenyl)-3,4-dihydro-2H-pyrano[3,2-b]pyridin-4-yl)isoquinoline-5-carboxamide;
(S)—N-(4-(4-(trifluoromethoxy)phenyl)-3,4-dihydro-2H-pyrano[3,2-b]pyridin-4-yl)isoquinoline-6-carboxamide;
(S)—N-(4-(4-(trifluoromethoxy)phenyl)-3,4-dihydro-2H-pyrano[3,2-b]pyridin-4-yl)quinoline-2-carboxamide;
(S)—N-(4-(4-(trifluoromethoxy)phenyl)-3,4-dihydro-2H-pyrano[3,2-b]pyridin-4-yl)quinoline-3-carboxamide;
(S)—N-(4-(4-(trifluoromethoxy)phenyl)-3,4-dihydro-2H-pyrano[3,2-b]pyridin-4-yl)quinoline-6-carboxamide;
(S)—N-(4-(4-(trifluoromethoxy)phenyl)-3,4-dihydro-2H-pyrano[3,2-b]pyridin-4-yl)quinoline-7-carboxamide;
(S)—N-(4-(4-(trifluoromethoxy)phenyl)-3,4-dihydro-2H-pyrano[3,2-b]pyridin-4-yl)quinoxaline-2-carboxamide;
(S)-1-methyl-N-(4-(4-(trifluoromethoxy)phenyl)-3,4-dihydro-2H-pyrano[3,2-b]pyridin-4-yl)-1H-indole-2-carboxamide;
(S)-1-methyl-N-(4-(4-(trifluoromethoxy)phenyl)-3,4-dihydro-2H-pyrano[3,2-b]pyridin-4-yl)-1H-indole-3-carboxamide;
(S)-1-methyl-N-(4-(4-(trifluoromethoxy)phenyl)-3,4-dihydro-2H-pyrano[3,2-b]pyridin-4-yl)-1H-indole-5-carboxamide;
(S)—N-(4-(4-(trifluoromethoxy)phenyl)-3,4-dihydro-2H-pyrano[3,2-b]pyridin-4-yl)-2H-chromene-3-carboxamide;
(S)-1-methyl-N-(4-(4-(trifluoromethoxy)phenyl)-3,4-dihydro-2H-pyrano[3,2-b]pyridin-4-yl)-1H-benzo[d]imidazole-2-carboxamide;
(S)-1-methyl-N-(4-(4-(trifluoromethoxy)phenyl)-3,4-dihydro-2H-pyrano[3,2-b]pyridin-4-yl)-1H-indazole-4-carboxamide;
(S)-1-methyl-N-(4-(4-(trifluoromethoxy)phenyl)-3,4-dihydro-2H-pyrano[3,2-b]pyridin-4-yl)-1H-indazole-5-carboxamide;
(S)-1-methyl-N-(4-(4-(trifluoromethoxy)phenyl)-3,4-dihydro-2H-pyrano[3,2-b]pyridin-4-yl)-1H-indazole-6-carboxamide;
(S)-2-methyl-N-(4-(4-(trifluoromethoxy)phenyl)-3,4-dihydro-2H-pyrano[3,2-b]pyridin-4-yl)-2H-indazole-4-carboxamide;
(S)-2-methyl-N-(4-(4-(trifluoromethoxy)phenyl)-3,4-dihydro-2H-pyrano[3,2-b]pyridin-4-yl)-2H-indazole-6-carboxamide;
(S)-2-methyl-N-(4-(4-(trifluoromethoxy)phenyl)-3,4-dihydro-2H-pyrano[3,2-b]pyridin-4-yl)-2H-indazole-7-carboxamide;
(S)—N-(4-(4-(trifluoromethoxy)phenyl)-3,4-dihydro-2H-pyrano[3,2-b]pyridin-4-yl)benzo[b]thiophene-2-carboxamide;
(S)—N-(4-(4-(trifluoromethoxy)phenyl)-3,4-dihydro-2H-pyrano[3,2-b]pyrid in-4-yl)benzo[b]thiophene-3-carboxamide;
(S)—N-(4-(4-(trifluoromethoxy)phenyl)-3,4-dihydro-2H-pyrano[3,2-b]pyridin-4-yl)benzo[c][1,2,5]thiadiazole-5-carboxamide;
(S)-2-methyl-N-(4-(4-(trifluoromethoxy)phenyl)-3,4-dihydro-2H-pyrano[3,2-b]pyridin-4-yl)-4,5,6,7-tetrahydro-2H-indazole-3-carboxamide;
(S)-1-oxo-N-(4-(4-(trifluoromethoxy)phenyl)-3,4-dihydro-2H-pyrano[3,2-b]pyridin-4-yl)-1,2-dihydroisoquinoline-7-carboxamide;
(S)-1-oxo-N-(4-(4-(trifluoromethoxy)phenyl)-3,4-dihydro-2H-pyrano[3,2-b]pyridin-4-yl)-1,2-dihydroisoquinoline-4-carboxamide;
(S)-4-(oxazol-5-yl)-N-(4-(4-(trifluoromethoxy)phenyl)-3,4-dihydro-2H-pyrano[3,2-b]pyridin-4-yl)benzamide;
(S)-5-methoxy-N-(4-(4-(trifluoromethoxy)phenyl)-3,4-dihydro-2H-pyrano[3,2-b]pyridin-4-yl)-1H-pyrrolo[2,3-c]pyridine-2-carboxamide;
(S)-3-(5-methyl-1,2,4-oxadiazol-3-yl)-N-(4-(4-(trifluoromethoxy)phenyl)-3,4-dihydro-2H-pyrano[3,2-b]pyridin-4-yl)benzamide;
(S)-4-(5-methyl-1,2,4-oxadiazol-3-yl)-N-(4-(4-(trifluoromethoxy)phenyl)-3,4-dihydro-2H-pyrano[3,2-b]pyridin-4-yl)benzamide;
(S)-3-(2-oxopyrrolidin-1-yl)-N-(4-(4-(trifluoromethoxy)phenyl)-3,4-dihydro-2H-pyrano[3,2-b]pyridin-4-yl)benzamide;
(S)-6-methoxy-N-(4-(4-(trifluoromethoxy)phenyl)-3,4-dihydro-2H-pyrano[3,2-b]pyridin-4-yl)-2H-chromene-3-carboxamide;
(S)-ethyl 4-((4-(3-fluoro-4-(trifluoromethoxy)phenyl)-3,4-dihydro-2H-pyrano[3,2-b]pyridin-4-yl)carbamoyl)thiazole-2-carboxylate;
(S)-methyl 3-((4-(3-fluoro-4-(trifluoromethoxy)phenyl)-3,4-dihydro-2H-pyrano[3,2-b]pyridin-4-yl)carbamoyl)benzoate;
(S)—N-(4-(3-fluoro-4-(trifluoromethoxy)phenyl)-3,4-dihydro-2H-pyrano[3,2-b]pyridin-4-yl)-5-(2H-tetrazol-5-yl)picolinamide;
(S)—N-(4-(3-fluoro-4-(trifluoromethoxy)phenyl)-3,4-dihydro-2H-pyrano[3,2-b]pyridin-4-yl)-5-(1,2,4-oxadiazol-3-yl)picolinamide;
(S)—N-(4-(3-fluoro-4-(trifluoromethoxy)phenyl)-3,4-dihydro-2H-pyrano[3,2-b]pyridin-4-yl)-5-(5-oxo-4,5-dihydro-1,2,4-oxadiazol-3-yl)picolinamide;
(S)-5-amino-N-(4-(3-fluoro-4-(trifluoromethoxy)phenyl)-3,4-dihydro-2H-pyrano[3,2-b]pyridin-4-yl)picolinamide;
(S)-methyl 3-(N-(4-(3-fluoro-4-(trifluoromethoxy)phenyl)-3,4-dihydro-2H-pyrano[3,2-b]pyridin-4-yl)sulfamoyl)benzoate;
(S)-methyl 4-(N-(4-(3-fluoro-4-(trifluoromethoxy)phenyl)-3,4-dihydro-2H-pyrano[3,2-b]pyridin-4-yl)sulfamoyl)benzoate;
(S)-4-((4-(3-fluoro-4-(trifluoromethyl)phenyl)-3,4-dihydro-2H-pyrano[3,2-b]pyridin-4-yl)carbamoyl)-2-methylbenzoic acid;
(S)-2-methyl-4-((4-(4-(trifluoromethyl)phenyl)-3,4-dihydro-2H-pyrano[3,2-b]pyridin-4-yl)carbamoyl)benzoic acid;
(S)-4-((4-(3-fluoro-4-(trifluoromethoxy)phenyl)-3,4-dihydro-2H-pyrano[3,2-b]pyridin-4-yl)carbamoyl)-2-vinylbenzoic acid;
(S)-5-((4-(3-fluoro-4-(trifluoromethoxy)phenyl)-3,4-dihydro-2H-pyrano[3,2-b]pyridin-4-yl)carbamoyl)-[1,1′-biphenyl]-2-carboxylic acid;
(S)-2-ethyl-4-((4-(3-fluoro-4-(trifluoromethoxy)phenyl)-3,4-dihydro-2H-pyrano[3,2-b]pyridin-4-yl)carbamoyl)benzoic acid;
(S)-4-((4-(3-fluoro-4-(trifluoromethoxy)phenyl)-3,4-dihydro-2H-pyrano[3,2-b]pyridin-4-yl)carbamoyl)-2-isopropylbenzoic acid;
(S)-4-((4-(3-fluoro-4-(trifluoromethoxy)phenyl)-3,4-dihydro-2H-pyrano[3,2-b]pyridin-4-yl)carbamoyl)-2-isobutylbenzoic acid;
(S)-4-((4-(3-fluoro-4-(trifluoromethoxy)phenyl)-3,4-dihydro-2H-pyrano[3,2-b]pyridin-4-yl)carbamoyl)-2-(methylamino)benzoic acid;
(S)-methyl 2-acetamido-4-((4-(3-fluoro-4-(trifluoromethoxy)phenyl)-3,4-dihydro-2H-pyrano[3,2-b]pyridin-4-yl)carbamoyl)benzoate;
(S)—N4-(4-(3-fluoro-4-(trifluoromethoxy)phenyl)-3,4-dihydro-2H-pyrano[3,2-b]pyridin-4-yl)-N1,2-dimethylterephthalamide;
(S)—N1-(tert-butyl)-N4-(4-(3-fluoro-4-(trifluoromethoxy)phenyl)-3,4-dihydro-2H-pyrano[3,2-b]pyridin-4-yl)-2-methylterephthalamide;
(S)-4-((4-(3-fluoro-4-(trifluoromethoxy)phenyl)-3,4-dihydro-2H-pyrano[3,2-b]pyridin-4-yl)carbamoyl)-2-hydroxybenzoic acid;
(S)-4-((4-(3-fluoro-4-(trifluoromethoxy)phenyl)-3,4-dihydro-2H-pyrano[3,2-b]pyridin-4-yl)carbamoyl)-1-methyl-1H-pyrrole-2-carboxylic acid;
(S)-2-cyclopropyl-4-((4-(3-fluoro-4-(trifluoromethoxy)phenyl)-3,4-dihydro-2H-pyrano[3,2-b]pyridin-4-yl)carbamoyl)benzoic acid;
(S)-5-((4-(3-fluoro-4-(trifluoromethoxy)phenyl)-3,4-dihydro-2H-pyrano[3,2-b]pyridin-4-yl)carbamoyl)-2-methyl-1H-pyrrole-3-carboxylic acid;
(S)-2-(cyclopropylamino)-4-((4-(3-fluoro-4-(trifluoromethoxy)phenyl)-3,4-dihydro-2H-pyrano[3,2-b]pyridin-4-yl)carbamoyl)benzoic acid;
(S)-5-((4-(3-fluoro-4-(trifluoromethoxy)phenyl)-3,4-dihydro-2H-pyrano[3,2-b]pyridin-4-yl)carbamoyl)-1,2-dimethyl-1H-pyrrole-3-carboxylic acid;
(S)-2-fluoro-4-((4-(3-fluoro-4-(trifluoromethoxy)phenyl)-3,4-dihydro-2H-pyrano[3,2-b]pyridin-4-yl)carbamoyl)-6-(methylamino)benzoic acid;
(S)-methyl 5-((4-(3-fluoro-4-(trifluoromethoxy)phenyl)-3,4-dihydro-2H-pyrano[3,2-b]pyridin-4-yl)carbamoyl)-1-methyl-1H-pyrrole-2-carboxylate;
(S)-5-((4-(3-fluoro-4-(trifluoromethoxy)phenyl)-3,4-dihydro-2H-pyrano[3,2-b]pyridin-4-yl)carbamoyl)-1H-pyrrole-2-carboxylic acid;
(S)—N2-(4-(3-fluoro-4-(trifluoromethoxy)phenyl)-3,4-dihydro-2H-pyrano[3,2-b]pyridin-4-yl)-N5-(phenylsulfonyl)pyridine-2,5-dicarboxamide;
(S)—N-(4-(3-fluoro-4-(trifluoromethoxy)phenyl)-3,4-dihydro-2H-pyrano[3,2-b]pyridin-4-yl)furan-2-carboxamide;
(S)—N1-(4-(3-fluoro-4-(trifluoromethoxy)phenyl)-3,4-dihydro-2H-pyrano[3,2-b]pyridin-4-yl)-N3-methylisophthalamide;
(S)-6-((4-(3-fluoro-4-(trifluoromethoxy)phenyl)-3,4-dihydro-2H-pyrano[3,2-b]pyridin-4-yl)carbamoyl)-2-methylnicotinic acid;
(S)-4-((4-(3-fluoro-4-(trifluoromethoxy)phenyl)-3,4-dihydro-2H-pyrano[3,2-b]pyridin-4-yl)carbamoyl)-2,6-dimethylbenzoic acid;
(S)-4-((4-(3-fluoro-4-(trifluoromethoxy)phenyl)-3,4-dihydro-2H-pyrano[3,2-b]pyridin-4-yl)carbamoyl)-3-methoxybenzoic acid;
(S)-5-((4-(3-fluoro-4-(trifluoromethoxy)phenyl)-3,4-dihydro-2H-pyrano[3,2-b]pyridin-4-yl)carbamoyl)-2-methoxybenzoic acid;
(S)-3-((4-(3-fluoro-4-(trifluoromethoxy)phenyl)-3,4-dihydro-2H-pyrano[3,2-b]pyridin-4-yl)carbamoyl)-4-methoxybenzoic acid;
(S)-4-(N-(4-(3-fluoro-4-(trifluoromethoxy)phenyl)-3,4-dihydro-2H-pyrano[3,2-b]pyridin-4-yl)sulfamoyl)benzoic acid;
(S)-2-methyl-4-((4-(4-(trifluoromethoxy)phenyl)-3,4-dihydro-2H-pyrano[3,2-b]pyridin-4-yl)carbamoyl)benzoic acid;
(S)-4-((4-(3-fluoro-4-(trifluoromethoxy)phenyl)-3,4-dihydro-2H-pyrano[3,2-b]pyridin-4-yl)carbamoyl)-1H-pyrrole-2-carboxylic acid;
(S)-2-amino-4-((4-(3-fluoro-4-(trifluoromethoxy)phenyl)-3,4-dihydro-2H-pyrano[3,2-b]pyridin-4-yl)carbamoyl)benzoic acid;
(S)-6-((4-(3-fluoro-4-(trifluoromethoxy)phenyl)-3,4-dihydro-2H-pyrano[3,2-b]pyridin-4-yl)carbamoyl)-1-methyl-1H-indole-2-carboxylic acid;
(S)-2-acetamido-4-((4-(3-fluoro-4-(trifluoromethoxy)phenyl)-3,4-dihydro-2H-pyrano[3,2-b]pyridin-4-yl)carbamoyl)benzoic acid;
(S)-5-((4-(3-fluoro-4-(trifluoromethoxy)phenyl)-3,4-dihydro-2H-pyrano[3,2-b]pyridin-4-yl)carbamoyl)-1-methyl-1H-pyrrole-2-carboxylic acid;
(S)-6-((4-(3-fluoro-4-(trifluoromethoxy)phenyl)-3,4-dihydro-2H-pyrano[3,2-b]pyridin-4-yl)carbamoyl)-1H-indole-2-carboxylic acid;
(S)-5-((4-(3-fluoro-4-(trifluoromethoxy)phenyl)-3,4-dihydro-2H-pyrano[3,2-b]pyridin-4-yl)carbamoyl)picolinic acid;
(S)-4-((4-(3-fluoro-4-(trifluoromethoxy)phenyl)-3,4-dihydro-2H-pyrano[3,2-b]pyridin-4-yl)carbamoyl)thiazole-2-carboxylic acid;
(S)-2-((4-(3-fluoro-4-(trifluoromethoxy)phenyl)-3,4-dihydro-2H-pyrano[3,2-b]pyridin-4-yl)carbamoyl)isonicotinic acid;
(S)-2-fluoro-4-((4-(3-fluoro-4-(trifluoromethoxy)phenyl)-3,4-dihydro-2H-pyrano[3,2-b]pyridin-4-yl)carbamoyl)benzoic acid;
(S)-4-((4-(3-fluoro-4-(trifluoromethoxy)phenyl)-3,4-dihydro-2H-pyrano[3,2-b]pyridin-4-yl)carbamoyl)-2-methylbenzoic acid;
(S)-6-((4-(3-fluoro-4-(trifluoromethoxy)phenyl)-3,4-dihydro-2H-pyrano[3,2-b]pyridin-4-yl)carbamoyl)-4-methylnicotinic acid;
(S)-5-((4-(3-fluoro-4-(trifluoromethoxy)phenyl)-3,4-dihydro-2H-pyrano[3,2-b]pyridin-4-yl)carbamoyl)-1H-pyrrole-3-carboxylic acid;
(S)-5-((4-(3-fluoro-4-(trifluoromethoxy)phenyl)-3,4-dihydro-2H-pyrano[3,2-b]pyridin-4-yl)carbamoyl)-1-methyl-1H-pyrrole-3-carboxylic acid;
(S)-5-((4-(3-fluoro-4-(trifluoromethoxy)phenyl)-3,4-dihydro-2H-pyrano[3,2-b]pyridin-4-yl)carbamoyl)thiophene-2-carboxylic acid; or
(S)-4-((4-(3-fluoro-4-(trifluoromethoxy)phenyl)-3,4-dihydro-2H-pyrano[3,2-b]pyridin-4-yl)carbamoyl)-2-methoxybenzoic acid; or
the pharmaceutically-acceptable salt thereof, the tautomer thereof, the pharmaceutically-acceptable salt of the tautomer, the stereoisomer thereof, or the mixture thereof.

47. The compound of claim 1, wherein the compound is
(S)-1-(4-(3-fluoro-4-(trifluoromethoxy)phenyl)-3,4-dihydro-2H-pyrano[3,2-b]pyridin-4-yl)-3-(pyrimidin-2-yl)urea;
(S)-1-(4-(3-fluoro-4-(trifluoromethoxy)phenyl)-3,4-dihydro-2H-pyrano[3,2-b]pyridin-4-yl)-3-(pyrimidin-5-yl)urea;
(S)—N-(4-(4′-cyano-[1,1′-biphenyl]-4-yl)-3,4-dihydro-2H-pyrano[3,2-b]pyridin-4-yl)-2-oxo-2,3-dihydrobenzo[d]oxazole-5-carboxamide;
(S)—N-(4-(3-fluoro-4-(trifluoromethoxy)phenyl)-3,4-dihydro-2H-pyrano[3,2-b]pyridin-4-yl)-4-(hydroxymethyl)benzamide;
(S)-4-((4-(3-fluoro-4-(trifluoromethoxy)phenyl)-3,4-dihydro-2H-pyrano[3,2-b]pyridin-4-yl)carbamoyl)benzoic acid;
(S)—N-(4-(3-fluoro-4-(trifluoromethoxy)phenyl)-3,4-dihydro-2H-pyrano[3,2-b]pyridin-4-yl)-5-(5-oxo-4,5-dihydro-1,2,4-oxadiazol-3-yl)picolinamide;
(S)-4-((4-(3-fluoro-4-(trifluoromethoxy)phenyl)-3,4-dihydro-2H-pyrano[3,2-b]pyridin-4-yl)carbamoyl)-2-vinylbenzoic acid;
(S)-2-ethyl-4-((4-(3-fluoro-4-(trifluoromethoxy)phenyl)-3,4-dihydro-2H-pyrano[3,2-b]pyridin-4-yl)carbamoyl)benzoic acid;
(S)-6-((4-(3-fluoro-4-(trifluoromethoxy)phenyl)-3,4-dihydro-2H-pyrano[3,2-b]pyridin-4-yl)carbamoyl)-2-methylnicotinic acid;
(S)-4-((4-(3-fluoro-4-(trifluoromethoxy)phenyl)-3,4-dihydro-2H-pyrano[3,2-b]pyridin-4-yl)carbamoyl)-3-methoxybenzoic acid;
(S)-2-methyl-4-((4-(4-(trifluoromethoxy)phenyl)-3,4-dihydro-2H-pyrano[3,2-b]pyridin-4-yl)carbamoyl)benzoic acid;
(S)-2-amino-4-((4-(3-fluoro-4-(trifluoromethoxy)phenyl)-3,4-dihydro-2H-pyrano[3,2-b]pyridin-4-yl)carbamoyl)benzoic acid;
(S)-6-((4-(3-fluoro-4-(trifluoromethoxy)phenyl)-3,4-dihydro-2H-pyrano[3,2-b]pyridin-4-yl)carbamoyl)-1-methyl-1H-indole-2-carboxylic acid; or
(S)-4-((4-(3-fluoro-4-(trifluoromethoxy)phenyl)-3,4-dihydro-2H-pyrano[3,2-b]pyridin-4-yl)carbamoyl)-2-methylbenzoic acid; or
the pharmaceutically-acceptable salt thereof, the tautomer thereof, the pharmaceutically-acceptable salt of the tautomer, the stereoisomer thereof, or the mixture thereof.

48. The compound of claim 1, wherein the compound is (S)—N-(4-(4′-cyano-[1,1′-biphenyl]-4-yl)-3,4-dihydro-2H-pyrano[3,2-b]pyridin-4-yl)-2-oxo-2,3-dihydrobenzo[d]oxazole-5-carboxamide, or
the pharmaceutically-acceptable salt thereof, the tautomer thereof, the pharmaceutically-acceptable salt of the tautomer, the stereoisomer thereof, or the mixture thereof.

49. The compound of claim 1, wherein the compound is (S)-1-(4-(3-fluoro-4-(trifluoromethoxy)phenyl)-3,4-dihydro-2H-pyrano[3,2-b]pyridin-4-yl)-3-(pyrimidin-2-yl)urea, or the pharmaceutically-acceptable salt thereof, the tautomer thereof, the pharmaceutically-acceptable salt of the tautomer, the stereoisomer thereof, or the mixture thereof.

50. The compound of claim 1, wherein the compound is (S)—N-(4-(3-fluoro-4-(trifluoromethoxy)phenyl)-3,4-dihydro-2H-pyrano[3,2-b]pyridin-4-yl)-5-(5-oxo-4,5-dihydro-1,2,4-oxadiazol-3-yl)picolinamide, or the pharmaceutically-acceptable salt thereof, the tautomer thereof, the pharmaceutically-acceptable salt of the tautomer, the stereoisomer thereof, or the mixture thereof.

51. The compound of claim 1, wherein the compound is (S)-2-ethyl-4-((4-(3-fluoro-4-(trifluoromethoxy)phenyl)-3,4-dihydro-2H-pyrano[3,2-b]pyridin-4-yl)carbamoyl)benzoic acid, or the pharmaceutically-acceptable salt thereof, the tautomer thereof, the pharmaceutically-acceptable salt of the tautomer, the stereoisomer thereof, or the mixture thereof.

52. The compound of claim 1, wherein the compound is (S)-4-((4-(3-fluoro-4-(trifluoromethoxy)phenyl)-3,4-dihydro-2H-pyrano[3,2-b]pyridin-4-yl)carbamoyl)-3-methoxybenzoic acid, or the pharmaceutically-acceptable salt thereof, the tautomer thereof, the pharmaceutically-acceptable salt of the tautomer, the stereoisomer thereof, or the mixture thereof.

53. The compound of claim 1, wherein the compound is (S)-6-((4-(3-fluoro-4-(trifluoromethoxy)phenyl)-3,4-dihydro-2H-pyrano[3,2-b]pyridin-4-yl)carbamoyl)-1-methyl-1H-indole-2-carboxylic acid, or the pharmaceutically-acceptable salt thereof, the tautomer thereof, the pharmaceutically-acceptable salt of the tautomer, the stereoisomer thereof, or the mixture thereof.

54. The compound of claim 1, wherein the compound is (S)-2-amino-4-((4-(3-fluoro-4-(trifluoromethoxy)phenyl)-3,4-dihydro-2H-pyrano[3,2-b]pyridin-4-yl)carbamoyl)benzoic acid, or the pharmaceutically-acceptable salt thereof, the tautomer thereof, the pharmaceutically-acceptable salt of the tautomer, the stereoisomer thereof, or the mixture thereof.

55. The compound of claim 1, wherein the compound is (S)-4-((4-(3-fluoro-4-(trifluoromethoxy)phenyl)-3,4-dihydro-2H-pyrano[3,2-b]pyridin-4-yl)carbamoyl)-2-methylbenzoic acid, or the pharmaceutically-acceptable salt thereof, the tautomer thereof, the pharmaceutically-acceptable salt of the tautomer, the stereoisomer thereof, or the mixture thereof.

56. The compound or tautomer of claim 1 in a neutral form.

57. The pharmaceutically-acceptably salt of the compound or the pharmaceutically acceptable salt of the tautomer of claim 1.

58. A pharmaceutical composition comprising the compound according to claim 1 or the pharmaceutically-acceptable salt thereof, the tautomer thereof, the pharmaceutically-acceptable salt of the tautomer, the stereoisomer thereof, or the mixture thereof, and a pharmaceutically-acceptable diluent or carrier.

59. A method of treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders in a subject, the method comprising administering the compound according to claim 1 or the pharmaceutically-acceptable salt thereof, the tautomer thereof, the pharmaceutically-acceptable salt of the tautomer, the stereoisomer thereof, or the mixture thereof to the subject.

60. The method of claim 59, wherein the subject is suffering from migraine pain.
